SEQUENCE LISTING (5 sequences)

SEQ ID NO: 1 - protein, 238 aa, Homo sapiens
  REGION 1..238: note = MISC_FEATURE - Light chain of antibody hu2H11_R35R74
MGWSCIILFLVATATGVHSDVVMTQTPLSLSVTLGQPASISCKSSQSLIHSDGRTYLNWLLQRPGQSPKRLIYLVSRLDSGVPDRFTGSGAGTDFTLKISRVEAEDLGVYYCWQGSHFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 467 aa, Homo sapiens
  REGION 1..467: note = MISC_FEATURE - Heavy chain of antibody hu2H11_R35R74
MGWSCIILFLVATATGVHSQVQLVQSGAEVVKPGASVKISCKASGYTFTAYYMHWVKQSPVQSLEWIGLVNPYNGFSSYNQKFQGKASLTVDRSSSTAYMELHSLTSEDSAVYYCAREFYGYRYFDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - skipped

SEQ ID NO: 4 - protein, 4 aa, synthetic construct
  REGION 1..4: note = non-substituted amino acid
GFLG

SEQ ID NO: 5 - protein, 4 aa, synthetic construct
  REGION 1..4: note = non-substituted amino acid
ALAL

(19) United States
(12) Patent Application Publication
(10) Pub. No.: US 2025/0205348 A1
(43) Pub. Date: Jun. 26, 2025

(54) NOVEL PEPTIDIC LINKERS AND CRYPTOPHYCIN CONJUGATES, THEIR PREPARATION AND THEIR THERAPEUTIC USE

(71) Applicant: SANOFI, Paris (FR)

(72) Inventors: Hervé BOUCHARD, Chilly-Mazarin (FR); Marie-Priscille BRUN, Chilly-Mazarin (FR); Philippe HUBERT, Chilly-Mazarin (FR)

(21) Appl. No.: 18/631,600

(22) Filed: Apr. 10, 2024

Related U.S. Application Data
(62) Division of application No. 17/210,072, filed on Mar. 23, 2021, now Pat. No. 11,998,611.
Application No. 17/210,072 is a division of application No. 16/673,367, filed on Nov. 4, 2019, now Pat. No. 11,007,275.
Application No. 16/673,367 is a division of application No. 15/975,423, filed on May 9, 2018, now abandoned.

(30) Foreign Application Priority Data
May 10, 2017 (EP) 17305531.0

(51) Int. Cl. A61K 47/65; A61K 38/15; A61K 47/10; A61K 47/22; A61K 47/64; A61K 47/68; A61P 35/00; C07D 405/06; C07D 405/14; C07K 11/00

(52) U.S. Cl. CPC A61K 47/65; A61K 38/15; A61K 47/10; A61K 47/22; A61K 47/64; A61K 47/6829; A61K 47/6889; A61P 35/00; C07D 405/06; C07D 405/14; C07K 11/00

(57) ABSTRACT

The present disclosure relates to compounds of formula (1):

RCG1-L-P (i)

wherein RCG1 represents a reactive chemical group being reactive towards a chemical group present on a polypeptide such as an antibody; P represents H, OH or an activated O; and L represents a specific linker. The disclosure also relates to cryptophycin payloads, as well as to cryptophycin conjugates, to compositions containing them and to their therapeutic use, especially as anticancer agents. The disclosure also relates to the process for preparing these conjugates.

### CROSS REFERENCE TO RELATED APPLICATIONS

This application is a divisional of U.S. patent application Ser. No. 17/210,072, filed Mar. 23, 2021, which is a divisional of U.S. patent application Ser. No. 16/673,367, filed Nov. 4, 2019, now U.S. Pat. No. 11,007,275, which is a divisional of U.S. patent application Ser. No. 15/975,423, filed May 9, 2018, which claims priority to European Patent Application No. 17305531.0, filed May 10, 2017, the entirety of which is hereby incorporated herein by reference.

### SEQUENCE LISTING

The instant application contains a Sequence Listing which has been submitted electronically in XML format and is hereby incorporated by reference in its entirety. Said XML file, created on Apr. 9, 2024, is named 753824_SA9-239DIV3_ST26.xml and is 6,972 bytes in size.

These linkers are enzymatically cleaved in the lysosome of cells, by enzymes such as Cathepsin B for example. Cathepsin B is a cysteine proteinase belonging to the papain family and one of the main lysosomal proteinases among mammals. It is involved in protein turnover and in the maintenance of cellular metabolism as well as in several other physiological or pathological processes, like for example tumoral progression. Its over-expression, both at the genetic and proteic levels, has been demonstrated in tumors, the increase of protein leading also to an increase of enzymatic activity. This was the basis of several prodrug approaches of chemotherapeutic agents (Int J Oncology 2013, 42, 373-383) but also of the conception of cleavable linkers for antibody-drug conjugates (ADC) (Bioconj Chem 2002, 13, 855-869). For ADC, one commonly used peptidic linker consists of the sequence Valine-Citrulline (ValCit) joined to the p-aminobenzylic alcohol self-immolative moeity (J Org Chem 2002, 67, 1866-1872), as examplified in WO2011/001052 with example 23. Associated to a cytotoxic drug, the subsequent construct is quite hydrophobic which can be challenging for achieving certain drug-to-antibody ratio (DAR), monomeric purity and ADC stability. Actually, despite extensive conjugation optimization, example 23 of WO2011/00152couldn't be successfully conjugated to provide an ADC with satisfactory DAR and monomeric purity, namely DAR>2 and >95% of monomers.

Since drugs used for ADC—like tubulin or DNA binders—are essentially hydrophobic, there was a need for more hydrophilic peptidic linkers to improve the solubility of the payload—a payload is a compound comprising the cytotoxic drug conjugated to a linker—and thus potentially its reactivity towards antibody conjugation and the stability of the subsequent ADC. Increasing payload solubility should allow to increase the DAR, the monomeric purity and ADC stability, especially in terms of aggregation propensity.

### SUMMARY OF THE DISCLOSURE

The disclosure relates to new peptidic compounds, chosen from compounds of formula (I):

RCG1-L-P (i)

wherein
RCG1 represents a reactive chemical group that is reactive towards a chemical group present on a polypeptide such as an antibody;
P represents a hydrogen atom, —OH or an activated O wherein an activated O is defined below.
L represents a linker of formula (II):

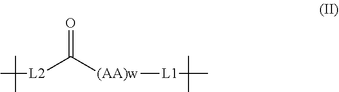

wherein:
L1 is of formula (III):

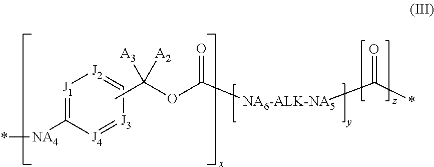

wherein:
   when P represents a hydrogen atom, then x=0 or 1 and y=1 and z=0;
   when P represents —OH, then x=y=z=0;
   when P represents an activated O, then x=1 and y=z=0, or x=y=z=1;
   J₁, J₂, J₃ and J₄ are chosen, independently of each other, from CA₁ and N;
   ALK represents a (C₁-C₁₂)alkylene group, for instance (C₁-C₆)alkylene, such as of the form —(CH₂)ₙ—, n being an integer ranging from 1 to 12 and for example ranging from 1 to 6;
   A₁, A₂, A₃, A₄, A₅, and A₆ represent, independently of each other, a hydrogen atom or a (C₁-C₆)alkyl group, such as a hydrogen atom or a methyl group.
(AA)w represents a sequence of w substituted AAₛ or non-substituted amino acids AAₙₛ connected together via peptide bonds wherein substituted AAₛ or non-substituted AAₙₛ is defined below;
w represents an integer ranging from 1 to 12, for instance from 1 to 6, such as 2 or 3;
if (AA)w contains at least one substituted amino acid AAₛ, then L2 represents a single bond, a (C₁-C₆)alkyl group, a (C₁-C₆)alkyl-(OCH₂CH₂)ᵢ group, a (C₁-C₆)alkyl-(OCH₂CH₂)ᵢ—O(C₁-C₆)alkyl group, a (CH₂CH₂O)ᵢ(C₁-C₆)alkyl group, a CH(SO₃H)—(C₁-C₆)alkyl group, a (C₁-C₆)alkyl-CH(SO₃H) group, a (C₁-C₆)alkyl-cyclohexyl group, a C(═O)—(C₁-C₆)alkyl group, a C(═O)—(C₁-C₆)alkyl-(OCH₂CH₂)ᵢ group, a C(═O)—(C₁-C₆)alkyl-(OCH₂CH₂)ᵢ—O(C₁-C₆)alkyl group, a C(═O)—(CH₂CH₂O)ᵢ(C₁-C₆)alkyl group, a C(═O)—CH(SO₃H)—(C₁-C₆)alkyl group, a C(═O)—(C₁-C₆)alkyl-CH(SO₃H) group, a C(═O)—(C₁-C₆)alkyl-cyclohexyl group, a NA₈-(C₁-C₆)alkyl group, a NA₈-(C₁-C₆)alkyl-(OCH₂CH₂)ᵢ group, a NA₈-(C₁-C₆)alkyl-(OCH₂CH₂)ᵢ—O(C₁-C₆)alkyl group, a NA₈-(CH₂CH₂O)ᵢ(C₁-C₆)alkyl group, a NA₈-(C₁-C₆)alkyl-CH(SO₃H) group, a C(═O)—NA₈-(C₁-C₆)alkyl group, a C(═O)—NA₈-(C₁-C₆)alkyl-(OCH₂CH₂)ᵢ group, a C(═O)—NA₈-(C₁-C₆)alkyl-(OCH₂CH₂)ᵢ—O(C₁-C₆)alkyl group, a C(═O)—NA₈-(CH₂CH₂O)ᵢ(C₁-C₆)alkyl group, a C(═O)—NA₈-(C₁-C₆)alkyl-CH(SO₃H) group, a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ-NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ-NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ-NA₇-(C₁-C₆)alkyl group;
if (AA)w represents a sequence of w non-substituted amino acids AAₙₛ, L2 represents a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ-NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ-NA₇-(C₁-C₆)alkyl group, a NA₈—(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ-NA₇-(C₁-C₆)alkyl group;
A₇ representing a straight or branched, saturated or unsaturated, optionally substituted C₁-C₁₆₀ hydrocarbon chain wherein optionally at least one methylene unit is independently replaced by —NHC(═O)—, —N(alkyl)C(═O)—, —C(═O)NH—, —C(═O)N(alkyl)-, —OC(═O)—, —C(═O)O—, —OC(═O)O—, —CH(OH)—, —CH(SO₃H)—, —O—, —C(═O)—, —S(═O)—, —S(═O)₂—, —NHS(═O)₂—, —N(alkyl)S(═O)₂—, —S(═O)₂NH—, —SO₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂;
   being understood that each A₇ comprising a SO₃H function can be under salt forms such as alkali metal salts, for instance sodium salts (SO₃^{- +}Na);
A₈ representing a hydrogen atom or a (C₁-C₆)alkyl group, for instance a hydrogen atom or a methyl group;
i representing an integer ranging from 1 to 50, for instance ranging from 1 to 35. The disclosure further relates to cryptophycin payloads of formula (IV):

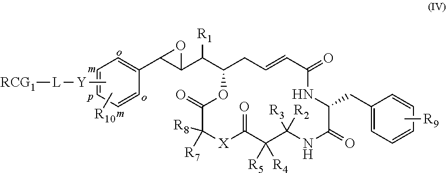

wherein:
R₁ represents a (C₁-C₆)alkyl group;
R₂ and R₃ represent, independently of each other, a hydrogen atom or a (C₁-C₆)alkyl group;
   or alternatively R₂ and R₃ form together with the carbon atom to which they are attached a (C₃-C₆)cycloalkyl or a (C₃-C₆)heterocycloalkyl group;
R₄ and R₅ represent, independently of each other, a hydrogen atom or a (C₁-C₆)alkyl group or a (C₁-C₆)alkyl-NH(R₁₂) group or a (C₁-C₆)alkyl-OH group or a (C₁-C₆)alkyl-SH group or a (C₁-C₆)alkyl-CO₂H group;
   or alternatively R₄ and R₅ form together with the carbon atom to which they are attached a (C₃-C₆)cycloalkyl or a (C₃-C₆)heterocycloalkyl group;
X represents O or N(R₆);
R₆ represents a hydrogen atom or a (C₁-C₆)alkyl group;
R₇ and R₈ represent, independently of each other, a hydrogen atom or a (C₁-C₆)alkyl group or a (C₁-C₆)alkyl-CO₂H group or a (C₁-C₆)alkyl-N(C₁-C₆)alkyl₂ group;
   or alternatively R₇ and R₈ form together with the carbon atom to which they are attached a (C₃-C₆)cycloalkyl group or a (C₃-C₆)heterocycloalkyl group;
R₉ represents at least one substituent of the phenyl nucleus chosen, independently of each other, from: a hydrogen atom, —OH, (C₁-C₄)alkoxy, a halogen atom, —NH₂, —NH(C₁-C₆)alkyl, —N(C₁-C₆)alkyl₂, —NH(C₁-C₆)cycloalkyl or (C₃-C₆)heterocycloalkyl;
R₁₀ represents at least one substituent of the phenyl nucleus chosen from a hydrogen atom and a (C₁-C₄)alkyl group;
Y represents
   —NR₁₁—(C₁-C₆)alkyl-, such as —NR₁₁—(CH₂)ₙ— like

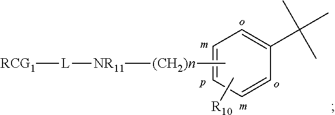

—O—(C₁-C₆)alkyl-, such as —O—(CH₂)ₙ— like

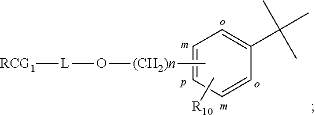

—S—(C₁-C₆)alkyl-, such as —S—(CH₂)ₙ— like

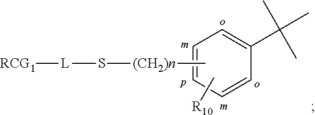

Y being positioned in an ortho (o), meta (m) or para (p) position of the phenyl nucleus;
R₁₁ and R₁₂ represent, independently of each other, a hydrogen atom or (C₁-C₆)alkyl, such as a hydrogen atom or a methyl group;
n represents an integer ranging from 1 to 6.
L is defined as in formula (I) and represents a linker of formula (II);
RCG1 represents a reactive chemical group present at the end of the linker, RCG1 being reactive towards a chemical group present on a polypeptide such as an antibody.

The present disclosure further relates to conjugates of formula (V):

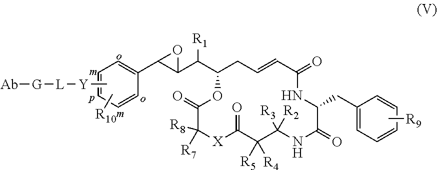

wherein:
R₁, R₂, R₃, R₄, R₅, R₆, R₇, R₈, R₉, R₁₀, R₁₁ and R₁₂ are as defined in formula (IV);
X, Y and L are as defined as in formula (IV);
G represents the product of reaction between RCG1, a reactive chemical group present at the end of the linker and RCG2, an orthogonal reactive chemical group present on the antibody (Ab);
Ab represents an antibody.

Each substituent R₁ to R₁₂ may also adopt one of the spatial configurations (e.g. R or S or alternatively Z or E) as described in the examples.

The compounds of formula (I), (II), (Ill), (IV) and (V) may contain at least one asymetric carbon atoms. They may therefore exist in the form of enantiomers or diastereoisomers. These enantiomers and diastereoisomers, and also mixtures thereof, including racemic mixtures, form part of this disclosure.

The compounds of formula (I), (II), (IV), including those that are illustrated, may exist in the form of bases or of acid-addition salts, for instance of pharmaceutically acceptable acids. For example, if these compounds comprise a SO₃H function, they may exist in the form of SO₃ alkali metal salts, such as of SO₃ sodium salts (SO₃^{- +}Na).

### Definitions

In the context of the present disclosure, certain terms have the following definitions:
alkenyl group: a hydrocarbon group obtained by removing one hydrogen atom from an alkene. The alkenyl group may be linear or branched. Examples that may be mentioned include ethenyl (—CH═CH₂, also named vinyl) and propenyl (—CH₂—CH═CH₂, also named allyl).
alkoxy group: a group —O-alkyl, in which the alkyl group is as defined below;
alkyl group: a linear or branched saturated aliphatic hydrocarbon-based group obtained by removing a hydrogen atom from an alkane. Examples that may be mentioned include methyl, ethyl, propyl, isopropyl, butyl, isobutyl, tert-butyl, pentyl, neopentyl, 2,2-dimethylpropyl and hexyl groups;
alkylene group: a saturated divalent group of empirical formula —CₙH₂ₙ—, obtained by removing two hydrogen atoms from an alkane. The alkylene group may be linear or branched. Examples that may be mentioned include methylene (—CH₂—), ethylene (—CH₂CH₂—), propylene (—CH₂CH₂CH₂—), butylene (—CH₂CH₂CH₂CH₂—) and hexylene (—CH₂CH₂CH₂CH₂CH₂CH₂—) groups or the following branched groups

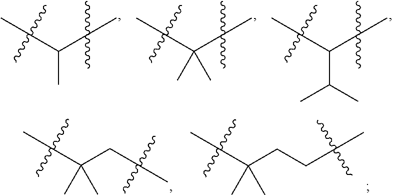

for instance, the alkylene group is of the formula —(CH₂)ₙ—, n representing an integer; in the ranges of values, the limits are included (e.g. a range of the type “n ranging from 1 to 6” or “ranging from 1 to 6” includes limits 1 and 6);
antibody: an antibody with affinity for a biological target, such as a monoclonal antibody. The function of the antibody is to direct the biologically active compound as a cytotoxic compound towards the biological target. The antibody may be monoclonal, polyclonal or multispecific; it may also be an antibody fragment; it may also be a murine, chimeric, humanized or human antibody. An “antibody” may be a natural or conventional antibody in which two heavy chains are linked to each other by disulfide bonds and each heavy chain is linked to a light chain by a disulfide bond (also referred to as a “full-length antibody”). The terms “conventional (or full-length) antibody” refers both to an antibody comprising the signal peptide (or pro-peptide, if any), and to the mature form obtained upon secretion and proteolytic processing of the chain(s). There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

As used herein, the term “antibody” denotes both conventional (full-length) antibodies and fragments thereof, as well as single domain antibodies and fragments thereof, such as variable heavy chain of single domain antibodies. Fragments of (conventional) antibodies typically comprise a portion of an intact antibody, such as the antigen binding region or variable region of the intact antibody, and retain the biological function of the conventional antibody. Examples of such fragments include Fv, Fab, F(ab′)2, Fab′, dsFv, (dsFv)2, scFv, sc(Fv)2, nanobodies and diabodies.

The function of the antibody is to direct the biologically active compound as a cytotoxic compound towards the biological target.
aryl group: a cyclic aromatic group containing ranging from 5 to 10 carbon atoms.

Examples of aryl groups include phenyl, tolyl, xylyl, naphthyl;
biological target: an antigen (or group of antigens) for instance located at the surface of cancer cells or stromal cells associated with this tumour; these antigens may be, for example, a growth factor receptor, an oncogene product or a mutated “tumor suppressant” gene product, an angiogenesis-related molecule or an adhesion molecule;
conjugate: an antibody-drug conjugate or ADC, i.e. a polypeptide such as an antibody to which is covalently attached via a linker at least one molecule of a cytotoxic compound;
cycloalkyl group: a cyclic alkyl group comprising carbon atoms ranging from 3 and 6 engaged in the cyclic structure. Examples that may be mentioned include cyclopropyl, cyclobutyl, cyclopentyl and cyclohexyl groups;
DAR (drug-to-antibody ratio): an average number of cytotoxic molecules attached via a linker to an antibody;
halogen: any of the four elements fluorine, chlorine, bromine and iodine;
heteroaryl group: an aryl group containing carbon atoms ranging from 2 to 10 and heteroatoms ranging from 1 to 5 such as nitrogen, oxygen or sulphur engaged in the ring and connected to the carbon atoms forming the ring. Examples of heteroaryl groups include pyridyl, pyrimidyl, thienyl, imidazolyl, triazolyl, indolyl, imidazo-pyridyl, pyrazolyl;
heterocycloalkyl group: a cycloalkyl group containing carbon atoms ranging from 2 to 8 and heteroatoms ranging from 1 to 3, such as nitrogen, oxygen or sulphur engaged in the ring and connected to the carbon atoms forming the ring. Examples include aziridinyl, pyrrolidinyl, piperidinyl, piperazinyl, morpholinyl, thiomorpholinyl, tetrahydrofuranyl, tetrahydrothiofuranyl, tetrahydropyranyl, azetidinyl, oxetanyl and pyranyl;
linker: a group of atoms or a single bond that can covalently attach a cytotoxic compound to a polypeptide such as an antibody in order to form a conjugate;
payload: a cytotoxic compound to which is covalently attached a linker;
reactive chemical group: a group of atoms that can promote or undergo a chemical reaction;

Further, as stated throughout the present disclosure, a SO₃H function can be under salt forms such as alkali metal salts, for instance sodium salts (SO₃^{- +}Na).

### Abbreviations

ADC: antibody-drug conjugate; ALK: (C₁-C₁₂)alkylene group, for instance (C₁-C₆)alkylene, such as of the form —(CH₂)ₙ—, n being an integer ranging from 1 to 12 and for example ranging from 1 to 6; aq.: aqueous; Ar: argon; AUC: area under the curve; BCN: (1α,8α,9β)-bicyclo[6.1.0]non-4-yne-9-methanol; CHCl₃: chloroform; CH₃CN: acetonitrile; C₀₂: carbon dioxide; CR: complete response; crypto denotes the unit of formula

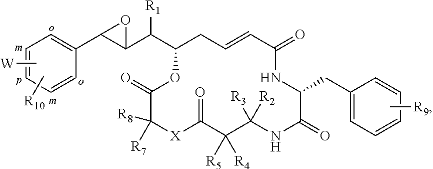

crypto for instance denoting a cryptophycin compound of an example, one of the D₁-D₈ cryptophycin compounds described in WO2011/001052 or one of the D₁-D₁₉ cryptophycin compounds below as described in PCT/EP2016/076603:

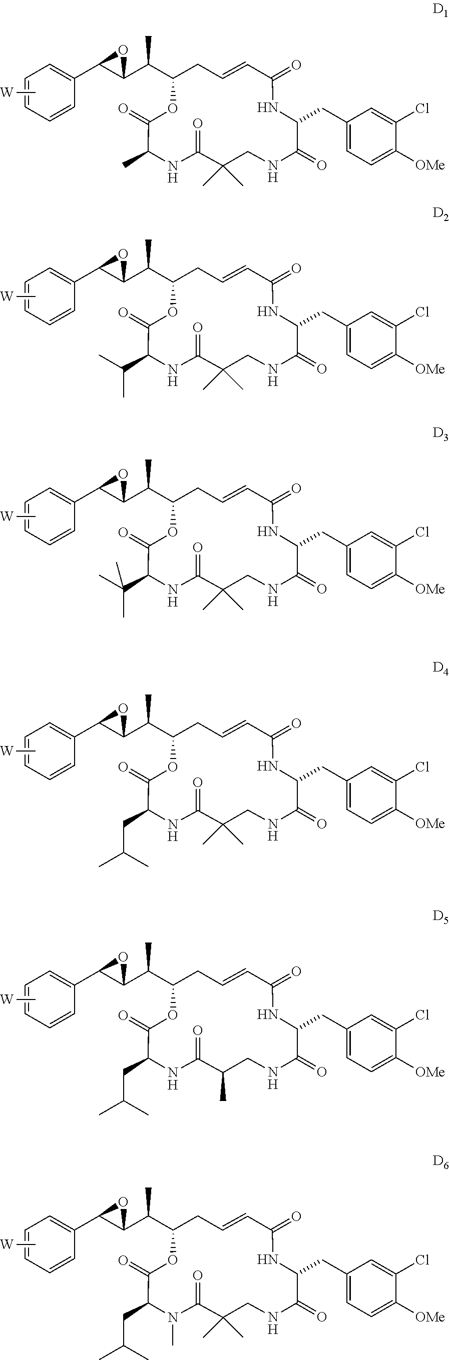

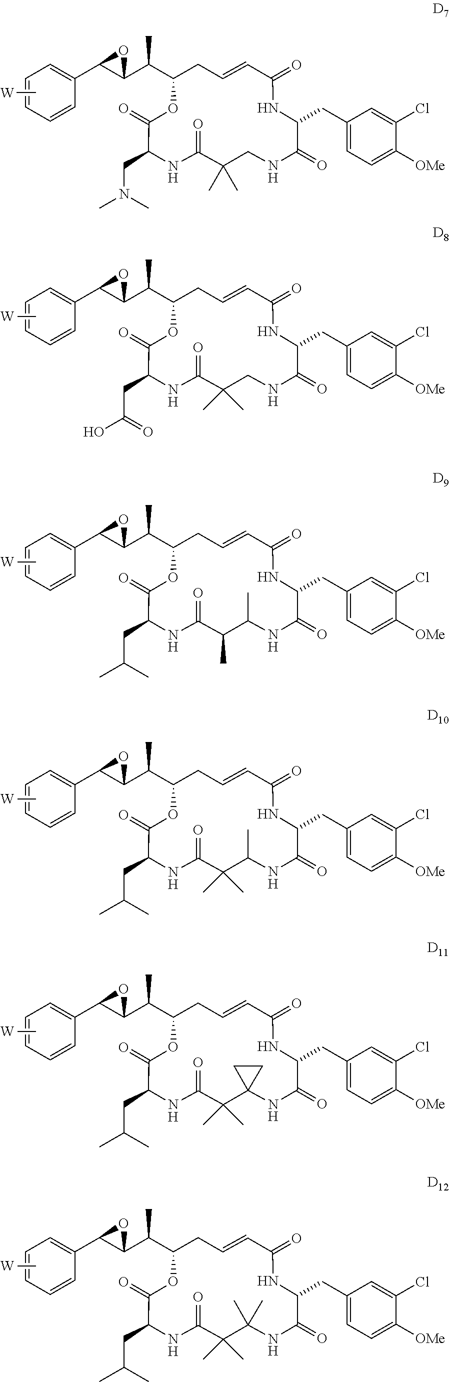

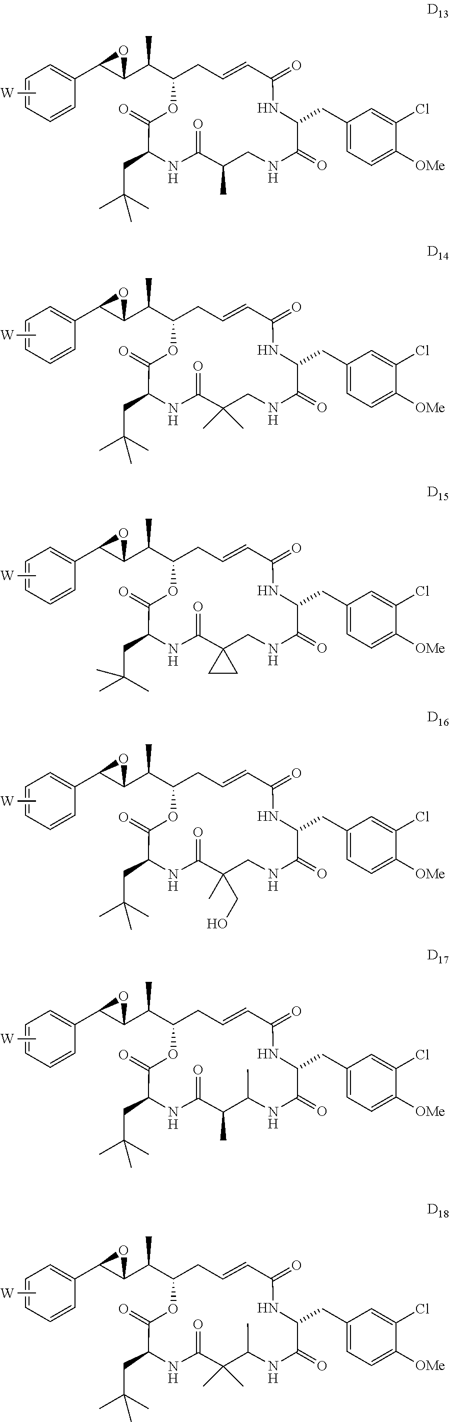

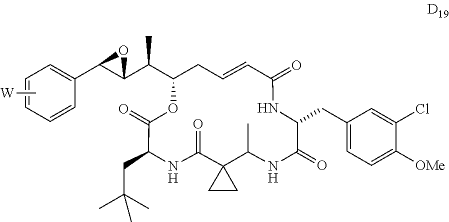

wherein W represents
   (C₁-C₆)alkyl-NH(R₁₁), such as (CH₂)ₙNHR₁₁;
   (C₁-C₆)alkyl-OH, such as (CH₂)ₙOH;
   (C₁-C₆)alkyl-SH, such as (CH₂)ₙSH;
   CO₂H or C(═O)NH₂;
   (C₁-C₆)alkyl-CO₂H or (C₁-C₆)alkyl-C(═O)NH₂; or
   (C₁-C₆)alkyl-N₃.
W is positioned in an ortho (o), meta (m) or para (p) position of the phenyl nucleus;
R₁₁ represents, independently of each other, a hydrogen atom or a group (C₁-C₆)alkyl, for instance a hydrogen atom or a methyl group;
n represents an integer ranging from 1 to 6;
   d: day; DAR: drug-to-antibody ratio (D1 refers to an ADC with a DAR of 1, D2 to an ADC with a DAR of 2, etc.); DBCO: dibenzylcyclooctyne; DCC: N,N′-dicyclohexylcarbodiimide; DCM: dichloromethane; DIEA: N,N-diisopropylethylamine; DMA: dimethylacetamide; DMAP: 4-(dimethylamino)pyridine; DMEM: Dulbecco's Modified Eagle Medium; DMEM/F12: Dulbecco's Modified Eagle Medium Nutrient Mixture F-12; DMF: dimethylformamide; DMSO: dimethylsulfoxide; DPBS: Dulbecco's phosphate-buffered saline; DSC: N,N′-disuccinimidyl carbonate; EDC: 1-(3-dimethylaminopropyl)-3-ethylcarbodiimide; EDTA: ethylenediaminetetraacetic acid; EEDQ: N-ethoxycarbonyl-2-ethoxy-1,2-dihydroquinoline; ELSD: evaporating light scattering detector; eq.: equivalent; ES: electrospray; EtOAc: ethyl acetate; Et₂O: diethyl ether; Ex.: example; FCS: foetal calf serum; Fmoc: 9-fluorenylmethoxycarbonyl; GI: electroinductive group; h: hour; H₂O: water; Hal: halogen; HATU: 1-[bis(dimethylamino)methylene]-1H-1,2,3-triazolo[4,5-b]pyridinium 3-oxid hexafluorophosphate; HCl: chlorohydric acid; HEPES: 4-(2-hydroxyethyl)-1-piperazineethanesulfonic acid; HIC: hydrophobic interaction chromatography; HOAt: 1-hydroxy-7-azabenzotriazole; HOBt: 1-hydroxy-benzotriazole; HPLC: high performance liquid chromatography; HRMS: high resolution mass spectrometry; IC₅₀: median inhibitory concentration; i.e.: id est meaning that is; IEC: ion exchange chromatography; iPrOH: 2-propanol; iPr₂O: diisopropyl ether; i.v.: intravenous; MeCN: acetonitrile; MeOH: methanol; MeTHF: 2-methyl-tetrahydrofuran; MFCO: 1-fluoro-2-cyclooctyne-1-carboxylic acid; MgSO₄: magnesium sulfate; min: minute; MsCl: methanesulfonyl chloride; MTBE: methyl tert-butyl ether; MTD: maximum tolerated dose; NaH: sodium hydride; NaCl: sodium chloride; NaHCO₃: sodium hydrogen carbonate; n.d.: not determined; NHS: N-hydroxysuccinimide; NMP: 1-methyl-2-pyrrolidone; NMR: nuclear magnetic resonance; PABA: para-aminobenzyl alcohol; PBS: phosphate-buffered saline; PEG: polyethylene glycol; PNGase F: Peptide-N-Glycosidase F; ppm: parts per million; PR: partial response; QS: quantum satis meaning the amount what is needed; Q-Tof: quadrupole time-of-flight; quant.: quantitative yield; RCG: reactive chemical group; RT: room temperature; sat.: saturated; s.c.: subcutaneously; SCID: severe combined immunodeficiency; SEC: size exclusion chromatography; T3P: propylphosphonic anhydride; TBAF: tetrabutylammonium fluoride; TFA: trifluoroacetic acid; TFS: tumor-free survivor; THF: tetrahydrofuran; TLC: thin layer chromatography; t_{1/2}: half-life; t_{R}: retention time; UPLC: Ultra Performance Liquid Chromatography; UV: ultra-violet.

### BRIEF DESCRIPTION OF THE FIGURES

FIG. 1: In vivo efficacy of Ex. 6 against MDA-MB-231 xenograft in SCID mice

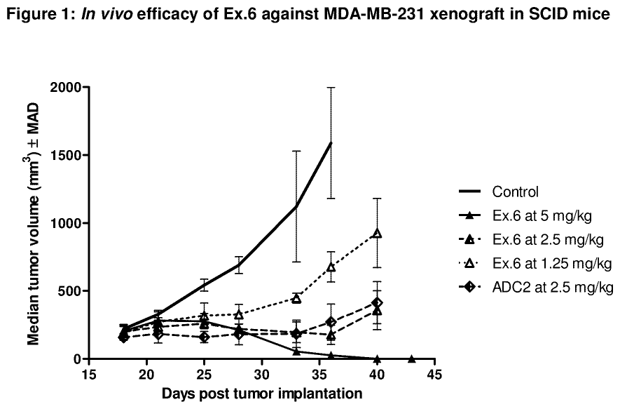

FIG. 2: In vivo efficacy of Ex. 16, Ex. 19, Ex. 23 and Ex. 32 against MDA-MB-231 xenograft in SCID mice at 2.5 mg/kg

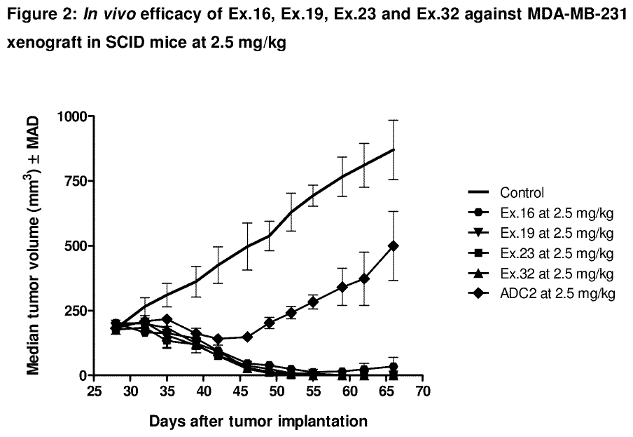

FIG. 3: In vivo efficacy of Ex. 16, Ex. 19, Ex. 23 and Ex. 32 against MDA-MB-231 xenograft in SCID mice at 1.25 mg/kg

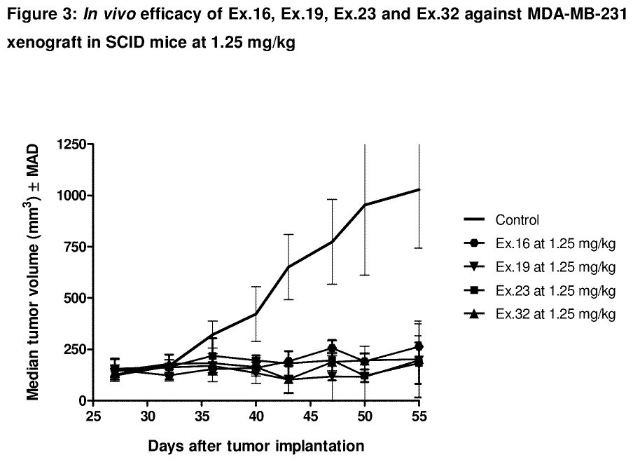

FIG. 4: In vivo efficacy of Ex. 26 against MDA-MB-231 xenograft in SCID mice

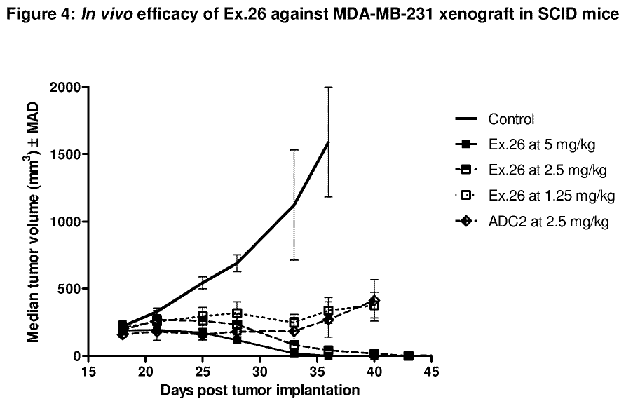

FIG. 5: In vivo efficacy of Ex. 29 against MDA-MB-231 xenograft in SCID mice

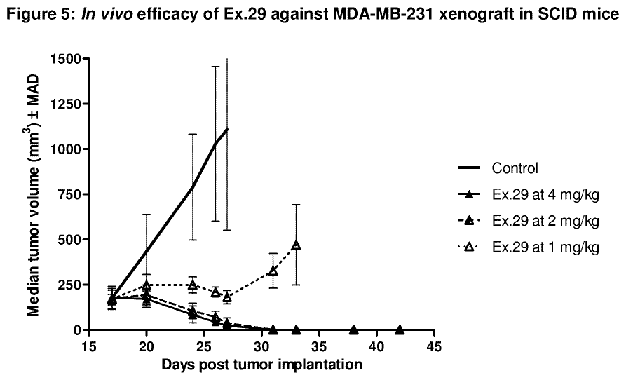

FIG. 6: In vivo efficacy of Ex. 35 against MDA-MB-231 xenograft in SCID mice

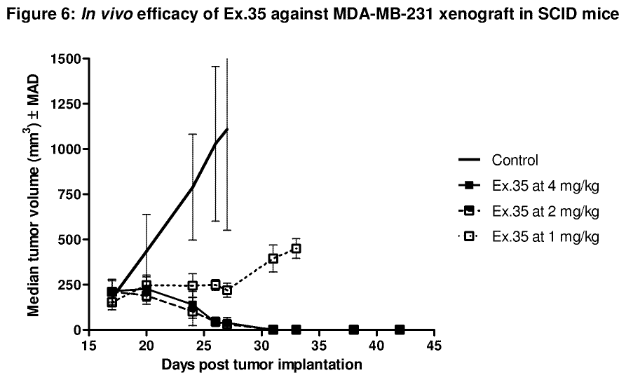

FIG. 7: In vivo efficacy of Ex. 41 against MDA-MB-231 xenograft in SCID mice

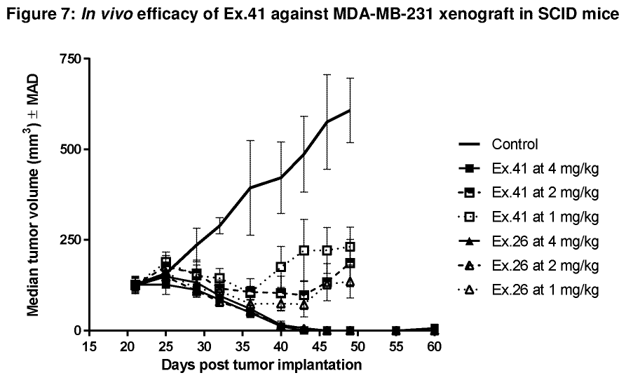

### DETAILED DESCRIPTION OF THE PRESENT DISCLOSURE

The disclosure relates to new peptidic compounds, chosen from compounds of formula (I):

RCG1-L-P (I)

wherein
RCG1 represents a reactive chemical group that is reactive towards a chemical group present on a polypeptide such as an antibody;
L represents a linker of formula (II) as defined below;
P represents a hydrogen atom, —OH or an activated O.

Examples of activated O that may be mentioned include

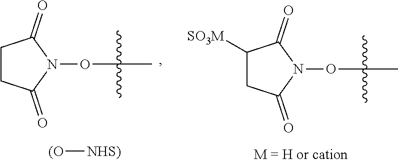

where cation represents for example sodium, potassium or cesium; or,

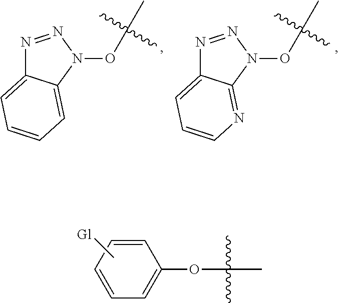

group in which GI represents at least one electroinductive group such as —NO₂ or a halogen atom (-Hal), for instance a fluorine atom (—F). They may be, for example, the following groups:

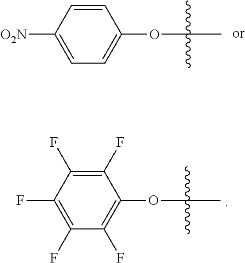 [SMILES: *Oc1ccc([N+](=O)[O-])cc1 ; "or" ; *Oc1c(F)c(F)c(F)c(F)c1F]

The linker L which is present in formula (I) is of formula (II):

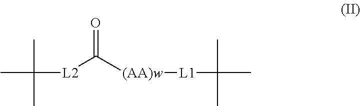

wherein:
L1 is of formula (III):

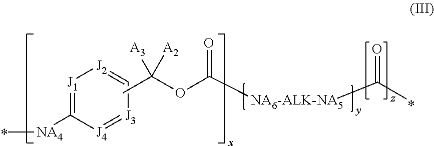

wherein:
   when P represents a hydrogen atom, then x=0 or 1 and y=1 and z=0;
   when P represents —OH, then x=y=z=0;
   when P represents an activated O, then x=1 and y=z=0, or x=y=z=1;
   J₁, J₂, J₃ and J₄ are chosen, independently of each other, from CA₁ and N;
   ALK represents a (C₁-C₁₂)alkylene group, for instance (C₁-C₆)alkylene, such as of the form —(CH₂)ₙ—, n being an integer ranging from 1 to 12 and for example ranging from 1 to 6;
   A₁, A₂, A₃, A₄, A₅, and A₆ represent, independently of each other, a hydrogen atom or a (C₁-C₆)alkyl group, such as a hydrogen atom or a methyl group.
   (AA)w represents a sequence of w substituted AAₛ or non-substituted amino acids AAₙₛ connected together via peptide bonds wherein substituted AAₛ or non-substituted AAₙₛ is defined below;
   w represents an integer ranging from 1 to 12, for instance from 1 to 6, such as 2 or 3;
if (AA)w contains at least one substituted amino acid AAₛ, then L2 represents: a single bond, a (C₁-C₆)alkyl group, a (C₁-C₆)alkyl-(OCH₂CH₂)ᵢ group, a (C₁-C₆)alkyl-(OCH₂CH₂)ᵢ—O(C₁-C₆)alkyl group, a (CH₂CH₂O)ᵢ(C₁-C₆)alkyl group, a CH(SO₃H)—(C₁-C₆)alkyl group, a (C₁-C₆)alkyl-CH(SO₃H) group, a (C₁-C₆)alkyl-cyclohexyl group, a C(═O)—(C₁-C₆)alkyl group, a C(═O)—(C₁-C₆)alkyl-(OCH₂CH₂)ᵢ group, a C(═O)—(C₁-C₆)alkyl-(OCH₂CH₂)ᵢ—O(C₁-C₆)alkyl group, a C(═O)—(CH₂CH₂O)ᵢ(C₁-C₆)alkyl group, a C(═O)—CH(SO₃H)—(C₁-C₆)alkyl group, a C(═O)—(C₁-C₆)alkyl-CH(SO₃H) group, a C(═O)—(C₁-C₆)alkyl-cyclohexyl group, a NA₈-(C₁-C₆)alkyl group, a NA₈-(C₁-C₆)alkyl-(OCH₂CH₂)ᵢ group, a NA₈-(C₁-C₆)alkyl-(OCH₂CH₂)ᵢ—O(C₁-C₆)alkyl group, a NA₈-(CH₂CH₂O)ᵢ(C₁-C₆)alkyl group, a NA₈-(C₁-C₆)alkyl-CH(SO₃H) group, a C(═O)—NA₈-(C₁-C₆)alkyl group, a C(═O)—NA₈-(C₁-C₆)alkyl-(OCH₂CH₂)ᵢ group, a C(═O)—NA₈-(C₁-C₆)alkyl-(OCH₂CH₂)ᵢ—O(C₁-C₆)alkyl group, a C(═O)—NA₈-(CH₂CH₂O)ᵢ(C₁-C₆)alkyl group, a C(═O)—NA₈-(C₁-C₆)alkyl-CH(SO₃H) group, a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ-NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ-NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ-NA₇-(C₁-C₆)alkyl group;
if (AA)w represents a sequence of w non-substituted amino acids AAₙS, then L2 represents:
   a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ-NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ-NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ-NA₇-(C₁-C₆)alkyl group;
A₇ representing a straight or branched, saturated or unsaturated, optionally substituted C₁-C₁₆₀ hydrocarbon chain wherein optionally at least one methylene unit is independently replaced by —NHCO—, —N(alkyl)C(═O)—, —C(═O)NH—, —C(═O)N(alkyl)-, —OC(═O)—, —C(═O)O—, —OC(═O)O—, —CH(OH)—, —CH(SO₃H)—, —O—, —C(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂; being understood that each A₇ comprising a SO₃H function can be under salt forms such as alkali metal salts, for instance sodium salts (SO₃^{- +}Na).
A₈ representing a hydrogen atom or a (C₁-C₆)alkyl group, for instance a hydrogen atom or a methyl group;
i representing an integer ranging from 1 to 50, such as ranging from 1 to 35.

According to a particular embodiment, the disclosure relates to compounds of formula (I):

RCG1-L-P (i)

wherein
RCG1 represents a reactive chemical group that is reactive towards a chemical group present on a polypeptide such as an antibody;
P represents a hydrogen atom, —OH or an activated 0;
L represents a linker of formula (II):

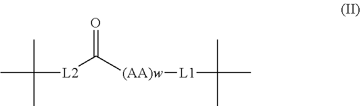

wherein:
L1 is of formula (III):

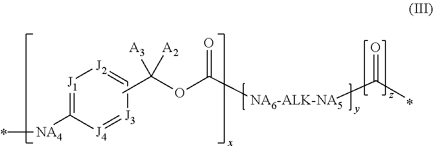

wherein:
   when P represents a hydrogen atom, then x=0 or 1 and y=1 and z=0;
   when P represents —OH, then x=y=z=0;
   when P represents an activated O, then x=1 and y=z=0, or x=y=z=1;
   J₁, J₂, J₃ and J₄ are chosen, independently of each other, from CA₁ and N;
   ALK represents a (C₁-C₁₂)alkylene group, for instance (C₁-C₆)alkylene, such as of the form —(CH₂)ₙ—, n being an integer ranging from 1 to 12 and for example ranging from 1 to 6;
   A₁, A₂, A₃, A₄, A₅, and A₆ represent, independently of each other, a hydrogen atom or a (C₁-C₆)alkyl group, such as a hydrogen atom or a methyl group.
   (AA)w represents a sequence of w substituted AAₛ or non-substituted amino acids AAₙₛ connected together via peptide bonds;
   w represents an integer ranging from 1 to 12, for instance from 1 to 6, such as 2 or 3;
if (AA)w contains at least one substituted amino acid AAₛ, then L2 represents a single bond, a (C₁-C₆)alkyl group, a (C₁-C₆)alkyl-(OCH₂CH₂)ᵢ group, a (C₁-C₆)alkyl-(OCH₂CH₂)ᵢ—O(C₁-C₆)alkyl group, a (CH₂CH₂O)ᵢ(C₁-C₆)alkyl group, a CH(SO₃H)—(C₁-C₆)alkyl group, a (C₁-C₆)alkyl-CH(SO₃H) group, a (C₁-C₆)alkyl-cyclohexyl group, a C(═O)—(C₁-C₆)alkyl group, a C(═O)—(C₁-C₆)alkyl-(OCH₂CH₂)ᵢ group, a C(═O)—(C₁-C₆)alkyl-(OCH₂CH₂)ᵢ—O(C₁-C₆)alkyl group, a C(═O)—(CH₂CH₂O)ᵢ(C₁-C₆)alkyl group, a C(═O)—CH(SO₃H)—(C₁-C₆)alkyl group, a C(═O)—(C₁-C₆)alkyl-CH(SO₃H) group, a C(═O)—(C₁-C₆)alkyl-cyclohexyl group, a NA₈-(C₁-C₆)alkyl group, a NA₈-(C₁-C₆)alkyl-(OCH₂CH₂)ᵢ group, a NA₈-(C₁-C₆)alkyl-(OCH₂CH₂)ᵢ—O(C₁-C₆)alkyl group, a NA₈-(CH₂CH₂O)ᵢ(C₁-C₆)alkyl group, a NA₈-(C₁-C₆)alkyl-CH(SO₃H) group, a C(═O)—NA₈-(C₁-C₆)alkyl group, a C(═O)—NA₈-(C₁-C₆)alkyl-(OCH₂CH₂)ᵢ group, a C(═O)—NA₈-(C₁-C₆)alkyl-(OCH₂CH₂)ᵢ—O(C₁-C₆)alkyl group, a C(═O)—NA₈-(CH₂CH₂O)ᵢ(C₁-C₆)alkyl group, a C(═O)—NA₈-(C₁-C₆)alkyl-CH(SO₃H) group, a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ-NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ-NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ-NA₇-(C₁-C₆)alkyl group;
if (AA)w represents a sequence of w non-substituted amino acids AAₙₛ, then L2 represents a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ-NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ-NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ-NA₇-(C₁-C₆)alkyl group;
A₇ representing a straight or branched, saturated or unsaturated, optionally substituted C₁-C₁₆₀ hydrocarbon chain wherein optionally at least one methylene unit is independently replaced by —NHC(═O)—, —N(alkyl)C(═O)—, —C(═O)NH—, —C(═O)N(alkyl)-, —OC(═O)—, —C(═O)O—, —OC(═O)O—, —CH(OH)—, —CH(SO₃H)—, —O—, —C(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, being understood that each A₇ comprising a SO₃H function can be under salt forms such as alkali metal salts, for instance sodium salts (SO₃^{- +}Na);
A₈ representing a hydrogen atom or a (C₁-C₆)alkyl group, for instance a hydrogen atom or a methyl group;
i representing an integer ranging from 1 to 50, for instance ranging from 1 to 35,
a non-substituted amino acid AAₙS denoting natural or non-natural amino acid, of configuration D or L, identical to or derived from: alanine (Ala), β-alanine, γ-aminobutyric acid, 2-amino-2-cyclohexylacetic acid, 2-amino-2-phenylacetic acid, arginine (Arg), asparagine (Asn), aspartic acid (Asp), citrulline (Cit), cysteine (Cys), α,α-dimethyl-γ-aminobutyric acid, β,β-dimethyl-γ-aminobutyric acid, glutamine (Gin), glutamic acid (Glu), glycine (Gly), homo-cysteine, selenocysteine, homo-selenocysteine, histidine (His), isoleucine (Ile), leucine (Leu), lysine (Lys), ε-acetyl-lysine (AcLys), methionine (Met), ornithine (Orn), phenylalanine (Phe), proline (Pro), serine (Ser), threonine (Thr), tryptophan (Trp), tyrosine (Tyr), and valine (Val).

According to another particular embodiment, the disclosure relates to compounds of formula (I):

RCG1-L-P (I)

wherein
RCG1 represents a reactive chemical group that is reactive towards a chemical group present on a polypeptide such as an antibody;
P represents a hydrogen atom, —OH or an activated O;
L represents a linker of formula (II):

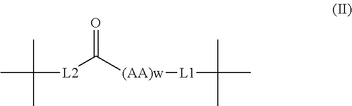

wherein:
L1 is of formula (III):

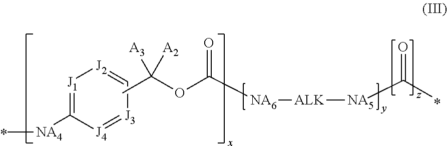

wherein:
   when P represents a hydrogen atom, then x=0 or 1 and y=1 and z=0;
   when P represents —OH, then x=y=z=0;
   when P represents an activated O, then x=1 and y=z=0, or x=y=z=1;
   J₁, J₂, J₃ and J₄ are chosen, independently of each other, from CA₁ and N;
   ALK represents a (C₁-C₁₂)alkylene group, for instance (C₁-C₆)alkylene, such as of the form —(CH₂)ₙ—, n being an integer ranging from 1 to 12 and for example ranging from 1 to 6;
   A₁, A₂, A₃, A₄, A₅, and A₆ represent, independently of each other, a hydrogen atom or a (C₁-C₆)alkyl group, such as a hydrogen atom or a methyl group.
   (AA)w represents a sequence of w substituted AAₛ or non-substituted amino acids AAₙₛ connected together via peptide bonds;
   w represents an integer ranging from 1 to 12, for instance from 1 to 6, such as 2 or 3;
if (AA)w contains at least one substituted amino acid AAₛ, then L2 represents a single bond, a (C₁-C₆)alkyl group, a (C₁-C₆)alkyl-(OCH₂CH₂)ᵢ group, a (C₁-C₆)alkyl-(OCH₂CH₂)ᵢ—O(C₁-C₆)alkyl group, a (CH₂CH₂O)ᵢ(C₁-C₆)alkyl group, a CH(SO₃H)—(C₁-C₆)alkyl group, a (C₁-C₆)alkyl-CH(SO₃H) group, a (C₁-C₆)alkyl-cyclohexyl group, a C(═O)—(C₁-C₆)alkyl group, a C(═O)—(C₁-C₆)alkyl-(OCH₂CH₂)ᵢ group, a C(═O)—(C₁-C₆)alkyl-(OCH₂CH₂)ᵢ—O(C₁-C₆)alkyl group, a C(═O)—(CH₂CH₂O)ᵢ(C₁-C₆)alkyl group, a C(═O)—CH(SO₃H)—(C₁-C₆)alkyl group, a C(═O)—(C₁-C₆)alkyl-CH(SO₃H) group, a C(═O)—(C₁-C₆)alkyl-cyclohexyl group, a NA₈-(C₁-C₆)alkyl group, a NA₈-(C₁-C₆)alkyl-(OCH₂CH₂)ᵢ group, a NA₈-(C₁-C₆)alkyl-(OCH₂CH₂)ᵢ—O(C₁-C₆)alkyl group, a NA₈-(CH₂CH₂O)ᵢ(C₁-C₆)alkyl group, a NA₈-(C₁-C₆)alkyl-CH(SO₃H) group, a C(═O)—NA₈-(C₁-C₆)alkyl group, a C(═O)—NA₈-(C₁-C₆)alkyl-(OCH₂CH₂)ᵢ group, a C(═O)—NA₈-(C₁-C₆)alkyl-(OCH₂CH₂)ᵢ—O(C₁-C₆)alkyl group, a C(═O)—NA₈-(CH₂CH₂O)ᵢ(C₁-C₆)alkyl group, a C(═O)—NA₈-(C₁-C₆)alkyl-CH(SO₃H) group, a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ-NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ-NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ-NA₇-(C₁-C₆)alkyl group;
if (AA)w represents a sequence of w non-substituted amino acids AAₙₛ, then L2 represents a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ-NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ-NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ—NA₇-(C₁-C₆)alkyl group;
A₇ representing a straight or branched, saturated or unsaturated, optionally substituted C₁-C₁₆₀ hydrocarbon chain wherein optionally at least one methylene unit is independently replaced by —NHC(═O)—, —N(alkyl)C(═O)—, —C(═O)NH—, —C(═O)N(alkyl)-, —OC(═O)—, —C(═O)O—, —OC(═O)O—, —CH(OH)—, —CH(SO₃H)—, —O—, —C(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, being understood that each A₇ comprising a SO₃H function can be under salt forms such as alkali metal salts, for instance sodium salts (SO₃^{- +}Na);
A₈ representing a hydrogen atom or a (C₁-C₆)alkyl group, for instance a hydrogen atom or a methyl group;
i representing an integer ranging from 1 to 50, for instance ranging from 1 to 35,
   wherein said substituted amino acids AAₛ have the formula (VI):

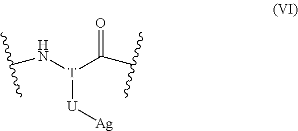

wherein:
T represents a saturated or unsaturated, linear or branched, (C₁-C₈) trivalent alkyl group, preferably T is

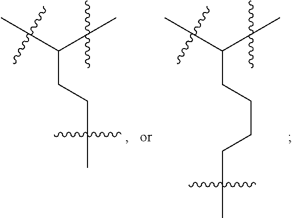 [SMILES: *CCC(*)* ; *CCCCC(*)*]

U group represents a single bond, —NHC(═O)—, —N(alkyl)C(═O)—, —C(═O)NH—, —C(═O)N(alkyl)-, —NHC(═O)NH—, —NHC(═NH)NH—, —OC(═O)—, —C(═O)O—, —OC(═O)O—, —S—, —Se—, —O—, —NH—, —N(alkyl)-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, or —O—P(═O)(OH)—O, such as U group represents —NH—C(═O)—, or —C(═O)NH—;
A₉ represents a straight or branched, saturated or unsaturated, optionally substituted C₁-C₁₆₀ hydrocarbon chain wherein optionally at least one methylene unit is independently replaced by —NH—C(═O)—, —N(alkyl)C(═O)—, —C(═O)NH—, —C(═O)N(alkyl)-, —NHC(═O)NH—, —NHC(═NH)NH—, —OC(═O)—, —C(═O)O—, —OC(═O)O—, —CH(OH)—, —CH(SO₃H)—, —CH(Oalkyl)-, —CHF—, —CF₂—, —S—, —Se—, —O—, —NH—, —N(alkyl)-, —N⁺H(alkyl)-, —N⁺(alkyl)₂-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, for instance A₉ is —[(CH₂)₂—O]₄—CH₃, —[(CH₂)₂—O]₂₄—CH₃,

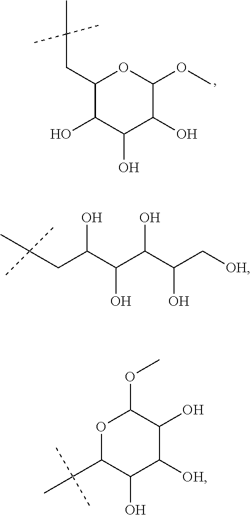

or —[(CH₂)₂—O)]₄—(CH₂)₂—C(═O)—NH—(CH₂)₂—SO₃H;
being understood that each A₉ comprising a SO₃H function can be under salt forms such as alkali metal salts, for instance sodium salts (SO₃^{- +}Na).

According to another particular embodiment, the disclosure relates to compounds of formula (I):

RCG1-L-P (I)

wherein
RCG1 represents a reactive chemical group that is reactive towards a chemical group present on a polypeptide such as an antibody;
P represents a hydrogen atom, —OH or an activated 0;
L represents a linker of formula (II):

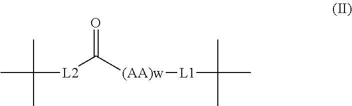

wherein:
L1 is of formula (III):

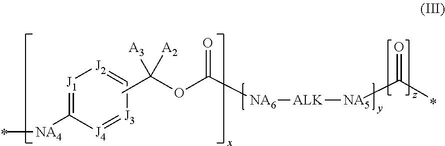

wherein:
   when P represents a hydrogen atom, then x=0 or 1 and y=1 and z=0;
   when P represents —OH, then x=y=z=0;
   when P represents an activated O, then x=1 and y=z=0, or x=y=z=1;
   J₁, J₂, J₃ and J₄ are chosen, independently of each other, from CA₁ and N;
   ALK represents a (C₁-C₁₂)alkylene group, for instance (C₁-C₆)alkylene, such as of the form —(CH₂)ₙ—, n being an integer ranging from 1 to 12 and for example ranging from 1 to 6;
   A₁, A₂, A₃, A₄, A₅, and A₆ represent, independently of each other, a hydrogen atom or a (C₁-C₆)alkyl group, such as a hydrogen atom or a methyl group.
   (AA)w represents a sequence of w substituted AAₛ or non-substituted amino acids AAₙₛ connected together via peptide bonds;
   w represents an integer ranging from 1 to 12, for instance from 1 to 6, such as 2 or 3;
if (AA)w contains at least one substituted amino acid AAₛ, then L2 represents a single bond, a (C₁-C₆)alkyl group, a (C₁-C₆)alkyl-(OCH₂CH₂)ᵢ group, a (C₁-C₆)alkyl-(OCH₂CH₂)ᵢ—O(C₁-C₆)alkyl group, a (CH₂CH₂O)ᵢ(C₁-C₆)alkyl group, a CH(SO₃H)—(C₁-C₆)alkyl group, a (C₁-C₆)alkyl-CH(SO₃H) group, a (C₁-C₆)alkyl-cyclohexyl group, a C(═O)—(C₁-C₆)alkyl group, a C(═O)—(C₁-C₆)alkyl-(OCH₂CH₂)ᵢ group, a C(═O)—(C₁-C₆)alkyl-(OCH₂CH₂)ᵢ—O(C₁-C₆)alkyl group, a C(═O)—(CH₂CH₂O)ᵢ(C₁-C₆)alkyl group, a C(═O)—CH(SO₃H)—(C₁-C₆)alkyl group, a C(═O)—(C₁-C₆)alkyl-CH(SO₃H) group, a C(═O)—(C₁-C₆)alkyl-cyclohexyl group, a NA₈-(C₁-C₆)alkyl group, a NA₈-(C₁-C₆)alkyl-(OCH₂CH₂)ᵢ group, a NA₈-(C₁-C₆)alkyl-(OCH₂CH₂)ᵢ—O(C₁-C₆)alkyl group, a NA₈-(CH₂CH₂O)ᵢ(C₁-C₆)alkyl group, a NA₈-(C₁-C₆)alkyl-CH(SO₃H) group, a C(═O)—NA₈-(C₁-C₆)alkyl group, a C(═O)—NA₈-(C₁-C₆)alkyl-(OCH₂CH₂)ᵢ group, a C(═O)—NA₈-(C₁-C₆)alkyl-(OCH₂CH₂)ᵢ—O(C₁-C₆)alkyl group, a C(═O)—NA₈-(CH₂CH₂O)ᵢ(C₁-C₆)alkyl group, a C(═O)—NA₈-(C₁-C₆)alkyl-CH(SO₃H) group, a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ—NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ—NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ—NA₇-(C₁-C₆)alkyl group;
if (AA)w represents a sequence of w non-substituted amino acids AAₙₛ, then L2 represents a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ—NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ—NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ—NA₇-(C₁-C₆)alkyl group;
A₇ representing a straight or branched, saturated or unsaturated, optionally substituted C₁-C₁₆₀ hydrocarbon chain wherein optionally at least one methylene unit is independently replaced by —NHC(═O)—, —N(alkyl)C(═O)—, —C(═O)NH—, —C(═O)N(alkyl)-, —OC(═O)—, —C(═O)O—, —OC(═O)O—, —CH(OH)—, —CH(SO₃H)—, —O—, —C(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, being understood that each A₇ comprising a SO₃H function can be under salt forms such as alkali metal salts, for instance sodium salts (SO₃^{- +}Na);
A₈ representing a hydrogen atom or a (C₁-C₆)alkyl group, for instance a hydrogen atom or a methyl group;
i representing an integer ranging from 1 to 50, for instance ranging from 1 to 35,
   wherein said substituted amino acids AAₛ have the formula (VI):

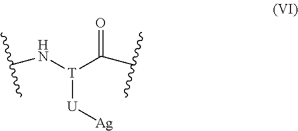

wherein:
T represents a saturated or unsaturated, linear or branched, (C₁-C₈) trivalent alkyl group, preferably T is

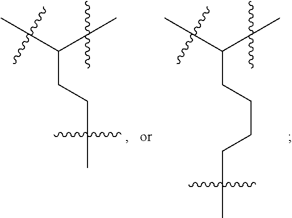 [SMILES: *CCC(*)* ; *CCCCC(*)*]

U group represents a single bond, —NHC(═O)—, —N(alkyl)C(═O)—, —C(═O)NH—, —C(═O)N(alkyl)-, —NHC(═O)NH—, —NHC(═NH)NH—, —OC(═O)—, —C(═O)O—, —OC(═O)O—, —S—, —Se—, —O—, —NH—, —N(alkyl)-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, or —O—P(═O)(OH)—O, such as U group represents —NH—C(═O)—, or —C(═O)NH—;
A₉ represents a straight or branched, saturated or unsaturated, optionally substituted C₁-C₁₆₀ hydrocarbon chain wherein optionally at least one methylene unit is independently replaced by —NH—C(═O)—, —N(alkyl)C(═O)—, —C(═O)NH—, —C(═O)N(alkyl)-, —NHC(═NH)NH—, —OC(═O)—, —C(═O)O—, —OC(═O)O—, —CH(OH)—, —CH(SO₃H)—, —CH(Oalkyl)-, —CHF—, —CF₂—, —S—, —Se—, —O—, —NH—, —N(alkyl)-, —N⁺H(alkyl)-, —N⁺(alkyl)₂-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, for instance A₉ is —[(CH₂)₂—O]₄—CH₃, —[(CH₂)₂—O]₂₄—CH₃,

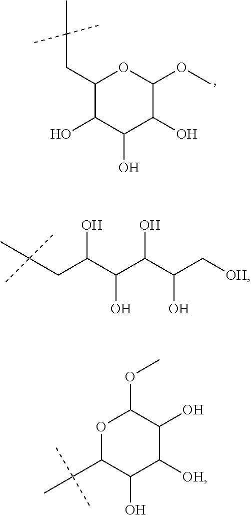

or —[(CH₂)₂—O)]₄—(CH₂)₂—C(═O)—NH—(CH₂)₂—SO₃H;
being understood that each A₉ comprising a SO₃H function can be under salt forms such as alkali metal salts, for instance sodium salts (SO₃^{- +}Na).

According to another particular embodiment, the disclosure relates to compounds of formula (I):

RCG1-L-P (I)

wherein
RCG1 represents a reactive chemical group that is reactive towards a chemical group present on a polypeptide such as an antibody;
P represents a hydrogen atom, —OH or an activated 0;
L represents a linker of formula (II):

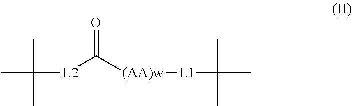

wherein:
L1 is of formula (III):

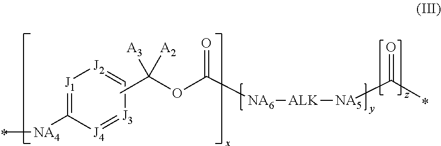

wherein:
   when P represents a hydrogen atom, then x=0 or 1 and y=1 and z=0;
   when P represents —OH, then x=y=z=0;
   when P represents an activated O, then x=1 and y=z=0, or x=y=z=1;
   J₁, J₂, J₃ and J₄ are chosen, independently of each other, from CA₁ and N;
   ALK represents a (C₁-C₁₂)alkylene group, for instance (C₁-C₆)alkylene, such as of the form —(CH₂)ₙ—, n being an integer ranging from 1 to 12 and for example ranging from 1 to 6;
   A₁, A₂, A₃, A₄, A₅, and A₆ represent, independently of each other, a hydrogen atom or a (C₁-C₆)alkyl group, such as a hydrogen atom or a methyl group.
   (AA)w represents a sequence of w substituted AAₛ or non-substituted amino acids AAₙS connected together via peptide bonds;
   w represents an integer ranging from 1 to 12, for instance from 1 to 6, such as 2 or 3;
if (AA)w contains at least one substituted amino acid AA_{S}, then L2 represents a single bond, a (C₁-C₆)alkyl group, a (C₁-C₆)alkyl-(OCH₂CH₂)ᵢ group, a (C₁-C₆)alkyl-(OCH₂CH₂)ᵢ—O(C₁-C₆)alkyl group, a (CH₂CH₂O)ᵢ(C₁-C₆)alkyl group, a CH(SO₃H)—(C₁-C₆)alkyl group, a (C₁-C₆)alkyl-CH(SO₃H) group, a (C₁-C₆)alkyl-cyclohexyl group, a C(═O)—(C₁-C₆)alkyl group, a C(═O)—(C₁-C₆)alkyl-(OCH₂CH₂)ᵢ group, a C(═O)—(C₁-C₆)alkyl-(OCH₂CH₂)ᵢ—O(C₁-C₆)alkyl group, a C(═O)—(CH₂CH₂O)ᵢ(C₁-C₆)alkyl group, a C(═O)—CH(SO₃H)—(C₁-C₆)alkyl group, a C(═O)—(C₁-C₆)alkyl-CH(SO₃H) group, a C(═O)—(C₁-C₆)alkyl-cyclohexyl group, a NA₈-(C₁-C₆)alkyl group, a NA₈-(C₁-C₆)alkyl-(OCH₂CH₂)ᵢ group, a NA₈-(C₁-C₆)alkyl-(OCH₂CH₂)ᵢ—O(C₁-C₆)alkyl group, a NA₈-(CH₂CH₂O)ᵢ(C₁-C₆)alkyl group, a NA₈-(C₁-C₆)alkyl-CH(SO₃H) group, a C(═O)—NA₈-(C₁-C₆)alkyl group, a C(═O)—NA₈-(C₁-C₆)alkyl-(OCH₂CH₂)ᵢ group, a C(═O)—NA₈-(C₁-C₆)alkyl-(OCH₂CH₂)ᵢ—O(C₁-C₆)alkyl group, a C(═O)—NA₈-(CH₂CH₂O)ᵢ(C₁-C₆)alkyl group, a C(═O)—NA₈-(C₁-C₆)alkyl-CH(SO₃H) group, a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ—NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ—NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈—(C₁-C₆)alkyl-(OCH₂CH₂)ᵢ—NA₇-(C₁-C₆)alkyl group;
if (AA)w represents a sequence of w non-substituted amino acids AAₙS, then L2 represents a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ—NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ—NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ—NA₇-(C₁-C₆)alkyl group;
A₇ representing a straight or branched, saturated or unsaturated, optionally substituted C₁-C₁₆₀ hydrocarbon chain wherein optionally at least one methylene unit is independently replaced by —NHC(═O)—, —N(alkyl)C(═O)—, —C(═O)NH—, —C(═O)N(alkyl)-, —OC(═O)—, —C(═O)O—, —OC(═O)O—, —CH(OH)—, —CH(SO₃H)—, —O—, —C(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, being understood that each A₇ comprising a SO₃H function can be under salt forms such as alkali metal salts, for instance sodium salts (SO₃^{- +}Na);
A₈ representing a hydrogen atom or a (C₁-C₆)alkyl group, for instance a hydrogen atom or a methyl group;
i representing an integer ranging from 1 to 50, for instance ranging from 1 to 35,
   wherein said substituted amino acids AAₛ have the formula (VI):

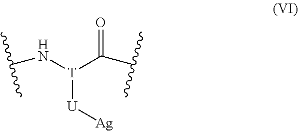

wherein:
T represents a saturated or unsaturated, linear or branched, (C₁-C₈) trivalent alkyl group, preferably T is

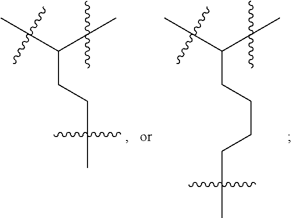 [SMILES: *CCC(*)* ; *CCCCC(*)*]

U group represents a single bond, —NHC(═O)—, —N(alkyl)C(═O)—, —C(═O)NH—, —C(═O)N(alkyl)-, —NHC(═O)NH—, —NHC(═NH)NH—, —OC(═O)—, —C(═O)O—, —OC(═O)O—, —S—, —Se—, —O—, —NH—, —N(alkyl)-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, or —O—P(═O)(OH)—O, such as U group represents —NH—C(═O)—, or —C(═O)NH—;
A₉ represents a straight or branched, saturated or unsaturated, optionally substituted C₁-C₁₆₀ hydrocarbon chain wherein optionally at least one methylene unit is independently replaced by —NH—C(═O)—, —N(alkyl)C(═O)—, —C(═O)NH—, —C(═O)N(alkyl)-, —NHC(═NH)NH—, —OC(═O)—, —C(═O)O—, —OC(═O)O—, —CH(OH)—, —CH(SO₃H)—, —CH(Oalkyl)-, —CHF—, —CF₂—, —S—, —Se—, —O—, —N(alkyl)-, —N⁺H(alkyl)-, —N⁺(alkyl)₂-, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, for instance A₉ is —[(CH₂)₂—O]₄—CH₃, —[(CH₂)₂—O]₂₄—CH₃,

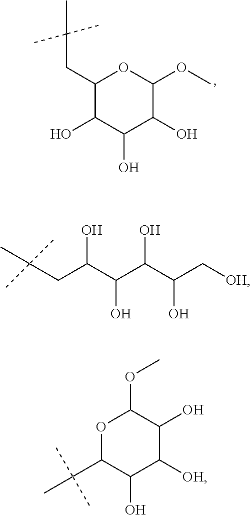

or —[(CH₂)₂—O)]₄—(CH₂)₂—C(═O)—NH—(CH₂)₂—SO₃H;
being understood that each A₉ comprising a SO₃H function can be under salt forms such as alkali metal salts, for instance sodium salts (SO₃^{- +}Na).

According to another particular embodiment, the disclosure relates to compounds of formula (I):

RCG1-L-P (I)

wherein
RCG1 represents a reactive chemical group that is reactive towards a chemical group present on a polypeptide such as an antibody;
P represents a hydrogen atom, —OH or an activated 0;
L represents a linker of formula (II):

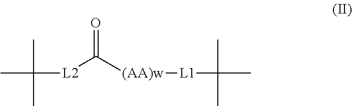

wherein:
L1 is of formula (III):

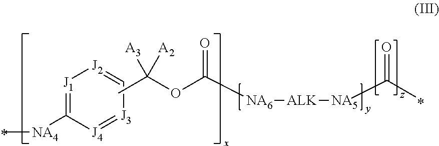

wherein:
   when P represents a hydrogen atom, then x=0 or 1 and y=1 and z=0;
   when P represents —OH, then x=y=z=0;
   when P represents an activated O, then x=1 and y=z=0, or x=y=z=1;
   J₁, J₂, J₃ and J₄ are chosen, independently of each other, from CA₁ and N;
   ALK represents a (C₁-C₁₂)alkylene group, for instance (C₁-C₆)alkylene, such as of the form —(CH₂)ₙ—, n being an integer ranging from 1 to 12 and for example ranging from 1 to 6;
   A₁, A₂, A₃, A₄, A₅, and A₆ represent, independently of each other, a hydrogen atom or a (C₁-C₆)alkyl group, such as a hydrogen atom or a methyl group.
   (AA)w represents a sequence of w substituted AAₛ or non-substituted amino acids AAₙS connected together via peptide bonds;
   w represents an integer ranging from 1 to 12, for instance from 1 to 6, such as 2 or 3;
if (AA)w contains at least one substituted amino acid AAₛ, then L2 represents a single bond, a (C₁-C₆)alkyl group, a (C₁-C₆)alkyl-(OCH₂CH₂)ᵢ group, a (C₁-C₆)alkyl-(OCH₂CH₂)ᵢ—O(C₁-C₆)alkyl group, a (CH₂CH₂O)ᵢ(C₁-C₆)alkyl group, a CH(SO₃H)—(C₁-C₆)alkyl group, a (C₁-C₆)alkyl-CH(SO₃H) group, a (C₁-C₆)alkyl-cyclohexyl group, a C(═O)—(C₁-C₆)alkyl group, a C(═O)—(C₁-C₆)alkyl-(OCH₂CH₂)ᵢ group, a C(═O)—(C₁-C₆)alkyl-(OCH₂CH₂)ᵢ—O(C₁-C₆)alkyl group, a C(═O)—(CH₂CH₂O)ᵢ(C₁-C₆)alkyl group, a C(═O)—CH(SO₃H)—(C₁-C₆)alkyl group, a C(═O)—(C₁-C₆)alkyl-CH(SO₃H) group, a C(═O)—(C₁-C₆)alkyl-cyclohexyl group, a NA₈-(C₁-C₆)alkyl group, a NA₈-(C₁-C₆)alkyl-(OCH₂CH₂)ᵢ group, a NA₈-(C₁-C₆)alkyl-(OCH₂CH₂)ᵢ—O(C₁-C₆)alkyl group, a NA₈-(CH₂CH₂O)ᵢ(C₁-C₆)alkyl group, a NA₈-(C₁-C₆)alkyl-CH(SO₃H) group, a C(═O)—NA₈-(C₁-C₆)alkyl group, a C(═O)—NA₈-(C₁-C₆)alkyl-(OCH₂CH₂)ᵢ group, a C(═O)—NA₈-(C₁-C₆)alkyl-(OCH₂CH₂)ᵢ—O(C₁-C₆)alkyl group, a C(═O)—NA₈-(CH₂CH₂O)ᵢ(C₁-C₆)alkyl group, a C(═O)—NA₈-(C₁-C₆)alkyl-CH(SO₃H) group, a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ—NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ—NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ—NA₇-(C₁-C₆)alkyl group;
if (AA)w represents a sequence of w non-substituted amino acids AAₙₛ, then L2 represents a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ—NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ—NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ—NA₇-(C₁-C₆)alkyl group;
A₇ representing a straight or branched, saturated or unsaturated, optionally substituted C₁-C₁₆₀ hydrocarbon chain wherein optionally at least one methylene unit is independently replaced by —NHC(═O)—, —N(alkyl)C(═O)—, —C(═O)NH—, —C(═O)N(alkyl)-, —OC(═O)—, —C(═O)O—, —OC(═O)O—, —CH(OH)—, —CH(SO₃H)—, —O—, —C(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, being understood that each A₇ comprising a SO₃H function can be under salt forms such as alkali metal salts, for instance sodium salts (SO₃^{- +}Na);
A₈ representing a hydrogen atom or a (C₁-C₆)alkyl group, for instance a hydrogen atom or a methyl group;
i representing an integer ranging from 1 to 50, for instance ranging from 1 to 35,
   wherein said substituted amino acids AAₛ have the formula (VI):

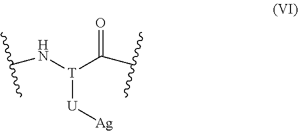

wherein:
T represents a saturated or unsaturated, linear or branched, (C₁-C₈) trivalent alkyl group, preferably T is

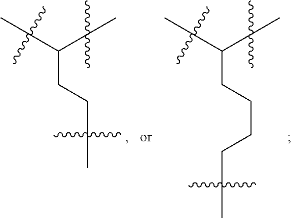 [SMILES: *CCC(*)* ; *CCCCC(*)*]

U group represents a single bond, —NHC(═O)—, —N(alkyl)C(═O)—, —C(═O)NH—, —C(═O)N(alkyl)-, —NHC(═O)NH—, —NHC(═NH)NH—, —OC(═O)—, —C(═O)O—, —OC(═O)O—, —S—, —Se—, —O—, —NH—, —N(alkyl)-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, or —O—P(═O)(OH)—O, such as U group represents —NH—C(═O)—, or —C(═O)NH—;
A₉ represents a straight or branched, saturated or unsaturated, optionally substituted C₁-C₁₆₀ hydrocarbon chain wherein optionally at least one methylene unit is independently replaced by —NH—C(═O)—, —N(alkyl)C(═O)—, —C(═O)NH—, —C(═O)N(alkyl)-, —C(═O)O—, —OC(═O)O—, —CH(OH)—, —CH(SO₃H)—, —CH(Oalkyl)-, —CHF—, —CF₂—, —Se—, —O—, —N(alkyl)-, —N⁺H(alkyl)-, —N⁺(alkyl)₂-, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, for instance A₉ is —[(CH₂)₂—O]₄—CH₃, —[(CH₂)₂—O]₂₄—CH₃,

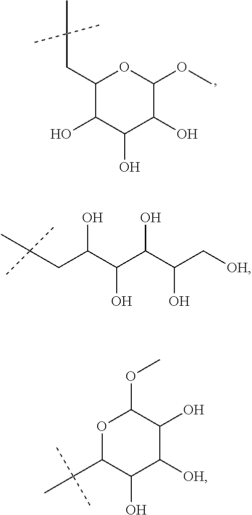

or —[(CH₂)₂—O)]₄—(CH₂)₂—C(═O)—NH—(CH₂)₂—SO₃H;
being understood that each A₉ comprising a SO₃H function can be under salt forms such as alkali metal salts, for instance sodium salts (SO₃^{- +}Na).

According to another particular embodiment, the disclosure relates to compounds of formula (I):

RCG1-L-P (I)

wherein
RCG1 represents a reactive chemical group that is reactive towards a chemical group present on a polypeptide such as an antibody;
P represents a hydrogen atom, —OH or an activated 0;
L represents a linker of formula (II):

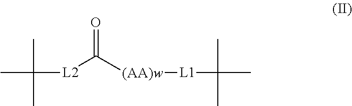

wherein:
L1 is of formula (III):

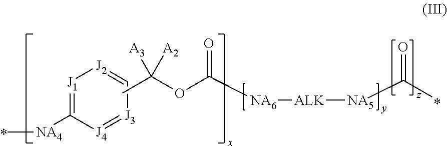

wherein:
   when P represents a hydrogen atom, then x=0 or 1 and y=1 and z=0;
   when P represents —OH, then x=y=z=0;
   when P represents an activated O, then x=1 and y=z=0, or x=y=z=1;
   J₁, J₂, J₃ and J₄ are chosen, independently of each other, from CA₁ and N;
   ALK represents a (C₁-C₁₂)alkylene group, for instance (C₁-C₆)alkylene, such as of the form —(CH₂)ₙ—, n being an integer ranging from 1 to 12 and for example ranging from 1 to 6;
   A₁, A₂, A₃, A₄, A₅, and A₆ represent, independently of each other, a hydrogen atom or a (C₁-C₆)alkyl group, such as a hydrogen atom or a methyl group.
   (AA)w represents a sequence of w substituted AAₛ or non-substituted amino acids AAₙₛ connected together via peptide bonds;
   w represents an integer ranging from 1 to 12, for instance from 1 to 6, such as 2 or 3;
   (AA)w contains at least one substituted amino acid AAₛ and/or w non-substituted amino acid AAₙₛ and L2 represents a (C₁-C₆)alkyl-NA₇-(C₁-C₆)alkyl group;
   A₇ is as defined above.

According to at least one embodiment, (AA)w contains at least one substituted amino acid AAₛ and L2 represents:
a (C₁-C₆)alkyl group, such as a (CH₂)₃ group;
a C(═O)—(C₁-C₆)alkyl group, such as a C(═O)—(CH₂)₃ group; or
a (C₁-C₆)alkyl-NA₇-(C₁-C₆)alkyl group, such as a (CH₂)₂—NA₇-(CH₂)₂ group in which A₇ is as defined above.

According to at least one embodiment, (AA)w contains at least one substituted amino acid AAₛ and L2 represents:
a (C₁-C₆)alkyl group, such as a (CH₂)₃ group;
a C(═O)—(C₁-C₆)alkyl group, such as a C(═O)—(CH₂)₃ group; or
a (C₁-C₆)alkyl-NA₇-(C₁-C₆)alkyl group, such as a (CH₂)₂—NA₇-(CH₂)₂ group in which A₇ is a —C(═O)—(CH₂)₂—C(═O)—NH—(CH₂)₂—SO₃H group;
   being understood that each A₇ comprising a SO₃H function can be under salt forms such as alkali metal salts, for instance sodium salts (SO₃^{- +}Na).

According to another embodiment, (AA)w contains w non-substituted amino acid AAₙₛ and L2 represents:
a (C₁-C₆)alkyl-NA₇-(C₁-C₆)alkyl group, such as a (CH₂)₂—NA₇-(CH₂)₂ group in which A₇ is as defined above.

According to another embodiment, (AA)w contains w non-substituted amino acid AAₙₛ and L2 represents:
a (C₁-C₆)alkyl-NA₇-(C₁-C₆)alkyl group, such as a (CH₂)₂—NA₇-(CH₂)₂ group in which A₇ represents:
   a C(═O)—[(CH₂)₂—O]ₐ—CH₃ group wherein “a” represents an integer ranging from 1 to 50, for instance ranging from 1 to 24, such as 4, 7 and 24, for example A₇ is a C(═O)—[(CH₂)₂—O]₄—CH₃ group, a C(═O)—[(CH₂)₂—O]₇—CH₃ group, or a C(═O)—[(CH₂)₂—O]₂₄—CH₃ group;
   a —C(═O)—(CH₂)₂—C(═O)—NH—(CH₂)₂—SO₃H group; or
   a C(═O)—(CH₂)₂—C(═O)—NH—[(CH₂)₂—O]ₐ—(CH₂)₂—C(═O)—NH—(CH₂)₂—SO₃H group wherein “a” represents an integer ranging from 1 to 50, such as ranging from 1 to 24, for example 4, such as A₇ is —C(═O)—(CH₂)₂—C(═O)—NH—[(CH₂)₂—O]₄—(CH₂)₂—C(═O)—NH—(CH₂)₂—SO₃H group;
      being understood that each A₇ comprising a SO₃H function can be under salt forms such as alkali metal salts, for instance sodium salts (SO₃^{- +}Na); According to at least one embodiment, (AA)w contains at least one substituted amino acid AAₛ and/or w non-substituted amino acid AAₙS and L2 represents:
a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ—NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ—NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ—NA₇-(C₁-C₆)alkyl group; wherein i, A₇ and A₈ are as defined above.

According to another embodiment, (AA)w contains at least one substituted amino acid AAₛ and/or w non-substituted amino acid AAₙₛ and L2 represents:
a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-C(═O)NA₇-(C₁-C₆)alkyl group, a NA₈-(C₁-C₆)alkyl-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-C(═O)NA₇-(C₁-C₆)alkyl group, or a C(═O)—NA₈-(C₁-C₆)alkyl-NA₇-(C₁-C₆)alkyl group; wherein i, A₇ and A₈ are as defined above.

According to another embodiment, (AA)w contains at least one substituted amino acid AAₛ and/or w non-substituted amino acid AAₙₛ and L2 represents:
a NA₇-(C₁-C₆)alkyl group, a (C₁-C₆)alkyl-NA₇ group, a NA₇-(CH₂CH₂O)ᵢ(C₁-C₆)alkyl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-C(═O)NA₇-(C₁-C₆)alkyl group, a NA₈-(C₁-C₆)alkyl-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-C(═O)NA₇-(C₁-C₆)alkyl group, or a C(═O)—NA₈-(C₁-C₆)alkyl-NA₇-(C₁-C₆)alkyl group; wherein i, A₇ and A₈ are as defined above.

According to another embodiment, (AA)w contains at least one substituted amino acid AAₛ and/or w non-substituted amino acid AAₙₛ and L2 represents:
a NA₇-(C₁-C₆)alkyl group, a (C₁-C₆)alkyl-NA₇ group, a (C₁-C₆)alkyl-NA₇C(═O)—(C₁-C₆)alkyl group, a (C₁-C₆)alkyl-C(═O)NA₇-(C₁-C₆)alkyl group, a (C₁-C₆)alkyl-NA₇-(C₁-C₆)alkyl group, a C(═O)—NA₇-(C₁-C₆)alkyl group, a C(═O)—(C₁-C₆)alkyl-NA₇ group, a C(═O)—(C₁-C₆)alkyl-NA₇C(═O)—(C₁-C₆)alkyl group, a C(═O)—(C₁-C₆)alkyl-C(═O)NA₇-(C₁-C₆)alkyl group, a C(═O)—(C₁-C₆)alkyl-NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-C(═O)NA₇-(C₁-C₆)alkyl group, a NA₈-(C₁-C₆)alkyl-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-C(═O)NA₇-(C₁-C₆)alkyl group, or a C(═O)—NA₈-(C₁-C₆)alkyl-NA₇-(C₁-C₆)alkyl group; wherein A₇ and A₈ are as defined above.

According to at least another embodiment, (AA)w contains at least one substituted amino acid AAₛ and/or w non-substituted amino acid AAₙₛ and L2 comprises a A₇ representing:
a C(═O)—[(CH₂)₂—O]₄—CH₃ group; a C(═O)—[(CH₂)₂—O]₇—CH₃ group; a C(═O)—[(CH₂)₂—O]₂₄—CH₃ group; a C(═O)—(CH₂)₂—C(═O)—NH—(CH₂)₂—SO₃H group; a C(═O)—(CH₂)₂—C(═O)—NH—[(CH₂)₂—O]₄—(CH₂)₂—C(═O)—NH—(CH₂)₂—SO₃H group;
   being understood that each A₇ comprising a SO₃H function can be under salt forms such as alkali metal salts, for instance sodium salts (SO₃^{- +}Na).

According to at least another embodiment, (AA)w contains at least one substituted amino acid AAₛ and/or w non-substituted amino acid AAₙₛ and L2 represents

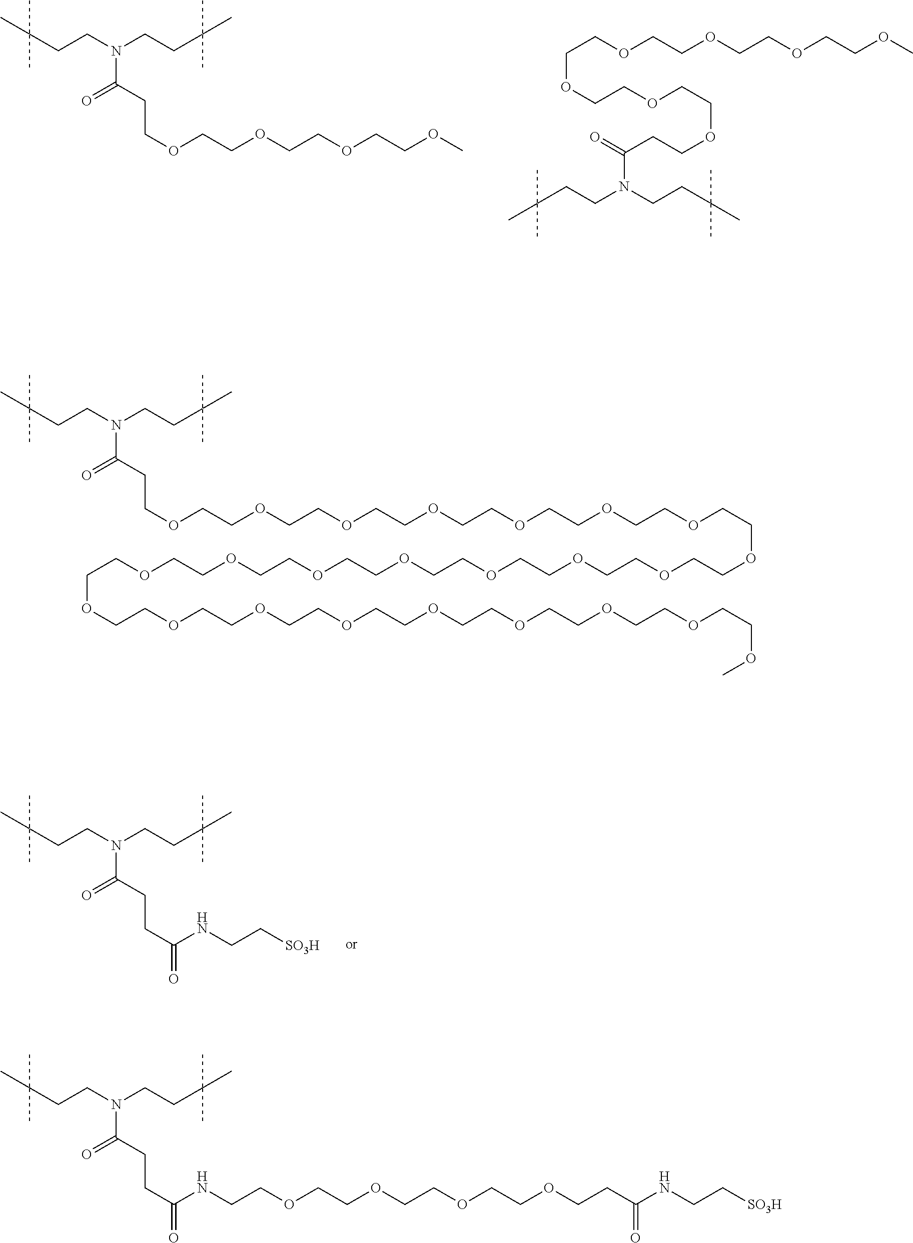

L1 may be one of the following (III1-5):

| P | Examples of L1 |
|---|---|
| H | —NA₆—ALK—NA₇— |
| | (III1) |
| | 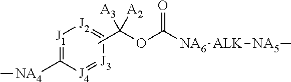 |
| | (III2) |
| OH | — |
| | (III3) |
| activated O | 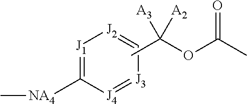 |
| | (III4) |
| | 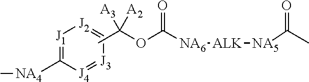 |
| | (III5) |

wherein:
J₁, J₂, J₃ and J₄ are chosen, independently of each other, from CA₁ and N;
ALK represents a (C₁-C₁₂)alkylene group, for instance (C₁-C₆)alkylene, such as of the form —(CH₂)ₙ—, n being an integer ranging from 1 to 12 and for example ranging from 1 to 6;
A₁, A₂, A₃, A₄, A₅, and A₆ represent, independently of each other, a hydrogen atom or a (C₁-C₆)alkyl group, such as a hydrogen atom or a methyl group.

AA denotes an amino acid. An amino acid is a compound of formula NH₂—CHA₁₀-COOH wherein A₁₀ represents the side chain of the AA. AA can be a substituted AAₛ or non-substituted AAₙₛ amino acid.

A non-substituted amino acid AAₙₛ denotes natural or non-natural amino acid, of configuration D or L, identical to or derived from: alanine (Ala), β-alanine, γ-aminobutyric acid, 2-amino-2-cyclohexylacetic acid, 2-amino-2-phenylacetic acid, arginine (Arg), asparagine (Asn), aspartic acid (Asp), citrulline (Cit), cysteine (Cys), α,α-dimethyl-γ-aminobutyric acid, β,β-dimethyl-γ-aminobutyric acid, glutamine (Gln), glutamic acid (Glu), glycine (Gly), homo-cysteine, selenocysteine, homo-selenocysteine, histidine (His), isoleucine (Ile), leucine (Leu), lysine (Lys), ε-acetyl-lysine (AcLys), methionine (Met), ornithine (Orn), phenylalanine (Phe), proline (Pro), serine (Ser), threonine (Thr), tryptophan (Trp), tyrosine (Tyr), and valine (Val).

For example, AAₙS represents alanine (Ala), citrulline (Cit), glycine (Gly), isoleucine (lie), leucine (Leu), lysine (Lys), ε-acetyl-lysine (AcLys), phenylalanine (Phe), tryptophan (Trp) and valine (Val).

The substituted amino acids AAₛ have the formula (VI):

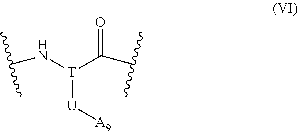

wherein:
T represents a saturated or unsaturated, linear or branched, (C₁-C₈) trivalent alkyl group, for instance T is

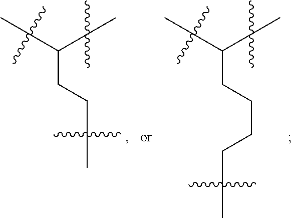 [SMILES: *CCC(*)* ; *CCCCC(*)*]

U group represents a single bond, —NHC(═O)—, —N(alkyl)C(═O)—, —C(═O)NH—, —C(═O)N(alkyl)-, —NHC(═O)NH—, —NHC(═NH)NH—, —OC(═O)—, —C(═O)O—, —OC(═O)O—, —S—, —Se—, —O—, —NH—, —N(alkyl)-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, or —O—P(═O)(OH)—O, such as U group represents —NH—C(═O)—, or —C(═O)NH—;
A₉ represents a straight or branched, saturated or unsaturated, optionally substituted C₁-C₁₆₀ hydrocarbon chain wherein optionally at least one methylene unit is independently replaced by —NH—C(═O)—, —N(alkyl)C(═O)—, —C(═O)NH—, —C(═O)N(alkyl)-, —NHC(═O)NH—, —NHC(═NH)NH—, —OC(═O)—, —C(═O)O—, —OC(═O)O—, —CH(OH)—, —CH(SO₃H)—, —CH(Oalkyl)-, —CHF—, —CF₂—, —S—, —Se—, —O—, —NH—, —N(alkyl)-, —N⁺H(alkyl)-, —N⁺(alkyl)₂-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, for instance A₉ is [(CH₂)₂—O]₄—CH₃, [(CH₂)₂—O]₂₄—CH₃,

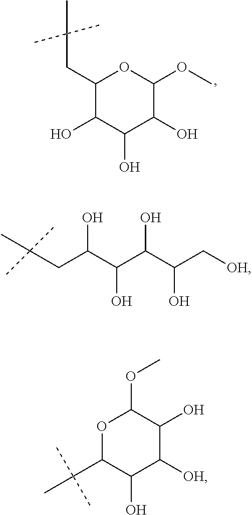

or [(CH₂)₂—O)]₄—(CH₂)₂—C(═O)—NH—(CH₂)₂—SO₃H;
being understood that each A₉ comprising a SO₃H function can be under salt forms such as alkali metal salts, for instance sodium salts (SO₃^{- +}Na).

According to a particular embodiment, the substituted amino acids AAₛ have the formula (VI):

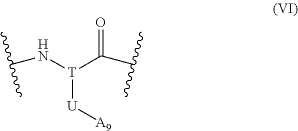

wherein:
T represents a saturated or unsaturated, linear or branched, (C₁-C₈) trivalent alkyl group, for instance T is

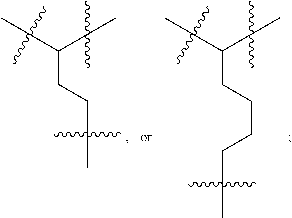 [SMILES: *CCC(*)* ; *CCCCC(*)*]

U group represents a single bond, —NHC(═O)—, —N(alkyl)C(═O)—, —C(═O)NH—, —C(═O)N(alkyl)-, —NHC(═O)NH—, —NHC(═NH)NH—, —OC(═O)—, —C(═O)O—, —OC(═O)O—, —S—, —Se—, —O—, —NH—, —N(alkyl)-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, or —O—P(═O)(OH)—O, such as U group represents —NH—C(═O)—, or —C(═O)NH—;
A₉ represents a straight or branched, saturated or unsaturated, optionally substituted C₁-C₁₆₀ hydrocarbon chain wherein optionally at least one methylene unit is independently replaced by —NH—C(═O)—, —N(alkyl)C(═O)—, —C(═O)NH—, —C(═O)N(alkyl)-, —NHC(═NH)NH—, —OC(═O)—, —C(═O)O—, —OC(═O)O—, —CH(OH)—, —CH(SO₃H)—, —CH(Oalkyl)-, —CHF—, —CF₂—, —S—, —Se—, —O—, —NH—, —N(alkyl)-, —N⁺H(alkyl)-, —N⁺(alkyl)₂-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, for instance A₉ is [(CH₂)₂—O]₄—CH₃, [(CH₂)₂—O]₂₄—CH₃,

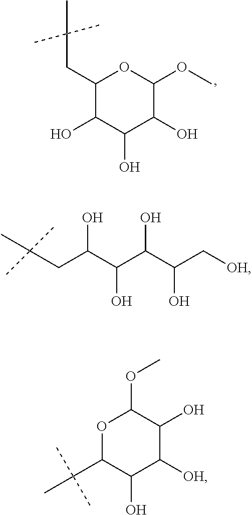

or [(CH₂)₂—O)]₄—(CH₂)₂—C(═O)—NH—(CH₂)₂—SO₃H;
being understood that each A₉ comprising a SO₃H function can be under salt forms such as alkali metal salts, for instance sodium salts (SO₃^{- +}Na).

According to another particular embodiment, the substituted amino acids AAₛ have the formula (VI):

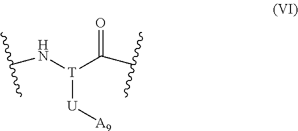

wherein:
T represents a saturated or unsaturated, linear or branched, (C₁-C₈) trivalent alkyl group, for instance T is

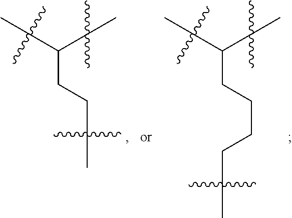 [SMILES: *CCC(*)* ; *CCCCC(*)*]

U group represents a single bond, —NHC(═O)—, —N(alkyl)C(═O)—, —C(═O)NH—, —C(═O)N(alkyl)-, —NHC(═O)NH—, —NHC(═NH)NH—, —OC(═O)—, —C(═O)O—, —OC(═O)O—, —S—, —Se—, —O—, —NH—, —N(alkyl)-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, or —O—P(═O)(OH)—O, such as U group represents —NH—C(═O)—, or —C(═O)NH—;
A₉ represents a straight or branched, saturated or unsaturated, optionally substituted C₁-C₁₆₀ hydrocarbon chain wherein optionally at least one methylene unit is independently replaced by —NH—C(═O)—, —N(alkyl)C(═O)—, —C(═O)NH—, —C(═O)N(alkyl)-, —NHC(═NH)NH—, —OC(═O)—, —C(═O)O—, —OC(═O)O—, —CH(OH)—, —CH(SO₃H)—, —CH(Oalkyl)-, —CHF—, —CF₂—, —S—, —Se—, —O—, —N(alkyl)-, —N⁺H(alkyl)-, —N⁺(alkyl)₂-, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, for instance A₉ is [(CH₂)₂—O]₄—CH₃, [(CH₂)₂—O]₂₄—CH₃,

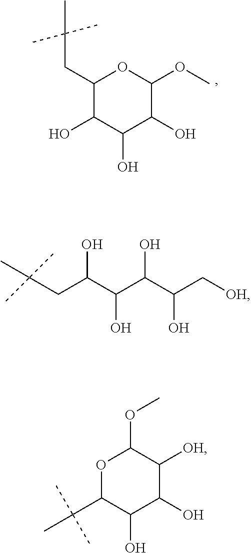

or [(CH₂)₂—O)]₄—(CH₂)₂—C(═O)—NH—(CH₂)₂—SO₃H;
being understood that each A₉ comprising a SO₃H function can be under salt forms such as alkali metal salts, for instance sodium salts (SO₃^{- +}Na).

According to another particular embodiment, the substituted amino acids AAₛ have the formula (VI):

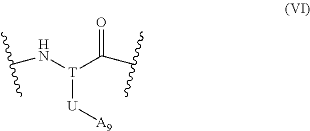

wherein:
T represents a saturated or unsaturated, linear or branched, (C₁-C₈) trivalent alkyl group, for instance T is

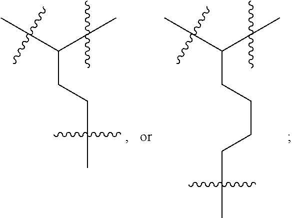 [SMILES: *CCC(*)* ; *CCCCC(*)*]

U group represents a single bond, —NHC(═O)—, —N(alkyl)C(═O)—, —C(═O)NH—, —C(═O)N(alkyl)-, —NHC(═O)NH—, —NHC(═NH)NH—, —OC(═O)—, —C(═O)O—, —OC(═O)O—, —S—, —Se—, —O—, —NH—, —N(alkyl)-, —C(═O)—, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, or —O—P(═O)(OH)—O, such as U group represents —NH—C(═O)—, or —C(═O)NH—;
A₉ represents a straight or branched, saturated or unsaturated, optionally substituted C₁-C₁₆₀ hydrocarbon chain wherein optionally at least one methylene unit is independently replaced by —NH—C(═O)—, —N(alkyl)C(═O)—, —C(═O)NH—, —C(═O)N(alkyl)-, —C(═O)O—, —OC(═O)O—, —CH(OH)—, —CH(SO₃H)—, —CH(Oalkyl)-, —CHF—, —CF₂—, —Se—, —O—, —N(alkyl)-, —N⁺H(alkyl)-, —N⁺(alkyl)₂-, —OP(═O)—, —S(═O)—, —S(═O)₂—, —NHS(═O)₂—, —N(alkyl)S(═O)₂—, —S(═O)₂NH—, —S(═O)₂N(alkyl)-, —P(═O)(OH)—, —P(═O)(OH)O—, —O—P(═O)(OH)—, —O—P(═O)(OH)—O— or a heterocycloalkyl group optionally substituted with at least one substituent, identical or different, chosen from —OH, —Oalkyl, -alkyl, a halogen atom, —NH₂, —NHalkyl, and —N(alkyl)₂, for instance A₉ is [(CH₂)₂—O]₄—CH₃,

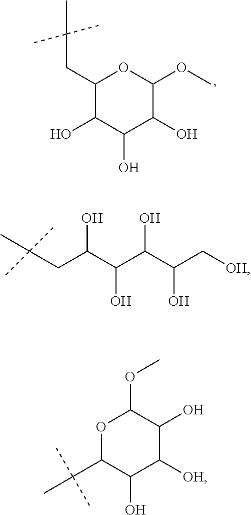

or [(CH₂)₂—O)]₄—(CH₂)₂—C(═O)—NH—(CH₂)₂—SO₃H;
being understood that each A₉ comprising a SO₃H function can be under salt forms such as alkali metal salts, for instance sodium salts (SO₃^{- +}Na).

Examples of T that may be mentioned include:

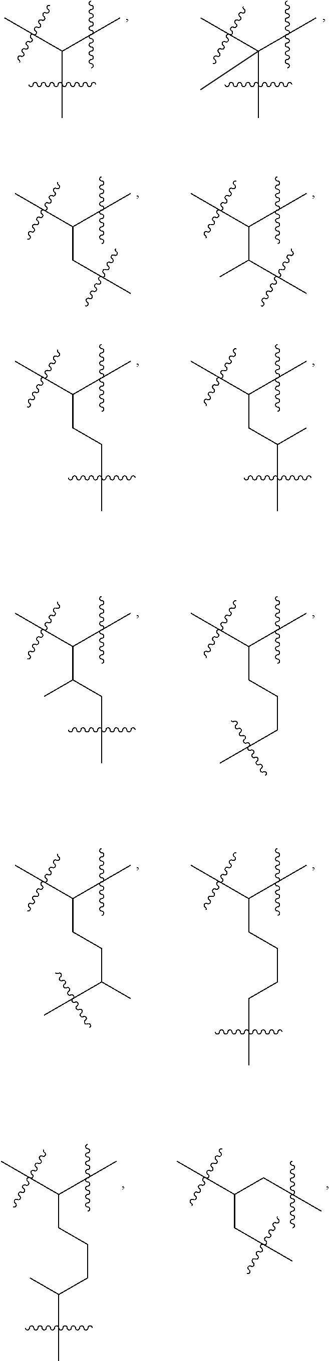

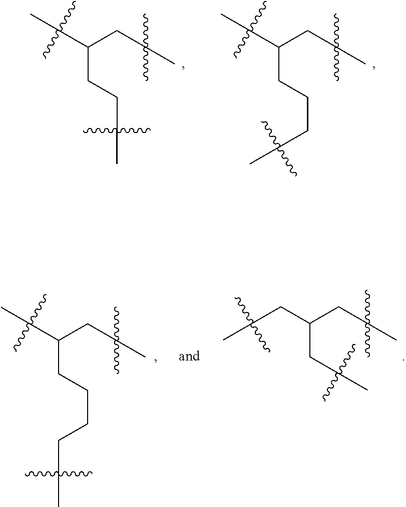

According at least one embodiment, the substituted amino acids AAₛ have the formula (VI) wherein:
T represents a saturated or unsaturated, linear or branched, (C₁-C₈) trivalent alkyl group, such as

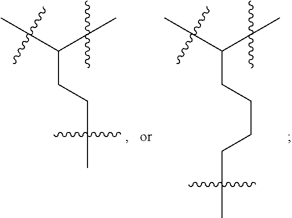 [SMILES: *CCC(*)* ; *CCCCC(*)*]

U group represents —NH—C(═O)—, or —C(═O)NH—;
A₉ represents a straight or branched, saturated or unsaturated, optionally substituted C₁-C₁₆₀ hydrocarbon chain wherein optionally at least one methylene unit is independently replaced by —C(═O)NH—, —CH(OH)—, —CH(SO₃H)—, —O—, —NH—, or —C(═O)—, or a heterocycloalkyl group optionally substituted with at least one substituent, identical or different, chosen from —OH, —Oalkyl, -alkyl,
   such as A₉ is [(CH₂)₂—O]₄—CH₃, [(CH₂)₂—O)]₂₄—CH₃,

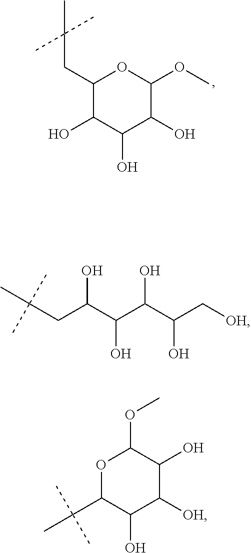

or [(CH₂)₂—O]₄—(CH₂)₂—C(═O)—NH—(CH₂)₂—SO₃H;
being understood that each A₉ comprising a SO₃H function can be under salt forms such as alkali metal salts, for instance sodium salts (SO₃^{- +}Na).

According to another embodiment, the substituted amino acids AAₛ have the formula (VI) wherein:
T represents

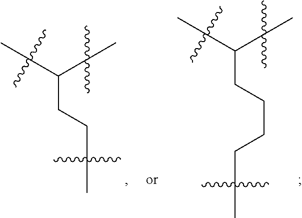 [SMILES: *CCC(*)* ; *CCCCC(*)*]

U group represents —NH—C(═O)—, or —C(═O)NH—;
A₉ represents [(CH₂)₂—O]₄—CH₃, [(CH₂)₂—O]₂₄—CH₃,

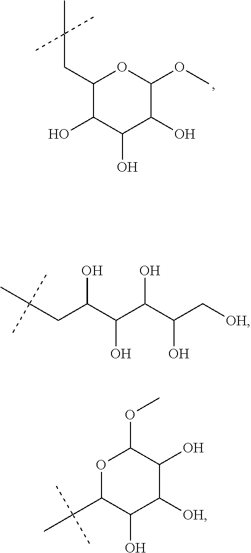

or [(CH₂)₂—O]₄—(CH₂)₂—C(═O)—NH—(CH₂)₂—SO₃H;
being understood that each A₉ comprising a SO₃H function can be under salt forms such as alkali metal salts, for instance sodium salts (SO₃^{- +}Na).

According to a yet another embodiment, the substituted amino acids AAₛ have the formula (VI) wherein:
T represents

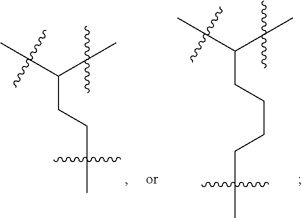 [SMILES: *CCC(*)* ; *CCCCC(*)*]

U group represents —NH—C(═O)—, or —C(═O)NH—;
A₉ represents —[(CH₂)₂—O]_{b}—CH₃ wherein “b” represents an integer ranging from 1 to 50, for instance ranging from 1 to 24, such as 4, 7 and 24, for example 4 or 24.

The sequence (AA)_{w} has the formula:

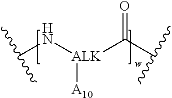

in which A₁₀ represents the side chain of one of the substituted AAₛ or non-substituted AAₙₛ amino acids described above.

Examples of sequences of non-substituted amino acids (AAₙₛ)w are as follows: Gly-Gly, Phe-Lys, Val-Lys, Val-AcLys, Val-Cit, Phe-Phe-Lys, D-Phe-Phe-Lys, Gly-Phe-Lys, Ala-Lys, Val-Ala, Phe-Cit, Phe-Gly, Leu-Cit, Ile-Cit, Trp-Cit, Phe-Ala, Ala-Phe, Gly-Gly-Gly, Gly-Ala-Phe, Gly-Phe-Gly, Gly-Val-Cit, Gly-Phe-Leu-Cit (SEQ ID No. 3), Gly-Phe-Leu-Gly (SEQ ID No. 4), Ala-Leu-Ala-Leu (SEQ ID No. 5).

According to at least one embodiment, the sequence (AAₙₛ)w of non-substituted amino acids AAₙₛ is selected from the following list: Gly-Gly, Phe-Lys, Val-Lys, Val-AcLys, Val-Cit, Phe-Phe-Lys, D-Phe-Phe-Lys, Gly-Phe-Lys, Ala-Lys, Val-Ala, Phe-Cit, Phe-Gly, Leu-Cit, Ile-Cit, Trp-Cit, Phe-Ala, Ala-Phe, Gly-Gly-Gly, Gly-Ala-Phe, Gly-Phe-Gly, Gly-Val-Cit, Gly-Phe-Leu-Cit (SEQ ID No. 3), Gly-Phe-Leu-Gly (SEQ ID No. 4), and Ala-Leu-Ala-Leu (SEQ ID No. 5), such as Val-Ala and Val-Cit, for instance Val-Ala.

According to at least another embodiment, the sequence (AAₛ)w containing at least one substituted amino acid AAₛ is selected from the list:

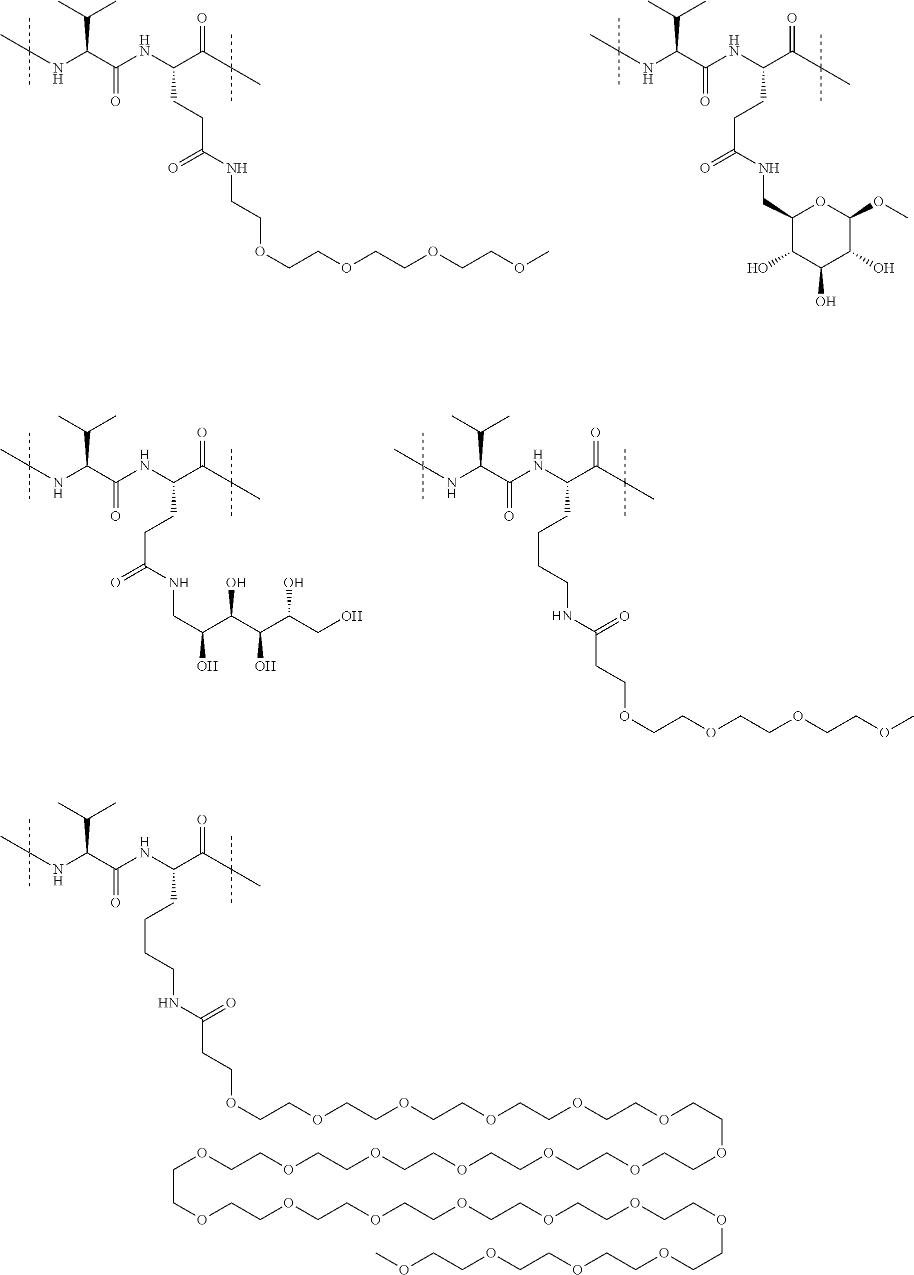

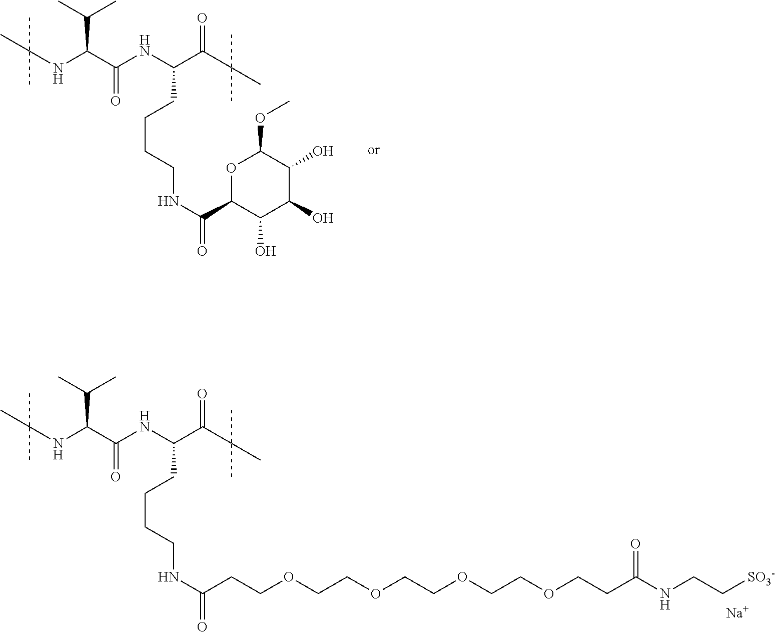

Among the linkers L of formula (II) that are the subject matter of the disclosure, mention may be made for example of the following compounds:

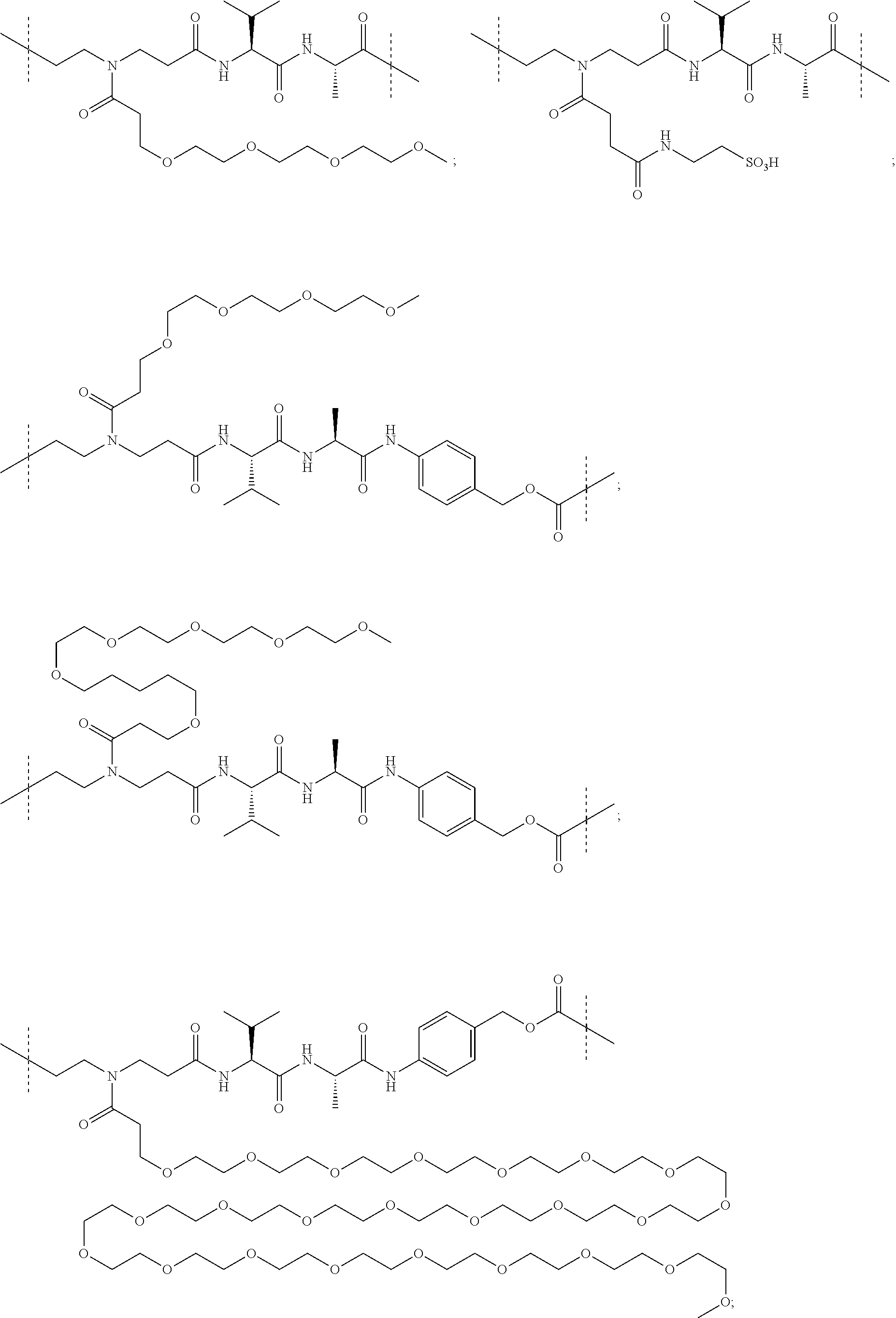

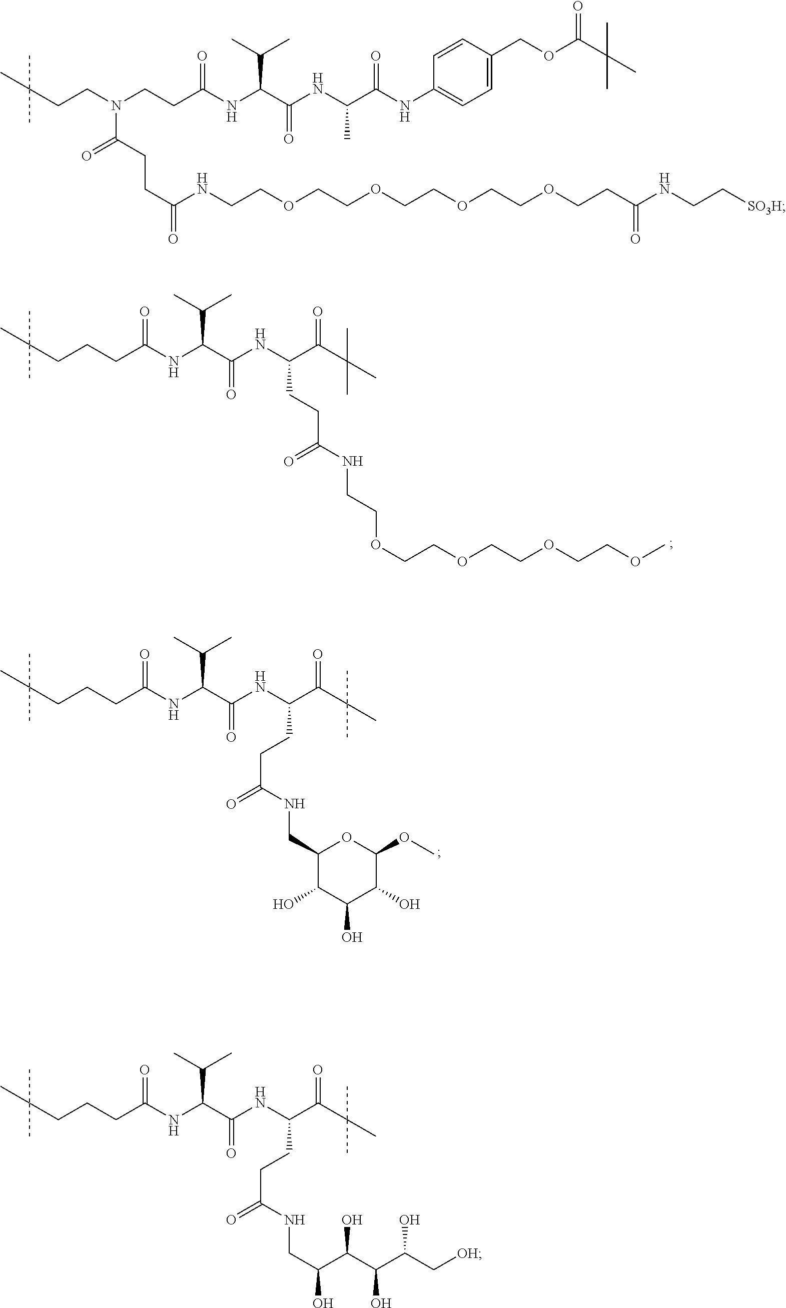

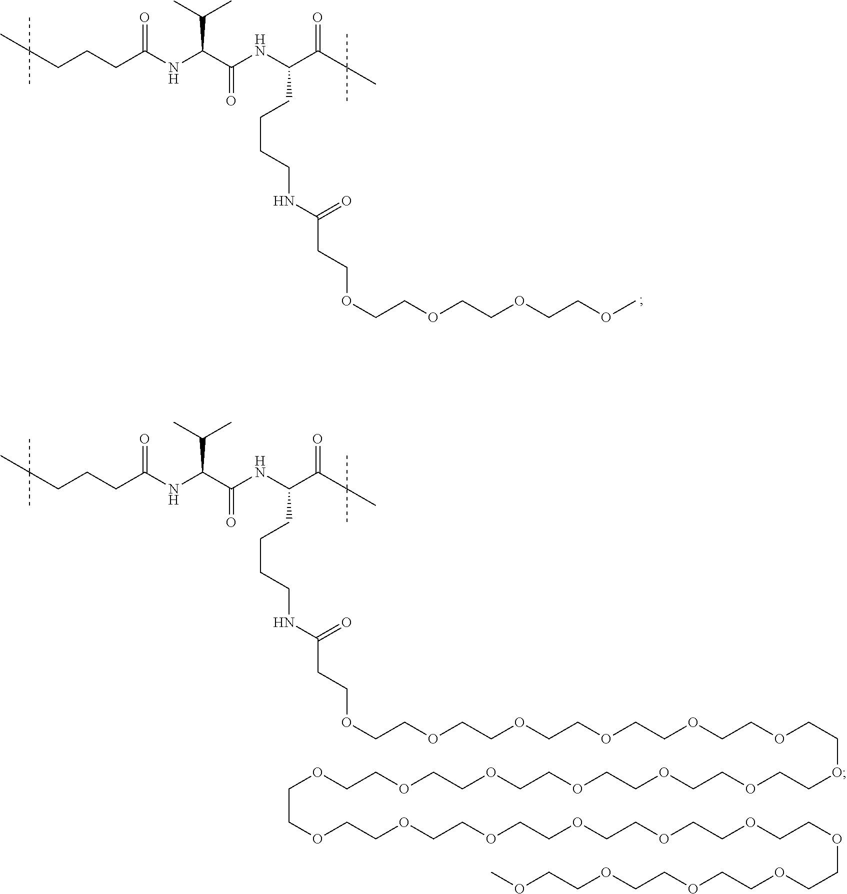

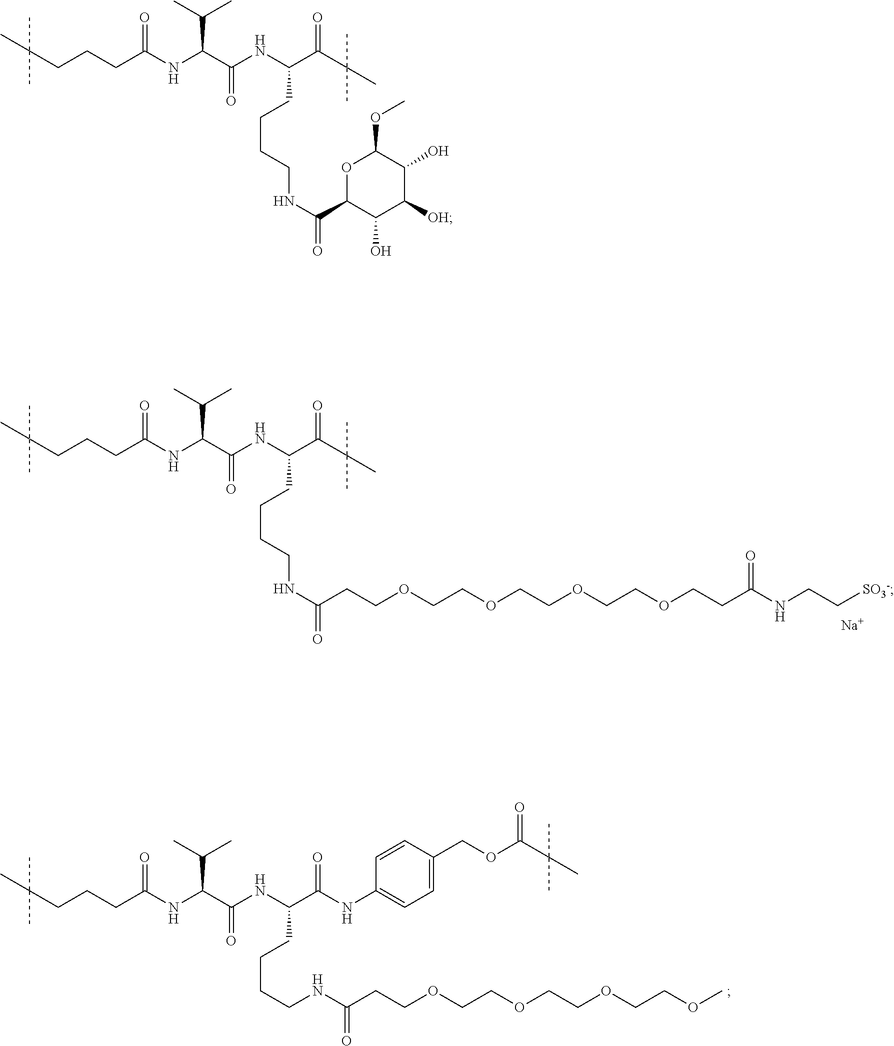

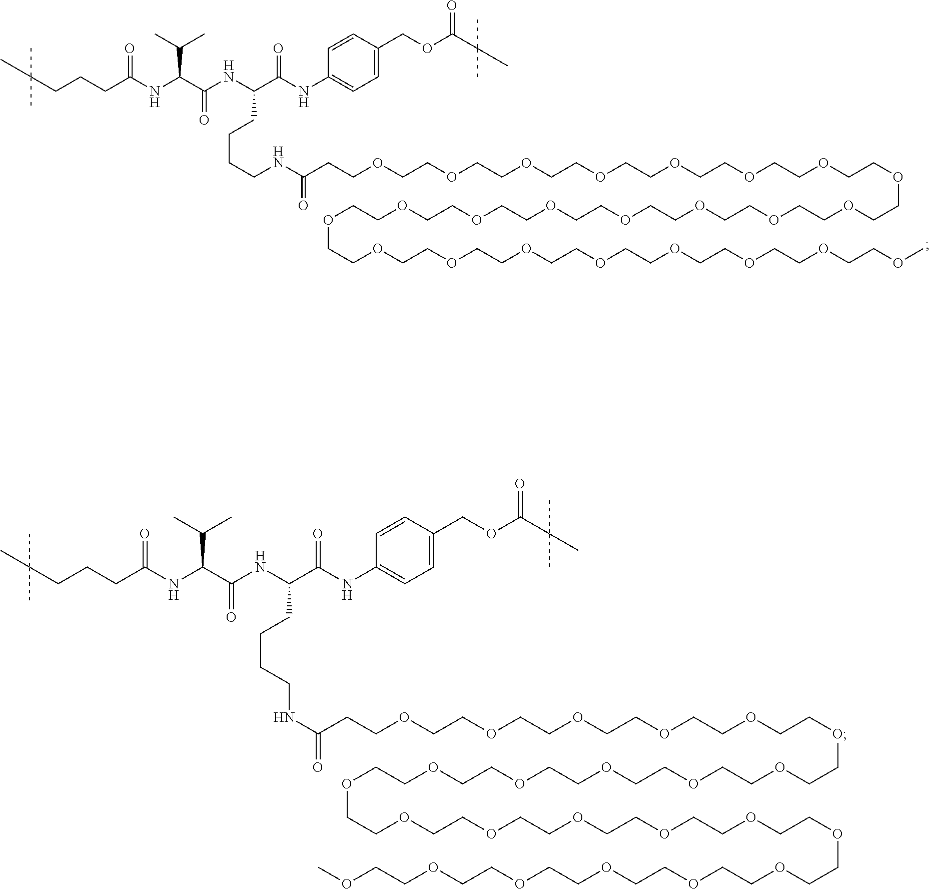

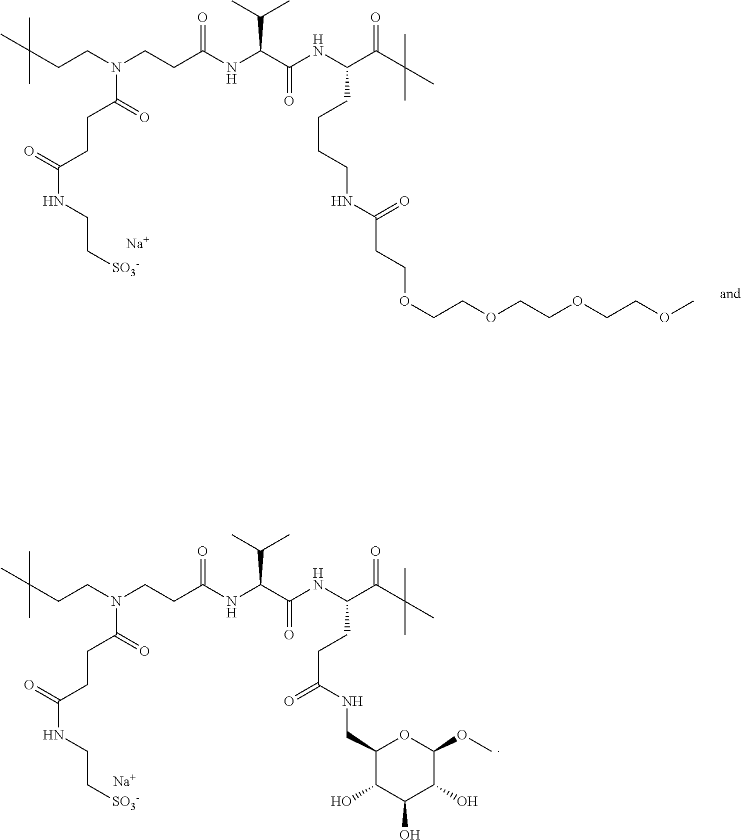

Examples of RCG1 that may be mentioned include:
(i) a RₐZₐ—C(═O)— reactive group for which:
Zₐ represents a single bond, —O— or —NH, such as —O—, and
Rₐ represents a hydrogen atom, a (C₁-C₆)alkyl, a (C₃-C₇)cycloalkyl, an alkenyl, an aryl, a heteroaryl or a (C₃-C₇)heterocycloalkyl group. The aryl group, the heteroaryl group and/or the (C₃-C₇)heterocycloalkyl group may be substituted by 1 to 5 atoms/groups chosen from a halogen atom, such as a fluorine atom, an alkyl group, an alkoxy group, a hydroxyl group, an oxo group, a nitro group and a cyano group;
(ii) one of the following reactive groups: a maleimido

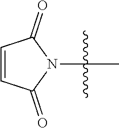 [SMILES: *N1C(=O)C=CC1=O]

group; a haloacetamido

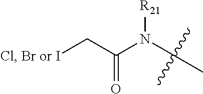

group with R₂₁ representing a hydrogen atom or a (C₁-C₆)alkyl group, such as Me; Cl—; N₃—; HO—; HS—; an activated disulfide such as

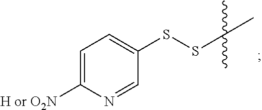

H₂N—; HC≡C— or an activated C≡C such as a cyclooctyne moiety for instance a DBCO-amine

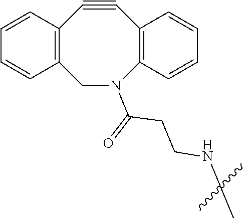 [SMILES: *NCCC(=O)N1Cc2ccccc2C#Cc2ccccc21]

### or BCN

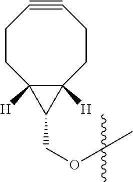 [SMILES: *OC[C@@H]1[C@@H]2CCC#CCC[C@@H]21]

### or MFCO

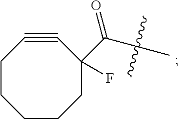 [SMILES: *C(=O)C1(F)C#CCCCCC1]

an O-alkyl hydroxylamine or a Pictet-Spengler reaction substrate such as

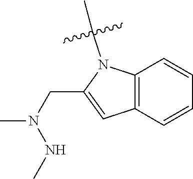 [SMILES: *n1c(CN(C)NC)cc2ccccc21]

described in Agarwal P., et al., Bioconjugate Chem 2013, 24, 846-851.

According to a particular embodiment, RCG1 may be:
(i) a RₐZₐ—C(═O)— reactive group for which:
Zₐ represents a single bond, —O— or —NH, such as —O—, and
Rₐ represents a hydrogen atom, a (C₁-C₆)alkyl, a (C₃-C₇)cycloalkyl, an alkenyl, an aryl, a heteroaryl or a (C₃-C₇)heterocycloalkyl group. The aryl group, the heteroaryl group and/or the (C₃-C₇)heterocycloalkyl group may be substituted by 1 to 5 atoms/groups chosen from a halogen atom, such as a fluorine atom, an alkyl group, an alkoxy group, a hydroxyl group, an oxo group, a nitro group and a cyano group;
(ii) one of the following reactive groups: a haloacetamido

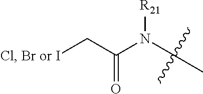

group with R₂₁ representing a hydrogen atom or a (C₁-C₆)alkyl group, such as Me; Cl—; N₃—; HO—; HS—; an activated disulfide such as

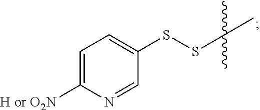

H₂N—; HC≡C— or an activated C≡C such as a cyclooctyne moiety for instance a DBCO-amine

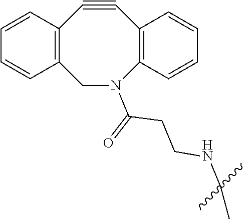 [SMILES: *NCCC(=O)N1Cc2ccccc2C#Cc2ccccc21]

### or BCN

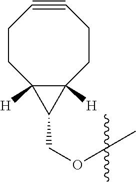 [SMILES: *OC[C@@H]1[C@@H]2CCC#CCC[C@@H]21]

### or MFCO

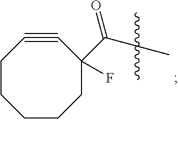

an O-alkyl hydroxylamine or a Pictet-Spengler reaction substrate such as

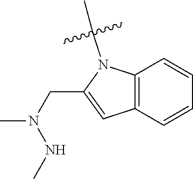 [SMILES: *n1c(CN(C)NC)cc2ccccc21]

described in Agarwal P., et al., Bioconjugate Chem 2013, 24, 846-851.

For instance, RₐZₐ— may represent HO—, CH₃O—, CH₂=CH—CH₂θ—,

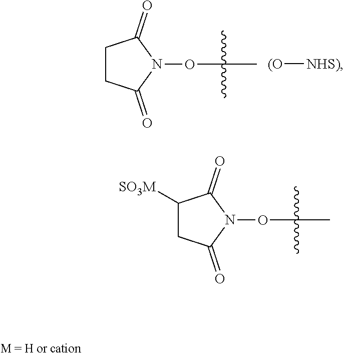

where cation represents for example sodium, potassium or cesium or,

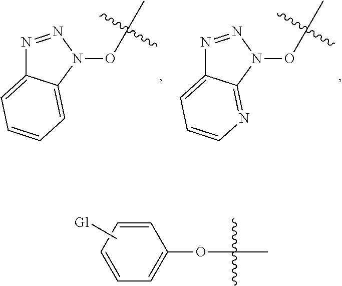

group in which
GI represents at least one electroinductive group such as —NO₂ or a halogen atom, such as a fluorine atom (F). They may be, for example, the following groups:

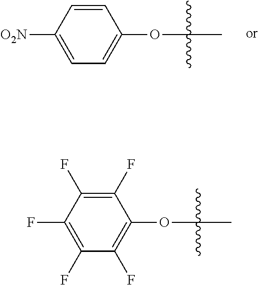 [SMILES: *Oc1ccc([N+](=O)[O-])cc1 ; *Oc1c(F)c(F)c(F)c(F)c1F]

Another type of RₐZₐ—C(═O)— group is the following:

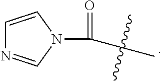 [SMILES: *C(=O)n1ccnc1]

For instance, RCG1 may be chosen from one of those described in the examples that is to say chosen from the following groups: COOH,

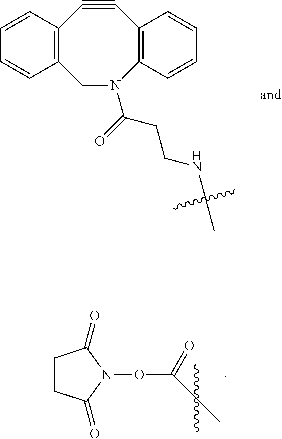 [SMILES: *NCCC(=O)N1Cc2ccccc2C#Cc2ccccc21 ; *C(=O)ON1C(=O)CCC1=O]

The present disclosure further relates to cryptophycin payloads of formula (IV):

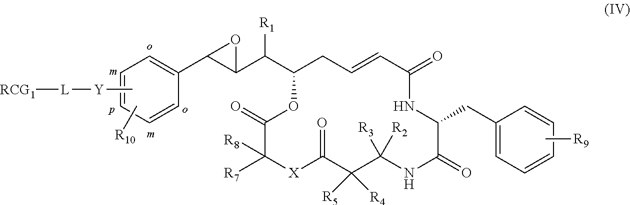

wherein:
R₁ represents a (C₁-C₆)alkyl group,
R₂ and R₃ represent, independently of each other, a hydrogen atom or a (C₁-C₆)alkyl group;
or alternatively R₂ and R₃ form together with the carbon atom to which they are attached a (C₃-C₆)cycloalkyl or a (C₃-C₆)heterocycloalkyl group;
R₄ and R₅ represent, independently of each other, a hydrogen atom or a (C₁-C₆)alkyl group or a (C₁-C₆)alkyl-NH(R₁₂) group or a (C₁-C₆)alkyl-OH group or a (C₁-C₆)alkyl-SH group or a (C₁-C₆)alkyl-C(═O)₂H group;
or alternatively R₄ and R₅ form together with the carbon atom to which they are attached a (C₃-C₆)cycloalkyl or a (C₃-C₆)heterocycloalkyl group;
X represents O or N(R₆);
R₆ represents a hydrogen atom or a (C₁-C₆)alkyl group;
R₇ and R₈ represent, independently of each other, a hydrogen atom or a (C₁-C₆)alkyl group or a (C₁-C₆)alkyl-C(═O)₂H group or a (C₁-C₆)alkyl-N(C₁-C₆)alkyl₂ group;
or alternatively R₇ and R₈ form together with the carbon atom to which they are attached a (C₃-C₆)cycloalkyl group or a (C₃-C₆)heterocycloalkyl group;
R₉ represents at least one substituent of the phenyl nucleus chosen, independently of each other, from: a hydrogen atom, —OH, (C₁-C₄)alkoxy, a halogen atom, —NH₂, —NH(C₁-C₆)alkyl or —N(C₁-C₆)alkyl₂ or —NH(C₁-C₆)cycloalkyl or (C₃-C₆)heterocycloalkyl group;
R₁₀ represents at least one substituent of the phenyl nucleus chosen from a hydrogen atom and a (C₁-C₄)alkyl group;
Y represents
   —NR₁₁—(C₁-C₆)alkyl-, such as —NR₁₁—(CH₂)ₙ— like

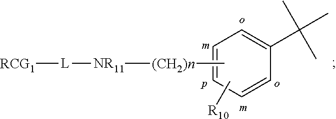

—O—(C₁-C₆)alkyl-, such as —O—(CH₂)ₙ— like

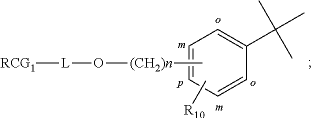

—S—(C₁-C₆)alkyl-, such as —S—(CH₂)ₙ— like

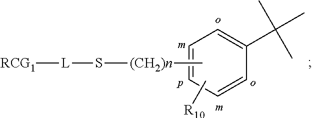

Y being positioned in an ortho (o), meta (m) or para (p) position of the phenyl nucleus;
R₁₁ and R₁₂ represent, independently of each other, a hydrogen atom or (C₁-C₆)alkyl, for instance a hydrogen atom or a methyl group;
n represents an integer ranging from 1 to 6;
L is defined as in formula (I) and represents a linker of formula (II) as defined in the present disclosure;
RCG1 represents a reactive chemical group present at the end of the linker L, RCG1 being reactive towards a chemical group present on a polypeptide such as an antibody. RCG1 is defined above.

Each substituent R₁ to R₁₂ may also adopt one of the spatial configurations (e.g. R or S or alternatively Z or E) as described in the examples. The compounds of formula (IV) may contain at least one asymetric carbon atom. They may therefore exist in the form of enantiomers or diastereoisomers. These enantiomers and diastereoisomers, and also mixtures thereof, including racemic mixtures, form part of the disclosure.

As an example, the cryptophycin compound of formula (IV) may be one of the cryptophycin compounds of formula (I) described in WO2011/001052 (such as one of D₁-D₈) or in PCT/EP2016/076603, (such as one of D₁-D₁₉), as mentioned above.

Or the cryptophycin compound may be an equivalent unit described in one of the examples.

Among the compounds of formula (IV) that are subject matter of the disclosure, a group of compounds is composed of the compounds for which R₁ represents a (C₁-C₆)alkyl, such as a methyl group.

Among the compounds of formula (IV) that are subject matter of the disclosure, a group of compounds is composed of the compounds for which each of R₂ and R₃ represents a hydrogen atom.

Among the compounds of formula (IV) that are subject matter of the disclosure, a group of compounds is composed of the compounds for which one of R₂ and R₃ represents a (C₁-C₆)alkyl, such as a methyl group, and the other one represents a hydrogen atom.

Among the compounds of formula (IV) that are subject matter of the disclosure, a group of compounds is composed of the compounds for which R₂ and R₃ form together with the carbon atom to which they are attached a (C₃-C₆)cycloalkyl group, such as a cyclopropyl group.

Among the compounds of formula (IV) that are subject matter of the disclosure, a group of compounds is composed of the compounds for which each of R₄ and R₅ represents a (C₁-C₆)alkyl, such as a methyl group.

Among the compounds of formula (IV) that are subject matter of the disclosure, a group of compounds is composed of the compounds for which X represents an oxygen atom, that is to say O.

Among the compounds of formula (IV) that are subject matter of the disclosure, a group of compounds is composed of the compounds for which X represents NH.

Among the compounds of formula (IV) that are subject matter of the disclosure, a group of compounds is composed of the compounds for which R₇ and R₈ represent independently of each other an hydrogen atom or a (C₁-C₆)alkyl group, such as an isobutyl group or a neopentyl group.

Among the compounds of formula (IV) that are subject matter of the disclosure, a group of compounds is composed of the compounds for which R₉ represents two substituents selected from a methoxy group and a chlorine atom, such as the two R₉ substituents are 3-Cl and 4-methoxy. For instance, the phenyl nucleus comprises two substituents in positions 3 and 4 on the phenyl nucleus.

Among the compounds of formula (IV) that are subject matter of the disclosure, a group of compounds is composed of the compounds for which R₁₀ represents a hydrogen atom.

Among the compounds of formula (IV) that are subject matter of the disclosure, a group of compounds is composed of the compounds for which Y is positioned in the para position of the phenyl nucleus.

Among the compounds of formula (IV) that are subject matter of the disclosure, a group of compounds is composed of the compounds for which Y represents NR₁₁—(C₁-C₆)alkyl, such as NR₁₁—(C₁-C₃)alkyl, for instance NH—CH₂.

Among the compounds of formula (IV) that are subject matter of the disclosure, a group of compounds is composed of the compounds for which L of formula (II) comprises at least one substituted amino acid AAₛ in the sequence of w amino acids (AA)w, L1 and L2 are as defined in formula (II).

Among the compounds of formula (IV) that are subject matter of the disclosure, a group of compounds is composed of the compounds for which L of formula (II) comprises a sequence of w non-substituted amino-acid AAₙₛ, L1 and L2 are as defined in formula (II).

Among the compounds of formula (IV) that are subject matter of the disclosure, a group of compounds is composed of the compounds for which (AA)w in L of formula (II) contains at least one substituted amino acid AAₛ and L2 represents:
a (C₁-C₆)alkyl group, such as a (CH₂)₃ group;
a C(═O)—(C₁-C₆)alkyl group, such as a C(═O)—(CH₂)₃ group; or
a (C₁-C₆)alkyl-NA₇-(C₁-C₆)alkyl group, such as a (CH₂)₂—NA₇-(CH₂)₂ group in which A₇ is as defined above.

Among the compounds of formula (IV) that are subject matter of the disclosure, a group of compounds is composed of the compounds for which (AA)w in L of formula (II) contains at least one substituted amino acid AAₛ and L2 represents:
a (C₁-C₆)alkyl group, such as a (CH₂)₃ group;
a C(═O)—(C₁-C₆)alkyl group, such as a C(═O)—(CH₂)₃ group; or
a (C₁-C₆)alkyl-NA₇-(C₁-C₆)alkyl group, such as a (CH₂)₂—NA₇-(CH₂)₂ group in which A₇ is a —C(═O)—(CH₂)₂—C(═O)—NH—(CH₂)₂—SO₃H group;
   being understood that each A₇ comprising a SO₃H function can be under salt forms such as alkali metal salts, for instance sodium salts (SO₃^{- +}Na).

Among the compounds of formula (IV) that are subject matter of the disclosure, a group of compounds is composed of the compounds for which (AA)w in L of formula (II) contains w non-substituted amino acid AAₙs and L2 represents:
a (C₁-C₆)alkyl-NA₇-(C₁-C₆)alkyl group, such as a (CH₂)₂—NA₇-(CH₂)₂ group in which A₇ is as defined above.

Among the compounds of formula (IV) that are subject matter of the disclosure, a group of compounds is composed of the compounds for which (AA)w in L of formula (II) contains w non-substituted amino acid AAₙS and L2 represents:
a (C₁-C₆)alkyl-NA₇-(C₁-C₆)alkyl group, such as a (CH₂)₂—NA₇-(CH₂)₂ group in which A₇ represents:
   a C(═O)—[(CH₂)₂—O]ₐ—CH₃ group wherein “a” represents an integer ranging from 1 to 50, for instance ranging from 1 to 24, such as 4, 7 and 24, for example A₇ is a C(═O)—[(CH₂)₂—O]₄—CH₃ group, a C(═O)—[(CH₂)₂—O]₇—CH₃ group, or a C(═O)—[(CH₂)₂—O]₂₄—CH₃ group;
   a —C(═O)—(CH₂)₂—C(═O)—NH—(CH₂)₂—SO₃H group; or
   a C(═O)—(CH₂)₂—C(═O)—NH—[(CH₂)₂—O]ₐ—(CH₂)₂—C(═O)—NH—(CH₂)₂—SO₃H group wherein “a” represents an integer ranging from 1 to 50, such as ranging from 1 to 24, for example 4, such as A₇ is —C(═O)—(CH₂)₂—C(═O)—NH—[(CH₂)₂—O]₄—(CH₂)₂—C(═O)—NH—(CH₂)₂—SO₃H group;
      being understood that each A₇ comprising a SO₃H function can be under salt forms such as alkali metal salts, for instance sodium salts (SO₃^{- +}Na);

Among the compounds of formula (IV) that are subject matter of the disclosure, a group of compounds is composed of the compounds for which (AA)w in L of formula (II) contains at least one substituted amino acid AAₛ and/or w non-substituted amino acid AAₙₛ and L2 represents:
a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ—NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ—NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ—NA₇-(C₁-C₆)alkyl group; wherein i, A₇ and A₈ are as defined above.

Among the compounds of formula (IV) that are subject matter of the disclosure, a preferred group of compounds is composed of the compounds for which (AA)w in L of formula (II) contains at least one substituted amino acid AAₛ and/or w non-substituted amino acid AAₙS and L2 represents:
a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-C(═O)NA₇-(C₁-C₆)alkyl group, a NA₈-(C₁-C₆)alkyl-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-C(═O)NA₇-(C₁-C₆)alkyl group, or a C(═O)—NA₈-(C₁-C₆)alkyl-NA₇-(C₁-C₆)alkyl group; wherein i, A₇ and A₈ are as defined above.

Among the compounds of formula (IV) that are subject matter of the disclosure, a more preferred group of compounds is composed of the compounds for which (AA)w in L of formula (II) contains at least one substituted amino acid AAₛ and/or w non-substituted amino acid AAₙs and L2 represents:
a NA₇-(C₁-C₆)alkyl group, a (C₁-C₆)alkyl-NA₇ group, a NA₇-(CH₂CH₂O)ᵢ(C₁-C₆)alkyl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-C(═O)NA₇-(C₁-C₆)alkyl group, a NA₈-(C₁-C₆)alkyl-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-C(═O)NA₇-(C₁-C₆)alkyl group, or a C(═O)—NA₈-(C₁-C₆)alkyl-NA₇-(C₁-C₆)alkyl group; wherein i, A₇ and A₈ are as defined above.

Among the compounds of formula (IV) that are subject matter of the disclosure, a still more preferred group of compounds is composed of the compounds for which (AA)w in L of formula (II) contains at least one substituted amino acid AAₛ and/or w non-substituted amino acid AAₙₛ and L2 represents:
a NA₇-(C₁-C₆)alkyl group, a (C₁-C₆)alkyl-NA₇ group, a (C₁-C₆)alkyl-NA₇C(═O)—(C₁-C₆)alkyl group, a (C₁-C₆)alkyl-C(═O)NA₇-(C₁-C₆)alkyl group, a (C₁-C₆)alkyl-NA₇-(C₁-C₆)alkyl group, a C(═O)—NA₇-(C₁-C₆)alkyl group, a C(═O)—(C₁-C₆)alkyl-NA₇ group, a C(═O)—(C₁-C₆)alkyl-NA₇C(═O)—(C₁-C₆)alkyl group, a C(═O)—(C₁-C₆)alkyl-C(═O)NA₇-(C₁-C₆)alkyl group, a C(═O)—(C₁-C₆)alkyl-NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-C(═O)NA₇-(C₁-C₆)alkyl group, a NA₈-(C₁-C₆)alkyl-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-C(═O)NA₇-(C₁-C₆)alkyl group, or a C(═O)—NA₈-(C₁-C₆)alkyl-NA₇-(C₁-C₆)alkyl group; wherein A₇ and A₈ are as defined above.

Among the compounds of formula (IV) that are subject matter of the disclosure, a most preferred group of compounds is composed of the compounds for which (AA)w in L of formula (II) contains at least one substituted amino acid AAₛ and/or w non-substituted amino acid AAₙs and L2 represents:
a (C₁-C₆)alkyl-NA₇-(C₁-C₆)alkyl group; wherein A₇ is as defined above.

Among the compounds of formula (IV) that are subject matter of the disclosure, a group of compounds is composed of the compounds for which (AA)w in L of formula (II) contains at least one substituted amino acid AAₛ and/or w non-substituted amino acid AAₙₛ and L2 comprises a A₇ representing:
a C(═O)—[(CH₂)₂—O]₄—CH₃ group; a C(═O)—[(CH₂)₂—O]₇—CH₃ group; a C(═O)—[(CH₂)₂—O]₂₄—CH₃ group; a C(═O)—(CH₂)₂—C(═O)—NH—(CH₂)₂—SO₃H group; a C(═O)—(CH₂)₂—C(═O)—NH—[(CH₂)₂—O]₄—(CH₂)₂—C(═O)—NH—(CH₂)₂—SO₃H group;
being understood that each A₇ comprising a SO₃H function can be under salt forms such as alkali metal salts, for instance sodium salts (SO₃^{- +}Na).

Among the compounds of formula (IV) that are subject matter of the disclosure, a group of compounds is composed of the compounds for which (AA)w in L of formula (II) contains at least one substituted amino acid AAₛ and/or w non-substituted amino acid AAₙₛ and L2 represents:

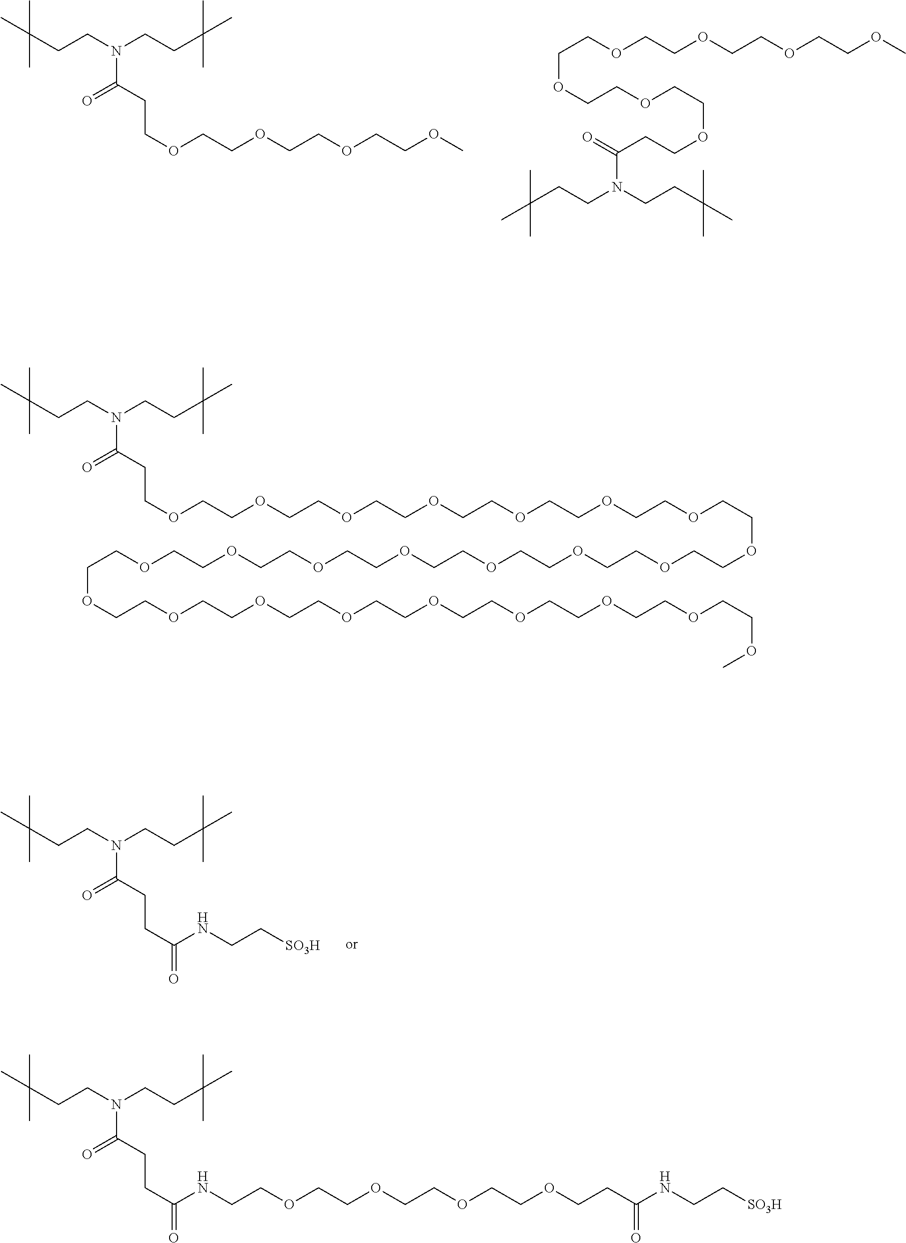 [SMILES: COCCOCCOCCOCCC(=O)N(CCC(C)(C)C)CCC(C)(C)C ; COCCOCCOCCOCCOCCOCCOCCC(=O)N(CCC(C)(C)C)CCC(C)(C)C ; COCCOCCOCCOCCOCCOCCOCCOCCOCCOCCOCCOCCOCCOCCOCCOCCOCCOCCOCCOCCOCCOCCOCCOCCC(=O)N(CCC(C)(C)C)CCC(C)(C)C ; CC(C)(C)CCN(CCC(C)(C)C)C(=O)CCC(=O)NCCS(=O)(=O)O ; "or" ; CC(C)(C)CCN(CCC(C)(C)C)C(=O)CCC(=O)NCCOCCOCCOCCOCCC(=O)NCCS(=O)(=O)O]

Among the compounds of formula (IV) that are subject matter of the disclosure, a group of compounds is composed of the compounds comprising in L a sequence (AA_{S})w containing at least one substituted amino acid AA_{S}, (AA_{S})w being selected from the list:

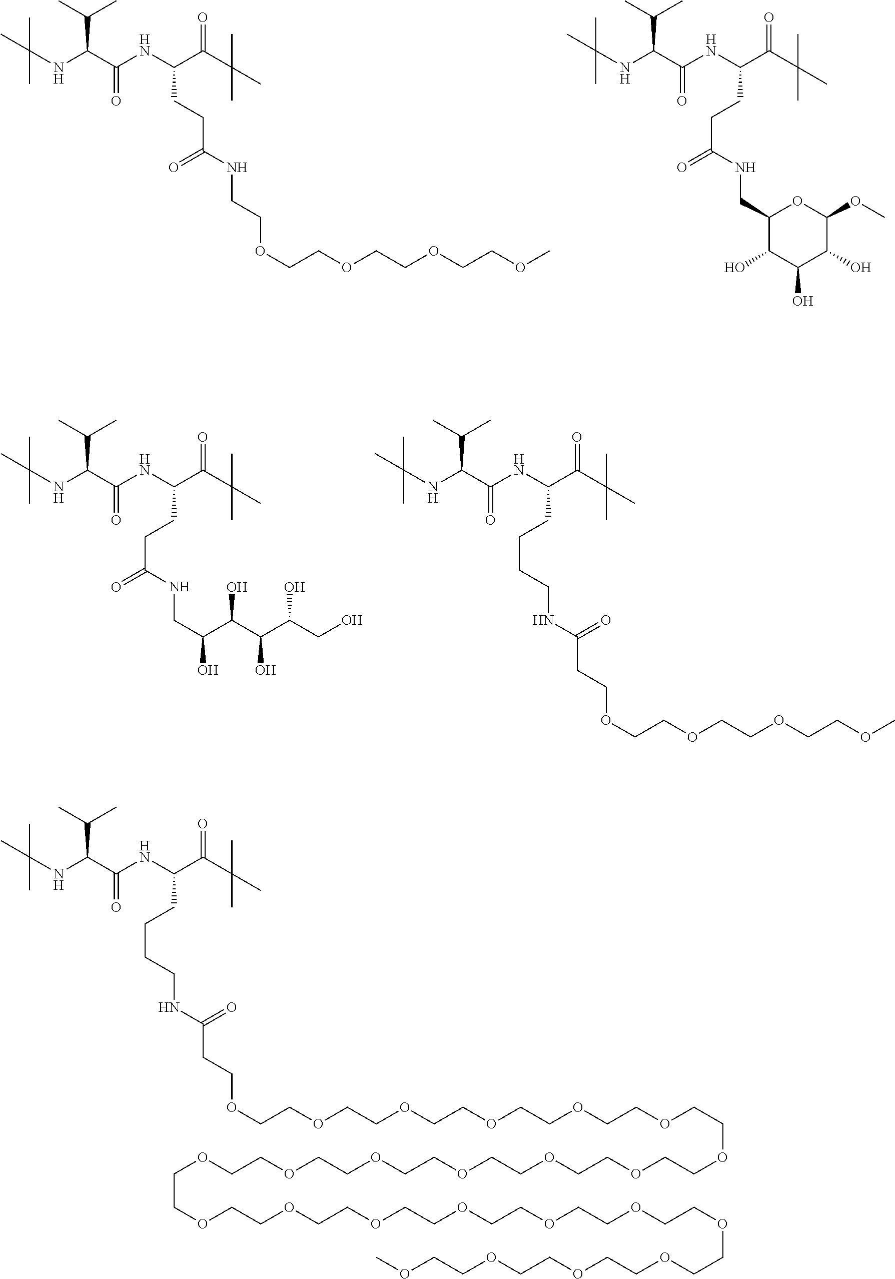

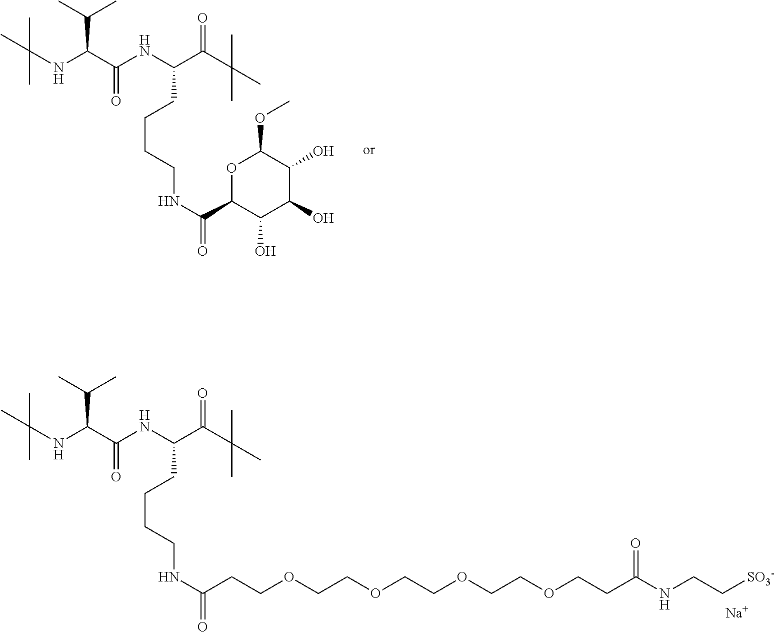

Among the compounds of formula (IV) that are subject matter of the disclosure, a group of compounds is composed of the compounds of the following structure (beta epoxide configuration):

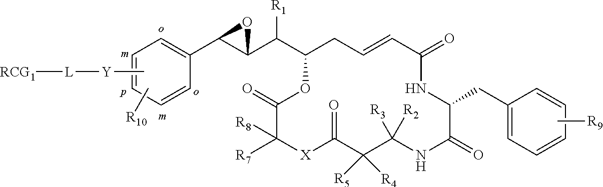

wherein:
R₁, R₂, R₃, R₄, R₅, R₆, R₇, R₈, R₉, R₁₀, R₁₁, R₁₂, n, RCG₁, X, Y and L are as defined in formula (IV).

All these sub-groups taken alone or in combination are part of the present disclosure.

According to at least one embodiment, the present disclosure relates to compounds of formula (IV) wherein:
RCG1 is COOH,

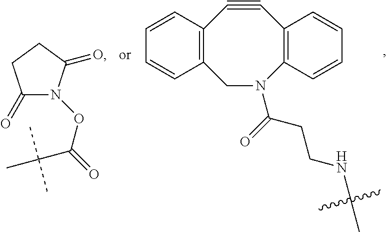

L represents one of the below compounds of formula (II):

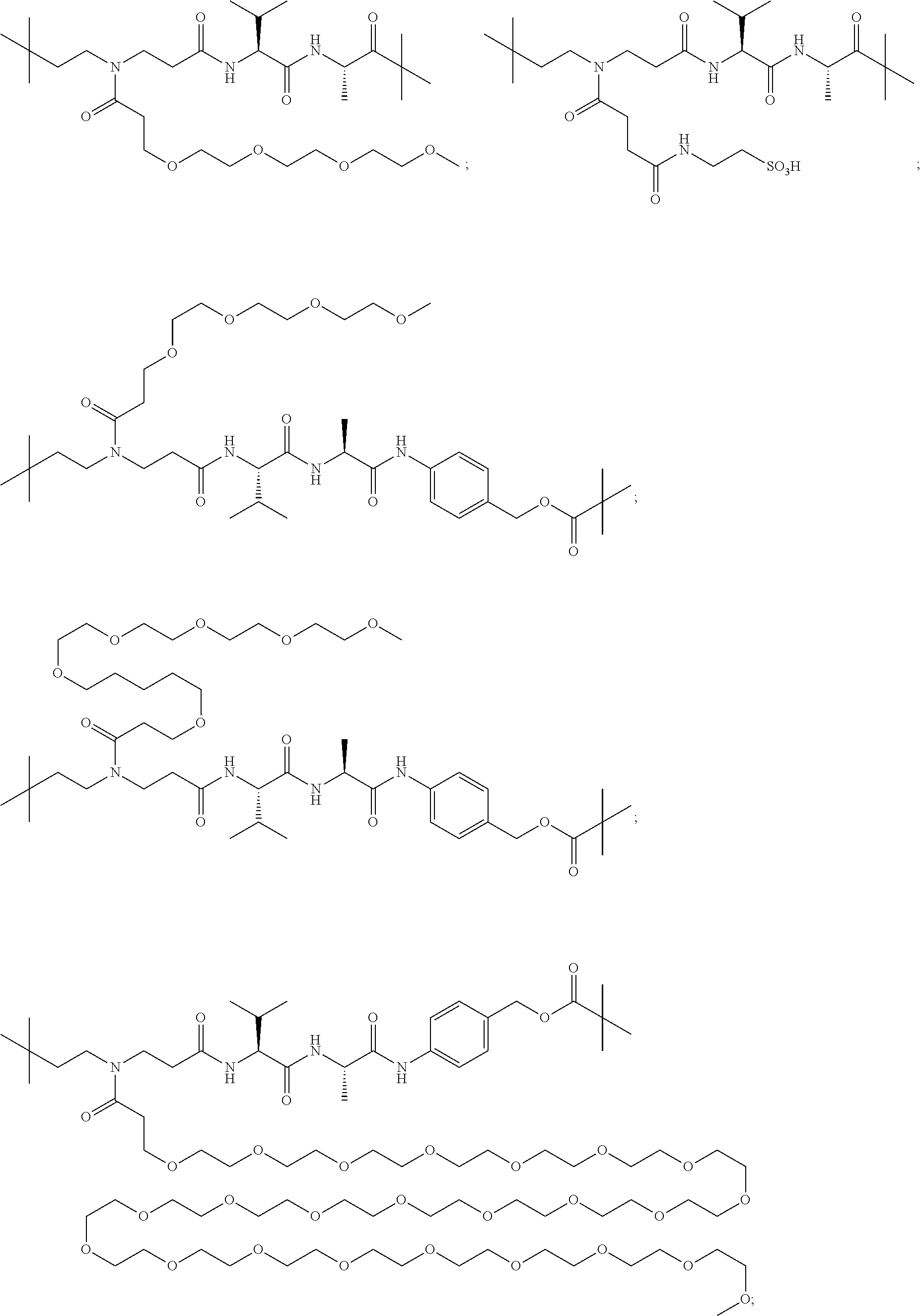

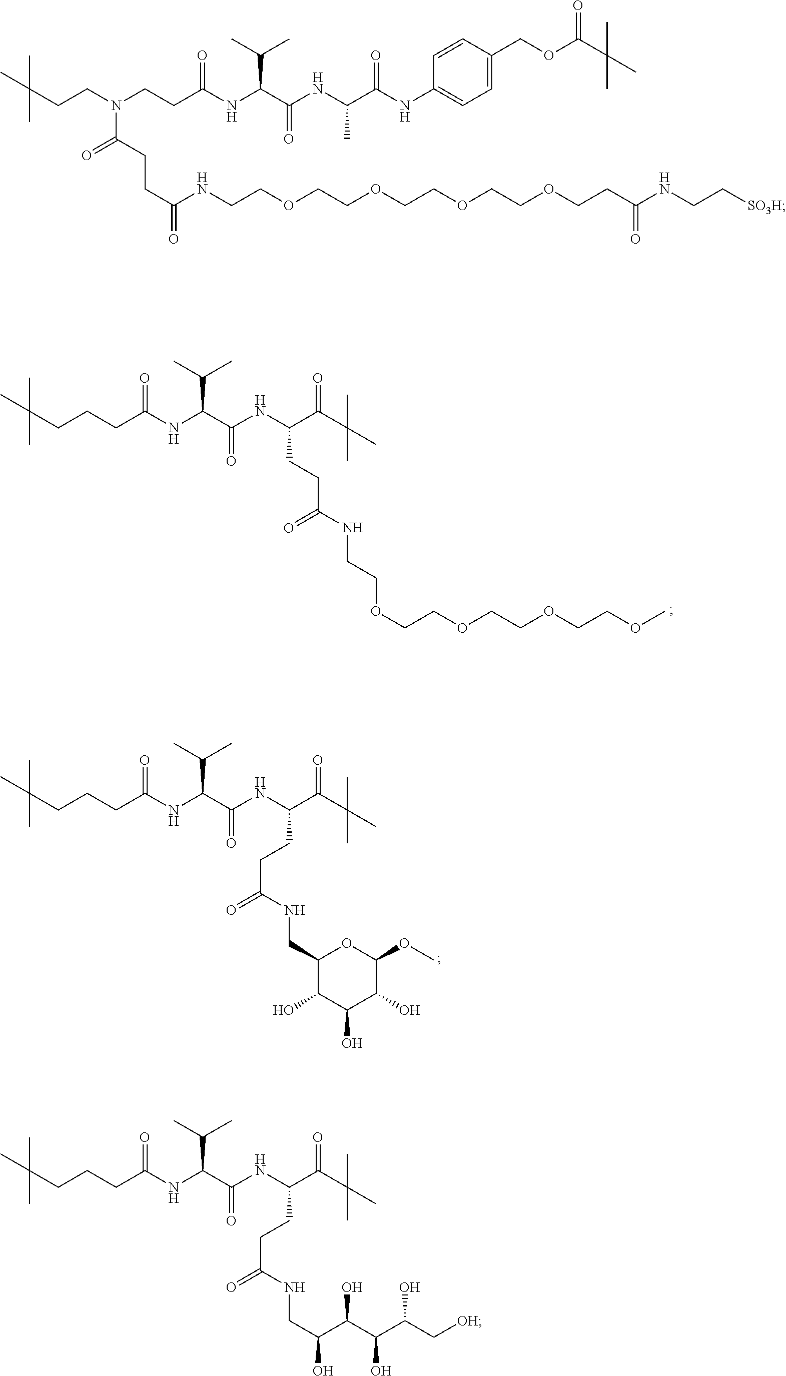

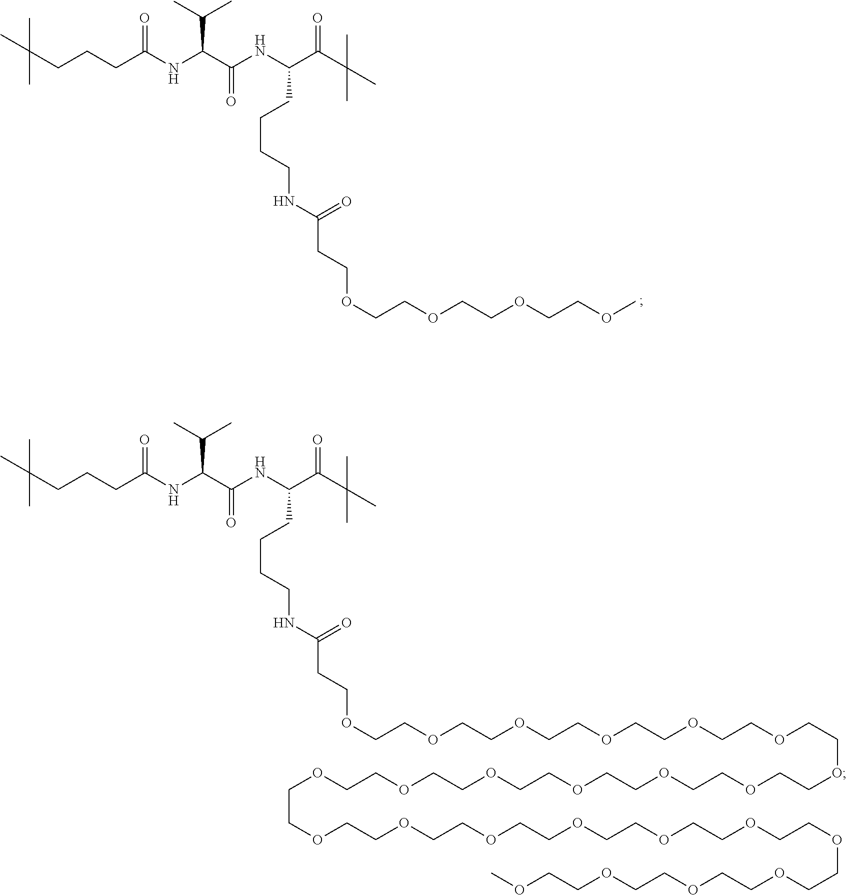

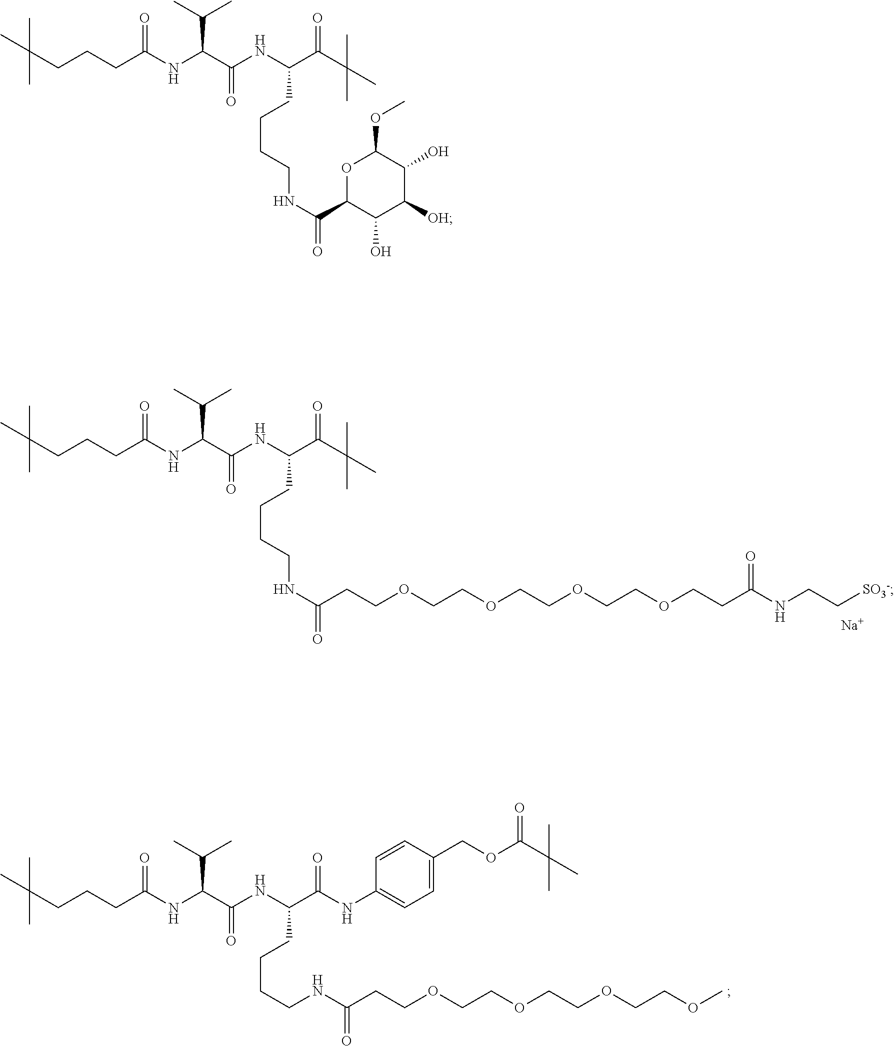

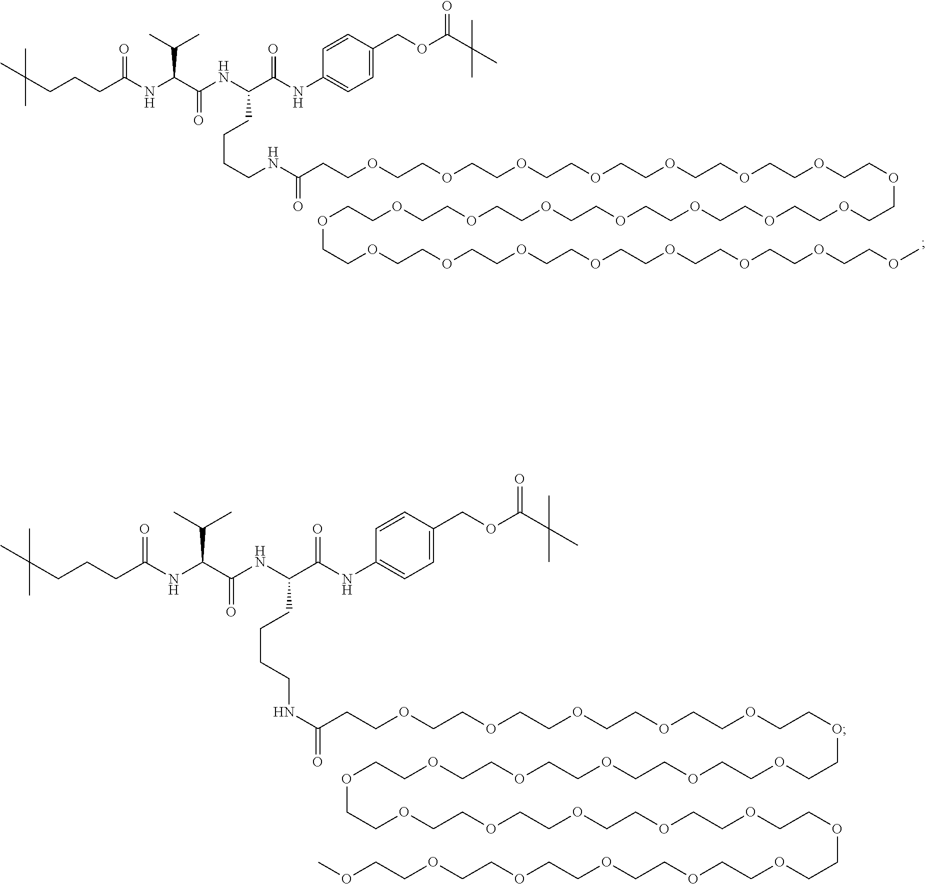

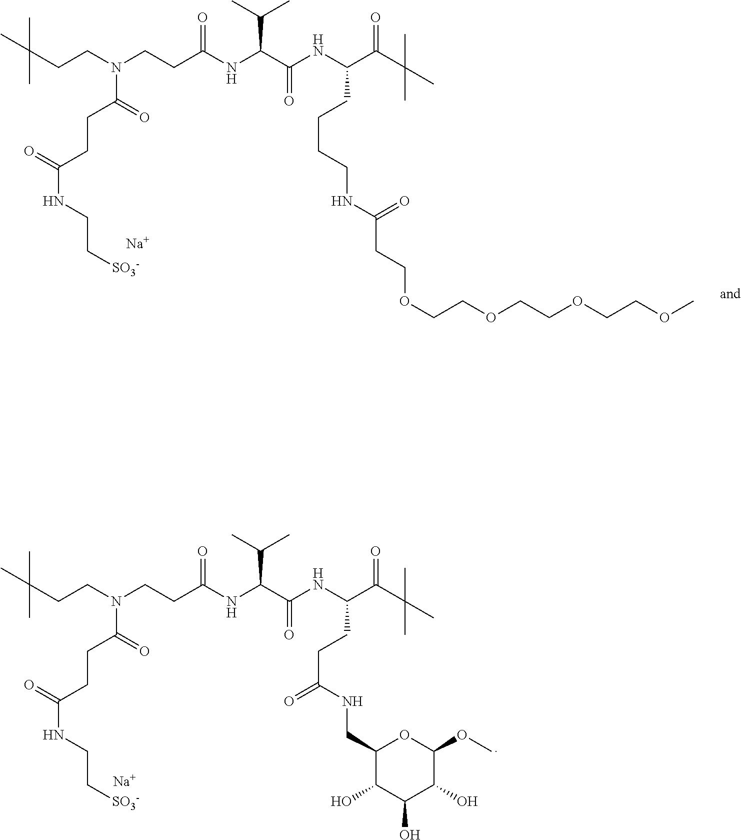

Y is a NR₁₁—(C₁₋₆)alkyl in which R₁₁ is a hydrogen atom, such as Y is NH—CH₂;
R₁ represents a (C₁-C₆)alkyl group, such as a methyl group;
R₂ and R₃ represent independently of each other a hydrogen atom or a (C₁-C₆)alkyl group such as a methyl group;
R₄ and R₅ represent independently of each other a (C₁-C₆)alkyl group, such as a methyl group;
R₇ and R₈ represent independently of each other a hydrogen atom or a (C₁-C₆)alkyl group, such as an isobutyl group or a neopentyl group, for instance one of R₇ and R₈ represents a (C₁-C₆)alkyl group, for example an isobutyl group or a neopentyl group and the other of R₇ and R₈ represents a hydrogen atom;
X represents an oxygen atom or NH;
R₉ represents two substituents selected from a (C₁-C₄)alkoxy group, such as a methoxy group, and a halogen atom, for instance a chlorine atom, such as R₉ represents 3-Cl and 4-methoxy; and
R₁₀ represents a hydrogen atom.

Among the compounds of formula (IV) that are the subject matter of the present disclosure, mention may be made in particular of the following compounds:

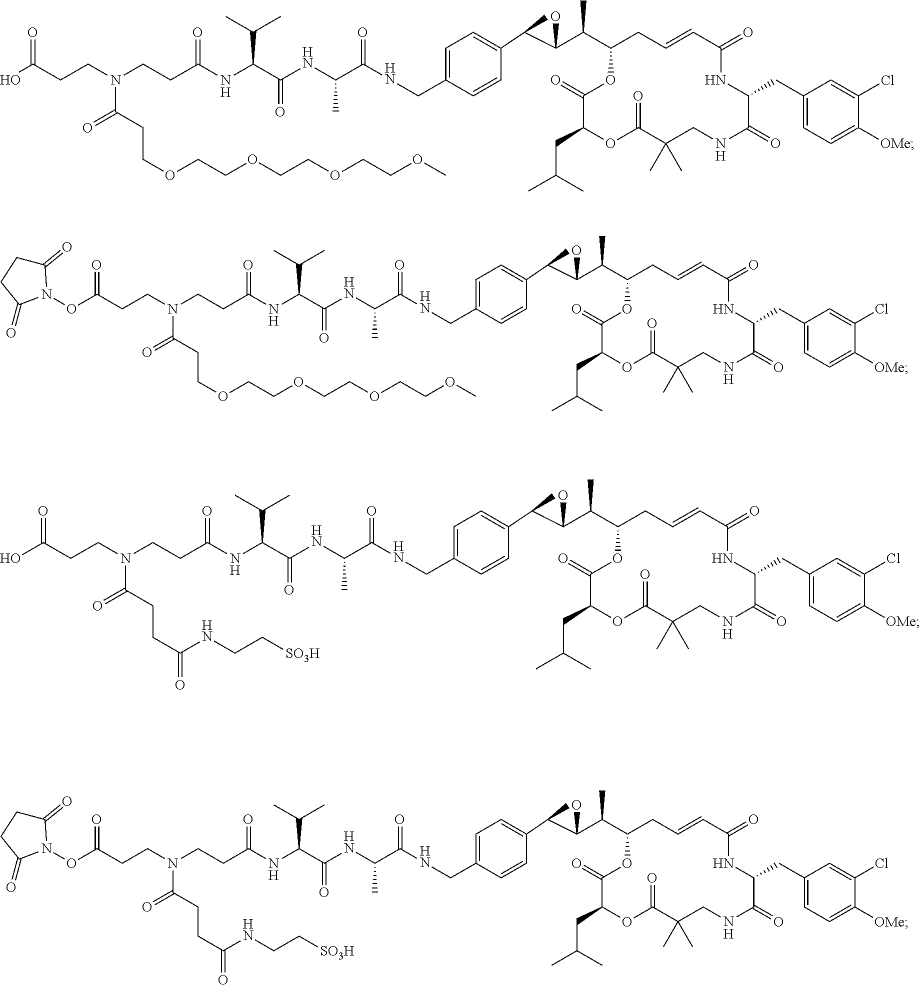

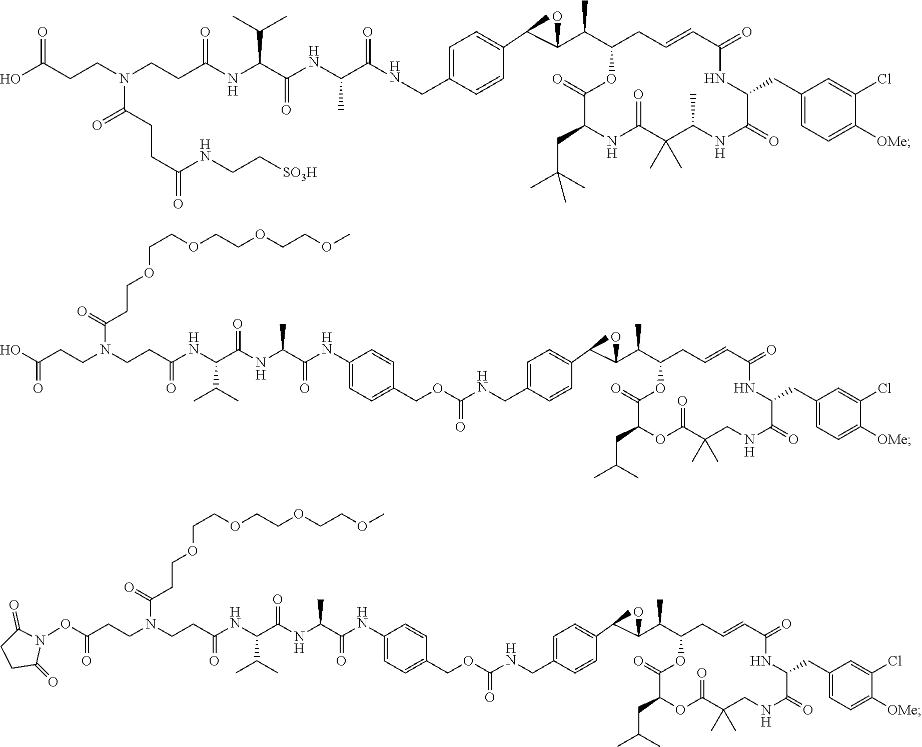

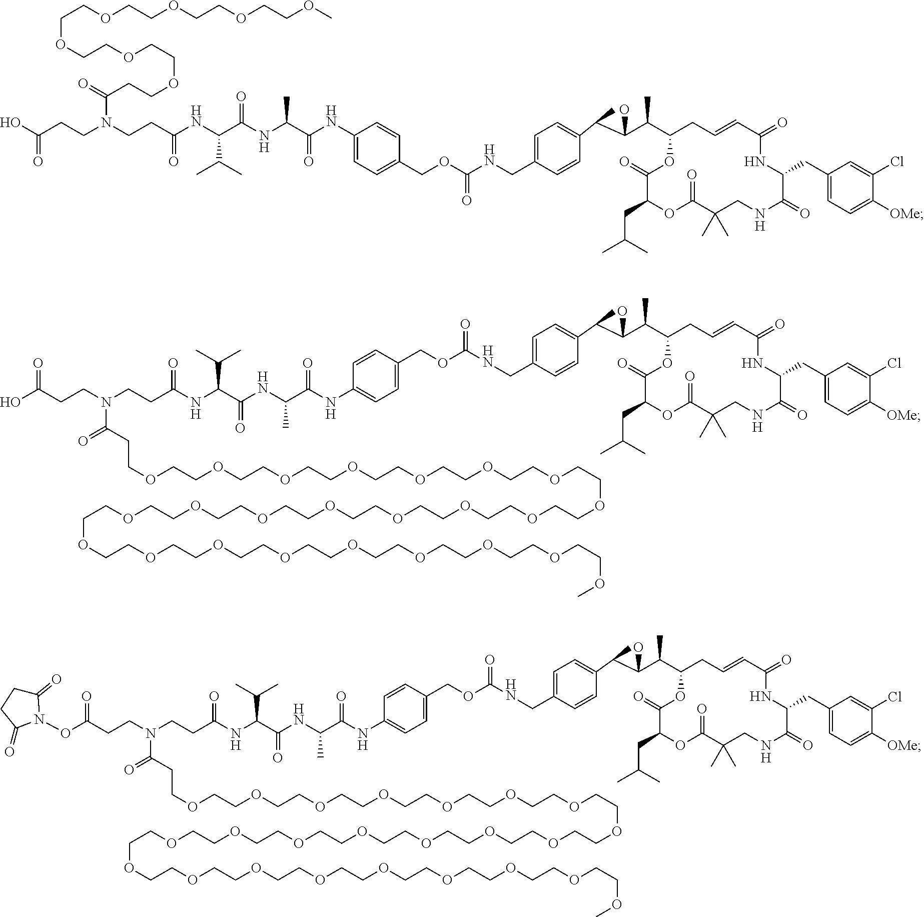

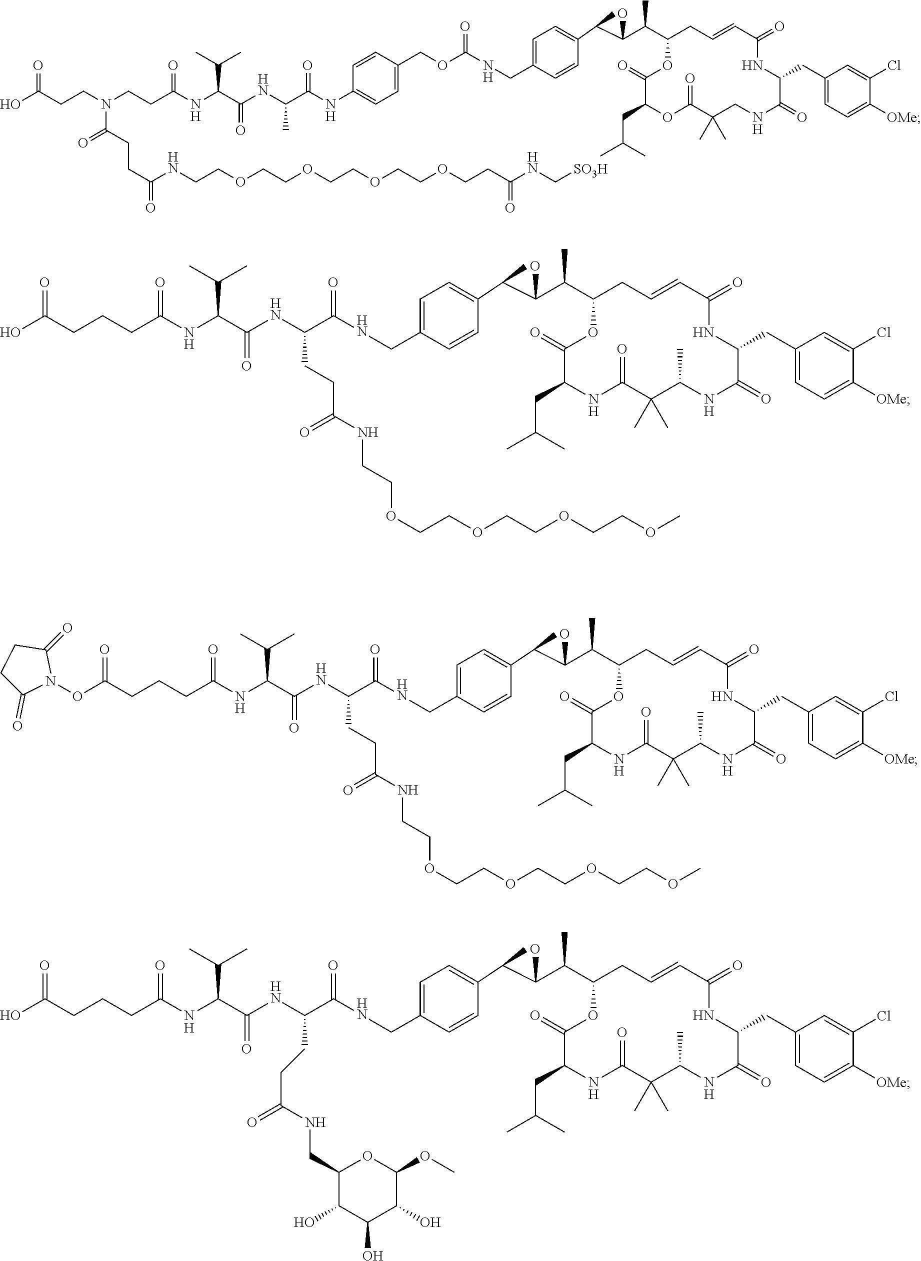

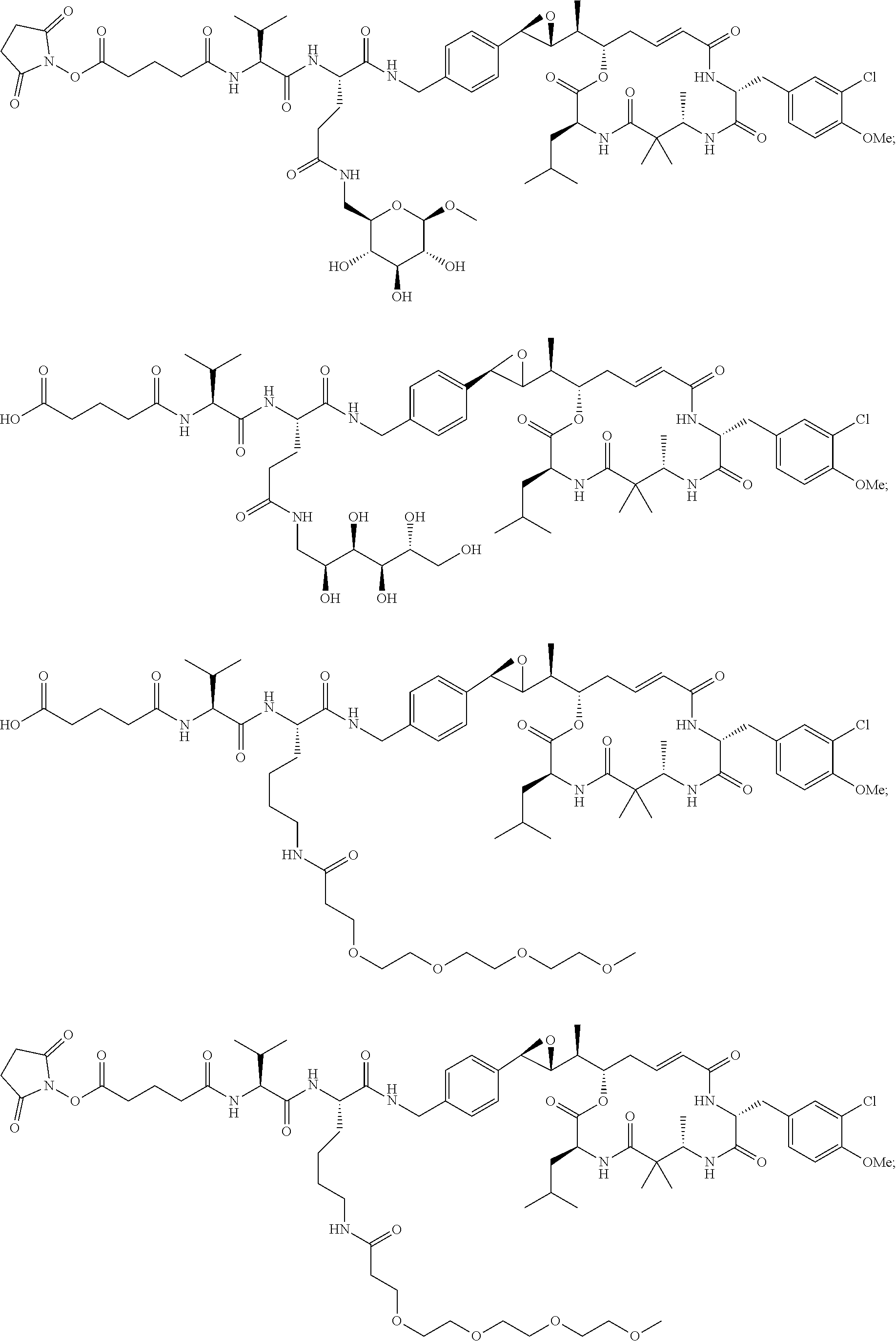

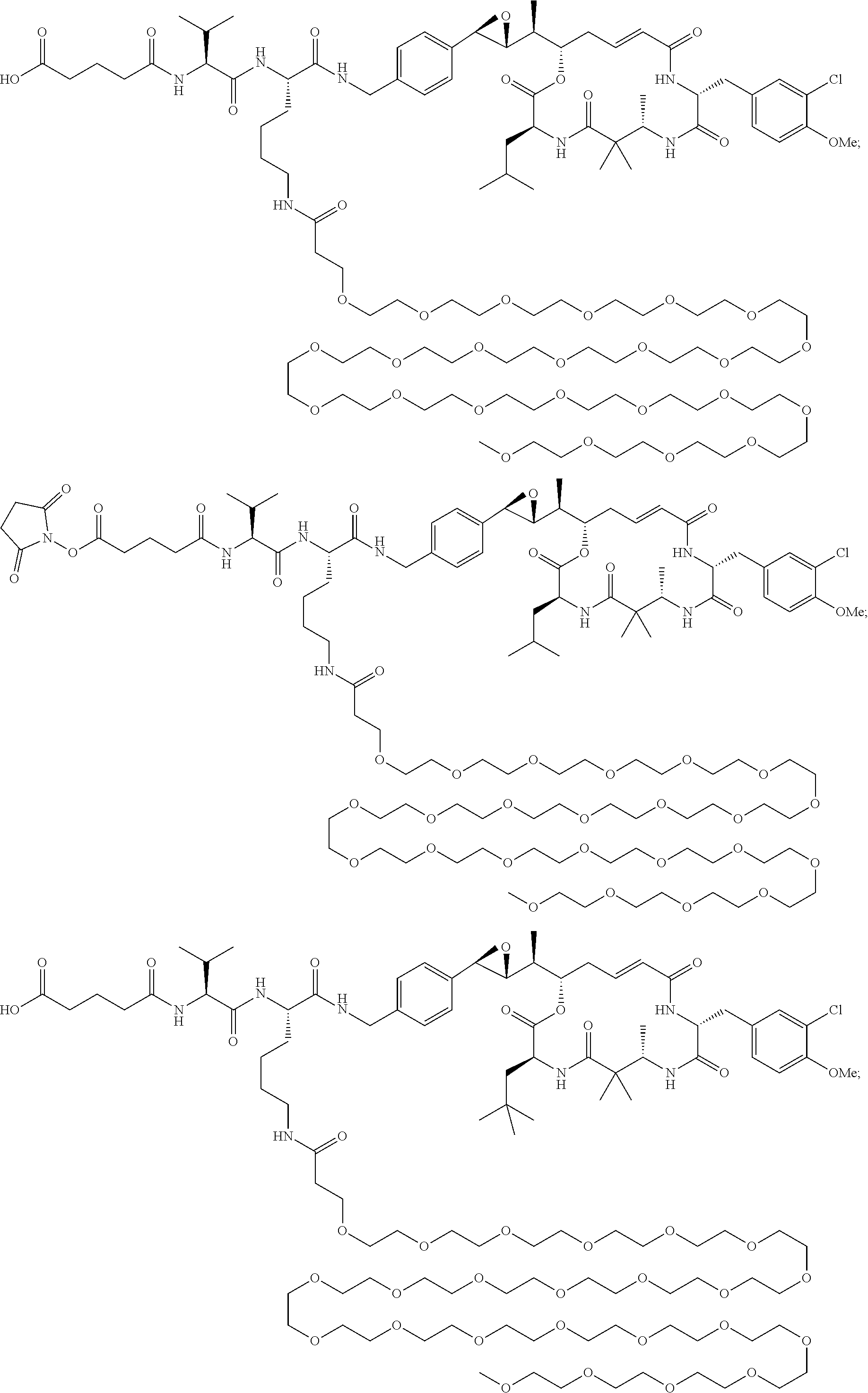

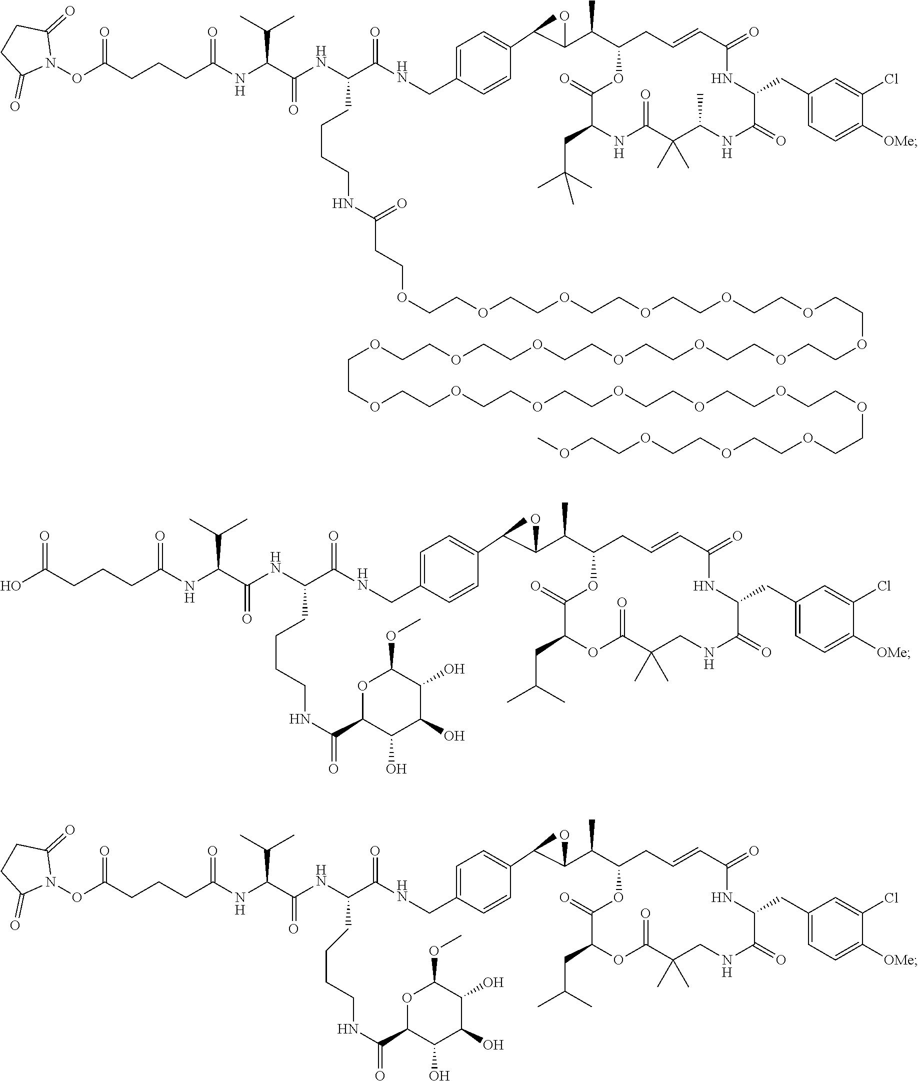

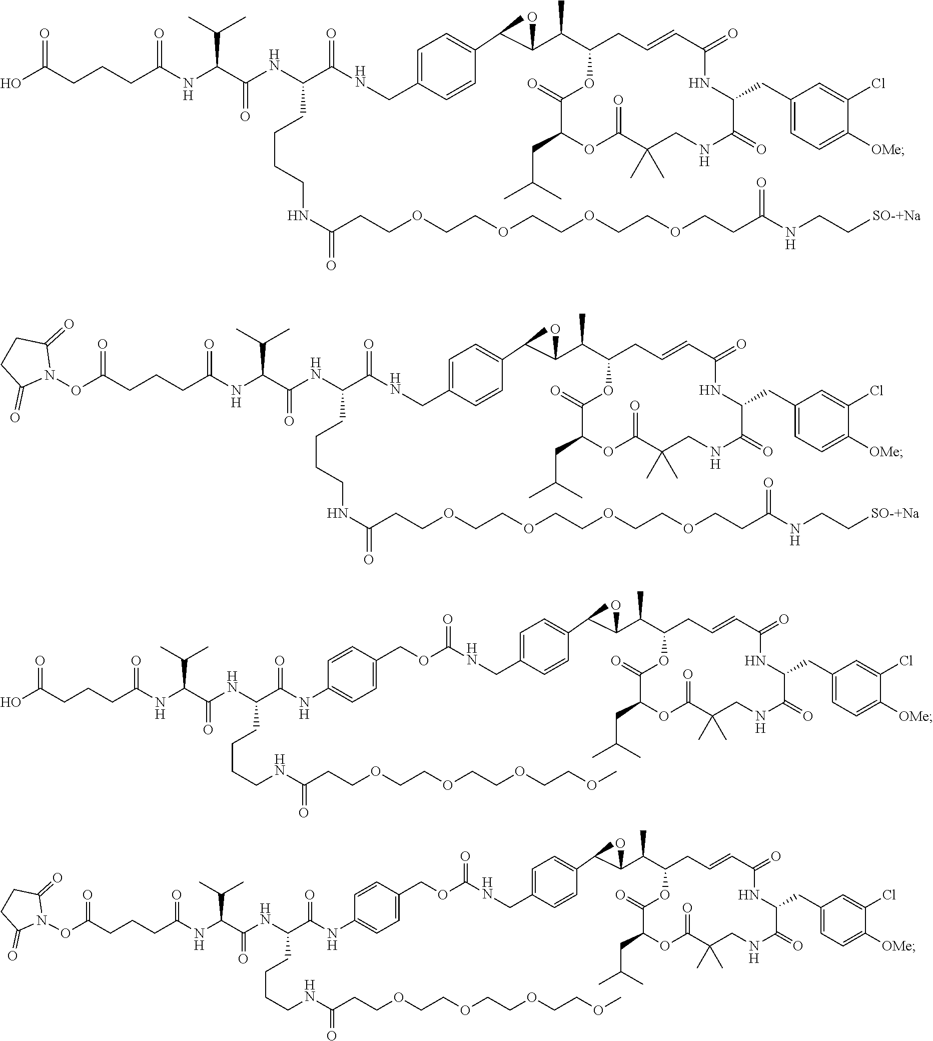

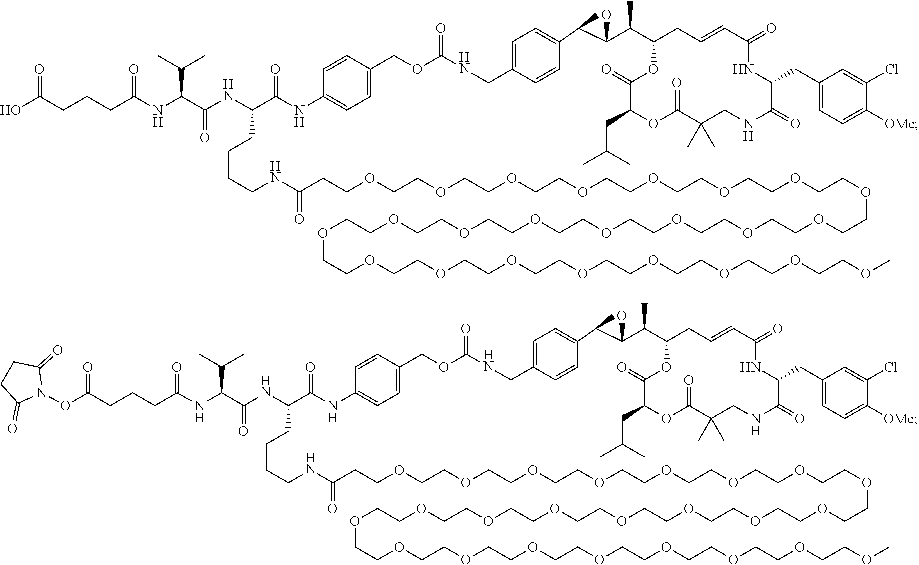

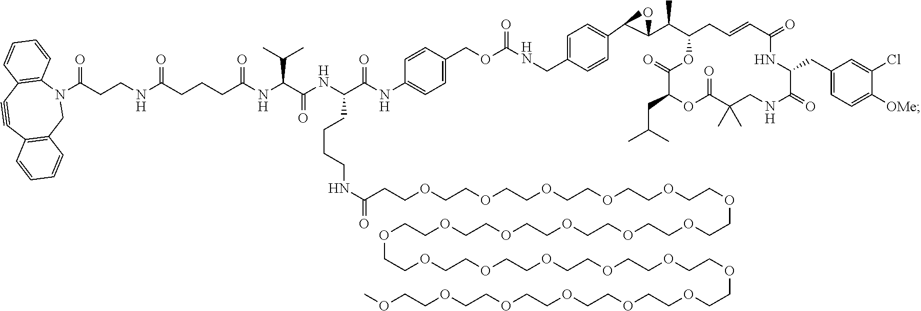 [SMILES: COCCOCCOCCOCCOCCOCCOCCOCCOCCOCCOCCOCCOCCOCCOCCOCCOCCOCCOCCOCCOCCOCCOCCOCCC(=O)NCCCC[C@H](NC(=O)[C@@H](NC(=O)CCCC(=O)NCCC(=O)N1Cc2ccccc2C#Cc2ccccc21)C(C)C)C(=O)Nc1ccc(COC(=O)NCc2ccc([C@@H](C)[C@H](O)[C@@H](C)[C@@H]3C/C=C/C(=O)N[C@H](Cc4ccc(OC)c(Cl)c4)C(=O)NCC(C)(C)C(=O)O[C@@H](CC(C)C)C(=O)O3)cc2)cc1]

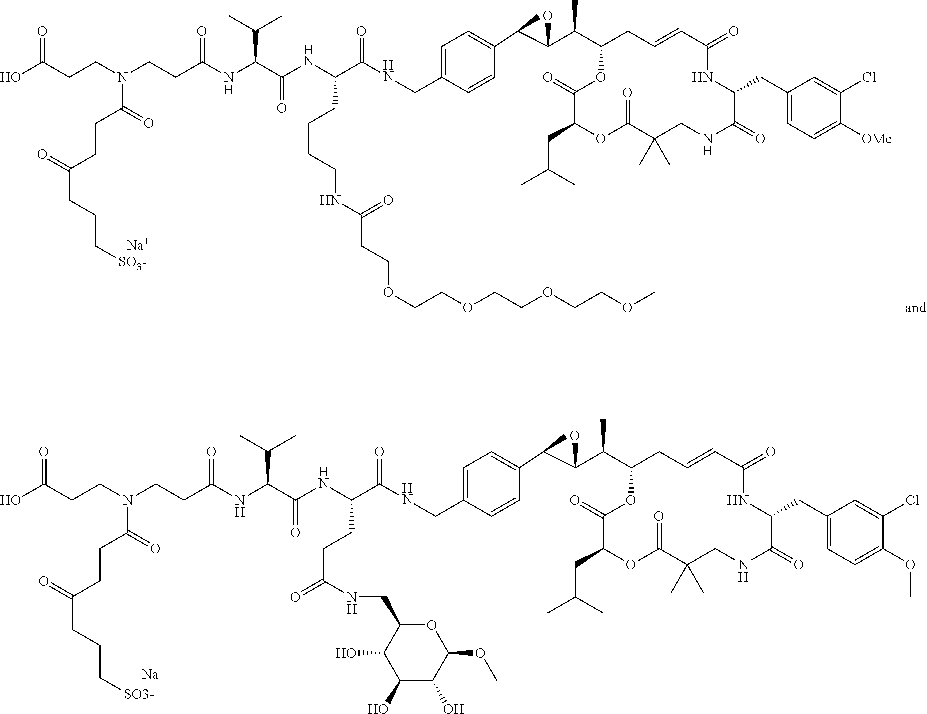

The disclosure further relates to conjugates of formula (V):

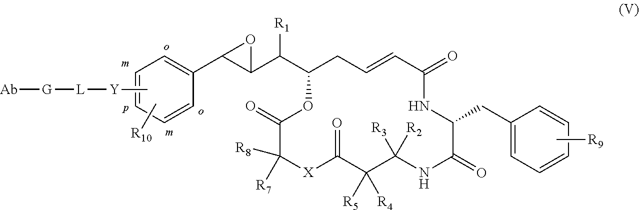

wherein:
R₁, R₂, R₃, R₄, R₅, R₆, R₇, R₈, R₉, R₁₀, R₁₁ and R₁₂ are as defined in formula (IV);
X, Y and L are as defined in formula (IV);
G represents the product of reaction between RCG1, a reactive chemical group present at the end of the linker L of formula (II), and RCG2, an orthogonal reactive chemical group present on a polypeptide such as the antibody (Ab);
Ab represents an antibody.

The attachment between the cryptophycin payload of formula (IV) and the antibody, in order to obtain the conjugate of formula (V), is produced by the reaction between the reactive chemical group RCG1 present on the payload as defined above that is reactive towards a reactive group RCG2 present on the polypeptide such as the antibody. The reaction between RCG1 and RCG2 ensures the attachment of the compound of formula (IV) to the antibody by formation of a covalent bond. In the conjugate of formula (V), parts of RCG1 and RCG2 can remain forming the attachment between the linker and the antibody.

Examples of RCG2 that may be mentioned include (Garnett M. C., et al., Advanced Drug Delivery Reviews 2001, 53, 171-216):
(i) 8-amino groups of lysines borne by the side chains of the lysine residues that are present at the surface of an antibody;
(ii) α-amino groups of N-terminal amino acids of antibody heavy and light chains;
(iii) the saccharide groups of the hinge region;
(iv) the thiols of cysteines generated by reducing intra-chain disulfide bonds or the thiols of engineered cysteines;
(v) amide groups borne by the side chains of some glutamine residues that are present at the surface of an antibody;
(vi) aldehyde groups introduced using formylglycine generating enzyme.

More recently, other conjugation approaches have been considered, for instance the introduction of cysteines by mutation (Junutula J. R., et al., Nature Biotechnology 2008, 26, 925-932), the introduction of unnatural amino acids allowing other types of chemistry (Axup J. Y., et al., PNAS 2012, 109, 40, 16101-16106) or the conjugation on antibody glycans (Zhou Q., et al., Bioconjugate Chem. 2014, 25, 510-520). Use of cysteine bridging dibromomaleimides (Behrens C. R., et al., Mol. Pharmaceutics 2015 3986-3998) and bis-sulfone reagents (Bryant P., et al., Mol. Pharmaceutics 2015 1872-1879) in order to cross-link antibodies have also been described and could be applied to the present disclosure.

Another approach for site-specific modifications of antibodies is based on enzymatic conjugation using for example bacterial transglutaminase (Jeger S., et al., Angew. Chem. Int. Ed. 2010, 49, 9995-9997; Strop P., et al., Chem. Biol. 2013, 20, 161-167) or formylglycine generating enzyme (Hudak J. E., et al., Angew. Chem. Int. Ed. 2012, 51, 4161-4165). For a review of site-specific conjugation strategies, see Agarwal P. and Bertozzi C. R., Bioconjugate Chem 2015, 26, 176-192. These conjugation technologies may also be applied to cryptophycin payloads described in the present disclosure.

It is also possible to chemically modify the polypeptide such as the antibody so as to introduce novel reactive chemical groups RCG2. Thus, it is well known to those skilled in the art how to modify an antibody with the aid of a modifying agent introducing for example activated disulfide, thiol, maleimido, haloacetamido, azido, alkyne or cycloalkyne groups (see especially WO2005/077090 page 14 and WO2011/001052). The modification makes it possible to improve the conjugation reaction and to use a wider variety of RCG1 groups.

For instance, in the case where RCG1 is of the type (ii) above, it is possible to chemically modify the antibody using an adequate modifying agent or to introduce one or more unnatural amino acids so as to introduce the adequate functions RCG2. For example:
when RCG1 represents a N-hydroxysuccinimidyl ester, RCG2 represents a —NH₂ group;
when RCG1 represents a maleimido function, a haloacetamido function, a chorine atom or an activated disulfide, RCG2 may be a SH group;
when RCG1 represents a N₃ group, RCG2 may be a C≡CH function or an activated C≡C function such as a cyclooctyne moiety;
when RCG1 represents a OH group or NH₂ group, RCG2 may be a carboxylic acid or amide function;
when RCG1 represents a SH group, RCG2 may be a maleimido function, a haloacetamido function or an activated disulfide function;
when RCG1 represents a C≡CH function or an activated C≡C function, RCG2 may be a N₃ group;
when RCG1 represents a O-alkyl hydroxylamine function or a Pictet-Spengler reaction substrate, RCG2 may be an aldehyde or ketone function.

Examples of G that may be mentioned include

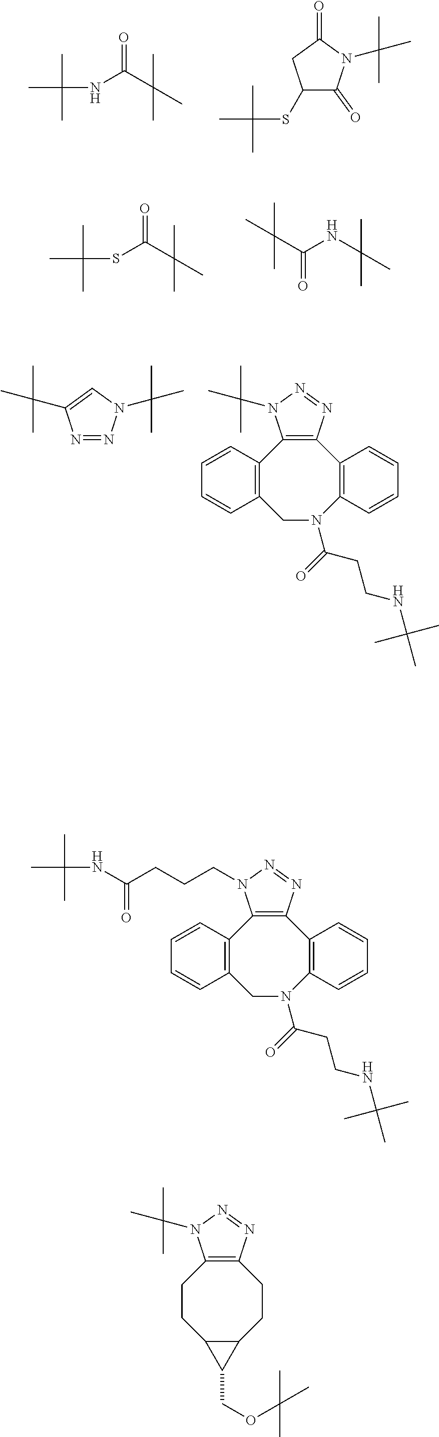

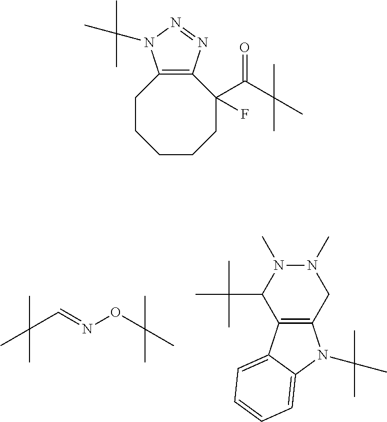 [SMILES: CC(C)(C)C(=O)C1(F)CCCCCc2c1nnn2C(C)(C)C ; CC(C)(C)/C=N/OC(C)(C)C ; CN1Cc2c(c3ccccc3n2C(C)(C)C)C(C(C)(C)C)N1C]

For instance, G represents the following groups:

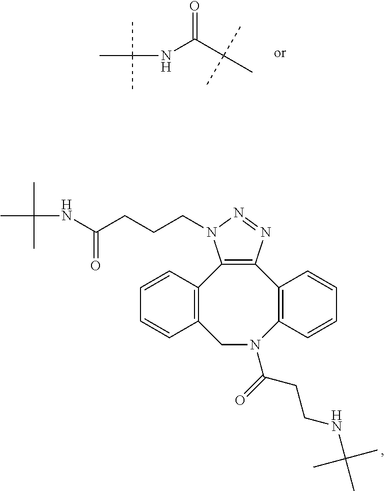

such as

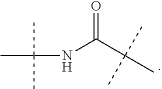

Among the compounds of formula (V) that are subject matter of the present disclosure, a group of compounds is composed of the compounds for which R₁ represents a (C₁-C₆)alkyl, such as a methyl group.

Among the compounds of formula (V) that are subject matter of the disclosure, a group of compounds is composed of the compounds for which each of R₂ and R₃ represents a hydrogen atom.

Among the compounds of formula (V) that are subject matter of the disclosure, a group of compounds is composed of the compounds for which one of R₂ and R₃ represents a (C₁-C₆)alkyl group, such as a methyl group and the other one represents a hydrogen atom.

Among the compounds of formula (V) that are subject matter of the disclosure, a group of compounds is composed of the compounds for which R₂ and R₃ form together with the carbon atom to which they are attached a (C₃-C₆)cycloalkyl group, such as a cyclopropyl group.

Among the compounds of formula (V) that are subject matter of the disclosure, a group of compounds is composed of the compounds for which each of R₄ and R₅ represents a (C₁-C₆)alkyl group, in particular a methyl group.

Among the compounds of formula (V) that are subject matter of the disclosure, a group of compounds is composed of the compounds for which X represents an oxygen atom, that is to say O.

Among the compounds of formula (V) that are subject matter of the disclosure, a group of compounds is composed of the compounds for which X represents NH.

Among the compounds of formula (V) that are subject matter of the disclosure, a group of compounds is composed of the compounds for which R₇ and R₈ represent independently of each other a hydrogen atom or a (C₁-C₆)alkyl group, such as an isobutyl group or a neopentyl group.

Among the compounds of formula (V) that are subject matter of the disclosure, a group of compounds is composed of the compounds for which R₉ represents two substituents selected from a methoxy group and a chlorine atom. For instance, the phenyl nucleus comprises two substituents in positions 3 and 4 on the phenyl nucleus. For example 3-C₁ and 4-methoxy.

Among the compounds of formula (V) that are subject matter of the disclosure, a group of compounds is composed of the compounds for which R₁₀ represents a hydrogen atom.

Among the compounds of formula (v) that are subject matter of the disclosure, a group of compounds is composed of the compounds for which Y is positioned in the para position of the phenyl nucleus.

Among the compounds of formula (V) that are subject matter of the disclosure, a group of compounds is composed of the compounds for which Y NR₁₁—(C₁-C₆)alkyl, such as NR₁₁—(C₁-C₃)alkyl, for instance NH—CH₂.

Among the compounds of formula (V) that are subject matter of the disclosure, a group of compounds is composed of the compounds for which L of formula (II) comprises at least one substituted amino acid AAₛ in the sequence of w amino acids (AA)w, L1 and L2 are as defined in formula (II).

Among the compounds of formula (V) that are subject matter of the disclosure, a group of compounds is composed of the compounds for which L of formula (II) comprises a sequence of w non-substituted amino-acid AAₙₛ, L1 and L2 are as defined in formula (II).

Among the compounds of formula (V) that are subject matter of the disclosure, a group of compounds is composed of the compounds for which (AA)w in L of formula (II) contains at least one substituted amino acid AAₛ and L2 represents:
a (C₁-C₆)alkyl group, such as a (CH₂)₃ group;
a C(═O)—(C₁-C₆)alkyl group, such as a C(═O)—(CH₂)₃ group; or
a (C₁-C₆)alkyl-NA₇-(C₁-C₆)alkyl group, such as a (CH₂)₂—NA₇-(CH₂)₂ group in which A₇ is as defined above.

Among the compounds of formula (V) that are subject matter of the disclosure, a group of compounds is composed of the compounds for which (AA)w in L of formula (II) contains at least one substituted amino acid AAₛ and L2 represents:
a (C₁-C₆)alkyl group, such as a (CH₂)₃ group;
a C(═O)—(C₁-C₆)alkyl group, such as a C(═O)—(CH₂)₃ group; or
a (C₁-C₆)alkyl-NA₇-(C₁-C₆)alkyl group, such as a (CH₂)₂—NA₇-(CH₂)₂ group in which A₇ is a —C(═O)—(CH₂)₂—C(═O)—NH—(CH₂)₂—SO₃H group;
   being understood that each A₇ comprising a SO₃H function can be under salt forms such as alkali metal salts, for instance sodium salts (SO₃^{- +}Na).

Among the compounds of formula (V) that are subject matter of the disclosure, a group of compounds is composed of the compounds for which (AA)w in L of formula (II) contains w non-substituted amino acid AAₙₛ and L2 represents:
a (C₁-C₆)alkyl-NA₇-(C₁-C₆)alkyl group, such as a (CH₂)₂—NA₇-(CH₂)₂ group in which A₇ is as defined above.

Among the compounds of formula (V) that are subject matter of the disclosure, a group of compounds is composed of the compounds for which (AA)w in L of formula (II) contains w non-substituted amino acid AAₙₛ and L2 represents:
a (C₁-C₆)alkyl-NA₇-(C₁-C₆)alkyl group, such as a (CH₂)₂—NA₇-(CH₂)₂ group in which A₇ represents:
   a C(═O)—[(CH₂)₂—O]ₐ—CH₃ group wherein “a” represents an integer ranging from 1 to 50, for instance ranging from 1 to 24, such as 4, 7 and 24, for example A₇ is a C(═O)—[(CH₂)₂—O]₄—CH₃ group, a C(═O)—[(CH₂)₂—O]₇—CH₃ group, or a C(═O)—[(CH₂)₂—O]₂₄—CH₃ group;
   a —C(═O)—(CH₂)₂—C(═O)—NH—(CH₂)₂—SO₃H group; or
   a C(═O)—(CH₂)₂—C(═O)—NH—[(CH₂)₂—O]ₐ—(CH₂)₂—C(═O)—NH—(CH₂)₂—SO₃H group wherein “a” represents an integer ranging from 1 to 50, such as ranging from 1 to 24, for example 4, such as A₇ is —C(═O)—(CH₂)₂—C(═O)—NH—[(CH₂)₂—O]₄—(CH₂)₂—C(═O)—NH—(CH₂)₂—SO₃H group;
      being understood that each A₇ comprising a SO₃H function can be under salt forms such as alkali metal salts, for instance sodium salts (SO₃^{- +}Na);

Among the compounds of formula (V) that are subject matter of the disclosure, a group of compounds is composed of the compounds for which (AA)w in L of formula (II) contains at least one substituted amino acid AAₛ and/or w non-substituted amino acid AAₙₛ and L2 represents:
a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a C(═O)—(C₁-C₆)alkyl-(OCH₂CH₂)ᵢ—NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-NA₇C(═O)—(C₁-C₆)alkyl-(OCH₂CH₂)ᵢ group, a NA₈-(C₁-C₆)alkyl-C(═O)NA₇-(C₁-C₆)alkyl group, a NA₈-(C₁-C₆)alkyl-C(═O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ—NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-NA₇C(═O)—(C₁-C₆)alkyl-(OCH₂CH₂)ᵢ group, a C(═O)—NA₈-(C₁-C₆)alkyl-C(═O)NA₇-(C₁-C₆)alkyl group, a C(═O)—NA₈-(C₁-C₆)alkyl-C(═O)NA₇-(C₁-C₆)alkyl-(OCH₂CH₂)ᵢ group, a C(═O)—NA₈-(C₁-C₆)alkyl-NA₇-(C₁-C₆)alkyl group, a C(═O)—NA₈-(C₁-C₆)alkyl-NA₇-(C₁-C₆)alkyl-(OCH₂CH₂)ᵢ group or a C(═O)—NA₈-(C₁-C₆)alkyl-(OCH₂CH₂)ᵢ—NA₇-(C₁-C₆)alkyl group; wherein i, A₇ and A₈ are as defined above.

Among the compounds of formula (V) that are subject matter of the disclosure, a preferred group of compounds is composed of the compounds for which (AA)w in L of formula (II) contains at least one substituted amino acid AAₛ and/or w non-substituted amino acid AAₙₛ and L2 represents:
a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—NA₇-aryl group, a C(═O)—NA₇-heteroaryl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-C(═O)NA₇-(C₁-C₆)alkyl group, a NA₈-(C₁-C₆)alkyl-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-C(═O)NA₇-(C₁-C₆)alkyl group, or a C(═O)—NA₈-(C₁-C₆)alkyl-NA₇-(C₁-C₆)alkyl group; wherein i, A₇ and A₈ are as defined above.

Among the compounds of formula (V) that are subject matter of the disclosure, a more preferred group of compounds is composed of the compounds for which (AA)w in L of formula (II) contains at least one substituted amino acid AAₛ and/or w non-substituted amino acid AAₙₛ and L2 represents:
a NA₇-(C₁-C₆)alkyl group, a (C₁-C₆)alkyl-NA₇ group, a NA₇-(CH₂CH₂O)ᵢ(C₁-C₆)alkyl group, a (C₁-C₆)alkyl-NA₇C(═O)—(C₁-C₆)alkyl group, a (C₁-C₆)alkyl-NA₇C(═O)—(C₁-C₆)alkyl-(OCH₂CH₂)ᵢ group, a (C₁-C₆)alkyl-C(═O)NA₇-(C₁-C₆)alkyl group, a (C₁-C₆)alkyl-C(═O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(═O)—NA₇-(C₁-C₆)alkyl group, a C(═O)—(C₁-C₆)alkyl-NA₇ group, a C(═O)—NA₇-(CH₂CH₂O)ᵢ(C₁-C₆)alkyl group, a C(═O)—(C₁-C₆)alkyl-NA₇C(═O)—(C₁-C₆)alkyl group, a C(═O)—(C₁-C₆)alkyl-NA₇C(═O)—(C₁-C₆)alkyl-(OCH₂CH₂)ᵢ group, a C(═O)—(C₁-C₆)alkyl-C(═O)NA₇-(C₁-C₆)alkyl group, a C(═O)—(C₁-C₆)alkyl-C(═O)NA₇-(C₁-C₆)alkyl-(OCH₂CH₂)ᵢ group, a C(═O)—(C₁-C₆)alkyl-NA₇-(C₁-C₆)alkyl group, a C(═O)—(C₁-C₆)alkyl-NA₇-(C₁-C₆)alkyl-(OCH₂CH₂)ᵢ group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-C(═O)NA₇-(C₁-C₆)alkyl group, a NA₈-(C₁-C₆)alkyl-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-C(═O)NA₇-(C₁-C₆)alkyl group, or a C(═O)—NA₈-(C₁-C₆)alkyl-NA₇-(C₁-C₆)alkyl group; wherein i, A₇ and A₈ are as defined above.

Among the compounds of formula (V) that are subject matter of the disclosure, a still more preferred group of compounds is composed of the compounds for which (AA)w in L of formula (II) contains at least one substituted amino acid AAₛ and/or w non-substituted amino acid AAₙs and L2 represents:
a NA₇-(C₁-C₆)alkyl group, a (C₁-C₆)alkyl-NA₇ group, a (C₁-C₆)alkyl-NA₇C(═O)—(C₁-C₆)alkyl group, a (C₁-C₆)alkyl-C(═O)NA₇-(C₁-C₆)alkyl group, a (C₁-C₆)alkyl-NA₇-(C₁-C₆)alkyl group, a C(═O)—NA₇-(C₁-C₆)alkyl group, a C(═O)—(C₁-C₆)alkyl-NA₇ group, a C(═O)—(C₁-C₆)alkyl-NA₇C(═O)—(C₁-C₆)alkyl group, a C(═O)—(C₁-C₆)alkyl-C(═O)NA₇-(C₁-C₆)alkyl group, a C(═O)—(C₁-C₆)alkyl-NA₇-(C₁-C₆)alkyl group, a NA₈-(C₁-C₆)alkyl-NA₇ group, a NA₈-(C₁-C₆)alkyl-NA₇C(═O)—(C₁-C₆)alkyl group, a NA₈-(C₁-C₆)alkyl-C(═O)NA₇-(C₁-C₆)alkyl group, a NA₈-(C₁-C₆)alkyl-NA₇-(C₁-C₆)alkyl group, a C(═O)—NA₈-(C₁-C₆)alkyl-NA₇ group, a C(═O)—NA₈-(C₁-C₆)alkyl-NA₇C(═O)—(C₁-C₆)alkyl group, a C(═O)—NA₈-(C₁-C₆)alkyl-C(═O)NA₇-(C₁-C₆)alkyl group, or a C(═O)—NA₈-(C₁-C₆)alkyl-NA₇-(C₁-C₆)alkyl group; wherein A₇ and A₈ are as defined above.

Among the compounds of formula (V) that are subject matter of the disclosure, a most preferred group of compounds is composed of the compounds for which (AA)w in L of formula (II) contains at least one substituted amino acid AAₛ and/or w non-substituted amino acid AAₙₛ and L2 represents:
a (C₁-C₆)alkyl-NA₇-(C₁-C₆)alkyl group; wherein A₇ is as defined above.

Among the compounds of formula (V) that are subject matter of the disclosure, a group of compounds is composed of the compounds for which (AA)w in L of formula (II) contains at least one substituted amino acid AAₛ and/or w non-substituted amino acid AAₙₛ and L2 comprises a A₇ representing:
a C(═O)—[(CH₂)₂—O]₄—CH₃ group; a C(═O)—[(CH₂)₂—O]₇—CH₃ group; a C(═O)—[(CH₂)₂—O]₂₄—CH₃ group; a C(═O)—(CH₂)₂—C(═O)—NH—(CH₂)₂—SO₃H group; a C(═O)—(CH₂)₂—C(═O)—NH—[(CH₂)₂—O]₄—(CH₂)₂—C(═O)—NH—(CH₂)₂—SO₃H group;
   being understood that each A₇ comprising a SO₃H function can be under salt forms such as alkali metal salts, for instance sodium salts (SO₃^{- +}Na).

Among the compounds of formula (V) that are subject matter of the disclosure, a group of compounds is composed of the compounds for which (AA)w in L of formula (II) contains at least one substituted amino acid AAₛ and/or w non-substituted amino acid AAₙₛ and L2 represents:

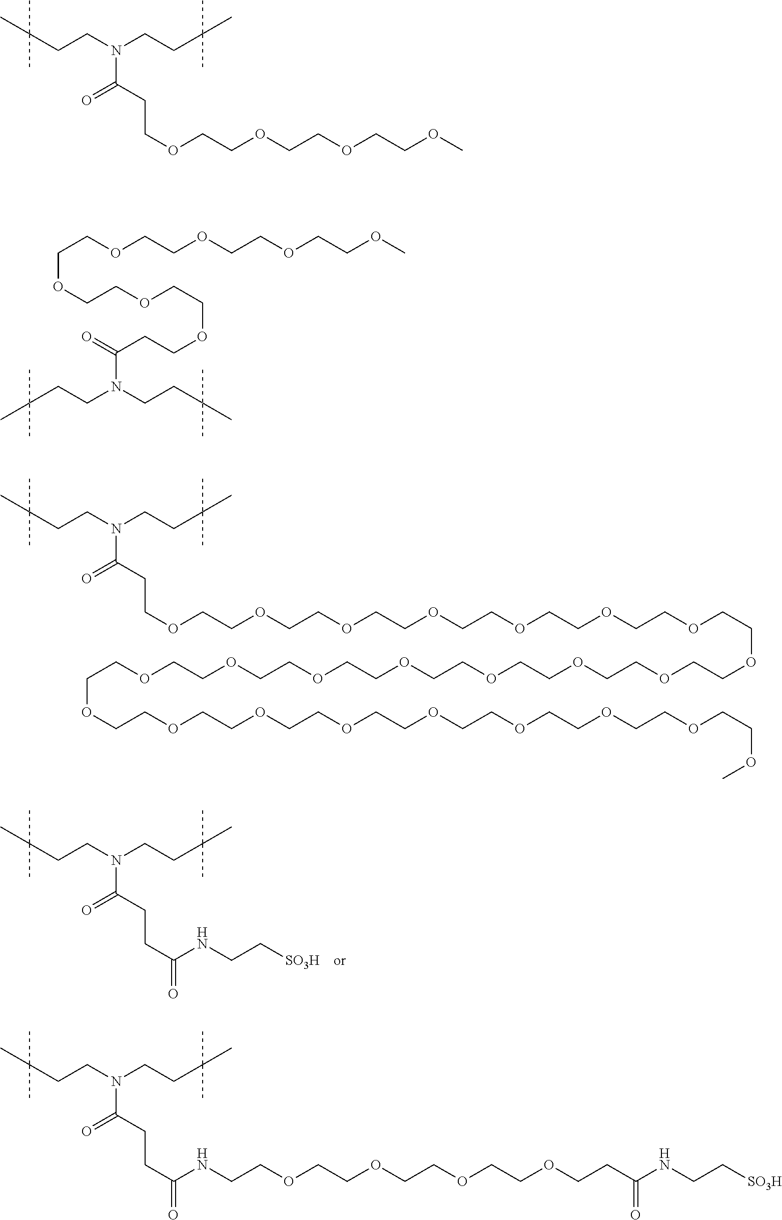

Among the compounds of formula (V) that are subject matter of the disclosure, a group of compounds is composed of the compounds comprising in L a sequence (AA_{S})w containing at least one substituted amino acid AA_{S}, (AA_{S})w being selected from the list:

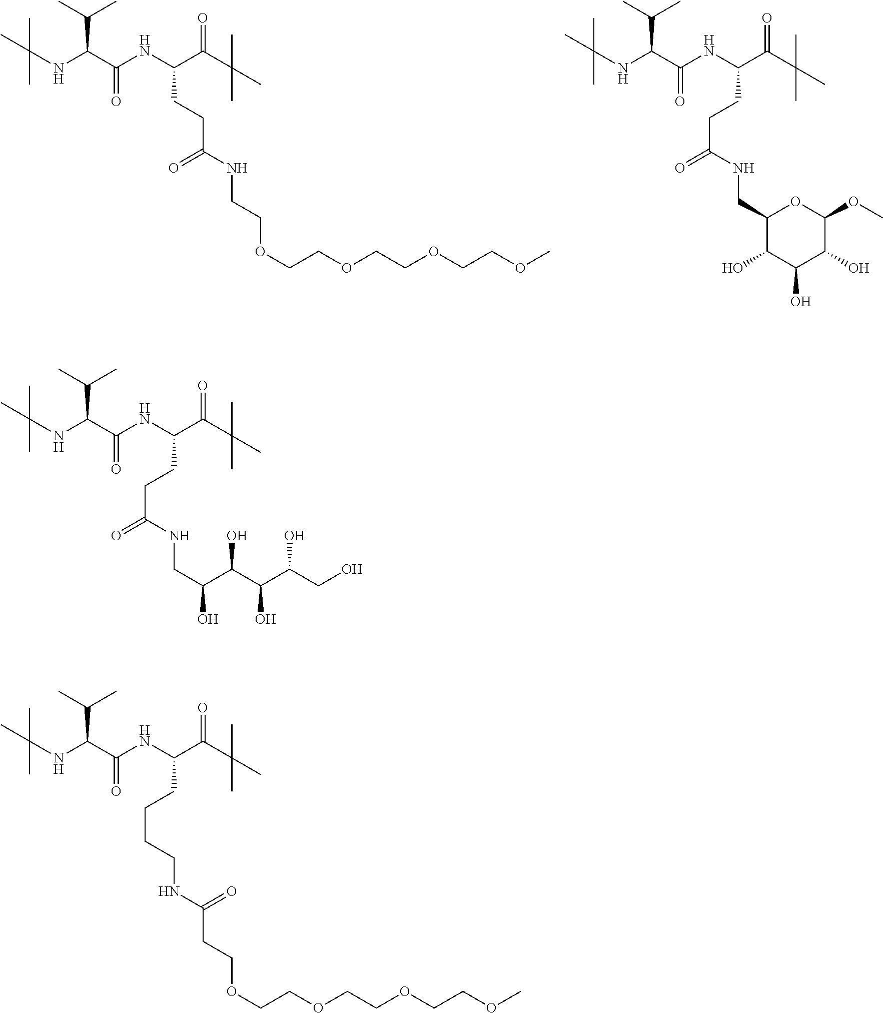

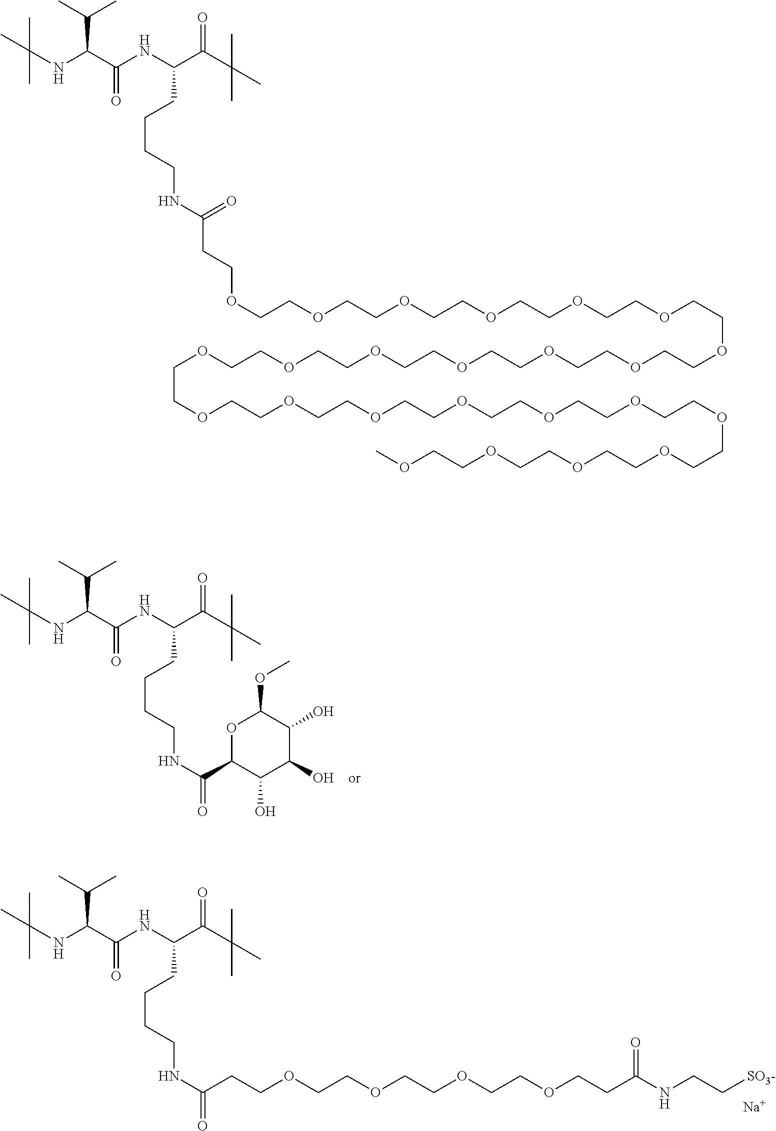

Among the compounds of formula (V) that are subject matter of the disclosure, a group of compounds is composed of the compounds for which Ab is an anti-Epha2 antibody, for instance hu2H11_R35R74 antibody as represented by SEQ ID NO: 1 (light chain of antibody hu2H11_R35R74) and by SEQ ID NO: 2 (heavy chain of antibody hu2H11_R35R74) corresponding to respectively SEQ ID NO: 16 and SEQ ID NO:18 represented in WO2011039724 A1.

According to at least one embodiment, the present disclosure relates to compounds of formula (V) wherein:
Ab represents an antibody;
G represents:

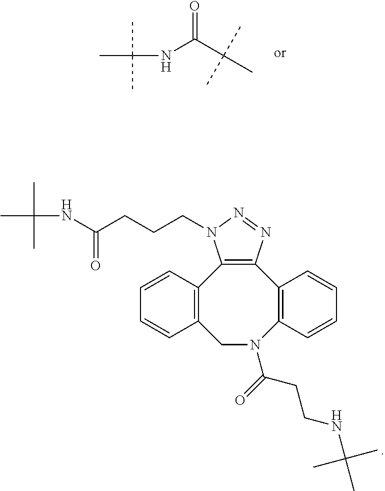

Y is a NR₁₁—(C₁₋₆)alkyl group in which R₁₁ is a hydrogen atom, for instance Y is a NH—CH₂ group;
L represents

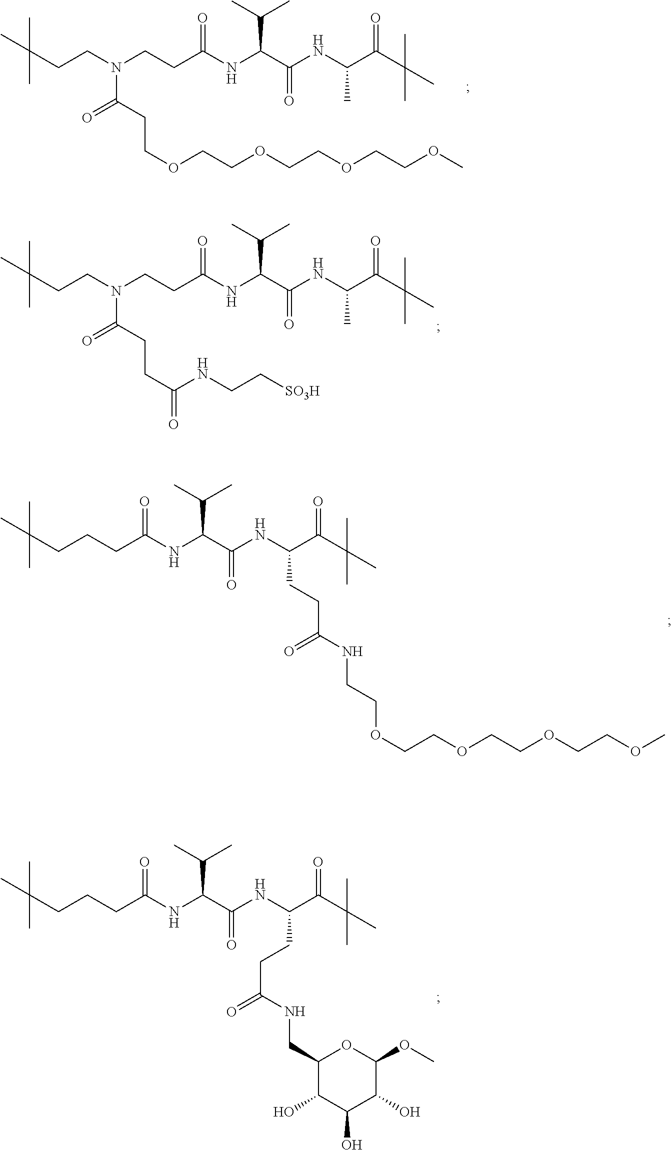

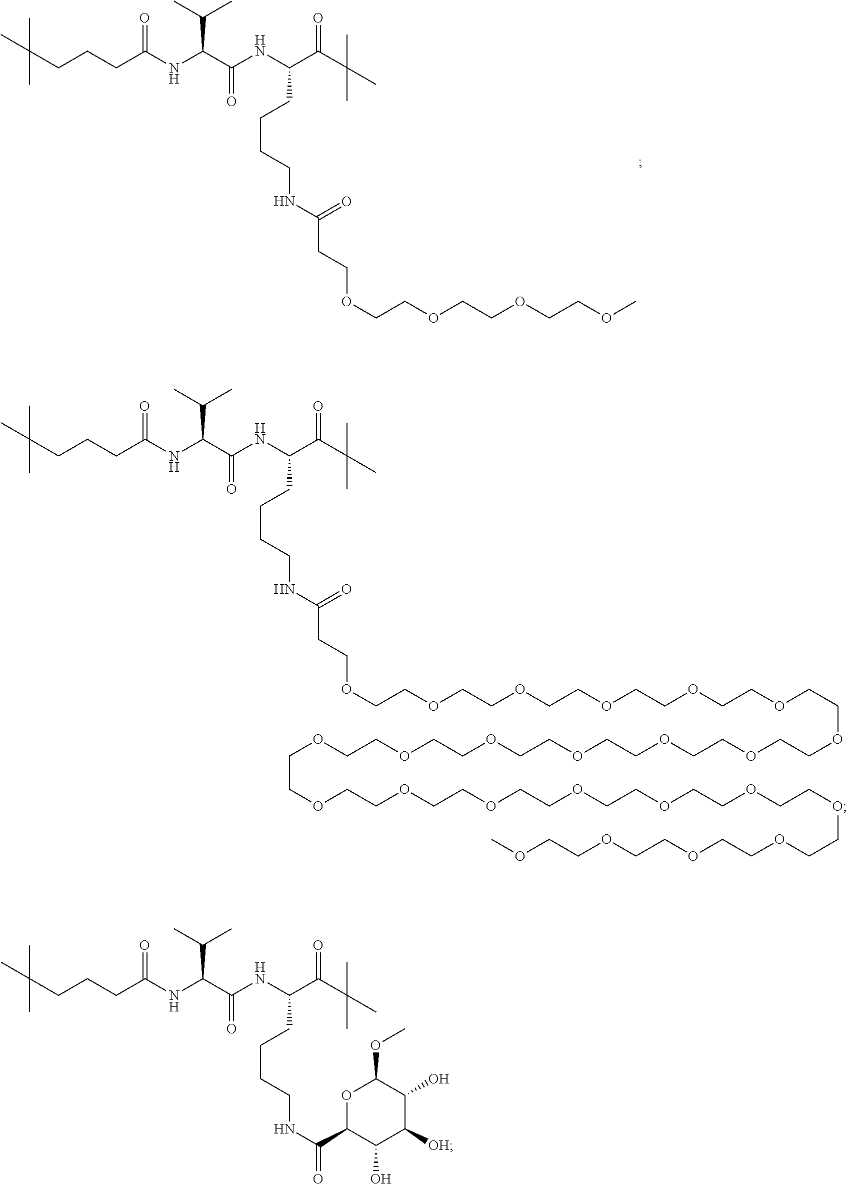

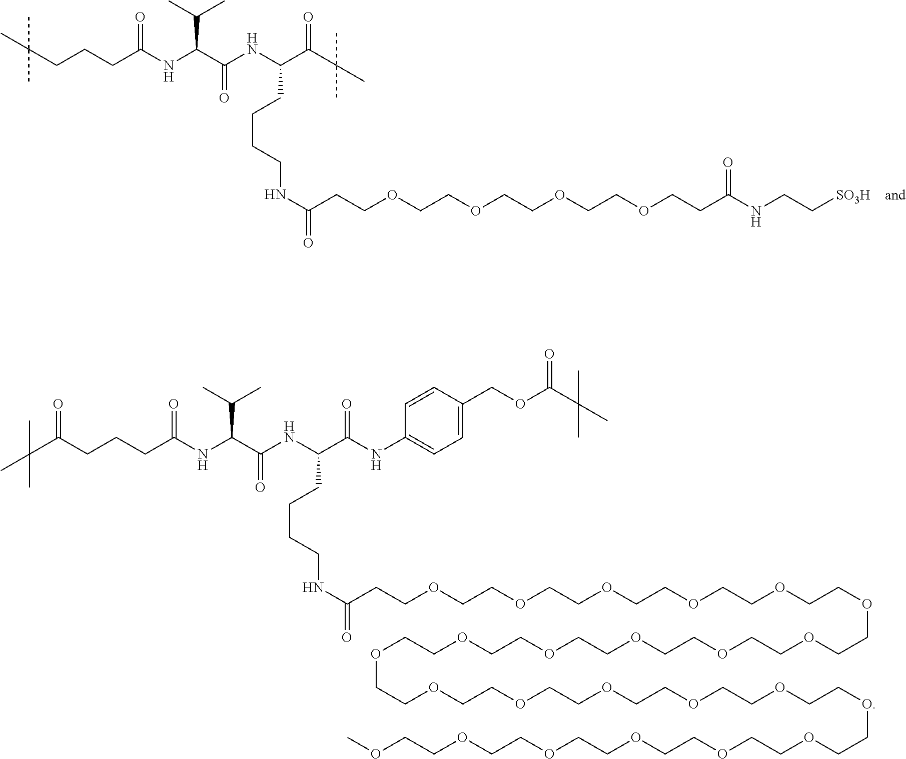

R₁ represents a (C₁-C₆)alkyl group, such as a methyl group;
R₂ and R₃ represent independently of each other a hydrogen atom or a (C₁-C₆)alkyl group such as a methyl group;
R₄ and R₅ represent independently of each other a (C₁-C₆)alkyl group, such as a methyl group;
R₇ and R₈ represent independently of each other a hydrogen atom or a (C₁-C₆)alkyl group, such as an isobutyl group or a neopentyl group, for instance one of R₇ and R₈ represents a (C₁-C₆)alkyl group, such as an isobutyl group or a neopentyl group and the other of R₇ and R₈ represents a hydrogen atom;
X represents an oxygen atom or NH;
R₉ represents two substituents selected from a (C₁-C₄)alkoxy group, such as a methoxy group, and a halogen atom, such as a chlorine atom, for instance R₉ represents 3-Cl and 4-methoxy; and
R₁₀ represents a hydrogen atom.

Among the compounds of formula (V) that are subject matter of the disclosure, a group of compounds is composed of the compounds of the following structure (beta epoxide configuration):

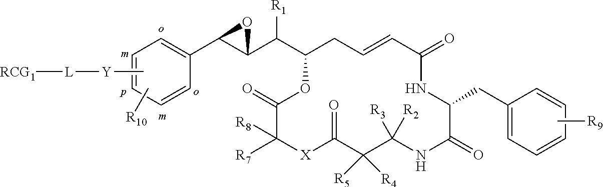

wherein:
R₁, R₂, R₃, R₄, R₅, R₆, R₇, R₈, R₉, R₁₀, R₁₁, R₁₂, n, RCG₁, X, Y and L are as defined in formula (IV).

All these sub-groups taken alone or in combination are part of the present disclosure.

Among the compounds of formula (V) that are the subject matter of the disclosure, mention may be made of the following compounds:

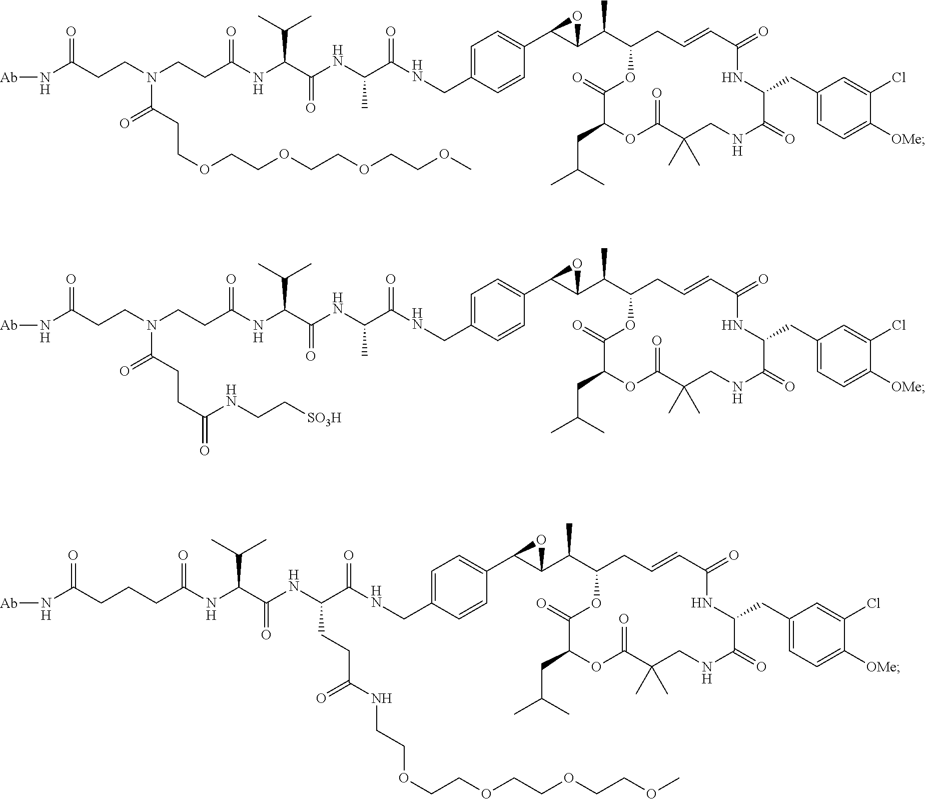

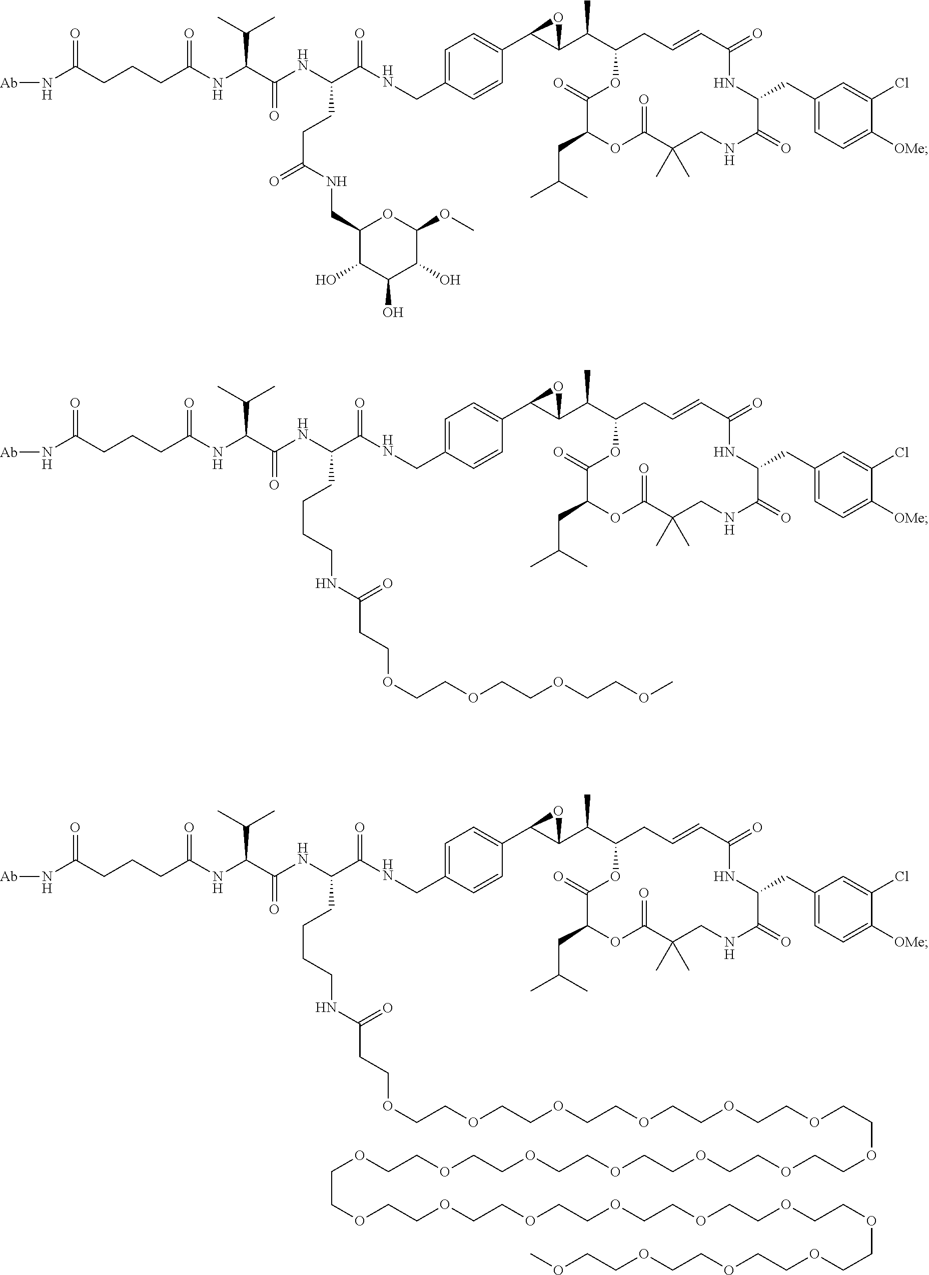

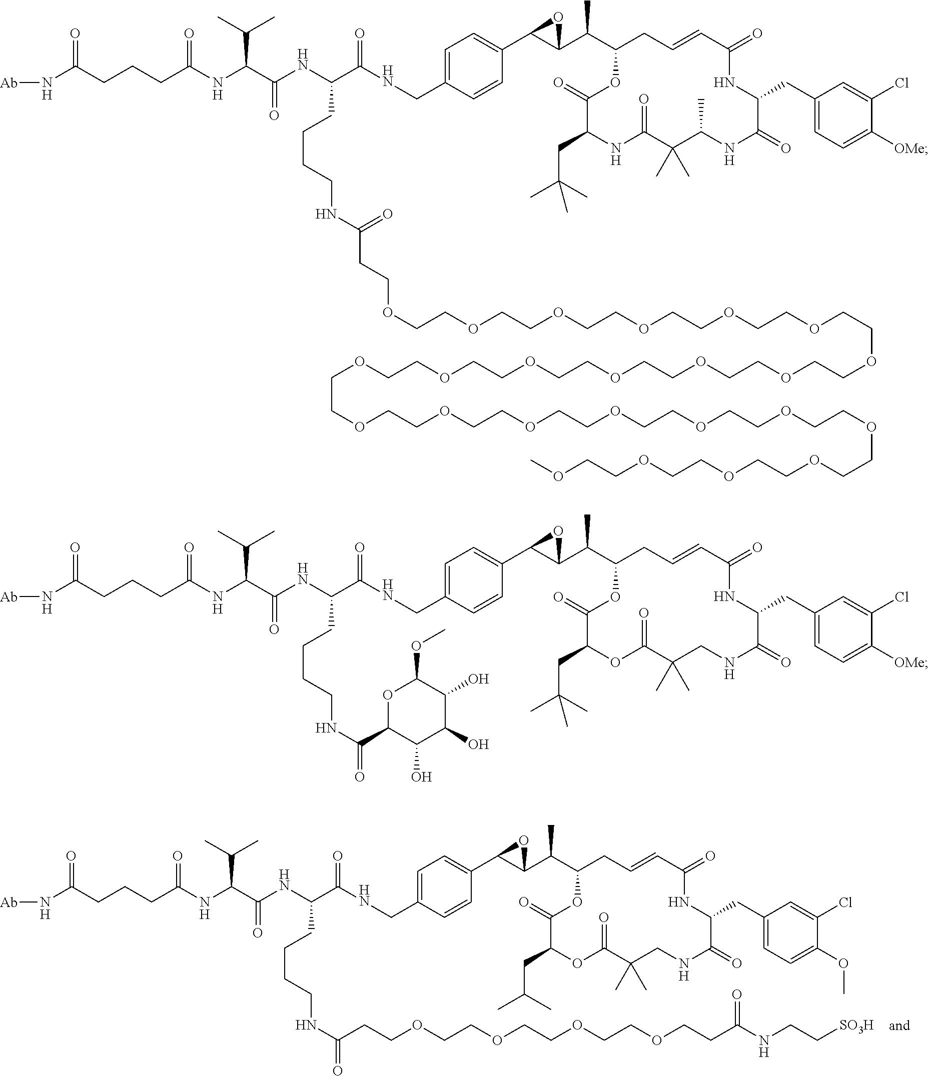

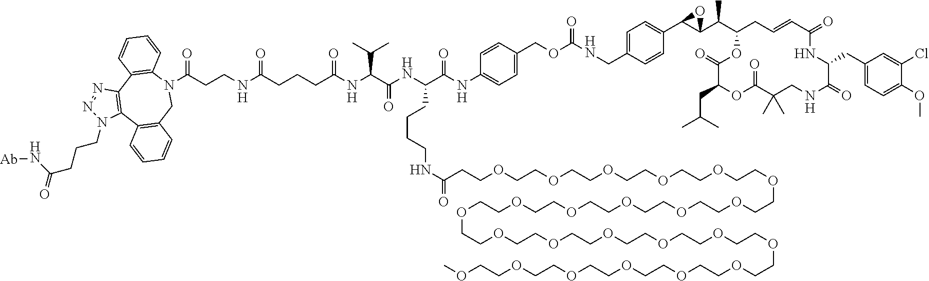

wherein Ab is as defined in formula (V) of the present disclosure.

In accordance with the disclosure, the compounds of general formula (I), (II), (III), (IV) and (V) can be prepared by the following processes.

### Preparation of the Linkers of Formula (II)

Preparation of the Spacers with Improved Hydrophilicity

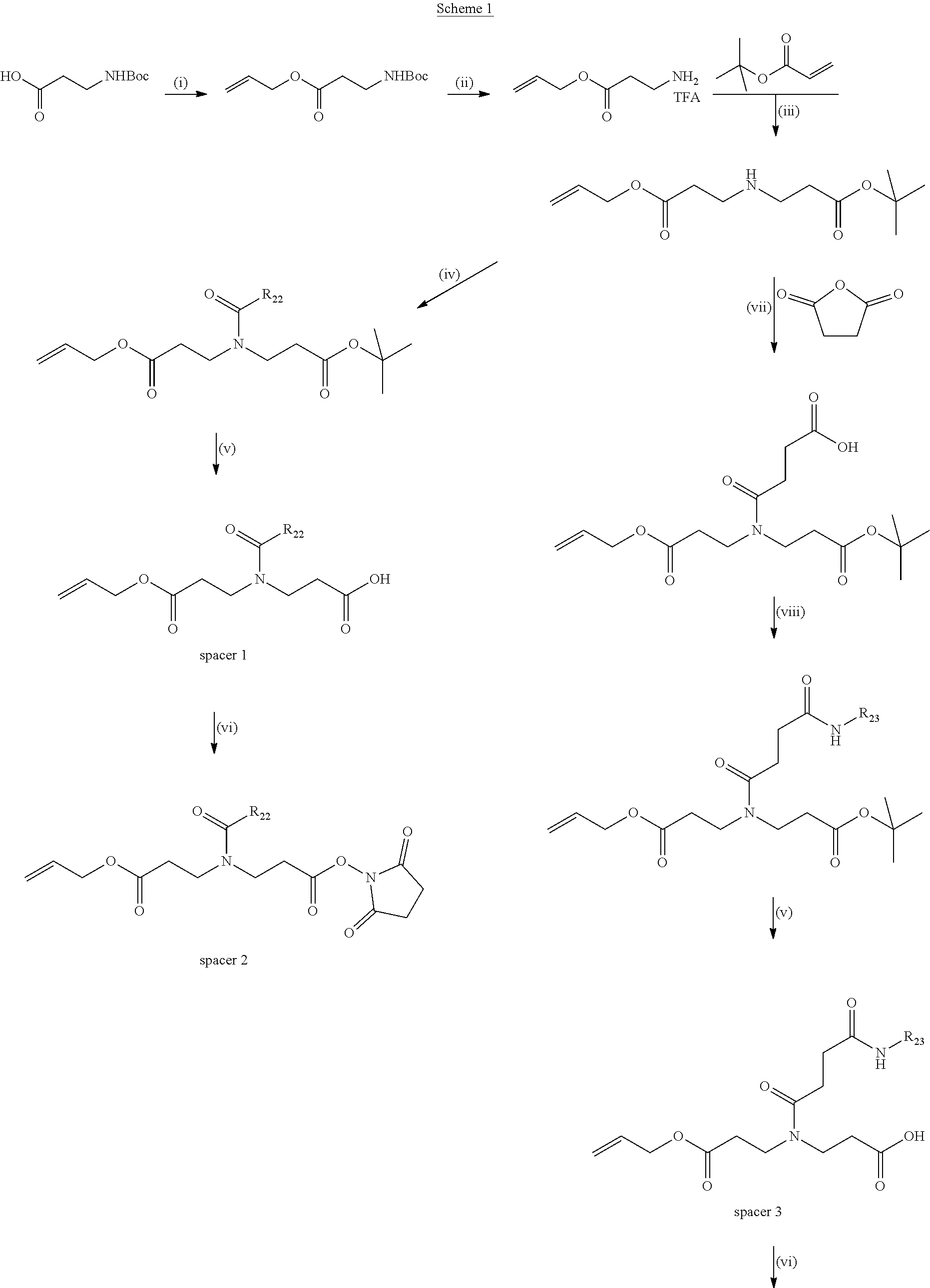

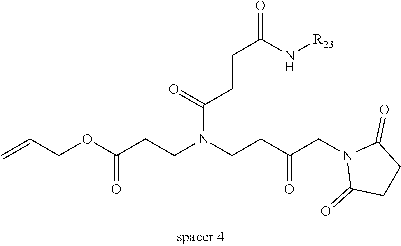

Step (i): protection of the carboxylic acid as an allyl ester using allyl bromide and a base such as, for example, cesium carbonate;
Step (ii): deprotection of the Boc amine using a solution of HCl (for example solution in dioxane) or of TFA;
Step (iii): addition of the amine on tert-butyl acrylate in the presence of a base such as, for example, DIEA;
Step (iv): coupling between the amine and an activated carboxylic acid such as a NHS ester or an acyl chloride in the presence of a base such as, for example, DIEA;
Step (v): deprotection of the tert-butyl ester using a solution of HCl (for example solution in dioxane) or of TFA;
Step (vi): activation of the carboxylic acid as a NHS ester by treatment with DSC in the presence of a base such as, for example, DIEA;
Step (vii): coupling to succinic anhydride;
Step (viii): activation of the carboxylic acid as a NHS ester by treatment with DSC in the presence of a base such as, for example, DIEA and coupling with an amine.

Scheme 1 depicted the synthesis starting with Boc-β-Ala-OH (CAS number [3303-84-2]) but may also apply to other Boc protected amino-alkyl acids which are commercially available for n ranging from 3 to 10. It depicted the synthesis using succinic anhydride but may also apply to glutaric anhydre or alkyl diacids which are commercially available for n ranging from 3 to 10.

Combination of the Spacers with Improved Hydrophilicity and the Classical Dipeptides

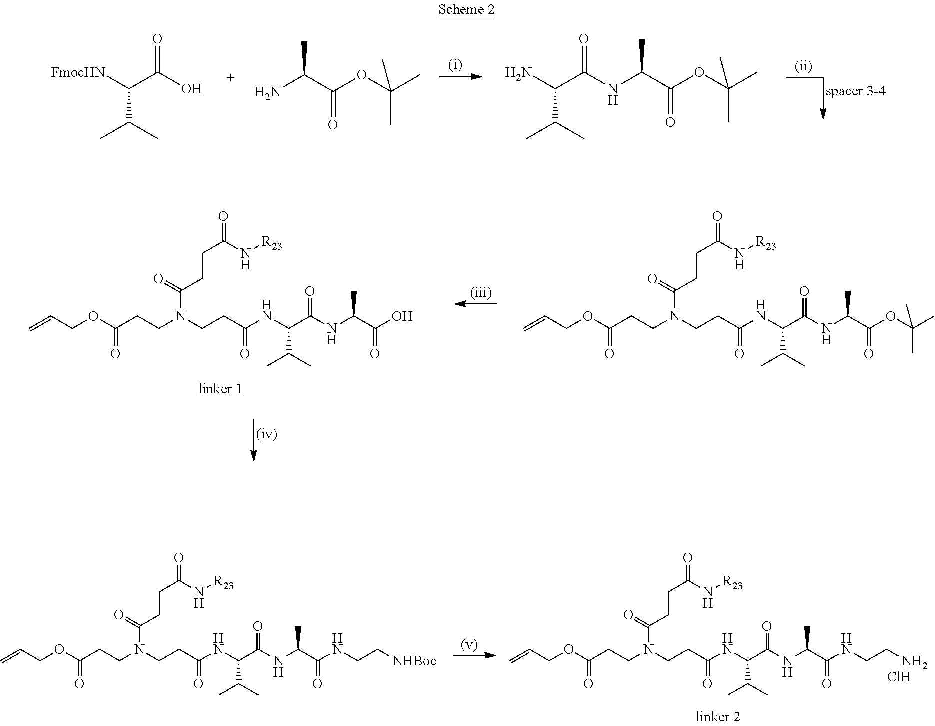

Step (i): peptidic coupling using coupling reagents such as, for example, EDC and HOBt and deprotection of the Fmoc amine in the presence of a base such as, for example, piperidine;
Step (ii): for spacer 3, activation of the carboxylic acid as a NHS ester by treatment with DSC in the presence of a base such as, for example, DIEA and coupling of spacers 3 and 4 with the dipeptide;
Step (iii): deprotection of the tert-butyl ester using a solution of HCl (for example solution in dioxane) or of TFA;
Step (iv): activation of the carboxylic acid as a NHS ester by treatment with DSC in the presence of a base such as, for example, DIEA and coupling with the amine;
Step (v): deprotection of the Boc amine using a solution of HCl (for example solution in dioxane) or of TFA.

Scheme 2 depicted the synthesis of linkers using a Val-Ala dipeptide but may also apply to other dipeptides; it depicted the synthesis using spacers 3 or 4 but may also apply to spacers 1 or 2; it depicted the synthesis using Boc-monoprotected ethylenediamine but may also apply to other Boc-monoprotected diamines which are commercially available for n ranging from 3 to 10.

Preparation of the Dipeptides with Improved Hydrophilicity
Preparation of the Dipeptides with Improved Hydrophilicity Based on Amino Acids Bearing a Carboxylic Acid Group on their Side Chain

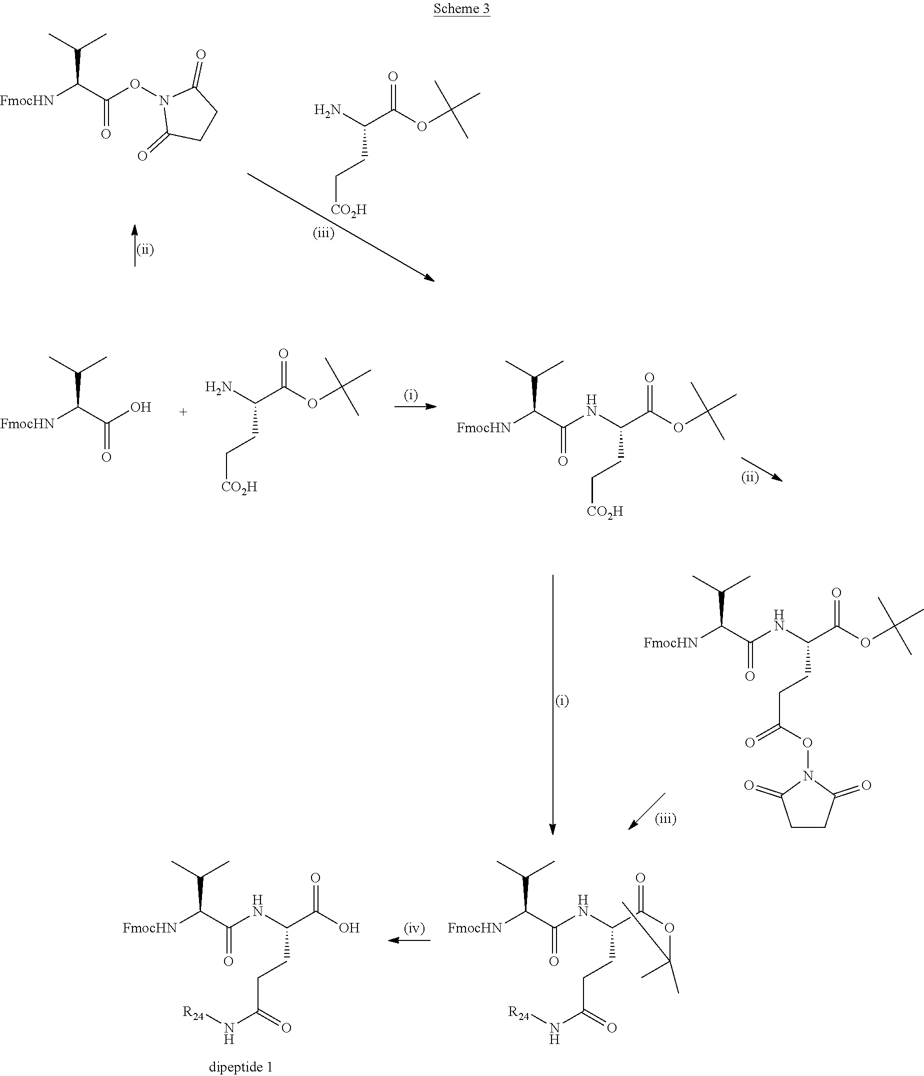

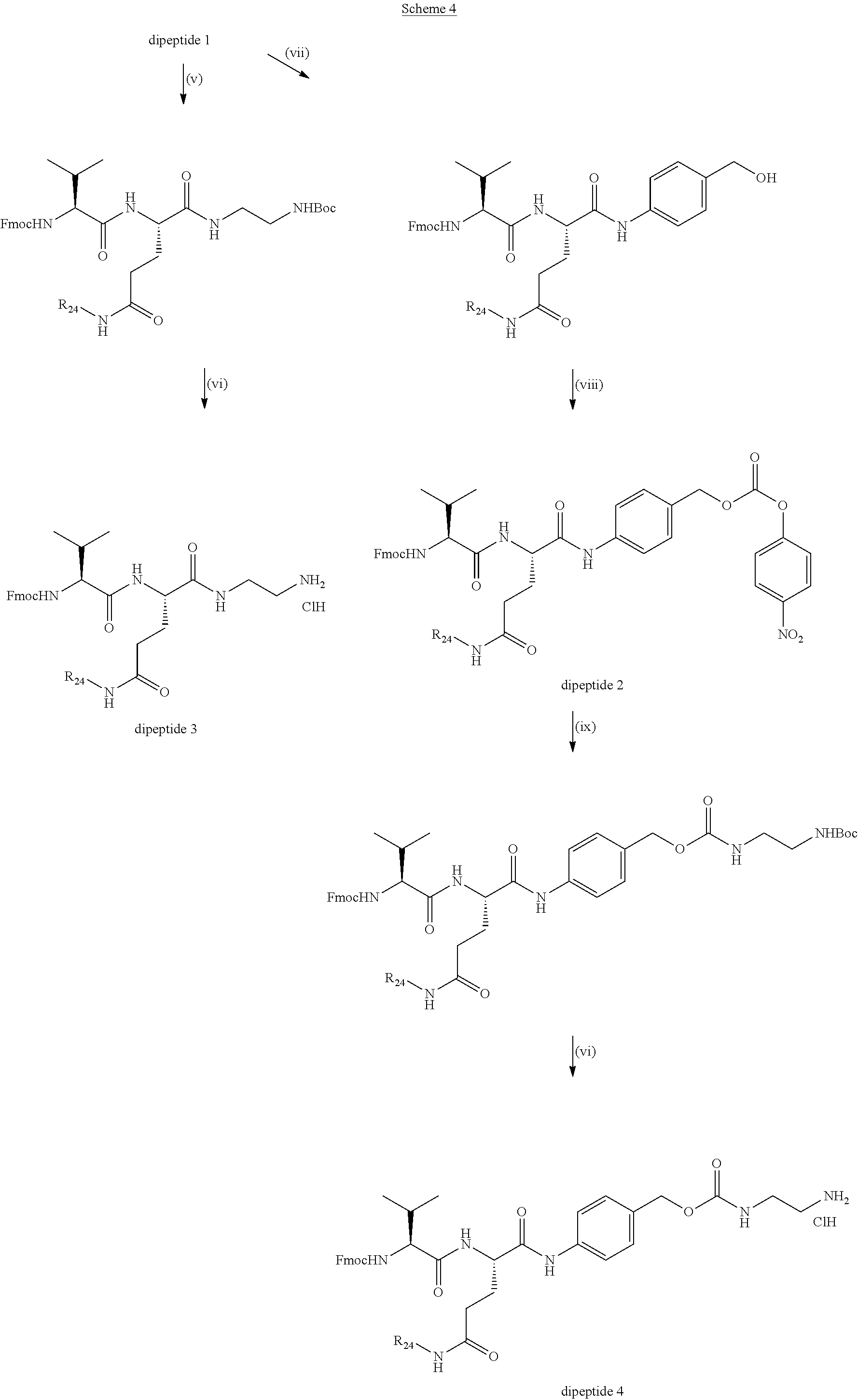

Step (i): peptidic coupling between the carboxylic acid and an amine using coupling reagents such as, for example, EDC and HOBt;
Step (ii): activation of the carboxylic acid as a NHS ester by treatment with DSC in the presence of a base such as, for example, DIEA;
Step (iii): peptidic coupling between the NHS ester and an amine in the presence of a base such as, for example, sodium bicarbonate or DIEA;
Step (iv): deprotection of the tert-butyl ester using a solution of HCl (for example solution in dioxane) or of TFA;
Step (v): peptidic coupling of the dipeptide with Boc-monoprotected ethylene diamine using a coupling reagent such as for example, T3P;
Step (vi): deprotection of the Boc amine using a solution of HCl (for example solution in dioxane) or of TFA;
Step (vii): peptidic coupling of the dipeptide with p-aminobenzyl alcohol using coupling reagents such as, for example, EEDQ;
Step (viii): activation of the benzylic alcohol as a p-nitrophenyl carbonate by treatment with p-nitrophenyl-chloroformate in the presence of a base such as, for example, DIEA;
Step (ix): formation of a carbamate between the activated alcohol and the amine in the presence of a base such as, for example, DIEA.

Scheme 3 depicted the synthesis of dipeptides starting with Val but may apply to other amino acids listed above; it depicted the synthesis using L-Glu tert-butyl ester (CAS number [45120-30-7]) but may also apply to other amino acids bearing a carboxylic acid on their side chain such as, for example, L-Asp tert-butyl ester (CAS number [4125-93-3]), D-Asp tert-butyl ester (CAS number [148823-36-3]), D-Glu tert-butyl ester (CAS number [25456-76-2]), 2-amino-hexanedioic acid 1-tert-butyl ester (CAS number [1245806-58-9]) or 2-amino-heptanedioic acid 1-tert-butyl ester (CAS number [1888498-03-0]). Scheme 4 depicted the synthesis of dipeptides using p-aminobenzyl alcohol (CAS number [623-04-1]) but may also apply to other aminobenzyl alcohol compounds which are commercially available such as, for example, 4-(1-hydroxyethyl)-aniline (racemic (CAS number [14572-89-5]) or enantiopure (R) (CAS number [210754-25-9]) or (S) (CAS number [500229-84-5])), 4-amino-α,α-dimethyl-benzene-methanol (CAS number [23243-04-1]), 4-amino-α-methoxy-α-methyl-benzenemethanol (CAS number [1379318-81-6]), 4-amino-α-methyl-α-trifluoromethyl-benzenemethanol (CAS number [851652-56-7]), 2-amino-benzenemethanol (CAS number [5344-90-1]), 2-amino-α-methyl-benzenemethanol (racemic (CAS number [10517-50-7]) or enantiopure (R) (CAS number [3205-21-8]) or (S) (CAS number [3205-21-8])), 6-amino-3-pyridinemethanol (CAS number [113293-71-3]), 6-amino-α-methyl-3-pyridinemethanol (CAS number [1335054-83-5]), 6-amino-α-ethyl-3-pyridinemethanol (CAS number [1355225-85-2]), 6-amino-α,α-dimethyl-3-pyridinemethanol (CAS number [843646-03-8]), 5-amino-3-pyridinemethanol (CAS number [873651-92-4]), 2-amino-3-pyridinemethanol (CAS number [23612-57-9]), 2-amino-α-methyl-3-pyridinemethanol (racemic (CAS number [869567-91-9]) or enantiopure (R) (CAS number [936718-01-3]) or (S) (CAS number [936718-00-2])), 2-amino-α-ethyl-3-pyridinemethanol (CAS number [914223-90-8]), 2-amino-α,α-dimethyl-3-pyridinemethanol (CAS number [213666-96-7]), 3-amino-4-pyridinemethanol (CAS number [152398-05-5]), 3-amino-α-methyl-4-pyridinemethanol (CAS number [1242470-88-7]), 3-amino-α,α-methyl-4-pyridinemethanol (CAS number [13357-81-8]), 4-amino-3-pyridinemethanol (CAS number [138116-34-4]), 4-amino-α-methyl-3-pyridinemethanol (CAS number [741223-49-4]), 4-amino-α,α-methyl-3-pyridinemethanol (CAS number [1339013-26-1]), 3-amino-2-pyridinemethanol (CAS number [52378-63-9]), 3-amino-α-methyl-2-pyridinemethanol (CAS number [954240-54-1]), 3-amino-α,α-methyl-2-pyridinemethanol (CAS number [899438-57-4]); it depicted the synthesis using Boc-monoprotected ethylenediamine but may also apply to other Boc-monoprotected diamines which are commercially available for n ranging from 3 to 10.

Preparation of the Dipeptides with Improved Hydrophilicity Based on Amino Acids Bearing an Amino Group on their Side Chain

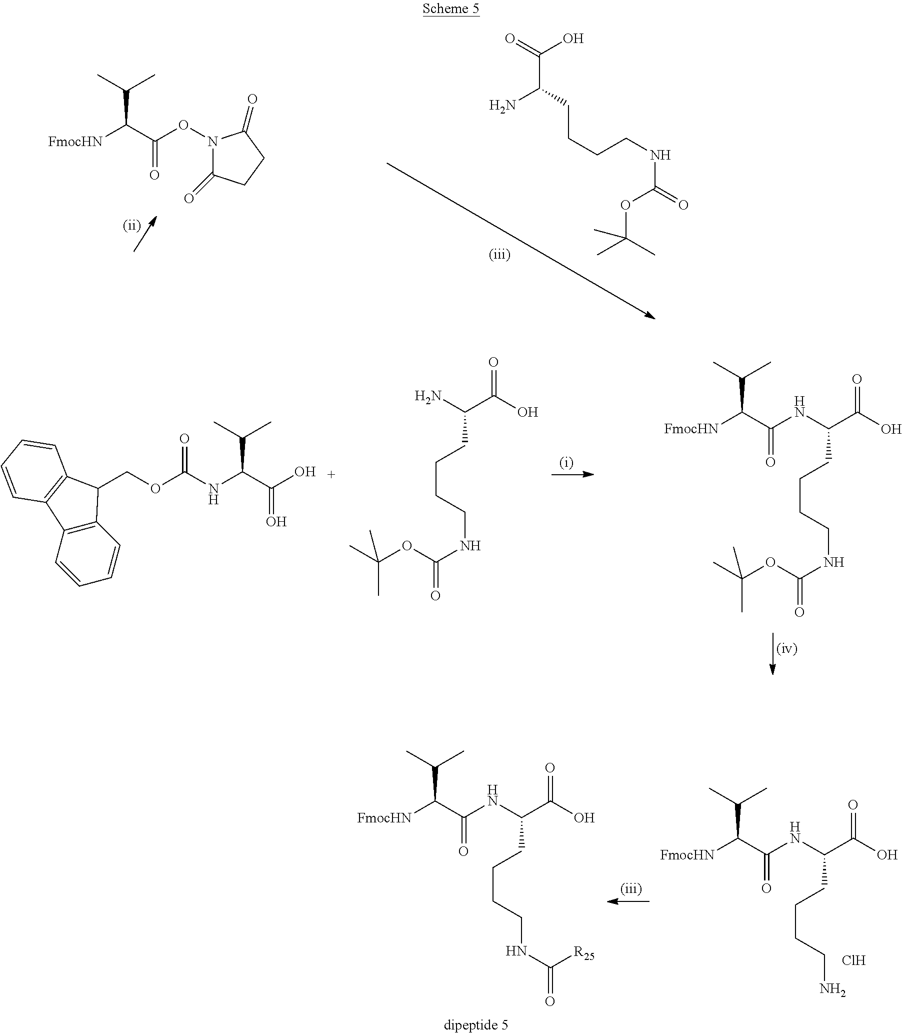

Or alternatively dipeptide 5 can be prepared following the synthesis described in Scheme 6.

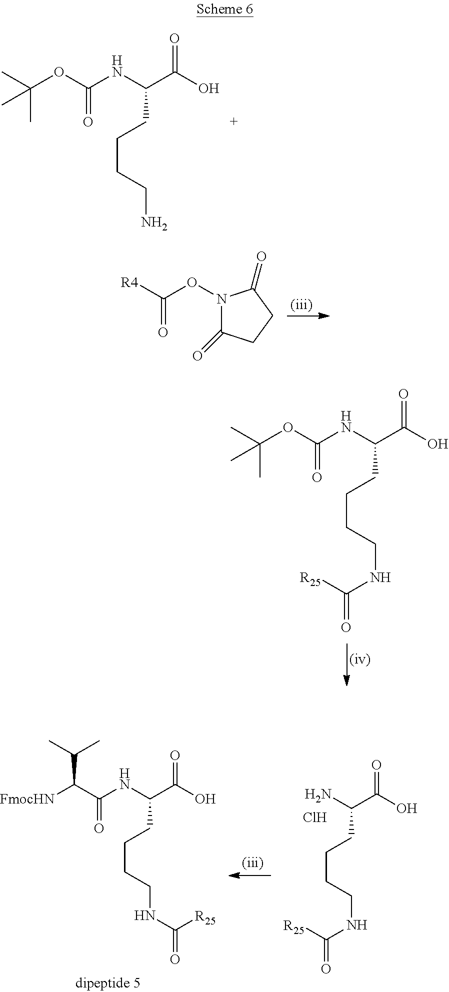

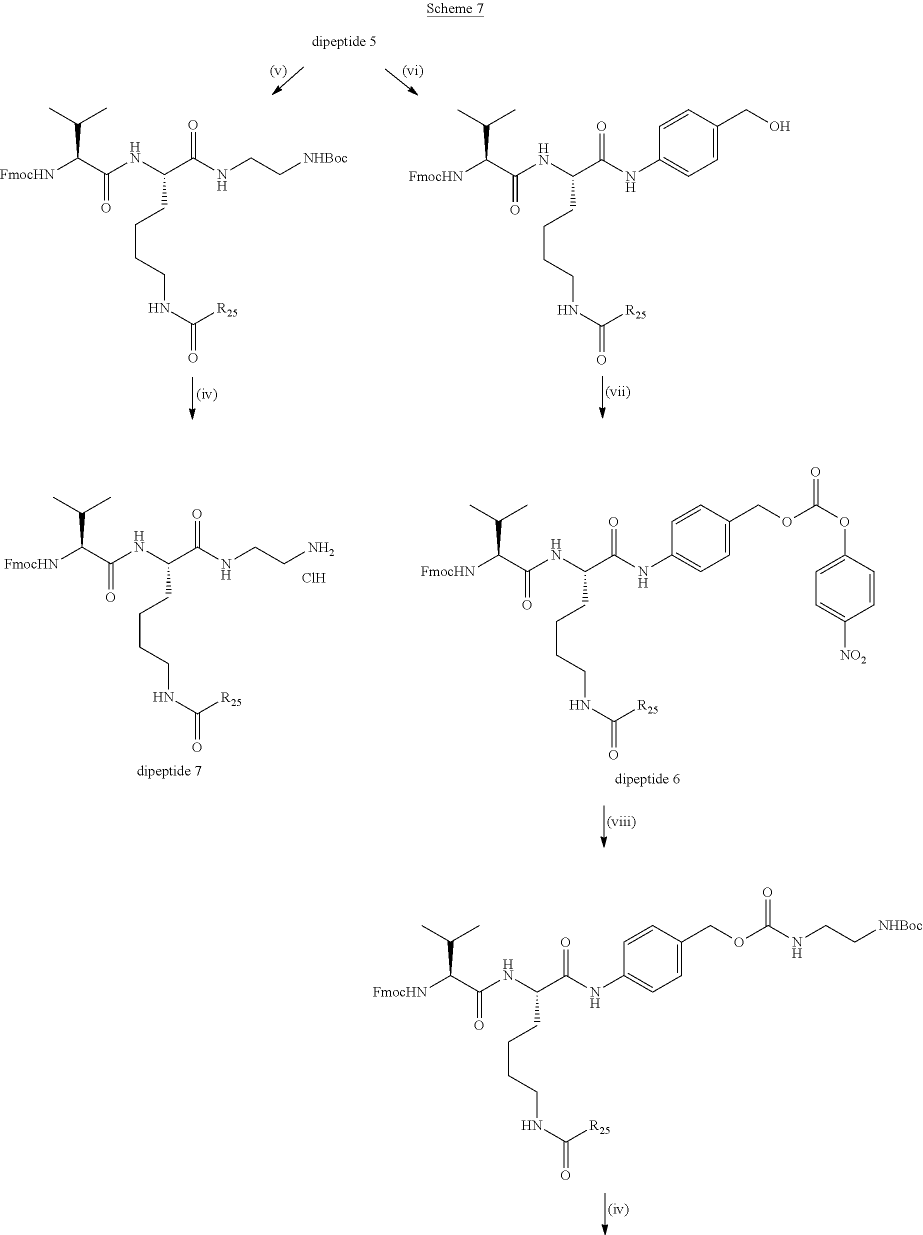

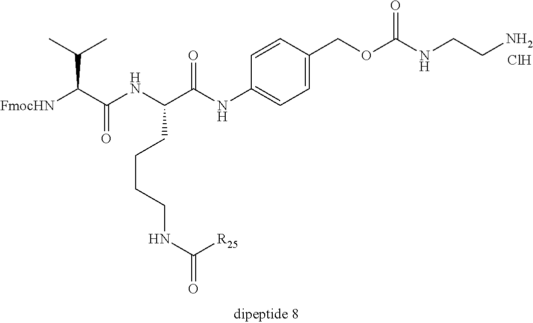

Step (i): peptidic coupling between the carboxylic acid and an amine using coupling reagents such as, for example, EDC and HOBt;
Step (ii): activation of the carboxylic acid as a NHS ester by treatment with DSC in the presence of a base such as, for example, DIEA;
Step (iii): peptidic coupling between the NHS ester and an amine in the presence of a base such as, for example, sodium bicarbonate or DIEA;
Step (iv): deprotection of the Boc amine using a solution of HCl (for example solution in dioxane) or of TFA;
Step (v): peptidic coupling of the dipeptide with Boc-monoprotected ethylene diamine using a coupling reagent such as for example, T3P;
Step (vi): peptidic coupling of the dipeptide with p-aminobenzyl alcohol using coupling reagents such as, for example, EEDQ;
Step (vii): activation of the benzylic alcohol as a p-nitrophenyl carbonate by treatment with p-nitrophenyl-chloroformate in the presence of a base such as, for example, DIEA;
Step (viii): formation of a carbamate between the activated alcohol and the amine in the presence of a base such as, for example, DIEA.

Schemes 5 and 6 depicted the synthesis of dipeptides starting with Val but may apply to other amino acids listed above. Scheme 5 depicted the synthesis using L-Lys Boc-protected on the side chain (CAS number [2418-95-3]) but may also apply to other amino acids bearing an amino group on their side chain such as, for example, (S)-3-amino-2-(tert-butoxycarbonylamino)propanoic acid (CAS number [73259-81-1]), (S)-3-amino-2-(tert-butoxycarbonylamino)propanoic acid (CAS number [76387-70-7]), (S)-4-amino-2-(tert-butoxycarbonylamino)butanoic acid (CAS number [25691-37-6]), (R)-4-amino-2-(tert-butoxycarbonylamino)butanoic acid (CAS number [80445-78-9]), L-ornithine Boc-protected on the side chain (CAS number [21887-64-9]), D-ornithine Boc-protected on the side chain (CAS number [159877-12-0]), D-Lys Boc-protected on the side chain (CAS number [106719-44-2]), (2S)-2-amino-7-[[(1,1-dimethylethoxy)carbonyl]amino]heptanoic acid (CAS number [1142814-17-2]) or (2R)-2-amino-7-[[(1,1-dimethylethoxy)carbonyl]amino]heptanoic acid (CAS number [117833-90-6]). Scheme 7 depicted the synthesis of dipeptides using p-aminobenzyl alcohol (CAS number [623-04-1]) but may also apply to other aminobenzyl alcohol compounds which are commercially available such as, for example, 4-(1-hydroxyethyl)-aniline (racemic (CAS number [14572-89-5]) or enantiopure (R) (CAS number [210754-25-9]) or (S) (CAS number [500229-84-5])), 4-amino-α,α-dimethyl-benzene-methanol (CAS number [23243-04-1]), 4-amino-α-methoxy-α-methyl-benzenemethanol (CAS number [1379318-81-6]), 4-amino-α-methyl-α-trifluoromethyl-benzenemethanol (CAS number [851652-56-7]), 2-amino-benzenemethanol (CAS number [5344-90-1]), 2-amino-α-methyl-benzenemethanol (racemic (CAS number [10517-50-7]) or enantiopure (R) (CAS number [3205-21-8]) or (S) (CAS number [3205-21-8])), 6-amino-3-pyridinemethanol (CAS number [113293-71-3]), 6-amino-α-methyl-3-pyridinemethanol (CAS number [1335054-83-5]), 6-amino-α-ethyl-3-pyridinemethanol (CAS number [1355225-85-2]), 6-amino-α,α-dimethyl-3-pyridinemethanol (CAS number [843646-03-8]), 5-amino-3-pyridinemethanol (CAS number [873651-92-4]), 2-amino-3-pyridinemethanol (CAS number [23612-57-9]), 2-amino-α-methyl-3-pyridinemethanol (racemic (CAS number [869567-91-9]) or enantiopure (R) (CAS number [936718-01-3]) or (S) (CAS number [936718-00-2])), 2-amino-α-ethyl-3-pyridinemethanol (CAS number [914223-90-8]), 2-amino-α,α-dimethyl-3-pyridinemethanol (CAS number [213666-96-7]), 3-amino-4-pyridinemethanol (CAS number [152398-05-5]), 3-amino-α-methyl-4-pyridinemethanol (CAS number [1242470-88-7]), 3-amino-α,α-methyl-4-pyridinemethanol (CAS number [13357-81-8]), 4-amino-3-pyridinemethanol (CAS number [138116-34-4]), 4-amino-α-methyl-3-pyridinemethanol (CAS number [741223-49-4]), 4-amino-α,α-methyl-3-pyridinemethanol (CAS number [1339013-26-1]), 3-amino-2-pyridinemethanol (CAS number [52378-63-9]), 3-amino-α-methyl-2-pyridinemethanol (CAS number [954240-54-1]), 3-amino-α,α-methyl-2-pyridinemethanol (CAS number [899438-57-4]); it depicted the synthesis using Boc-monoprotected ethylenediamine but may also apply to other Boc-monoprotected diamines which are commercially available for n ranging from 3 to 10.

### Preparation of the Cryptophycin Compounds

Cryptophycin compounds may be prepared as described in WO2011/001052 for X═O and in PCT/EP2016/076603 for X=NH.

### Preparation of the New Cryptophycin Payloads of Formula (IV) for Y=NH—CH₂

Preparation of the New Cryptophycin Payloads Bearing a Spacer with Improved Hydrophilicity

### Without PABA Moiety

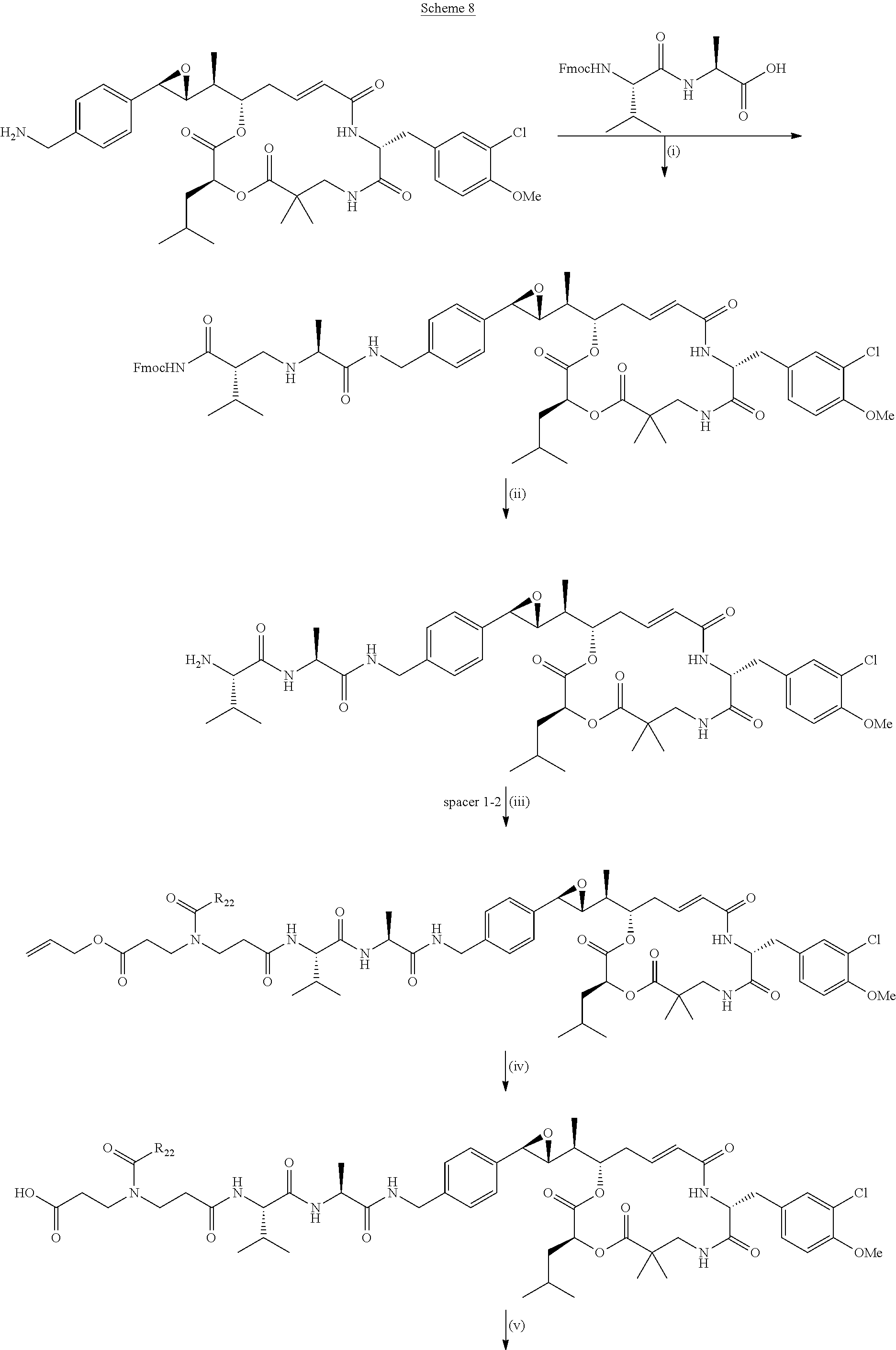

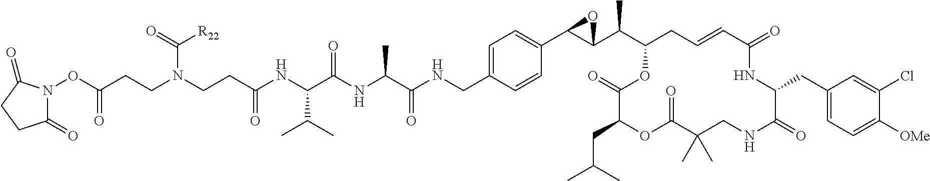

Step (i): peptidic coupling between the cryptophycin derivative and the dipeptide using coupling reagents such as, for example, EDC and HOBt;
Step (ii): deprotection of the Fmoc amine in the presence of a base such as, for example, piperidine;
Step (iii): peptidic coupling with spacer 1 using coupling reagents such as, for example, EDC and HOBt, or coupling with spacer 2 in the presence of a base such as, for example, DIEA;
Step (iv): deprotection of the allyl ester in the presence of a catalyst such as, for example, tetrakis-(triphenylphosphine)palladium;
Step (v): activation of the carboxylic acid as a NHS ester by treatment with DSC in the presence of a base such as, for example, DIEA.

Alternatively, new cryptophycin payloads bearing a spacer with improved hydrophilicity may also be prepared as depicted in Scheme 9.

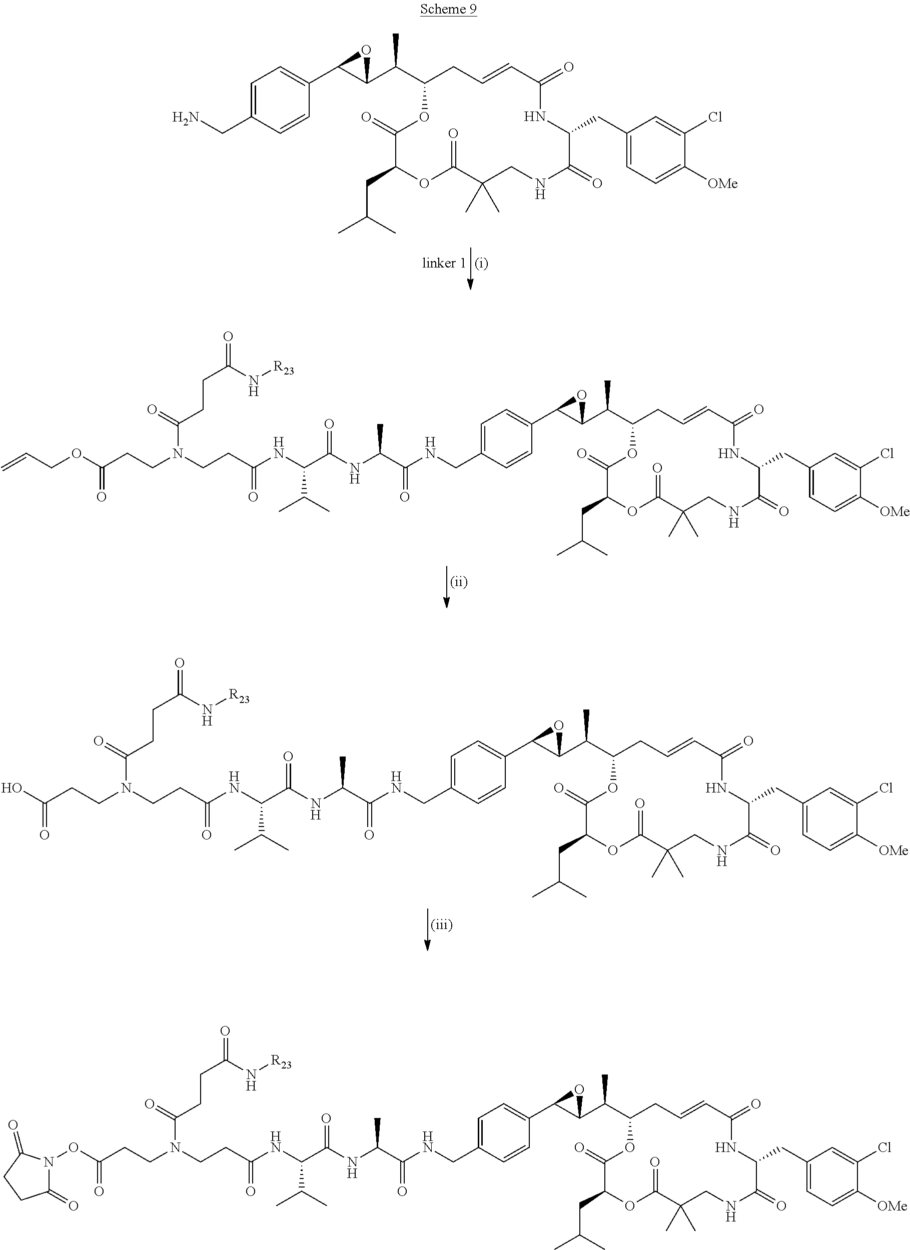

Step (i): activation of the carboxylic acid of linker 1 as a NHS ester by treatment with DSC in the presence of a base such as, for example, DIEA and coupling with the cryptophycin amine.
Step (ii): deprotection of the allyl ester in the presence of a catalyst such as, for example, tetrakis-(triphenylphosphine)palladium;
Step (iii): activation of the carboxylic acid as a NHS ester by treatment with DSC in the presence of a base such as, for example, DIEA.

Schemes 8 and 9 depicted the synthesis of payloads using the p-benzylic amine of C52 but may also apply to other cryptophycin compounds; it depicted the synthesis using Val-Ala dipeptides but may also apply to other dipeptides; it depicted the synthesis using spacers 1 or 2 but may also apply to spacers 3 or 4.

### With PABA Moeity

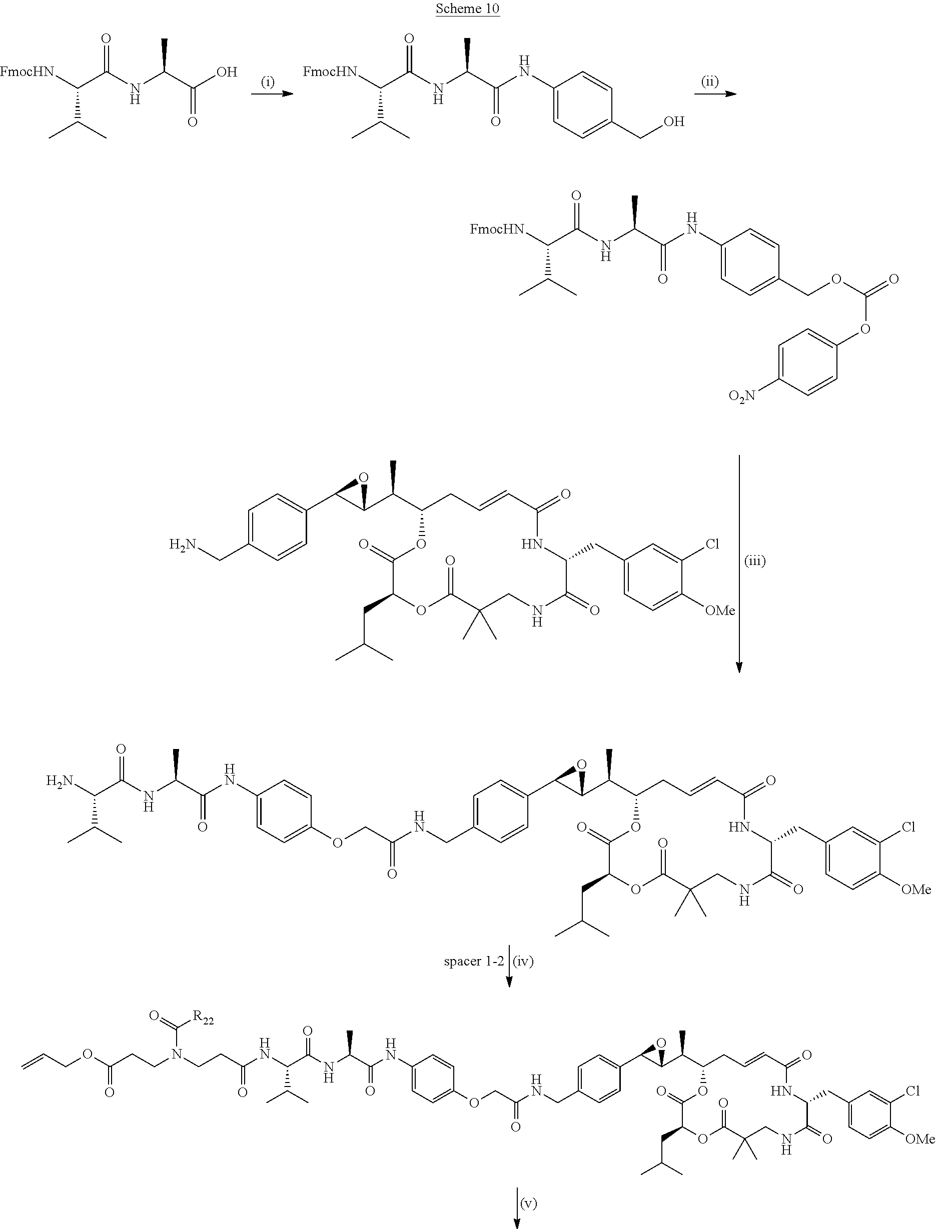

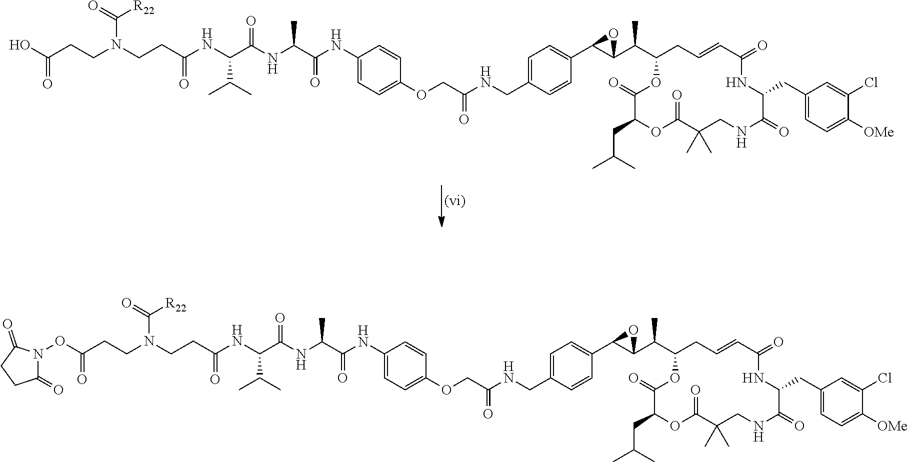

Step (i): peptidic coupling between Fmoc-Val-Ala-OH and 4-aminobenzyl alcohol in the presence of a coupling reagent such as, for example, EEDQ;
Step (ii): activation of the benzylic alcohol as a p-nitrophenyl carbonate by treatment with p-nitrophenyl-chloroformate in the presence of a base such as, for example, DIEA;
Step (iii): formation of a carbamate between the activated alcohol and the cryptophycin amine in the presence of a base such as, for example, DIEA and deprotection of the Fmoc amine in the presence of a base such as, for example, piperidine;
Step (iv): peptidic coupling with spacer 1 using coupling reagents such as, for example, EDC and HOBt, or coupling with spacer 2 in the presence of a base such as, for example, DIEA;
Step (v): deprotection of the allyl ester in the presence of a catalyst such as, for example, tetrakis-(triphenylphosphine)palladium;
Step (vi): activation of the carboxylic acid as a NHS ester by treatment with DSC in the presence of a base such as, for example, DIEA.

Scheme 10 depicted the synthesis of payloads using Val-Ala dipeptides but may also apply to other dipeptides; it depicted the synthesis using p-aminobenzyl alcohol (CAS number [623-04-1]) but may also apply to other aminobenzyl alcohol compounds which are commercially available such as, for example, 4-(1-hydroxyethyl)-aniline (racemic (CAS number [14572-89-5]) or enantiopure (R) (CAS number [210754-25-9]) or (S) (CAS number [500229-84-5])), 4-amino-α,α-dimethyl-benzene-methanol (CAS number [23243-04-1]), 4-amino-α-methoxy-α-methyl-benzenemethanol (CAS number [1379318-81-6]), 4-amino-α-methyl-α-trifluoromethyl-benzenemethanol (CAS number 15 [851652-56-7]), 2-amino-benzenemethanol (CAS number [5344-90-1]), 2-amino-α-methyl-benzenemethanol (racemic (CAS number [10517-50-7]) or enantiopure (R) (CAS number [3205-21-8]) or (S) (CAS number [3205-21-8])), 6-amino-3-pyridinemethanol (CAS number [113293-71-3]), 6-amino-α-methyl-3-pyridinemethanol (CAS number [1335054-83-5]), 6-amino-α-ethyl-3-pyridinemethanol (CAS number [1355225-85-2]), 6-amino-α,α-dimethyl-3-pyridinemethanol (CAS number [843646-03-8]), 5-amino-3-pyridinemethanol (CAS number [873651-92-4]), 2-amino-3-pyridinemethanol (CAS number [23612-57-9]), 2-amino-α-methyl-3-pyridinemethanol (racemic (CAS number [869567-91-9]) or enantiopure (R) (CAS number [936718-01-3]) or (S) (CAS number [936718-00-2])), 2-amino-α-ethyl-3-pyridinemethanol (CAS number [914223-90-8]), 2-amino-α,α-dimethyl-3-pyridinemethanol (CAS number [213666-96-7]), 3-amino-4-pyridinemethanol (CAS number [152398-05-5]), 3-amino-α-methyl-4-pyridinemethanol (CAS number [1242470-88-7]), 3-amino-α,α-methyl-4-pyridinemethanol (CAS number [13357-81-8]), 4-amino-3-pyridinemethanol (CAS number [138116-34-4]), 4-amino-α-methyl-3-pyridinemethanol (CAS number [741223-49-4]), 4-amino-α,α-methyl-3-pyridinemethanol (CAS number [1339013-26-1]), 3-amino-2-pyridinemethanol (CAS number [52378-63-9]), 3-amino-α-methyl-2-pyridinemethanol (CAS number [954240-54-1]), 3-amino-α,α-methyl-2-pyridinemethanol (CAS number [899438-57-4]); it depicted the synthesis using the p-benzylic amine of C52 but may also apply to other cryptophycin compounds; it depicted the synthesis using spacers 1 or 2 but may also apply to spacers 3 or 4.

Preparation of the New Cryptophycin Payloads of Formula (IV) Bearing a Dipeptide with Improved Hydrophilicity
Without PABA Moiety

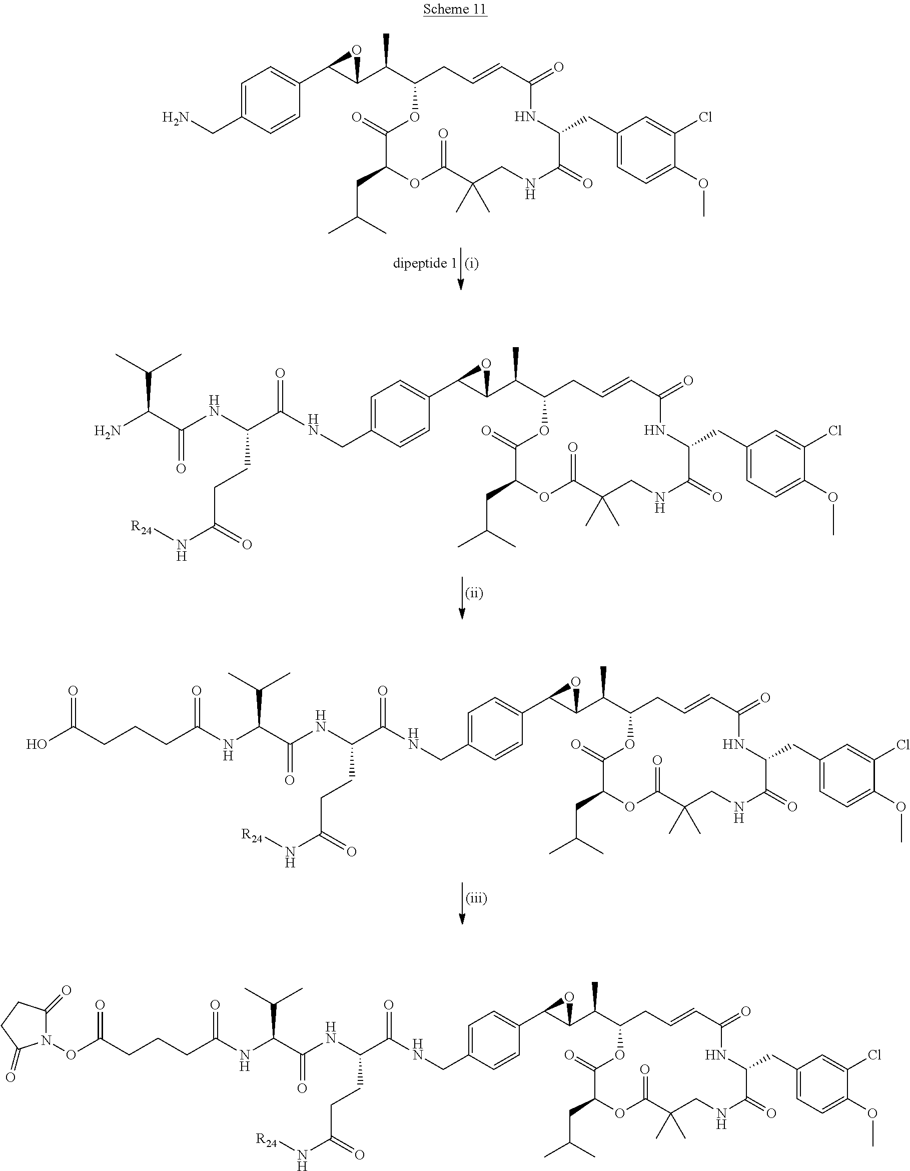

Step (i): peptidic coupling between the cryptophycin derivative and the dipeptide using coupling reagents such as, for example, EDC and HOBt and deprotection of the Fmoc amine in the presence of a base such as, for example, piperidine;
Step (ii): coupling to glutaric anhydride; one additional deprotection step of the side chain of substituted amino acid may be required prior to step (iii);
Step (iii): activation of the carboxylic acid as a NHS ester by treatment with DSC in the presence of a base such as, for example, DIEA.

Scheme 11 depicted the synthesis of payloads using the p-benzylic amine of C52 but may also apply to other cryptophycin compounds; it depicted the synthesis using dipeptide 1 but may also apply to dipeptide 5; it depicted the synthesis using glutaric anhydride but may also apply to succinic anhydride or alkyl diacids which are commercially available for n ranging from 3 to 10.

### With PABA Moiety

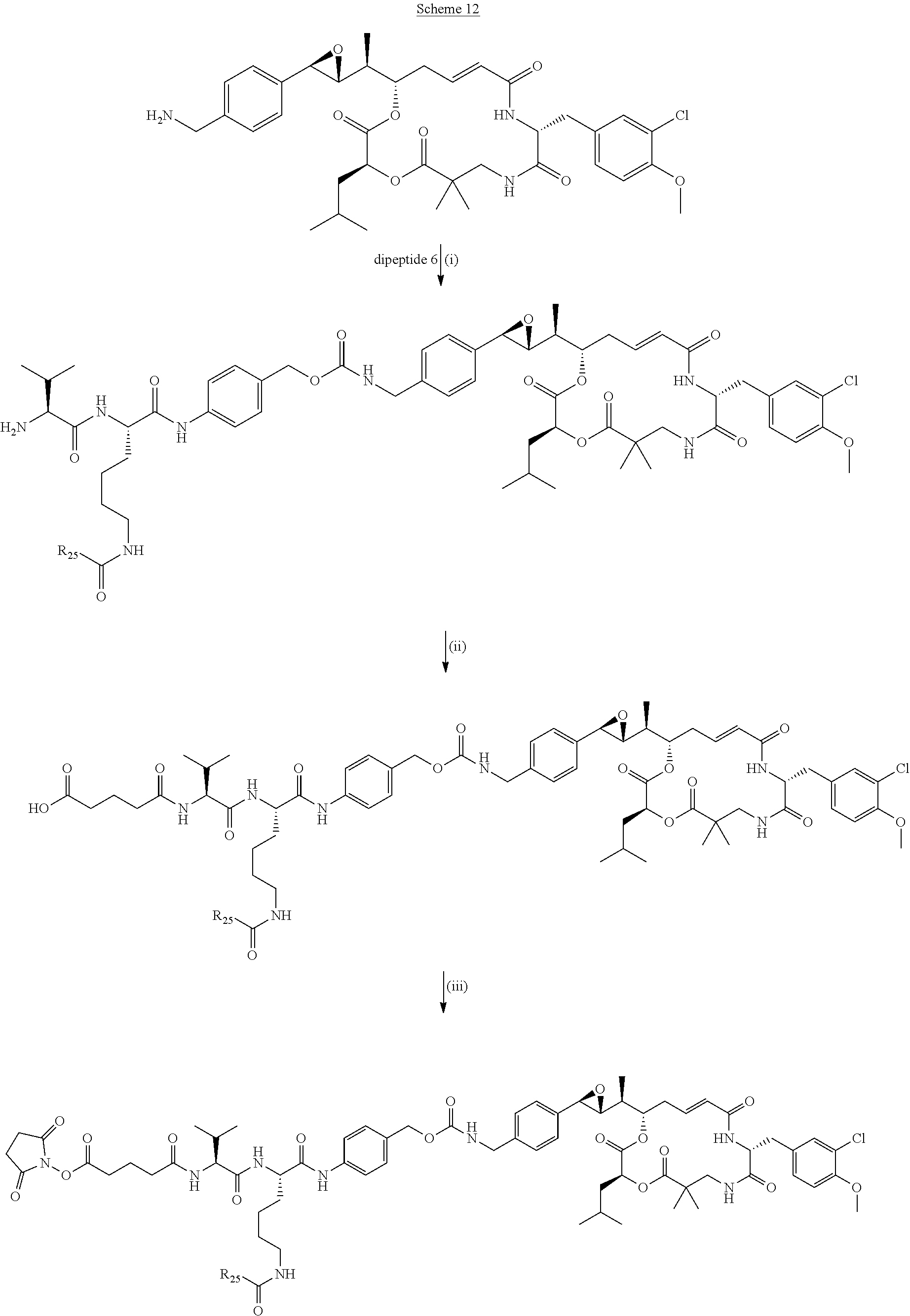

Step (i): formation of a carbamate between the activated alcohol and the cryptophycin amine in the presence of a base such as, for example, DIEA and deprotection of the Fmoc amine in the presence of a base such as, for example, piperidine;
Step (ii): coupling to glutaric anhydride; one additional deprotection step of the side chain of substituted amino acid may be required prior to step (iii);
Step (iii): activation of the carboxylic acid as a NHS ester by treatment with DSC in the presence of a base such as, for example, DIEA.

Scheme 12 depicted the synthesis of payloads using the p-benzylic amine of C52 but may also apply to other cryptophycin compounds; it depicted the synthesis using dipeptide 6 but may also apply to dipeptide 2.

Preparation of the New Cryptophycin Payloads of Formula (IV) Bearing Both a Spacer and a Dipeptide with Improved Hydrophilicity

### Without PABA Moeity

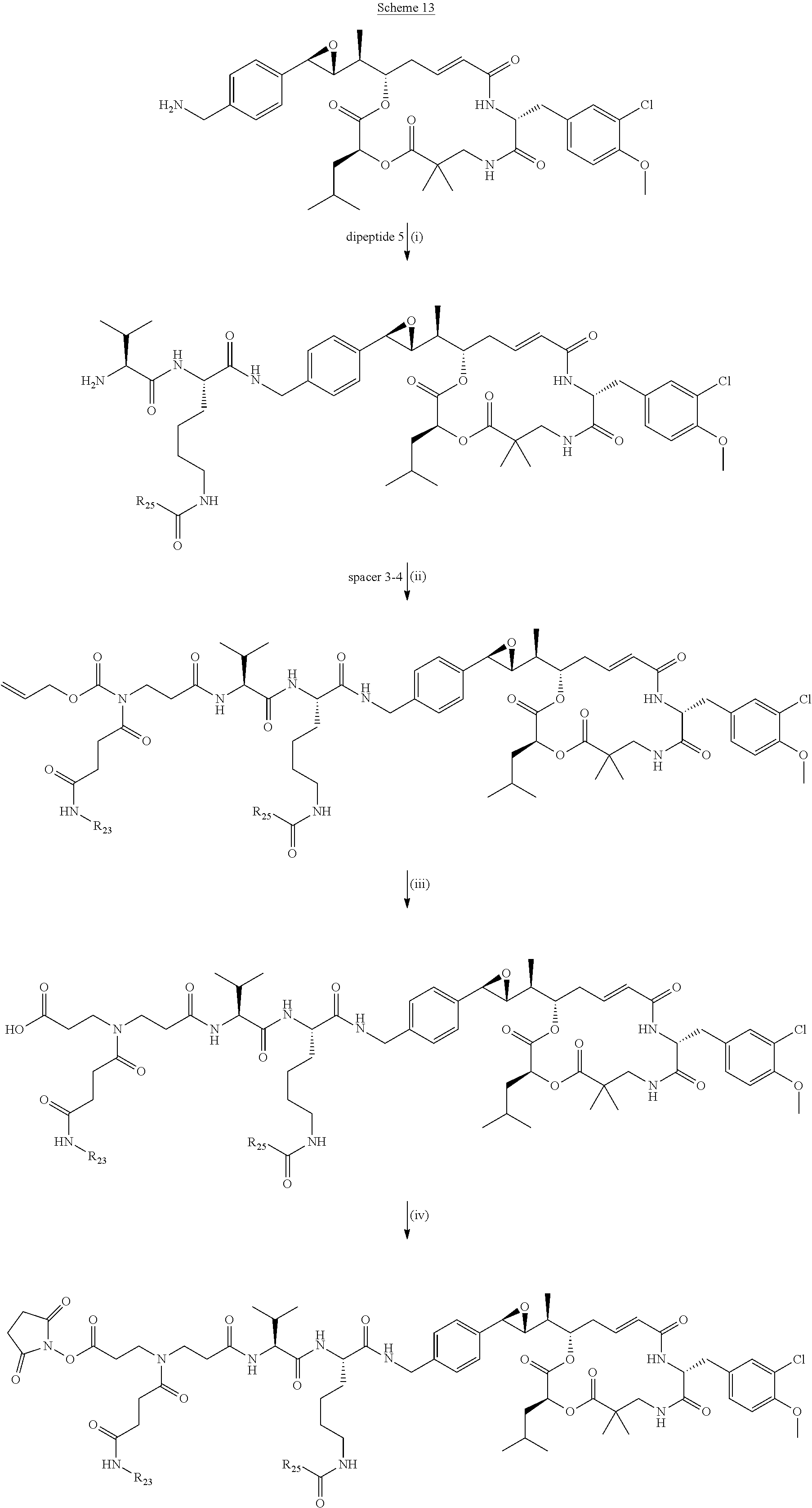

Step (i): peptidic coupling between the cryptophycin derivative and the dipeptide using coupling reagents such as, for example, EDC and HOBt and deprotection of the Fmoc amine in the presence of a base such as, for example, piperidine;
Step (ii): peptidic coupling with spacer 3 using coupling reagents such as, for example, EDC and HOBt, or coupling with spacer 4 in the presence of a base such as, for example, DIEA;
Step (iii): deprotection of the allyl ester in the presence of a catalyst such as, for example, tetrakis-(triphenylphosphine)palladium; one additional deprotection step of the side chain of substituted amino acid may be required prior to step (iv);
Step (iv): activation of the carboxylic acid as a NHS ester by treatment with DSC in the presence of a base such as, for example, DIEA.

Scheme 13 depicted the synthesis of payloads using the p-benzylic amine of C52 but may also apply to other cryptophycin compounds; it depicted the synthesis using dipeptide 5 but may also apply to dipeptide 1; it depicted the synthesis using spacers 3 or 4 but may also apply to spacers 1 or 2.

### With PABA Moiety

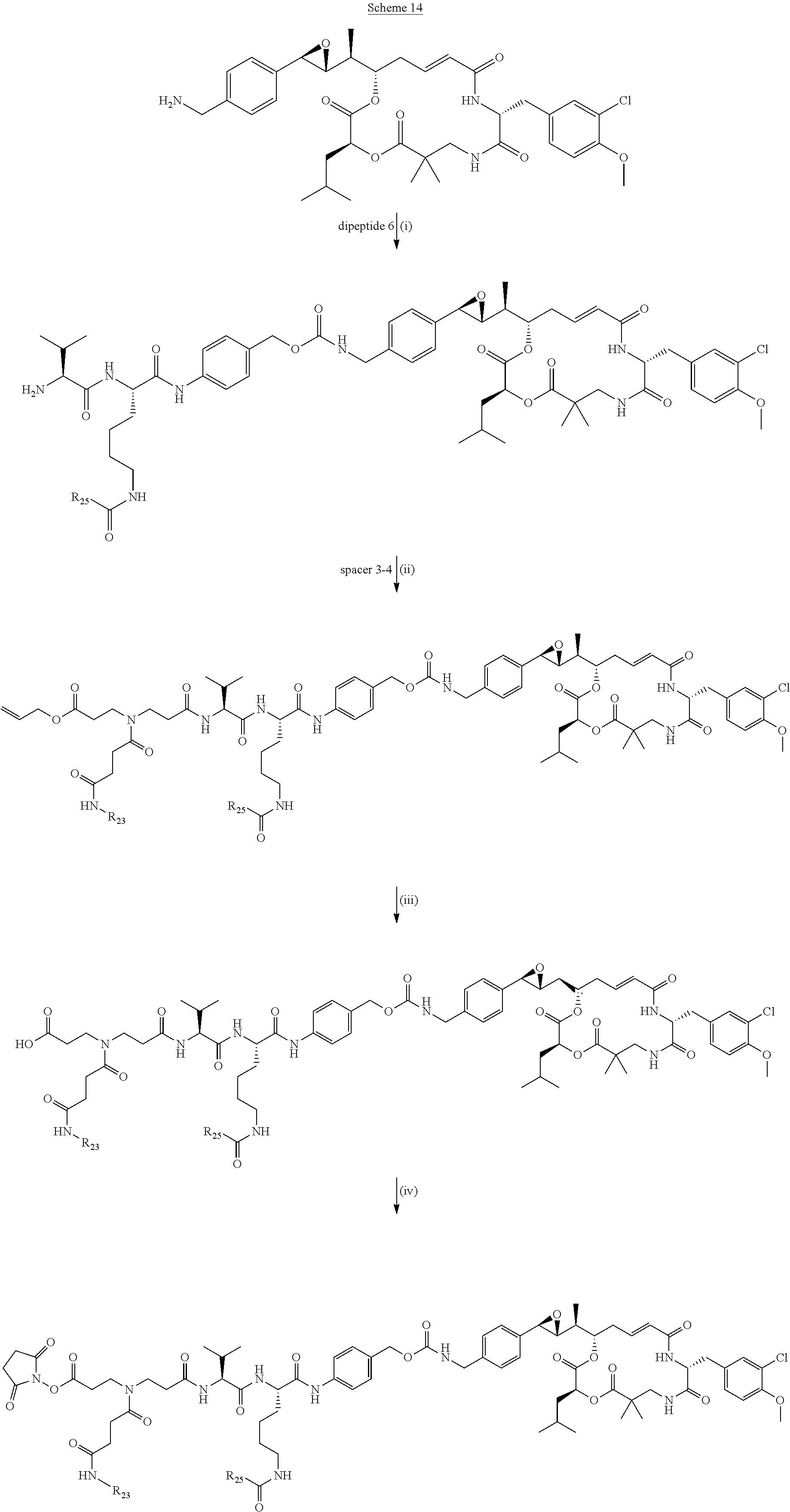

Step (i): formation of a carbamate between the activated alcohol and the cryptophycin amine in the presence of a base such as, for example, DIEA and deprotection of the Fmoc amine in the presence of a base such as, for example, piperidine;
Step (ii): peptidic coupling with spacer 3 using coupling reagents such as, for example, EDC and HOBt, or coupling with spacer 4 in the presence of a base such as, for example, DIEA;
Step (iii): deprotection of the allyl ester in the presence of a catalyst such as, for example, tetrakis-(triphenylphosphine)palladium; one additional deprotection step of the side chain of substituted amino acid may be required prior to step (iv);
Step (iv): activation of the carboxylic acid as a NHS ester by treatment with DSC in the presence of a base such as, for example, DIEA.

Scheme 14 depicted the synthesis of payloads using the p-benzylic amine of C52 but may also apply to other cryptophycin compounds; it depicted the synthesis using dipeptide 6 but may also apply to dipeptide 2; it depicted the synthesis using spacers 3 or 4 but may also apply to spacers 1 or 2.

Preparation of the new cryptophycin payloads of formula (IV) for Y═O—CH2 or S—CH2
Preparation of the New Cryptophycin Payloads Bearing a Spacer with Improved Hydrophilicity

### Without PABA Moiety

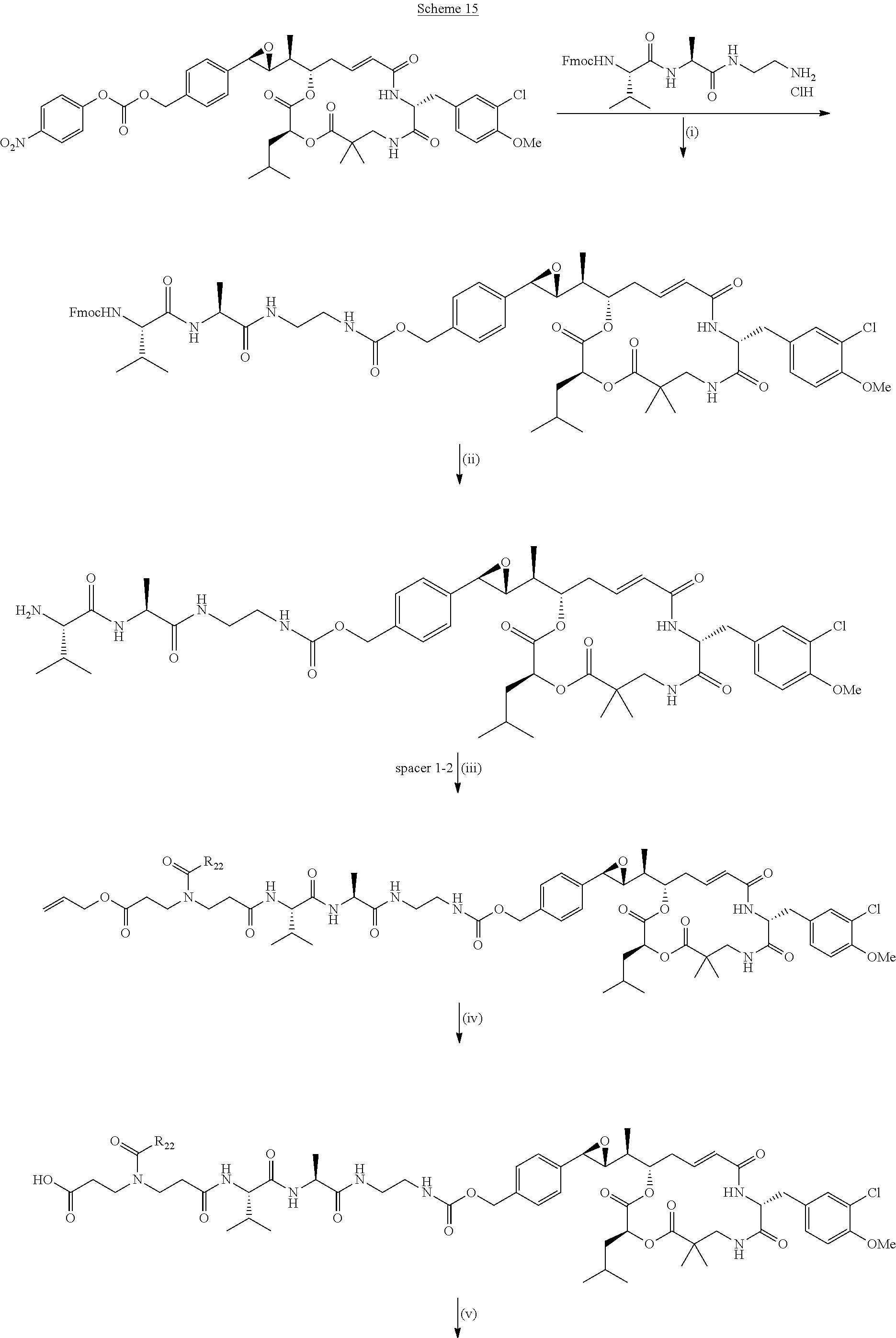

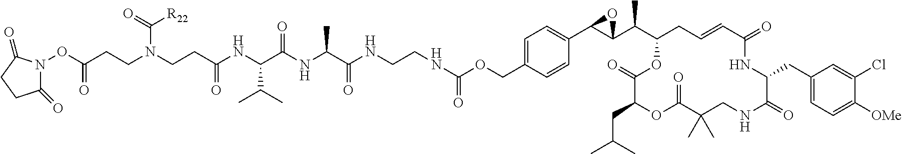

Step (i): formation of a carbamate between the cryptophycin activated alcohol and the amine in the presence of a base such as, for example, DIEA;
Step (ii): deprotection of the Fmoc amine in the presence of a base such as, for example, piperidine;
Step (iii): peptidic coupling with spacer 1 using coupling reagents such as, for example, EDC and HOBt. or coupling with spacer 2 in the presence of a base such as, for example, DIEA;
Step (iv): deprotection of the allyl ester in the presence of a catalyst such as, for example, tetrakis-(triphenylphosphine)palladium;
Step (v): activation of the carboxylic acid as a NHS ester by treatment with DSC in the presence of a base such as, for example, DIEA.

Scheme 15 depicted the synthesis of payloads using the activated p-benzylic alcohol of C52 but may also apply to other cryptophycin compounds; it depicted the synthesis using Val-Ala dipeptides but may also apply to other dipeptides; it depicted the synthesis using Boc-monoprotected ethylenediamine but may also apply to other Boc-monoprotected diamines which are commercially available for n ranging from 3 to 10; it depicted the synthesis using spacers 1 or 2 but may also apply to spacers 3 or 4.

Alternatively, new cryptophycin payloads of formula (IV) bearing a spacer with improved hydrophilicity may also be prepared as depicted in Scheme 16.

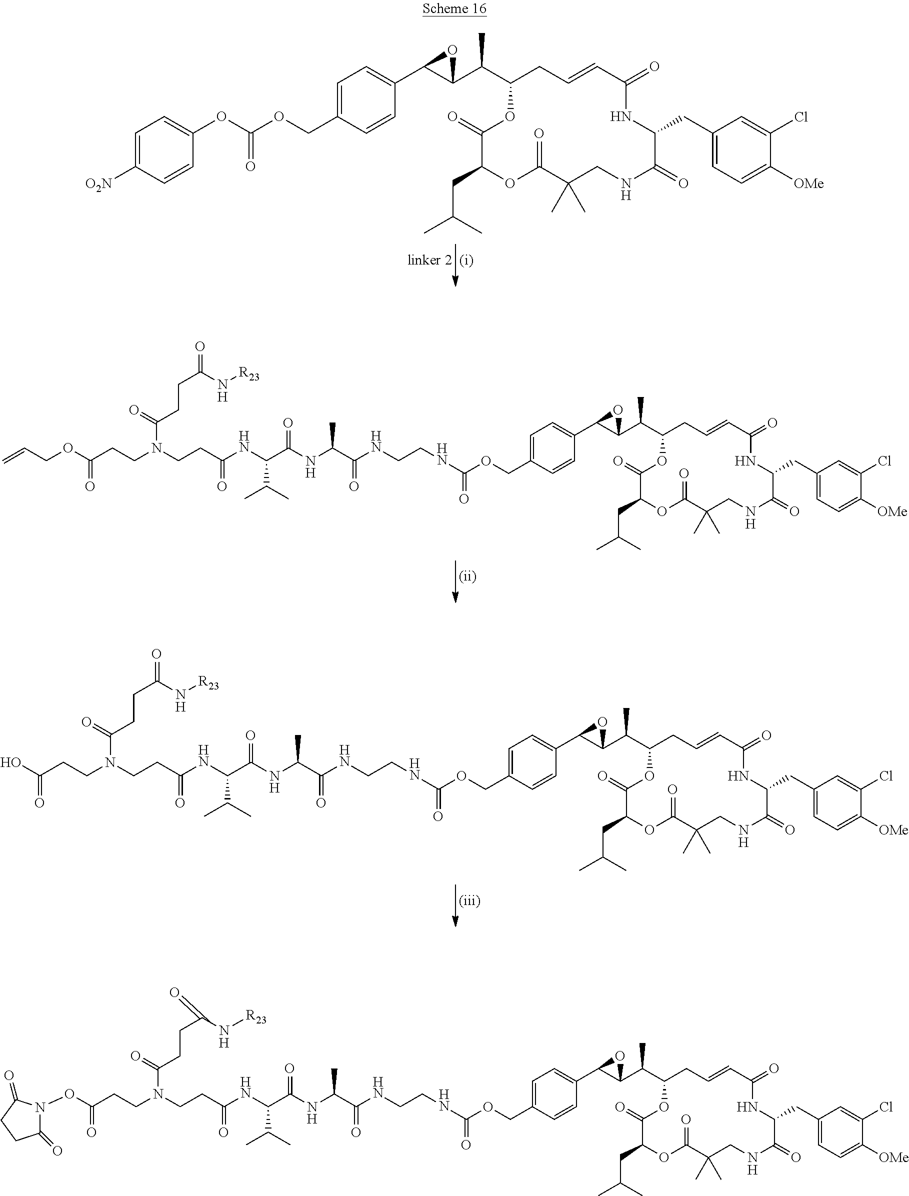

Step (i): formation of a carbamate between the cryptophycin activated alcohol and linker 2 in the presence of a base such as, for example, DIEA;
Step (ii): deprotection of the allyl ester in the presence of a catalyst such as, for example, tetrakis-(triphenylphosphine)palladium;
Step (iii): activation of the carboxylic acid as a NHS ester by treatment with DSC in the presence of a base such as, for example, DIEA.

Scheme 16 depicted the synthesis of payloads using the activated p-benzylic alcohol of C52 but may also apply to other cryptophycin compounds.

### With PABA Moeity

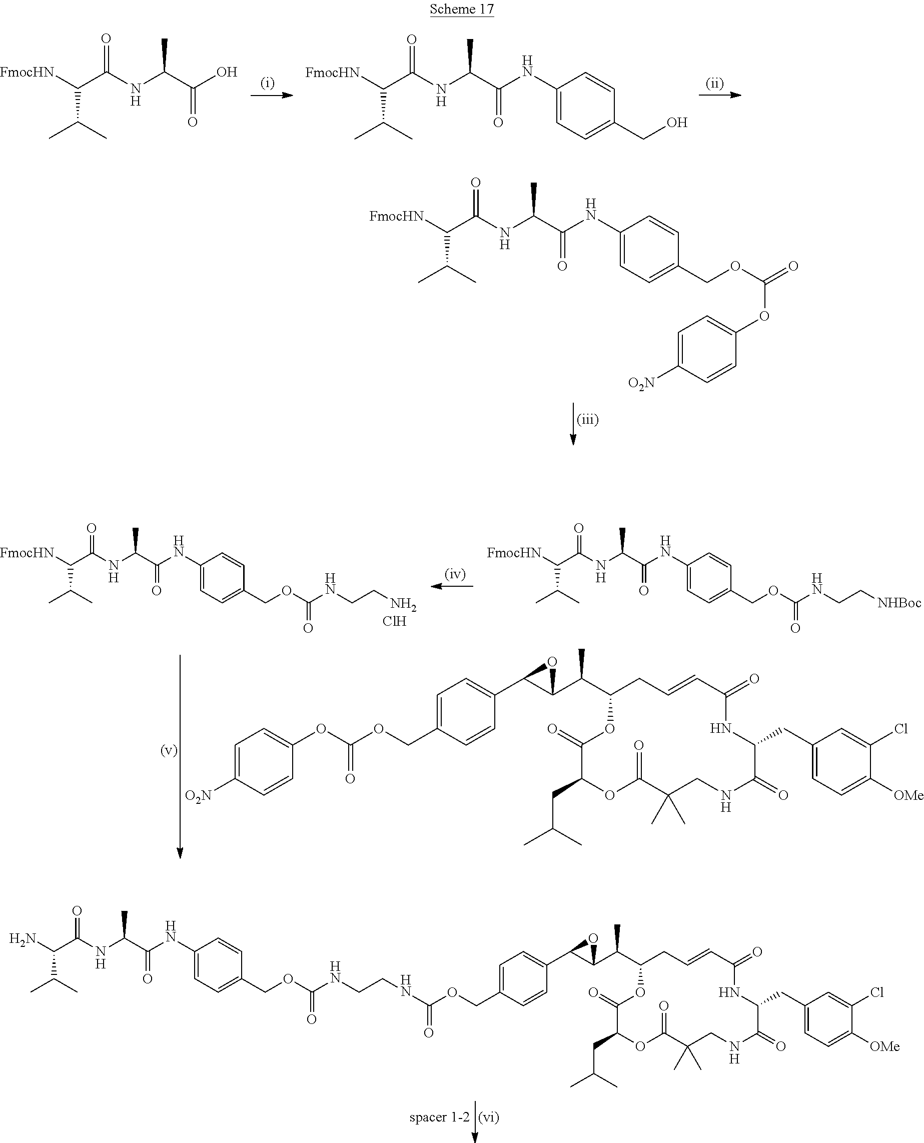

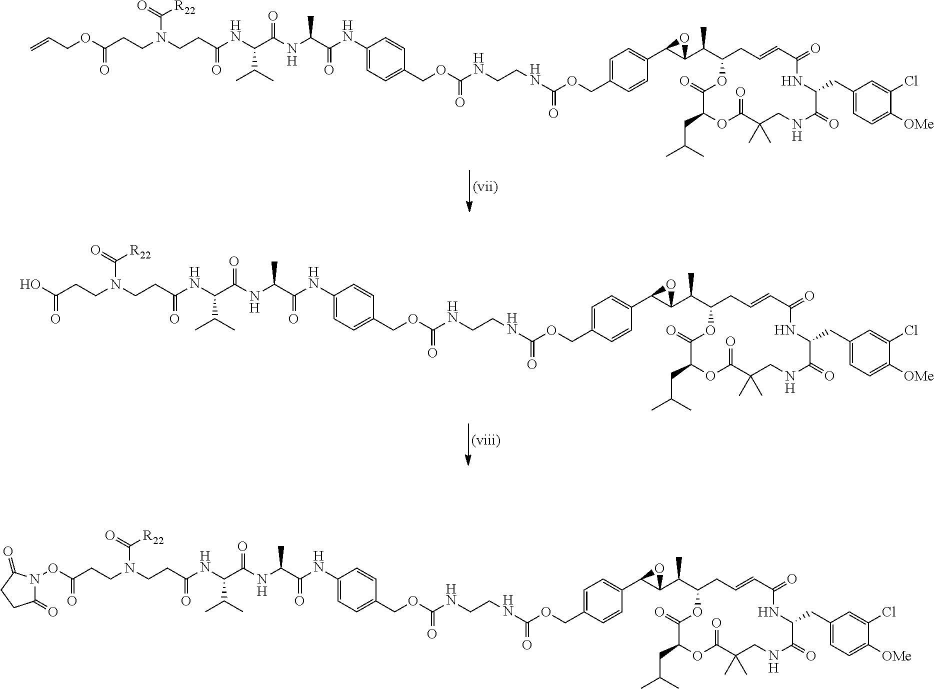

Step (i): peptidic coupling between Fmoc-Val-Ala-OH and 4-aminobenzyl alcohol in the presence of a coupling reagent such as, for example, EEDQ;
Step (ii): activation of the benzylic alcohol as a p-nitrophenyl carbonate by treatment with p-nitrophenyl-chloroformate in the presence of a base such as, for example, DIEA;
Step (iii): formation of a carbamate between the activated alcohol and Boc-monoprotected ethylene diamine in the presence of a base such as, for example, DIEA;
Step (iv): deprotection of the Boc amine using a solution of HCl (for example solution in dioxane) or of TFA;
Step (v): formation of a carbamate between the cryptophycin activated alcohol and the amine in the presence of a base such as, for example, DIEA and deprotection of the Fmoc amine in the presence of a base such as, for example, piperidine;
Step (vi): peptidic coupling with spacer 1 using coupling reagents such as, for example, EDC and HOBt, or coupling with spacer 2 in the presence of a base such as, for example, DIEA;
Step (vii): deprotection of the allyl ester in the presence of a catalyst such as, for example, tetrakis-(triphenylphosphine)palladium;
Step (viii): activation of the carboxylic acid as a NHS ester by treatment with DSC in the presence of a base such as, for example, DIEA.

Scheme 17 depicted the synthesis of payloads using Val-Ala dipeptides but may also apply to other dipeptides; it depicted the synthesis using p-aminobenzyl alcohol (CAS number [623-04-1]) but may also apply to other aminobenzyl alcohol compounds which are commercially available such as, for example, 4-(1-hydroxyethyl)-aniline (racemic (CAS number [14572-89-5]) or enantiopure (R) (CAS number [210754-25-9]) or (S) (CAS number [500229-84-5])), 4-amino-α,α-dimethyl-benzene-methanol (CAS number [23243-04-1]), 4-amino-α-methoxy-α-methyl-benzenemethanol (CAS number [1379318-81-6]), 4-amino-α-methyl-α-trifluoromethyl-benzenemethanol (CAS number [851652-56-7]), 2-amino-benzenemethanol (CAS number [5344-90-1]), 2-amino-α-methyl-benzenemethanol (racemic (CAS number [10517-50-7]) or enantiopure (R) (CAS number [3205-21-8]) or (S) (CAS number [3205-21-8])), 6-amino-3-pyridinemethanol (CAS number [113293-71-3]), 6-amino-α-methyl-3-pyridinemethanol (CAS number [1335054-83-5]), 6-amino-α-ethyl-3-pyridinemethanol (CAS number [1355225-85-2]), 6-amino-α,α-dimethyl-3-pyridinemethanol (CAS number [843646-03-8]), 5-amino-3-pyridinemethanol (CAS number [873651-92-4]), 2-amino-3-pyridinemethanol (CAS number [23612-57-9]), 2-amino-α-methyl-3-pyridinemethanol (racemic (CAS number [869567-91-9]) or enantiopure (R) (CAS number [936718-01-3]) or (S) (CAS number [936718-00-2])), 2-amino-α-ethyl-3-pyridinemethanol (CAS number [914223-90-8]), 2-amino-α,α-dimethyl-3-pyridinemethanol (CAS number [213666-96-7]), 3-amino-4-pyridinemethanol (CAS number [152398-05-5]), 3-amino-α-methyl-4-pyridinemethanol (CAS number [1242470-88-7]), 3-amino-α,α-methyl-4-pyridinemethanol (CAS number [13357-81-8]), 4-amino-3-pyridinemethanol (CAS number [138116-34-4]), 4-amino-α-methyl-3-pyridinemethanol (CAS number [741223-49-4]), 4-amino-α,α-methyl-3-pyridinemethanol (CAS number [1339013-26-1]), 3-amino-2-pyridinemethanol (CAS number [52378-63-9]), 3-amino-α-methyl-2-pyridinemethanol (CAS number [954240-54-1]), 3-amino-α,α-methyl-2-pyridinemethanol (CAS number [899438-57-4]); it depicted the synthesis using Boc-monoprotected ethylenediamine but may also apply to other Boc-monoprotected diamines which are commercially available for n ranging from 3 to 10; it depicted the synthesis using the activated p-benzylic alcohol of C52 but may also apply to other cryptophycin compounds; it depicted the synthesis using spacers 1 or 2 but may also apply to spacers 3 or 4.

Preparation of the New Cryptophycin Payloads of Formula (IV) Bearing a Dipeptide with Improved Hydrophilicity

### Without PABA Moiety

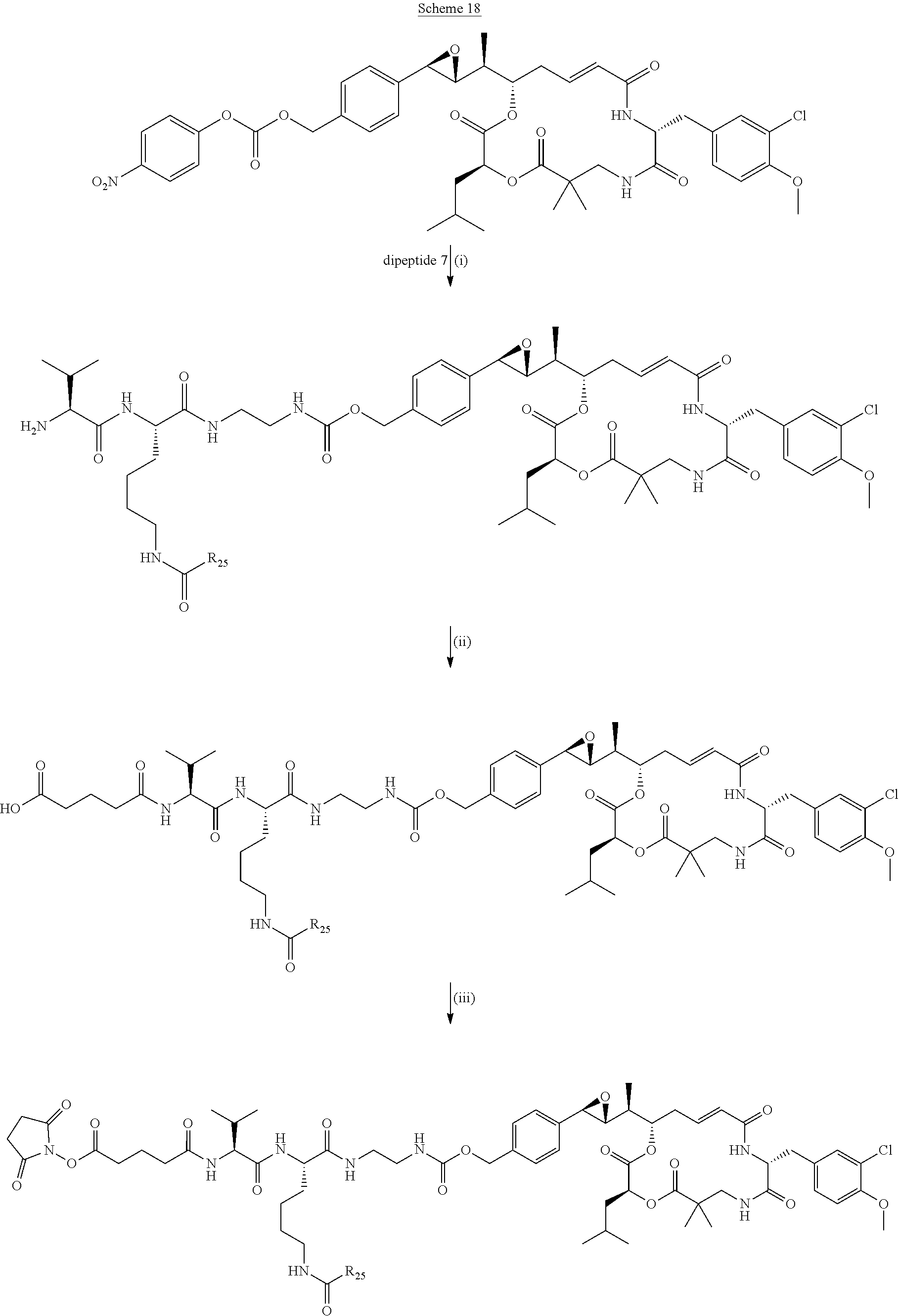

Step (i): formation of a carbamate between the cryptophycin activated alcohol and the dipeptide in the presence of a base such as, for example, DIEA and deprotection of the Fmoc amine in the presence of a base such as, for example, piperidine;
Step (ii): coupling with glutaric anhydride; one additional deprotection step of the side chain of substituted amino acid may be required prior to step (iii)
Step (iii): activation of the carboxylic acid as a NHS ester by treatment with DSC in the presence of a base such as, for example, DIEA.

Scheme 18 depicted the synthesis of payloads using the activated p-benzylic alcohol of C52 but may also apply to other cryptophycin compounds; it depicted the synthesis using dipeptide 7 but may also apply to dipeptide 3; it depicted the synthesis using glutaric anhydride but may also apply to succinic anhydride or alkyl diacids which are commercially available for n ranging from 3 to 10.

### With PABA Moiety

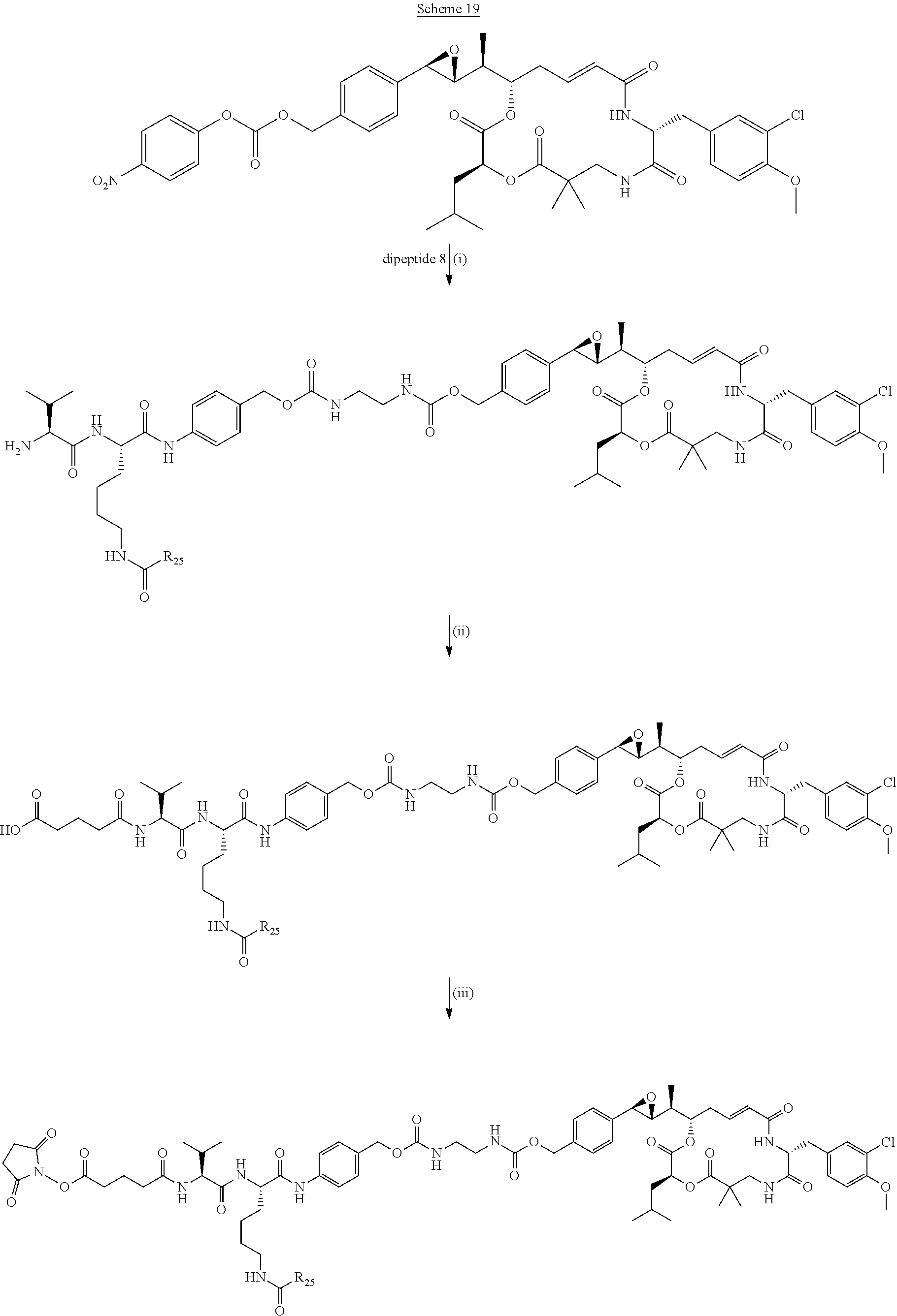

Step (i): formation of a carbamate between the cryptophycin activated alcohol and the dipeptide in the presence of a base such as, for example, DIEA and deprotection of the Fmoc amine in the presence of a base such as, for example, piperidine;
Step (ii): coupling with glutaric anhydride; one additional deprotection step of the side chain of substituted amino acid may be required prior to step (iii)
Step (iii): activation of the carboxylic acid as a NHS ester by treatment with DSC in the presence of a base such as, for example, DIEA.

Scheme 19 depicted the synthesis of payloads using the activated p-benzylic alcohol of C52 but may also apply to other cryptophycin compounds; it depicted the synthesis using dipeptide 8 but may also apply to dipeptide 4; it depicted the synthesis using glutaric anhydride but may also apply to succinic anhydride or alkyl diacids which are commercially available for n ranging from 3 to 10.

Preparation of the New Cryptophycin Payloads of Formula (IV) Bearing Both a Spacer and a Dipeptide with Improved Hydrophilicity

### Without PABA Moeity

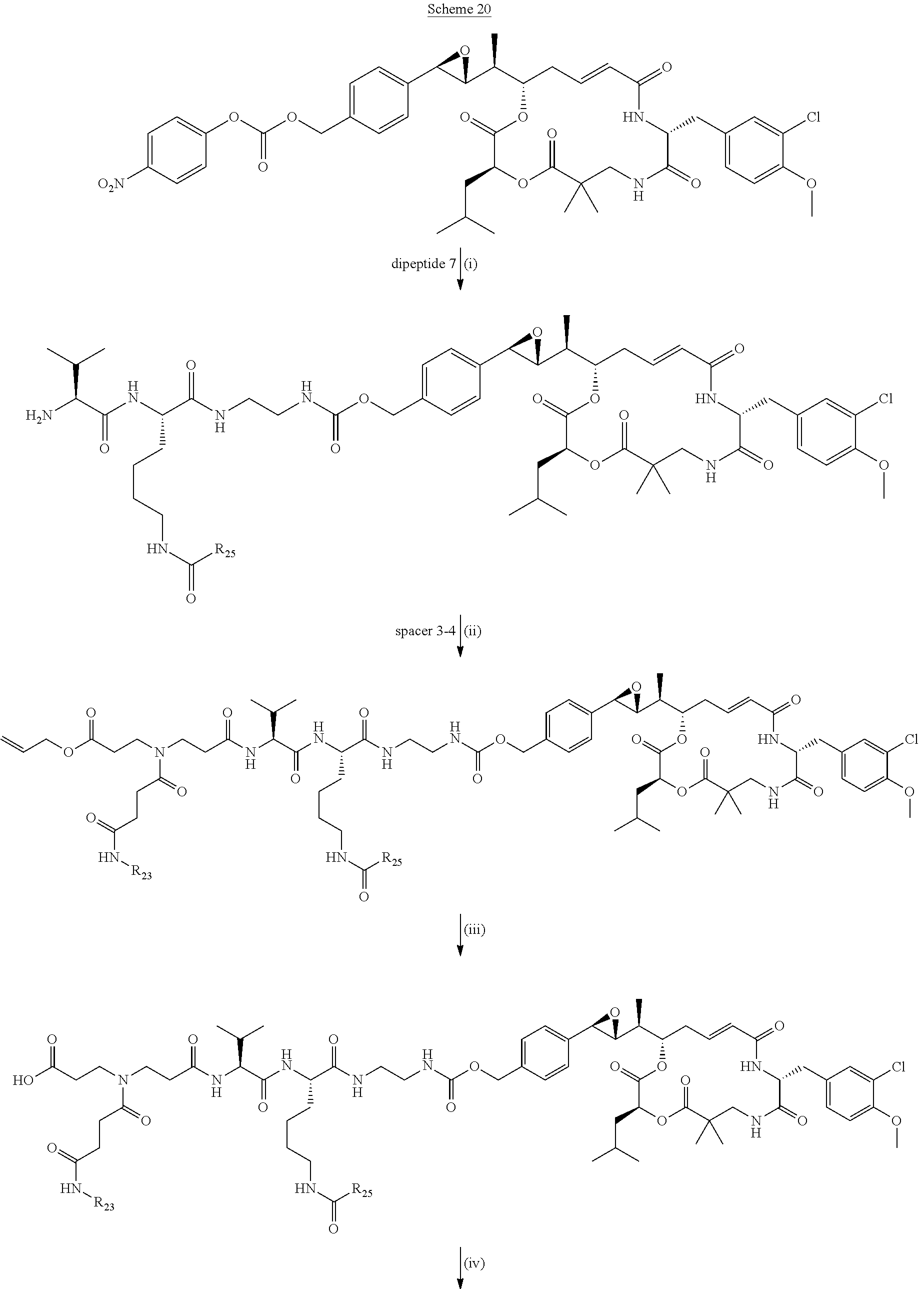

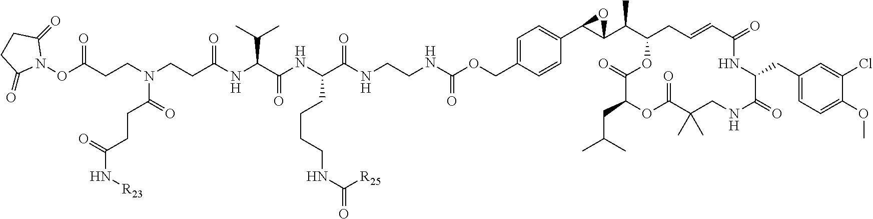

Step (i): formation of a carbamate between the cryptophycin activated alcohol and the dipeptide in the presence of a base such as, for example, DIEA and deprotection of the Fmoc amine in the presence of a base such as, for example, piperidine;
Step (ii): peptidic coupling with spacer 3 using coupling reagents such as, for example, EDC and HOBt, or coupling with spacer 4 in the presence of a base such as, for example, DIEA;
Step (iii): deprotection of the allyl ester in the presence of a catalyst such as, for example, tetrakis-(triphenylphosphine)palladium; one additional deprotection step of the side chain of substituted amino acid may be required prior to step (iv)
Step (iv): activation of the carboxylic acid as a NHS ester by treatment with DSC in the presence of a base such as, for example, DIEA.

Scheme 20 depicted the synthesis of payloads using the p-benzylic amine of C52 but may also apply to other cryptophycin compounds; it depicted the synthesis using dipeptide 7 but may also apply to dipeptide 3; it depicted the synthesis using spacers 3 or 4 but may also apply to spacers 1 or 2.

### With PABA Moiety

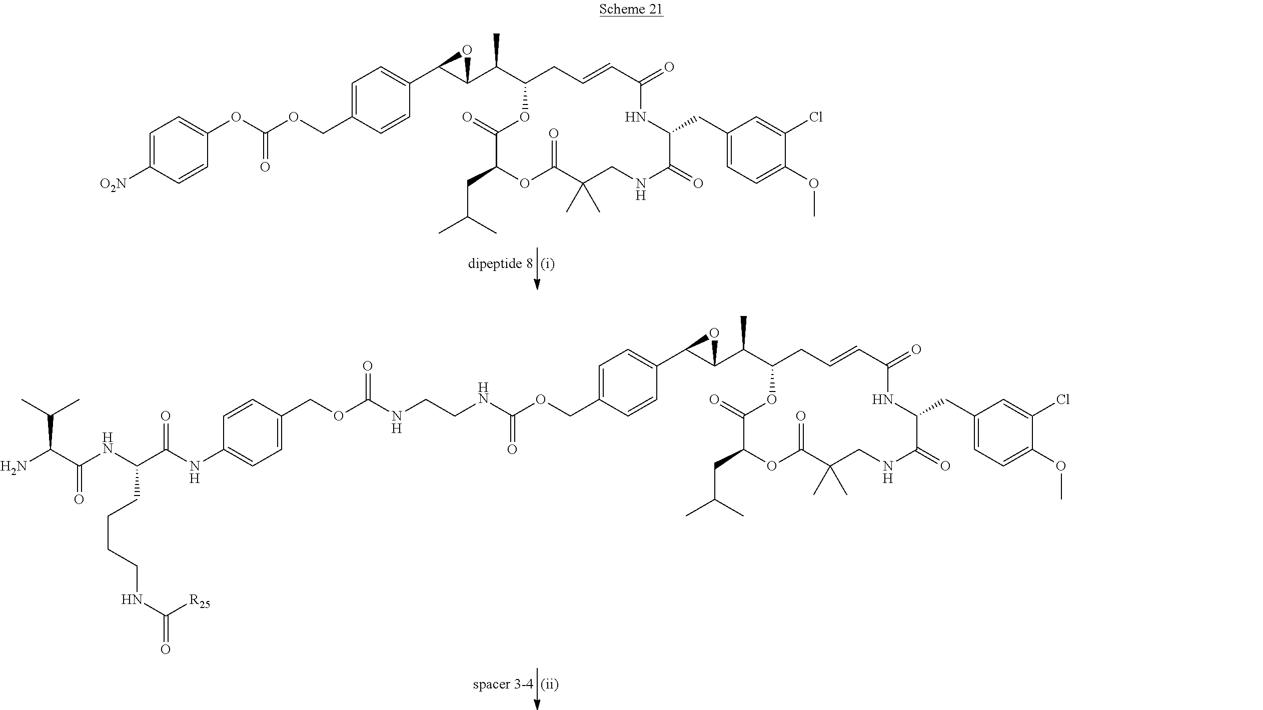

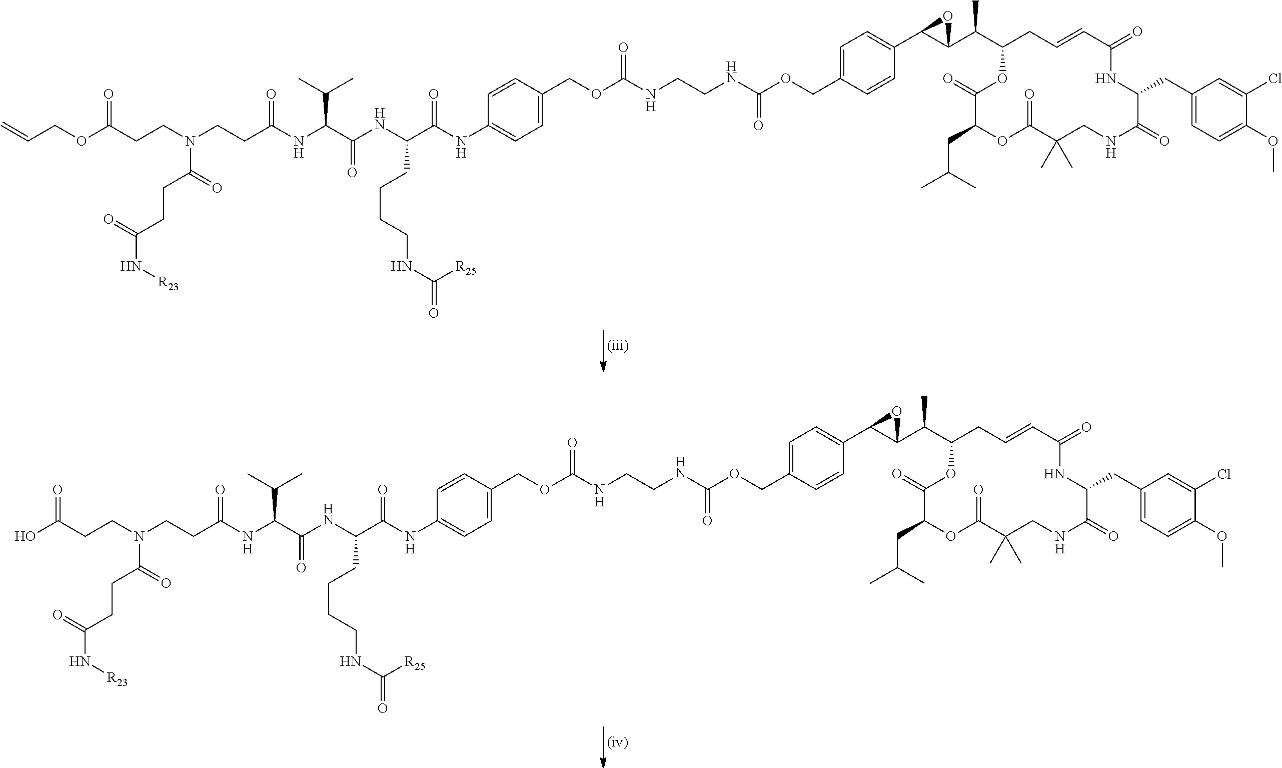

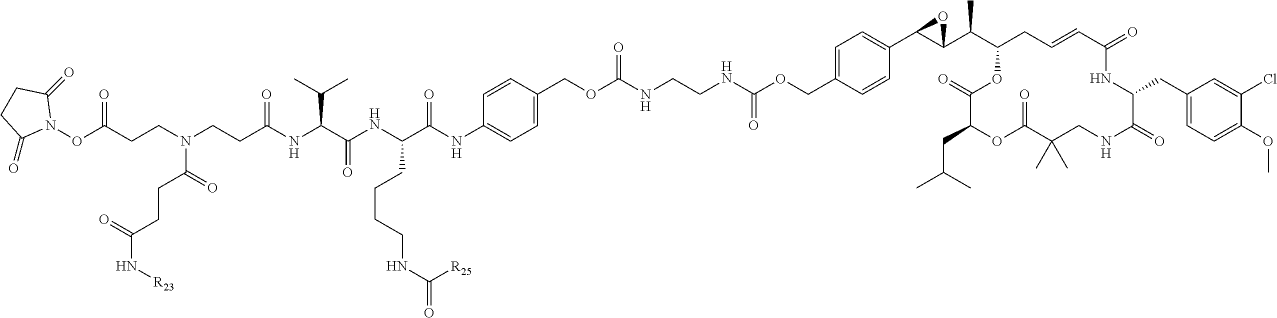

Step (i): formation of a carbamate between the cryptophycin activated alcohol and the dipeptide in the presence of a base such as, for example, DIEA and deprotection of the Fmoc amine in the presence of a base such as, for example, piperidine;
Step (ii): peptidic coupling with spacer 3 using coupling reagents such as, for example, EDC and HOBt, or coupling with spacer 4 in the presence of a base such as, for example, DIEA;
Step (iii): deprotection of the allyl ester in the presence of a catalyst such as, for example, tetrakis-(triphenylphosphine)palladium; one additional deprotection step of the side chain of substituted amino acid may be required prior to step (iv);
Step (iv): activation of the carboxylic acid as a NHS ester by treatment with DSC in the presence of a base such as, for example, DIEA.

Scheme 21 depicted the synthesis of payloads using the p-benzylic amine of C52 but may also apply to other cryptophycin compounds; it depicted the synthesis using dipeptide 8 but may also apply to dipeptide 4; it depicted the synthesis using spacers 3 or 4 but may also apply to spacers 1 or 2.

### Preparation of the Conjugates of Formula (V)

The conjugates of formula (V) of the present invention can obtained via the process comprising at least the steps of:
(i) placing in contact and leaving to react:
   an optionally buffered aqueous solution of an antibody, optionally modified by means of a modifying agent, and
   a solution of a cryptophycin payload of formula (IV) as defined in the present invention,
the chemical group RCG1 of the cryptophycin payload of formula (IV) being reactive towards the chemical groups RCG2 present on the polypeptide such as the antibody especially towards the amino groups present on antibodies, the said chemical groups RCG2 having been introduced, where appropriate, by the modifying agent,
so as to attach the cryptophycin payload of formula (IV) to the antibody by formation of a covalent bond;
(ii) and then optionally to separate the conjugate of formula (V) formed in step (i) from the cryptophycin payload of formula (IV) and/or from the unreacted antibody and/or from any aggregates that may have formed.

According to one variant for example, in step (ii) the conjugate of formula (V) from step
(i) is separated only from the unreacted cryptophycin payload of formula (IV) and from any aggregates formed, and any unreacted antibody is left in the solution.

The function of the placing in contact is to react the chemical groups RCG1 and RCG2 in order to ensure attachment of the cryptophycin payload of formula (IV) to the polypeptide such as the antibody by formation of a covalent bond; such as,
when RCG1 represents RaZa-C(═O), the reaction preferably takes place on the amino functions of the antibody, such as the 8-amino groups borne by the side chains of the lysine (Lys) residues of the antibody and the α-amino groups of N-terminal amino acids of antibody heavy and light chains. A conjugate of the following formula is obtained in this case: mAb-[NH—C(═O)-L*—Y-Crypto]ₐ with L* representing a fragment of a linker L comprising as RCG1 a RaZa-C(═O) group and d representing the drug-to-antibody ratio or DAR;
when RCG1 represents a chlorine atom or a maleimido or haloacetamido group, the antibody may comprise thiol chemical groups;
when RCG1 represents an azido group, the antibody may comprise a C≡CH moiety or an activated triple bond such as a cyclooctyne group;
when RCG1 represents NH₂ group, the reaction may take place on amide function of the antibody using an enzymatic catalysis, such as the amide groups borne by the side chains of glutamine (Gln) residues of an antibody. A conjugate of the following formula is obtained in this case: mAb-[C(═O)—NH—L*-Crypto]ₐ with L* representing a fragment of a linker L comprising as RCG1 a NH₂ group and such that L representing a L*NH₂ group and d representing the drug-to-antibody ratio or DAR;
when RCG1 represents a C≡CH group or an activated C≡C group such as a cyclooctyne moiety, the antibody may comprises azido groups.

The term “aggregates” means associations that may form between two or more antibodies, the antibodies possibly having been modified by conjugation. Aggregates are liable to form under the influence of a wide variety of parameters such as a high concentration of antibody in the solution, the pH of the solution, high shear forces, the number of grafted drugs and their hydrophobic nature, the temperature (see the references cited in the introduction of J. Membrane Sci. 2008, 318, 311-316), the influence of some of them, however, having not been clearly elucidated. In the case of proteins or antibodies, reference may be made to AAPS Journal, “Protein Aggregation and Bioprocessing” 2006, 8(3), E572-E579. The aggregate content may be determined via known techniques such as SEC (see in this respect Analytical Biochemistry 1993, 212 (2), 469-480).

The aqueous solution of the antibody may be buffered with buffers for example, potassium phosphate or HEPES or a mixture of buffers such as buffer A described later. The buffer depends on the nature of the antibody. The cryptophycin payload of formula (IV) is dissolved in a polar organic solvent such as DMSO or DMA.

The reaction takes place at a temperature generally ranging from 20° C. to 40° C. The reaction time may be ranging from 1 to 24 hours. The reaction between the antibody and the cryptophycin payload of formula (IV) may be monitored by SEC with a refractometric and/or ultraviolet detector and/or HRMS in order to determine its degree of progress. If the degree of substitution is insufficient, the reaction can be left for longer and/or cryptophycin compound can be added. Reference may be made to the example section for further details regarding particular conditions. Particular embodiments are described in Examples 3, 6, 16, 19, 23, 26, 29, 32, 35 and 41.

A person skilled in the art has at his disposal various chromatographic techniques for the separation of step (ii): the conjugate may be purified, for example, by steric exclusion chromatography (SEC), by adsorption chromatography (for instance ion exchange, IEC), by hydrophobic interaction chromatography (HIC), by affinity chromatography, by chromatography on mixed supports such as ceramic hydroxyapatite, or by HPLC. Purification by dialysis or diafiltration may also be used.

After step (i) or (ii), the solution of the conjugate may undergo an ultrafiltration and/or diafiltration step (iii). After these steps, the conjugate in aqueous solution is thus obtained.

### Antibody

The antibody can be a monoclonal antibody selected from the group consisting of a murine, chimeric, a humanized and a human antibody.

In one embodiment, the antibody is a monospecific antibody, i.e. an antibody specifically binding to one single target. Alternatively, it might be a multispecific antibody.

In one embodiment, the antibody is a IgG antibody, for instance an IgG₁, an IgG₂, an IgG₃ or an IgG₄ antibody.

The antibody according to the invention specifically binds to a target, thereby directing the biologically active compound as a cytotoxic compound towards said target. As used herein, “specifically binds” or “binds specifically to” or “binds to” or the like, means that an antibody or antigen-binding fragment thereof forms a complex with an antigen that is relatively stable under physiological conditions. Specific binding can be characterized by an equilibrium dissociation constant (K_{D}) of at least about 1×10⁸ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been characterized, for example, by their specific binding to target and/or target antigen using surface plasmon resonance, e.g., BIACORE™.

The target typically corresponds to a protein expressed at the cell surface, e.g. a protein expressed at the surface of tumour cells.

In one embodiment, the target is the EphA2 receptor. The EphA2 receptor is an Ephrin receptor, and is also referred to as “Eph receptor A2” or “Epithelial Cell Receptor Protein-Tyrosine kinase”. The antibody specifically binding to the EphA2 receptor might for instance correspond to one of the antibodies described in WO2008/010101 or WO2011/039724.

The antibody may optionally be modified with a modifying agent so as to promote the attachment of the cryptophycin payload as previously described. The antibody may especially be monoclonal, polyclonal or multispecific. It may also be an antibody fragment. It may also be a murine, human, humanized or chimeric antibody. The antibody used in the examples of the present invention is hu2H11_R3574, an antagonist antibody against EphA2 receptor. The sequence of hu2H11_R3574 is represented by SEQ ID NO: 1 (light chain of antibody hu2H11_R35R74) and by SEQ ID NO:2 (heavy chain of antibody hu2H11_R35R74) which correspond to respectively SEQ ID NO: 16 and SEQ ID NO:18 represented in WO2011039724 A1.

### Conjugate

A conjugate generally comprises from about 1 to 10 cryptophycin compounds covalently attached to the antibody (this is the degree of grafting or “drug-to-antibody ratio” or “DAR”). This number varies as a function of the nature of the antibody and of the cryptophycin compound, and also of the operating conditions used in the conjugation process (for example the number of equivalents of cryptophycin compound relative to the antibody, the reaction time, the nature of the solvent and of any cosolvent). Placing of the antibody and the cryptophycin compound in contact leads to a mixture comprising several conjugates that are individually distinguished from each other by different DARs; optionally the unreacted antibody; optionally aggregates. The DAR that is determined on the final solution thus corresponds to an average DAR. The DAR may be calculated from the deconvolution of the SEC-HRMS spectrum of the conjugate. The DAR (HRMS) is for example greater than 0.5, for instance ranging from 1 to 10, such as ranging from 2 to 7.

The conjugate may be used as an anticancer agent. Owing to the presence of the antibody, the conjugate is made highly selective towards tumor cells rather than healthy cells. This makes it possible to direct the cryptophycin compound in an environment similar thereto or directly therein. It is possible to treat solid or liquid cancers. The conjugate may be used alone or in combination with at least one other anticancer agent.

The conjugate is formulated in the form of a buffered aqueous solution at a concentration generally ranging from 1 to 10 mg/mL. This solution may be injected in perfusion form per se or may be rediluted to form a perfusion solution.

### EXAMPLES

The examples which follow describe the preparation of certain compounds in accordance with the invention. These examples are not limitative, and merely illustrate the present invention.

### Analytical Methods Used

### High Pressure Liquid Chromatography—Mass Spectrometry (LCMS)

### Method a

Spectra have been obtained on a Waters UPLC-SQD system in positive and/or negative electrospray mode (ES+/−). Chromatographic conditions were the following:
Column: ACQUITY BEH C18—1.7 μm—2.1×50 mm; solvents: A: H₂O (0.1% formic acid), B: CH₃CN (0.1% formic acid); column temperature: 50° C.; flow rate: 0.8 mL/min; gradient (2.5 min): from 5 to 100% of B in 1.8 min; 2.4 min: 100% of B; 2.45 min: from 100 to 5% of B in 0.05 min.

### Method B

Spectra have been obtained on a Waters UPLC-SQD system in positive and/or negative electrospray mode (ES+/−). Chromatographic conditions were the following:
Column: ACQUITY BEH C18—1.7 μm—2.1×50 mm; solvents: A: H₂O (0.1% formic acid), B: CH₃CN (0.1% formic acid); column temperature: 50° C.; flow rate: 0.6 mL/min; gradient (2 min): from 5 to 50% of B in 1 min; from 50 to 100% of B in 0.3 min; 100% of B during 0.15 min; from 100 to 5% de B in 0.3 min and 5% of B during 0.25 min.

### Method C

Spectra have been obtained on a Waters UPLC-SQD system in positive and/or negative electrospray mode (ES+/−). Chromatographic conditions were the following:
Column: ACQUITY BEH C18—1.7 μm—2.1×50 mm; solvents: A: H₂O (0.1% formic acid), B: CH₃CN (0.1% formic acid); column temperature: 50° C.; flow rate: 0.8 mL/min; gradient (5 min): from 5 to 100% of B in 4.2 min; 4.6 min: 100% of B; 4.8 min: 5% of B.

### Method D

Spectra have been obtained on a Waters XeVo—Qtof system in positive electrospray mode (ES+). Chromatographic conditions were the following:
Column: ACQUITY BEH C18—1.7 μm—2.1×100 mm; solvents: A: H₂O (0.1% formic acid), B: CH₃CN (0.1% formic acid); column temperature: 45° C.; flow rate: 0.6 mL/min; gradient (5.3 min): 5% of B from 0 to 0.3 min, 4 min: 100% of B; 4.6 min: 100% of B; 5.3 min: 5% of B.

### Method E

Spectra have been obtained on a Waters UPLC-SQD system in positive and/or negative electrospray mode (ES+/−). Chromatographic conditions were the following:
Column: ACQUITY BEH C18—1.7 μm—2.1×50 mm; solvents: A: H₂O (0.1% formic acid), B: CH₃CN (0.1% formic acid); column temperature: 45° C.; flow rate: 0.8 mL/min; gradient (10 min): from 5 to 100% of B in 8.6 min; 9.6 min: 100% of B; 9.8 min: 5% of B.

### ¹H Nuclear Magnetic Resonance (NMR)

The ¹H NMR spectra were acquired on a Bruker Avance spectrometer, either of model DRX-300, DRX-400 or DRX-500. The chemical shifts (6) are given in ppm.

### Size Exclusion Chromatography—High Resolution Mass Spectrometry (SEC-HRMS)

The chromatographic analysis was performed on an Agilent HP1100 machine and a Waters BEH SEC 200 1.7 μm (2.1×150 mm) column at 30° C. with a flow rate of 0.5 mL/min and an isocratic elution of (A) 25 mM ammonium formate+1% formic acid/(B) CH₃CN+0.1% formic acid 70/30 for 15 minutes. The mass spectrometry was performed on a Waters QTOF-II machine with electrospray ionization in positive mode (ES+). The mass spectra were deconvoluted with the Waters MaxEnt1 software.

### Analytical Size Exclusion Chromatography (SEC)

The analysis was performed on a Waters Alliance HPLC system or a Hitachi Lachrom HPLC system equipped with a photodiode array detector and a Tosoh Bioscience TSKgel G3000 SWXL 5 μm column (7.8×300 mm) with a flow rate of 0.5 mL/min and an isocratic elution of 30 minutes with a pH 7 buffer containing 0.2 M of KCl, 0.052 M of KH₂PO₄, 0.107 M of K₂HPO₄ and 20% by volume of isopropanol.

### Buffers

Buffer A (pH 6.5): NaCl (50 mM), potassium phosphate buffer (50 mM), EDTA (2 mM)
Buffer B (pH 6.5): NaCl (140 mM), potassium and sodium phosphate (9.6 mM)
DPBS (pH 7.2): KCl (2.7 mM), NaCl (137 mM), KH₂PO₄ (1.47 mM), Na₂HPO₄ (8.10 mM)
PBS (pH 7.4): KH₂PO₄ (1.06 mM), NaCl (155.17 mM), Na₂HPO₄-7H₂O (2.97 mM)

### General Method Used for the Preparation of Antibody-Drug Conjugate (ADC)

A solution of antibody in an aqueous buffer composed of a 96:4 mixture of buffer A and 1 N HEPES was treated with an excess (5 to 10 equivalents) of a solution at approximatively 10 mM of cryptophycin payload in DMA such that the final antibody concentration is 3 mg/mL and the percentage of DMA in the aqueous buffer is 20%. After stirring for 1 to 4 hours, the mixture was analysed by SEC-HRMS to determine the DAR on the population of monomeric antibodies. If the DAR was found insufficient (<3.5-4), the mixture was treated with a further excess (1 to 5 equivalents) of cryptophycin solution in DMA for 2 to 4 additional hours at RT under stirring. The mixture was purified by gel filtration using a Superdex 200 pg matrix (HiLoad 16/60 or 26/60 desalting column, GEHealthcare) pre-equilibrated in aqueous buffer pH 6.5 (buffer B or DPBS) containing 10 to 20% of NMP or a Sephadex™ G25 matrix (Hiprep 26/10 desalting column, GEHealthcare) pre-equilibrated in aqueous buffer pH 6.5 (buffer B or DPBS) containing 5 to 10% of NMP. The fractions containing the monomeric conjugated antibody were pooled and concentrated on Amicon Ultra-15 (10 k or 50 k Ultracel membrane, Millipore) to a concentration of between 2 and 5 mg/mL. A buffer exchange or a dilution in the appropriate buffer was then performed to formulate the conjugate in the final buffer. In the case of a buffer exchange, it was realized by gel filtration using a Sephadex™ G25 matrix (NAP-5, NAP-10, NAP-25/PD-10 or Hiprep 26/10 desalting columns, GEHealthcare) pre-equilibrated with the final aqueous buffer whose composition and pH are suited to each conjugate. The conjugate was finally filtered through a Steriflip® filter unit (0.22 μm Durapore® PVDF membrane, Millipore). The final conjugate was assayed by UV spectrometry or SEC-HPLC so as to measure the conjugate concentration, by SEC-HPLC so as to determine the monomeric purity and by SEC-HRMS so as to determine the DAR from the deconvolution of the mass spectrum of the conjugate.

### Synthesis of Examples 1 to 3: PEG4-Val-Ala-C52 benzylic amine, NHS ester of PEG4-Val-Ala-C52 benzylic amine and Corresponding ADC

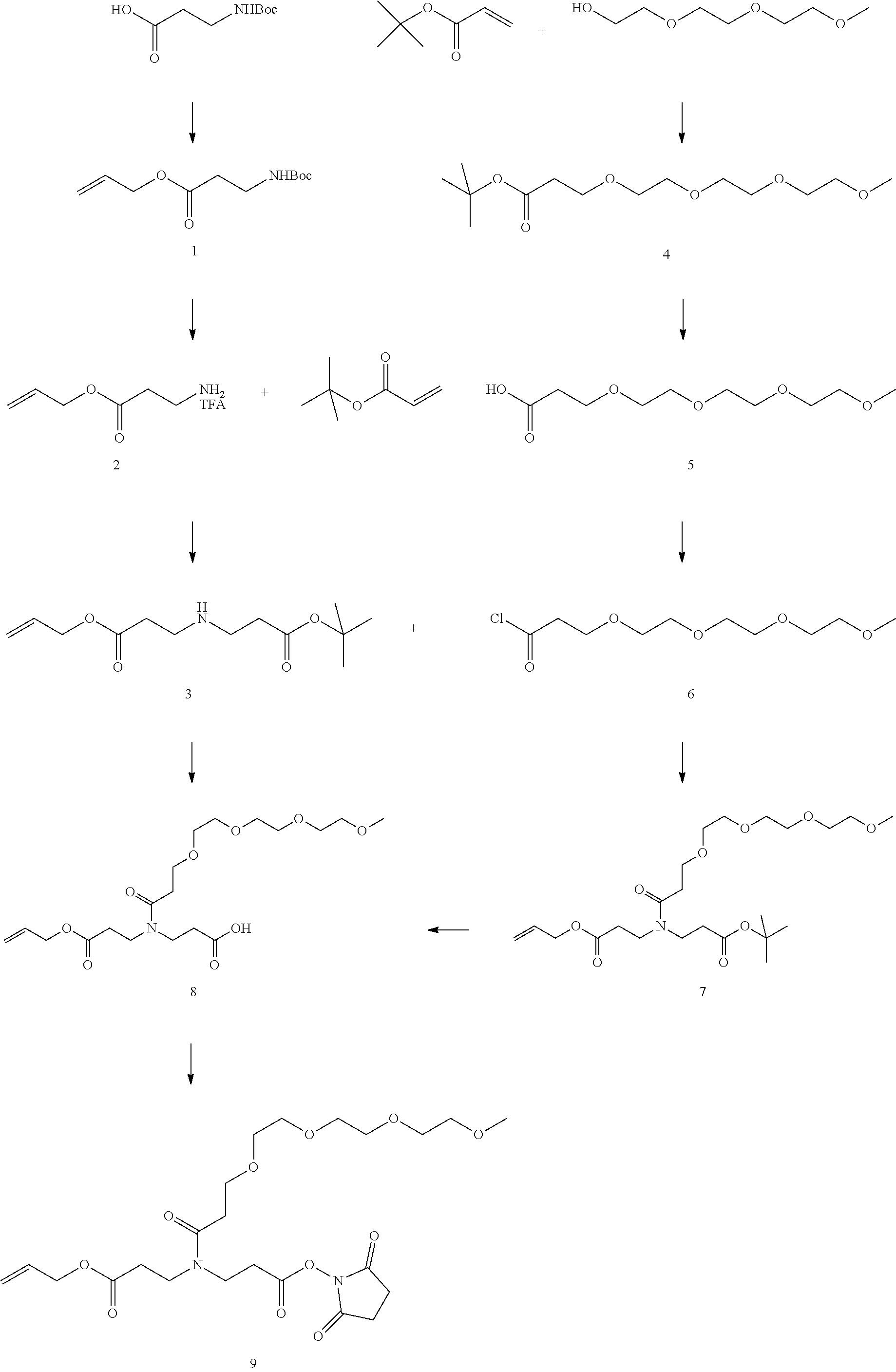

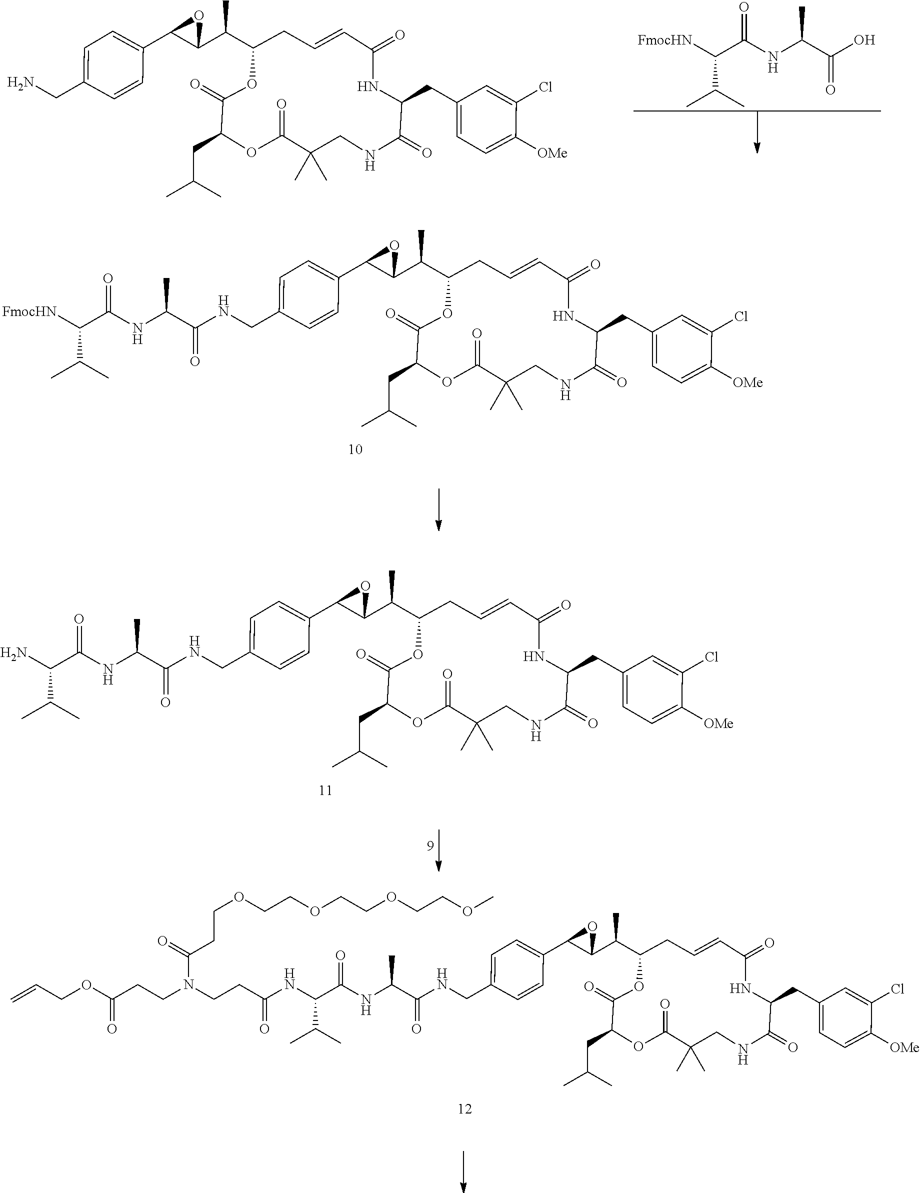

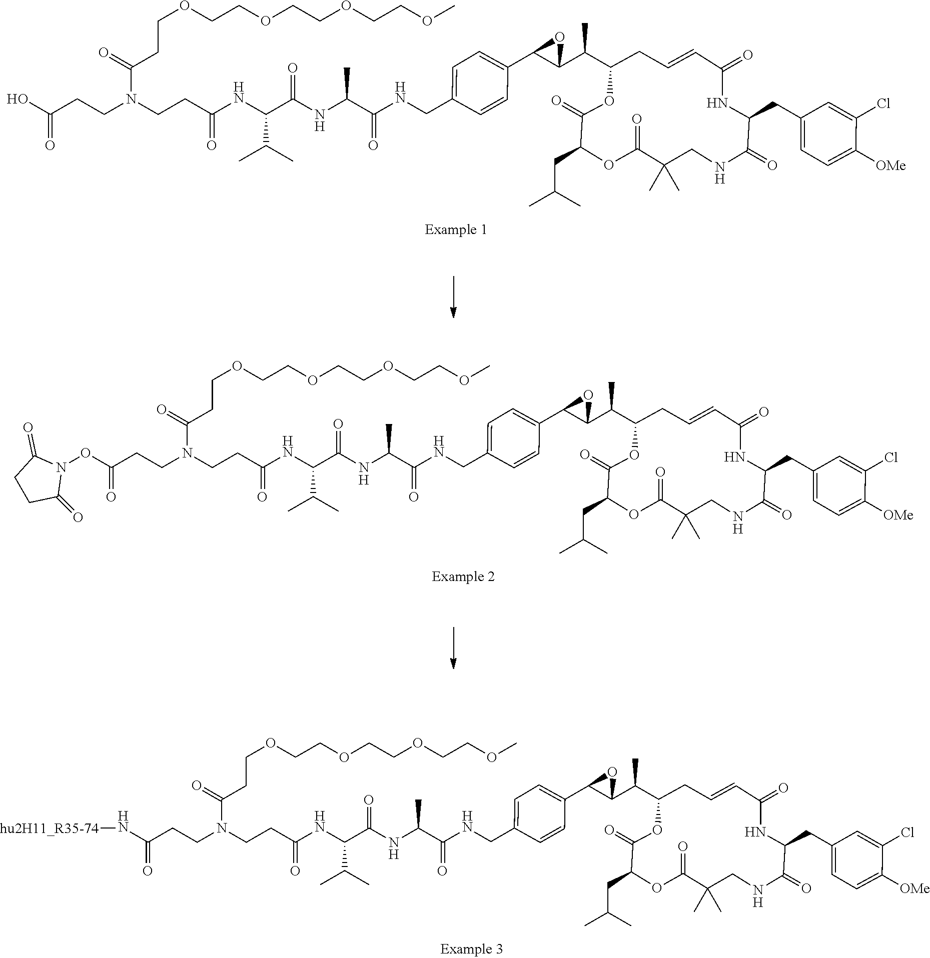

### Compound 1: allyl 3-((tert-butoxycarbonyl)amino)propanoate

To a solution of Boc-β-Ala-OH (CAS number [3303-84-2], 2 g, 10.04 mmol) in DMF (50 mL) were added cesium carbonate (6.88 g, 21.09 mmol) and allyl bromide (965.52 μL, 11.05 mmol). The reaction medium was stirred at RT overnight. At this time, the reaction medium was concentrated in vacuo, then diluted with toluene and concentrated in vacuo several times. The crude product was diluted with water (100 mL) and extracted with EtOAc (3×300 mL). The combined organic phases were washed with brine, dried over MgSO₄, filtered, concentrated in vacuo and purified by flash chromatography on 90 g of silica gel (gradient elution DCM/MeOH) to give 1.5 g of compound 1 (65%).

RMN ¹H (400 MHz, δ in ppm, DMSO-d6): 1.37 (s, 9H); 2.45 (t, J=6.9 Hz, 2H); 3.17 (q, J=6.9 Hz, 2H); 4.54 (m, 2H); 5.20 (qd, J=1.5 and 10.6 Hz, 1H); 5.29 (qd, J=1.9 and 17.3 Hz, 1H); 5.91 (m, 1H); 6.85 (broad t, J=6.9 Hz, 1H).

### Compound 2: allyl 3-aminopropanoate 2,2,2-trifluoroacetate

To a solution of compound 1 (1.5 g, 6.54 mmol) in DCM (30 mL) was added TFA (5.09 mL, 65.42 mmol). The reaction medium was stirred at RT overnight. At this time, the reaction medium was concentrated in vacuo, then diluted with toluene and concentrated in vacuo twice to afford 1.6 g of compound 2 (quant.).

### Compound 3: allyl 3-((3-(tert-butoxy)-3-oxopropyl)amino)propanoate

To a solution of compound 3 (1.6 g, 6.58 mmol) in DMSO (15 mL) were added DIEA (2.30 mL, 13.16 mmol) and tert-butyl acrylate (973.49 μL, 6.58 mmol). The reaction medium was heated 1 h at 60° C. using microwaves. At this time, the reaction medium was diluted with H₂O (10 mL) and extracted with EtOAc. The combined organic phases were washed with brine, dried over MgSO₄, filtered, concentrated in vacuo and purified by flash chromatography on 80 g of silica gel (gradient elution DCM/MeOH) to give 678 mg of compound 3 (40%).

RMN ¹H (400 MHz, δ in ppm, DMSO-d6): 1.39 (s, 9H); 1.73 (broad m, 1H); 2.29 (t, J=6.9 Hz, 2H); 2.44 (t, J=6.9 Hz, 2H); 2.68 (t, J=6.9 Hz, 2H); 2.73 (t, J=6.9 Hz, 2H); 4.53 (m, 2H); 5.20 (qd, J=1.5 and 10.6 Hz, 1H); 5.30 (qd, J=1.9 and 17.3 Hz, 1H); 5.91 (m, 1H).

### Compound 4: tert-butyl 2,5,8,11-tetraoxatetradecan-14-oate

Under argon, a mixture of triethylene glycol monomethyl ether (CAS number [112-35-6], 3 g, 17.36 mmol) and sodium (3.99 mg, 173.57 μmol) in THF (9 mL) was stirred for 2 h at 50° C. then overnight at RT. At this time, tert-butyl acrylate (3.08 ml, 20.83 mmol) was added, stirred for 2 h at 40° C. then overnight at RT. After that, the reaction medium was concentrated in vacuo, then diluted H₂O and extracted with EtOAc. The combined organic phases were washed with brine, dried over MgSO₄, filtered, concentrated in vacuo and purified by flash chromatography on 90 g of silica gel (gradient elution heptane/EtOAc) to give 3.3 g of compound 4 (65%).

### Compound 5: 2,5,8,11-tetraoxatetradecan-14-oic acid

To a solution of compound 4 (2.5 g, 8.55 mmol) in DCM (30 mL) was added TFA (6.65 mL, 85.51 mmol). The reaction medium was stirred overnight at RT. At this time, the reaction medium was concentrated in vacuo, then diluted with toluene and concentrated in vacuo three times to give 3 g of compound 5 (quant.).

### Compound 6: 2,5,8,11-tetraoxatetradecan-14-oyl chloride

To compound 5 (800 mg, 3.39 mmol) was added thionyl chloride (3.68 mL, 50.79 mmol). The reaction medium was heated 3 h at 60° C. After cooling, the reaction medium was concentrated in vacuo, diluted with DCM and concentrated in vacuo twice to give 870 mg of compound 6 (quant.).

### Compound 7: allyl 15-(3-(tert-butoxy)-3-oxopropyl)-14-oxo-2,5,8,11-tetraoxa-15-azaoctadecan-18-oate

At 0° C., under argon, to a solution of compound 3 (678 mg, 2.63 mmol) and DIEA (533.21 μL, 3.16 mmol) in DCM (10 mL) was added a solution of compound 6 (738.21 mg, 2.90 mmol) in DCM (3 mL). After 2 h, the reaction medium was washed with H₂O (10 mL) then brine, dried over MgSO₄, filtered, concentrated in vacuo and purified by flash chromatography on 40 g of silica gel (gradient elution DCM/MeOH) to give 410 mg of compound 7 (33%).

RMN ¹H (400 MHz, δ in ppm, DMSO-d6): 50/50 conformer mixture; 1.39 (s, 4.5H); 1.40 (s, 4.5H); 2.39 (t, J=7.2 Hz, 1H); 2.47 to 2.60 (partially masked m, 4H); 2.66 (t, J=7.2 Hz, 1H); 3.23 (s, 1.5H); 3.24 (s, 1.5H); 3.38 to 3.65 (m, 18H); 4.54 (m, 2H); 5.19 to 5.34 (m, 2H); 5.85 to 5.97 (m, 1H).

### Compound 8: 15-(3-(allyloxy)-3-oxopropyl)-14-oxo-2,5,8,11-tetraoxa-15-azaoctadecan-18-oic acid

A solution of compound 7 (150 mg, 315.41 μmol) in DCM (3 mL) and TFA (236.66 μL, 3.15 mmol) was stirred at RT overnight. At this time, the reaction medium was concentrated in vacuo, diluted with toluene and concentrated in vacuo three times to give 130 mg of compound 8 (98%).

### Compound 9: allyl 15-(3-((2,5-dioxopyrrolidin-1-yl)oxy)-3-oxopropyl)-14-oxo-2,5,8,11-tetraoxa-15-azaoctadecan-18-oate

To a solution of compound 8 (130 mg, 309.92 μmol) in THF (5 mL) were added DIEA (104.53 μL, 619.83 μmol) and DSC (97.21 mg, 371.90 μmol). The reaction medium was stirred at RT overnight. At this time, the reaction medium was concentrated in vacuo and purified by flash chromatography on 4 g of silica gel (gradient elution DCM/iPrOH) to give 107 mg of compound 9 (67%).

### Compound 10: (9H-fluoren-9-yl)methyl ((2S)-1-(((2S)-1-((4-((2R,3S)-3-(1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)amino)-1-oxopropan-2-yl)amino)-3-methyl-1-oxobutan-2-yl)carbamate

To a solution of (3S,10R,16S,E)-16-((S)-1-((2R,3R)-3-(4-(aminomethyl)phenyl)-oxiran-2-yl)ethyl)-10-(3-chloro-4-methoxybenzyl)-3-isobutyl-6,6-dimethyl-1,4-dioxa-8,11-diazacyclohexadec-13-ene-2,5,9,12-tetraone (the synthesis of which was described in WO2011001052, compound 77,150 mg, 214.82 μmol) in DCM was added HOBt (31.93 mg, 236.31 μmol), EDC (41.83 μL, 236.31 μmol) and Fmoc-Val-Ala-OH (CAS number [150114-97-9], 79.36 mg, 193.34 μmol). The reaction medium was stirred for 3 h at RT. Then H₂O (20 mL) was added and the mixture was extracted with DCM (2×15 mL). The combined organic phases were washed with brine, dried over MgSO₄, filtered, concentrated in vacuo and purified by flash chromatography on 5 g of silica gel (gradient elution DCM/MeOH) to give 200 mg of compound 10 (85%).

### Compound 11: (2S)-2-amino-N-((2S)-1-((4-((2R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)amino)-1-oxopropan-2-yl)-3-methylbutanamide

A mixture of compound 10 (200 mg, 183.37 μmol) in DCM (10 mL) and piperidine (183.39 μL, 1.83 mmol) was stirred for 2 h at RT. The reaction medium was concentrated in vacuo, solubilized in DCM before addition of Et₂O. The precipitate was filtered, washed with Et₂O and dried to give 180 mg of crude that was purified by flash chromatography on 10 g of amino-propyl modified silica gel (gradient elution DCM/MeOH) to give 136 mg of compound 11 (85%).

### Compound 12: allyl 15-(3-(((2S)-1-(((2S)-1-((4-((2R,3S)-3-(1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)amino)-1-oxopropan-2-yl)amino)-3-methyl-1-oxobutan-2-yl)amino)-3-oxopropyl)-14-oxo-2,5,8,11-tetraoxa-15-azaoctadecan-18-oate

To a solution of compound 11 (60 mg, 69.09 μmol) in THF (5 mL) were added compound 9 (42.82 mg, 82.91 μmol) and DIEA (17.48 μL, 103.63 μmol). The reaction medium was stirred for 3 h at RT. At this time, it was concentrated in vacuo and purified by flash chromatography on 10 g of silica gel (gradient elution DCM/MeOH) to give 60 mg of compound 12 (68%).

RMN ¹H (MHz, δ in ppm, DMSO-d6): 60/40 conformer mixture; 0.78 (d, J=7.0 Hz, 6H); 0.80 to 0.87 (m, 6H); 1.00 (s, 3H); 1.04 (d, J=7.0 Hz, 3H); 1.12 (s, 3H); 1.24 (split d, J=7.0 Hz, 3H); 1.30 (m, 1H); 1.51 to 1.63 (m, 2H); 1.75 to 1.83 (m, 1H); 1.95 (m, 1H); 2.26 (m, 1H); 2.30 to 2.73 (m, 8H); 2.95 to 3.05 (m, 3H); 3.23 (s, 3H); 3.31 (masked m, 1H); 3.40 to 3.64 (m, 18H); 3.81 (s, 3H); 3.87 (d, J=2.1 Hz, 1H); 4.12 to 4.33 (m, 5H); 4.53 (m, 1.2H); 4.55 (m, 0.8H); 4.91 (m, 1H); 5.11 (m, 1H); 5.21 (m, 1H); 5.28 (qd, J=1.9 and 17.3 Hz, 0.6H); 5.31 (qd, J=1.9 and 17.3 Hz, 0.4H); 5.78 (split d, J=15.7 Hz, 1H); 5.85 to 5.95 (m, 1H); 6.47 (ddd, J=4.2, 11.5 and 15.7 Hz, 1H); 7.05 (d, J=8.7 Hz, 1H); 7.17 (dd, J=2.3 and 8.7 Hz, 1H); 7.19 to 7.26 (m, 5H); 7.28 (d, J=2.3 Hz, 1H); 7.94 (d, J=9.2 Hz, 0.4H); 8.04 (m, 1H); 8.10 (d, J=7.6 Hz, 0.6H); 8.32 (m, 1H); 8.35 (d, J=8.2 Hz, 1H).

### Example 1: 15-(3-(((S)-1-(((S)-1-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)amino)-1-oxopropan-2-yl)amino)-3-methyl-1-oxobutan-2-yl)amino)-3-oxopropyl)-14-oxo-2,5,8,11-tetraoxa-15-azaoctadecan-18-oic acid

Under argon, to a solution of compound 12 (60 mg, 47.25 μmol) in DCM (5 mL) were added tetrakis(triphenylphosphine)palladium(0) (2.76 mg, 2.36 μmol) and 1,3-dimethylbarbituric acid (22.58 mg, 141.74 μmol). The reaction medium was stirred for 1 h at RT and concentrated in vacuo. The crude product was purified by flash chromatography on 4 g of silica gel (gradient elution DCM/MeOH) to give 31 mg of example 1 (53%).

RMN ¹H (500 MHz, δ in ppm, DMSO-d6): 0.79 (d, J=7.0 Hz, 6H); 0.80 to 0.88 (m, 6H); 1.00 (s, 3H); 1.04 (d, J=7.0 Hz, 3H); 1.12 (s, 3H); 1.23 (m, 3H); 1.31 (m, 1H); 1.51 to 1.62 (m, 2H); 1.80 (m, 1H); 1.95 (m, 1H); 2.27 (m, 1H); 2.35 to 2.60 (partially masked m, 6H); 2.62 to 2.73 (m, 2H); 2.93 to 3.06 (m, 3H); 3.23 (s, 3H); 3.28 to 3.35 (masked m, 1H); 3.36 to 3.65 (m, 18H); 3.81 (s, 3H); 3.87 (d, J=2.0 Hz, 1H); 4.11 to 4.34 (m, 5H); 4.91 (m, 1H); 5.10 (m, 1H); 5.79 (d, J=15.5 Hz, 1H); 6.48 (ddd, J=3.8, 11.3 and 15.5 Hz, 1H); 7.05 (d, J=8.7 Hz, 1H); 7.17 (dd, J=2.0 and 8.7 Hz, 1H); 7.24 (m, 5H); 7.29 (d, J=2.0 Hz, 1H); 7.95 to 8.18 (m, 2H); 8.30 to 8.46 (m, 2H); 12.27 (broad m, 1H). LCMS (A): ES m/z=615 [M+2H]²⁺; m/z=1227 [M−H]⁻; m/z=1229 [M+H]⁺; t_{R}=1.26 min.

### Example 2: 2,5-dioxopyrrolidin-1-yl 15-(3-(((S)-1-(((S)-1-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)amino)-1-oxopropan-2-yl)amino)-3-methyl-1-oxobutan-2-yl)amino)-3-oxopropyl)-14-oxo-2,5,8,11-tetraoxa-15-azaoctadecan-18-oate

To a solution of example 1 (30 mg, 24.39 μmol) in THF (5 mL) were added DSC (7.65 mg, 29.27 μmol) and DIEA (9.88 μL, 58.54 μmol). The reaction medium was stirred at RT overnight then concentrated in vacuo and purified by flash chromatography on 5 g of diol modified silica gel (gradient elution DCM/iPrOH) to give 17 mg of example 2 (53%).

RMN ¹H (500 MHz, δ in ppm, DMSO-d6): 70/30 conformer mixture; 0.79 (d, J=7.0 Hz, 6H); 0.80 to 0.87 (m, 6H); 1.00 (s, 3H); 1.04 (d, J=7.0 Hz, 3H); 1.12 (s, 3H); 1.21 to 1.34 (m, 4H); 1.51 to 1.62 (m, 2H); 1.80 (m, 1H); 1.93 (m, 1H); 2.27 (m, 1H); 2.35 to 2.72 (partially masked m, 6H); 2.81 (s large, 4H); 2.88 to 3.06 (m, 5H); 3.23 (s, 3H); 3.28 to 3.37 (masked m, 1H); 3.40 to 3.79 (m, 18H); 3.81 (s, 3H); 3.88 (s, 1H); 4.13 to 4.33 (m, 5H); 4.90 (m, 1H); 5.10 (m, 1H); 5.79 (d, J=15.5 Hz, 1H); 6.46 (ddd, J=3.8, 11.5 and 15.5 Hz, 1H); 7.05 (d, J=8.7 Hz, 1H); 7.17 (broad s, 1H); 7.24 (m, 5H); 7.29 (broad s, 1H); 7.94 (d, J=8.8 Hz, 0.3H); 8.06 (m, 1H); 8.12 (d, J=8.1 Hz, 0.7H); 8.32 (m, 1H); 8.36 (d, J=8.1 Hz, 1H). LCMS (A): ES m/z=663.5 [M+2H]²⁺; m/z=1324 [M−H]⁻; m/z=1326 [M+H]⁺; m/z=1374 [M−H+HCO₂H]⁻; t_{R}=1.3 min.

### Example 3: hu2H11_R35-74-Ex2

The general method described previously was used for the preparation of example 3. 15 mg of hu2H11_R35-74 were reacted with 55.2 μL of a 9.1 mM solution of example 2 in DMA (10 eq.) for 3 h 30. After purification on Superdex 200 μg in buffer B pH 6.5+10% NMP, concentration on Amicon Ultra-15, buffer exchange on Nap-10 in buffer B pH 6.5+5% NMP and filtration on 0.22 μm PVDF filter, 3.36 mg of example 3 were obtained as a colorless limpid solution at a concentration of 2.24 mg/mL with a DAR of 3.4 (HRMS), a monomeric purity of 99.8% and a global yield of 22%.

SEC-HRMS: m/z=149354 (naked mAb); m/z=150563 (D1); m/z=151774 (D2); m/z=152966 (D3); m/z=154169 (D4); m/z=155410 (D5); m/z=156623 (D6); m/z=157835 (D7); m/z=159051 (D8).

### Synthesis of Examples 4 to 6: sulfo-Val-Ala-C52 benzylic amine, NHS ester of sulfo-Val-Ala-C52 benzylic amine and Corresponding ADC

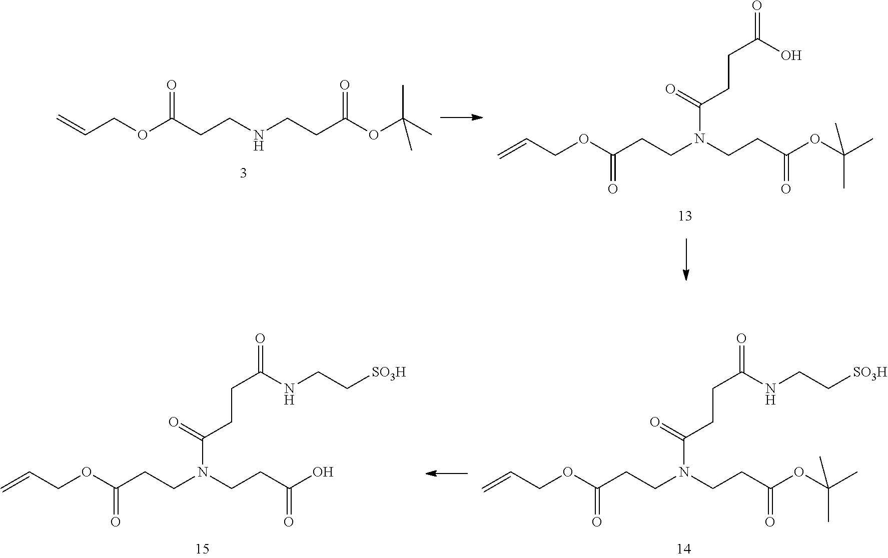 [SMILES: C=CCOC(=O)CCNCCC(=O)OC(C)(C)C ; C=CCOC(=O)CCN(CCC(=O)OC(C)(C)C)C(=O)CCC(=O)O ; C=CCOC(=O)CCN(CCC(=O)O)C(=O)CCC(=O)NCCS(=O)(=O)O ; C=CCOC(=O)CCN(CCC(=O)OC(C)(C)C)C(=O)CCC(=O)NCCS(=O)(=O)O]

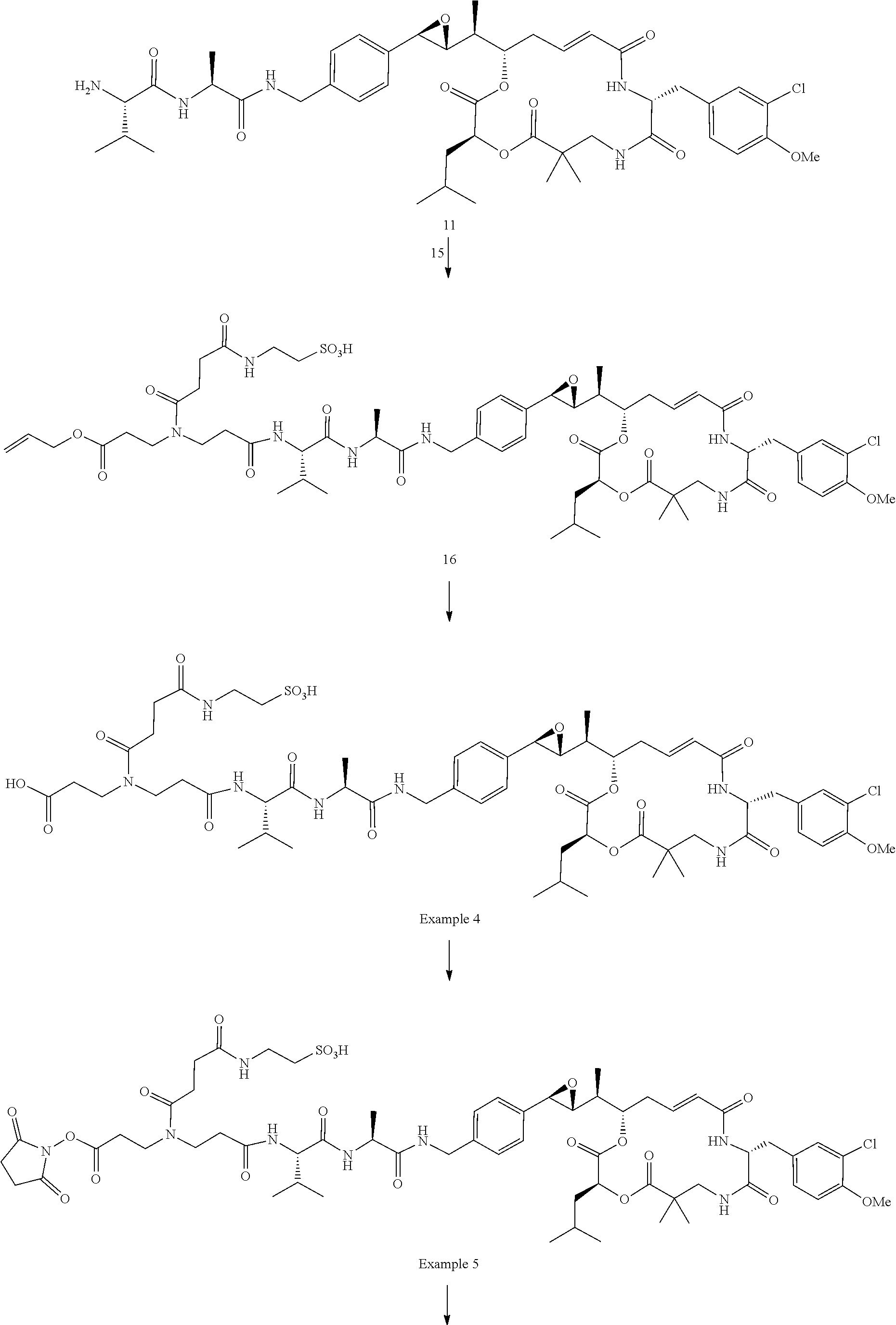

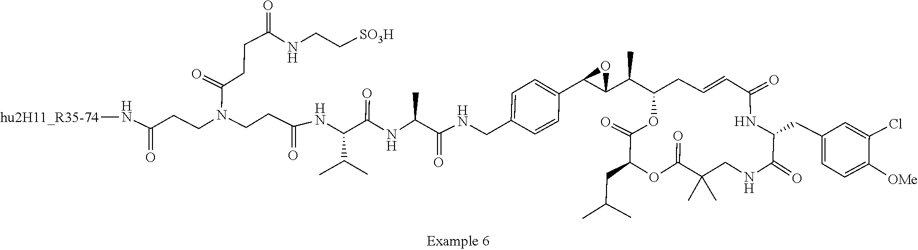

### Compound 13: 4-((3-(allyloxy)-3-oxopropyl)(3-(tert-butoxy)-3-oxopropyl)amino)-4-oxobutanoic acid

A solution of compound 3 (958 mg, 3.72 mmol) in DCM (5 mL) and succinic anhydride (752.65 mg, 7.45 mmol) was stirred for 3 h at RT. The reaction medium was concentrated in vacuo and purified by flash chromatography on 24 g of silica gel (gradient elution DCM/MeOH) to give 1.3 g of compound 13 (98%).

RMN ¹H (400 MHz, δ in ppm, DMSO-d6): 50/50 conformer mixture; 1.39 (s, 4.5H); 1.40 (s, 4.5H); 2.33 to 2.43 (m, 3H); 2.45 to 2.55 (partially masked m, 4H); 2.69 (t, J=7.5 Hz, 1H); 3.40 (t, J=7.5 Hz, 1H); 3.45 (t, J=7.5 Hz, 1H); 3.53 (t, J=7.5 Hz, 1H); 3.59 (t, J=7.5 Hz, 1H); 4.53 (m, 1H); 4.59 (m, 1H); 5.19 to 5.35 (m, 2H); 5.91 (m, 1H); 12.06 (broad m, 1H).

### Compound 14: 2-(4-((3-(allyloxy)-3-oxopropyl)(3-(tert-butoxy)-3-oxopropyl)amino)-4-oxobutanamido)ethanesulfonic acid

A mixture of compound 13 (1.3 g, 3.64 mmol) in THF (12 mL) and DSC (1.14 g, 4.36 mmol) and DIEA (1.84 mL, 10.91 mmol) was stirred for 5 h at RT. After this time, a solution of taurine (718.75 mg, 5.46 mmol) in water (9 mL) was added and the medium was stirred at RT overnight. At that time, DSC (1.14 g, 4.36 mmol) and DIEA (1.84 mL, 10.91 mmol) were added and the medium was stirred at RT overnight. At this time, taurine (2.4 g, 18.2 mmol) in water (5 mL) were added and the reaction medium was stirred for 1 h at RT then concentrated and purified by flash chromatography on 70 g of C18-grafted silica gel (gradient elution CH₃CN/H₂O) to give 1.28 g of compound 14 (76%).

RMN ¹H (400 MHz, δ in ppm, DMSO-d6): 50/50 conformer mixture; 1.39 (s, 4.5H); 1.40 (s, 4.5H); 2.26 (t, J=7.2 Hz, 2H); 2.38 (t, J=7.5 Hz, 1H); 2.47 to 2.55 (partially masked m, 6H); 2.69 (t, J=7.5 Hz, 1H); 3.28 (m, 2H); 3.40 (t, J=7.5 Hz, 1H); 3.44 (t, J=7.5 Hz, 1H); 3.52 (t, J=7.5 Hz, 1H); 3.58 (t, J=7.5 Hz, 1H); 4.53 (m, 1H); 4.58 (m, 1H); 5.19 to 5.35 (m, 2H); 5.91 (m, 1H); 7.70 (t large, J=6.5 Hz, 1H).

### Compound 15: 3-(N-(3-(allyloxy)-3-oxopropyl)-4-oxo-4-((2-sulfoethyl)-amino)butanamido)propanoic acid

A solution of compound 14 (300 mg, 645.81 μmol) in DCM (5 mL) and TFA (484.57 μL, 6.46 mmol) was stirred at RT overnight. At this time, TFA (250 μL) was added. The reaction medium was stirred several hours and concentrated in vacuo, then diluted with toluene and concentrated in vacuo twice to give 260 mg of compound 15 (99%).

RMN ¹H (400 MHz, δ in ppm, DMSO-d6): 50/50 conformer mixture; 2.27 (t, J=7.2 Hz, 2H); 2.40 (t, J=7.6 Hz, 1H); 2.48 to 2.56 (partially masked m, 6H); 2.68 (t, J=7.6 Hz, 1H); 3.28 (m, 2H); 3.40 (t, J=7.6 Hz, 1H); 3.46 (t, J=7.6 Hz, 1H); 3.50 (t, J=7.6 Hz, 1H); 3.59 (t, J=7.6 Hz, 1H); 4.54 (m, 1H); 4.57 (m, 1H); 5.18 to 5.35 (m, 2H); 5.91 (m, 1H); 7.65 (broad t, J=6.5 Hz, 1H). LCMS (D): ES m/z=409 [M+H]⁺; t_{R}=1.49 min.

### Compound 16: (4S,7S)-12-(3-(allyloxy)-3-oxopropyl)-1-(4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)phenyl)-7-isopropyl-4-methyl-3,6,9,13,16-pentaoxo-2,5,8,12,17-pentaazanonadecane-19-sulfonic acid

Under argon, to a solution of compound 15 (28.22 mg, 69.09 μmol) in DMF (3 mL) were added DIEA (23.30 μL, 138.18 μmol), HOBt (9.72 mg, 69.09 μmol), EDC (12.49 μL, 69.09 μmol) and a solution of compound 11 (60 mg, 69.09 μmol) in DMF (2 mL). The reaction medium was stirred at RT overnight. To complete the reaction, compound 15 (28.22 mg, 69.09 μmol), DIEA (23.30 μL, 138.18 μmol), HOBt (9.72 mg, 69.09 μmol), EDC (12.49 μL, 69.09 μmol) were added and the reaction medium was stirred at RT for 1 d. At this time, the reaction medium was concentrated in vacuo and purified by flash chromatography on 70 g of C18-grafted silica gel (gradient elution CH₃CN/H₂O) to give 24 mg of compound 16 (24%).

RMN ¹H (500 MHz, δ in ppm, DMSO-d6): 60/40 conformer mixture; 0.79 (d, J=7.0 Hz, 6H); 0.80 to 0.87 (m, 6H); 1.00 (s, 3H); 1.04 (d, J=7.0 Hz, 3H); 1.11 (s, 3H); 1.23 (d, J=7.0 Hz, 3H); 1.30 (m, 1H); 1.52 to 1.63 (m, 2H); 1.80 (m, 1H); 1.95 (m, 1H); 2.27 (m, 3H); 2.34 to 2.58 (partially masked m, 6H); 2.63 to 2.73 (m, 2H); 2.93 to 3.03 (m, 3H); 3.25 to 3.59 (partially masked m, 9H); 3.81 (s, 3H); 3.88 (d, J=1.4 Hz, 1H); 4.10 to 4.33 (m, 5H); 4.53 (m, 1.2H); 4.56 (m, 0.8H); 4.91 (m, 1H); 5.10 (m, 1H); 5.20 (m, 1H); 5.29 (qd, J=1.8 and 17.3 Hz, 0.6H); 5.31 (qd, J=1.8 and 17.3 Hz, 0.4H); 5.79 (dd, J=1.8 and 15.4 Hz, 1H); 5.90 (m, 1H); 6.47 (ddd, J=3.8, 11.8 and 15.4 Hz, 1H); 7.05 (d, J=8.7 Hz, 1H); 7.18 (dd, J=2.4 and 8.7 Hz, 1H); 7.24 (m, 5H); 7.29 (d, J=2.4 Hz, 1H); 7.69 (t, J=5.8 Hz, 1H); 7.97 (d, J=8.8 Hz, 0.4H); 8.04 (d, J=7.7 Hz, 0.4H); 8.07 (d, J=8.8 Hz, 0.6H); 8.11 (d, J=7.7 Hz, 0.6H); 8.31 (t, J=6.3 Hz, 1H); 8.38 (d, J=8.3 Hz, 1H); 11.83 (broad m, 1H). LCMS (D): ES m/z=1256 [M−H]⁻; t_{R}=2.58 min.

### Example 4: (4S,7S)-1-(4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)phenyl)-7-isopropyl-4-methyl-3,6,9-trioxo-12-(4-oxo-4-((2-sulfoethyl)amino)butanoyl)-2,5,8,12-tetraazapentadecan-15-oic acid

Under argon, to a solution of compound 16 (24 mg, 19.06 μmol) in CH₃CN (2 mL) were added DMF (0.5 mL), 1,3-dimethylbarbituric acid (9.11 mg, 57.19 μmol) and tetrakis(triphenylphosphine)palladium(0) (1.11 mg, 0.953 μmol). The reaction medium was stirred for 1 h at RT, then concentrated in vacuo and purified by flash chromatography on 10 g of C18-grafted silica gel (gradient elution CH₃CN/H₂O) to give 16 mg of example 4 (70%).

RMN ¹H (500 MHz, δ in ppm, DMSO-d6): 60/40 conformer mixture; 0.79 (d, J=7.0 Hz, 6H); 0.80 to 0.86 (m, 6H); 0.99 (s, 3H); 1.04 (d, J=7.0 Hz, 3H); 1.12 (s, 3H); 1.24 (d, J=7.0 Hz, 3H); 1.31 (m, 1H); 1.52 to 1.62 (m, 2H); 1.80 (m, 1H); 1.95 (m, 1H); 2.21 to 2.31 (m, 3H); 2.34 to 2.59 (partially masked m, 8H); 2.62 to 2.75 (m, 2H); 2.94 to 3.05 (m, 3H); 3.25 to 3.58 (partially masked m, 7H); 3.81 (s, 3H); 3.88 (d, J=1.9 Hz, 1H); 4.09 to 4.33 (m, 5H); 4.90 (m, 1H); 5.10 (m, 1H); 5.79 (d, J=15.5 Hz, 1H); 6.47 (ddd, J=4.0, 11.4 and 15.5 Hz, 1H); 7.05 (d, J=8.7 Hz, 1H); 7.17 (dd, J=2.2 and 8.7 Hz, 1H); 7.21 to 7.26 (m, 5H); 7.29 (d, J=2.2 Hz, 1H); 7.69 (m, 1H); 7.97 (d, J=9.0 Hz, 0.4H); 8.04 (d, J=7.7 Hz, 0.4H); 8.08 (d, J=9.0 Hz, 0.6H); 8.12 (d, J=7.7 Hz, 0.6H); 8.31 (t, J=6.5 Hz, 1H); 8.38 (d, J=8.2 Hz, 1H); 12.2 (broad m, 1H). LCMS (A): ES m/z=609.5 [M+2H]²⁺; m/z=1216 [M−H]⁻; m/z=1218 [M+H]⁺; t_{R}=1.72 min.

### Example 5: (4S,7S)-1-(4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)phenyl)-12-(3-((2,5-dioxopyrrolidin-1-yl)oxy)-3-oxopropyl)-7-isopropyl-4-methyl-3,6,9,13,16-pentaoxo-2,5,8,12,17-pentaazanonadecane-19-sulfonic acid

Under argon, a mixture of example 4 (16 mg, 13.13 μmol) in DMF (1 mL), DSC (4.12 mg, 15.75 μmol) and DIEA (2.66 μL, 15.75 μmol) was stirred at RT overnight. At this time, the reaction medium was concentrated in vacuo and purified by flash chromatography on 5 g of C18-grafted silica gel (gradient elution CH₃CN/H₂O) to give 12.5 mg of example 5 (73%).

RMN ¹H (500 MHz, δ in ppm, DMSO-d6): 60/40 conformer mixture; 0.79 (d, J=7.0 Hz, 6H); 0.80 to 0.86 (m, 6H); 0.99 (s, 3H); 1.04 (d, J=7.0 Hz, 3H); 1.11 (s, 3H); 1.23 (d, J=7.0 Hz, 3H); 1.31 (m, 1H); 1.51 to 1.62 (m, 2H); 1.80 (m, 1H); 1.93 (m, 1H); 2.21 to 2.31 (m, 3H); 2.35 to 2.59 (partially masked m, 6H); 2.62 to 2.74 (m, 2H); 2.81 (broad s, 4H); 2.85 to 3.05 (m, 4H); 3.20 to 3.68 (partially masked m, 8H); 3.81 (s, 3H); 3.88 (d, J=1.9 Hz, 1H); 4.10 to 4.33 (m, 5H); 4.90 (m, 1H); 5.10 (m, 1H); 5.79 (d, J=15.5 Hz, 1H); 6.46 (ddd, J=4.0, 11.4 and 15.5 Hz, 1H); 7.05 (d, J=8.7 Hz, 1H); 7.18 (dd, J=2.2 and 8.7 Hz, 1H); 7.21 to 7.26 (m, 5H); 7.29 (d, J=2.2 Hz, 1H); 7.69 (m, 1H); 7.97 (d, J=9.0 Hz, 0.4H); 8.05 (d, J=7.7 Hz, 0.4H); 8.08 (d, J=9.0 Hz, 0.6H); 8.12 (d, J=7.7 Hz, 0.6H); 8.31 (t, J=6.5 Hz, 1H); 8.38 (d, J=8.2 Hz, 1H). LCMS (A): ES m/z=658 [M+2H]²⁺; m/z=1313 [M−H]⁻; m/z=1315 [M+H]⁺; t_{R}=1.82 min.

### Example 6: hu2H11_R35-74-Ex5

The general method described previously was used for the preparation of example 6. 15 mg of hu2H11_R35-74 were reacted with 42 μL of a 10 mM solution of example 5 in DMA (4 eq.) for 1 h 20 then were successively added 42 μL of a 10 mM solution of example 5 in DMA (4 eq.) for 1 h 20, 53 μL of a 10 mM solution of example 5 in DMA (5 eq.) for 1 h 30 and finally 42 μL of a 10 mM solution of example 5 in DMA (4 eq.) for 1 h. After purification on Superdex 200 μg in buffer B pH 6.5+20% NMP, concentration on Amicon Ultra-15, buffer exchange on PD-10 in buffer B pH 6.5+5% NMP and filtration on 0.22 μm PVDF filter, 3.33 mg of example 6 were obtained as a colorless limpid solution at a concentration of 1.33 mg/mL with a DAR of 4.05 (HRMS), a monomeric purity of 99% and a global yield of 22%.

SEC-HRMS: m/z=150568 (D1); m/z=151768 (D2); m/z=152971 (D3); m/z=154172 (D4); m/z=155375 (D5); m/z=156574 (D6); m/z=157944 (D7); m/z=158984 (D8).

### Synthesis of Example 7: sulfo-Val-Ala-aza-crypto benzylic amine

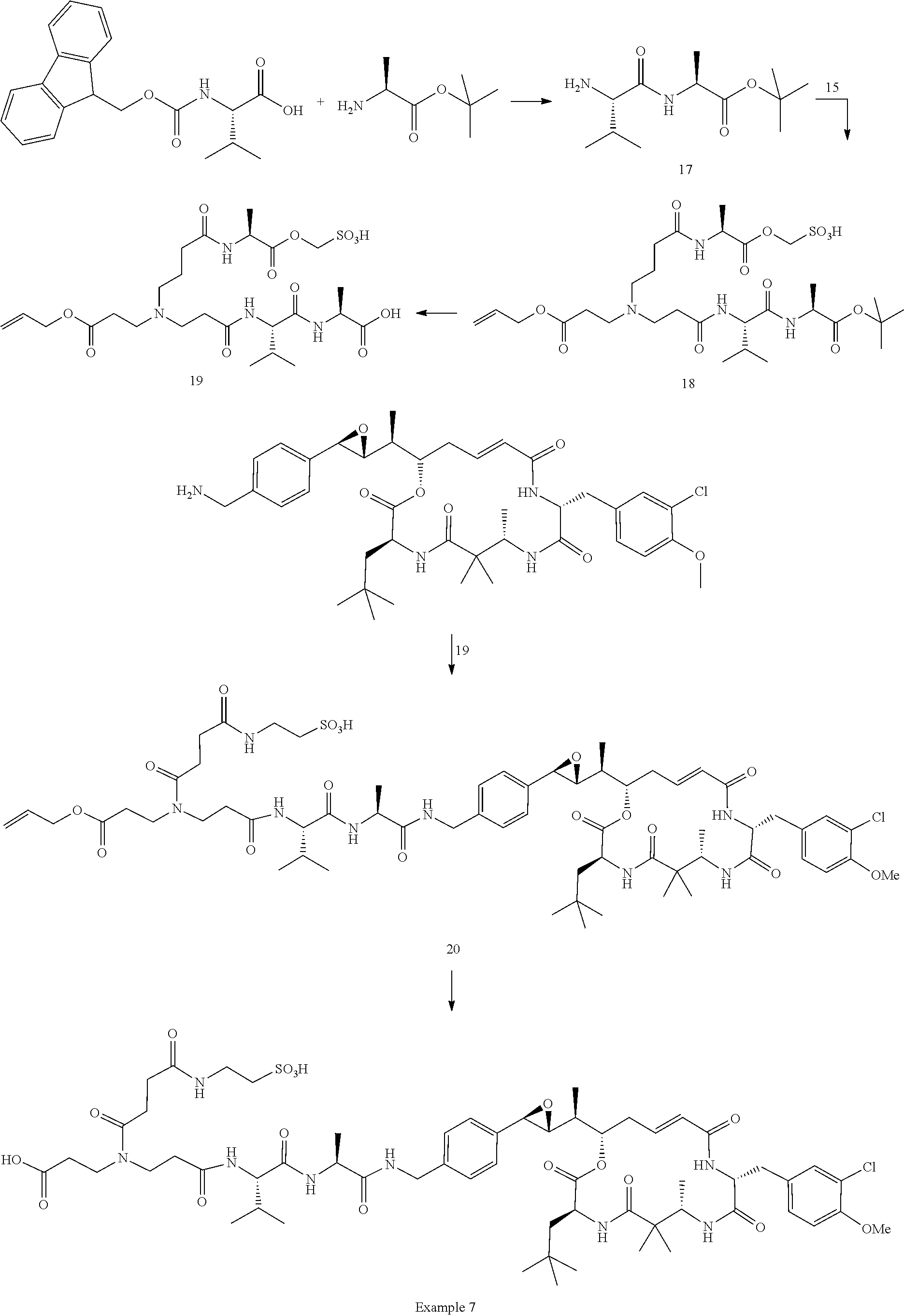

### Compound 17: tert-butyl L-valyl-L-alaninate

To a solution of Fmoc-Val-OH (CAS number [68858-20-8], 3 g, 8.66 mmol) in DCM (60 mL) were added, under Ar, EDC (2.02 g, 12.99 mmol), HOBt (1.46 g, 10.40 mmol) and L-alanine tert-butyl ester hydrochloride (CAS number [13404-22-3], 1.61 g, 8.66 mmol). The reaction medium was stirred for 1 d at RT, quenched with H₂O (30 mL) and extracted with DCM (3×30 mL). The combined organic phases were washed with brine, dried over MgSO₄, filtered, concentrated in vacuo to give 5 g of Fmoc-protected dipeptide as a white solid (quant.). To a solution of this intermediate (5 g, 8.66 mmol) in DCM (50 mL) was added piperidine (8.64 mL, 86.63 mmol). The reaction medium was stirred for 1 h at RT and concentrated in vacuo. The crude product was diluted with Et₂O and filtered to give 1.77 g of a solid that was further purified by two consecutive flash chromatographies on 120 g and 50 g of silica gel (gradient elution DCM/MeOH) to give 500 mg of compound 17 as a white solid (24%).

RMN ¹H (400 MHz, δ in ppm, DMSO-d6): 0.79 (d, J=6.9 Hz, 3H); 0.89 (d, J=6.9 Hz, 3H); 1.24 (d, J=7.3 Hz, 3H); 1.40 (s, 9H); 1.62 (broad s, 2H); 1.86 (m, 1H); 2.95 (d, J=5.2 Hz, 1H); 4.15 (m, 1H); 8.09 (broad d, J=7.3 Hz, 1H).

### Compound 18: (5S,8S)-13-(3-(allyloxy)-3-oxopropyl)-8-isopropyl-2,2,5-trimethyl-4,7,10,14,17-pentaoxo-3-oxa-6,9,13,18-tetraazaicosane-20-sulfonic acid

To a solution of compound 15 (250 mg, 612.1 μmol) in DMF (6 mL) were added, under Ar, DIEA (309.7 μL, 1.84 mmol) and DSC (163.34 mg, 612.1 μmol). The reaction medium was stirred for 1 h at RT then was added a solution of compound 17 (149.56 mg, 612.1 μL) in DMF (1 mL). The reaction medium was stirred for 2 h at RT and purified by reverse phase chromatography on a 5 μm C18 column 30×100 mm (gradient elution MeCN+0.07% TFA/H₂O+0.07% TFA) to give 215 mg of compound 18 as a white foam (55%).

RMN ¹H (400 MHz, δ in ppm, DMSO-d6): 60/40 conformer mixture; 0.84 (d, J=6.9 Hz 3H); 0.87 (d, J=6.9 Hz, 3H); 1.12 (d, J=7.3 Hz, 3H); 1.39 (s, 9H); 1.92 (m, 1H); 2.20 to 2.70 (partially masked m, 10H); 3.28 (m, 2H); 3.34 to 3.60 (m, 4H); 4.10 (m, 1H); 4.20 (m, 1H); 4.53 (td, J=1.6 and 5.5 Hz, 1.2H); 4.57 (td, J=1.6 and 5.5 Hz, 0.8H); 5.20 (m, 1H); 5.30 (m, 1H); 5.90 (m, 1H); 7.69 (broad t, J=6.4 Hz, 1H); 7.91 (d, J=9.6 Hz, 0.4H); 8.04 (d, J=9.6 Hz, 0.6H); 8.23 (d, J=7.2 Hz, 0.4H); 8.30 (d, J=7.2 Hz, 0.6H).

### Compound 19: (3-(N-(3-(allyloxy)-3-oxopropyl)-4-oxo-4-((2-sulfoethyl)amino)-butanamido)-propanoyl)-L-valyl-L-alanine

To a solution of compound 18 (215 mg, 338.72 μmol) in DCM (5 mL) was added TFA (508.3 μL, 6.77 mmol). The reaction medium was stirred for 1 d at RT, concentrated in vacuo and co-evaporated with toluene (3×) to give 200 mg of compound 19 as a white foam (quant.).

### Compound 20: (4S,7S)-12-(3-(allyloxy)-3-oxopropyl)-1-(4-((2R,3R)-3-((S)-1-((3S,7S,10R,16S,E)-10-(3-chloro-4-methoxybenzyl)-6,6,7-trimethyl-3-neopentyl-2,5,9,12-tetraoxo-1-oxa-4,8,11-triazacyclohexadec-13-en-16-yl)ethyl)oxiran-2-yl)phenyl)-7-isopropyl-4-methyl-3,6,9,13,16-pentaoxo-2,5,8,12,17-pentaazanona-decane-19-sulfonic acid

To a solution of compound 19 (200 mg, 345.6 μmol) in DMF (9 mL), were added DSC (108 mg, 413.2 μmol) and DIEA (200 μL, 1.185 mmol). The reaction medium was stirred for 6 h at RT then were added added DSC (108 mg, 413.2 μmol) and DIEA (200 μL, 1.185 mmol). The reaction medium was stirred at RT overnight then were added DSC (50 mg, 191.3 μmol) and DIEA (100 μL, 592.5 μmol) and stirring carried on for 1 h at RT. To 3 mL of the solution of activated ester were added (3S,7S,10R,16S,E)-16—((S)-1-((2R,3R)-3-(4-(aminomethyl)phenyl)oxiran-2-yl)ethyl)-10-(3-chloro-4-methoxybenzyl)-6,6,7-trimethyl-3-neopentyl-1-oxa-4,8,11-triazacyclo-hexadec-13-ene-2,5,9,12-tetraone (that can be synthesized as described in PCT/EP2016/076603 starting from methyl (3S)-3-amino-2,2-dimethylbutanoate, [MFCD09256689], 70 mg, 96.51 μmol), EDC (17 μL, 77.8 μmol) and HOBt (19 mg, 124 μmol). The reaction medium was stirred for 1 h at RT, concentrated in vacuo and purified by flash chromatography on 10 g of C18—modified silica gel (gradient elution MeCN/H₂O) to give 30 mg of compound 20 as a white lacquer (24%).

RMN ¹H (600 MHz, δ in ppm, DMSO-d6): 0.79 to 0.90 (m, 15H); 0.98 (m, 3H); 1.00 (s, 3H); 1.03 (d, J=7.3 Hz, 3H); 1.19 (s, 3H); 1.24 (split d, J=7.0 Hz, 3H); 1.34 (d, J=14.1 Hz, 1H); 1.81 (m, 1H); 1.91 (m, 1H); 1.98 (dd, J=9.3 and 14.1 Hz, 1H); 2.25 (m, 3H); 2.38 (m, 1H); 2.50 (partially masked m, 5H); 2.60 (m, 1H); 2.68 (m, 1H); 2.91 (m, 1H); 2.96 (m, 2H); 3.25 to 3.58 (partially masked m, 9H); 3.80 (s, 3H); 3.91 (m, 1H); 4.00 to 4.35 (m, 6H); 4.53 (m, 2H); 5.03 (m, 1H); 5.20 (m, 1H); 5.30 (m, 1H); 5.89 (d, J=16.1 Hz, 1H); 5.91 (m, 1H); 6.44 (ddd, J=5.0, 10.5 and 16.1 Hz, 1H); 7.03 (d, J=8.7 Hz, 1H); 7.19 to 7.28 (m, 5H); 7.31 (s, 1H); 7.69 (m, 1H); 7.86 (d, J=8.2 Hz, 1H); 7.91 (m, 1H); 7.95 to 8.17 (m, 2H); 8.32 (m, 1H); 8.39 (m, 1H). LCMS (A): ES m/z=1283 [M−H]⁻; m/z=1285 [M+H]⁺; m/z=1307 [M+Na]⁺; t_{R}=1.93 min.

### Example 7: (4S,7S)-1-(4-((2R,3R)-3-((S)-1-((3S,7S,10R,16S,E)-10-(3-chloro-4-methoxybenzyl)-6,6,7-trimethyl-3-neopentyl-2,5,9,12-tetraoxo-1-oxa-4,8,11-triazacyclohexadec-13-en-16-yl)ethyl)-oxiran-2-yl)phenyl)-7-isopropyl-4-methyl-3,6,9-trioxo-12-(4-oxo-4-((2-sulfoethyl)amino)butanoyl)-2,5,8,12-tetraazapentadecan-15-oic acid

To a solution of compound 20 (30 mg, 23.33 μmol) in DCM (2 mL) and DMF (0.3 mL) were added, under Ar, tetrakis(triphenylphosphine)palladium(0) (1.38 mg, 1.17 μmol) and 1,3-dimethyl-barbituric acid (11.15 mg, 69.99 μmol). The reaction medium was stirred for 1 h at RT and purified by reverse phase chromatography on 1 g of C18-modified silica gel (gradient elution MeCN/H₂O) to give 15 mg of a white powder that was further purified on silica gel (gradient elution DCM/MeOH/H₂O) to give 7 mg of example 7 as a white lacquer (24%).

### Synthesis of Examples 8 & 9: PEG4-Val-Ala-PABA-C52 benzylic amine and NHS ester of PEG4-Val-Ala-PABA-C52 benzylic amine

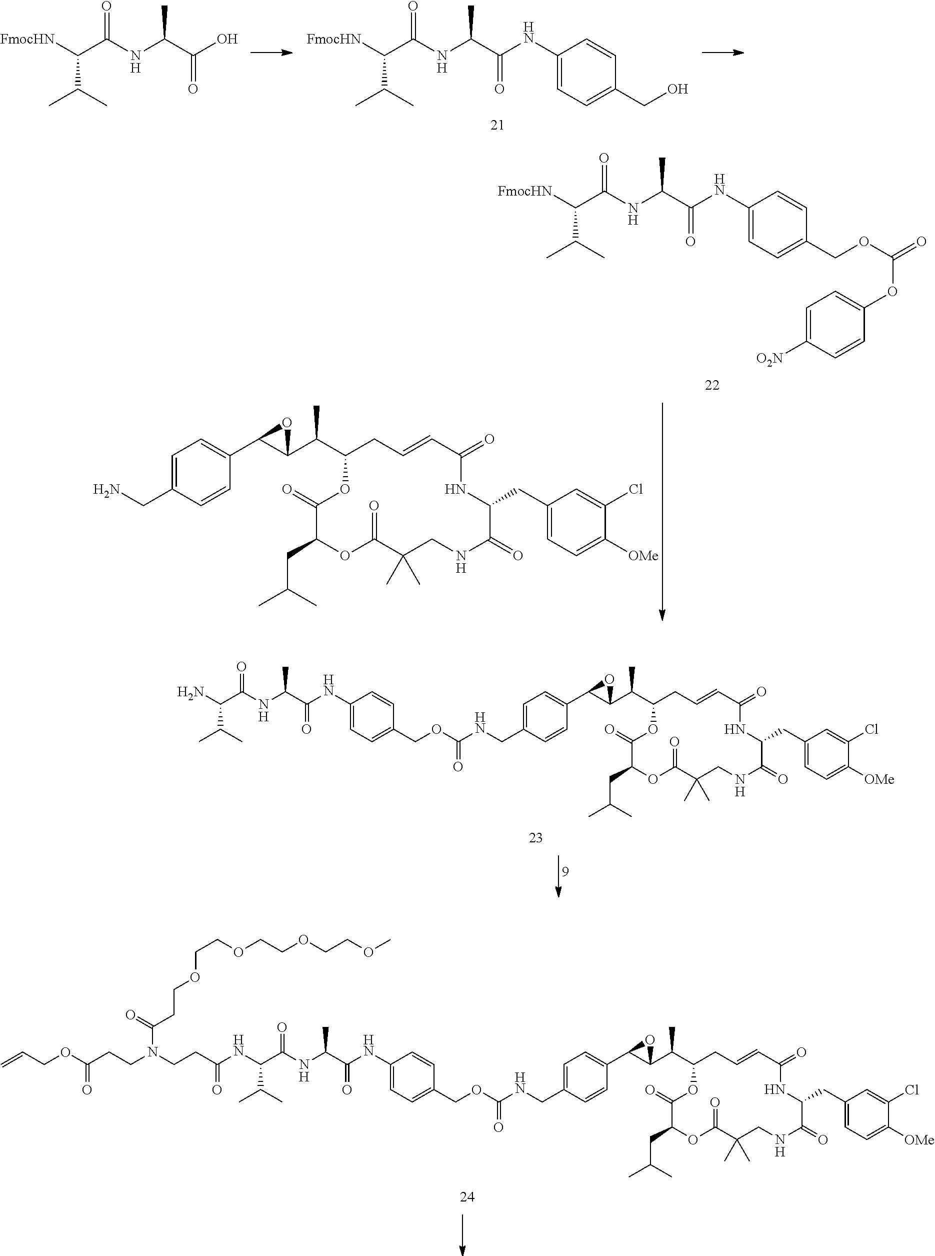

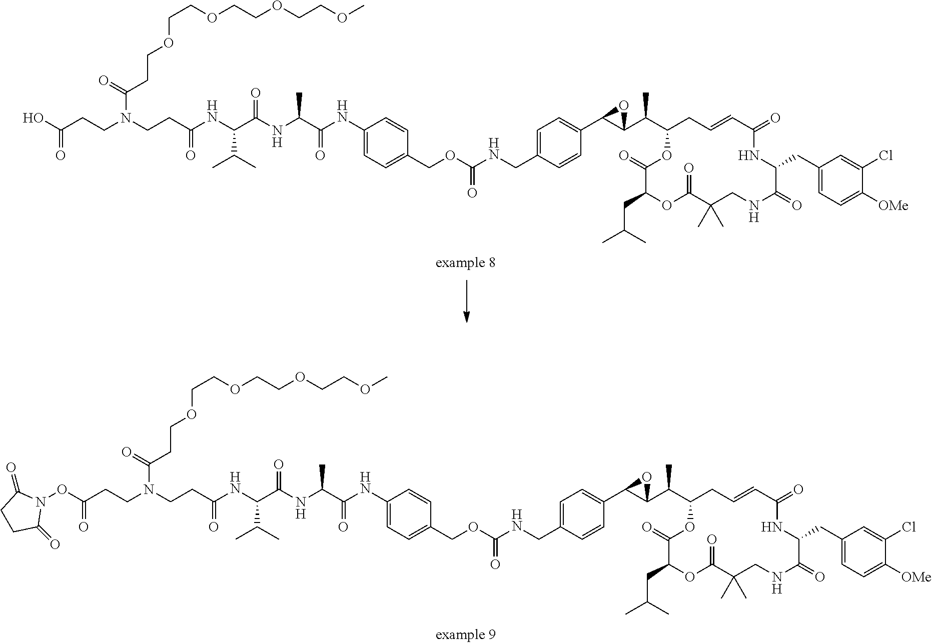 [SMILES: COCCOCCOCCOCCC(=O)N(CCC(=O)O)CCC(=O)N[C@H](C(=O)N[C@@H](C)C(=O)Nc1ccc(COC(=O)NCc2ccc([C@H]3O[C@@H]3[C@@H](C)[C@@H]3C/C=C/C(=O)N[C@H](Cc4ccc(OC)c(Cl)c4)C(=O)NCC(C)(C)C(=O)O[C@@H](CC(C)C)C(=O)O3)cc2)cc1)C(C)C ; COCCOCCOCCOCCC(=O)N(CCC(=O)N[C@H](C(=O)N[C@@H](C)C(=O)Nc1ccc(COC(=O)NCc2ccc([C@H]3O[C@@H]3[C@@H](C)[C@@H]3C/C=C/C(=O)N[C@H](Cc4ccc(OC)c(Cl)c4)C(=O)NCC(C)(C)C(=O)O[C@@H](CC(C)C)C(=O)O3)cc2)cc1)C(C)C)CCC(=O)ON1C(=O)CCC1=O]

### Compound 21: (9H-fluoren-9-yl)methyl ((S)-1-(((S)-1-((4-(hydroxymethyl)phenyl)-amino)-1-oxopropan-2-yl)amino)-3-methyl-1-oxobutan-2-yl)carbamate

To a solution of Fmoc-Val-Ala-OH (CAS number [150114-97-9], 1 g, 2.44 mmol) in DCM (30 mL) and MeOH (15 mL) were added 4-aminobenzyl alcohol (612 mg, 4.87 mmol) and EEDQ (1.22 g, 4.87 mmol). The reaction medium was stirred at RT for 24 h then additional 4-aminobenzyl alcohol (612 mg, 4.87 mmol) and EEDQ (1.22 g, 4.87 mmol) were added and the stirring carried on 2 h. The reaction medium was then concentrated in vacuo; Et₂O was added to the crude product, the mixture was stirred and filtered to give a brown solid that was diluted in Et₂O and the mixture was stirred at RT overnight. It was then filtered and the precipitate was dried to give 1.3 g of compound 21 as a brown solid (quant.).

RMN ¹H (400 MHz, δ in ppm, DMSO-d6): 0.86 (d, J=7.0 Hz, 3H); 0.89 (d, J=7.0 Hz, 3H); 1.30 (d, J=7.0 Hz, 3H); 2.00 (m, 1H); 3.91 (m, 1H); 4.19 to 4.34 (m, 4H); 4.43 (d, J=5.7 Hz, 2H); 5.08 (t, J=5.7 Hz, 1H); 7.23 (d, J=7.9 Hz, 2H); 7.31 (t, J=7.9 Hz, 2H); 7.40 (m, 3H); 7.53 (d, J=9.1 Hz, 2H); 7.73 (t, J=8.6 Hz, 2H); 7.89 (d, J=7.9 Hz, 2H); 8.16 (d, J=7.3 Hz, 1H); 9.91 (s, 1H). LCMS (A): ES m/z=292; m/z=393; m/z=516 [M+H]⁺; t_{R}=1.21 min.

### Compound 22: (9H-fluoren-9-yl)methyl ((S)-3-methyl-1-(((S)-1-((4-((((4-nitrophenoxy)carbonyl)-oxy)methyl)phenyl)amino)-1-oxopropan-2-yl)amino)-1-oxobutan-2-yl)carbamate

To a solution of compound 21 (1.3 g, 2.52 mmol) in DMF (10 mL), was added, under Ar, DIEA (2.55 mL, 15.13 mmol), the solution was cooled down at 0° C. before the addition of 4-nitrophenyl chloroformate (1.57 g, 7.56 mmol). The reaction medium was stirred for 2 h at RT. 20 mL of H₂O were added and the medium was extracted twice with EtOAc (10 mL). The combined organic phases were washed with brine, dried over MgSO₄, filtered, concentrated in vacuo and purified by flash chromatography on 120 g of silica gel (gradient elution DCM/MeOH) to give 600 mg of compound 22 as a solid (35%).

RMN ¹H (400 MHz, δ in ppm, DMSO-d6): 0.86 (d, J=7.0 Hz, 3H); 0.89 (d, J=7.0 Hz, 3H); 1.31 (d, J=7.0 Hz, 3H); 1.99 (m, 1H); 3.91 (m, 1H); 4.18 to 4.35 (m, 3H); 4.43 (m, 1H); 5.24 (s, 2H); 7.31 (t, J=7.9 Hz, 2H); 7.35 to 7.44 (m, 5H); 7.56 (d, J=9.1 Hz, 2H); 7.63 (d, J=8.6 Hz, 2H); 7.74 (m, 2H); 7.88 (d, J=7.9 Hz, 2H); 8.19 (d, J=7.3 Hz, 1H); 8.31 (d, J=9.1 Hz, 2H); 10.07 (s, 1H).

### Compound 23: 4-((S)-2-((S)-2-amino-3-methylbutanamido)propanamido)benzyl 4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzylcarbamate

To a solution of (3S,10R,16S,E)-16-((S)-1-((2R,3R)-3-(4-(aminomethyl)phenyl)oxiran-2-yl)ethyl)-10-(3-chloro-4-methoxybenzyl)-3-isobutyl-6,6-dimethyl-1,4-dioxa-8,11-diazacyclohexadec-13-ene-2,5,9,12-tetraone (the synthesis of which was described in WO2011001052, compound 77, 100 mg, 143.2 μmol) in THF (5 mL), were added, under argon compound 22 (117 mg, 171.9 μmol) and DIEA (60.4 μL, 358 μmol). The reaction medium was stirred at RT under Ar overnight. Then piperidine (142.9 μL, 1.43 mmol) was added and the reaction medium stirred for 1 h at RT. After concentrating in vacuo, the crude medium was purified by flash chromatography on 5 g of silica gel (gradient elution DCM/MeOH) to give 100 mg of compound 23 as a white solid (73%).

### Compound 24: allyl 15-(3-(((S)-1-(((S)-1-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclo-hexadec-13-en-16-yl)ethyl)oxiran-2-yl)benzyl)carbamoyl)oxy)-methyl)phenyl)amino)-1-oxopropan-2-yl)amino)-3-methyl-1-oxobutan-2-yl)amino)-3-oxopropyl)-14-oxo-2,5,8,11-tetraoxa-15-azaoctadecan-18-oate

To a solution of compound 23 (100 mg, 98.3 μmol) in THF (5 mL), were added, under Ar, compound 9 (50.8 mg, 98.3 μmol) and DIEA (24.9 μL, 147.4 μmol). The reaction medium was stirred for 1 h at RT, concentrated in vacuo and purified by flash chromatography on 5 g of silica gel (gradient elution DCM/MeOH) to give 128 mg of compound 24 as a white solid (91%).

RMN ¹H (500 MHz, δ in ppm, DMSO-d6): 60:40 conformer mixture; 0.78 (d, J=6.8 Hz, 6H); 0.82 (d, J=7.0 Hz, 1.8H); 0.84 (d, J=7.0 Hz, 1.2H); 0.86 (d, J=7.0 Hz, 1.8H); 0.88 (d, J=7.0 Hz, 1.2H); 1.00 (s, 3H); 1.04 (d, J=7.0 Hz, 3H); 1.11 (s, 3H); 1.30 (m, 4H); 1.57 (m, 2H); 1.80 (m, 1H); 1.95 (m, 1H); 2.26 (m, 1H); 2.40 (m, 2H); 2.52 to 2.72 (m, 6H); 2.95 to 3.04 (m, 3H); 3.22 (s, 3H); 3.30 (m, 1H); 3.38 to 3.63 (m, 18H); 3.80 (s, 3H); 3.87 (d, J=2.3 Hz, 1H); 4.15 to 4.28 (m, 4H); 4.40 (m, 1H); 4.51 to 4.57 (m, 2H); 4.90 (dd, J=3.8 and 9.8 Hz, 1H); 4.98 (s, 2H); 5.10 (m, 1H); 5.20 (m, 1H); 5.30 (m, 1H); 5.80 (d, J=16.0 Hz, 1H); 5.91 (m, 1H); 6.47 (ddd, J=4.7, 10.5 and 16.0 Hz, 1H); 7.05 (d, J=8.6 Hz, 1H); 7.16 (dd, J=2.5 and 8.6 Hz, 1H); 7.29 to 7.32 (m, 8H); 7.58 (m, 2H); 7.76 (t, J=6.5 Hz, 1H); 7.92 (d, J=9.0 Hz, 0.4H); 8.04 (d, J=9.0 Hz, 0.6H); 8.15 (d, J=7.0 Hz, 0.4H); 8.21 (d, J=7.0 Hz, 0.6H); 8.32 (d, J=8.0 Hz, 1H); 9.88 (s, 0.4H); 9.93 (s, 0.6H). LCMS (A): ES m/z=677; m/z=1418 [M+H]⁺; m/z=1462 [M−H+HCO₂H]⁻; t_{R}=1.21 min.

### Example 8: 15-(3-(((S)-1-(((S)-1-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)oxy)methyl)-phenyl)amino)-1-oxopropan-2-yl)amino)-3-methyl-1-oxobutan-2-yl)amino)-3-oxopropyl)-14-oxo-2,5,8,11-tetraoxa-15-azaoctadecan-18-oic acid

To a solution of compound 24 (58 mg, 40.9 μmol) in DCM (20 mL), were added, under Ar, 1,3-dimethylpyrimidine-2,4,6(1H, 3H, 5H)-trione (19.5 mg, 122.6 μmol) and tetrakis (triphenylphosphine)palladium (0) (2.4 mg, 2.0 μmol). The reaction medium was stirred for 3 h at RT, concentrated in vacuo and purified by flash chromatography on 5 g of silica gel (gradient elution DCM/MeOH) to give 53 mg of example 8 as a white solid (94%).

RMN ¹H (500 MHz, δ in ppm, DMSO-d6): conformer mixture; 0.78 (d, J=6.8 Hz, 6H); 0.86 (m, 6H); 0.99 (s, 3H); 1.04 (d, J=7.0 Hz, 3H); 1.11 (s, 3H); 1.30 (m, 1H); 1.38 (m, 3H); 1.57 (m, 2H); 1.79 (m, 1H); 2.04 (m, 1H); 2.10 to 2.59 (partially masked m, 7H); 2.69 (m, 2H); 2.93 to 3.03 (m, 3H); 3.22 (s, 3H); 3.30 (masked m, 1H); 3.40 to 3.64 (m, 18H); 3.80 (s, 3H); 3.87 (d, J=2.3 Hz, 1H); 4.05 to 4.27 (m, 4H); 4.36 (m, 1H); 4.90 (dd, J=3.8 and 9.8 Hz, 1H); 4.97 (s, 2H); 5.10 (m, 1H); 5.80 (d, J=16.0 Hz, 1H); 5.90 (ddd, J=4.7, 10.5 and 16.0 Hz, 1H); 6.46 (ddd, J=4.7, 10.5 and 16.0 Hz, 1H); 7.05 (d, J=8.6 Hz, 1H); 7.16 (dd, J=2.5 and 8.6 Hz, 1H); 7.20 to 7.32 (m, 8H); 7.68 (m, 2H); 7.82 (m, 1H); 8.40 to 8.65 (m, 2H); 9.21 (m, 1H); 10.20 (s, 1H). LCMS (A): ES m/z=637; m/z=1376 [M−H]⁻; m/z=1378 [M+H]⁺; t_{R}=1.3 min.

### Example 9: 2,5-dioxopyrrolidin-1-yl 15-(3-(((S)-1-(((S)-1-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)oxy)methyl)-phenyl)amino)-1-oxopropan-2-yl)amino)-3-methyl-1-oxobutan-2-yl)amino)-3-oxopropyl)-14-oxo-2,5,8,11-tetraoxa-15-azaoctadecan-18-oate

To a solution of example 8 (49 mg, 35.5 μmol) in DMF (2 mL), were added, under Ar, DSC (13.9 mg, 53.3 μmol) and DIEA (9.0 μL, 53.3 μmol). The reaction medium was stirred at RT overnight. Additional DSC (13.9 mg, 53.3 μmol) and DIEA (9.0 μL, 53.3 μmol) were added and the stirring carried over 8 h. The reaction medium was concentrated in vacuo and purified by flash chromatography on 10 g of diol-modified silica gel (gradient elution DCM/iPrOH) to give 20 mg of example 9 as a white solid (38%).

RMN ¹H (500 MHz, δ in ppm, DMSO-d6): 70:30 conformer mixture; 0.78 (d, J=6.8 Hz, 6H); 0.82 (d, J=7.0 Hz, 2.1H); 0.84 (d, J=7.0 Hz, 0.9H); 0.86 (d, J=7.0 Hz, 2.1H); 0.88 (d, J=7.0 Hz, 0.9H); 1.00 (s, 3H); 1.05 (d, J=7.0 Hz, 3H); 1.11 (s, 3H); 1.27 (m, 1H); 1.30 (m, 3H); 1.57 (m, 2H); 1.80 (m, 1H); 1.95 (m, 1H); 2.25 (m, 1H); 2.40 (m, 1H); 2.52 to 2.75 (m, 7H); 2.80 (s, 4H); 2.87 to 3.08 (m, 3H); 3.22 (s, 3H); 3.32 (masked m, 1H); 3.40 to 3.68 (m, 18H); 3.80 (s, 3H); 3.88 (d, J=2.3 Hz, 1H); 4.16 to 4.28 (m, 4H); 4.38 (m, 1H); 4.90 (dd, J=3.8 and 9.8 Hz, 1H); 4.97 (s, 2H); 5.10 (m, 1H); 5.79 (d, J=16.0 Hz, 1H); 6.46 (ddd, J=4.7, 10.5 and 16.0 Hz, 1H); 7.05 (d, J=8.6 Hz, 1H); 7.16 (dd, J=2.5 and 8.6 Hz, 1H); 7.20 to 7.31 (m, 8H); 7.58 (m, 2H); 7.78 (t, J=6.0 Hz, 1H); 7.94 (d, J=9.0 Hz, 0.3H); 8.08 (d, J=9.0 Hz, 0.7H); 8.19 (d, J=7.0 Hz, 0.3H); 8.25 (d, J=7.0 Hz, 0.7H); 8.35 (d, J=8.0 Hz, 1H); 9.90 (s, 0.3H); 9.95 (s, 0.7H). LCMS (A): ES m/z=698; m/z=1475 [M+H]⁺; t_{R}=1.35 min.

### Synthesis of Example 10: PEG7-Val-Ala-PABA-C52 benzylic amine

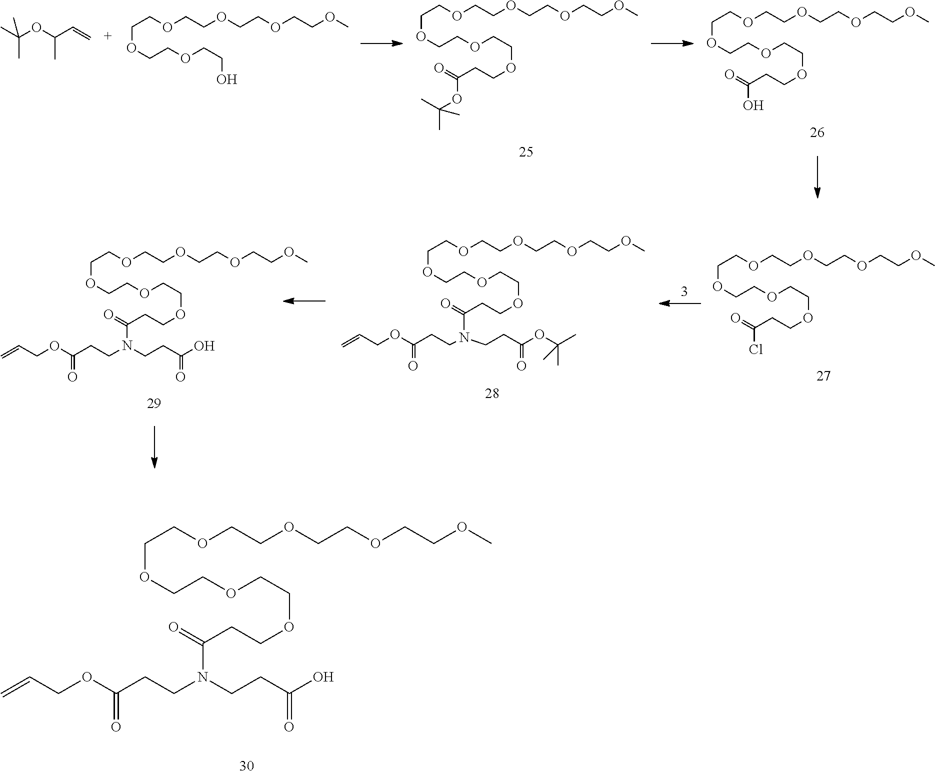

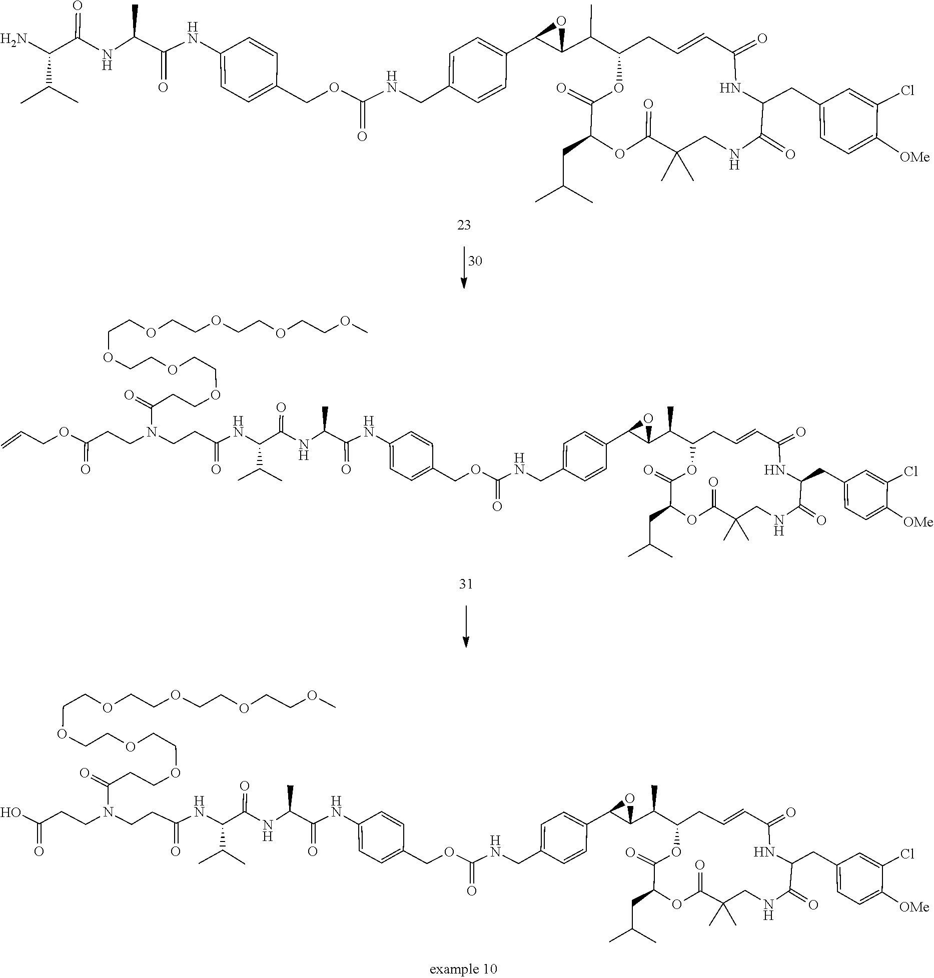

### Compound 25: tert-butyl 2,5,8,11,14,17,20-heptaoxatricosan-23-oate

To a solution of hexaethylene glycol monomethyl ether (926.0 μL, 3.24 mmol) in THF (3 mL), was added, under Ar, Na (0.7 mg, 32.4 μmol). The reaction medium was stirred for 1 h at RT until complete dissolution of Na then tert-butyl acrylate (570 μL, 3.89 mmol) was added and the stirring carried on at RT overnight. The reaction medium was then concentrated in vacuo, diluted with H₂O and extracted with EtOAc (3×). The combined organic phases were washed with brine, dried over MgSO₄, filtered and concentrated in vacuo to give 1.04 g of compound 25 as a colorless oil (76%).

RMN ¹H (400 MHz, δ in ppm, DMSO-d6): 1.40 (s, 9H); 2.40 (t, J=6.4 Hz, 2H); 3.23 (s, 3H); 3.43 (m, 2H); 3.47 to 3.54 (m, 22H); 3.59 (t, J=6.4 Hz, 2H).

### Compound 26: 2,5,8,11,14,17,20-heptaoxatricosan-23-oic acid

To a solution of compound 25 (1.04 g, 2.45 mmol) in DCM (15 mL) was added TFA (1.84 mL, 24.5 mmol). The reaction medium was stirred at RT overnight, concentrated in vacuo, diluted with DCM and co-evaporated with toluene (3×) to give 1 g of compound 26 as a colorless oil (quant.).

### Compound 27: 2,5,8,11,14,17,20-heptaoxatricosan-23-oyl chloride

To compound 26 (400 mg, 1.09 mmol) was added MsCl (1.27 mL, 16.3 mmol); the reaction medium was heated 3 h at 60° C., concentrated in vacuo, diluted with DCM and concentrated in vacuo (3×) to give 420 mg of compound 27 as a colorless oil (quant.).

### Compound 28: allyl 24-(3-(tert-butoxy)-3-oxopropyl)-23-oxo-2,5,8,11,14,17,20-heptaoxa-24-azaheptacosan-27-oate

To a solution of compound 3 (200 mg, 777.2 μmol) in DCM (5 mL) was added DIEA (262 μL, 1.55 mmol). The reaction medium was cooled down at 0° C. and a solution of compound 27 (360.8 mg, 932.7 μmol) in DCM (2 mL) was added dropwise at 0° C. The reaction medium was stirred for 1 h at RT and concentrated in vacuo. The crude product was purified by two consecutive flash chromatographies on 12 g and 10 g of silica gel (gradient elution DCM/MeOH) to give 72 mg of compound 28 (15%).

RMN ¹H (400 MHz, δ in ppm, DMSO-d6): 50:50 conformer mixture; 1.39 (s, 4.5H); 1.40 (s, 4.5H); 2.22 to 2.75 (partially masked m, 6H); 3.23 (s, 3H); 3.38 to 3.63 (m, 30H); 4.55 (m, 2H); 5.19 to 5.36 (m, 2H); 5.90 (m, 1H).

### Compound 29: 24-(3-(allyloxy)-3-oxopropyl)-23-oxo-2,5,8,11,14,17,20-heptaoxa-24-azahepta-cosan-27-oic acid

To a solution of compound 28 (72 mg, 118.5 μmol) in DCM (3 mL) was added TFA (100 μL, 1.33 mmol); the reaction medium was stirred at RT overnight then additional TFA (100 μL, 1.33 mmol) was added and the stirring carried on for 1 d. The reaction medium was concentrated in vacuo, diluted with DCM and co-evaporated with toluene (3×) to give 66 mg of compound 29 (quant.).

### Compound 30: allyl 24-(3-((2,5-dioxopyrrolidin-1-yl)oxy)-3-oxopropyl)-23-oxo-2,5,8,11,14,17,20-heptaoxa-24-azaheptacosan-27-oate

To a solution of compound 29 (66 mg, 119.7 μmol) in THF (2 mL), were added, under Ar, DIEA (20.2 μL, 119.9 μmol) and DSC (34.4 mg, 131.6 μmol); the reaction medium was stirred for 4 d at RT, concentrated in vacuo and purified by flash chromatography on 900 mg of diol-modified silica gel (gradient elution DCM/iPrOH) to give 35 mg of compound 30 (45%).

### Compound 31: allyl 24-(3-(((S)-1-(((S)-1-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclo-hexadec-13-en-16-yl)ethyl)oxiran-2-yl)benzyl)carbamoyl)oxy)-methyl)phenyl)amino)-1-oxopropan-2-yl)amino)-3-methyl-1-oxobutan-2-yl)amino)-3-oxopropyl)-23-oxo-2,5,8,11,14,17,20-heptaoxa-24-azaheptacosan-27-oate

To a solution of compound 23 (45 mg, 44.2 μmol) in THF (5 mL) were added compound 30 (28.7 mg, 44.2 μmol) and DIEA (11.2 μL, 66.3 μmol). The reaction medium was stirred at RT overnight, diluted with H₂O (10 mL) and extracted with EtOAc (3×10 mL). The combined organic phases were washed with brine, dried over MgSO₄, filtered, concentrated in vacuo and purified by flash chromatography on 5 g of silica gel (gradient elution DCM/MeOH) to give 35 mg of compound 31 as a white solid (51%).

RMN ¹H (500 MHz, δ in ppm, DMSO-d6): 60:40 conformer mixture; 0.80 (split d, J=6.8 Hz, 6H); 0.82 (d, J=7.0 Hz, 1.8H); 0.84 (d, J=7.0 Hz, 1.2H); 0.86 (d, J=7.0 Hz, 1.8H); 0.88 (d, J=7.0 Hz, 1.2H); 1.00 (s, 3H); 1.04 (d, J=7.0 Hz, 3H); 1.11 (s, 3H); 1.28 (m, 1H); 1.30 (m, 3H); 1.56 (m, 2H); 1.79 (m, 1H); 1.95 (m, 1H); 2.27 (m, 1H); 2.35 to 2.60 (partially masked m, 6H); 2.62 to 2.73 (m, 2H); 2.93 to 3.05 (m, 3H); 3.23 (s, 3H); 3.33 (masked m, 1H); 3.38 to 3.64 (m, 30H); 3.80 (s, 3H); 3.87 (d, J=2.3 Hz, 1H); 4.15 to 4.27 (m, 4H); 4.38 (m, 1H); 4.52 to 4.58 (m, 2H); 4.90 (dd, J=3.8 and 9.8 Hz, 1H); 4.97 (s, 2H); 5.10 (m, 1H); 5.50 (m, 1H); 5.30 (m, 1H); 5.80 (d, J=16.0 Hz, 1H); 5.90 (m, 1H); 6.47 (ddd, J=4.7, 10.5 and 16.0 Hz, 1H); 7.05 (d, J=8.6 Hz, 1H); 7.15 (dd, J=2.5 and 8.6 Hz, 1H); 7.20 to 7.32 (m, 8H); 7.59 (m, 2H); 7.78 (t, J=6.5 Hz, 1H); 7.95 (d, J=9.0 Hz, 0.4H); 8.07 (d, J=9.0 Hz, 0.6H); 8.18 (d, J=7.0 Hz, 0.4H); 8.27 (d, J=7.0 Hz, 0.6H); 8.36 (d, J=8.0 Hz, 1H); 9.90 (s, 0.4H); 9.95 (s, 0.6H). LCMS (A): ES m/z=698; m/z=1552 [M+H]⁺; m/z=1595 [M−H+HCO₂H]⁻; t_{R}=1.39 min.

### Example 10: 24-(3-(((S)-1-(((S)-1-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)oxy)methyl)-phenyl)amino)-1-oxopropan-2-yl)amino)-3-methyl-1-oxobutan-2-yl)amino)-3-oxopropyl)-23-oxo-2,5,8,11,14,17,20-heptaoxa-24-azaheptacosan-27-oic acid

To a solution of compound 31 (30 mg, 19.3 μmol) in DCM (5 mL) were added, under Ar, dimethylbarbituric acid (9.24 mg, 58.0 μmol) and tetrakis(triphenylphosphine)palladium (0) (1.13 mg, 0.97 μmol). The reaction medium was stirred for 3 h at RT then concentrated in vacuo and purified by two consecutive flash chromatographies on 5 g and 0.9 g of silica gel (gradient elution DCM/MeOH) to give 14 mg of example 10 as a white solid (50%).

RMN ¹H (500 MHz, δ in ppm, DMSO-d6): 50:50 conformer mixture; 0.78 (split d, J=6.8 Hz, 6H); 0.81 to 0.89 (m, 6H); 1.00 (s, 3H); 1.04 (d, J=7.0 Hz, 3H); 1.12 (s, 3H); 1.28 (m, 1H); 1.30 (d, J=7.0 Hz, 1.5H); 1.32 (d, J=7.0 Hz, 1.5H); 1.51 to 1.62 (m, 2H); 1.79 (m, 1H); 1.96 (m, 1H); 2.26 (m, 1H); 2.33 to 2.60 (partially masked m, 6H); 2.61 to 2.73 (m, 2H); 2.93 to 3.03 (m, 3H); 3.22 (s, 3H); 3.30 (masked m, 1H); 3.38 to 3.62 (m, 30H); 3.80 (s, 3H); 3.87 (d, J=2.3 Hz, 1H); 4.12 to 4.29 (m, 4H); 4.39 (m, 1H); 4.90 (dd, J=3.8 and 9.8 Hz, 1H); 4.96 (s, 2H); 5.10 (m, 1H); 5.78 (d, J=16.0 Hz, 1H); 6.46 (ddd, J=4.7, 10.5 and 16.0 Hz, 1H); 7.05 (d, J=8.6 Hz, 1H); 7.16 (dd, J=2.5 and 8.6 Hz, 1H); 7.20 to 7.32 (m, 8H); 7.59 (m, 2H); 7.79 (m, 1H); 8.04 (m, 0.5H); 8.10 (d, J=9.0 Hz, 0.5H); 8.25 to 8.41 (m, 2H); 9.95 (s, 0.5H); 9.98 (s, 0.5H); 12.20 (m, 1H). LCMS (D): ES m/z=769; m/z=1511 [M+H]⁺; t_{R}=3.16 min.

### Synthesis of Examples 11 & 12: PEG24-Val-Ala-PABA-C52 benzylic amine and NHS ester of PEG24-Val-Ala-PABA-C52 benzylic amine

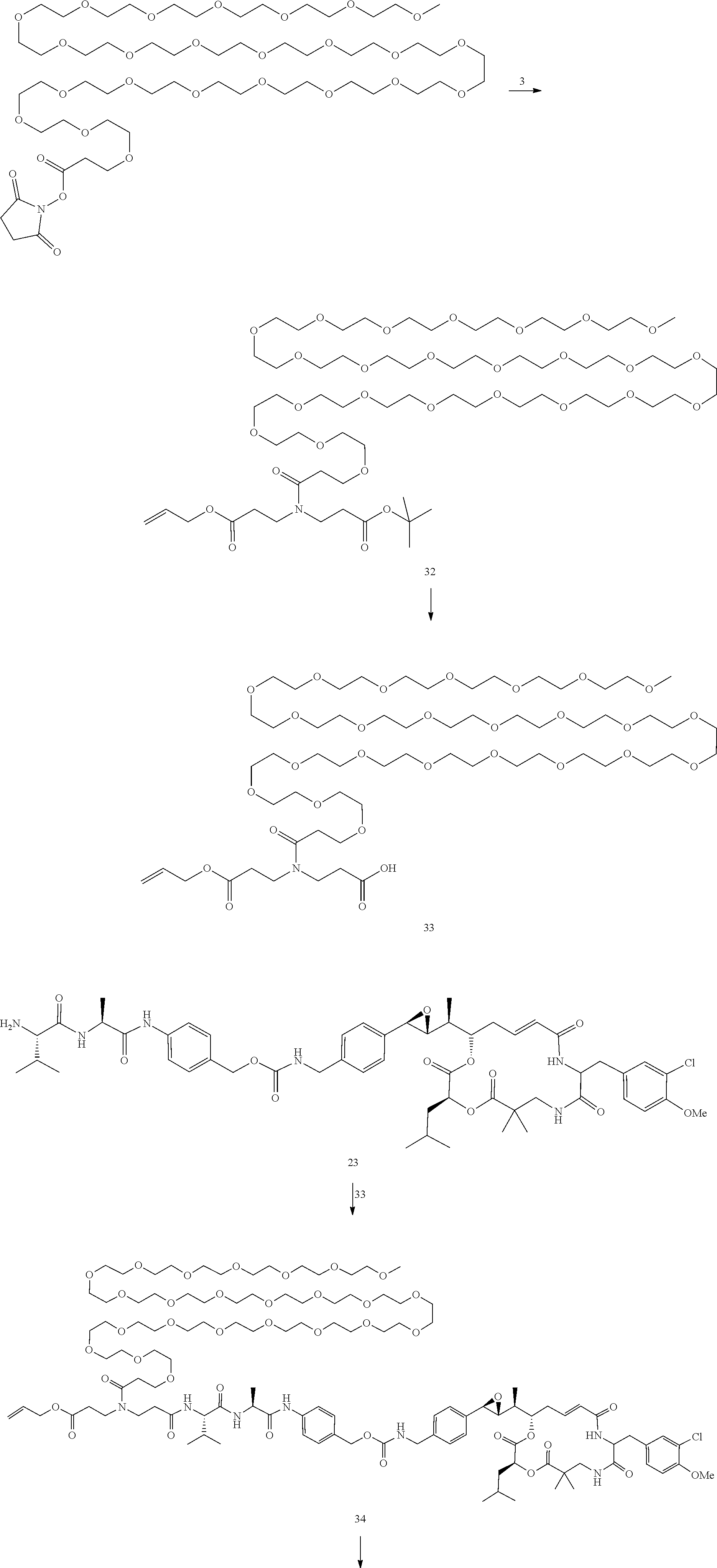

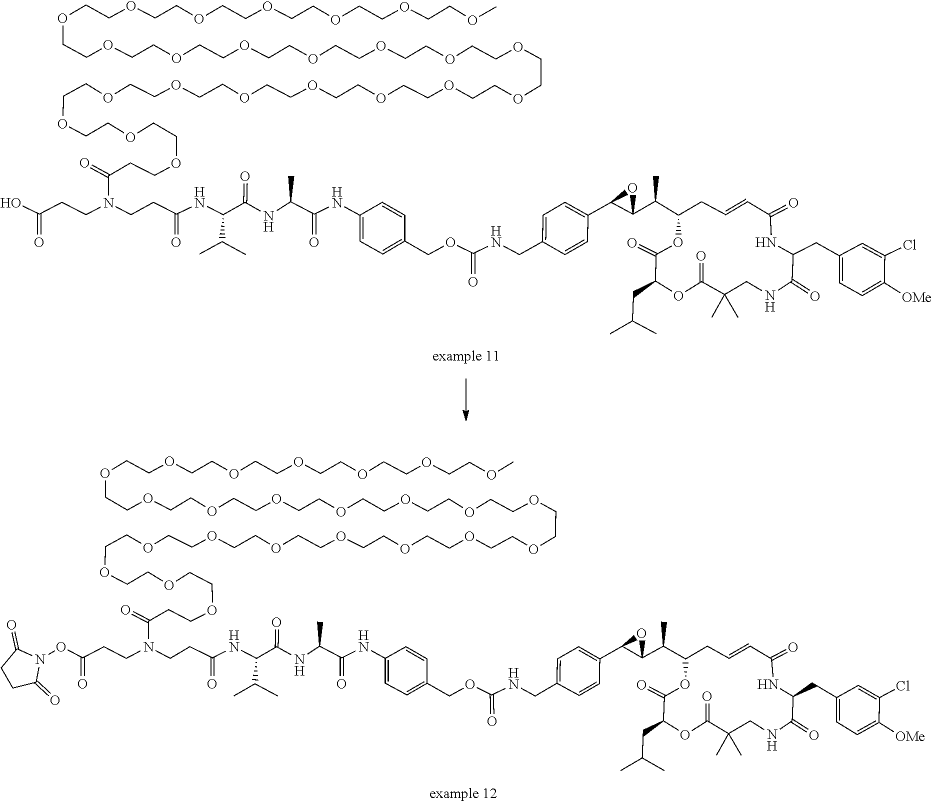

### Compound 32: allyl 75-(3-(tert-butoxy)-3-oxopropyl)-74-oxo-2,5,8,11,14,17,20,23,26,29,32,35,38,41,44,47,50,53,56,59,62,65,68,71-tetracosaoxa-75-azaoctaheptacontan-78-oate

To a solution of compound 3 (45 mg, 174.9 μmol) in THF (5 mL) were added, under Ar, MEO-DPEG(24)-NHS (212.4 mg, 174.9 μmol) and DIEA (44.2 μL, 262.3 μmol). The reaction medium was stirred at RT overnight then concentrated in vacuo and purified by flash chromatography on 20 g of silica gel (gradient elution DMC/MeOH) to give 125 mg of a mixture of compound 32 (60%) and MEO-DPEG(24)-NHS (40%). This mixture was diluted in THF (3 mL), 15 mg of compound 3 and 20 μL of DIEA were added; the reaction medium was stirred at RT overnight then concentrated in vacuo and purified by flash chromatography on 12 g of silica gel to give 105 mg of compound 32 (44%).

RMN ¹H (400 MHz, δ in ppm, DMSO-d6): 1.40 (s, 9H); 2.25 to 2.78 (m, 6H); 3.22 (s, 3H); 3.32 to 3.70 (m, 98H); 4.52 (m, 2H); 5.20 (m, 1H); 5.30 (m, 1H); 5.90 (m, 1H). LCMS (A): ES m/z=679 [M+2H]²⁺; m/z=1356 [M+H]⁺; m/z=1400 [M−H+HCO₂H]⁻; t_{R}=1.15 min.

### Compound 33: 75-(3-(allyloxy)-3-oxopropyl)-74-oxo-2,5,8,11,14,17,20,23,26,29,32, 35,38,41,44,47,50,53,56,59,62,65,68,71-tetracosaoxa-75-azaoctaheptacontan-78-oic acid

To a solution of compound 32 (105 mg, 77.4 μmol) in DCM (2 mL) was added TFA (116.2 μL, 1.55 mmol); the reaction medium was stirred at RT overnight, 150 μL of TFA were then added and the reaction carried on one more day. The reaction medium was then concentrated in vacuo, diluted with DCM and co-evaporated with toluene (3×) to give 110 mg of crude product that were purified by RP-HPLC on a 5 μm C18 SunFire column (Waters, 30×100 mm, gradient elution MeCN+0.07% TFA/H₂0+0.07% TFA) to provide 60 mg of compound 33 (60%).

RMN ¹H (400 MHz, δ in ppm, DMSO-d6): 50:50 conformer mixture; 2.37 to 2.60 (partially masked m, 6H); 3.23 (s, 3H); 3.32 to 3.44 (partially masked m, 38H); 3.47 to 3.52 (m, 58H); 3.60 (m, 2H); 4.54 (m, 2H); 5.22 (dm, J=10.5 Hz, 1H); 5.30 (dm, J=17.3 Hz, 1H); 5.84 to 5.98 (m, 1H); 12.32 (broad m, 1H).

### Compound 34: allyl 75-(3-(((S)-1-(((S)-1-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclo-hexadec-13-en-16-yl)ethyl)oxiran-2-yl)benzyl)carbamoyl)oxy)-methyl)phenyl)amino)-1-oxopropan-2-yl)amino)-3-methyl-1-oxobutan-2-yl)amino)-3-oxopropyl)-74-oxo-2,5,8,11,14,17,20,23,26,29,32,35,38,41,44,47,50,53,56,59,62,65,68,71-tetracosaoxa-75-azaoctaheptacontan-78-oate

To a solution of compound 33 (30 mg, 23.1 μmol) in THF (5 mL) were added, under Ar, DIEA (11.7 μL, 69.2 μmol) and DSC (6.03 mg, 23.1 μmol). The reaction medium was stirred for 3 h at RT then was added compound 23 (23.5 mg, 23.1 μmol) and the stirring carried on 1 h. The reaction medium was concentrated in vacuo and purified by flash chromatography on 5 g of silica gel to give 23 mg of compound 34 (40%).

RMN ¹H (500 MHz, δ in ppm, DMSO-d6): 60:40 conformer mixture: 0.80 (split d, J=6.8 Hz, 6H); 0.82 (d, J=7.0 Hz, 1.8H); 0.84 (d, J=7.0 Hz, 1.2H); 0.86 (d, J=7.0 Hz, 1.8H); 0.88 (d, J=7.0 Hz, 1.2H); 1.00 (s, 3H); 1.04 (d, J=7.0 Hz, 3H); 1.11 (s, 3H); 1.28 (m, 1H); 1.30 (d, J=7.0 Hz, 3H); 1.51 to 1.62 (m, 2H); 1.80 (m, 1H); 1.96 (m, 1H); 2.26 (m, 1H); 2.38 (m, 1H); 2.45 to 2.61 (partially masked m, 5H); 2.61 to 2.72 (m, 2H); 2.93 to 3.05 (m, 3H); 3.23 (s, 3H); 3.32 (masked m, 1H); 3.40 to 3.77 (m, 98H); 3.80 (s. 3H); 3.88 (d, J=2.3 Hz, 1H; 4.14 to 4.27 (m, 4H); 4.39 (m, 1H); 4.51 to 4.60 (m, 2H); 4.90 (dd, J=3.8 and 9.8 Hz, 1H); 4.97 (s, 2H); 5.10 (m, 1H); 5.18 to 5.33 (m, 2H); 5.78 (d, J=16.0 Hz, 1H); 5.90 (m, 1H); 6.45 (m, 1H); 7.05 (d, J=8.6 Hz, 1H); 7.16 (dd, J=2.5 and 8.6 Hz, 1H); 7.20 to 7.32 (m, 8H); 7.59 (m, 2H); 7.77 (t, J=6.5 Hz, 1H); 7.95 (d, J=9.0 Hz, 0.4H); 8.07 (d, J=9.0 Hz, 0.6H); 8.19 (d, J=7.0 Hz, 0.4H); 8.25 (d, J=7.0 Hz, 0.6H); 8.36 (d, J=8.0 Hz, 1H); 9.90 (s, 0.4H); 9.96 (s, 0.6H).

### Example 11: 75-(3-(((S)-1-(((S)-1-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)oxy)methyl)-phenyl)amino)-1-oxopropan-2-yl)amino)-3-methyl-1-oxobutan-2-yl)amino)-3-oxopropyl)-74-oxo-2,5,8,11,14,17,20,23,26,29,32,35,38,41,44,47,50,53,56,59,62,65, 68,71-tetracosaoxa-75-azaoctaheptacontan-78-oic acid

To a solution of compound 34 (37 mg, 161 μmol) in DCM were added, under Ar, 1,3-dimethylbarbituric acid (5.13 mg, 32.2 μmol) and tetrakis(triphenylphosphine)palladium(0) (0.94 mg, 0.80 μmol); the reaction medium was stirred at RT for 2 h then concentrated in vacuo and purified by flash chromatography on 5 g of diol-modified silica gel (gradient elution DCM/MeOH) to give 16 mg of example 11 (44%).

RMN ¹H (500 MHz, δ in ppm, DMSO-d6): 0.77 (d, J=7.0 Hz, 6H); 0.80 to 0.90 (m, 6H); 0.99 (s, 3H); 1.04 (d, J=7.0 Hz, 3H); 1.11 (s, 3H); 1.28 (m, 1H); 1.30 (d, J=7.0 Hz, 1.5H); 1.32 (d, J=7.0 Hz, 1.5H); 1.52 to 1.61 (m, 2H); 1.79 (m, 1H); 1.98 (m, 1H); 2.22 to 2.58 (partially masked m, 7H); 2.68 (m, 2H); 2.92 to 3.04 (m, 3H); 3.21 (s, 3H); 3.30 (partially masked m, 1H); 3.37 to 3.67 (m, 98H); 3.81 (s, 3H); 3.88 (s, 1H); 4.08 to 4.28 (m, 4H); 4.39 (m, 1H); 4.90 (m, 1H); 4.98 (s, 2H); 5.10 (m, 1H); 5.80 (d, J=16.0 Hz, 1H); 6.48 (ddd, J=4.7, 10.5 and 16.0 Hz, 1H); 7.04 (d, J=8.6 Hz, 1H); 7.16 (broad d, J=8.6 Hz, 1H); 7.20 to 7.32 (m, 8H); 7.57 to 7.67 (m, 2H); 7.80 (m, 1H); 8.20 (m, 1H); 8.35 to 8.70 (m, 2H); 10.08 (m, 1H); 12.30 (broad m, 1H). LCMS (D): ES m/z=698; m/z=2259 [M+H]⁺; t_{R}=3.14 min.

### Example 12: 2,5-dioxopyrrolidin-1-yl 75-(3-(((S)-1-(((S)-1-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)-carbamoyl)oxy)methyl)phenyl)amino)-1-oxopropan-2-yl)amino)-3-methyl-1-oxobutan-2-yl)amino)-3-oxopropyl)-74-oxo-2,5,8,11,14,17,20,23,26,29,32,35,38,41,44,47,50, 53,56,59,62,65,68,71-tetracosaoxa-75-azaoctaheptacontan-78-oate

To a solution of example 11 (16 mg, 7.1 μmol) in DCM were added DIEA (1.2 μL, 7.1 μmol) and DSC (1.8 mg, 7.1 μmol). The reaction medium was stirred for 3 h at RT then concentrated in vacuo and purified by flash chromatography on 1.3 g of diol-modified silica gel (gradient elution DCM/iPrOH) to give 5 mg of a mixture of example 12 (30%) and example 11 (70%).

LCMS (D): ES m/z=698; m/z=2356 [M+H]⁺; t_{R}=3.2 min.

### Synthesis of Example 13: sulfo-PEG4-Val-Ala-PABA-C52 benzylic amine

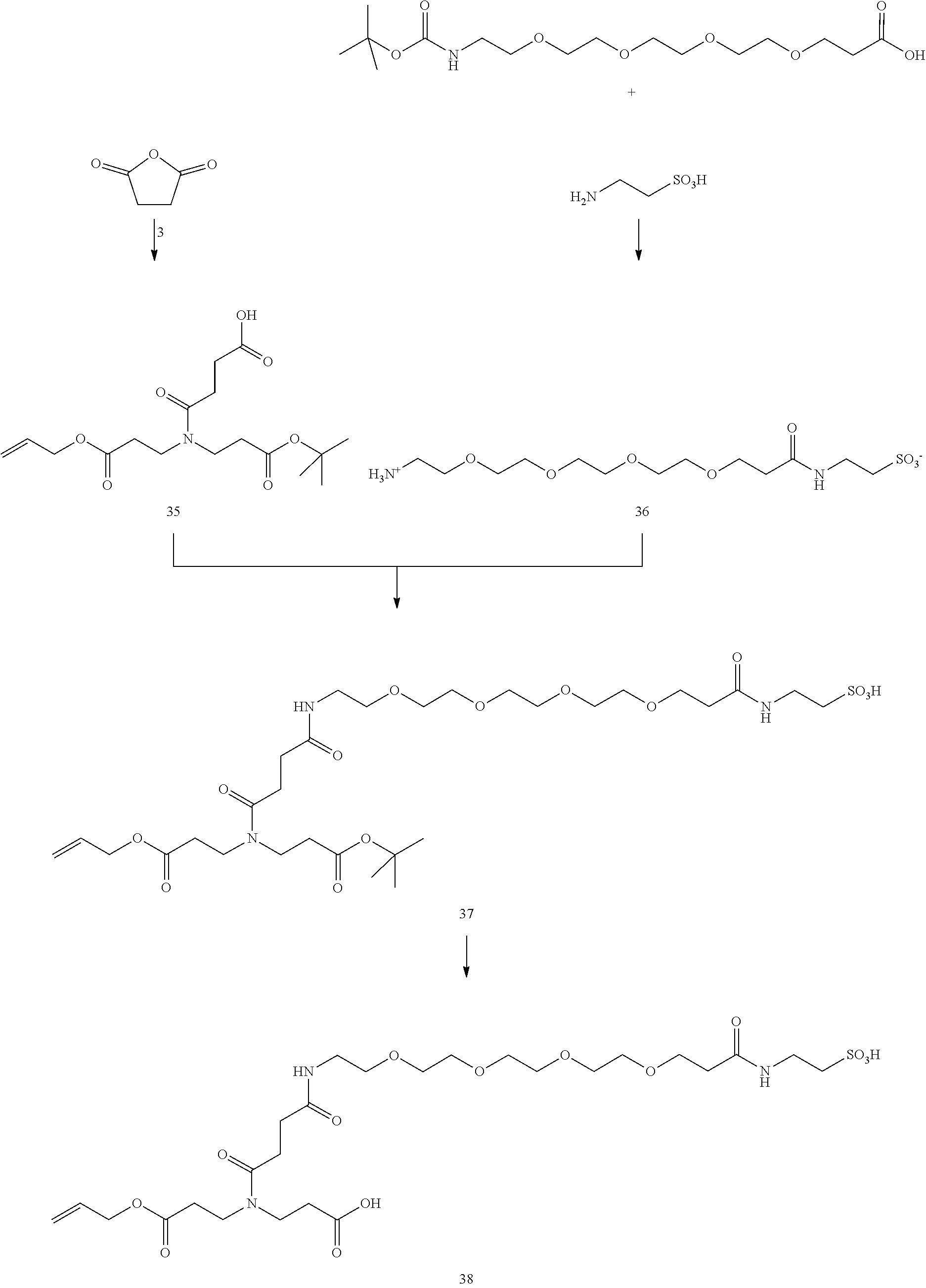

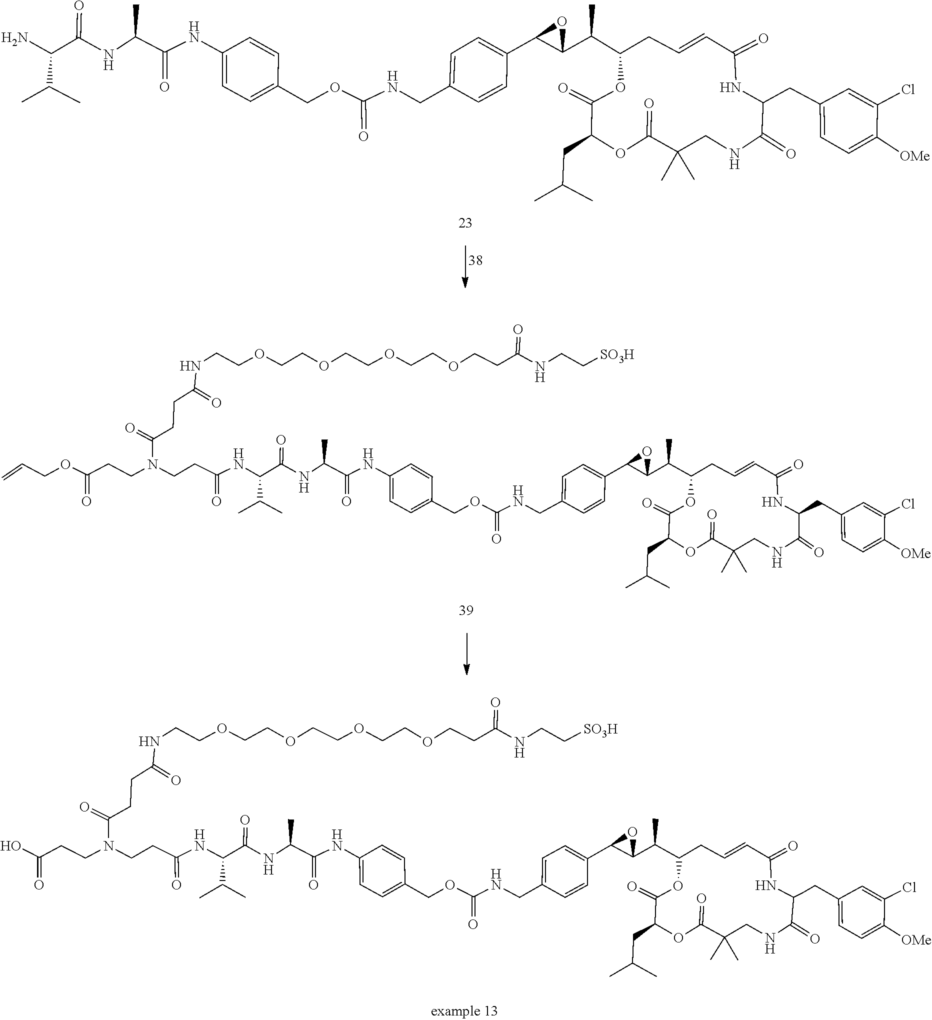

### Compound 35: 4-((3-(allyloxy)-3-oxopropyl)(3-(tert-butoxy)-3-oxopropyl)amino)-4-oxobutanoic acid

To a solution of compound 3 (100 mg, 388.6 μmol) in DCM (5 mL) was added succinic anhydride (78.6 mg, 777.2 μmol). The reaction medium was stirred at RT overnight then concentrated in vacuo and purified by flash chromatography on 4 g of silica gel (gradient elution DCM/MeOH) to give 102 mg of compound 35 as a colorless oil (73%).

### Compound 36: 1-ammonio-15-oxo-3,6,9,12-tetraoxa-16-azaoctadecane-18-sulfonate

To a solution of 15-(Boc-amino)-4,7,10,13-tetraoxapentadecanoic acid (500 mg, 1.3 mmol) in THF (5 mL) were added, under Ar, DIEA (438.4 μL, 2.6 mmol) and DSC (381.6 mg, 1.43 mmol). The reaction medium was stirred for 6 h at RT then 50 mg of DSC were added and the stirring was carried on at RT overnight. At that time, taurine (821.6 mg, 6.5 mmol) and H₂O (1 mL) were added to the reaction medium. The reaction medium was stirred at RT overnight, concentrated in vacuo and purified by preparative LCMS on a 5 μm C18 SunFire column (Waters, 30×100 mm, gradient elution MeCN+0.07% TFA/H₂O+0.07% TFA) to give 341 mg of compound 36 (70%).

RMN ¹H (400 MHz, δ in ppm, DMSO-d6): 2.28 (t, J=6.5 Hz, 2H); 2.60 (m, 2H); 2.99 (m, 2H); 3.32 (m, 2H); 3.45 to 3.65 (m, 16H); 7.77 (broad m, 4H). IR spectrum as a KBr pellet; main absorption bands in reciprocal centimeters: 3000; 1780; 1648; 1556; 1200-1170; 1200; 1100; 1040.

### Compound 37: 24-(3-(tert-butoxy)-3-oxopropyl)-4,20,23,27-tetraoxo-7,10,13,16,28-pentaoxa-3,19,24-triazahentriacont-30-ene-1-sulfonate

To a solution of compound 35 (100 mg, 279.8 μmol) in THF were added DIEA (141.6 μL, 839.4 μmol) and DSC (73.1 mg, 279.8 μmol). The reaction mixture was stirred for 2 h at RT then a solution of compound 36 (104.2 mg, 279.8 μmol) in DMF and DIEA (141.6 μL, 839.4 μmol) were added. The reaction medium was stirred for 2 h at RT, concentrated in vacuo and purified by preparative LCMS on a 5 μm C18 SunFire column (Waters, 30×100 mm, gradient elution MeCN+0.07% TFA/H₂O+0.07% TFA) to give 118 mg of compound 37 (60%).

RMN ¹H (400 MHz, δ in ppm, DMSO-d6): 50:50 conformer mixture; 1.39 (s, 4.5H); 1.40 (s, 4.5H); 2.27 (t, J=6.5 Hz, 2H); 2.31 (t, J=7.0 Hz, 2H); 2.38 (t, J=7.0 Hz, 1H); 2.45 to 2.56 (masked m, 6H); 2.68 (m, 1H); 3.18 (q, J=6.0 Hz, 2H); 3.29 (m, 2H); 3.36 to 3.60 (m, 20H); 4.51 to 4.59 (m, 2H); 5.17 to 5.25 (m, 2H); 5.85 to 5.95 (m, 1H); 7.75 (t, J=6.0 Hz, 1H); 7.88 (t, J=6.0 Hz, 1H).

### Compound 38: 24-(2-carboxyethyl)-4,20,23,27-tetraoxo-7,10,13,16,28-pentaoxa-3,19,24-triazahentriacont-30-ene-1-sulfonate

To a solution of compound 37 (118 mg, 166.0 μmol) in DCM (5 mL) was added TFA (249 μL, 3.32 mmol). The reaction medium was stirred at RT overnight, concentrated in vacuo, diluted with DCM and co-evaporated with toluene (3×) to give 100 mg of compound 38 as a colorless oil (92%).

### Compound 39: (2S,5S)-10-(3-(allyloxy)-3-oxopropyl)-1-((4-((((4-((2R,3R)-3-((S)-1-((3S,10R, 16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)oxy)methyl)phenyl)amino)-5-isopropyl-2-methyl-1,4,7,11,14,30-hexaoxo-18,21,24,27-tetraoxa-3,6,10,15,31-pentaazatritria-contane-33-sulfonic acid

To a solution of compound 38 (100 mg, 152.7 μmol) in THF (3 mL) and DMF (1 mL) were added DIEA (77 μL, 458.2 μmol) and DSC (39.9 mg, 152.7 μmol). The reaction medium was stirred for 2 h at RT before the addition of a solution of compound 23 (124 mg, 121.9 μmol) in THF (1 mL) and DMF (0.5 mL) and DIEA (100 μL). The reaction medium was stirred for 2 h at RT, concentrated in vacuo and purified by reverse phase flash chromatography on 75 g of C18-modified silica gel (gradient elution H₂O/MeCN) to give 60 mg of compound 39 as a white solid (24%).

RMN ¹H (500 MHz, δ in ppm, DMSO-d6): 60:40 conformer mixture; 0.78 (d, J=6.8 Hz, 6H); 0.82 (d, J=7.0 Hz, 1.8H); 0.84 (d, J=7.0 Hz, 1.2H); 0.86 (d, J=7.0 Hz, 1.8H); 0.88 (d, J=7.0 Hz, 1.2H); 0.99 (s, 3H); 1.04 (d, J=7.0 Hz, 3H); 1.11 (s, 3H); 1.28 (m, 1H); 1.30 (d, J=7.0 Hz, 3H); 1.50 to 1.62 (m, 2H); 1.80 (m, 1H); 1.96 (m, 1H); 2.20 to 2.55 (partially masked m, 11H); 2.61 to 2.75 (m, 2H); 2.94 to 3.05 (m, 3H); 3.10 to 3.65 (partially masked, 27H); 3.80 (s, 3H); 3.87 (d, J=2.3 Hz, 1H); 4.13 to 4.28 (m, 4H); 4.38 (m, 1H); 4.53 (m, 1.2H); 4.56 (m, 0.8H); 4.90 (dd, J=3.8 and 9.8 Hz, 1H); 4.97 (s, 2H); 5.10 (m, 1H); 5.18 to 5.33 (m, 2H); 5.80 (d, J=16.0 Hz, 1H); 5.90 (m, 1H); 6.47 (ddd, J=4.7, 10.5 and 16.0 Hz, 1H); 7.05 (d, J=8.6 Hz, 1H); 7.16 (dd, J=2.5 and 8.6 Hz, 1H); 7.20 to 7.32 (m, 8H); 7.59 (m, 2H); 7.72 (t, J=6.5 Hz, 1H); 7.79 (t, J=6.0 Hz, 1H); 7.89 (t, J=6.5 Hz, 1H); 7.95 (d, J=9.0 Hz, 0.4H); 8.08 (d, J=9.0 Hz, 0.6H); 8.19 (d, J=7.0 Hz, 0.4H); 8.25 (d, J=7.0 Hz, 0.6H); 8.36 (d, J=8.0 Hz, 1H); 9.90 (s, 0.4H); 9.95 (s, 0.6H). LCMS (D): ES m/z=913; m/z=1654 [M+H]⁺; t_{R}=3.87 min.

### Example 13: 24-(3-(((S)-1-(((S)-1-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)oxy)methyl)-phenyl)amino)-1-oxopropan-2-yl)amino)-3-methyl-1-oxobutan-2-yl)amino)-3-oxopropyl)-4,20,23-trioxo-1-sulfo-7,10,13,16-tetraoxa-3,19,24-triazaheptacosan-27-oic acid

To a solution of compound 39 (60 mg, 36.3 μmol) in DMF (5 mL) were added 1,3-dimethylbarbituric acid (17.3 mg, 108.8 μmol) and tetrakis (triphenylphosphine)palladium(0) (2.1 mg, 1.8 μmol). The reaction medium was stirred at RT overnight, concentrated in vacuo and purified by two flash chromatographies on 30 g and 20 g of C18-modified silica gel to give two batches of 6 mg of example 13 as a white solid (20% global yield).

RMN ¹H (500 MHz, δ in ppm, DMSO-d6): 60:40 conformer mixture; 0.78 (d, J=6.8 Hz, 6H); 0.81 to 0.89 (m, 6H); 1.00 (s, 3H); 1.04 (d, J=7.0 Hz, 3H); 1.11 (s, 3H); 1.28 (m, 1H); 1.30 (d, J=7.0 Hz, 3H); 1.58 (m, 2H); 1.80 (m, 1H); 1.96 (m, 1H); 2.21 to 2.58 (partially masked m, 11H); 2.69 (m, 2H); 2.93 to 3.68 (partially masked m, 28H); 3.80 (s, 3H); 3.88 (d, J=2.3 Hz, 1H); 4.11 to 4.28 (m, 4H); 4.38 (m, 1H); 4.90 (dd, J=3.8 and 9.8 Hz, 1H); 4.98 (s, 2H); 5.10 (m, 1H); 5.79 (d, J=16.0 Hz, 1H); 6.47 (ddd, J=4.7, 10.5 and 16.0 Hz, 1H); 7.05 (d, J=8.6 Hz, 1H); 7.17 (dd, J=2.5 and 8.6 Hz, 1H); 7.20 to 7.32 (m, 8H); 7.59 (m, 2H); 7.72 (t, J=6.5 Hz, 1H); 7.79 (t, J=6.0 Hz, 1H); 7.89 (t, J=6.5 Hz, 1H); 7.97 (d, J=9.0 Hz, 0.4H); 8.08 (d, J=9.0 Hz, 0.6H); 8.19 (d, J=7.0 Hz, 0.4H); 8.25 (d, J=7.0 Hz, 0.6H); 8.36 (d, J=8.0 Hz, 1H); 9.90 (s, 0.4H); 9.94 (s, 0.6H); 12.15 (broad m, 1H). LCMS (A): ES m/z=807.5 [M+2H]²⁺; m/z=1612 [M−H]⁻; m/z=1614 [M+H]⁺; t_{R}=1.72 min.

### Synthesis of Examples 14 to 16: glutaryl-Val-PEG4Gln-C52 benzylic amine, NHS ester of glutaryl-Val-PEG4Gln-C52 benzylic amine and Corresponding ADC

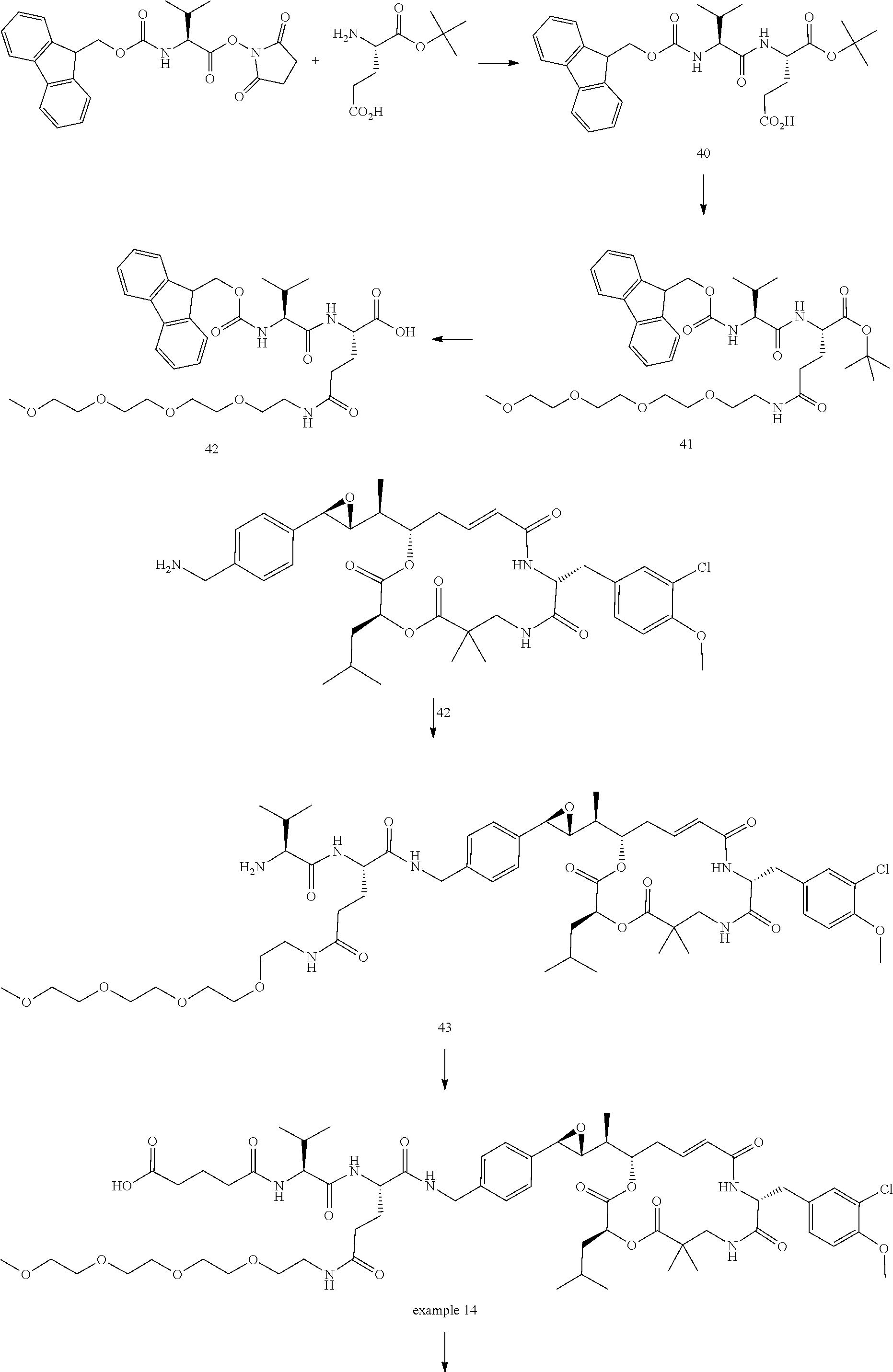

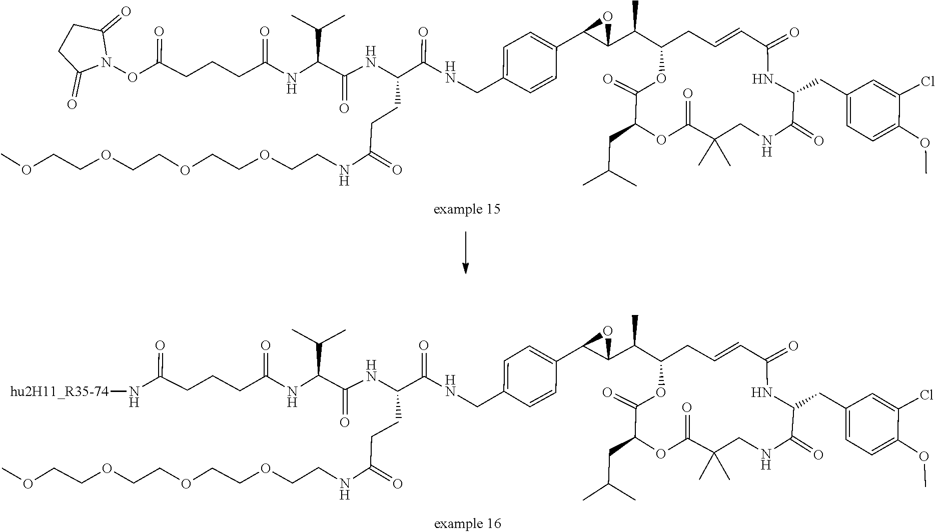

### Compound 40: (S)-4-((S)-2-((((9H-fluoren-9-yl)methoxy)carbonyl)amino)-3-methylbutanamido)-5-(tert-butoxy)-5-oxopentanoic acid

To a solution of H-Glu-OtBu (CAS number [45120-30-7], 959 mg, 4.7 mmol) and NaHCO₃ (400 mg, 4.77 mmol) in H₂O (80 mL), was added dropwise, under magnetic stirring, a solution of (S)-2,5-dioxopyrrolidin-1-yl 2-((((9H-fluoren-9-yl)methoxy)-carbonyl)amino)-3-methylbutanoate (2 g, 4.6 mmol) in THF (80 mL). The reaction medium was stirred at RT overnight. The medium was concentrated in vacuo, then saturated aqueous NaHCO₃ (1 L) was added, followed by extraction with Et₂O (2×250 mL), organic phases were excluded. The aqueous phase was acidified with QS of aqueous 1 N HCl to reach pH 2, and extracted with DCM (3×250 mL), the combined organic phases were dried over MgSO₄, filtered and concentrated in vacuo to give 2.05 g compound 40 (85%).

RMN ¹H (400 MHz, δ in ppm, DMSO-d6): 0.87 (d, J=6.9 Hz, 3H); 0.90 (d, J=6.9 Hz, 3H); 1.38 (s, 9H); 1.73 to 2.02 (m, 3H); 2.24 to 2.30 (m, 2H); 3.90 (dd, J=7.3 and 9.0 Hz, 1H); 4.14 (m, 1H); 4.19 to 4.32 (m, 3H); 7.27 to 7.45 (m, 5H); 7.75 (t, J=7.6 Hz, 2H); 7.89 (d, J=7.6 Hz, 2H); 8.22 (broad d, J=7.0 Hz, 1H); 12.05 (broad s, 1H).

### Compound 41: (S)-tert-butyl 18-((S)-2-((((9H-fluoren-9-yl)methoxy)carbonyl)amino)-3-methylbutanamido)-15-oxo-2,5,8,11-tetraoxa-14-azanonadecan-19-oate

To a solution of compound 40 (200 mg, 381 μmol) in DCM (6 mL) were added, under magnetic stirring, PEG4-NH₂ (CAS number [85030-56-4], 115 mg, 554.9 μmol), HOBt (82 mg, 606.8 μmol) and EDC (100 μL, 565 μmol). The reaction medium was stirred at RT overnight. The crude medium was purified twice by flash chromatography on silica gel (gradient elution DCM/MeOH) to give 190 mg of compound 41 (70%).

RMN ¹H (400 MHz, δ in ppm, DMSO-d6): 0.87 (d, J=6.8 Hz, 3H); 0.90 (d, J=6.8 Hz, 3H); 1.38 (s, 9H); 1.72 to 1.93 (m, 2H); 1.99 (m, 1H); 2.14 (t, J=7.8 Hz, 2H); 3.18 (m, 2H); 3.23 (s, 3H); 3.37 (t, J=6.0 Hz, 2H); 3.41 (m, 2H); 3.46 to 3.53 (m, 10H); 3.92 (dd, J=7.1 and 8.8 Hz, 1H); 4.09 (m, 1H); 4.17 to 4.34 (m, 3H); 7.32 (m, 3H); 7.41 (t, J=7.6 Hz, 2H); 7.70 to 7.81 (m, 3H); 7.88 (d, J=7.6 Hz, 2H); 8.18 (d, J=7.3 Hz, 1H). LCMS (A): ES m/z=714 [M+H]⁺; m/z=758 [M−H+HCO₂H]⁻; t_{R}=1.41 min.

### Compound 42: (S)-18-((S)-2-((((9H-fluoren-9-yl)methoxy)carbonyl)amino)-3-methylbutanamido)-15-oxo-2,5,8,11-tetraoxa-14-azanonadecan-19-oic acid

To compound 41 (190 mg, 266 μmol) was added, under magnetic stirring, a 4M solution of HCl in 1,4-dioxane (5 mL). The reaction medium was stirred at RT for 4 h. At this time, the reaction medium was concentrated in vacuo and purified by flash chromatography on 15 g of silica gel (gradient elution DCM/MeOH/NH₄OH) to give 70 mg of compound 42 (40%).

RMN ¹H (400 MHz, δ in ppm, DMSO-d6): 0.84 (d, J=6.6 Hz, 3H); 0.86 (d, J=6.6 Hz, 3H); 1.70 to 1.90 (m, 2H); 2.01 to 2.15 (m, 3H); 3.15 (m, 2H); 3.23 (s, 3H); 3.36 (t, J=6.4 Hz, 2H); 3.42 (m, 2H); 3.45 to 3.53 (m, 10H); 3.80 (broad m, 1H); 3.83 (dd, J=6.8 and 8.7 Hz, 1H); 4.19 to 4.34 (m, 3H); 7.33 (m, 2H); 7.41 (t, J=7.6 Hz, 2H); 7.59 (broad m, 2H); 7.73 (d, J=7.6 Hz, 1H); 7.76 (d, J=7.6 Hz, 1H); 7.89 (d, J=7.6 Hz, 2H); 8.00 (broad m, 1H). LCMS (A): ES m/z=656 [M−H]⁻; m/z=658 [M+H]⁺; RT=1.19 min.

### Compound 43: (S)-2-((S)-2-amino-3-methylbutanamido)-N1-(4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)-N5-(2,5,8,11-tetraoxatridecan-13-yl)pentanediamide

To a solution of (E)-(3S,10R,16S)-16-{(S)-1-[(2R,3R)-3-(4-aminomethyl-phenyl)-oxiranyl]-ethyl}-10-(3-chloro-4-methoxy-benzyl)-3-isobutyl-6,6-dimethyl-1,4-dioxa-8,11-diaza-cyclohexadec-13-ene-2,5,9,12-tetraone (the synthesis of which was described in WO2011001052, compound 77, 58 mg, 83 μmol) in DMF (5 mL), under magnetic stirring, were added compound 42 (70 mg, 106 μmol), HOBt (20 mg, 148 μmol) and EDC (23 μL, 130 μmol). The reaction medium was stirred at RT for 24 h. At this time, piperidine (80 μL, 810 μmol) was added to the medium, and stirring was maintained for 1 h. Then, the reaction medium was concentrated in vacuo and purified by flash chromatography on 20 g silica gel (gradient elution DCM/MeOH) to give 60 mg of compound 43 (65%).

RMN ¹H (500 MHz, δ in ppm, DMSO-d6): 0.77 (d, J=6.9 Hz, 3H); 0.79 (d, J=6.3 Hz, 6H); 0.87 (d, J=6.9 Hz, 3H); 1.00 (s, 3H); 1.04 (d, J=6.4 Hz, 3H); 1.12 (s, 3H); 1.31 (m, 1H); 1.52 to 1.63 (m, 2H); 1.72 to 1.98 (m, 4H); 2.05 to 2.15 (m, 2H); 2.26 (m, 1H); 2.62 to 2.73 (m, 2H); 2.94 to 3.04 (m, 4H); 3.18 (q, J=5.9 Hz, 2H); 3.23 (s, 3H); 3.33 (partially masked m, 1H); 3.39 (t, J=5.9 Hz, 2H); 3.42 (m, 2H); 3.47 to 3.52 (m, 10H); 3.81 (s, 3H); 3.87 (d, J=1.4 Hz, 1H); 4.21 to 4.36 (m, 4H); 4.91 (dd, J=3.6 and 9.6 Hz, 1H); 5.10 (dd, J=5.5 and 11.8 Hz, 1H); 5.79 (d, J=15.3 Hz, 1H); 6.46 (ddd, J=3.8, 11.6 and 15.3 Hz, 1H); 7.05 (d, J=8.5 Hz, 1H); 7.17 (dd, J=1.9 and 8.5 Hz, 1H); 7.21 to 7.25 (m, 5H); 7.28 (d, J=1.9 Hz, 1H); 7.89 (t, J=5.5 Hz, 1H); 8.07 (m large, 1H); 8.37 (d, J=8.0 Hz, 1H); 8.45 (t, J=5.9 Hz, 1H). LCMS (A): ES m/z=558 [M+2H]²⁺; m/z=1115 [M+H]⁺; m/z=1159 [M−H+HCO₂H]⁻; t_{R}=0.96 min.

### Example 14: (18S,21S)-18-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)-21-isopropyl-15,20,23-trioxo-2,5,8,11-tetraoxa-14,19,22-triazaheptacosan-27-oic acid

To a solution of compound 43 (55 mg, 49.3 μmol) in DMF (3 mL) was added glutaric anhydride (9 mg, 79 μmol), the reaction medium was stirred at RT for 2 h. At this time, the medium was concentrated in vacuo and purified by flash chromatography on 10 g of silica gel (gradient elution DCM/MeOH) to give 38 mg of example 14 (62%).

RMN ¹H (500 MHz, δ in ppm, DMSO-d6): 0.81 (split d, J=6.6 Hz, 6H); 0.85 (d, J=6.3 Hz, 3H); 0.87 (d, J=6.3 Hz, 3H); 0.98 (s, 3H); 1.03 (d, J=6.6 Hz, 3H); 1.11 (s, 3H); 1.35 (m, 1H); 1.52 to 2.34 (m, 15H); 2.71 (m, 2H); 2.89 to 3.02 (m, 3H); 3.09 to 3.21 (m, 2H); 3.23 (s, 3H); 3.30 to 3.40 (partially masked m, 3H); 3.42 (m, 2H); 3.46 to 3.51 (m, 10H); 3.81 (s, 3H); 3.87 (s, 1H); 4.03 to 4.24 (m, 4H); 4.37 (m, 1H); 4.96 (m, 1H); 5.11 (m, 1H); 5.81 (d, J=15.3 Hz, 1H); 6.46 (ddd, J=3.3, 11.4 and 15.3 Hz, 1H); 7.04 (d, J=8.5 Hz, 1H); 7.19 (dd, J=1.9 and 8.5 Hz, 1H); 7.22 (d, J=8.3 Hz, 2H); 7.27 (d, J=8.3 Hz, 2H); 7.30 (d, J=1.9 Hz, 1H); 7.32 (m, 1H); 7.85 to 8.99 (broad m, 5H); 12.12 (broad m, 1H). LCMS (A): ES m/z=615 [M+2H]²⁺; m/z=1227 [M−H]⁻; m/z=1229 [M+H]⁺; t_{R}=1.26 min.

### Example 15: (18S,21S)-2,5-dioxopyrrolidin-1-yl 18-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)-21-isopropyl-15,20,23-trioxo-2,5,8,11-tetraoxa-14,19,22-triazaheptacosan-27-oate

To a solution of example 14 (32 mg, 26 μmol) in DMF (3 mL), under magnetic stirring, were added DSC (8 mg, 31.2 μmol) and DIEA (8.6 μL, 52 μmol). The reaction medium was stirred at RT overnight. Then, 4 mg of DSC were added and stirring at RT was maintained for 1 h. At this time, the reaction medium was diluted with MeTHF (10 mL), washed with H₂O (5 mL). The aqueous phase was extracted with MeTHF (3×5 mL). The combined organic phases were dried over MgSO₄, filtered and concentrated in vacuo. The medium was purified by flash chromatography on 4 g of silica gel (gradient elution DCM/iPrOH) to give 28 mg of example 15 (81%).

RMN ¹H (500 MHz, δ in ppm, DMSO-d6): 0.78 (d, J=6.6 Hz, 6H); 0.83 (d, J=6.9 Hz, 3H); 0.85 (d, J=6.9 Hz, 3H); 1.00 (s, 3H); 1.04 (d, J=6.4 Hz, 3H); 1.12 (s, 3H); 1.30 (m, 1H); 1.52 to 1.64 (m, 2H); 1.75 to 1.93 (m, 5H); 1.98 (m, 1H); 2.12 (m, 2H); 2.22 to 2.34 (m, 3H); 2.62 to 2.72 (m, 4H); 2.81 (s, 4H); 2.93 to 3.04 (m, 3H); 3.18 (q, J=6.0 Hz, 2H); 3.23 (s, 3H); 3.33 (partially masked m, 1H); 3.39 (t, J=6.0 Hz, 2H); 3.42 (m, 2H); 3.47 to 3.52 (m, 10H); 3.81 (s, 3H); 3.87 (d, J=1.6 Hz, 1H); 4.15 (dd, J=6.6 and 8.2 Hz, 1H); 4.19 to 4.32 (m, 4H); 4.91 (dd, J=3.6 and 9.6 Hz, 1H); 5.10 (m, 1H); 5.79 (d, J=15.3 Hz, 1H); 6.47 (ddd, J=3.7, 11.3 and 15.3 Hz, 1H); 7.05 (d, J=8.5 Hz, 1H); 7.17 (dd, J=2.2 and 8.5 Hz, 1H); 7.21 to 7.26 (m, 5H); 7.28 (d, J=2.2 Hz, 1H); 7.85 (t, J=6.0 Hz, 1H); 7.92 (d, J=8.2 Hz, 1H); 8.08 (d, J=7.7 Hz, 1H); 8.32 (t, J=6.0 Hz, 1H); 8.37 (d, J=8.0 Hz, 1H). LCMS (A): ES m/z=1326 [M+H]⁺; m/z=1348 [M+Na]⁺; m/z=1370 [M−H+HCO₂H]⁻; t_{R}=1.31 min.

### Example 16: hu2H11_R35-74-Ex15

The general method described previously was used for the preparation of example 16. 28.8 mg of hu2H11_R35-74 were reacted with 115.2 μL of a 10.05 mM solution of example 15 in DMA (6 eq.) for 2 h 30. After purification on Superdex 200 μg in buffer B pH 6.5+10% NMP, concentration on Amicon Ultra-15, dilution in buffer B pH 6.5 to a final concentration of NMP at 5% and filtration on 0.22 μm PVDF filter, 16.12 mg of ADC were obtained that contained 2.2% of residual example 14. This batch was concentrated on Amicon Ultra-15 and purified on Sephadex G25 to provide 13.16 mg of example 16 as a colorless limpid solution at a concentration of 1.4 mg/mL with a DAR of 2.8 (HRMS), a monomeric purity of 99.7% and a global yield of 43%.

SEC-HRMS: m/z=149398 (naked mAb); m/z=150610 (D1); m/z=151822 (D2); m/z=153035 (D3); m/z=154248 (D4); m/z=155458 (D5); m/z=156672 (D6); m/z=1578881 (D7).

### Synthesis of Examples 17 to 19: glutaryl-Val-GlucoseGln-C52 benzylic amine, NHS ester of glutaryl-Val-GlucoseGln-C52 benzylic amine and Corresponding ADC

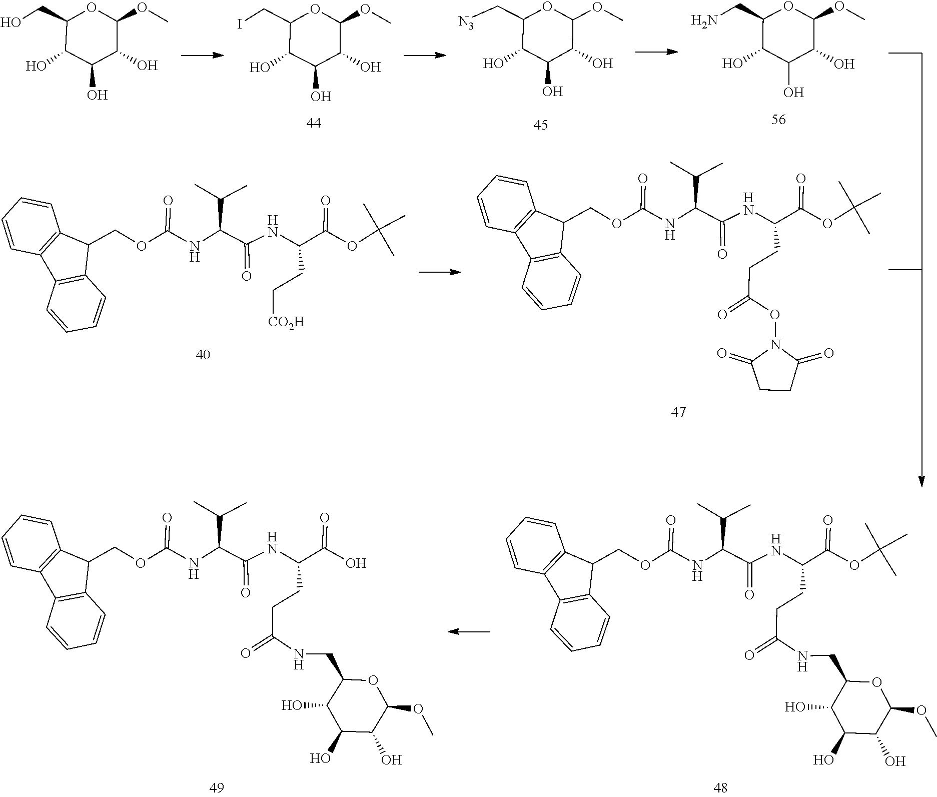

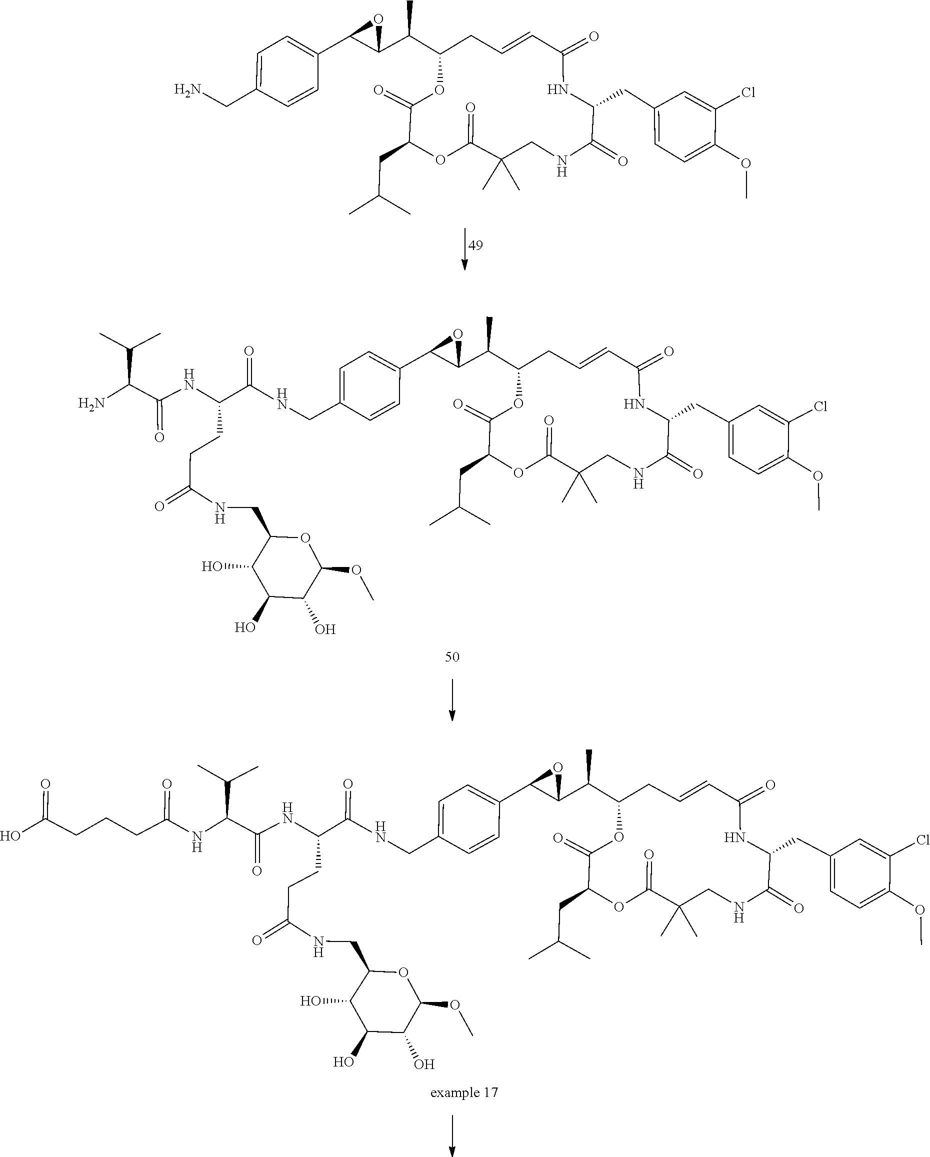

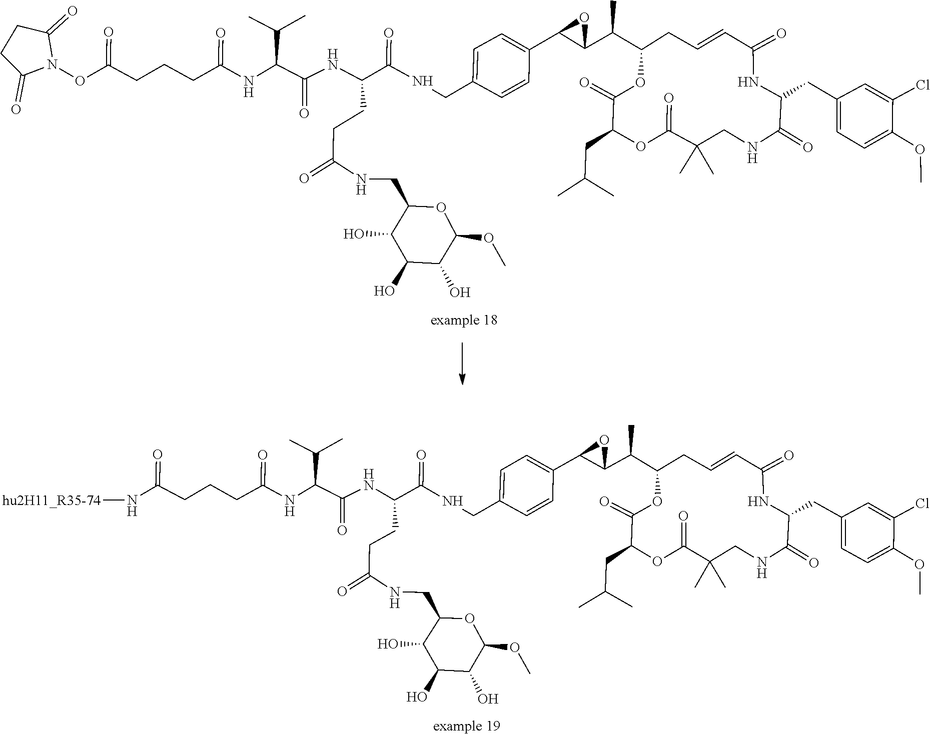

### Compound 44: (2S,3S,4S,5R,6R)-2-(iodomethyl)-6-methoxytetrahydro-2H-pyran-3,4,5-triol

To a solution of methyl β-D-glucopyranoside hemihydrate (CAS number [7000-27-3], 1 g, 4.67 mmol), imidazole (642 mg, 9.34 mmol) and triphenylphosphine (2.47 g, 9.33 mmol) in THF (50 mL) stirred at reflux, was added dropwise a solution of iodine (2.37 g, 9.33 mmol) in THF (10 mL). Reflux was maintained for 3 h. After cooling at 0° C., the reaction medium was filtered, the filtrate was concentrated in vacuo. The mixture was purified by flash chromatography on 150 g of silica gel (gradient DCM/MeOH) to give 1 g of compound 44 (63%).

### Compound 45: (2R,3S,4S,5R,6R)-2-(azidomethyl)-6-methoxytetrahydro-2H-pyran-3,4,5-triol

To a solution of compound 44 (1 g, 3.29 mmol) in DMF (10 mL), under magnetic stirring, was added NaN₃ (322 mg, 4.93 mmol). The reaction medium was heated at 50° C. for 16 h. At this time, after cooling at RT, the medium was concentrated in vacuo and purified by flash chromatography on 80 g of silica gel (gradient elution DCM/MeOH) to give 550 mg of compound 45 (76%).

### Compound 46: (2R,3S,4S,5R,6R)-2-(aminomethyl)-6-methoxytetrahydro-2H-pyran-3,4,5-triol hydrochloride

A solution of compound 45 (550 mg, 2.51 mmol) and triphenylphosphine (1.33 g, 5.02 mmol) in 1,4-dioxane (12 mL) and MeOH (3 mL) was stirred at RT for 30 min. At this time, water (1.5 mL) was added to the reaction medium and stirring at RT was maintained overnight. The crude medium was concentrated in vacuo, then diluted with DCM (10 mL) and aqueous 1N HCl (5 mL), the aqueous phase was extracted with DCM (2×10 mL). The aqueous phase was concentrated in vacuo, then diluted with MeOH and concentrated in vacuo to afford 560 mg of compound 46 (97%).

### Compound 47: (S)-1-tert-butyl 5-(2,5-dioxopyrrolidin-1-yl) 2-((S)-2-((((9H-fluoren-9-yl)methoxy)carbonyl)amino)-3-methylbutanamido)pentanedioate

To a solution of compound 40 (2 g, 3.81 mmol) in THF (100 mL) were added successively, under magnetic stirring, DIEA (1.29 mL, 7.62 mmol), DSC (1.95 g, 7.62 mmol) and DMF (20 mL). The reaction medium was stirred at RT under Ar overnight. After concentration in vacuo, the mixture was purified by flash chromatography on 150 g of silica gel (elution DCM/EtOAc) to give 1.67 g of compound 47 (71%).

RMN ¹H (400 MHz, δ in ppm, DMSO-d6): 0.89 (d, J=6.8 Hz, 3H); 0.91 (d, J=6.8 Hz, 3H); 1.39 (s, 9H); 1.84 to 2.10 (m, 3H); 2.65 to 2.77 (m, 2H); 2.80 (s, 4H); 3.88 (dd, J=7.2 and 8.7 Hz, 1H); 4.18 to 4.31 (m, 4H); 7.32 (m, 2H); 7.41 (m, 3H); 7.73 (d, J=7.6 Hz, 1H); 7.75 (d, J=7.6 Hz, 1H); 7.88 (d, J=7.6 Hz, 2H); 8.31 (broad d, J=7.1 Hz, 1H). LCMS (A): ES m/z=566; m/z=622 [M+H]⁺; m/z=666 [M−H+HCO₂H]⁻; t_{R}=1.49 min.

### Compound 48: (S)-tert-butyl 2-((S)-2-((((9H-fluoren-9-yl)methoxy)carbonyl)amino)-3-methylbutanamido)-5-oxo-5-((((2R,3S,4S,5R,6R)-3,4,5-trihydroxy-6-methoxytetrahydro-2H-pyran-2-yl)methyl)amino)pentanoate

To a solution of compound 47 (500 mg, 804 μmol) in DMF (10 mL) were added, under magnetic stirring, compound 46 (185 mg, 805 μmol) and DIEA (165 μL, 978 μmol). The reaction medium was stirred at RT for 4 h. At this time, 37 mg of compound 46 and 35 μL of DIEA were added to the reaction medium, stirring was maintained for 1 h. Then, the medium was concentrated in vacuo and purified by flash chromatography on 50 g of silica gel (gradient elution DCM/MeOH) to give 480 mg of compound 48 (85%).

RMN ¹H (400 MHz, δ in ppm, DMSO-d6): 0.87 (d, J=6.7 Hz, 3H); 0.89 (d, J=6.7 Hz, 3H); 1.38 (s, 9H); 1.70 to 2.04 (m. 3H); 2.19 (t, J=7.9 Hz, 2H); 2.86 to 2.98 (m, 2H); 3.01 to 3.16 (m, 3H); 3.35 (s, 3H); 3.52 (m, 1H); 3.92 (dd, J=7.2 and 8.4 Hz, 1H); 3.99 (d, J=7.6 Hz, 1H); 4.09 (m, 1H); 4.16 to 4.36 (m, 3H); 4.95 (broad d, J=4.6 Hz, 1H); 4.99 (d, J=5.1 Hz, 1H); 5.03 (d, J=4.9 Hz, 1H); 7.27 to 7.37 (m, 3H); 7.41 (t, J=7.6 Hz, 2H); 7.75 (split d, J=7.0 Hz, 2H); 7.85 (broad m, 1H); 7.89 (d, J=7.6 Hz, 2H); 8.23 (broad d, J=7.1 Hz, 1H). LCMS (A): ES m/z=700 [M+H]⁺; m/z=744 [M−H+HCO₂H]⁻; t_{R}=1.22 min.

### Compound 49: (S)-2-((S)-2-((((9H-fluoren-9-yl)methoxy)carbonyl)amino)-3-methylbutanamido)-5-oxo-5-((((2R,3S,4S,5R,6R)-3,4,5-trihydroxy-6-methoxytetrahydro-2H-pyran-2-yl)methyl)amino)pentanoic acid

To compound 48 (480 mg, 686 μmol), under magnetic stirring, was added a 4M solution of HCl in 1,4-dioxane (30 mL). The reaction medium was stirred at RT for 4 h. At this time, the medium was concentrated in vacuo and purified by flash chromatography on 30 g of silica gel (elution DCM/MeOH/H₂O) to give 260 mg of compound 49 (59%).

RMN ¹H (400 MHz, δ in ppm, DMSO-d6): 0.83 (d, J=6.6 Hz, 3H); 0.85 (d, J=6.6 Hz, 3H); 1.69 to 1.90 (m, 2H); 2.01 to 2.15 (m, 3H); 2.86 to 2.97 (m, 2H); 2.99 to 3.16 (m, 3H); 3.35 (s, 3H); 3.51 (m, 1H); 3.82 (m, 2H); 3.99 (d, J=7.8 Hz, 1H); 4.20 to 4.33 (m, 3H); 4.95 (broad d, J=3.7 Hz, 1H); 5.03 (d, J=4.9 Hz, 1H); 5.06 (broad d, J=4.6 Hz, 1H); 7.33 (m, 2H); 7.40 (t, J=7.6 Hz, 2H); 7.52 (broad d, J=6.4 Hz, 1H); 7.61 (broad d, J=9.3 Hz, 1H); 7.73 (d, J=7.6 Hz, 1H); 7.76 (d, J=7.6 Hz, 1H); 7.89 (d, J=7.6 Hz, 2H); 8.10 (broad m, 1H). LCMS (A): ES m/z=642 [M−H]⁻; m/z=644 [M+H]⁺; t_{R}=1.01 min.

### Compound 50: (S)-2-((S)-2-amino-3-methylbutanamido)-N₁-(4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)-N₅-(((2R,3S,4S,5R,6R)-3,4,5-trihydroxy-6-methoxytetrahydro-2H-pyran-2-yl)methyl)pentanediamide

To a solution of (E)-(3S,10R,16S)-16-{(S)-1-[(2R,3R)-3-(4-aminomethyl-phenyl)-oxiranyl]-ethyl}-10-(3-chloro-4-methoxy-benzyl)-3-isobutyl-6,6-dimethyl-1,4-dioxa-8,11-diaza-cyclohexadec-13-ene-2,5,9,12-tetraone (the synthesis of which was described in WO2011001052, compound 77, 110 mg, 157 μmol) in DMF (6 mL) were added, under magnetic stirring, compound 49 (122 mg, 189 μmol), HOBt (34 mg, 252 μmol) and EDC (43 μL, 236 μmol). The reaction medium was stirred at RT overnight. Then, 25 mg of compound 49 and 25 μL of EDC were added to the medium, stirring was maintained for 1 h. At this time, piperidine (160 μL, 1.58 mmol) was added to the medium. After 2 h, the medium was concentrated in vacuo and purified by flash chromatography on 20 g of silica gel (gradient elution DCM/MeOH) to give 78 mg of compound 50 (45%).

RMN ¹H (500 MHz, δ in ppm, DMSO-d6): 0.76 (d, J=6.9 Hz, 3H); 0.79 (d, J=6.3 Hz, 6H); 0.87 (d, J=6.9 Hz, 3H); 1.00 (s, 3H); 1.04 (d, J=6.6 Hz, 3H); 1.12 (s, 3H); 1.30 (m, 1H); 1.50 to 1.98 (m, 8H); 2.12 (m, 2H); 2.26 (m, 1H); 2.68 (m, 2H); 2.86 to 3.14 (m, 9H); 3.32 (partially masked m, 1H); 3.38 (s, 3H); 3.52 (m, 1H); 3.81 (s, 3H); 3.87 (s, 1H); 4.01 (d, J=7.4 Hz, 1H); 4.20 to 4.36 (m, 4H); 4.90 (m, 1H); 4.98 (broad d, J=3.6 Hz, 1H); 5.02 (d, J=4.9 Hz, 1H); 5.06 (d, J=4.7 Hz, 1H); 5.10 (m, 1H); 5.79 (d, J=15.6 Hz, 1H); 6.47 (m, 1H); 7.05 (d, J=8.5 Hz, 1H); 7.17 (broad d, J=8.5 Hz, 1H); 7.21 to 7.26 (m, 5H); 7.29 (broad s, 1H); 7.90 (m, 1H); 8.08 (m large, 1H); 8.37 (d, J=8.0 Hz, 1H); 8.48 (m, 1H). LCMS (A): ES m/z=551 [M+2H]²⁺; m/z=1099 [M−H]⁻; m/z=1101 [M+H]⁺; m/z=1145 [M−H+HCO₂H]⁻; t_{R}=0.9 min.

### Example 17: 5-(((S)-1-(((S)-1-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)amino)-1,5-dioxo-5-((((2R,3S,4S,5R,6R)-3,4,5-trihydroxy-6-methoxytetrahydro-2H-pyran-2-yl)methyl)amino)pentan-2-yl)amino)-3-methyl-1-oxobutan-2-yl)amino)-5-oxopentanoic acid

To a solution of compound 50 (77 mg, 69.9 μmol) in DMF (4 mL) was added, under magnetic stirring, glutaric anhydride (14 mg, 125.8 μmol). The reaction medium was stirred at RT for 2 h. Then, the reaction medium was concentrated in vacuo and purified by flash chromatography on 10 g of silica gel (gradient elution DCM/MeOH) to give 54 mg of example 17 (64%).

RMN ¹H (500 MHz, δ in ppm, DMSO-d6): 0.80 (d, J=6.0 Hz, 6H); 0.86 (d, J=6.6 Hz, 3H); 0.88 (d, J=6.6 Hz, 3H); 0.99 (s, 3H); 1.03 (d, J=6.6 Hz, 3H); 1.12 (s, 3H); 1.34 (m, 1H); 1.53 to 1.85 (m, 6H); 1.90 to 2.33 (m, 9H); 2.65 to 2.76 (m, 2H); 2.87 to 3.14 (m, 8H); 3.32 (partially masked m, 1H); 3.35 (s, 3H); 3.53 (m, 1H); 3.81 (s, 3H); 3.87 (d, J=1.9 Hz, 1H); 4.00 (d, J=7.9 Hz, 1H); 4.03 to 4.26 (m, 4H); 4.33 (m, 1H); 4.93 (m, 1H); 5.02 (broad m, 3H); 5.10 (m, 1H); 5.80 (d, J=15.3 Hz, 1H); 6.46 (ddd, J=3.5, 11.0 and 15.3 Hz, 1H); 7.05 (d, J=8.5 Hz, 1H); 7.18 (dd, J=2.2 and 8.5 Hz, 1H); 7.22 (d, J=8.2 Hz, 2H); 7.26 (d, J=8.2 Hz, 2H); 7.30 (m, 2H); 7.98 to 8.65 (broad m, 5H). LCMS (A): ES m/z=608 [M+2H]²⁺; m/z=1213 [M−H]⁻; m/z=1215 [M+H]⁺; t_{R}=1.15 min.

### Example 18: 2,5-dioxopyrrolidin-1-yl 5-(((S)-1-(((S)-1-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)amino)-1,5-dioxo-5-((((2R,3S,4S,5R,6R)-3,4,5-trihydroxy-6-methoxytetrahydro-2H-pyran-2-yl)methyl)amino)pentan-2-yl)amino)-3-methyl-1-oxobutan-2-yl)amino)-5-oxopentanoate

Example 17 (45 mg, 37 μmol) was diluted in toluene and concentrated in vacuo. Then, to a solution of example 17 in THF (5 mL), DCM (2 mL) and DMF (1 mL) were added, under magnetic stirring, DSC (11.4 mg, 44.4 μmol) and DIEA (19 μL, 112.6 μmol). The reaction medium was stirred at RT under Ar for 3 h. Then, DMF and DSC were added to the medium until completion of the reaction. After 3 h, the medium was diluted with water (5 mL), and extracted with MeTHF (3×10 mL). The combined organic phases were dried over MgSO₄, filtered and concentrated in vacuo. The mixture was purified by flash chromatography on 4 g of silica gel (gradient elution DCM/iPrOH) to give 21 mg of example 18 (43%).

RMN ¹H (500 MHz, δ in ppm, DMSO-d6): 0.78 (d, J=6.0 Hz, 6H); 0.83 (d, J=6.9 Hz, 3H); 0.85 (d, J=6.9 Hz, 3H); 1.00 (s, 3H); 1.04 (d, J=6.3 Hz, 3H); 1.12 (s, 3H); 1.30 (m, 1H); 1.52 to 1.63 (m, 2H); 1.75 to 2.02 (m, 6H); 2.09 to 2.34 (m, 5H); 2.68 (m, 4H); 2.81 (s, 4H); 2.88 to 3.18 (m, 7H); 3.32 (partially masked m, 1H); 3.38 (s, 3H); 3.51 (m, 1H); 3.81 (s, 3H); 3.88 (s, 1H); 4.01 (d, J=7.7 Hz, 1H); 4.16 (dd, J=7.1 and 7.7 Hz, 1H); 4.19 to 4.35 (m, 5H); 4.90 (m, 1H); 4.97 (d, J=4.1 Hz, 1H); 5.01 (d, J=4.9 Hz, 1H); 5.06 (d, J=4.9 Hz, 1H); 5.10 (m, 1H); 5.79 (d, J=15.3 Hz, 1H); 6.47 (m, 1H); 7.05 (d, J=8.5 Hz, 1H); 7.17 (d large, J=8.5 Hz, 1H); 7.20 to 7.26 (m, 5H); 7.28 (broad s, 1H); 7.86 (broad t, J=5.6 Hz, 1H); 7.91 (d, J=7.9 Hz, 1H); 8.08 (d, J=7.4 Hz, 1H); 8.33 (t, J=5.8 Hz, 1H); 8.38 (d, J=8.2 Hz, 1H). LCMS (A): ES m/z=1310 [M−H]⁻; m/z=1356 [M−H+HCO₂H]⁻; m/z=1312 [M+H]⁺; m/z=656.5 [M+2H]²⁺; t_{R}=1.2 min.

### Example 19: hu2H11_R35-74-Ex18

The general method described previously was used for the preparation of example 19. 29.02 mg of hu2H11_R35-74 were reacted with 116.6 μL of a 10 mM solution of example 18 in DMA (6 eq.) for 2 h 30. After purification on Superdex 200 μg in buffer B pH 6.5+10% NMP, concentration on Amicon Ultra-15, dilution in buffer B pH 6.5 to a final concentration of NMP at 5% and filtration on 0.22 μm PVDF filter, 23.1 mg of example 19 were obtained as a colorless limpid solution at a concentration of 2.2 mg/mL with a DAR of 2.8 (HRMS), a monomeric purity of 99.9% and a global yield of 78%. Free-drug level was above the threshold of 1%: the ADC was concentrated on Amicon Ultra-15, purified on Sephadex G25 in buffer B pH 6.5+5% NMP and filtrated on 0.22 μm PVDF filter to provide 17.25 mg of example 19 as a colorless limpid solution at a concentration of 1.50 mg/mL with a DAR of 2.55 (HRMS), a monomeric purity of 99.7% and a global yield of 59%.

SEC-HRMS: m/z=149405 (naked mAb); m/z=150602 (D1); m/z=151800 (D2); m/z=152997 (D3); m/z=154196 (D4); m/z=155393 (D5); m/z=156589 (D6); m/z=157791 (D7).

### Synthesis of Example 20: glutaryl-Val-GlucamineGln-C52 benzylic amine

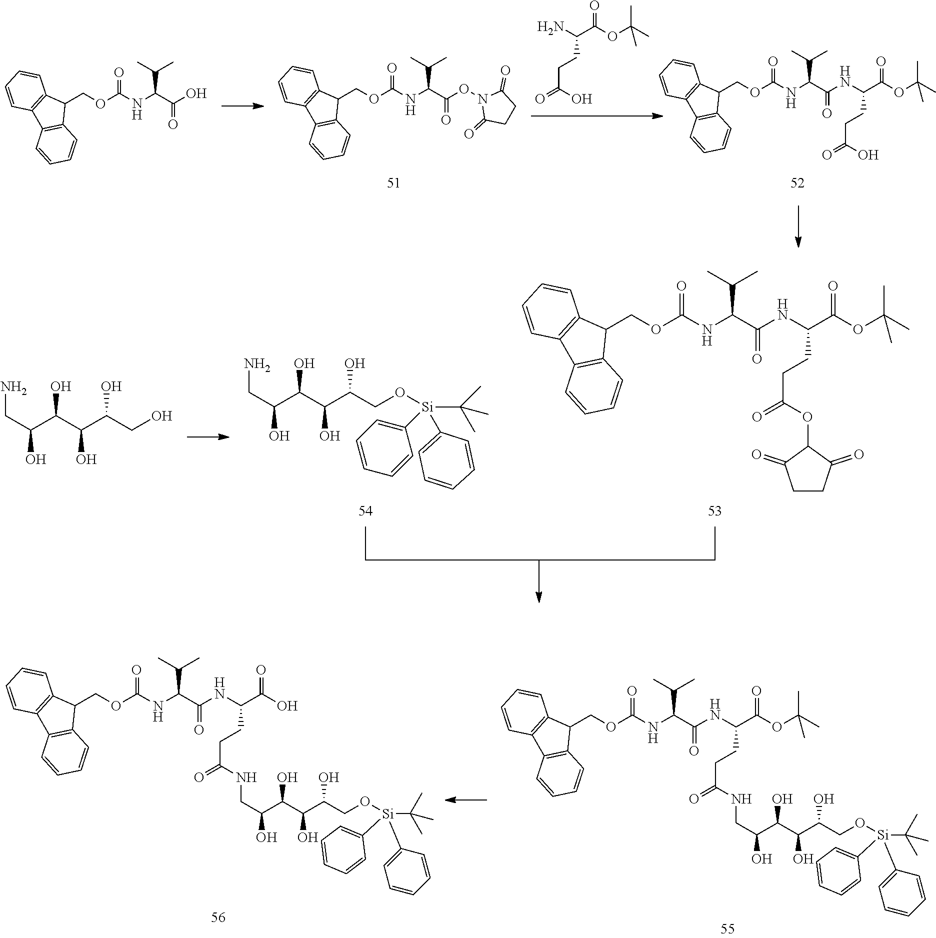

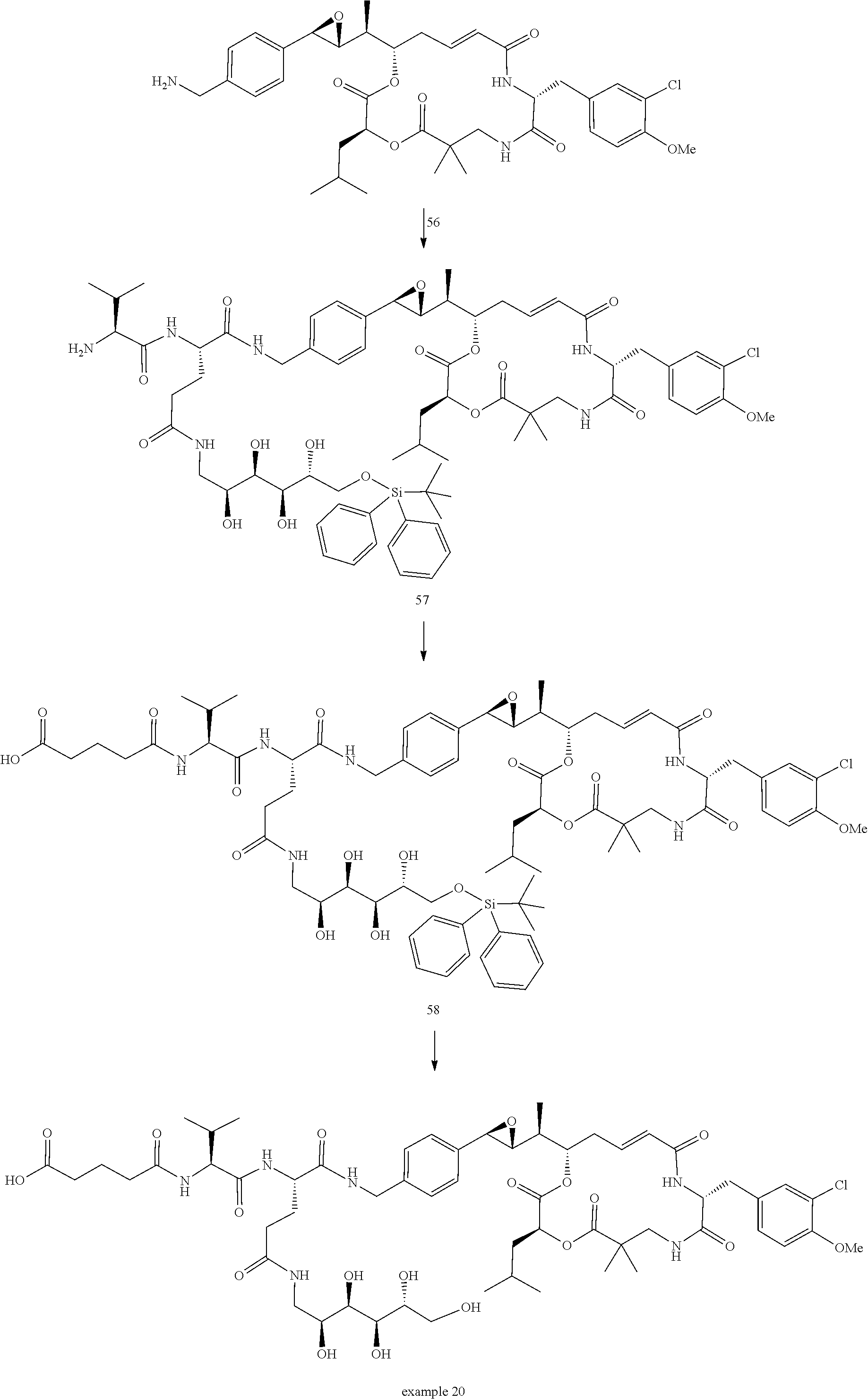

### Compound 51: (S)-2,5-dioxopyrrolidin-1-yl 2-((((9H-fluoren-9-yl)methoxy)carbonyl)amino)-3-methylbutanoate

To a solution of Fmoc-Val-OH (CAS number [68858-20-8], 5 g, 14.44 mmol) in THF (40 mL) were added NHS (1.71 g, 14.44 mmol) and DCC (2.98 g, 14.44 mmol). The reaction medium was stirred for 5 h at RT, then 0.2 equivalents of NHS and DCC were added and stirring was carried on for 4 h at RT. The medium was filtered, the solid washed twice with THF (2×25 mL) and the filtrate concentrated in vacuo to give 6.5 g of compound 51 as a white meringue (quant.).

RMN ¹H (400 MHz, δ in ppm, DMSO-d6): 1.03 (d, J=6.9 Hz, 6H); 2.20 (m, 1H); 2.81 (s, 4H); 4.18 to 4.39 (m, 4H); 7.32 (m, 2H); 7.42 (m, 2H); 7.74 (m, 2H); 7.90 (d, J=7.6 Hz, 2H); 8.12 (d, J=8.5 Hz, 1H).

### Compound 52: (S)-4-((S)-2-((((9H-fluoren-9-yl)methoxy)carbonyl)amino)-3-methylbutanamido)-5-(tert-butoxy)-5-oxopentanoic acid

To a solution of H-Glu-OtBu (CAS number [45120-30-7], 3.14 g, 15.16 mmol) and sodium bicarbonate (1.40 g, 16.46 mmol) in H₂O (60 mL) was added a solution of compound 51 (6.3 g, 14.43 mmol) in THF (240 mL). The reaction medium was stirred for 16 h at RT and concentrated in vacuo. The crude product was diluted in H₂O (700 mL) forming a gel that was stirred for 15 min then extracted twice with Et₂O (200 mL). The aqueous suspension was acidified to pH 3 with 5 N HCl and extracted with DCM (4×200 mL). The combined organic phases were dried over MgSO₄, filtered and concentrated in vacuo to give 7.98 g of compound 52 as a white meringue (quant.).

RMN ¹H (400 MHz, δ in ppm, DMSO-d6): 0.87 (d, J=6.9 Hz, 3H); 0.90 (d, J=6.9 Hz, 3H); 1.38 (s, 9H); 1.77 (m, 1H); 1.91 (m, 1H); 1.98 (m, 1H); 2.28 (m, 2H); 3.90 (dd, J=7.1 and 9.1 Hz, 1H); 4.10 to 4.37 (m, 4H); 7.32 (m, 2H); 7.38 (d, J=9.1 Hz, 1H); 7.41 (m, 2H); 7.73 (m, 2H); 7.89 (d, J=7.6 Hz, 2H); 8.20 (d, J=7.5 Hz, 1H); 12.21 (broad m, 1H).

### Compound 53: (S)-1-tert-butyl 5-(2,5-dioxopyrrolidin-1-yl) 2-((S)-2-((((9H-fluoren-9-yl)methoxy) carbonyl)amino)-3-methylbutanamido)pentanedioate

To a solution of compound 52 (500 mg, 953.1 μmol) in THF (30 mL) were added DIEA (320 μL, 1.9 mmol), DSC (498.3 mg, 1.91 mmol) and DMF (6 mL). The reaction medium was stirred at RT overnight then 30 mg of DSC were added to the medium and stirring was carried on 24 h. The reaction medium was concentrated in vacuo, co-evaporated twice with toluene and purified by flash chromatography on 50 g of silica gel (gradient elution DCM/iPrOH) to give 506 mg of compound 53 as a white oil (85%).

RMN ¹H (400 MHz, δ in ppm, DMSO-d6): 0.89 (d, J=6.9 Hz, 3H); 0.91 (d, J=6.9 Hz, 3H); 1.39 (s, 9H); 1.75 to 2.11 (m, 3H); 2.73 (m, 2H); 2.80 (s, 4H); 3.89 (dd, J=7.3 and 9.1 Hz, 1H); 4.20 to 4.38 (m, 4H); 7.32 (m, 2H); 7.42 (m, 3H); 7.75 (m, 2H); 7.80 (m, 2H); 8.31 (d, J=7.5 Hz, 1H).

### Compound 54: (2S,3R,4R,5R)-1-amino-6-((tert-butyldiphenylsilyl)oxy)hexane-2,3,4,5-tetraol

A suspension of D-glucamine (CAS number [488-43-7], 200 mg, 1.10 mmol) in DMF (2 mL) was cooled at 0° C. then were added imidazole (82.0 mg, 1.19 mmol) and tert-butylchlorodiphenylsilane (334.4 mg, 1.19 mmol). The reaction medium was stirred for 1 h at 0° C., quenched with H₂O (20 mL) and stirring carried on 10 min. The medium was extracted with EtOAc (3×50 mL), the combined organic phases were dried over MgSO₄, filtered, concentrated in vacuo, co-evaporated twice with toluene and purified by reverse phase flash chromatography on 31 g of C18-modified silica gel (gradient elution H₂O/MeCN) to give 218 mg of compound 54 as a white foam (47%).

RMN ¹H (400 MHz, δ in ppm, DMSO-d6): 1.00 (s, 9H); 2.62 (dd, J=6.3 and 12.7 Hz, 1H); 2.74 (dd, J=4.5 and 12.7 Hz, 1H); 3.40 (partially masked m, 1H); 3.49 (m, 1H); 3.55 to 3.72 (m, 3H); 3.88 (m, 1H); 4.48 (broad m, 4H); 7.42 (m, 6H); 7.69 (m, 4H).

### Compound 55: (S)-tert-butyl 2-((S)-2-((((9H-fluoren-9-yl)methoxy)carbonyl)amino)-3-methylbutanamido)-5-(((2S,3R,4R,5R)-6-((tert-butyldiphenylsilyl)oxy)-2,3,4,5-tetrahydroxyhexyl) amino)-5-oxopentanoate

To a solution of compound 53 (300 mg, 482.6 μmol) in DMF (2 mL) was added a solution of compound 54 (214.6 mg, 511.5 μmol) in DMF (3 mL). The reaction medium was stirred for 1 h at RT, concentrated in vacuo, co-evaporated with toluene (3×) and purified by flash chromatography on 25 g of silica gel (gradient elution DCM/MeOH/H₂O) to give 270 mg of compound 55 as a white foam (60%).

RMN ¹H (400 MHz, δ in ppm, DMSO-d6): 0.87 (d, J=6.9 Hz, 3H); 0.90 (d, J=6.9 Hz, 3H); 0.99 (s, 9H); 1.38 (s, 9H); 1.80 (m, 1H); 1.90 (m, 1H); 1.98 (m, 1H); 2.17 (t, J=7.7 Hz, 2H); 3.03 (m, 1H); 3.26 (m, 1H); 3.49 (m, 1H); 3.52 to 3.60 (m, 4H); 3.83 (m, 1H); 3.92 (dd, J=7.3 and 9.2 Hz, 1H); 4.08 (m, 1H); 4.18 to 4.31 (m, 4H); 4.33 (d, J=6.4 Hz, 1H); 4.63 (d, J=5.8 Hz, 1H); 4.78 (d, J=4.7 Hz, 1H); 7.31 (m, 2H); 7.36 (d, J=9.2 Hz, 1H); 7.41 (m, 8H); 7.67 (m, 4H); 7.69 to 7.76 (m, 3H); 7.89 (m, 2H); 8.23 (d, J=7.5 Hz, 1H).

### Compound 56: (S)-2-((S)-2-((((9H-fluoren-9-yl)methoxy)carbonyl)amino)-3-methylbutanamido)-5-(((2S,3R,4R,5R)-6-((tert-butyldiphenylsilyl)oxy)-2,3,4,5-tetrahydroxyhexyl)amino)-5-oxopentanoic acid

Compound 55 was diluted in DCM forming a gel that was cooled at 0° C. before slowly adding, at 0° C., a 1:1 mixture of DCM/TFA. The reaction medium was stirred for 30 min at 0° C. then 5 h at RT, concentrated in vacuo and co-evaporated with toluene (3×) to give a white solid that was grinded up in iPr₂O, filtered, washed twice with Et₂O (5 mL), dried and finally purified by flash chromatography on 15 g of silica gel (gradient elution DCM/MeOH/H₂O) to give 149 mg of compound 56 as a white solid (59%).

RMN ¹H (400 MHz, δ in ppm, DMSO-d6): 0.84 (d, J=6.8 Hz, 3H); 0.86 (d, J=6.8 Hz, 3H); 0.99 (s, 9H); 1.82 (m, 1H); 1.90 (m, 1H); 2.03 to 2.14 (m, 3H); 3.00 (m, 1H); 3.25 (m, 1H); 3.48 (m, 1H); 3.65 (m, 4H); 3.82 to 3.92 (m, 3H); 4.20 to 4.37 (m, 3H); 4.40 (d, J=6.0 Hz, 1H); 4.45 (d, J=6.0 Hz, 1H); 4.80 (m, 1H); 5.12 (m, 1H); 7.32 (m, 2H); 7.41 (m, 8H); 7.55 (m, 2H); 7.65 to 7.82 (m, 7H); 7.89 (d, J=8.0 Hz, 2H). LCMS (A): m/z=868 [M−H]⁻; m/z=870 [M+H]⁺; t_{R}=1.55 min.

### Compound 57: (S)-2-((S)-2-amino-3-methylbutanamido)-N₅-((2S,3R,4R,5R)-6-((tert-butyldiphenylsilyl)oxy)-2,3,4,5-tetrahydroxyhexyl)-N₁-(4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclo-hexadec-13-en-16-yl)ethyl)oxiran-2-yl)benzyl)-pentanediamide

To a solution of compound 56 (67.3 mg, 77.3 μmol) and (3S,10R,16S,E)-16-((S)-1-((2R,3R)-3-(4-(aminomethyl)phenyl)oxiran-2-yl)ethyl)-10-(3-chloro-4-methoxybenzyl)-3-isobutyl-6,6-dimethyl-1,4-dioxa-8,11-diazacyclohexadec-13-ene-2,5,9,12-tetraone (the synthesis of which was described in WO2011001052, compound 77, 45 mg, 64.5 μmol) in DMF and DCM (10 mL) were added HOBt (11.5 mg, 85.1 μmol) and EDC (11.4 mg, 70.9 μmol). The reaction medium was stirred for 3 h at RT then quenched with H₂O (15 mL) and stirring was carried on 10 min. The aqueous phase was extracted with DCM (3×20 mL), the combined organic phases were dried over MgSO₄, filtered, concentrated in vacuo and purified by flash chromatography on 5 g of silica gel (gradient elution DCM/MeOH) to give 64 mg of Fmoc-protected dipeptide-cryptophycin intermediate as a white solid (64%).

RMN ¹H (500 MHz, δ in ppm, DMSO-d6): 0.76 (d, J=6.8 Hz, 6H); 0.82 (d, J=7.0 Hz, 3H); 0.86 (d, J=7.0 Hz, 3H); 0.99 (s, 12H); 1.04 (d, J=7.1 Hz, 3H); 1.10 (s, 3H); 1.30 (m, 1H); 1.55 (m, 2H); 1.80 (m, 2H); 1.90 (m, 1H); 2.00 (m, 1H); 2.14 (m, 2H); 2.26 (m, 1H); 2.68 (m, 2H); 2.92 to 3.08 (m, 4H); 3.22 to 3.38 (partially masked m, 2H); 3.49 (m, 1H); 3.58 to 3.69 (m, 4H); 3.81 (s, 3H); 3.82 (m, 1H); 3.87 (d, J=2.0 Hz, 1H); 3.91 (m, 1H); 4.18 to 4.38 (m, 9H); 4.68 (m, 1H); 4.82 (m, 1H); 4.90 (dd, J=3.8 and 9.8 Hz, 1H); 5.10 (m, 1H); 5.79 (broad d, J=16.0 Hz, 1H); 6.47 (ddd, J=4.7, 10.5 and 16.0 Hz, 1H); 7.05 (d, J=8.0 Hz, 1H); 7.17 (dd, J=2.5 and 8.0 Hz, 1H); 7.20 to 7.27 (m, 5H); 7.29 (d, J=2.3 Hz, 1H); 7.31 (t, J=7.8 Hz, 2H); 7.34 to 7.48 (m, 7H); 7.65 to 7.79 (m, 9H); 7.90 (d, J=7.8 Hz, 2H); 8.10 (d, J=8.3 Hz, 1H); 8.38 (d, J=8.0 Hz, 1H); 8.41 (t, J=6.5 Hz, 1H). LCMS (A): ES m/z=775 [M+2H]²⁺; m/z=1549 [M+H]⁺; m/z=1593 [M−H+HCO₂H]⁻; t_{R}=1.78 min.

To a solution of this intermediate (62 mg, 40.0 μmol) in DCM (8 mL) was added piperidine (59.8 μL, 600.0 μmol). The reaction medium was stirred for 4 h at RT, concentrated in vacuo and purified by flash chromatography on 5 g of silica gel (gradient elution DCM/MeOH/H₂O) to give 49 mg of compound 57 as a white lacquer (92%).

### Compound 58: (6R,7R,8R,9S,15S,18S)-15-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)-6,7,8,9-tetrahydroxy-18-isopropyl-2,2-dimethyl-12,17,20-trioxo-3,3-diphenyl-4-oxa-11,16,19-triaza-3-silatetracosan-24-oic acid

To a solution of compound 57 (23 mg, 17.3 μmol) in DCM (3 mL) was added a solution of glutaric anhydride (2.22 mg, 19.1 μmol) in DCM (2 mL). The reaction medium was stirred for 3 h at RT, parly concentrated in vacuo (down to 2 mL) and purified by flash chromatography on 5 g of silica gel (gradient elution DCM/MeOH/H₂O) to give 17 mg of compound 58 as a white lacquer (68%).

RMN ¹H (500 MHz, δ in ppm, DMSO-d6): 0.80 (split d, J=6.8 Hz, 6H); 0.6 (split d, J=7.0 Hz, 6H); 0.99 (s, 12H); 1.04 (d, J=7.1 Hz, 3H); 1.11 (s, 3H); 1.32 (m, 1H); 1.52 to 1.60 (m, 2H); 1.72 to 2.00 (m, 7H); 2.08 (m, 1H); 2.13 (m, 1H); 2.20 to 2.37 (m, 3H); 2.69 (m, 2H); 2.92 to 3.07 (m, 4H); 3.20 (m, 1H); 3.30 (masked m, 1H); 3.45 (m, 1H); 3.55 to 3.70 (m, 4H); 3.80 (s, 3H); 3.83 (m, 1H); 3.87 (d, J=2.3 Hz, 1H); 4.07 to 5.00 (m, 11H); 5.10 (m, 1H); 5.80 (dd, J=2.0 and 15.8 Hz, 1H); 6.47 (m, 1H); 7.04 (d, J=8.6 Hz, 1H); 7.18 (dd, J=2.4 and 8.6 Hz, 1H); 7.20 to 7.28 (m, 5H); 7.29 (d, J=2.3 Hz, 1H); 7.41 (m, 6H); 7.68 (m, 4H); 8.05 (d, J=7.9 Hz, 1H; 8.23 (t, J=6.5 Hz, 1H); 8.30 to 8.60 (m, 3H). LCMS (A): ES m/z=721 [M+2H]²⁺; m/z=1439 [M−H]⁻; m/z=1441 [M+H]⁺; t_{R}=1.52 min.

### Example 20: 5-(((S)-1-(((S)-1-((4-((2R,3R)-3-((S)-1-((3S,10R,16S,E)-10-(3-chloro-4-methoxy-benzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)amino)-1,5-dioxo-5-(((2S,3R,4R,5R)-2,3,4,5,6-pentahydroxyhexyl)-amino)pentan-2-yl)amino)-3-methyl-1-oxobutan-2-yl)amino)-5-oxopentanoic acid

A solution of compound 57 (46 mg, 31.9 μmol) in THF (3 mL) was cooled at 0° C. then was added, dropwise at 0° C., a 1 M TBAF solution in THF (35.1 μL, 35.1 μmol). The reaction medium was stirred for 2 h at 0° C., quenched with H₂O (100 μL), stirred for 10 min and extracted with DCM (5 mL). The organic phase was dried over MgSO₄, filtered and concentrated in vacuo: LCMS analysis showed the presence of residual starting material. The crude product was dissolved in DCM (3 mL), the solution cooled at 0° C. before adding, dropwise at 0° C., a 1 M TBAF solution in THF (35.1 μL, 35.1 μmol). The reaction medium was stirred for 3 h at 0° C., quenched with H₂O (100 μL), stirred for 10 min and extracted with DCM (20 mL). The organic phase was dried over MgSO₄, filtered, concentrated in vacuo and purified by flash chromatography on 5 g of silica gel (gradient elution DCM/MeOH/H₂O to MeOH) then by reverse phase flash chromatography on 3 g of C18-modified silica gel (gradient elution H₂O/MeCN) to give 17 mg of example 20 as a white lacquer (44%).

RMN ¹H (500 MHz, δ in ppm, DMSO-d6): 0.80 (split d, J=6.8 Hz, 6H); 0.6 (split d, J=7.0 Hz, 6H); 0.99 (s, 12H); 1.04 (d, J=7.1 Hz, 3H); 1.11 (s, 3H); 1.32 (m, 1H); 1.52 to 1.60 (m, 2H); 1.72 to 2.00 (m, 7H); 2.08 (m, 1H); 2.13 (m, 1H); 2.20 to 2.37 (m, 3H); 2.69 (m, 2H); 2.92 to 3.07 (m, 4H); 3.20 (m, 1H); 3.30 (masked m, 1H); 3.45 (m, 1H); 3.55 to 3.70 (m, 4H); 3.80 (s, 3H); 3.83 (m, 1H); 3.87 (d, J=2.3 Hz, 1H); 4.07 to 5.00 (m, 11H); 5.10 (m, 1H); 5.80 (dd, J=2.0 and 15.8 Hz, 1H); 6.47 (m, 1H); 7.04 (d, J=8.6 Hz, 1H); 7.18 (dd, J=2.4 and 8.6 Hz, 1H); 7.20 to 7.28 (m, 5H); 7.29 (d, J=2.3 Hz, 1H); 7.41 (m, 6H); 7.68 (m, 4H); 8.05 (d, J=7.9 Hz, 1H); 8.23 (t, J=6.5 Hz, 1H); 8.30 to 8.60 (m, 3H). LCMS (A): ES m/z=602 [M+2H]²⁺; m/z=1201 [M−H]⁻; m/z=1203 [M+H]⁺; t_{R}=1.1 min.

### Synthesis of Examples 21 to 23: glutaryl-Val-PEG4Lys-C52 benzylic amine, NHS ester of glutaryl-Val-PEG4Lys-C52 benzylic amine and Corresponding ADC

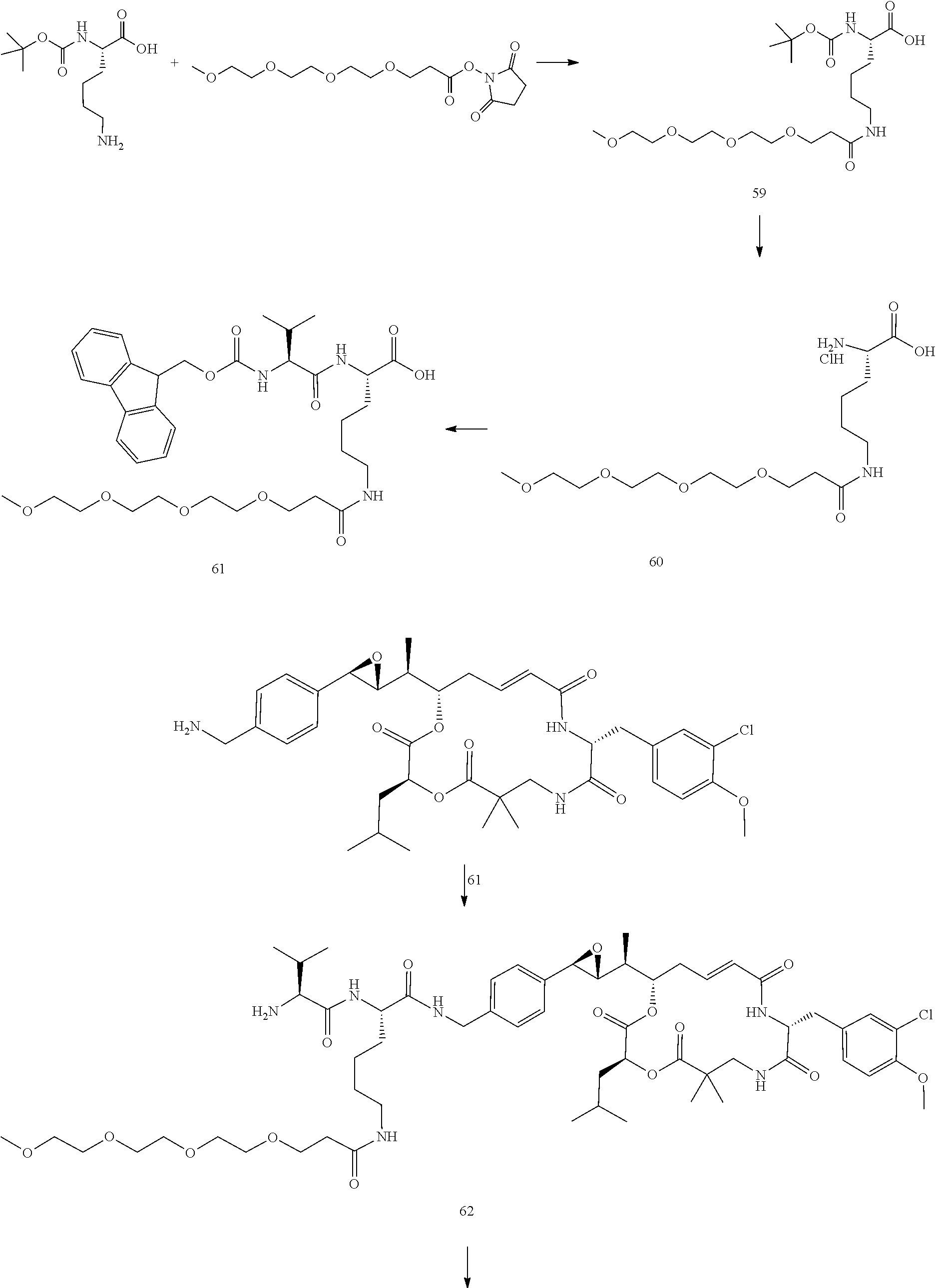

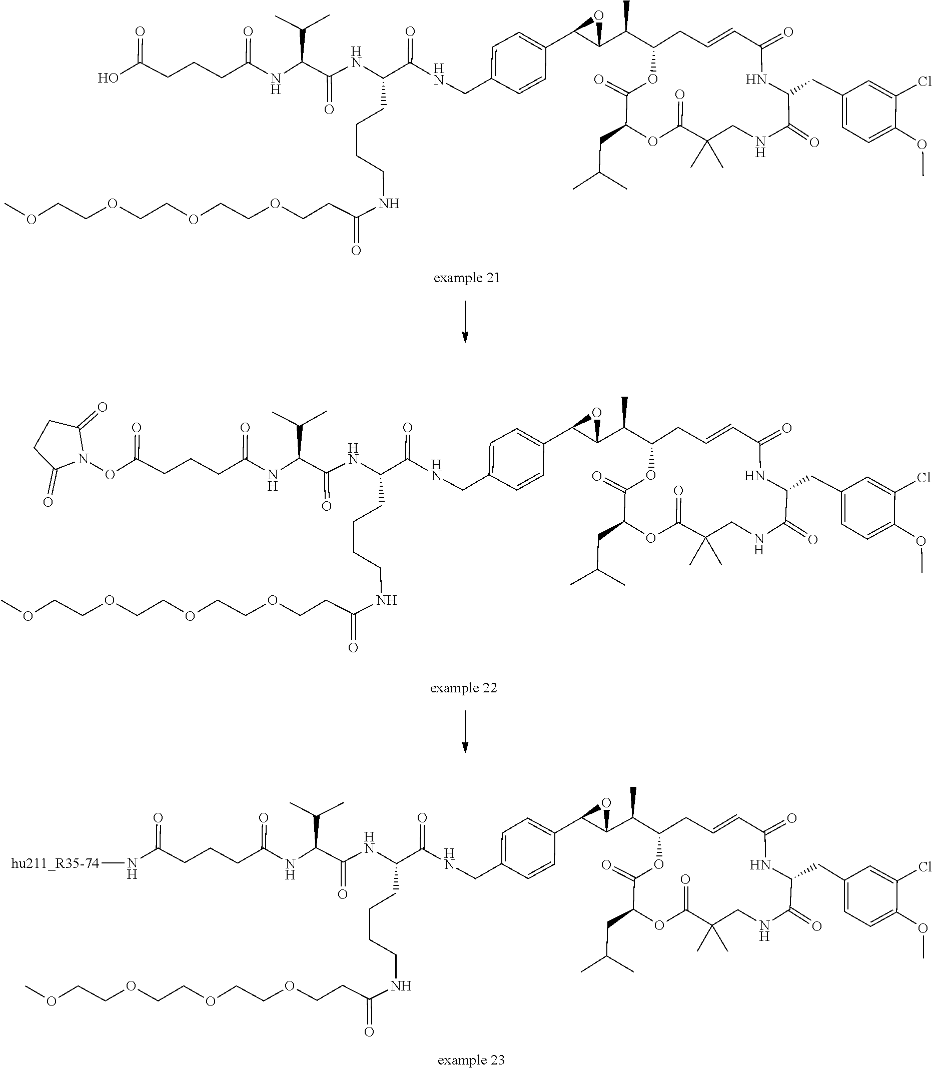

### Compound 59: (S)-20-((tert-butoxycarbonyl)amino)-14-oxo-2,5,8,11-tetraoxa-15-azahenicosan-21-oic acid

To a solution of (S)-6-amino-2-((tert-butoxycarbonyl)amino)hexanoic acid (CAS number [13734-28-6], 73.9 mg, 300 μmol) in THF (300 μL) were added, under magnetic stirring, 2,5-dioxopyrrolidin-1-yl 2,5,8,11-tetraoxatetradecan-14-oate (CAS number [622405-78-1], 100 mg, 300 μmol), DIEA (53.3 μL, 300 μmol) and H₂O (100 μL). The reaction medium was stirred at RT overnight then concentrated in vacuo. The crude medium was purified by flash chromatography on 5 g of C18-grafted silica gel (gradient elution H₂O/CH₃CN) to give 81 mg of compound 59 (58%). LCMS (B): ES m/z=463 [M−H]⁻; m/z=465 [M+H]⁺; t_{R}=0.86 min.

### Compound 60: (S)-20-amino-14-oxo-2,5,8,11-tetraoxa-15-azahenicosan-21-oic acid hydrochloride

To compound 59 (150.6 mg, 324.2 μmol), under magnetic stirring, was added a 4M solution of HCl in 1,4-dioxane (1 mL). The reaction medium was stirred at RT overnight then concentrated in vacuo. EtOAc was added and the mixture was concentrated in vacuo to give 156 mg of compound 60 (quant.).

LCMS (B): ES m/z=508 [M−H]⁻; m/z=510 [M+H]⁺; t_{R}=1.23 min.

### Compound 61: (S)-20-((S)-2-((((9H-fluoren-9-yl)methoxy)carbonyl)amino)-3-methylbutanamido)-14-oxo-2,5,8,11-tetraoxa-15-azahenicosan-21-oic acid

To a solution of compound 60 (130 mg, 275 μmol) in water (0.5 mL) was added, under magnetic stirring, NaHCO₃ (55.6 mg, 661 μmol), followed by (S)-2,5-dioxopyrrolidin-1-yl 2-((((9H-fluoren-9-yl)methoxy)carbonyl)amino)-3-methylbutanoate (CAS number [130878-68-1], 144.4 mg, 330.8 μmol) in THF (1.5 mL). The reaction medium was stirred at RT overnight. Then, NaHCO₃ (30 mg), THF (500 μL), H₂O (200 μL) and (S)-2,5-dioxopyrrolidin-1-yl 2-((((9H-fluoren-9-yl)methoxy)carbonyl)-amino)-3-methylbutanoate (28 mg) were added to the reaction medium. After 3 h, the medium was concentrated in vacuo, then diluted with H₂O (20 mL) and −2-3 mL of aqueous saturated Na₂CO₃. The aqueous phase was extracted twice with EtOAc (20 mL). The combined organic phases were washed with brine (20 mL), dried over MgSO₄, filtered and concentrated in vacuo. The mixture was purified by flash chromatography on 15 g of silica gel (elution 85:1:0.5 v/v EtOAc/MeOH/H₂O). After concentration, the crude product was diluted with toluene and concentrated in vacuo to give 75.6 mg of compound 61 (40%).

RMN ¹H (400 MHz, δ in ppm, DMSO-d6): 0.85 (d, J=7.1 Hz, 3H); 0.87 (d, J=7.1 Hz, 3H); 1.21 to 1.40 (m, 4H); 1.50 to 1.73 (m, 2H); 2.01 (m, 1H); 2.27 (t, J=6.6 Hz, 2H); 2.98 (m, 2H); 3.23 (s, 3H); 3.41 (m, 2H); 3.43 to 3.52 (m, 10H); 3.57 (t, J=6.6 Hz, 2H); 3.88 (m, 1H); 4.01 (m, 1H); 4.19 to 4.34 (m, 3H); 7.31 (m, 2H); 7.38 to 7.46 (m, 3H); 7.70 to 7.91 (m, 6H); 12.51 (m, 1H). LCMS (A): ES m/z=684 [M−H]⁻; m/z=686 [M+H]⁺; t_{R}=1.2 min.

### Compound 62: N—((S)-5-((S)-2-amino-3-methylbutanamido)-6-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)amino)-6-oxohexyl)-2,5,8,11-tetraoxatetradecan-14-amide

To compound 61 (70 mg, 102.1 μmol), under magnetic stirring and Ar, was added a solution of (E)-(3S,10R,16S)-16-{(S)-1-[(2R,3R)-3-(4-aminomethyl-phenyl)-oxiranyl]-ethyl}-10-(3-chloro-4-methoxy-benzyl)-3-isobutyl-6,6-dimethyl-1,4-dioxa-8,11-diaza-cyclohexadec-13-ene-2,5,9,12-tetraone (the synthesis of which was described in WO2011001052, compound 77, 76.2 mg, 109.1 μmol) in THF (3 mL), followed by HOBt (17.7 mg, 131 μmol) and EDC (23 μL, 131 μmol). The reaction medium was stirred at RT overnight. Then, piperidine (32.8 μL, 327.4 μmol) was added to the medium, and stirring was maintained for 4 h 30. At this time, the medium was concentrated in vacuo, diluted with DMA and purified by flash chromatography on 15 g of C18-grafted silica gel (gradient elution H₂O/CH₃CN) to give 66 mg of compound 62(53%).

LCMS (B): ES m/z=572; m/z=1187 [M−H+HCO₂H]⁻; t_{R}=1.18 min.

### Example 21: (20S,23S)-20-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)-23-isopropyl-14,22,25-trioxo-2,5,8,11-tetraoxa-15,21,24-triazanonacosan-29-oic acid

To a solution of compound 62 (66 mg, 57.7 μmol) in DMF (4 mL), under magnetic stirring, was added glutaric anhydride (11.85 mg, 103.8 μmol). The reaction medium was stirred at RT for 2 h. At this time, the medium was concentrated in vacuo and purified by flash chromatography on 10 g of silica gel (gradient elution DCM/MeOH) to give 48.6 mg of example 21 (80%).

RMN ¹H (500 MHz, δ in ppm, DMSO-d6): 0.81 (m, 6H); 0.85 (d, J=7.5 Hz, 3H); 0.87 (d, J=7.5 Hz, 3H); 0.98 (s, 3H); 1.04 (d, J=7.1 Hz, 3H); 1.10 (s, 3H); 1.16 to 1.44 (m, 5H); 1.55 to 1.79 (m, 7H); 1.90 to 2.10 (m, 3H); 2.14 to 2.29 (m, 3H); 2.31 (t, J=6.7 Hz, 2H); 2.66 to 2.78 (m, 2H); 2.85 to 2.99 (m, 4H); 3.05 (m, 1H); 3.23 (s, 3 H); 3.32 (partially masked m, 1H); 3.42 (m, 2H); 3.44 to 3.51 (m, 10H); 3.57 (m, 2H); 3.81 (s, 3H); 3.88 (d, J=1.6 Hz, 1H); 4.04 to 4.25 (m, 4H); 4.35 (m, 1H); 4.94 (m, 1H); 5.10 (m, 1H); 5.80 (d, J=15.0 Hz, 1H); 6.45 (ddd, J=4.0, 11.2 and 15.0 Hz, 1H); 7.04 (d, J=8.6 Hz, 1H); 7.19 (dd, J=1.7 and 8.6 Hz, 1H); 7.22 (d, J=8.4 Hz, 2H); 7.28 (d, J=8.4 Hz, 2H); 7.30 (d, J=1.7 Hz, 1H); 7.32 (broad m, 1H); 7.75 to 9.42 (broad m, 5H); 12.06 (broad m, 1H). LCMS (A): ES m/z=629 [M+2H]²⁺; m/z=1255 [M−H]⁻; m/z=1257 [M+H]⁺; t_{R}=1.26 min.

### Example 22: (20S,23S)-2,5-dioxopyrrolidin-1-yl 20-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)-23-isopropyl-14,22,25-trioxo-2,5,8,11-tetraoxa-15,21,24-triazanonacosan-29-oate

Toluene (10 mL) was added to example 21 (46 mg, 36.6 μmol) and concentrated in vacuo, followed by dilution with THF (5 mL), DCM (2 mL) and DMF (200 μL). Then, DSC (11.5 mg, 43.9 μmol) was added, followed by DIEA (18.5 μL, 109.7 μmol). The reaction medium was stirred at RT for 4 h. At this time, MeTHF (7 mL) and H₂O (3 mL) were added, the aqueous phase was extracted twice with MeTHF (5 mL). The combined organic phases were washed with H₂O (3 mL), dried over MgSO₄, filtered and concentrated in vacuo. The crude medium was purified by flash chromatography on 4 g of silica gel (gradient elution DCM/iPrOH) to give 33.3 mg of example 22 (67%).

RMN ¹H (500 MHz, δ in ppm, DMSO-d6): 0.78 (d, J=6.6 Hz, 6H); 0.83 (d, J=7.1 Hz, 3H); 0.84 (d, J=7.1 Hz, 3H); 1.00 (s, 3H); 1.04 (d, J=6.9 Hz, 3H); 1.12 (s, 3H); 1.20 to 1.43 (m, 5H); 1.50 to 1.69 (m, 4H); 1.76 to 1.87 (m, 3H); 1.98 (m, 1H); 2.21 to 2.35 (m, 5H); 2.62 to 2.73 (m, 4H); 2.81 (s, 4H); 2.94 to 3.04 (m, 5H); 3.23 (s, 3H); 3.32 (partially masked m, 1H); 3.42 (m, 2H); 3.44 to 3.51 (m, 10H); 3.58 (t, J=6.4 Hz, 2H); 3.81 (s, 3H); 3.87 (d, J=1.1 Hz, 1H); 4.17 (dd, J=7.3 and 7.8 Hz, 1H); 4.21 to 4.31 (m, 4H); 4.90 (m, 1H); 5.10 (m, 1H); 5.79 (d, J=15.1 Hz, 1H); 6.47 (ddd, J=3.5, 11.5 and 15.1 Hz, 1H); 7.05 (d, J=8.5 Hz, 1H); 7.17 (dd, J=1.6 and 8.5 Hz, 1H); 7.20 to 7.25 (m, 5H); 7.28 (d, J=1.6 Hz, 1H); 7.79 (t, J=5.7 Hz, 1H); 7.91 (d, J=8.5 Hz, 1H); 7.95 (d, J=8.3 Hz, 1H); 8.37 (m, 2H). LCMS (C): ES m/z=677.5 [M+2H]²⁺; m/z=1354 [M+H]⁺; m/z=1399 [M−H+HCO₂H]⁻; t_{R}=4.41 min.

### Example 23: hu2H11 R35-74-Ex22

The general method described previously was used for the preparation of example 23. 44.64 mg of hu2H11_R35-74 were reacted with 209.2 μL of a 10 mM solution of example 22 in DMA (7 eq.) for 3 h 30. After purification on Sephadex G25 in buffer B pH 6.5+5% NMP and concentration on Amicon Ultra-15, 36.6 mg of example 23 were obtained as a colorless limpid solution at a concentration of 1.83 mg/mL with a DAR of 3.6 (HRMS), a monomeric purity of 98.8% and a global yield of 82%. Free-drug level was above the threshold of 1%: the ADC was concentrated on Amicon Ultra-15, purified on Sephadex G25 in buffer B pH 6.5+5% NMP and filtrated on 0.22 μm PVDF filter to provide 32.6 mg of example 23 as a colorless limpid solution at a concentration of 1.63 mg/mL with a DAR of 3.3 (HRMS), a monomeric purity of 98.3% and a global yield of 73%.

SEC-HRMS: m/z=149411 (naked mAb); m/z=150649 (D1); m/z=151889 (D2); m/z=153129 (D3); m/z=154369 (D4); m/z=155608 (D5); m/z=156850 (D6); m/z=158091 (D7); m/z=159331 (D8); m/z=160583 (D9).

### Synthesis of Examples 24 to 26: glutaryl-Val-PEG24Lys-C52 benzylic amine, NHS ester of glutaryl-Val-PEG24Lys-C52 benzylic amine and Corresponding ADC

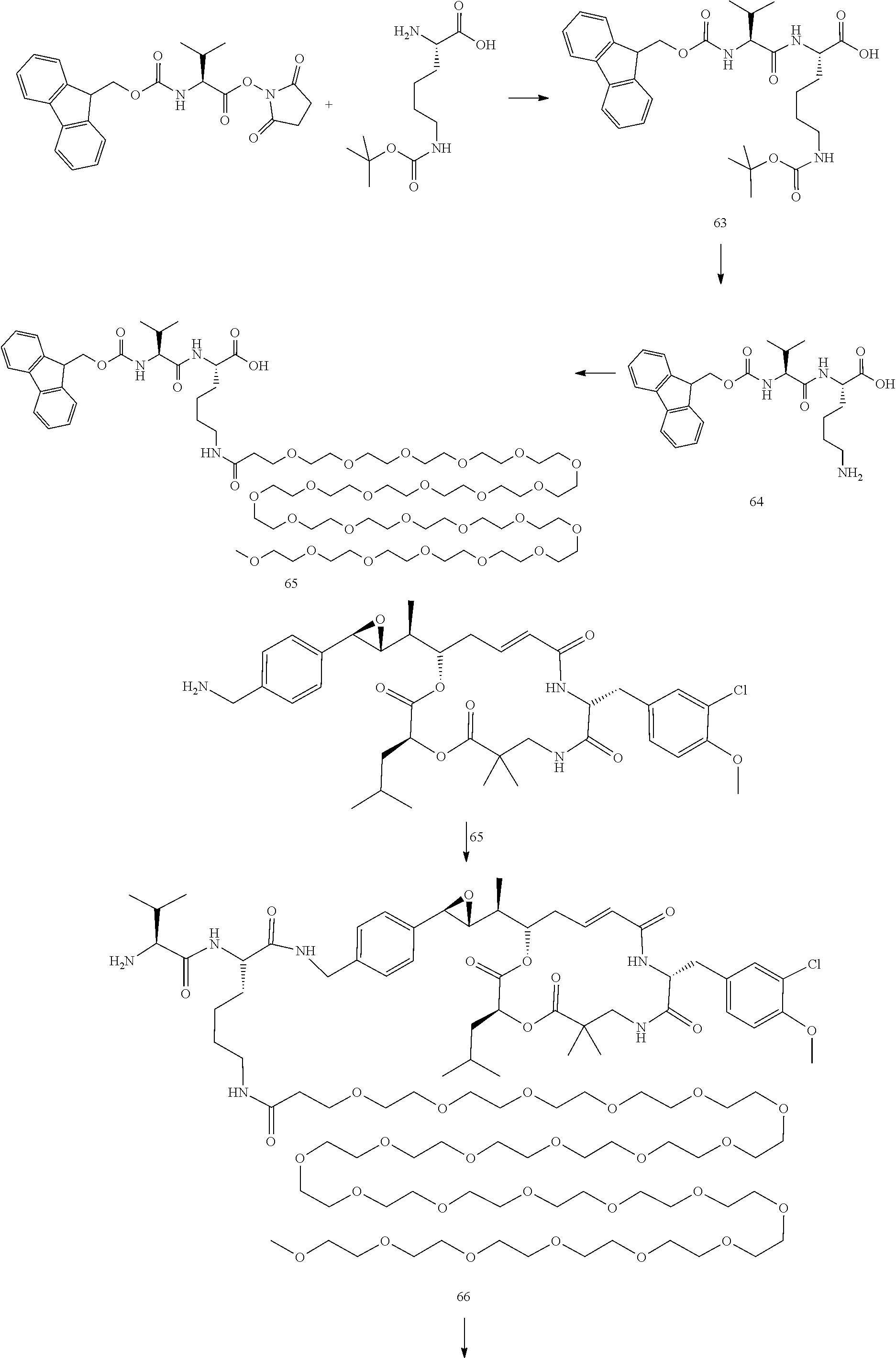

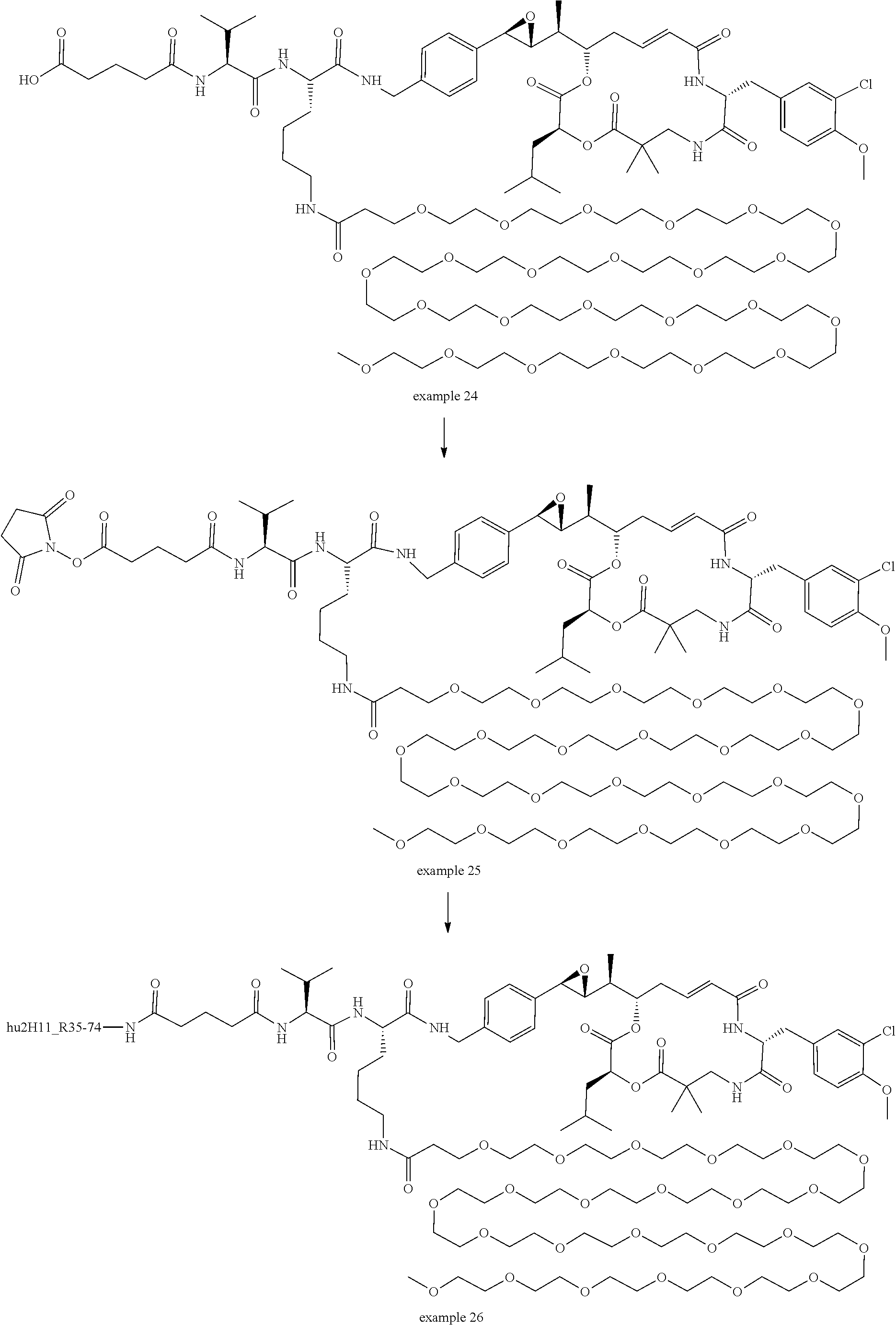

### Compound 63: (S)-2-((S)-2-((((9H-fluoren-9-yl)methoxy)carbonyl)amino)-3-methylbutanamido)-6-((tert-butoxycarbonyl)amino)hexanoic acid

To a solution of H-Lys(Boc)-OH (CAS number [2418-95-3], 310 mg, 1.26 mmol) and NaHCO₃ (105 mg, 1.25 mmol) in water (5 mL) and THF (5 mL) was added dropwise, under magnetic stirring, a solution of (S)-2,5-dioxopyrrolidin-1-yl 2-((((9H-fluoren-9-yl)methoxy)carbonyl)amino)-3-methylbutanoate (CAS number [68858-20-8], 310 mg, 1.15 mmol) in THF (15 mL). The reaction medium was stirred at RT for 3 h. At this time, the reaction medium was concentrated in vacuo, then diluted with water (200 mL), acidified with QS of aqueous 5N HCl, and extracted with DCM (2×200 mL). The combined organic phases were dried over MgSO₄, filtered and concentrated in vacuo to afford 510 mg of compound 63 (53%).

RMN ¹H (400 MHz, δ in ppm, DMSO-d6): 0.85 (d, J=6.8 Hz, 3H); 0.87 (d, J=6.8 Hz, 3H); 1.20 to 1.39 (m, 4H); 1.34 (s, 9H); 1.48 to 1.73 (m, 2H); 1.99 (m, 1H); 2.86 (m, 2H); 3.89 (dd, J=7.7 and 8.4 Hz, 1H); 4.08 (m, 1H); 4.17 to 4.32 (m, 3H); 6.72 (broad m, 1H); 7.32 (m, 2H); 7.38 to 7.46 (m, 3H); 7.74 (m, 2H); 7.89 (d, J=7.6 Hz, 2H); 8.00 (broad m, 1H); 12.54 (broad s, 1H). LCMS (A): ES m/z=344; m/z=468; m/z=566 [M−H]⁻; m/z=568 [M+H]⁺; t_{R}=1.41 min.

### Compound 64: (S)-2-((S)-2-((((9H-fluoren-9-yl)methoxy)carbonyl)amino)-3-methylbutanamido)-6-aminohexanoic acid hydrochloride

To compound 63 (340 mg, 599 μmol) was added a solution of 4M HCl in 1,4-dioxane, the reaction medium was stirred at RT for 1 h. At this time, the reaction medium was concentrated in vacuo, followed by addition of Et₂O and filtration. The white solid obtained was purified by flash chromatography on 15 g of silica gel (elution 12:3:0.5 v/v/v DCM/MeOH/NH₄OH) to give 190 mg of compound 64 (63%).

RMN ¹H (400 MHz, δ in ppm, DMSO-d6): 0.83 (d, J=6.6 Hz, 3H); 0.85 (d, J=6.6 Hz, 3H); 1.21 to 1.70 (m, 6H); 2.04 (m, 1H); 2.71 (t, J=7.1 Hz, 2H); 3.84 (dd, J=6.7 and 8.7 Hz, 1H); 3.91 (m, 1H); 4.18 to 4.35 (m, 3H); 7.22 (broad m, 3H); 7.33 (m, 2H); 7.42 (t, J=7.6 Hz, 2H); 7.54 (d, J=9.0 Hz, 1H); 7.69 (broad m, 1H); 7.74 (m, 2H); 7.89 (d, J=7.6 Hz, 2H). LCMS (A): ES m/z=244; m/z=466 [M−H]⁻; m/z=468 [M+H]⁺; t_{R}=0.81 min.

### Compound 65: (S)-80-((S)-2-((((9H-fluoren-9-yl)methoxy)carbonyl)amino)-3-methylbutanamido)-74-oxo-2,5,8,11,14,17,20,23,26,29,32,35,38,41,44,47,50,53,56, 59,62,65,68,71-tetracosaoxa-75-azahenoctacontan-81-oic acid

To a solution of compound 64 (130 mg, 258.0 μmol) in THF (20 mL) were added a solution of sodium bicarbonate (50 mg, 589.2 μmol) in H₂O (8 mL) and MEO-DPEG(24)—NHS (CAS number [756525-94-7], 400 mg, 329.4 μmol). The reaction medium was stirred for 20 h at RT, partly concentrated in vacuo then diluted with H₂O (10 mL), acidified to pH 3 with IR-120 (H) Amberlite resin (CAS number [78922-04-0]), filtered, concentrated in vacuo and purified by three consecutive flash chromatographies on 40 g, 25 g and 12 g of silica gel (gradient elution DCM/MeOH/H₂O) to give 214 mg of compound 65 as a white lacquer (53%).

RMN ¹H (400 MHz, δ in ppm, DMSO-d6): 0.86 (d, J=7.0 Hz, 3H); 0.89 (d, J=7.0 Hz, 3H); 1.22 to 1.40 (m, 4H); 1.58 (m, 1H); 1.67 (m, 1H); 1.99 (m, 1H); 2.28 (t, J=6.6 Hz, 2H); 2.99 (m, 2H); 3.23 (s, 3H); 3.40 to 3.70 (m, 92H); 3.56 (t, J=6.6 Hz, 2H); 3.90 (m, 1H); 4.08 (broad m, 1H); 4.19 to 4.31 (m, 3H); 7.31 (broad t, J=7.8 Hz, 2H); 7.41 (broad t, J=7.8 Hz, 3H); 7.73 (t, J=7.8 Hz, 2H); 7.76 (m, 1H); 7.89 (d, J=7.8 Hz, 2H); 8.00 (m, 1H); 12.53 (m, 1H). LCMS (D): ES m/z=794 [M+H+Na]²⁺; m/z=1567 [M+H]⁺; t_{R}=2.9 min.

### Compound 66: N—((S)-5-((S)-2-amino-3-methylbutanamido)-6-((4-((2R,3R)-3-((S)-1-((3S,10R, 16S,E)-10-(3-chloro-4-methoxybenzyl)-3-isobutyl-6,6-dimethyl-2,5,9,12-tetraoxo-1,4dioxa-8,11-diazacyclohexadec-13-en-16-yl)ethyl)oxiran-2-yl)benzyl)-amino)-6-oxohexyl)-2,5,8,11,14,17,20,23,26,29,32,35,38,41,44,47,50,53,56,59,62, 65,68,71-tetracosaoxatetraheptacontan-74-amide

To a solution of (E)-(3S,10R,16S)-16-{(S)-1-[(2R,3R)-3-(4-aminomethyl-phenyl)-oxiranyl]-ethyl}-10-(3-chloro-4-methoxy-benzyl)-3-isobutyl-6,6-dimethyl-1,4-dioxa-8,11-diaza-cyclohexadec-13-ene-2,5,9,12-tetraone (the synthesis of which was described in WO2011001052, compound 77.71 mg, 101.7 μmol) in DMF (5 mL) were added a solution of compound 65 (214 mg, 136.6 μmol) in DMF (5 mL), HOBt (22 mg, 156.3 μmol) and EDC (27 μL, 152.5 μmol). The reaction medium was stirred for 3 h at RT before adding piperidine (91 μL, 915.3 μmol). Stirring was carried on for 1 h 30 at RT then the reaction medium was concentrated in vacuo and purified by two consecutive flash chromatographies on 25 g and 12 g of silica gel (gradient elution DCM/MeOH) to give 58 mg of compound 66 as a colorless lacquer (28%).

RMN ¹H (500 MHz, δ in ppm, DMSO-d6): 0.76 (d, J=7.0 Hz, 3H); 0.78 (d, J=6.8 Hz, 6H); 0.88 (d, J=7.0 Hz, 3H); 0.99 (s, 3H); 1.04 (d, J=7.1 Hz, 3H); 1.12 (s, 3H); 1.25 to 1.41 (m, 5H); 1.51 to 1.70 (m, 4H); 1.80 (m, 1H); 1.93 (m, 1H); 2.25 (m, 1H); 2.28 (t, J=6.7 Hz, 2H); 2.55 (broad m, 2H); 2.68 (m, 2H); 2.99 (m, 5H); 3.23 (s, 3H); 3.32 (masked m, 1H); 3.40 to 3.55 (m, 92H); 3.58 (t, J=6.7 Hz, 2H); 3.80 (s, 3H); 3.88 (d, J=2.3 Hz, 1H); 4.20 to 4.35 (m, 4H); 4.91 (dd, J=3.8 and 9.8 Hz, 1H); 5.10 (m, 1H); 5.80 (dd, J=2.0 and 15.8 Hz, 1H); 6.46 (ddd, J=4.7, 10.5 and 15.8 Hz, 1H); 7.05 (d, J=8.6 Hz, 1H); 7.17 (dd, J=2.4 et 8.6 Hz, 1H); 7.22 (m, 5H); 7.29 (d, J=2.4 Hz, 1H); 7.79 (t, J=6.7 Hz, 1H); 8.00 (d, J=8.8 Hz, 1H); 8.36 (d, J=8.0 Hz, 1H); 8.47 (t, J=6.2 Hz, 1H). LCMS (D): ES m/z=1013 [M+2H]²⁺; m/z=2025 [M+H]⁺; t_{R}=2.64/2.62 min (85/15 isomer mixture).

### Example 24: (80S,83S)-80-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)-83-isopropyl-74,82,85-trioxo-2,5,8,11,14,17,20,23,26,29,32,35,38,41,44,47,50,53,56,59,62,65, 68,71-tetracosaoxa-75,81,84-triazanonaoctacontan-89-oic acid

To a solution of compound 66 (58 mg, 28.7 μmol) in DCM (7 mL) was added glutaric anhydride (5.85 mg, 48.7 μmol). The reaction medium was stirred for 2 h at RT, concentrated in vacuo and purified by flash chromatography on 4 g of silica gel (gradient elution DCM/MeOH/H₂O) to give 35 mg of example 24 as a white lacquer (57%).

RMN ¹H (500 MHz, δ in ppm, DMSO-d6): 0.78 (d, J=6.8 Hz, 6H); 0.82 (d, J=7.0 Hz, 3H); 0.84 (d, J=7.0 Hz, 3H); 1.00 (s, 3H); 1.04 (d, J=7.1 Hz, 3H); 1.11 (s, 3H); 1.18 to 1.41 (m, 5H); 1.50 to 1.75 (m, 6H); 1.80 (m, 1H); 1.98 (m, 1H); 2.15 to 2.27 (m, 5H); 2.30 (t, J=6.7 Hz, 2H); 2.69 (m, 2H); 2.98 (m, 5H); 3.23 (s, 3H); 3.32 (masked m, 1H); 3.40 to 3.65 (m, 94H); 3.80 (s, 3H); 3.87 (d, J=2.3 Hz, 1H); 4.14 (m, 1H); 4.20 to 4.33 (m, 4H); 4.90 (dd, J=3.8 and 9.8 Hz, 1H); 5.10 (m, 1H); 5.79 (d, J=15.8 Hz, 1H); 6.47 (ddd, J=4.7, 10.5 and 15.8 Hz, 1H); 7.05 (d, J=8.6 Hz, 1H); 7.16 (dd, J=2.4 and 8.6 Hz, 1H); 7.24 (m, 5H); 7.29 (d, J=2.4 Hz, 1H); 7.80 (t, J=6.0 Hz, 1H); 7.86 (d, J=8.8 Hz, 1H); 7.92 (d, J=8.0 Hz, 1H); 8.37 (m, 2H); 12.10 (broad m, 1H). LCMS (D): ES m/z=1069.5 [M+2H]²⁺; m/z=2138 [M+H]⁺; t_{R}=2.96/2.93 min (85/15 isomer mixture).

### Example 25: (80S,83S)-2,5-dioxopyrrolidin-1-yl 80-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclo-hexadec-13-en-16-yl)ethyl)oxiran-2-yl)benzyl)carbamoyl)-83-isopropyl-74,82,85-trioxo-2,5,8,11,14,17,20,23,26,29,32,35, 38,41,44,47,50,53,56,59,62,65,68,71-tetracosaoxa-75,81,84-triazanona-octacontan-89-oate

To a solution of example 24 (35 mg, 16.4 μmol) in THF (3 mL) were added DSC (4.28 mg, 16.4 μmol) and DIEA (2.7 μL, 16.4 μmol). The reaction medium was stirred for 20 h at RT, concentrated in vacuo and purified by flash chromatography on 4 g of silica gel (gradient elution DCM/iPrOH) to give 12.0 mg of example 25 as a white lacquer (33%).

RMN ¹H (500 MHz, δ in ppm, DMSO-d6): 0.78 (d, J=6.8 Hz, 6H); 0.82 (d, J=7.0 Hz, 3H); 0.84 (d, J=7.0 Hz, 3H); 1.00 (s, 3H); 1.04 (d, J=7.1 Hz, 3H); 1.12 (s, 3H); 1.15 to 1.40 (m, 5H); 1.50 to 1.71 (m, 4H); 1.75 to 1.86 (m, 3H); 1.97 (m, 1H); 2.18 to 2.32 (m, 5H); 2.67 (m, 4H); 2.80 (s, 4H); 2.98 (m, 5H); 3.22 (s, 3H); 3.32 (masked m, 1H); 3.40 to 3.77 (m, 94H); 3.80 (s, 3H); 3.88 (d, J=2.3 Hz, 1H); 4.16 (m, 1H); 4.20 to 4.32 (m, 4H); 4.90 (dd, J=3.8 and 9.8 Hz, 1H); 5.10 (m, 1H); 5.78 (dd, J=2.0 and 15.8 Hz, 1H); 6.47 (ddd, J=4.7, 10.5 and 15.8 Hz, 1H); 7.05 (d, J=8.6 Hz, 1H); 7.16 (dd, J=2.4 and 8.6 Hz, 1H); 7.24 (m, 5H); 7.29 (d, J=2.4 Hz, 1H); 7.79 (t, J=6.0 Hz, 1H); 7.90 (d, J=8.8 Hz, 1H); 7.94 (d, J=8.0 Hz, 1H); 8.36 (m, 2H). LCMS (D): ES m/z=746; m/z=2235 [M+H]⁺; t_{R}=3.08/3.06 min (89/11 isomer mixture).

### Example 26: hu2H11_R35-74-Ex25

The general method described previously was used for the preparation of example 26. 55.8 mg of hu2H11_R35-74 were reacted with 261.6 μL of a 10 mM solution of example 25 in DMA (7 eq.) for 3 h 15 then were added 131 μL of a 10 mM solution of example 25 in DMA (3.5 eq.) for 5 h 30. After storage overnight at 4° C., purification on Superdex 200 μg in buffer B pH 6.5+20% NMP, concentration on Amicon Ultra-15, buffer exchange on Sephadex G25 in buffer B pH 6.5+5% NMP, concentration on Amicon Ultra-15 and filtration on 0.22 μm PVDF filter, 33.35 mg of example 26 were obtained as a colorless limpid solution at a concentration of 2.9 mg/mL with a DAR of 3.9 (HRMS), a monomeric purity of 100% and a global yield of 60%.

SEC-HRMS: m/z=151487 (D1); m/z=153615 (D2); m/z=155740 (D3); m/z=157863 (D4); m/z=159981 (D5); m/z=162112 (D6); m/z=164240 (D7); m/z=166356 (D8).

### Synthesis of Examples 27 to 29: glutaryl-Val-PEG24Lys-aza-crypto benzylic amine, NHS ester of glutaryl-Val-PEG24Lys-aza-crypto benzylic amine and Corresponding ADC

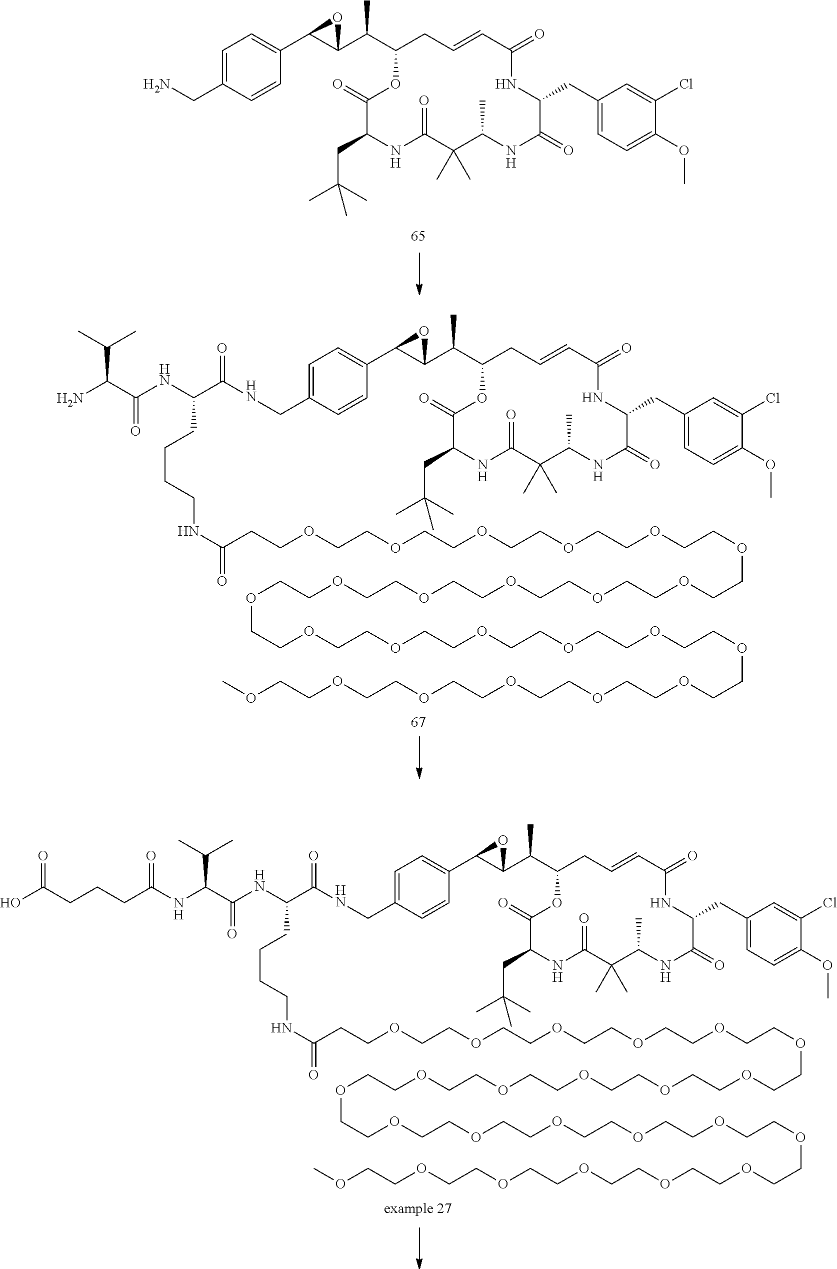

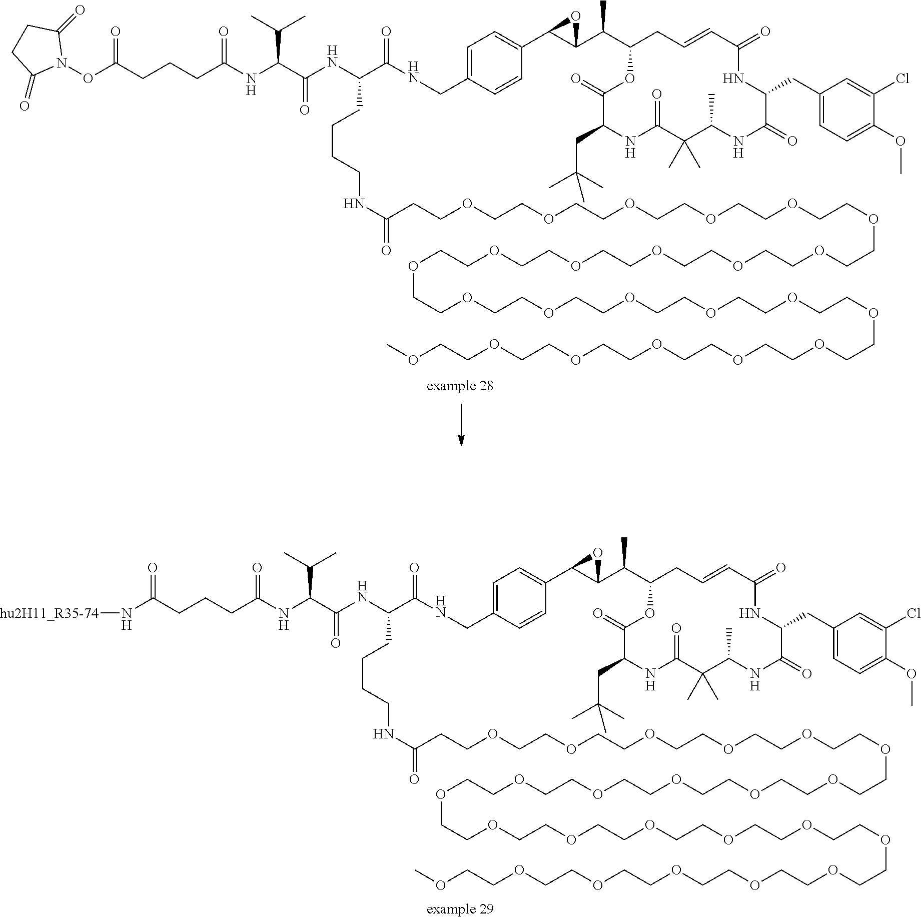

### Compound 67: N—((S)-5-((S)-2-amino-3-methylbutanamido)-6-((4-((2R,3R)-3-((S)-1-((3S,7S, 10R,16S,E)-10-(3-chloro-4-methoxybenzyl)-6,6,7-trimethyl-3-neopentyl-2,5,9,12-tetraoxo-1-oxa-4,8,11-triazacyclohexadec-13-en-16-yl)ethyl)oxiran-2-yl)benzyl)amino)-6-oxohexyl)-2,5,8,11,14,17,20,23,26,29,32,35,38,41,44,47,50,53, 56,59,62,65,68,71-tetracosaoxatetraheptacontan-74-amide

To a solution of (3S,7S,10R,16S,E)-16-((S)-1-((2R,3R)-3-(4-(aminomethyl)phenyl)-oxiran-2-yl)ethyl)-10-(3-chloro-4-methoxybenzyl)-6,6,7-trimethyl-3-neopentyl-1-oxa-4,8,11-triazacyclo-hexadec-13-ene-2,5,9,12-tetraone (that can be synthesized as described in PCT/EP2016/076603 starting from methyl (3S)-3-amino-2,2-dimethylbutanoate, [MFCD09256689], 75 mg, 103.4 μmol) in DMF (5 mL) were added a solution of compound 65 (211 mg, 134.7 μmol) in DMF (5 mL), HOBt (21 mg, 155.4 μmol) and EDC (20 μL 123.0 μmol). The reaction medium was stirred for 4 h at RT before adding piperidine (95 μL, 961.9 μmol); stirring was carried on for 2 h at RT then the reaction medium was concentrated in vacuo and purified by flash chromatography on 25 g of silica gel (gradient elution DCM/MeOH) to give 132 mg of compound 67 as a colorless oil (62%).

RMN ¹H (500 MHz, δ in ppm, DMSO-d6): 0.76 (d, J=7.0 Hz, 3H); 0.87 (m, 15H); 1.00 (s, 3H); 1.03 (d, J=7.1 Hz, 3H); 1.15 to 1.40 (m, 5H); 1.18 (s, 3H); 1.46 to 2.00 (m, 8H); 2.25 (m, 1H); 2.28 (t, J=6.7 Hz, 2H); 2.60 (m, 1H); 2.69 (dd, J=11.2 and 14.5 Hz, 1H); 2.91 (dd, J=2.3 and 7.5 Hz, 1H); 2.92 to 3.04 (m, 4H); 3.23 (s, 3H); 3.34 to 3.77 (m, 95H); 3.80 (s, 3H); 3.88 (d, J=2.3 Hz, 1H); 4.08 (m, 2H); 4.20 to 4.34 (m, 3H); 5.04 (m, 1H); 5.78 (dd, J=2.0 and 15.8 Hz, 1H); 6.44 (ddd, J=4.7, 10.5 and 15.8 Hz, 1H); 7.02 (d, J=8.6 Hz, 1H); 7.17 to 7.25 (m, 5H); 7.32 (d, J=2.4 Hz, 1H); 7.78 (t, J=6.0 Hz, 1H); 7.85 (d, J=8.0 Hz, 1H); 7.92 (d, J=7.0 Hz, 1H); 8.00 (d, J=8.8 Hz, 1H); 8.40 (t, J=7.5 Hz, 1H); 8.46 (t, J=6.0 Hz, 1H). LCMS (D): ES m/z=2051 [M+H]⁺; t_{R}=2.57/2.55 min (isomer mixture 95/5).

### Example 27: (80S,83S)-80-((4-((2R,3R)-3-((S)-1-((3S,7S,10R,16S,E)-10-(3-chloro-4-methoxybenzyl)-6,6,7-trimethyl-3-neopentyl-2,5,9,12-tetraoxo-1-oxa-4,8,11-triazacyclohexadec-13-en-16-yl)ethyl)oxiran-2-yl)benzyl)carbamoyl)-83-isopropyl-74,82,85-trioxo-2,5,8,11,14,17,20,23,26,29,32,35,38,41,44,47,50,53,56,59,62,65, 68,71-tetracosaoxa-75,81,84-triazanonaoctacontan-89-oic acid

To a solution of compound 67 (132 mg, 64.3 μmol) in DCM (10 mL) was added glutaric anhydride (9 mg, 78.9 μmol). The reaction medium was stirred for 2 h at RT, concentrated in vacuo and purified by two consecutive flash chromatographies on 4 g and 12 g of silica gel (gradient elution DCM/MeOH/H₂O) to give 71 mg of example 27 as a white lacquer (51%).

RMN ¹H (500 MHz, δ in ppm, DMSO-d6): 0.80 to 0.90 (m, 18H); 1.00 (s, 3H); 1.03 (d, J=7.1 Hz, 3H); 1.15 to 1.42 (m, 5H); 1.18 (s, 3H); 1.51 to 1.74 (m, 4H); 1.80 (m, 1H); 1.98 (m, 2H); 2.15 to 2.32 (m, 7H); 2.60 (m, 1H); 2.70 (dd, J=11.2 and 14.5 Hz, 1H); 2.91 (dd, J=2.3 and 7.5 Hz, 1H); 2.95 (dd, J=3.4 and 14.5 Hz, 1H); 3.00 (m, 2H); 3.24 (s, 3H); 3.40 to 3.67 (m, 95H); 3.80 (s, 3H); 3.90 (d, J=2.3 Hz, 1H); 4.00 to 4.34 (m, 6H); 5.04 (m, 1H); 5.80 (dd, J=2.0 and 15.8 Hz, 1H); 6.44 (ddd, J=4.7, 10.5 and 15.8 Hz, 1H); 7.02 (d, J=8.6 Hz, 1H); 7.17 to 7.25 (m, 5H); 7.32 (d, J=2.4 Hz, 1H); 7.80 (t, J=6.0 Hz, 1H); 7.86 (d, J=8.6 Hz, 2H); 7.92 (t, J=6.5 Hz, 2H); 8.37 (t, J=6.0 Hz, 1H); 8.41 (t, J=7.5 Hz, 1H); 12.00 (m, 1H). LCMS (D): ES m/z=722; m/z=2165 [M+H]⁺; t_{R}=2.95/2.92 min (isomer mixture 95/5).

### Example 28: 2,5-dioxopyrrolidin-1-yl (80S,83S)-80-((4-((2R,3R)-3-((S)-1-((3S,7S,10R,16S,E)-10-(3-chloro-4-methoxybenzyl)-6,6,7-trimethyl-3-neopentyl-2,5,9,12-tetraoxo-1-oxa-4,8,11-triaza-cyclohexadec-13-en-16-yl)ethyl)oxiran-2-yl)benzyl)carbamoyl)-83-isopropyl-74,82,85-trioxo-2,5,8,11,14,17,20,23,26,29,32,35, 38,41,44,47,50,53,56,59,62,65,68,71-tetracosaoxa-75,81,84-triazanonaoctacontan-89-oate

To a solution of example 27 (33 mg, 15.2 μmol) in THF (3 mL) were added DSC (4 mg, 15.6 μmol) and DIEA (2.55 μL, 15.4 μmol). The reaction medium was stirred for 20 h at RT then were added DSC (1 mg, 3.9 μmol) and DIEA (1 μL, 6.0 μmol). The reaction medium was stirred for 2 h at RT, concentrated in vacuo and purified by flash chromatography on 4 g of silica gel (gradient elution DCM/iPrOH/MeCN) to give 11 mg of example 28 as a white lacquer (32%).

RMN ¹H (500 MHz, δ in ppm, DMSO-d6): 0.80 to 0.90 (m, 18H); 1.00 (s, 3H); 1.03 (d, J=7.1 Hz, 3H); 1.12 to 1.42 (m, 5H); 1.19 (s, 3H); 1.55 (m, 1H); 1.65 (m, 1H); 1.82 (m, 3H); 1.96 (m, 2H); 2.28 (m, 3H); 2.67 (m, 4H); 2.80 (s, 4H); 2.91 (dd, J=2.3 and Hz, 1H); 2.95 (dd, J=3.4 and 14.5 Hz, 1H); 3.00 (m, 2H); 3.23 (s, 3H); 3.35 to 3.67 (m, 95H); 3.80 (s, 3H); 3.90 (d, J=2.3 Hz, 1H); 4.03 to 4.32 (m, 6H); 5.03 (m, 1H); 5.90 (dd, J=2.0 and 15.8 Hz, 1H); 6.44 (ddd, J=4.7, 10.5 and 15.8 Hz, 1H); 7.03 (d, J=8.6 Hz, 1H); 7.17 to 7.25 (m, 5H); 7.32 (d, J=2.4 Hz, 1H); 7.79 (t, J=6.0 Hz, 1H); 7.85 (d, J=8.7 Hz, 1H); 7.90 (d, J=8.7 Hz, 1H); 7.93 (m, 2H); 8.35 (t, J=6.5 Hz, 1H); 8.40 (d, J=7.1 Hz, 1H).

### Example 29: hu2H11_R35-74-Ex28

The general method described previously was used for the preparation of example 29. 44.64 mg of hu2H11_R35-74 were reacted with 210 μL of a 10 mM solution of example 28 in DMA (7 eq.) for 2 h then were successively added 210 μL of a 10 mM solution of example 28 in DMA (7 eq.) for 2 h 30 and 90 μL of a 10 mM solution of example 28 in DMA (3 eq.) for 2 h. After storage overnight at 4° C., purification on Superdex 200 μg in buffer B pH 6.5+20% NMP, concentration on Amicon Ultra-15, buffer exchange on Sephadex G25 in buffer B pH 6.5+5% NMP and filtration on 0.22 μm PVDF filter, 30.1 mg of example 29 were obtained as a colorless limpid solution at a concentration of 2.51 mg/mL with a DAR of 4 (HRMS), a monomeric purity of 98.4% and a global yield of 67%.

SEC-HRMS: m/z=151502 (D1); m/z=153649 (D2); m/z=155798 (D3); m/z=157939 (D4); m/z=160100 (D5); m/z=162262 (D6).

### Synthesis of Examples 30 to 32: glutaryl-Val-GlucuronicLys-C52 benzylic amine, NHS ester of glutaryl-Val-GlucuronicLys-C52 benzylic amine and Corresponding ADC

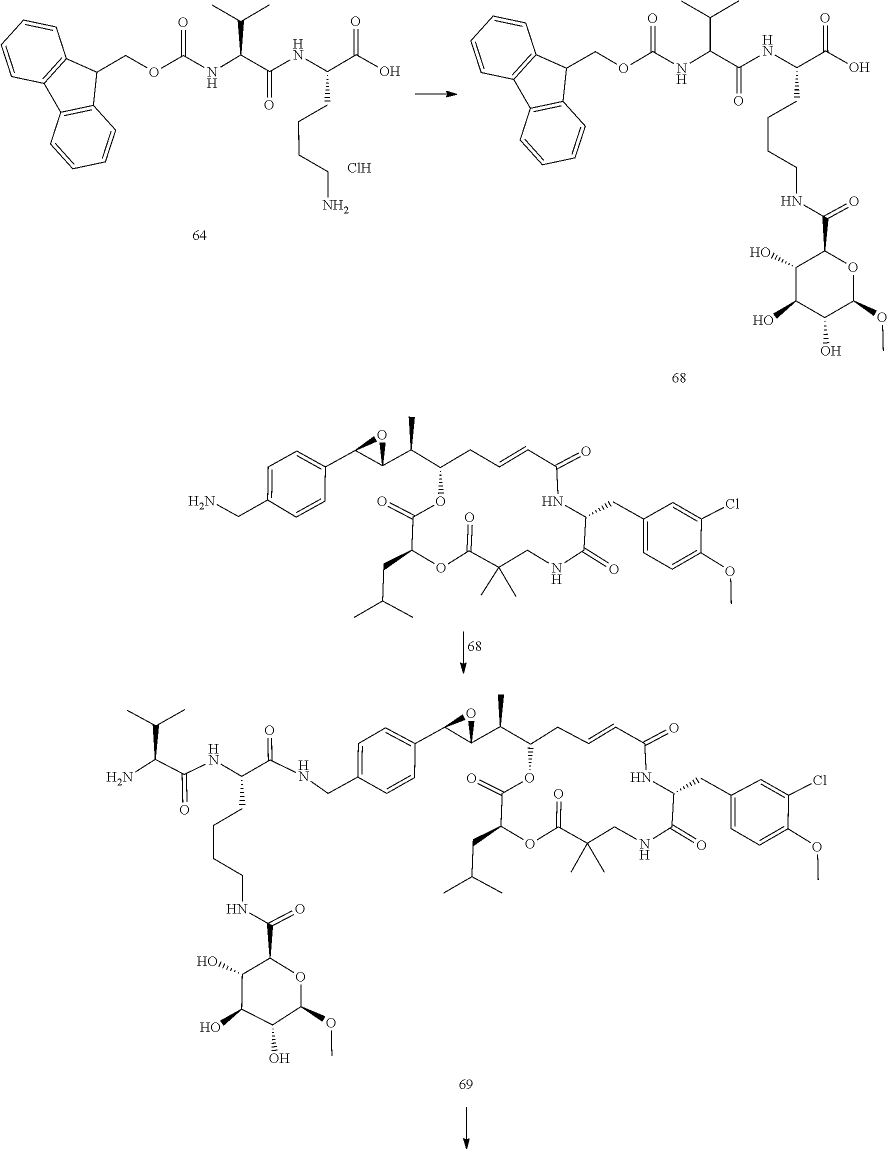

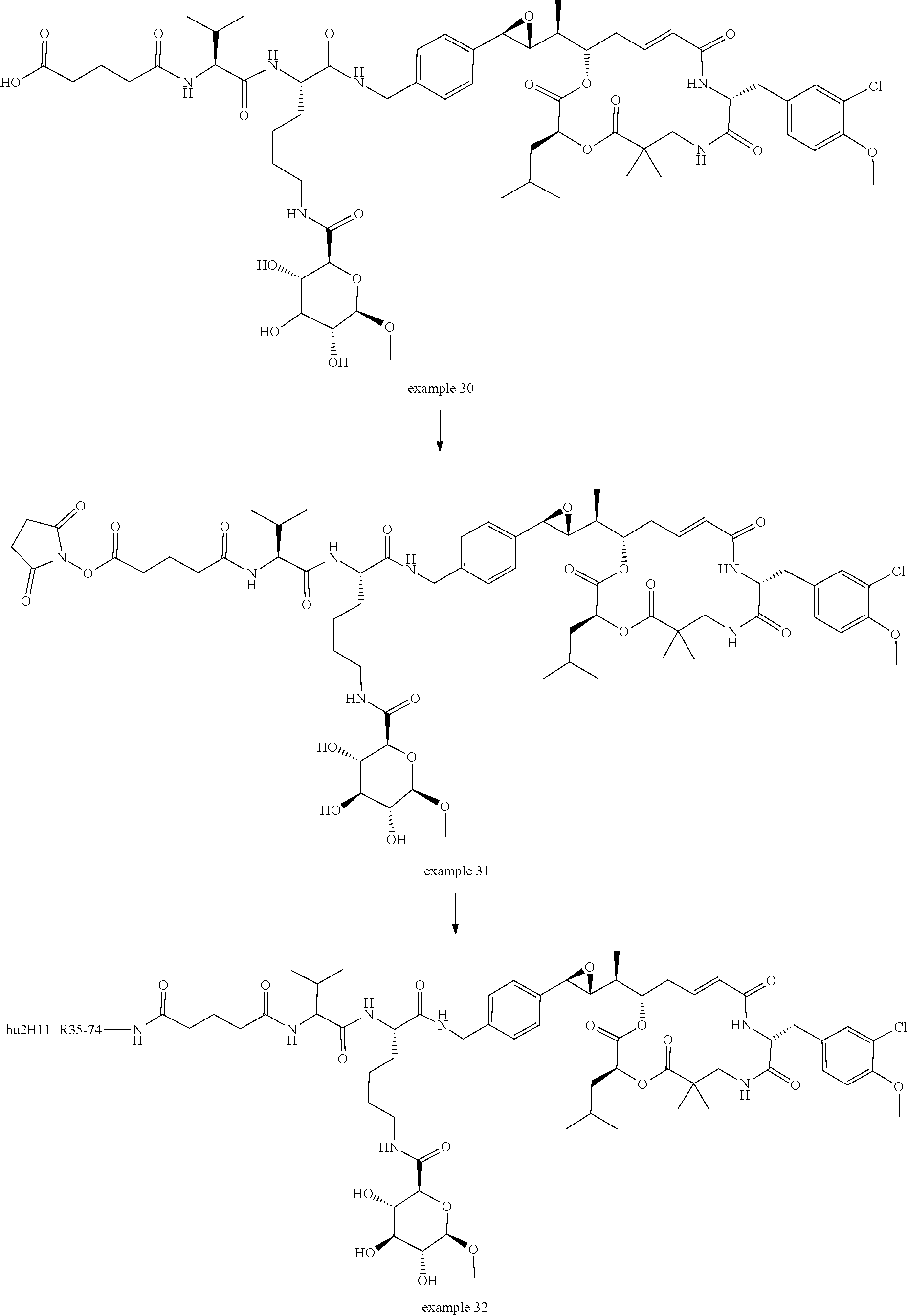

### Compound 68: (S)-2-((S)-2-((((9H-fluoren-9-yl)methoxy)carbonyl)amino)-3-methylbutanamido)-6-((2S,3S,4S,5R,6R)-3,4,5-trihydroxy-6-methoxytetrahydro-2H-pyran-2-carboxamido)hexanoic acid

In a round bottom flask, under magnetic stirring, 1-O-methyl-β-D-glucuronic acid, sodium salt (CAS number [58189-74-5], 100 mg, 434 μmol) was introduced, followed by water (5 mL) and QS of Amberlite (CAS number [9037-24-5]) to reach pH 2, the medium was filtered.

In a round bottom flask, under magnetic stirring, NaHCO₃ (43 mg, 511 μmol) in water (5 mL) was added to a solution compound 64 (50 mg, 99 μmol) in THF (4 mL); the reaction medium was stirred at RT for 5 min. Then, the glucuronic acid solution described above was added to the reaction medium, followed by 4-(4,6-dimethoxy-1,3,5-triazin-2-yl)-4-methylmorpholinium chloride N-hydrate (CAS number [3945-69-5], 67 mg, 242 μmol). The reaction medium was stirred at RT overnight, then concentrated in vacuo. The crude medium was purified by flash chromatography on 5 g of silica gel (gradient elution DCM/MeOH/H₂O) to give 23 mg of compound 68 (35%).

RMN ¹H (400 MHz, δ in ppm, DMSO-d6): 0.84 (d, J=6.7 Hz, 3H); 0.86 (d, J=6.7 Hz, 3H); 1.13 to 1.42 (m, 4H); 1.52 to 1.72 (m, 2H); 2.05 (m, 1H); 2.96 to 3.08 (m, 2H); 3.14 (m, 1H); 3.30 (partially masked m, 1H); 3.38 (s, 3H); 3.53 (m, 1H); 3.78 to 3.97 (m, 2H); 4.09 (dd, J=2.3 and 7.8 Hz, 1H); 4.19 to 4.34 (m, 3H); 5.01 to 5.08 (broad m, 1H); 5.13 (broad m, 1H); 5.27 (m, 1H); 7.33 (m, 2H); 7.42 (t, J=7.6 Hz, 2H); 7.52 to 7.62 (broad m, 2H); 7.75 (m, 2H); 7.89 (d, J=7.6 Hz, 2H); 7.93 (broad m, 1H); 12.00 (m, 1H). LCMS (A): ES m/z=656 [M−H]⁻; m/z=658 [M+H]⁺; t_{R}=1.05 min.

### Compound 69: (2S,3S,4S,5R,6R)—N—((S)-5-((S)-2-amino-3-methylbutanamido)-6-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)-oxiran-2-yl)benzyl)amino)-6-oxohexyl)-3,4,5-trihydroxy-6-methoxytetrahydro-2H-pyran-2-carboxamide

To a solution of (E)-(3S,10R,16S)-16-{(S)-1-[(2R,3R)-3-(4-aminomethyl-phenyl)-oxiranyl]-ethyl}-10-(3-chloro-4-methoxy-benzyl)-3-isobutyl-6,6-dimethyl-1,4-dioxa-8,11-diaza-cyclohexadec-13-ene-2,5,9,12-tetraone (the synthesis of which was described in WO2011001052, compound 77, 90 mg, 129 μmol) was added, under magnetic stirring, a solution of compound 68 (135 mg, 205 μmol) in DMF (5 mL), followed by HOBt (30 mg, 222 μmol) and EDC (36 μL, 203 μmol). The reaction medium was stirred at RT for 1 h. At this time, DMA (1 mL) was added, and stirring was maintained overnight. Then, EDC (20 μL) was added and the reaction medium was stirred for 2 more hours. Piperidine (12.9 μL, 129 μmol) was then added to the medium. After 1 h30 of stirring, the reaction medium was concentrated in vacuo and purified by 3 consecutive flash chromatographies on silica gel (isocratic elution 40:5:0.5 v/v/v DCM/MeOH/H₂O and 12:3:0.5 v/v/v DCM/MeOH/NH₄OH) to give 47 mg of compound 69 (33%).

RMN ¹H (500 MHz, δ in ppm, DMSO-d6): 0.77 to 0.81 (m, 9H); 0.88 (d, J=6.6 Hz, 3H); 1.00 (s, 3H); 1.04 (d, J=6.6 Hz, 3H); 1.12 (s, 3H); 1.20 to 1.71 (m, 9H); 1.80 (m, 1H); 1.93 (m, 1H); 2.28 (m, 1H); 2.62 to 2.73 (m, 2H); 2.93 to 3.14 (m, 9H); 3.28 to 3.36 (partially masked m, 2H); 3.39 (s, 3H); 3.53 (d, J=9.5 Hz, 1H); 3.81 (s, 3H); 3.87 (d, J=1.7 Hz, 1H); 4.09 (m, 1H); 4.19 to 4.36 (m, 4H); 4.91 (m, 1H); 5.00 to 5.15 (m, 4H); 5.79 (d, J=15.9 Hz, 1H); 6.48 (m, 1H); 7.05 (d, J=8.6 Hz, 1H); 7.17 (dd, J=2.1 and 8.6 Hz, 1H); 7.20 to 7.25 (m, 5H); 7.28 (d, J=2.1 Hz, 1H); 7.92 (broad t, J=5.9 Hz, 1H); 8.02 (broad m, 1H); 8.35 (d, J=7.8 Hz, 1H); 8.46 (broad t, J=6.2 Hz, 1H). LCMS (A): ES m/z=558 [M+2H]²⁺; m/z=1113 [M−H]⁻; m/z=1115 [M+H]⁺; m/z=1159 [M−H+HCO₂H]⁻; t_{R}=0.9 min.

### Example 30: 5-(((S)-1-(((S)-1-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)amino)-1-oxo-6-((2S,3S,4S,5R,6R)-3,4,5-trihydroxy-6-methoxytetrahydro-2H-pyran-2-carboxamido)hexan-2-yl)amino)-3-methyl-1-oxobutan-2-yl)amino)-5-oxopentanoic acid

To compound 69 (47 mg, 42.1 μmol) was added DMF (4 mL) and, under magnetic stirring, glutaric anhydride (8.5 mg, 74.5 μmol); the reaction medium was stirred at RT for 2 h. At this time, the crude medium was concentrated in vacuo, and purified by 2 consecutive flash chromatographies on silica gel (gradient elution DCM/MeOH/H₂O and DCM/MeOH/NH₄OH) to give 17 mg of example 30 (33%).

RMN ¹H (500 MHz, δ in ppm, DMSO-d6): 0.81 (split d, J=6.4 Hz, 6H); 0.84 (d, J=6.6 Hz, 3H); 0.86 (d, J=6.6 Hz, 3H); 0.98 (s, 3H); 1.04 (d, J=6.6 Hz, 3H); 1.11 (s, 3H); 1.17 to 2.32 (broad m, 18H); 2.66 to 2.74 (m, 2H); 2.91 to 3.03 (m, 5H); 3.10 (m large, 1H); 3.18 (t, J=8.8 Hz, 1H); 3.28 to 3.36 (partially masked m, 2H); 3.37 (s, 3H); 3.60 (m, 1H); 3.81 (s, 3H); 3.87 (d, J=1.6 Hz, 1H); 4.07 (m, 2H); 4.14 to 4.26 (m, 3H); 4.31 (m, 1H); 4.92 (m, 1H); 5.06 to 5.13 (m, 2H); 5.35 (m, 2H); 5.80 (d, J=15.3 Hz, 1H); 6.45 (ddd, J=3.6, 11.1 and 15.3 Hz, 1H); 7.05 (d, J=8.5 Hz, 1H); 7.18 (dd, J=2.1 and 8.5 Hz, 1H); 7.22 (d, J=8.4 Hz, 2H); 7.25 (d, J=8.4 Hz, 2H); 7.29 (d, J=2.1 Hz, 1H); 7.31 (m, 1H); 7.77 to 8.81 (m, 5H). LCMS (A): ES m/z=1227 [M−H]⁻; m/z=1229 [M+H]⁺; m/z=615; t_{R}=1.16 min.

### Example 31: 2,5-dioxopyrrolidin-1-yl 5-(((S)-1-(((S)-1-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)amino)-1-oxo-6-((2S,3S,4S,5R,6R)-3,4,5-trihydroxy-6-methoxytetrahydro-2H-pyran-2-carboxamido)hexan-2-yl)amino)-3-methyl-1-oxobutan-2-yl)amino)-5-oxopentanoate

To example 30 (14 mg, 11.4 μmol), under magnetic stirring, were added DMF (3 mL), DSC (4.2 mg, 15.6 μmol) and DIEA (6 μL, 34.5 μmol). The reaction medium was stirred at RT for 4 h. At this time, the medium was diluted with MeTHF (10 mL) and washed with H₂O (5 mL). The aqueous phase was extracted with MeTHF (10 mL), the combined organic phases were dried over MgSO₄, filtered concentrated in vacuo and purified by flash chromatography on 4 g of silica gel (gradient elution DCM/iPrOH) to give 3.5 mg of example 31 (23%).

RMN ¹H (500 MHz, δ in ppm, DMSO-d6): 0.78 (d, J=6.6 Hz, 6H); 0.82 (d, J=7.1 Hz, 3H); 0.84 (d, J=7.1 Hz, 3H); 0.99 (s, 3H); 1.04 (d, J=6.6 Hz, 3H); 1.12 (s, 3H); 1.17 to 1.70 (m, 9H); 1.77 to 1.87 (m, 3H); 1.98 (m, 1H); 2.23 to 2.32 (m, 3H); 2.65 to 2.70 (m, 4H); 2.81 (s, 4H); 2.93 to 3.07 (m, 6H); 3.15 (m, 1H); 3.28 to 3.38 (partially masked m, 2H); 3.40 (s, 3H); 3.53 (d, J=9.6 Hz, 1H); 3.81 (s, 3H); 3.87 (d, J=1.4 Hz, 1H); 4.09 (d, J=7.7 Hz, 1H); 4.17 (dd, J=6.9 and 8.2 Hz, 1H); 4.20 to 4.33 (m, 4H); 4.90 (dd, J=3.4 and 9.5 Hz, 1H); 5.04 (m, 2H); 5.10 (m, 1H); 5.14 (d, J=5.2 Hz, 1H); 5.79 (dd, J=0.8 and 15.3 Hz, 1H); 6.47 (m, 1H); 7.05 (d, J=8.8 Hz, 1H); 7.17 (dd, J=1.6 and 8.8 Hz, 1H); 7.20 to 7.25 (m, 5H); 7.28 (d, J=1.6 Hz, 1H); 7.86 to 7.99 (m, 3H); 8.37 (m, 2H). LCMS (A): ES m/z=1370 [M−H+HCO₂H]⁻; ES m/z=1326 [M+H]⁺; m/z=663.5; t_{R}=1.21 min.

### Example 32: hu2H11_R35-74-Ex31

The general method described previously was used for the preparation of example 32. 27.9 mg of hu2H11_R35-74 were reacted with 107 μL of a 10.5 mM solution of example 31 in DMA (6 eq.) for 2 h 30 then were added 107 μL of a 10.5 mM solution of example 31 in DMA (6 eq.) for 4 h 30. After purification on Superdex 200 μg in buffer B pH 6.5+10% NMP, concentration on Amicon Ultra-15, dilution in buffer B pH 6.5 to a final concentration of NMP at 5% and filtration on 0.22 μm PVDF filter, 16.7 mg of example 32 were obtained as a colorless limpid solution at a concentration of 2.23 mg/mL with a DAR of 3 (HRMS), a monomeric purity of 100% and a global yield of 60%.

SEC-HRMS: m/z=149407 (naked mAb); m/z=150619 (D1); m/z=151831 (D2); m/z=153044 (D3); m/z=154256 (D4); m/z=155468 (D5); m/z=156685 (D6); m/z=157899 (D7); m/z=159014 (D8).

### Synthesis of Examples 33 to 35: glutaryl-Val-sulfoPEG4Lys-C52 benzylic amine, NHS ester of glutaryl-Val-sulfoPEG4Lys-C52 benzylic amine and Corresponding ADC

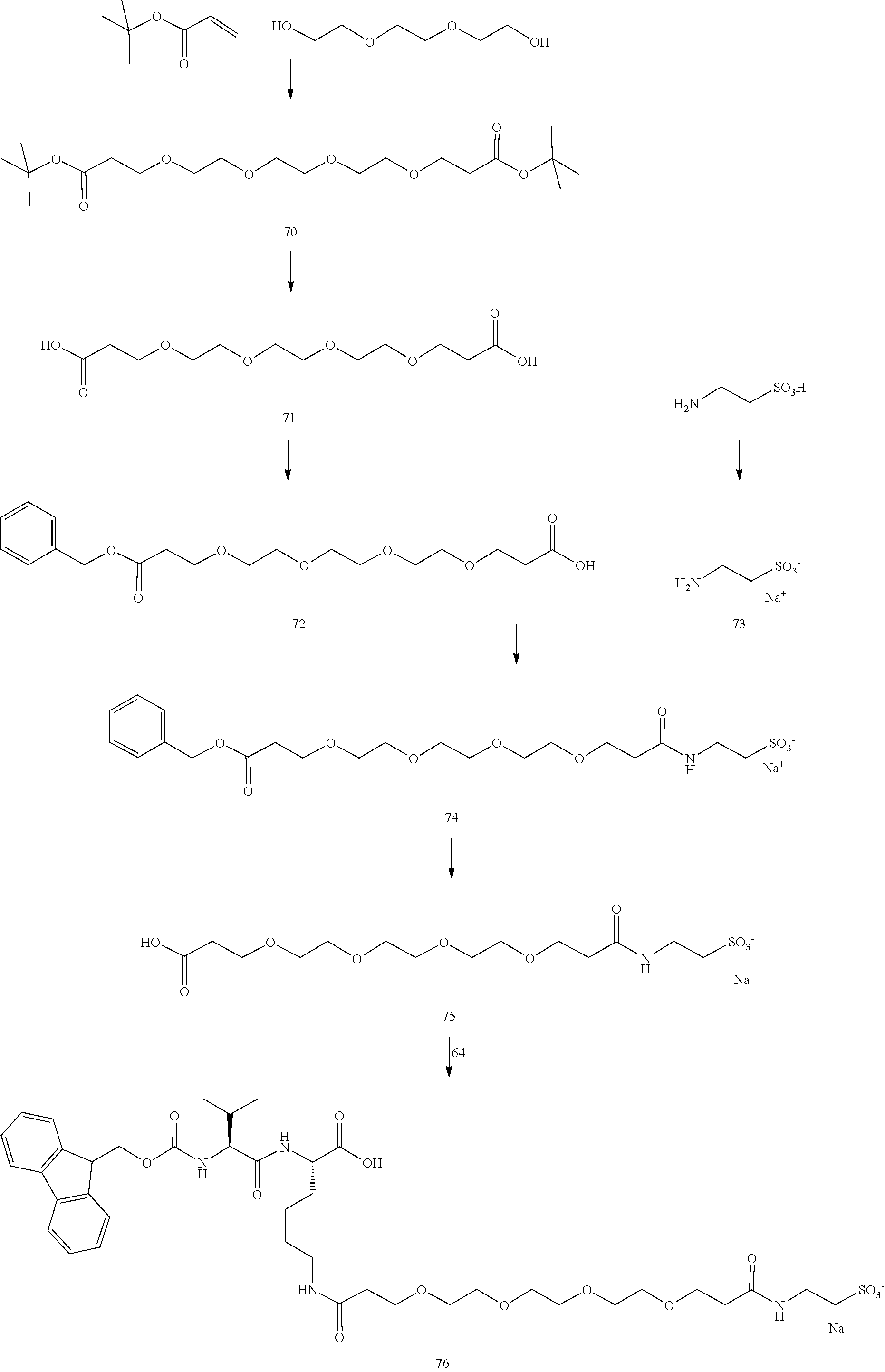

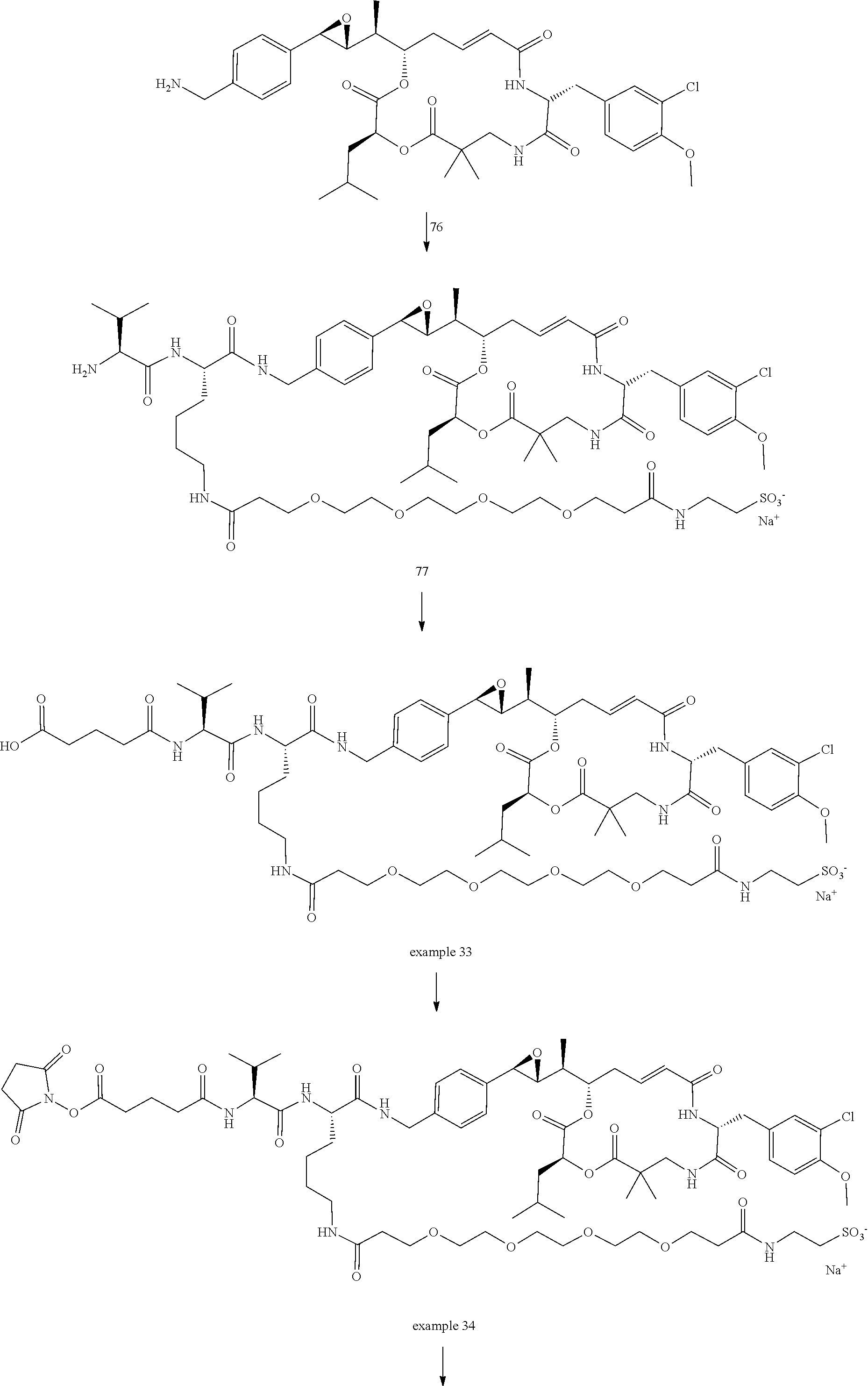

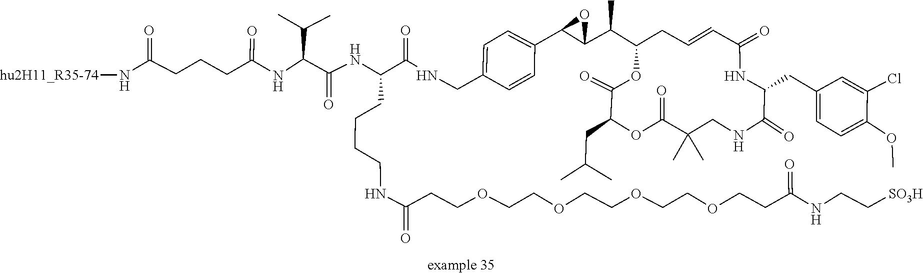

### Compound 70: di-tert-butyl 4,7,10,13-tetraoxahexadecane-1,16-dioate

To a solution of triethylene glycol (5 mL, 37.5 mmol) in THF (15 mL) was added sodium (18 mg, 783.0 μmol). The reaction medium was stirred for 2 h at RT then was added dropwise over 5 min tert-butyl acrylate (14 mL, 95.58 mmol). The reaction medium was stirred for 20 h at RT then quenched with brine (300 mL) and extracted twice with EtOAc (300 mL). The combined organic phases were dried over MgSO₄, filtered, concentrated in vacuo and purified by flash chromatography on 200 g of silica gel (gradient elution heptane/EtOAc) to five 8.05 g of compound 70 as a colorless oil (53%).

RMN ¹H (400 MHz, δ in ppm, DMSO-d6): 1.40 (s, 18H); 2.40 (t, J=6.5 Hz, 4H); 3.48 (m, 12H); 3.58 (t, J=6.5 Hz, 4H). LCMS (A): ES m/z=407 [M+H]⁺; m/z=429 [M+Na]⁺; t_{R}=1.32 min.

### Compound 71: 4,7,10,13-tetraoxahexadecane-1,16-dioic acid

To compound 70 (8.05 g, 19.8 mmol) was added formic acid (40 mL, 1.04 mol). The reaction medium was stirred for 1 d at RT, concentrated in vacuo and co-evaporated with EtOAc (20 mL) to give 5.9 g of compound 71 as a colorless oil (quant.).

RMN ¹H (300 MHz, δ in ppm, DMSO-d6): 2.44 (t, J=6.5 Hz, 4H); 3.48 (s, 8H); 3.50 (s, 4H); 3.59 (d, J=6.5 Hz, 4H); 12.25 (broad m, 2H).

### Compound 72: 3-oxo-1-phenyl-2,6,9,12,15-pentaoxaoctadecan-18-oic acid

To a solution of compound 71 (5.9 g, 20.05 mmol) in DMF (50 mL) were added DIEA (3.6 mL, 20.67 mmol) and dropwise over 10 min a solution of benzyl bromide (2.43 mL, 20.05 mmol) in DMF (50 mL). The reaction medium was stirred for 20 h at RT, quenched with 1N HCl (500 mL) and extracted twice with EtOAc (200 mL). The combined organic phases were dried over MgSO₄, filtered, concentrated in vacuo and purified by two consecutive flash chromatographies on 400 g and 100 g of silica gel (gradient elution DCM/MeOH) to give 2.7 g of compound 72 as a pale green oil (35%).

RMN ¹H (300 MHz, δ in ppm, DMSO-d6): 2.42 (m, 2H); 2.60 (t, J=6.5 Hz, 2H); 3.47 (s, 6H); 3.49 (s, 6H); 3.59 (m, 2H); 3.66 (d, J=6.5 Hz, 2H); 5.10 (s, 2H); 7.29 to 7.41 (m, 5H).

### Compound 73: sodium 2-aminoethanesulfonate

To a solution of taurine (2 g, 15.98 mmol) in H₂O (30 mL) was added sodium hydrogenocarbonate (1.34 g, 15.98 mmol). The reaction medium was stirred for 2 h at RT then concentrated in vacuo. The crude product was dissolved in H₂O (100 mL) and lyophilized to give 2.5 g of compound 73 as a white powder (quant.).

IR spectrum as a KBr pellet; main absorption bands in reciprocal centimeters: 1458; 1344; 1212; 1184; 1046; 1037; 742; 737; 598; 576; 533 & 523.

### Compound 74: sodium 3,18-dioxo-1-phenyl-2,6,9,12,15-pentaoxa-19-azahenicosane-21-sulfonate

To a solution of compound 72 (1 g, 2.69 mmol) in DMF (40 mL) were added NHS (367 mg, 3.13 mmol) and EDC (565 μL, 3.12 mmol). The reaction medium was stirred for 20 h at RT then were added a solution of compound 73 (1.9 g, 12.91 mmol) in H₂O (20 mL) and sodium bicarbonate (219 mg, 2.61 mmol). The reaction medium was stirred for 2 h at RT then acidified to pH 3 with Amberlite IR-120 (H) (CAS number [78922-04-0], filtered, concentrated in vacuo and purified by flash chromatography on 150 g of silica gel (DCM/MeOH/H₂O) to give 824 mg of compound 74 as a colorless solid 61%).

LCMS (A): ES m/z=490 [M−H]⁻; m/z=492 [M+H]⁺; t_{R}=1.1 min.

### Compound 75: sodium 1-carboxy-15-oxo-3,6,9,12-tetraoxa-16-azaoctadecane-18-sulfonate

A solution of compound 74 (820 mg, 1.60 mmol) in 8:2 MeOH/H₂O (30 mL) was filtered over 0.22 μm and subjected to hydrogenolysis on a 10% Pd/C cartridge using a H-cube system at 20° C. The reaction medium was concentrated in vacuo to give 530 mg of compound 75 as a yellow gum (78%).

RMN ¹H (300 MHz, δ in ppm, DMSO-d6): 2.27 (t, J=6.6 Hz, 2H); 2.40 (t, J=6.6 Hz, 2H); 2.52 (partially masked m, 2H); 3.28 (partially masked m, 2H); 3.49 (m, 12H); 3.59 (m, 4H); 7.72 (broad t, J=6.4 Hz, 1H); 12.08 (broad m, 1H).

### Compound 76: sodium (5S,8S)-8-carboxy-1-(9H-fluoren-9-yl)-5-isopropyl-3,6,14,29-tetraoxo-2,17,20,23,26-pentaoxa-4,7,13,30-tetraazadotriacontane-32-sulfonate

To a solution of compound 75 (300 mg, 708.5 μmol) in DMF (15 mL) were added NHS (98 mg, 851.5 μmol) and EDC (150 μL, 855.1 μmol). The reaction medium was stirred for 20 h at RT then were added a solution of compound 64 (416 mg, 825.4 μmol) in DMF (15 mL) and a solution of sodium biacarbonate (180 mg, 2.13 mmol) in H₂O (10 mL). The reaction medium was stirred for 1 h at RT, acidified to pH 3 with Amberlit IR-120 (H) (CAS number [78922-04-0]), filtered, washed with DMF (5 mL), concentrated in vacuo and purified by flash chromatography on 100 g of silica gel (gradient elution DCM/MeOH/acetic acid) to give 447 mg of a colorless lacquer. This lacquer was dissolved in 2:1 THF/H₂O (120 mL), acidified to pH 3.3 with 0.1 N HCl (about 4 mL) and concentrated in vacuo to give 354 mg of compound 76 as a white translucent solid (57%).

RMN ¹H (400 MHz, δ in ppm, DMSO-d6): 0.86 (d, J=7.0 Hz, 3H); 0.89 (d, J=7.0 Hz, 3H); 1.20 to 1.41 (m, 4H); 1.50 to 1.74 (m, 2H); 1.98 (m, 1H); 2.26 (t, J=6.8 Hz, 2H); 2.28 (t, J=6.8 Hz, 2H); 2.52 (masked m, 2H); 3.00 (m, 2H); 3.28 (partially masked m, 2H); 3.45 (m, 12H); 3.58 (t, J=6.8 Hz, 4H); 3.91 (dd, J=7.0 and 9.2 Hz, 1H); 4.14 (m, 1H); 4.19 to 4.31 (m, 3H); 7.32 (m, 2H); 7.37 (d, J=9.2 Hz, 1H); 7.41 (dt, J=1.0 and 7.8 Hz, 2H); 7.75 (m, 3H); 7.80 (t, J=6.0 Hz, 1H); 7.89 (d, J=7.8 Hz, 2H); 8.07 (d, J=8.0 Hz, 1H); 12.49 (m, 1H). LCMS (A): ES m/z=849 [M−H]⁻; m/z=851 [M+H]⁺; t_{R}=1.44 min.

### Compound 77: sodium (25S,28S)-28-amino-25-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclo-hexadec-13-en-16-yl)ethyl)oxiran-2-yl)benzyl)carbamoyl)-29-methyl-4,19,27-trioxo-7,10,13,16-tetraoxa-3,20,26-triazatriacontane-1-sulfonate

To a solution (E)-(3S,10R,16S)-16-{(S)-1-[(2R,3R)-3-(4-aminomethyl-phenyl)-oxiranyl]-ethyl}-10-(3-chloro-4-methoxy-benzyl)-3-isobutyl-6,6-dimethyl-1,4-dioxa-8,11-diaza-cyclohexadec-13-ene-2,5,9,12-tetraone (the synthesis of which was described in WO2011001052, compound 77,106 mg, 151.8 μmol) in DMF (5 mL) were added a solution of compound 76 (225 mg, 257.7 μmol) in DMF (5 mL), HOBt (30 mg, 213.1 μmol) and EDC (40 μL, 226.0 μmol). The reaction medium as stirred for 2 h at RT then was added piperidine (134 μL, 1.36 mmol) and the stirring was carried on for 2 h. The reaction medium was concentrated in vacuo and purified by two consecutive flash chromatographies on 50 g and 4 g of silica gel (gradient elution DCM/MeOH/H₂O) to give 73 mg of compound 77 as a white solid (36%).

RMN ¹H (500 MHz, δ in ppm, DMSO-d6): 0.78 (d, J=6.8 Hz, 6H); 0.91 (d, J=7.0 Hz, 3H); 0.93 (d, J=7.0 Hz, 3H); 1.00 (s, 3H); 1.04 (d, J=7.1 Hz, 3H); 1.12 (s, 3H); 1.18 to 1.45 (m, 5H); 1.51 to 1.72 (m, 4H); 1.80 (m, 1H); 2.05 (m, 1H); 2.23 to 2.35 (m, 5H); 2.56 (m, 2H); 2.68 (m, 2H); 2.95 to 3.04 (m, 5H); 3.32 (m, 3H); 3.48 (m, 12H); 3.57 (t, J=6.7 Hz, 2H); 3.59 (t, J=6.7 Hz, 2H); 3.66 (d, J=6.0 Hz, 1H); 3.80 (s, 3H); 3.87 (d, J=2.3 Hz, 1H); 4.20 to 4.36 (m, 4H); 4.90 (dd, J=3.8 and 9.8 Hz, 1H); 5.12 (m, 1H); 5.80 (dd, J=2.0 and 15.2 Hz, 1H); 6.47 (ddd, J=3.8, 11.3 and 15.2 Hz, 1H); 7.05 (d, J=8.6 Hz, 1H); 7.16 (dd, J=2.4 and 8.6 Hz, 1H); 7.24 (m, 5H); 7.29 (d, J=2.4 Hz, 1H); 7.76 (t, J=6.0 Hz, 1H); 7.86 (t, J=8.0 Hz, 1H); 7.93 (broad m, 3H); 8.38 (d, J=8.0 Hz, 1H); 8.49 (d, J=8.0 Hz, 1H); 8.59 (t, J=6.0 Hz, 1H).

LCMS (A): ES m/z=655 [M+2H]²⁺; m/z=1306 [M−H]⁻; m/z=1308 [M+H]⁺; t_{R}=1.07 min.

### Example 33: sodium (25S,28S)-33-carboxy-25-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclo-hexadec-13-en-16-yl)ethyl)oxiran-2-yl)benzyl)carbamoyl)-28-isopropyl-4,19,27,30-tetraoxo-7,10,13,16-tetraoxa-3,20,26,29-tetraazatritriacontane-1-sulfonate

To a solution of compound 77 (73 mg, 54.9 μmol) in DMF was added glutaric anhydride (11 mg, 96.4 μmol). The reaction medium was stirred for 20 h at RT, concentrated in vacuo and purified by flash chromatography on 4 g of silica gel (DCM/MeOH/H₂O) to give 40 mg of example 33 as a white lacquer (50%).

RMN ¹H (400 MHz, δ in ppm, DMSO-d6): 0.81 (s, 9H); 1.04 (d, J=7.0 Hz, 3H); 1.38 (s, 9H); 1.50 (dd, J=3.0 and 14.5 Hz, 1H); 1.58 (dd, J=9.1 and 14.5 Hz, 1H); 2.38 to 2.48 (partially masked m, 3H); 4.04 (m, 1H); 4.19 (m, 2H); 4.30 (m, 1H); 4.49 (s, 2H); 4.91 (m, 1H); 5.80 (d, J=16.0 Hz, 1H); 6.17 (dd, J=8.4 and 16.0 Hz, 1H); 6.43 (d, J=16.0 Hz, 1H); 6.70 (m, 1H); 7.23 to 7.44 (m, 8H); 7.68 (m, 2H); 7.79 (d, J=8.3 Hz, 1H); 7.88 (d, J=7.8 Hz, 2H). LCMS (A): ES m/z=712 [M+2H]²⁺; m/z=1420 [M−H]⁻; m/z=1422 [M+H]⁺; t_{R}=1.57 min.

### Example 34: sodium (25S,28S)-25-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)-34-((2,5-dioxopyrrolidin-1-yl)oxy)-28-isopropyl-4,19,27,30,34-pentaoxo-7,10,13,16-tetraoxa-3,20,26,29-tetraazatetratriacontane-1-sulfonate

To a solution of example 33 (3.57 mg, 2.47 μmol) in DMA (93 μL) were added NHS (27.2 μL, 2.72 μmol) and EDC (111 μL, 2.22 μmol). The reaction medium was stirred for 48 h at RT: LCMS analysis showed that the presence of expected example 34 at 95% with 5% of residual example 33. This solution was used such as for conjugation and the preparation of example 35.

### Example 35: hu2H11_R35-74-Ex34

The general method described previously was used for the preparation of example 35. 11.16 mg of hu2H11_R35-74 were reacted with 53 μL of a 10 mM solution of example 34 in DMA (7 eq.) for 2 h 30 then were added 15 μL of a 10 mM solution of example 34 in DMA (2 eq.) for 2 h 30. After purification on PD-10 and Nap-10 columns in buffer B pH 6.5+5% NMP, 9.5 mg of example 34 were obtained as a colorless limpid solution at a concentration of 1.9 mg/mL with a DAR of 3.8 (HRMS), a monomeric purity of 97.5% and a global yield of 82%.

SEC-HRMS: m/z=149330 (naked mAb); m/z=150744 (D1); m/z=152147 (D2); m/z=153554 (D3); m/z=154959 (D4); m/z=156366 (D5); m/z=157771 (D6); m/z=159172 (D7); m/z=160578 (D8).

### Synthesis of Examples 36 & 37: glutaryl-Val-PEG4Lys-PABA-C52 benzylic amine and NHS ester of glutaryl-Val-PEG4Lys-PABA-C52 benzylic amine

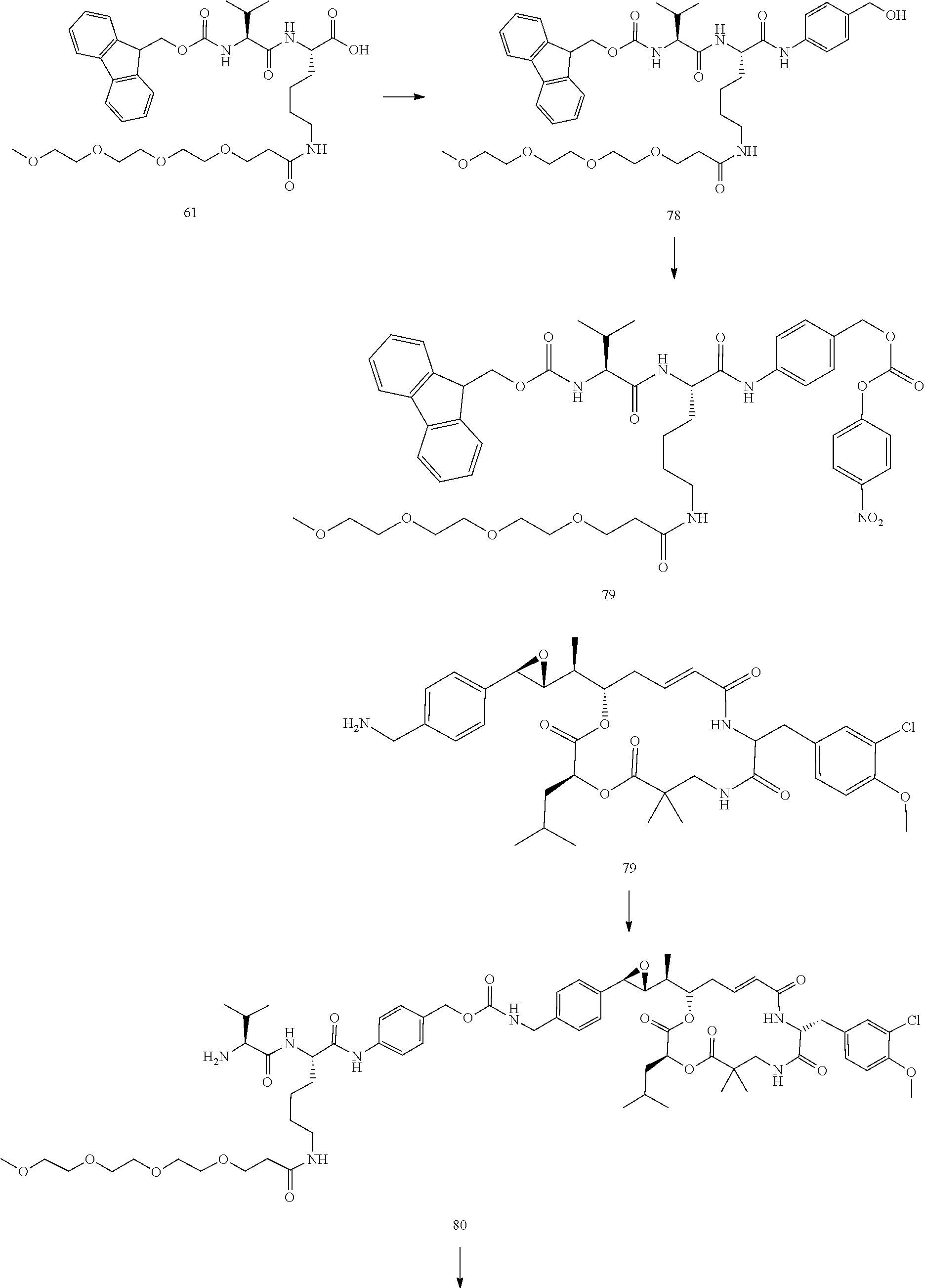

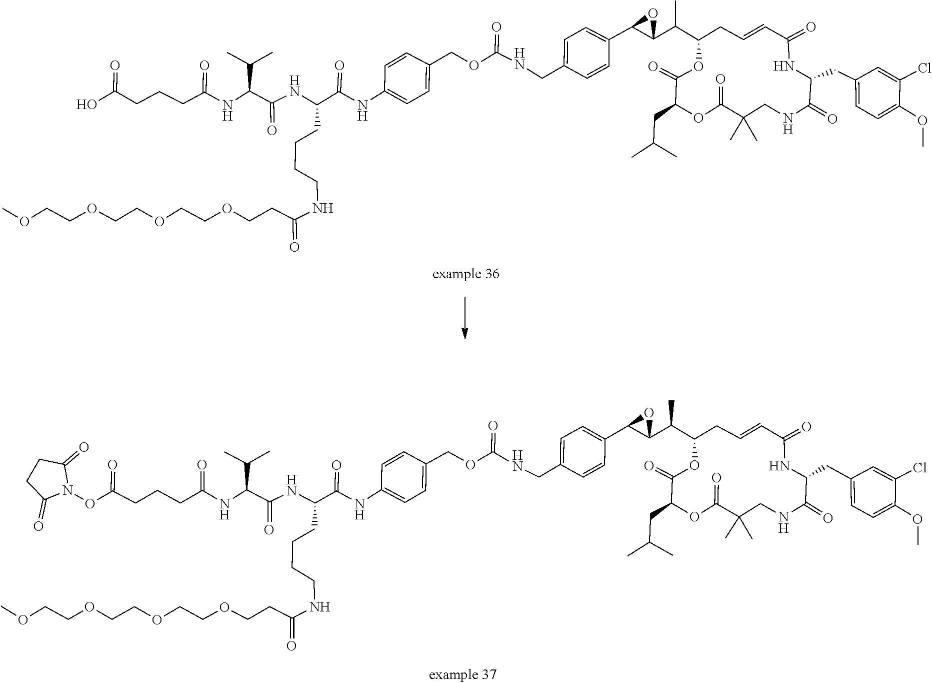

### Compound 78: (9H-fluoren-9-yl)methyl ((20S,23S)-20-((4-(hydroxymethyl)phenyl)carbamoyl)-24-methyl-14,22-dioxo-2,5,8,11-tetraoxa-15,21-diazapentacosan-23-yl)carbamate

To a solution of compound 61 (180 mg, 257.2 μmol) in MeCN (35 mL) were added, under Ar, DIEA (130 μL, 771.7 μmol), 4-aminobenzyl alcohol (48 mg, 385.8 μmol), HOAt (41 mg, 295.8 μmol), HATU (147 mg, 385.8 μmol). The reaction medium was stirred for 17 h at RT then were added MeOH (30 mL) and silica, the mixture was concentrated in vacuo and purified by flash chromatography on 30 g of silica gel (gradient elution DCM/MeOH) to give 94 mg of compound 78 as a beige solid (46%).

RMN ¹H (300 MHz, δ in ppm, DMSO-d6): 0.85 (d, J=7.0 Hz, 3H); 0.88 (d, J=7.0 Hz, 3H); 1.18 to 1.45 (m, 4H); 1.52 to 1.70 (m, 2H); 1.99 (m, 1H); 2.27 (m, 2H); 3.00 (m, 2H); 3.22 (s, 3H); 3.35 to 3.51 (m, 12H); 3.55 (t, J=6.8 Hz, 2H); 3.90 (dd, J=7.2 and 9.1 Hz, 1H); 4.18 to 4.56 (m, 4H); 4.42 (d, J=5.9 Hz, 2H); 5.07 (t, J=5.9 Hz, 1 H); 7.22 (d, J=8.9 Hz, 2H); 7.31 (dt, J=1.8 and 7.8 Hz, 2H); 7.40 (m, 3H); 7.53 (d, J=8.9 Hz, 2H); 7.73 (m, 3H); 7.88 (d, J=7.8 Hz, 2H); 8.02 (d, J=8.2 Hz, 1H); 9.92 (s, 1H).

### Compound 79: (9H-fluoren-9-yl)methyl ((20S,23S)-24-methyl-20-((4-((((4-nitrophenoxy)-carbonyl)oxy)methyl)phenyl)carbamoyl)-14,22-dioxo-2,5,8,11-tetraoxa-15,21-diazapentacosan-23-yl)carbamate

To a solution of compound 78 (94 mg, 118.9 μmol) in DMF were added bis(4-nitrophenyl) carbonate (112 mg, 357.1 μmol) and DIEA (40 μL, 237.2 μmol). The reaction medium was stirred 20 h at RT, concentrated in vacuo, diluted with DCM (10 mL) and washed with H₂O (10 mL). The organic phase was dried over MgSO₄, filtered, concentrated in vacuo and purified by flash chromatography on 12 g of silica gel (gradient elution DCM/MeOH) to give 55 mg of compound 79 as a brown solid (48%).

RMN ¹H (400 MHz, δ in ppm, DMSO-d6): 0.86 (d, J=7.0 Hz, 3H); 0.88 (d, J=7.0 Hz, 3H); 1.18 to 1.45 (m, 4H); 1.55 to 1.77 (m, 2H); 1.99 (m, 1H); 2.27 (t, J=6.8 Hz, 2H); 3.00 (m, 2H); 3.21 (s, 3H); 3.40 to 3.52 (m, 12H); 3.57 (t, J=6.8 Hz, 2H); 3.91 (m, 1H); 4.20 to 4.41 (m, 4H); 5.24 (s, 2H); 7.31 (t, J=7.8 Hz, 2H); 7.35 to 7.48 (m, 5H); 7.58 (d, J=9.2 Hz, 2H); 7.63 (d, J=8.8 Hz, 2H); 7.74 (m, 2H); 7.80 (t, J=6.0 Hz, 1H); 7.90 (d, J=7.8 Hz, 2H); 8.10 (d, J=8.0 Hz, 1H); 8.32 (d, J=9.2 Hz, 2H); 10.12 (s, 1H). LCMS (A): ES m/z=956 [M+H]⁺; m/z=978 [M+Na]⁺; m/z=1000 [M−H+HCO₂H]⁻; t_{R}=1.45 min.

### Compound 80: 4-((S)-20-((S)-2-amino-3-methylbutanamido)-14-oxo-2,5,8,11-tetraoxa-15-azahenicosanamido)benzyl 4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16yl)ethyl)-oxiran-2-yl)benzylcarbamate

To a solution (E)-(3S,10R,16S)-16-{(S)-1-[(2R,3R)-3-(4-aminomethyl-phenyl)-oxiranyl]-ethyl}-10-(3-chloro-4-methoxy-benzyl)-3-isobutyl-6,6-dimethyl-1,4-dioxa-8,11-diaza-cyclohexadec-13-ene-2,5,9,12-tetraone (the synthesis of which was described in WO2011001052, compound 77.19 mg, 27.2 μmol) in DCM (2.5 mL) were added a solution of compound 79 (58 mg, 60.7 μmol) in DCM (2.5 mL) and DIEA (15 μL, 86.6 μmol). The reaction medium was stirred for 20 h at RT, concentrated in vacuo and purified by flash chromatography on 4 g of silica gel (gradient elution DCM/MeOH) to give 38 mg of Fmoc-protected intermediate as a colorless lacquer (92%).

RMN ¹H (400 MHz, δ in ppm, DMSO-d6): 0.79 (d, J=6.7 Hz, 6H); 0.86 (d, J=7.0 Hz, 3H); 0.88 (d, J=7.0 Hz, 3H); 1.00 (s, 3H); 1.03 (d, J=7.0 Hz, 3H); 1.12 (s, 3H); 1.20 to 1.45 (m, 5H); 1.50 to 1.75 (m, 4H); 1.80 (m, 1H); 2.00 (m, 1H); 2.27 (m, 3H); 2.69 (m, 2H); 2.92 to 3.04 (m, 5H); 3.21 (s, 3H); 3.32 (masked m, 1H); 3.35 to 3.51 (m, 12H); 3.56 (t, J=6.6 Hz, 2H); 3.80 (s, 3H); 3.87 (d, J=2.2 Hz, 1H); 3.91 (dd, J=7.1 and 9.1 Hz, 1H); 4.15 to 4.42 (m, 7H); 4.90 (dd, J=3.7 and 9.8 Hz, 1H); 4.97 (s, 2H); 5.10 (m, 1H); 5.79 (dd, J=2.0 and 15.2 Hz, 1H); 6.48 (ddd, J=3.8, 11.1 and 15.2 Hz, 1H); 7.04 (d, J=8.6 Hz, 1H); 7.17 (dd, J=2.4 and 8.6 Hz, 1H); 7.20 to 7.34 (m, 10H); 7.41 (m, 3H); 7.58 (d, J=8.6 Hz, 2H); 7.70 to 7.80 (m, 4H); 7.89 (d, J=7.8 Hz, 2H); 8.06 (d, J=7.6 Hz, 1H); 8.35 (d, J=8.3 Hz, 1H); 10.03 (s, 1H). LCMS (A): ES m/z=698; m/z=1514 [M+H]⁺; m/z=1558 [M−H+HCO₂H]⁻; t_{R}=1.56 min.

To a solution of this intermediate (35 mg, 23.1 μmol) in DCM (5 mL) was added piperidine (20 μL, 201.5 μmol). The reaction medium was stirred for 1 h 30 at RT then DMF (1 mL) was added and stirring carried on for 1 h 30. Then was added piperidine (2 μL); the reaction medium was stirred for 1 h at RT, concentrated in vacuo and purified by flash chromatography on 4 g of silica gel (gradient elution DCM/MeOH) to give 25 mg of compound 80 as a colorless lacquer (83%).

RMN ¹H (400 MHz, δ in ppm, DMSO-d6): 0.78 (d, J=6.7 Hz, 6H); 0.82 (d, J=7.0 Hz, 3H); 0.89 (d, J=7.0 Hz, 3H); 1.00 (s, 3H); 1.04 (d, J=7.1 Hz, 3H); 1.11 (s, 3H); 1.22 to 1.48 (m, 5H); 1.51 to 1.75 (m, 6H); 1.80 (m, 1H); 1.98 (m, 1H); 2.28 (m, 3H); 2.69 (m, 2H); 2.92 to 3.05 (m, 7H); 3.15 (m, 1H); 3.21 (s, 3H); 3.32 (masked m, 1H); 3.35 to 3.51 (m, 12H); 3.57 (t, J=6.7 Hz, 2H); 3.80 (s, 3H); 3.87 (d, J=2.2 Hz, 1H); 4.19 (d, J=6.4 Hz, 2H); 4.23 (ddd, J=3.9, 8.3 and 11.8 Hz, 1H); 4.43 (m, 2H); 4.90 (dd, J=3.7 and 9.8 Hz, 1H); 4.98 (s, 2H); 5.10 (m, 1H); 5.80 (dd, J=2.0 and 15.2 Hz, 1H); 6.48 (ddd, J=3.9, 11.3 and 15.2 Hz, 1H); 7.05 (d, J=8.6 Hz, 1H); 7.18 (dd, J=2.4 and 8.6 Hz, 1H); 7.20 to 7.32 (m, 8H); 7.59 (d, J=8.6 Hz, 2H); 7.79 (t, J=6.0 Hz, 1H); 7.82 (t, J=6.4 Hz, 1H); 8.21 (m, 1H); 8.39 (d, J=8.3 Hz, 1H); 10.13 (s, 1H). LCMS (A): ES m/z=646.5; m/z=1292 [M+H]⁺; t_{R}=1.05 min.

### Example 36: (20S,23S)-20-((4-((((4-((2R,3R)-3-((S)-1-((3S,10R,16S,E)-10-(3-chloro-4-methoxy-benzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)oxy)methyl)-phenyl)carbamoyl)-23-isopropyl-14,22,25-trioxo-2,5,8,11-tetraoxa-15,21,24-triazanonacosan-29-oic acid

To a solution of compound 80 (25 mg, 19.3 μmol) in DCM (5 mL) was added glutaric anhydride (4 mg, 19.3 μmol). The reaction medium was stirred for 2 h 30 at RT then were added glutaric anhydride (4 mg, 19.3 μmol) and DMF (0.5 mL) and stirring carried on for 26 h. The reaction medium was concentrated in vacuo and purified by flash chromatography on 4 g of silica gel (gradient elution DCM/MeOH) to give 10 mg of example 36 as a white lacquer (37%).

### Example 37: (20S,23S)-2,5-dioxopyrrolidin-1-yl 20-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)-carbamoyl)oxy)methyl)phenyl)-carbamoyl)-23-isopropyl-14,22,25-trioxo-2,5,8,11-tetraoxa-15,21,24-triazanonacosan-29-oate

To a solution of example 36 (10 mg, 7.11 μmol) in THF (2 mL) were added DSC (2 mg, 7.65 μmol) and DIEA (1.2 μL, 7.26 μmol). The reaction medium was stirred for 20 h at RT, concentrated in vacuo and purified by two consecutive flash chromatographies on 0.6 g and 2 g of silica gel (gradient elution DCM/iPrOH) to give 3.3 mg of example 37 as a white lacquer (31%).

### Synthesis of Examples 38 & 39: glutaryl-Val-PEG24Lys-PABA-C52 benzylic amine and NHS ester of glutaryl-Val-PEG24Lys-PABA-C52 benzylic amine

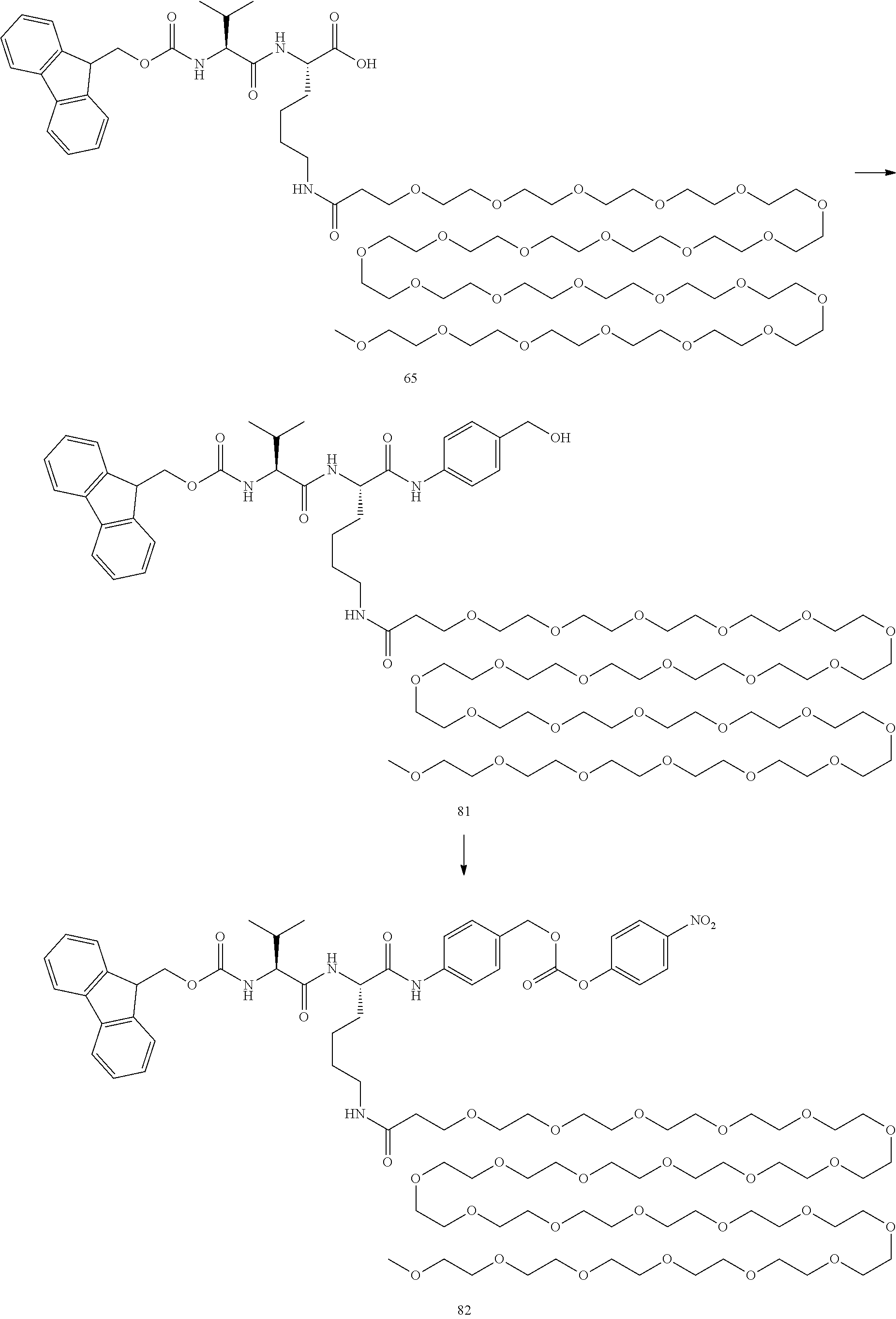 [SMILES: COCCOCCOCCOCCOCCOCCOCCOCCOCCOCCOCCOCCOCCOCCOCCOCCOCCOCCOCCOCCOCCOCCOCCOCCC(=O)NCCCC[C@H](NC(=O)[C@@H](NC(=O)OCC1c2ccccc2-c2ccccc21)C(C)C)C(=O)O ; COCCOCCOCCOCCOCCOCCOCCOCCOCCOCCOCCOCCOCCOCCOCCOCCOCCOCCOCCOCCOCCOCCOCCOCCC(=O)NCCCC[C@H](NC(=O)[C@@H](NC(=O)OCC1c2ccccc2-c2ccccc21)C(C)C)C(=O)Nc1ccc(CO)cc1 ; COCCOCCOCCOCCOCCOCCOCCOCCOCCOCCOCCOCCOCCOCCOCCOCCOCCOCCOCCOCCOCCOCCOCCOCCC(=O)NCCCC[C@H](NC(=O)[C@@H](NC(=O)OCC1c2ccccc2-c2ccccc21)C(C)C)C(=O)Nc1ccc(COC(=O)Oc2ccc([N+](=O)[O-])cc2)cc1]

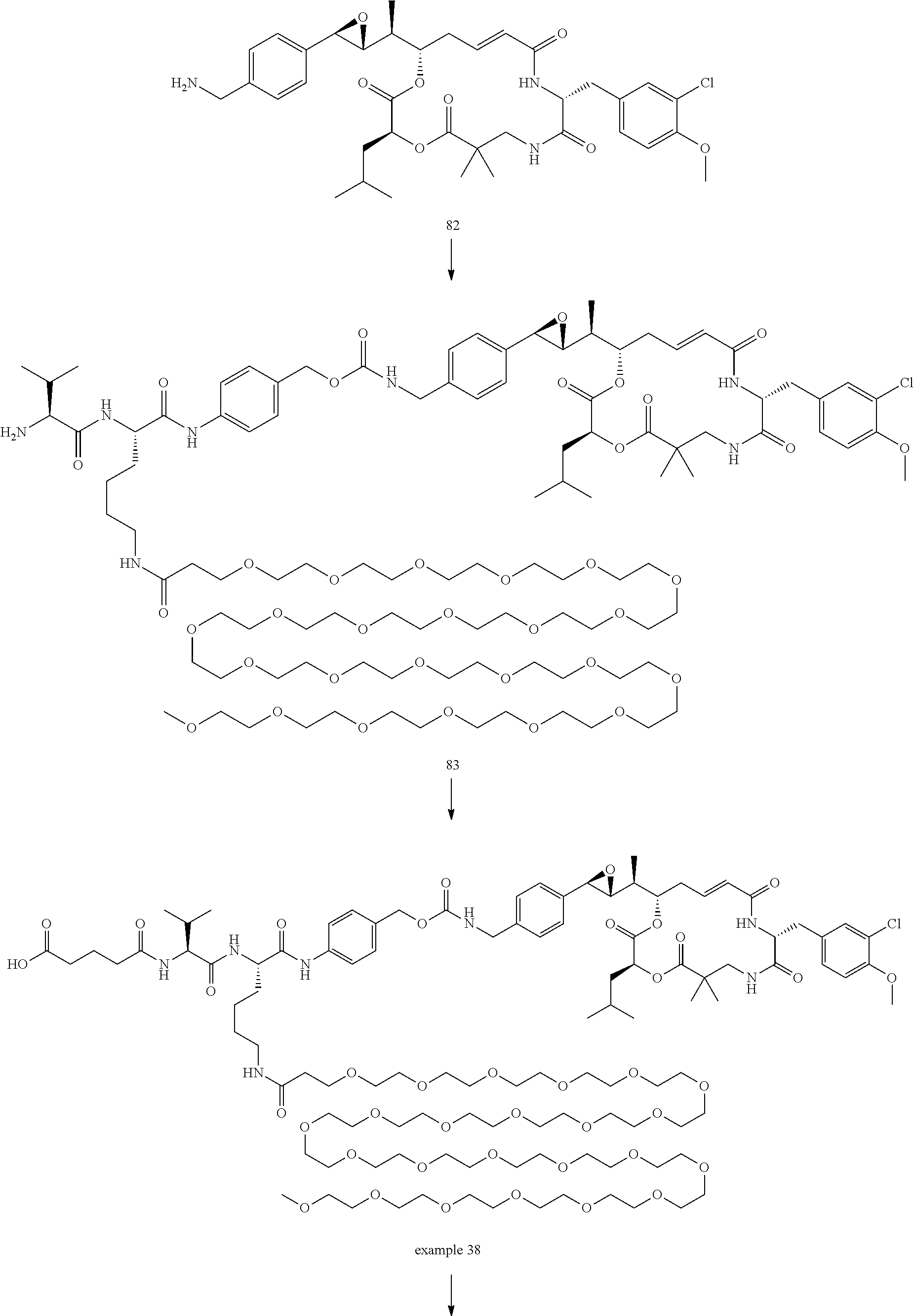

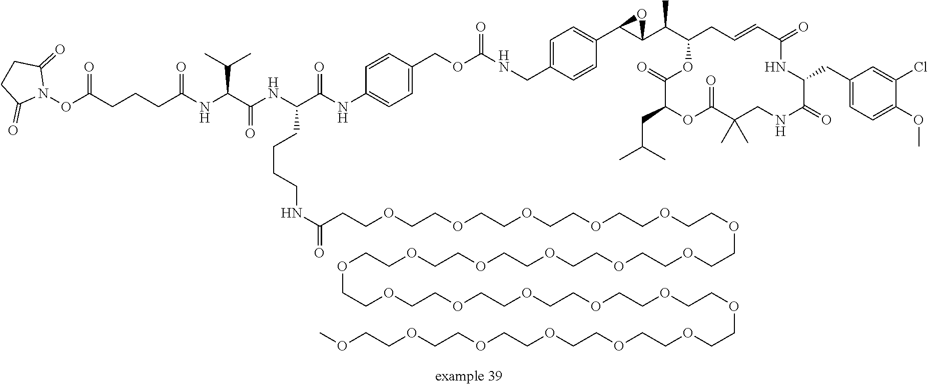 [SMILES: COCCOCCOCCOCCOCCOCCOCCOCCOCCOCCOCCOCCOCCOCCOCCOCCOCCOCCOCCOCCOCCOCCOCCOCCC(=O)NCCCC[C@H](NC(=O)[C@@H](NC(=O)CCCC(=O)ON1C(=O)CCC1=O)C(C)C)C(=O)Nc1ccc(COC(=O)NCc2ccc([C@H]3O[C@@H]3[C@@H](C)[C@@H]3C/C=C/C(=O)N[C@H](Cc4ccc(OC)c(Cl)c4)C(=O)NCC(C)(C)C(=O)O[C@@H](CC(C)C)C(=O)O3)cc2)cc1]

### Compound 81: (9H-fluoren-9-yl)methyl ((80S,83S)-80-((4-(hydroxymethyl)phenyl)carbamoyl)-84-methyl-74,82-dioxo2,5,8,11,14,17,20,23,26, 29,32,35,38,41,44,47,50,53,56,59,62,65,68,71-tetracosaoxa-75,81-diazapentaoctacontan-83-yl)carbamate

To a solution of compound 65 (360 mg, 229.8 μmol) in MeCN (70 mL) were added DIEA (77 μL, 456.6 μmol), 4-aminobenzyl alcohol (43 mg, 342.2 μmol), HOAt (36 mg, 264.5 μmol) and HATU (109 mg, 278.1 μmol). The reaction medium was stirred for 2 h at RT, concentrated in vacuo and purified by flash chromatography on 25 g of silica gel (gradient elution DCM/MeOH/H₂O) to give 307 mg of compound 81 as a white solid (80%).

RMN ¹H (400 MHz, δ in ppm, DMSO-d6): 0.85 (d, J=7.0 Hz, 3H); 0.88 (d, J=7.0 Hz, 3H); 1.21 to 1.42 (m, 4H); 1.54 to 1.78 (m, 2H); 1.99 (m, 1H); 2.27 (t, J=6.8 Hz, 2H); 3.00 (m, 2H); 3.22 (s, 3H); 3.40 to 3.52 (m, 92H); 3.54 (t, J=6.8 Hz, 2H); 3.91 (dd, J=6.9 and 9.0 Hz, 1H); 4.18 to 4.32 (m, 3H); 4.39 (m, 1H); 4.42 (d, J=5.9 Hz, 2H); 5.08 (t, J=5.9 Hz, 1H); 7.22 (d, J=8.9 Hz, 2H); 7.32 (dt, J=1.2 and 7.8 Hz, 2H); 7.41 (m, 3H); 7.53 (d, J=8.9 Hz, 2H); 7.75 (m, 3H); 7.89 (d, J=7.8 Hz, 2H); 8.02 (d, J=8.2 Hz, 1H); 9.92 (s, 1H). LCMS (d): ES m/z=827; m/z=1671 [M+H]⁺; t_{R}=2.94 min.

### Compound 82: (9H-fluoren-9-yl)methyl ((80S,83S)-84-methyl-80-((4-((((4-nitrophenoxy)-carbonyl)oxy)methyl)phenyl)carbamoyl)-74,82-dioxo-2,5,8,11,14,17, 20,23,26,29,32,35,38,41,44,47,50,53,56,59,62,65,68,71-tetracosaoxa-75,81-diazapentaoctacontan-83-yl)carbamate

To a solution of compound 81 (300 mg, 179.4 μmol) in DCM (10 mL) were added bis (4-nitrophenyl) carbonate (450 mg, 1.48 mmol) and DIEA (60 μL, 361.2 μmol). The reaction medium was stirred at RT overnight then was added bis(4-nitrophenyl) carbonate (80 mg, 263.1 μmol) and stirring carried on for 4 h. At that time was added bis(4-nitrophenyl) carbonate (80 mg, 263.1 μmol) and stirring carried on overnight. The reaction medium was concentrated in vacuo and purified by flash chromatography on 80 g of silica gel (gradient elution DCM/MeOH/H₂O) to give 325 mg of compound 82 as a white lacquer (98%).

RMN ¹H (400 MHz, δ in ppm, DMSO-d6): 0.86 (d, J=7.0 Hz, 3H); 0.89 (d, J=7.0 Hz, 3H); 1.20 to 1.45 (m, 4H); 1.58 to 1.76 (m, 2H); 1.99 (m, 1H); 2.27 (t, J=6.8 Hz, 2 H); 3.00 (m, 2H); 3.22 (s, 3H); 3.40 to 3.70 (m, 94H); 3.91 (dd, J=7.2 and 9.1 Hz, 1H); 4.19 to 4.32 (m, 3H); 4.39 (m, 1H); 5.24 (s, 2H); 7.31 (dt, J=1.2 and 7.8 Hz, 2H); 7.40 (m, 4H); 7.57 (d, J=9.2 Hz, 2H); 7.62 (d, J=8.6 Hz, 2H); 7.73 (m, 3H); 7.89 (d, J=7.8 Hz, 2H); 8.07 (d, J=7.8 Hz, 1H); 8.31 (d, J=9.2 Hz, 2H); 10.10 (s, 1H). LCMS (d): ES m/z=827; m/z=1836 [M+H]⁺; t_{R}=3.38 min.

### Compound 83: 4-((S)-80-((S)-2-amino-3-methylbutanamido)-74-oxo-2,5,8,11,14,17,20,23,26,29,32,35,38,41,44,47,50,53,56,59,62,65,68,71-tetracosaoxa-75-azahenoctacontanamido)-benzyl 4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl-carbamate

To a solution of (E)-(3S,10R,16S)-16-{(S)-1-[(2R,3R)-3-(4-aminomethyl-phenyl)-oxiranyl]-ethyl}-10-(3-chloro-4-methoxy-benzyl)-3-isobutyl-6,6-dimethyl-1,4-dioxa-8,11-diaza-cyclohexadec-13-ene-2,5,9,12-tetraone (the synthesis of which was described in WO2011001052, compound 77, 79 mg, 113.1 μmol) in DCM (5 mL) were added a solution of compound 82 (325 mg, 177.5 μmol) in DCM (5 mL) and DIEA (63 μL, 365.6 μmol). The reaction medium was stirred for 20 h at RT then was added compound 82 (34 mg, 18.6 μmol) and stirring carried on for 4 h. The reaction medium was concentrated in vacuo and purified by two consecutive flash chromatographies on 25 g of silica gel and 15 g of diol-modified silica gel (gradient elution DCM/MeOH) to give 152 mg of crude product that was purified by reverse phase chromatography on a 10 μm C18 column 250×40 mm (gradient elution MeCN/H₂O) to give 78 mg of the Fmoc-protected intermediate containing 16% of compound 83 as a white powder (29%).

RMN ¹H (500 MHz, δ in ppm, DMSO-d6): 0.78 (d, J=6.8 Hz, 6H); 0.85 (d, J=7.0 Hz, 3H); 0.88 (d, J=7.0 Hz, 3H); 1.00 (s, 3H); 1.04 (d, J=7.1 Hz, 3H); 1.12 (s, 3H); 1.20 to 1.43 (m, 5H); 1.50 to 1.74 (m, 4H); 1.79 (m, 1H); 1.99 (m, 1H); 2.25 (m, 3H); 2.68 (m, 2H); 2.93 to 3.04 (m, 5H); 3.22 (s, 3H); 3.32 (masked m, 2H); 3.40 to 3.76 (m, 94H); 3.80 (s, 3H); 3.87 (d, J=2.0 Hz, 1H); 3.90 (dd, J=7.5 and 9.1 Hz, 1H); 4.19 (d, J=6.5 Hz, 2H); 4.20 to 4.33 (m, 4H); 4.39 (m, 1H); 4.90 (dd, J=3.9 and 9.9 Hz, 1H); 4.93 (s, 2H); 5.11 (m, 1H); 5.79 (dd, J=1.8 and 15.2 Hz, 1H); 6.47 (ddd, J=3.8, 11.4 et 15.2 Hz, 1H); 7.05 (d, J=8.6 Hz, 1H); 7.17 (dd, J=2.4 and 8.6 Hz, 1H); 7.20 to 7.36 (m, 10H); 7.41 (m, 3H); 7.48 (d, J=8.6 Hz, 2H); 7.70 to 7.80 (m, 5H); 7.89 (d, J=7.8 Hz, 2H); 8.08 (d, J=8.0 Hz, 1H); 8.35 (d, J=8.0 Hz, 1H); 10.04 (s, 1H).

To a solution of this intermediate (75 mg, 31.3 μmol) in DMF (5 mL) was added piperidine (25 μL, 253.2 μmol). The reaction medium was stirred for 1 h 30 at RT, concentrated in vacuo and purified by flash chromatography on 4 g of silica gel (gradient elution DCM/MeOH/H₂O) to give 58 mg of compound 83 as a white lacquer (85%).

RMN ¹H (500 MHz, δ in ppm, DMSO-d6): 0.78 (d, J=6.9 Hz, 9H); 0.88 (d, J=7.0 Hz, 3H); 1.00 (s, 3H); 1.05 (d, J=7.1 Hz, 3H); 1.12 (s, 3H); 1.15 to 1.50 (m, 7H); 1.52 to 1.73 (m, 4H); 1.80 (m, 1H); 1.93 (m, 1H); 2.27 (m, 3H); 2.69 (m, 2H); 2.95 to 3.04 (m, 6H); 3.23 (s, 3H); 3.32 (m, 1H); 3.40 to 3.55 (m, 92H); 3.57 (t, J=6.8 Hz, 2H); 3.80 (s, 3H); 3.87 (d, J=2.2 Hz, 1H); 4.20 (d, J=6.5 Hz, 2H); 4.24 (ddd, J=3.9, 8.2 and 12.0 Hz, 1H); 4.42 (m, 1H); 4.90 (dd, J=3.8 and 9.8 Hz, 1H); 4.98 (s, 2H); 5.10 (m, 1H); 5.79 (d, J=15.8 Hz, 1H); 6.47 (ddd, J=3.8, 6.0 and 11.5 Hz, 1H); 7.05 (d, J=8.6 Hz, 1H); 7.16 (dd, J=2.4 and 8.6 Hz, 1H); 7.20 to 7.31 (m, 8H); 7.58 (d, J=8.6 Hz, 2H); 7.78 (m, 2H); 8.10 (d, J=8.6 Hz, 1H); 8.36 (d, J=8.1 Hz, 1H); 10.10 (s, 1H). LCMS (d): ES m/z=725; m/z=1087 [M+2H]²⁺; t_{R}=1.07 min.

### Example 38: (80S,83S)-80-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)oxy)methyl)-phenyl)carbamoyl)-83-isopropyl-74,82,85-trioxo-2,5,8,11,14,17,20,23,26,29,32,35, 38,41,44,47,50,53,56,59,62,65,68,71-tetracosaoxa-75,81,84-triazanonaoctacontan-89-oic acid

To a solution of compound 83 (58 mg, 26.7 μmol) in DCM (2 mL) was added glutaric anhydride (5.5 mg, 48.2 μmol). The reaction medium was stirred for 3 h at RT, concentrated in vacuo and purified by two consecutive flash chromatographies on 4 g and 12 g of silica gel (gradient elution DCM/MeOH/H₂O) to give 37 mg of example 38 as a white lacquer (61%).

RMN ¹H (500 MHz, δ in ppm, DMSO-d6): 0.79 (d, J=6.8 Hz, 6H); 0.93 (d, J=7.0 Hz, 3H); 0.97 (d, J=7.0 Hz, 3H); 1.00 (s, 3H); 1.04 (d, J=7.1 Hz, 3H); 1.12 (s, 3H); 1.20 to 1.45 (m, 5H); 1.51 to 1.75 (m, 6H); 1.80 (m, 1H); 2.00 (m, 1H); 2.13 to 2.33 (m, 7H); 2.69 (m, 2H); 2.95 to 3.05 (m, 5H); 3.23 (s, 3H); 3.32 (m, 1H); 3.40 to 3.58 (m, 94H); 3.80 (s, 3H); 3.87 (d, J=2.2 Hz, 1H); 4.15 to 4.38 (m, 4H); 4.32 (m, 1H); 4.90 (dd, J=3.8 and 9.8 Hz, 1H); 4.98 (s, 2H); 5.10 (m, 1H); 5.79 (d, J=15.2 Hz, 1H); 6.47 (ddd, J=3.8, 11.4 and 15.2 Hz, 1H); 7.05 (d, J=8.6 Hz, 1H); 7.16 (dd, J=2.4 and 8.6 Hz, 1H); 7.20 to 7.32 (m, 8H); 7.60 (d, J=8.6 Hz, 2H); 7.78 (t, J=6.5 Hz, 1H); 7.90 (broad d, J=8.6 Hz, 2H); 8.20 (m, 1H); 8.38 (d, J=8.6 Hz, 2H); 10.08 (m, 1H); 12.10 (m, 1H). LCMS (d): ES m/z=698; m/z=2287 [M+H]⁺; t_{R}=3.13 min.

### Example 39: (80S,83S)-2,5-dioxopyrrolidin-1-yl 80-((4-((((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diaza-cyclohexadec-13-en-16-yl)ethyl)oxiran-2-yl)benzyl)carbamoyl)oxy)methyl)phenyl)carbamoyl)-83-isopropyl-74,82,85-trioxo2,5,8,11,14,17,20,23,26,29,32,35,38,41,44,47,50,53,56,59,62,65,68,71-tetracosaoxa-75,81,84-triazanonaoctacontan-89-oate

To a solution of example 38 (29 mg, 12.7 μmol) in THF (3 mL) were added DSC (3.6 mg, 13.8 μmol) and DIEA (2.1 μL, 12.9 μmol). The reaction medium was stirred for 20 h at RT, concentrated in vacuo and purified by flash chromatography on 4 g of silica gel (gradient elution DCM/iPrOH) to give 6.9 mg of example 39 as a colorless lacquer (23%).

RMN ¹H (500 MHz, δ in ppm, DMSO-d6): 0.79 (d, J=6.8 Hz, 6H); 0.93 (d, J=7.0 Hz, 3H); 0.97 (d, J=7.0 Hz, 3H); 1.00 (s, 3H); 1.04 (d, J=7.1 Hz, 3H); 1.11 (s, 3H); 1.13 to 1.42 (m, 5H); 1.52 to 1.88 (m, 7H); 1.98 (m, 1H); 2.28 (m, 5H); 2.53 to 2.72 (m, 4H); 2.80 (s, 4H); 2.96 to 3.05 (m, 5H); 3.23 (s, 3H); 3.32 (m, 1H); 3.60 to 3.65 (m, 94H); 3.80 (s, 3H); 3.87 (d, J=2.2 Hz, 1H); 4.15 to 4.28 (m, 4H); 4.33 (m, 1H); 4.90 (dd, J=3.8 and 9.8 Hz, 1H); 4.98 (s, 2H); 5.10 (m, 1H); 5.79 (d, J=15.8 Hz, 1H); 6.47 (ddd, J=3.8, 11.4 and 15.2 Hz, 1H); 7.05 (d, J=8.6 Hz, 1H); 7.17 (dd, J=2.4 and 8.6 Hz, 1H); 7.20 to 7.32 (m, 8H); 7.60 (d, J=8.6 Hz, 2H); 7.78 (m, 2H); 7.90 (d, J=8.5 Hz, 1H); 8.07 (m, 2H); 8.37 (d, J=8.5 Hz, 2H); 9.95 (s, 1H). LCMS (D): ES m/z=698; m/z=2384 [M+H]⁺; t_{R}=3.19 min.

### Synthesis of Examples 40 & 41: DBCO-glutaryl-Val-PEG24Lys-PABA-C52 benzylic amine and Corresponding ADC

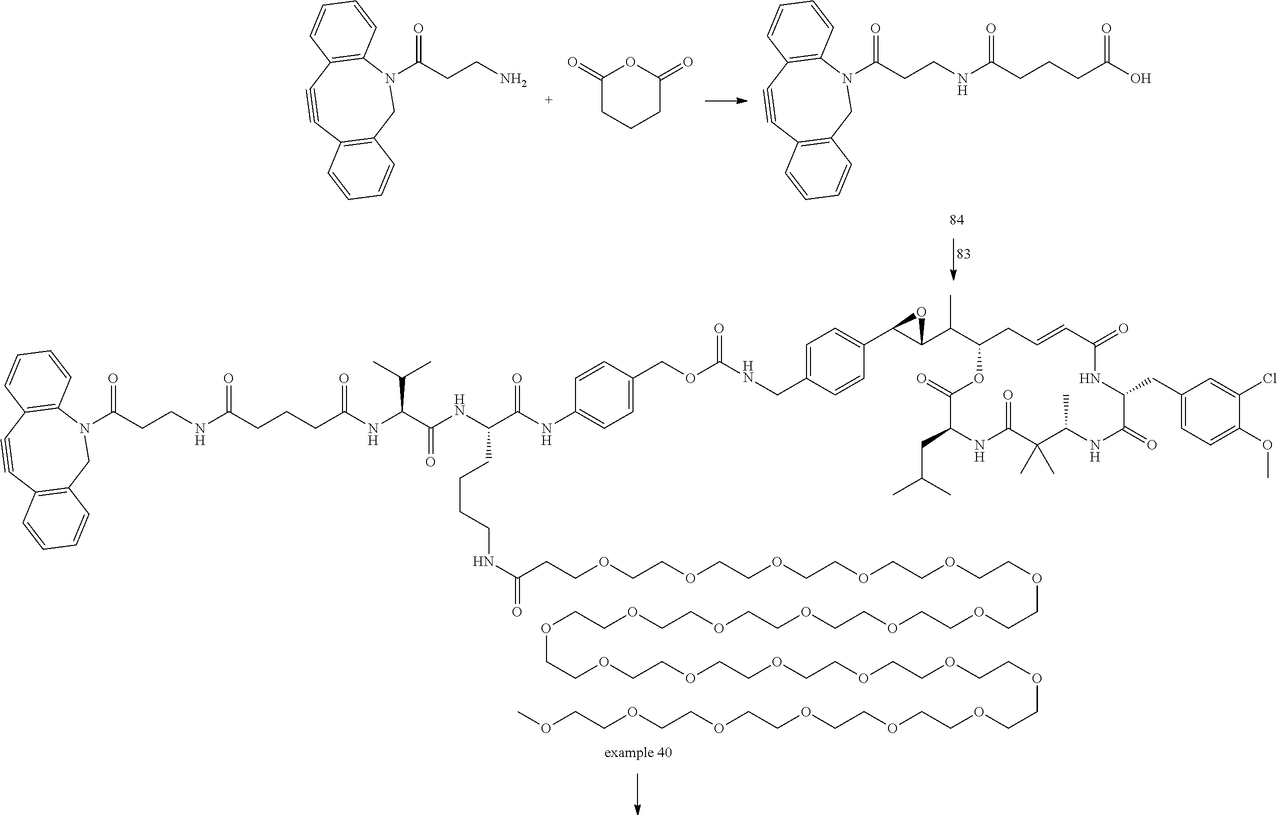 [SMILES: NCCC(=O)N1Cc2ccccc2C#Cc2ccccc21 ; O=C1CCCC(=O)O1 ; O=C(O)CCCC(=O)NCCC(=O)N1Cc2ccccc2C#Cc2ccccc21 ; COCCOCCOCCOCCOCCOCCOCCOCCOCCOCCOCCOCCOCCOCCOCCOCCOCCOCCOCCOCCOCCOCCOCCOCCC(=O)NCCCC[C@H](NC(=O)[C@@H](NC(=O)CCCC(=O)NCCC(=O)N1Cc2ccccc2C#Cc2ccccc21)C(C)C)C(=O)Nc1ccc(COC(=O)NCc2ccc([C@H]3O[C@@H]3C(C)[C@@H]3C/C=C/C(=O)N[C@H](Cc4ccc(OC)c(Cl)c4)C(=O)N[C@@H](C)C(C)(C)C(=O)N[C@@H](CC(C)C)C(=O)O3)cc2)cc1]

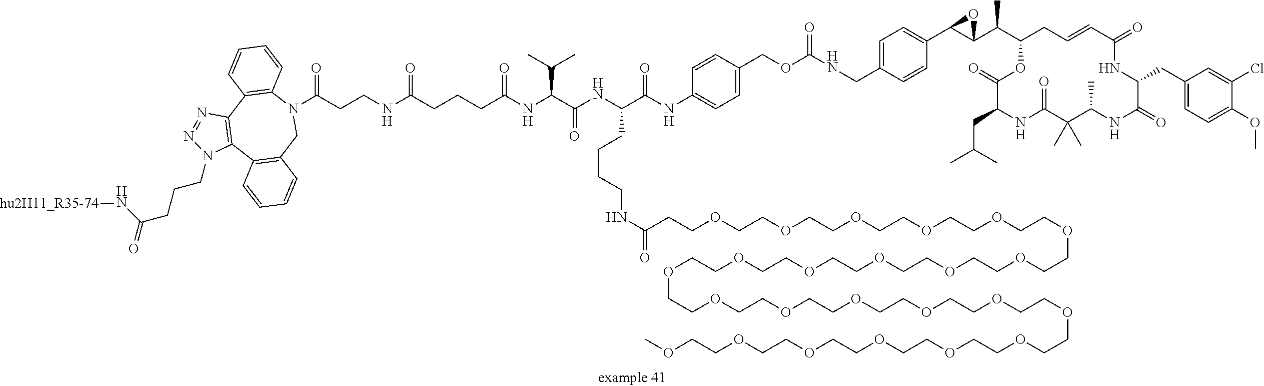

### Compound 84: 5-[[3-(11,12-didehydrodibenz[b,f]azocin-5(6H)-yl)-3-oxopropyl]amino]-5-oxopentanoic acid

To a solution of DBCO-amine (50 mg, 180.9 μmol) in DCM (2 mL) was added glutaric anhydride (21.1 mg, 180.9 μmol). The reaction medium was stirred for 2 h at RT, concentrated in vacuo and purified by flash chromatography on 4 g of silica gel (gradient elution DCM/MeOH) to give 32.6 mg of compound 84 (45%).

RMN ¹H (400 MHz, δ in ppm, DMSO-d6): 1.59 (m, 2H); 1.81 (m, 1H); 1.94 (t, J=7.4 Hz, 2H); 2.11 (t, J=7.4 Hz, 2H); 2.40 (m, 1H); 2.91 (m, 1H); 3.09 (m, 1H); 3.62 (d, J=14.2 Hz, 1H); 5.13 (d, J=14.2 Hz, 1H); 7.38 to 7.53 (m, 6H); 7.56 to 7.67 (m, 3H); 12.05 (broad m, 1H).

### Example 40: DBCO-glutaryl-Val-PEG24Lys-PABA-C52 benzylic amine

To a solution of compound 83 (22 mg, 10.1 μmol) in DCM (2 mL) were added compound 84 (5.14 mg, 13.2 μmol), EDC (1.8 μL, 10.1 μmol) and HOBt (1.4 mg, 10.1 μmol). The reaction medium was stirred at RT overnight, concentrated in vacuo and purified by reverse phase chromatography on a C18-modified 5 μm column 150 mm×30 mm (gradient elution MeCN/0.2N ammonium acetate pH 5.6) to give 9.2 mg of example 40 as a white powder (36%).

RMN ¹H (500 MHz, δ in ppm, DMSO-d6): 0.78 (d, J=6.8 Hz, 6H); 0.80 to 0.90 (m, 6H); 1.00 (s, 3H); 1.05 (d, J=7.1 Hz, 3H); 1.12 (s, 3H); 1.20 to 1.41 (m, 5H); 1.52 to 1.72 (m, 6H); 1.75 to 2.00 (m, 5H); 2.09 (m, 2H); 2.27 (m, 3H); 2.40 (m, 1H); 2.68 (m, 2H); 2.88 to 3.12 (m, 7H); 3.23 (s, 3H); 3.28 to 3.58 (partially masked m, 95H); 3.60 (d, J=11.0 Hz, 1H); 3.80 (s, 3H); 3.88 (d, J=2.0 Hz, 1H); 4.11 to 4.28 (m, 4H); 4.33 (m, 1H); 4.90 (dd, J=3.8 and 9.8 Hz, 1H); 4.97 (s, 2H); 5.04 (d, J=11.0 Hz, 1H); 5.11 (m, 1H); 5.80 (d, J=15.2 Hz, 1H); 6.47 (ddd, J=3.8, 11.4 and 15.2 Hz, 1H); 7.05 (d, J=8.6 Hz, 1H); 7.17 (dd, J=2.4 and 8.6 Hz, 1H); 7.20 to 7.31 (m, 6H); 7.32 to 7.50 (m, 6H); 7.53 to 7.70 (m, 4H); 7.80 (m, 3H); 8.08 (d, J=8.9 Hz, 1H); 8.36 (d, J=8.4 Hz, 1H); 9.95 (s, 1H). LCMS (D): ES m/z=849 [M+3H]³⁺; m/z=1273 [M+2H]²⁺; t_{R}=5.29 min.

### Example 41: hu2H11_R35-74-Ex40

194 mg of hu2H11_R35-74 in buffer A were reacted with 627 μL of a solution of 2,5-dioxopyrrolidin-1-yl 4-azidobutanoate (CAS number [943858-70-6]) at 22.0 mM in DMA (10 eq.) such as the final antibody concentration was 10.5 mg/mL and the percentage of DMA in buffer A was 5%. After stirring for 2 h at RT, the mixture was purified by gel filtration using a a Sephadex™ G25 matrix (Hiprep 26/10 desalting column, GEHealthcare) pre-equilibrated in buffer A. The fractions containing the monomeric modified antibody were pooled and filtered through a Steriflip® filter unit (0.22 μm Durapore® PVDF membrane, Millipore) to provide 173 mg of modified antibody at a concentration of 5.75 mg/mL with a ratio of linker per antibody of 5.2 (HRMS), a monomeric purity of 97% and a global yield of 89%.

The general method described previously was used for the preparation of example 41. 45 mg of modified hu2H11_R35-74 were reacted with 604 μL of a 6.89 mM solution of example 40 in DMA (14 eq.) for 4 h 30 then were added 604 μL of a 10 mM solution of example 40 in DMA (14 eq.) and stirring was pursued overnight at RT. After purification on Superdex 200 μg in buffer B pH 6.5+20% NMP, concentration on Amicon Ultra-15, gel filtration on PD-10 in buffer B pH 6.5+5% NMP and filtration on 0.22 μm PVDF filter, 28 mg of example 41 were obtained as a colorless limpid solution at a concentration of 1.6 mg/mL with a DAR of 3.44 (HRMS), a monomeric purity of 99.6% and a yield of 62%.

SEC-HRMS: m/z=152041 (D1); m/z=154678 (D2); m/z=157332 (D3); m/z=159992 (D4); m/z=162648 (D5); m/z=165303 (D6); m/z=168004 (D7).

### Synthesis of Example 42: sulfo-Val-PEG4Lys-C52 benzylic amine

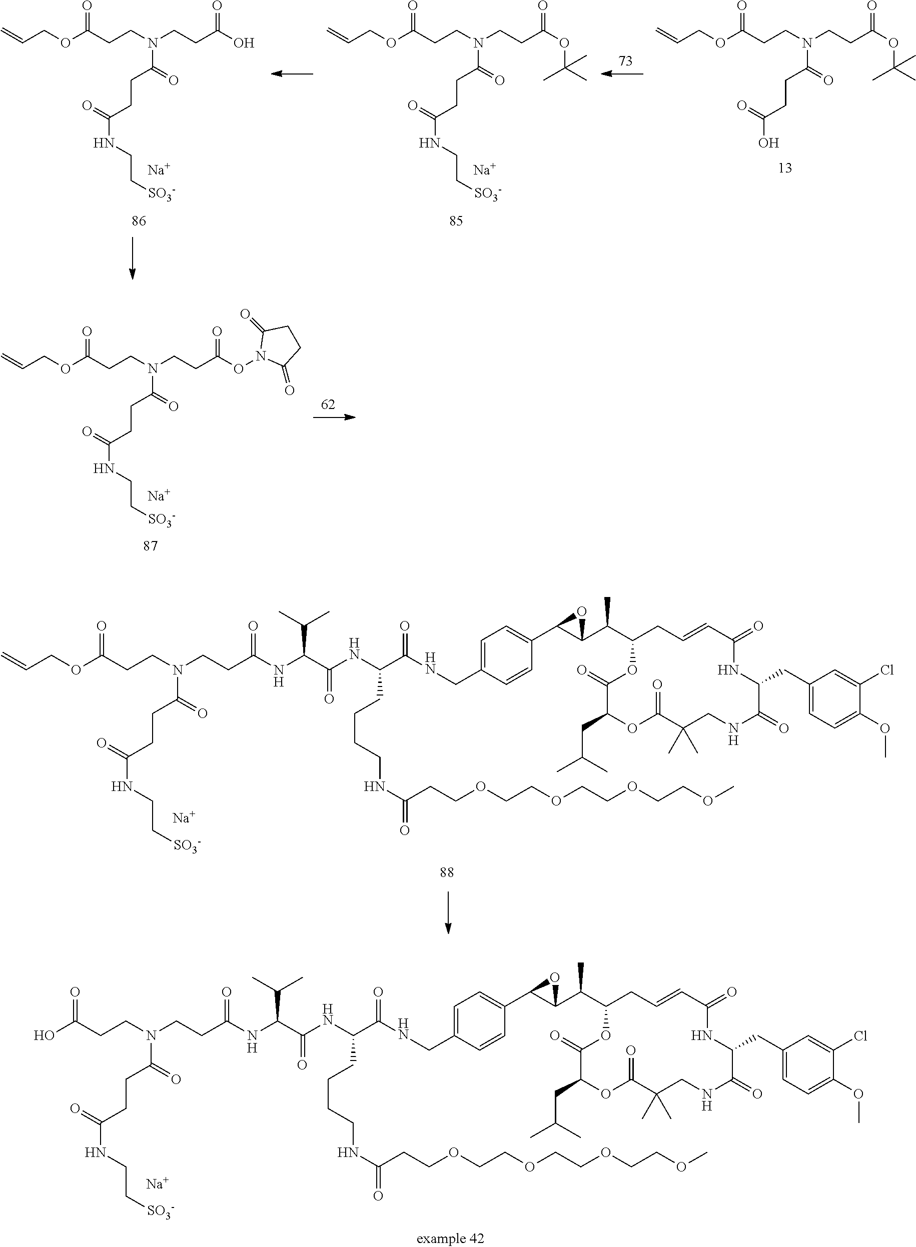

### Compound 85: sodium 2-(4-((3-(allyloxy)-3-oxopropyl)(3-(tert-butoxy)-3-oxopropyl)amino)-4-oxobutanamido)ethane-1-sulfonate

To a solution of compound 13 (0.958 g, 2.68 mmol) in DMF (5 mL) were added NHS (390.3 mg, 3.32 mmol) and EDC (604.2 μL, 3.4 mmol). The reaction medium was stirred for 2 h at RT then stored 2 d at −20° C. After getting back at RT, the reaction medium was stirred for 2 h at RT then were added EDC (60 μL, 340 μmol) and a solution of NHS (20 mg, 170.3 μmol) in DMF (3 mL). The reaction medium was stirred at RT overnight then were added a solution of compound 73 (1.96 g, 13.3 mmol) in H₂O (10 mL) and sodium hydrogenocarbonate (225.2 mg, 2.68 mmol). The reaction medium was stirred for 1 h at RT, diluted with H₂O (6 mL), acidified to pH 3 with Amberlite IR-120 (H) (CAS number [78922-04-4]), filtered, concentrated in vacuo and purified by flash chromatography on silica gel (gradient elution DCM/MeOH/H₂O) to give 270 mg of compound 85 as a colorless foam (21%).

RMN ¹H (400 MHz, δ in ppm, DMSO-d6): 50:50 conformer mixture; 1.39 (s, 4.5H); 1.40 (s, 4.5H); 2.27 (t, J=7.0 Hz, 4H); 2.38 (t, J=7.0 Hz, 2H); 3.53 (m, 3H); 2.68 (t, J=7.0 Hz, 1H); 3.25 to 3.61 (m, 6H); 4.55 to 4.58 (m, 2H); 5.18 to 5.36 (m, 2H); 5.90 (m, 1H); 7.70 (t, J=6.8 Hz, 1H); 10.53 (broad m, 1H). LCMS (A): ES m/z=409; m/z=463 [M−H]⁻; m/z=465 [M+H]⁺; t_{R}=1.13 min.

### Compound 86: sodium 2-(4-((3-(allyloxy)-3-oxopropyl)(2-carboxyethyl)amino)-4-oxobutanamido)-ethane-1-sulfonate

To a solution of compound 85 (60 mg, 123.3 μmol) in DCM (4 mL) was added TFA (100 μL, 1.25 mmol). The reaction medium was stirred for 2 h at RT then was added TFA (100 μL, 1.25 mmol) and stirring was carried on for 2 d at RT. Then was added TFA (50 μL, 0.625 mmol), the reaction medium was stirred at RT, concentrated in vacuo and co-evaporated with toluene (3×) to give 60 mg of compound 86 as a colorless lacquer (quant.).

### Compound 87: sodium 2-(4-((3-(allyloxy)-3-oxopropyl)(3-((2,5-dioxopyrrolidin-1-yl)oxy)-3-oxopropyl)amino)-4-oxobutanamido)ethane-1-sulfonate

To a solution of compound 86 (52.94 mg, 123 μmol) in DCM (5 mL) were added EDAC (26.0 μL, 147.6 μmol), NHS (14.75 mg, 123 μmol) and DMF (0.5 mL). The reaction mixture was stirred for 4 h at RT then was added EDC (10 μL, 45.7 μmol) and stirring was carried on at RT overnight. The reaction medium was stored at −20° C. and used such as for the synthesis of compound 88.

### Compound 88: sodium (20S,23S)-28-(2-allyloxy-2-carboxyethyl)-20-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)-23-isopropyl-14,22,25,29,32-pentaoxo-2,5,8,11-tetraoxa-15,21,24,28,33-pentaazapentatriacontane-35-sulfonate

To a solution of compound 62 (50 mg, 43.7 μmol) in DCM (4 mL) was added, dropwise under Ar, the reaction medium containing compound 87 (23.06 mg, 43.7 μmol). The reaction medium was stirred at RT for 15 min, concentrated in vacuo and purified by flash chromatography on 15 g of silica gel (gradient elution DMC/MeOH/H₂O) to give 26 mg of compound 88 as a white lacquer (38%).

### Example 42: sodium (20S,23S)-28-(2-carboxyethyl)-20-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)-23-isopropyl-14,22,25,29,32-pentaoxo-2,5,8,11-tetraoxa-15,21,24,28,33-pentaazapentatriacontane-35-sulfonate

To a solution of compound 88 (14 mg, 9 μmol) in THF were added dimedone (2.57 mg, 18.0 μmol) and tetrakis(triphenylphosphine)palladium (0) (5.3 mg, 4.5 μmol). The reaction medium was stirred for 1 h at RT then were added dimedone (2.57 mg, 18.0 μmol) and tetrakis(triphenyl-phosphine)palladium (0) (5.3 mg, 4.5 μmol) and stirring was carried on at RT overnight. At that time, were added dimedone (2.57 mg, 18.0 μmol) and tetrakis(triphenyl-phosphine)palladium (0) (5.3 mg, 4.5 μmol). The reaction medium was stirred for 4 h at RT, filtered, concentrated in vacuo and purified by flash chromatography on 4 g of silica gel (gradient elution DCM/MeOH/H₂O) to give 7 mg of example 42 as a white lacquer (51%).

RMN ¹H (500 MHz, δ in ppm, DMSO-d6): 50:50 conformer mixture; 0.78 (d, J=6.8 Hz, 6H); 0.81 (d, J=6.8 Hz, 6H); 0.86 (m, 6H); 1.00 (s, 3H); 1.04 (d, J=7.1 Hz, 3H); 1.11 (s, 3H); 1.15 to 1.41 (m, 5H); 1.53 to 1.70 (m, 4H); 1.80 (m, 1H); 2.00 (m, 1H); 2.11 to 2.58 (partially masked m, 13H); 2.70 (m, 2H); 2.92 to 3.05 (m, 5H); 3.23 (s, 3H); 3.25 to 3.58 (partially masked m, 23H); 3.81 (s, 3H); 3.88 (d, J=2.0 Hz, 1H); 4.10 to 4.32 (m, 5H); 4.91 (dd, J=3.8 and 9.9 Hz, 1H); 5.10 (m, 1H); 5.80 (d, J=15.2 Hz, 1H); 6.47 (ddd, J=3.8, 11.4 and 15.2 Hz, 1H); 7.06 (d, J=8.6 Hz, 1H); 7.18 (dd, J=2.4 and 8.6 Hz, 1H); 7.20 to 7.32 (m, 6H); 7.67 to 7.93 (m, 3H); 8.18 to 8.60 (m, 4H). LCMS (E): ES m/z=747.5 [M+2H]²⁺; m/z=1491 [M−H]⁻; m/z=1493 [M+H]⁺; t_{R}=4.74 min.

### Synthesis of Example 43: sulfo-Val-GlucoseGln-C52 benzylic amine

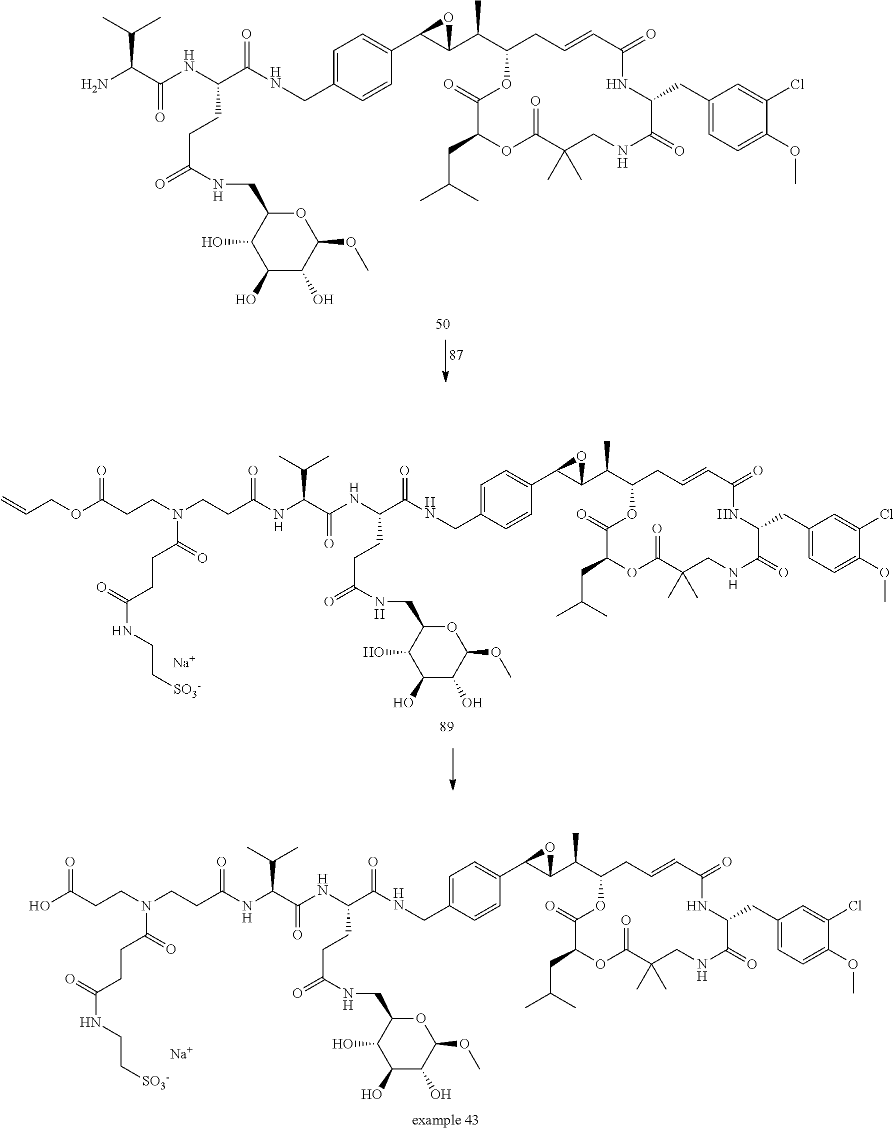

Compound 89: sodium (6S,9S)-14-(3-(allyloxy)-3-oxopropyl)-6-((4-((2R,3R)-3-((S)-1-((3S,10R, 16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)-9-isopropyl-3,8,11,15,18-pentaoxo-1-((2R,3S,4S,5R,6R)-3,4,5-trihydroxy-6-methoxytetrahydro-2H-pyran-2-yl)-2,7,10,14, 19-pentaazahenicosane-21-sulfonate

To a solution of compound 50 (55 mg, 49.9 μmol) in DMF (3 mL) was added a a solution of compound 87 in DMF (1.66 mL at 30 mM, 49.9 μmol). The reaction medium was stirred for 18 h at RT, filtered over 0.45 μm, washed twice with DMF (0.8 mL) and purified by reverse phase chromatography on a 5 μm C18 column 30×150 mm (gradient elution MeCN/0.2N ammonium acetate pH 5.6) to give 19.8 mg of compound 89 as a white solid (26%).

RMN ¹H (500 MHz, δ in ppm, DMSO-d6): 60:40 conformer mixture; 0.78 (d, J=6.8 Hz, 6H); 0.81 (d, J=6.8 Hz, 6H); 0.81 to 0.87 (m, 6H); 0.99 (s, 3H); 1.03 (d, J=7.1 Hz, 3H); 1.12 (s, 3H); 1.30 (m, 1H); 1.52 to 1.62 (m, 2H); 1.80 (m, 3H); 1.85 to 2.00 (m, 3H); 2.10 to 2.30 (m, 5H); 2.35 to 2.48 (m, 2H); 2.53 (m, 6H); 2.68 (m, 2H); 2.88 to 3.15 (m, 9H); 3.28 (m, 3H); 3.38 (m, 3H); 3.40 to 3.58 (m, 5H); 3.80 (s, 3H); 3.87 (d, J=2.3 Hz, 1H); 4.01 (d, J=8.0 Hz, 1H); 4.10 to 4.24 (m, 6H); 4.55 (m, 1.2H); 4.57 (0.8H); 4.91 (dd, J=3.8 et 9.9 Hz, 1H); 4.95 (d, J=5.0 Hz, 1H); 5.01 (d, J=5.0 Hz, 1H); 5.06 (d, J=5.0 Hz, 1H); 5.10 (m, 1H); 5.18 to 5.35 (m, 2H); 5.80 (dd, J=2.0 and 15.2 Hz, 1H); 5.92 (m, 1H); 6.47 (ddd, J=3.8, 11.4 and 15.2 Hz, 1H); 7.06 (d, J=8.6 Hz, 1H); 7.18 (dd, J=2.4 and 8.6 Hz, 1H); 7.23 (m, 5H); 7.29 (d, J=2.4 Hz, 1H); 7.69 (t, J=6.8 Hz, 1H); 7.88 (t, J=6.8 Hz, 1H); 7.98 (d, J=9.0 Hz, 0.4 H); 8.04 (d, J=7.0 Hz, 0.4H); 8.08 (d, J=9.0 Hz, 0.6H); 8.14 (d, J=7.0 Hz, 0.6H); 8.32 (t, J=7.0 Hz, 1H); 8.38 (d, J=8.0 Hz, 1H). LCMS (A): ES m/z=746.5 [M+2H]²⁺; m/z=1490 [M−H]⁻; m/z=1492 [M+H]⁺; t_{R}=1.4 min.

### Example 43: sodium (6S,9S)-14-(2-carboxyethyl)-6-((4-((2R,3R)-3-((S)-1-((3S,10R,16S,E)-10-(3-chloro-4-methoxybenzyl)-3-isobutyl-6,6-dimethyl-2,5,9,12-tetraoxo-1,4-dioxa-8,11-diazacyclohexadec-13-en-16-yl)ethyl)oxiran-2-yl)benzyl)carbamoyl)-9-isopropyl-3,8,11,15,18-pentaoxo-1-((2R,3S,4S,5R,6R)-3,4,5-trihydroxy-6-methoxytetrahydro-2H-pyran-2-yl)-2,7,10,14,19-pentaazahenicosane-21-sulfonate

To a solution of compound 89 (19 mg, 12.6 μmol) in DMF (1 mL) were added barbituric acid (5.94 mg, 37.7 μmol) and a solution of tetrakis (triphenylphosphine)palladium(0) (0.74 mg, 0.63 μmol) in DMF (0.5 mL). The reaction medium was stirred for 3 h at RT then added barbituric acid (5.94 mg, 37.7 μmol) and a solution of tetrakis(triphenylphosphine)palladium(0) (0.74 mg, 0.63 μmol) in DMF (0.5 mL). The reaction medium was stirred at RT overnight, diluted with DMF, filtered over 0.45 μm and purified by two consecutive reverse phase chromatographies on a 5 μm C18 column 30×150 mm (gradient elution MeCN/0.2N ammonium acetate pH 5.6) to give 4.7 mg of example 43 as a white solid (25%).

RMN ¹H (500 MHz, δ in ppm, DMSO-d6): 0.80 (m, 6H); 0.85 (m, 6H); 0.99 (s, 3H); 1.03 (d, J=7.3 Hz, 3H); 1.11 (s, 3H); 1.33 (m, 1H); 1.58 (m, 2H); 1.78 (m, 2H); 1.90 (m, 2H); 2.03 to 2.55 (partially masked m, 11H); 2.70 (m, 2H); 2.90 to 3.58 (partially masked m, 19H); 3.18 (s, 3H); 3.81 (s, 3H); 3.88 (s, 1H); 4.00 to 4.40 (m, 8H); 4.90 (m, 1H); 4.99 (m, 1H); 5.80 (d, J=15.2 Hz, 1H); 6.46 (ddd, J=3.8, 11.4 and 15.2 Hz, 1H); 7.05 (d, J=8.6 Hz, 1H); 7.18 (dd, J=2.4 and 8.6 Hz, 1H); 7.22 (m, 4H); 7.29 (d, J=2.4 Hz, 1H); 7.31 (m, 1H); 8.30 to 8.70 (m, 5H). LCMS (A): ES m/z=1448 [M−H]⁻; m/z=1450 [M+H]⁺; t_{R}=1.58 min.

For illustrative purposes, two conjugates derived from WO2011/001052, ADC1 and ADC2 depicted below, and one conjugate described in PCT/EP2016/076603, ADC3 depicted below, were also tested.

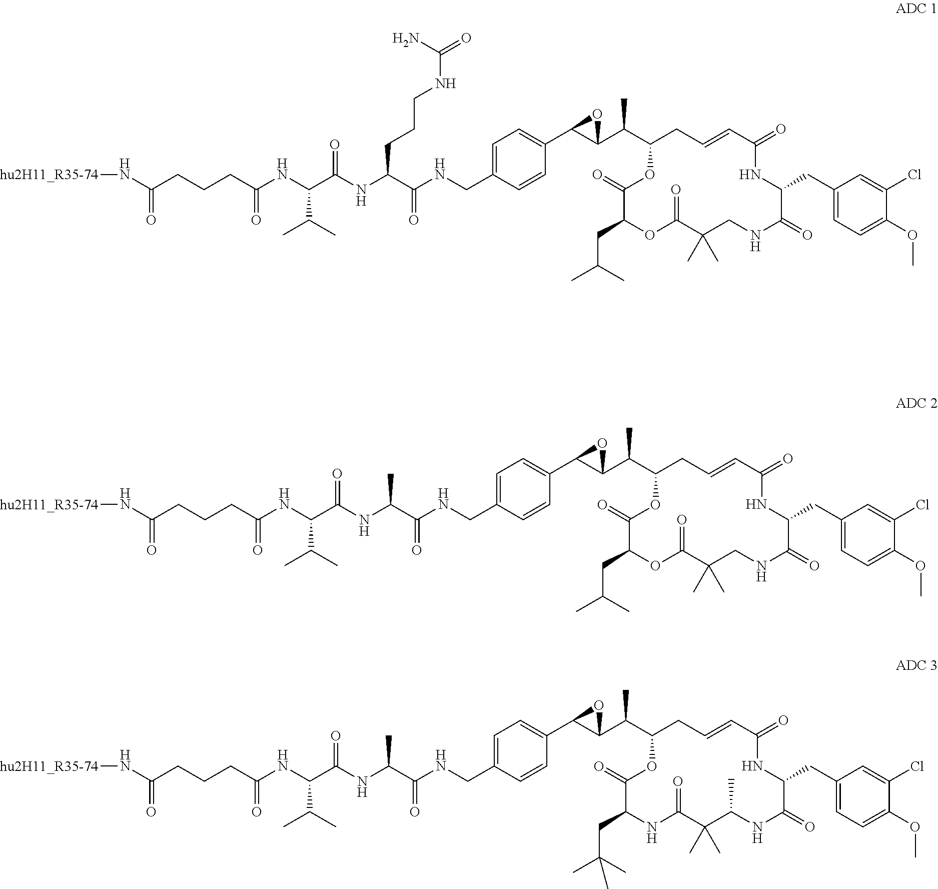

### Physico-Chemical Results

The conjugates of formula (V) were subjected to stability studies evaluating aggregation (looking at monomeric purity) and loss of DAR upon removal of the organic co-solvent used in the formulation and under accelerated stress conditions (10 day heating at 40° C.).

### Stability in the Absence of NMP

ADC stored in 10 mM phosphate 140 mM NaCl pH6.5 5% NMP were filtered on a SEC column to remove the NMP, formulated in 10 mM phosphate 70 mM NaCl 5% sucrose pH6.5 and analyzed by HRMS and SEC HPLC to determine the DAR and the monomeric purity.

TABLE I

| | 5% NMP | | no NMP | | ΔDAR | Δ % monomer | protein |
|---|---|---|---|---|---|---|---|
| | DAR | % monomer | DAR | % monomer | (%) | (%) | recovery |
| ADC1 | 4.1 | 99 | 3.25 | 88.7 | −21% | −10% | quant. |
| ADC3 | 3.5 | 99.7 | 3.5 | 99.5 | 0% | −0.2% | n.d. |
| Ex.3 | 3.4 | 99.8 | 3.2 | 99.7 | −6% | −0.1% | 88% |
| Ex.6 | 4.05 | 99 | 3.4 | 99 | −16% | 0% | 75% |
| Ex.16* | 2.5 | 99.8 | 2.3 | 99.8 | −8% | 0% | 73% |
| Ex.19 | 2.8 | 97.9 | 2.4 | 97.8 | −14% | −0.1% | 72% |
| Ex.23* | 2.9 | 100 | 2.4 | 100 | −17% | 0% | 76% |
| Ex.26 | 3.9 | 100 | 3.9 | 100 | 0% | 0% | n.d. |
| Ex.29 | 4 | 98.4 | 3.9 | 100 | −2.5% | 0% | 76% |
| Ex.32 | 2.8 | 95.1 | 2.9 | 94.1 | 0 | −11% | 82% |

*After 10 months at 4° C., significant aggregation was observerd for Ex.16 and Ex.23 (respectively −22% and −19% in terms of monomeric purity), therefore both batches were purified by gel filtration on Superdex 200 pg prior to performing stability studies.

Stability after 10 Days at 40° C. in Phosphate Buffer

ADC in 10 mM phosphate 70 mM NaCl 5% sucrose pH6.5 were heated at 40° C. during 10 days and analyzed by HRMS and SEC HPLC to determine the DAR and the monomeric purity.

TABLE II

| | At t0 | | After 10 d at 40° C. | | ΔDAR | Δ % monomer | protein |
|---|---|---|---|---|---|---|---|
| | DAR | % monomer | DAR | % monomer | (%) | (%) | recovery |
| ADC1 | 3.25 | 88.7 | 3.0 | 85.7% | −8% | −3% | 56% |
| ADC3 | 3.5 | 99.5 | 3 | 85 | −14% | −15% | 46% |
| Ex.3 | 3.2 | 99.7 | 2.9 | 92 | −9% | −8% | 96% |
| Ex.6 | 3.4 | 99 | 3.3 | 97 | −3% | −2% | 91% |
| Ex.16 | 2.3 | 99.8 | 1.8 | 93.8 | −22% | −6% | 89% |
| Ex.19 | 2.4 | 97.8 | 1.8 | 92.9 | −25% | −5% | quant. |
| Ex.23 | 2.4 | 100 | 2.4 | 91.6 | 0% | −8% | 91% |
| Ex.26 | 3.9 | 100 | 3.6 | 100 | −8% | 0% | n.d. |
| Ex.29 | 3.9 | 100 | 3.9 | 98.7 | 0% | −1% | 97% |
| Ex.32 | 2.9 | 94.1 | 2.7 | 89.8 | −7% | −5% | 95% |
| Ex.41 | 3.3 | 97.9 | 3.05 | 75.3 | −7.5% | −23% | quant. |

Evaluation of Drug Release after Treatment of the Conjugates of Formula (V) by Cathepsin B

Drug release by Cathepsin B was evaluated in vitro by incubating the ADO (1.5 μg/mL) in the presence of human Cathepsin B (Calbiochem #219362, 40 μg/mL) in acetate buffer pH 5 at 37° C. for 24 h and detecting the drug by UPLC-MS-SIR-DAD-ELSD. The slope was determined based on the kinetics over the 1^{st} hour, the half-life on the 24 h kinetics.

TABLE III

| | slope | t_{1/2} (h) |
|---|---|---|
| ADC1 | 34.3 | 2 |
| ADC2 | 14.1 | 15 |
| Ex.16 | 12.8 | >24 |
| Ex.19 | 22.3 | 3.5 |
| Ex.23 | 19.3 | 6 |
| Ex.32 | 33.5 | 2 |

Cryptophycin conjugates of formula (V) were cleaved by human Cathepsin B with a kinetics similar to the one of reference ADCs.

### Pharmacological Results

The conjugates of formula (V) were subjected to pharmacological tests for determining their antitumoral effect.

### Evaluation of the Inhibition of Proliferation of the MDA-MB-231 Cell Line by the Conjugates of Formula (V)

MDA-MB-231 cells in their exponential growth phase were trypsinized and resuspended in their culture medium (DMEM/F12 Gibco #21331, 10% FCS Gibco #10500-056, 2 nM glutamine Gibco #25030). The cell suspension was seeded in Cytostar 96-well culture plates (GE Healthcare Europe, #RPNQ0163) in the whole culture medium containing serum at a density of 5000 cells/well. After incubation for 4 h, successive dilutions of the ADC were added to the wells at concentrations decreasing from 10-7 to 10⁻¹² M (in triplicate for each concentration). The cells were cultured at 37° C. in an atmosphere containing 5% CO₂ in the presence of the ADC for 3 d. On the 4^{th} day, 10 μL of a ¹⁴C-thymidine solution (0.1 μCi/well, Perkin Elmer #NEC56825000) were added to each well. The incorporation of ¹⁴C-thymidine was measured 96 h after the start of the experiment with a microbeta radioactivity counter (Perkin Elmer). The data were expressed in the form of a percentage of survival by determining the ratio between the reduced count obtained with the cells treated with the ADC and the count obtained with the cells of the control wells (treated with the culture medium alone). In certain experiments, the naked antibody was added to the wells at a concentration of 1 μM at the start of the experiment and the inhibition of proliferation was measured as described previously.

TABLE IV

| | IC₅₀ (pM), MDA-MB-231 | | |
|---|---|---|---|
| | | in the presence of | Selectivity |
| | ADC alone | naked antibody | ratio |
| ADC1 | 42 | 4571 | 109 |
| ADC2 | 46 | 9377 | 204 |
| Ex.3 | 19 | 5602 | 295 |
| Ex.6 | 22 | 7417 | 337 |
| Ex.16 | 39 | 13908 | 357 |
| Ex.19 | 29 | 18243 | 629 |
| Ex.23 | 37 | 9022 | 243 |
| Ex.26 | 27 | 33689 | 1248 |
| Ex.29 | 67 | >100000 | >1493 |
| Ex.32 | 25 | 7953 | 318 |
| Ex.35 | 25 | 11272 | 451 |
| Ex.41 | 34 | 5043 | 148 |

Cryptophycin conjugates of formula (V), as well as ADC1 and ADC2, were found to inhibit the proliferation of MDA-MB-231 cell line with IC₅₀ ranging from 19 μM to 67 μM and selectivity ratio ADC alone vs ADC+naked antibody between 109 and >1493.

Determination of the MTD of the Conjugates of Formula (V) Following Single i.v. Administration in SCID Mice

MTD was determined as the maximal dose that does not induce 15% body weight loss during 3 consecutive days for an individual mouse or 20% body weight loss during 1 day or mortality. It was evaluated after a single intravenous (i.v.) bolus injection in 3 female SCID mice and during a period of 28 days post-treatment.

TABLE V

| | MTD (mg/kg) |
|---|---|
| ADC1 | 20 |
| ADC2 | 30 |
| Ex.3 | 30 |
| Ex.6 | >40 |
| Ex.16 | n.d. |
| Ex.19 | n.d. |
| Ex.23 | n.d. |
| Ex.26 | 30 |
| Ex.29 | 40 |
| Ex.32 | n.d. |
| Ex.35 | n.d. |
| Ex.41 | 30 |

Tested cryptophycin conjugates of formula (V) displayed MTD in SCID mice ranging from 30 mg/kg to >40 mg/kg.

Evaluation of the In Vivo Antitumor Activity of Conjugates of Formula (V) Against MDA-MB-231 in SCID Mice Following Single i.v. Administration

In vivo antitumor activity was evaluated at 3 dose-levels against measurable breast MDA-MB-231 xenografts implanted s.c. in female SCID mice. Control groups were left untreated. Conjugates were administered by a single i.v. bolus injection, the day of the treatment was indicated on each graph by an arrow (▾).

For the evaluation of antitumor activity of conjugates, animals were weighed twice weekly and tumors were measured twice weekly by caliper. Animal body weights included the tumor weights. Tumor volume were calculated using the formula mass (mm³)=[length (mm)×width (mm)²]/2. The primary efficacy end points were ΔT/ΔC, percent median regression, partial and complete regressions (PR and CR). Changes in tumor volume for each treated (T) and control (C) were calculated for each tumor by subtracting the tumor volume on the day of first treatment (staging day) from the tumor volume on the specified observation day. The median ΔT was calculated for the treated group and the median ΔC was calculated for the control group. Then the ratio ΔT/ΔC was calculated and expressed as a percentage: ΔT/ΔC=(delta T/delta C)×100.

The percentage of tumor regression was defined as the % of tumor volume decrease in the treated group at a specified observation day (t) compared to its volume on the first day of first treatment (t0). At a specific time point and for each animal, % regression was calculated. The median % regression was then calculated for the group. % regression (at t)=((Volumeₜ₀−Volumeₜ)/Volumeₜ₀)×100. Regressions were defined as partial (PR) if the tumor volume decreased to 50% of the tumor volume at the start of treatment and complete (CR) when tumor volume cannot be measured (0 mm³). Tumor free survivor (TFS) was defined as the animals with undetectable tumors at the end of the study (>100 days post last treatment).

Evaluation of the In Vivo Antitumor Activity of Ex. 6 Against MDA-MB-231 in SCID Mice Following Single i.v. Administration

TABLE VI

| | | Median | | | | |
|---|---|---|---|---|---|---|
| | | ΔT/ΔC | Median | | | |
| | Dose | in % | % of | Regressions | | |
| | (mg/kg) | (D36) | regression | PR | CR | TFS |
| Control | — | — | — | — | — | — |
| ADC2 | 2.5 | 8 | — | 1 | 1 | 1 |
| Ex.6 | 5 | <0 | 100% | 5 | 4 | — |
| | 2.5 | <0 | 4% | 2 | 1 | — |
| | 1.25 | 33 | — | — | — | — |

Evaluation of the In Vivo Antitumor Activity of Ex. 16, Ex. 19, Ex. 23 and Ex. 32 Against MDA-MB-231 in SCID Mice Following Single i.v. Administration at 2.5 Mg/Kg

TABLE VII

| | | Median | | | | |
|---|---|---|---|---|---|---|
| | | ΔT/ΔC | Median | | | |
| | Dose | in % | % of | Regressions | | |
| | (mg/kg) | (D59) | regression | PR | CR | TFS |
| Control | — | — | — | — | — | — |
| ADC2 | 2.5 | <0 | 14% | — | — | — |
| Ex.16 | 2.5 | <0 | 92% | 6 | 3 | — |
| Ex.19 | 2.5 | <0 | 100% | 6 | 5 | 2 |
| Ex.23 | 2.5 | <0 | 100% | 6 | 6 | 1 |
| Ex.32 | 2.5 | <0 | 100% | 6 | 6 | 2 |

Evaluation of the In Vivo Antitumor Activity of Ex. 16, Ex. 19, Ex. 23 and Ex. 32 Against MDA-MB-231 in SCID Mice Following Single i.v. Administration at 1.25 Mg/Kg

TABLE VIII

| | | Median | | | | |
|---|---|---|---|---|---|---|
| | | ΔT/ΔC | Median | | | |
| | Dose | in % | % of | Regressions | | |
| | (mg/kg) | (D50) | regression | PR | CR | TFS |
| Control | — | — | — | — | — | — |
| ADC2 | 1.25 | 14 | — | — | — | — |
| Ex.16 | 1.25 | 7 | — | — | — | — |
| Ex.19 | 1.25 | 1 | — | 1 | 1 | — |
| Ex.23 | 1.25 | 0 | — | 1 | — | — |
| Ex.32 | 1.25 | 9 | — | 2 | 1 | — |

Evaluation of the In Vivo Antitumor Activity of Ex. 26 Against MDA-MB-231 in SCID Mice Following Single i.v. Administration

TABLE IX

| | | Median | | | | |
|---|---|---|---|---|---|---|
| | | ΔT/ΔC | Median | | | |
| | Dose | in % | % of | Regressions | | |
| | (mg/kg) | (D36) | regression | PR | CR | TFS |
| Control | — | — | — | — | — | — |
| ADC2 | 2.5 | 8 | — | 1 | — | 1 |
| Ex.26 | 5 | <0 | 100% | 6 | 6 | 2 |
| | 2.5 | <0 | 100% | 6 | 4 | 1 |
| | 1.25 | 10 | — | 1 | — | — |

Evaluation of the In Vivo Antitumor Activity of Ex. 29 Against MDA-MB-231 in SCID Mice Following Single i.v. Administration

TABLE X

| | | Median | | | | |
|---|---|---|---|---|---|---|
| | | ΔT/ΔC | Median | | | |
| | Dose | in % | % of | Regressions | | |
| | (mg/kg) | (D27) | regression | PR | CR | TFS |
| Control | — | — | — | — | — | — |
| Ex.29 | 4 | <0 | 100% | 6 | 6 | 6 |
| | 2 | <0 | 100% | 6 | 6 | 4 |
| | 1 | 0 | — | — | — | — |

Evaluation of the In Vivo Antitumor Activity of Ex. 35 Against MDA-MB-231 in SCID Mice Following Single i.v. Administration

TABLE XI

| | | Median | | | | |
|---|---|---|---|---|---|---|
| | | ΔT/ΔC | Median | | | |
| | Dose | in % | % of | Regressions | | |
| | (mg/kg) | (D27) | regression | PR | CR | TFS |
| Control | — | — | — | — | — | — |
| Ex.35 | 4 | <0 | 100% | 6 | 6 | 4 |
| | 2 | <0 | 100% | 6 | 4 | 1 |
| | 1 | 3 | — | — | — | — |

Evaluation of the In Vivo Antitumor Activity of Ex. 41 Against MDA-MB-231 in SCID Mice Following Single i.v. Administration

TABLE XII

| | | Median | | | | |
|---|---|---|---|---|---|---|
| | | ΔT/ΔC | Median | | | |
| | Dose | in % | % of | Regressions | | |
| | (mg/kg) | (D49) | regression | PR | CR | TFS |
| Control | — | — | — | — | — | — |
| Ex.26 | 4 | <0 | 100% | 6 | 6 | 6 |
| | 2 | <0 | 100% | 6 | 6 | 4 |
| | 1 | <0 | 51% | 3 | 2 | 1 |
| Ex.41 | 4 | <0 | 100% | 6 | 4 | 2 |
| | 2 | <0 | 25% | 2 | — | — |
| | | (D46) | | | | |
| | 1 | 20 | — | — | — | — |

All tested cryptophycin ADC of the invention, as well as ADC2, displayed antitumor activity at doses ranging from 1 mg/kg to 5 mg/kg.

It is therefore apparent that the compounds (V) of the invention have an anticancer activity.

Accordingly, in another of its aspects, the invention also relates to the use of cryptophycin conjugates of formula (V) as anticancer agents.

Accordingly, in another of its aspects, the invention also relates to the use of cryptophycin conjugates of formula (V) for the preparation of a medicament for treating cancer, for instance breast cancer.

The present invention, according to another of its aspects, also provides conjugates of formula (V) according to the present invention for use in the treatment of cancer.

The present invention, according to another of its aspects, also provides medicaments which comprise at least one conjugate of formula (V).

These medicaments are employed therapeutically, especially in the treatment of cancer, for instance breast cancer.

According to another of its aspects, the present invention relates to pharmaceutical compositions comprising as active principle a conjugate of formula (V) according to the invention. These pharmaceutical compositions comprise an effective dose of at least one conjugate of formula (V) according to the invention and also at least one pharmaceutically acceptable excipient.

Thus, according to another aspect, the present invention relates to a pharmaceutical composition comprising at least one conjugate of formula (V) according to the present invention, and also at least one pharmaceutically acceptable excipient.

The said excipients are selected, in accordance with the pharmaceutical form and method of administration desired, from the customary excipients, which are known to a person skilled in the art.

The present invention, according to another of its aspects, also provides a method of treating the pathologies indicated above such as cancer, for instance breast cancer, which comprises administering to a patient an effective dose of a conjugate of formula (V) according to the invention.

1-22. (canceled)

23. A compound of formula (IV): 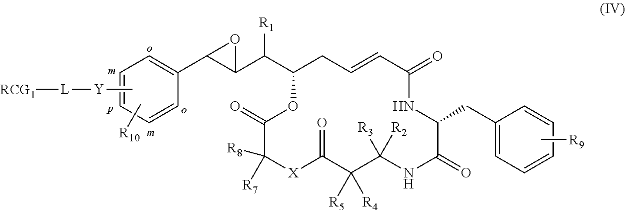
wherein:
R₁ is (C₁-C₆)alkyl;
R₂ and R₃ are, independently of each other, hydrogen or (C₁-C₆)alkyl;
or alternatively R₂ and R₃ form, together with the carbon atom to which they are attached, a (C₃-C₆)cycloalkyl or a (C₃-C₆)heterocycloalkyl group;
R₄ and R₅ are, independently of each other, hydrogen, (C₁-C₆)alkyl, (C₁-C₆)alkylene-C(O)OH, (C₁-C₆)alkylene-NHR₁₂, (C₁-C₆)alkylene-OH, or (C₁-C₆)alkylene-SH;
or alternatively R₄ and R₅ form, together with the carbon atom to which they are attached, a (C₃-C₆)cycloalkyl or a (C₃-C₆)heterocycloalkyl group;
X is O or N(R₆);
R₆ is hydrogen or (C₁-C₆)alkyl;
R₇ and R₈ are, independently of each other, hydrogen, (C₁-C₆)alkyl, (C₁-C₆)alkylene-C(O)OH, or (C₁-C₆)alkylene-N[(C₁-C₆)alkyl]₂;
or alternatively R₇ and R₈ form, together with the carbon atom to which they are attached, a (C₃-C₆)cycloalkyl group or a (C₃-C₆)heterocycloalkyl group;
each R₉ is independently hydrogen, halogen, NH₂, NH(C₁-C₆)alkyl, N[(C₁-C₆)alkyl]₂, NH(C₃-C₆)cycloalkyl, OH, O(C₁-C₄)alkyl, or (C₃-C₆)heterocycloalkyl;
each R₁₀ is independently hydrogen or (C₁-C₄)alkyl;
Y is NR₁₁—(C₁-C₆)alkyl-, —O—(C₁-C₆)alkyl-, or —S—(C₁-C₆)alkyl-;
R₁₁ and R₁₂ are, independently of each other, hydrogen or (C₁-C₆)alkyl;
L represents a linker of formula (II): 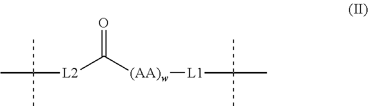
wherein:
L1 is of formula (III): 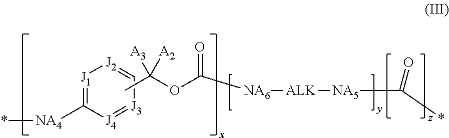
wherein:
(i) x is 0, y is 0, and z is 0, or
(ii) x is 1, y is 0, and z is 0, or
(iii) x is 1, y is 1, and z is 1, or
(iv) x is 1, y is 1, and z is 0, or
(v) x is 0, y is 1, and z is 0;
J₁, J₂, J₃ and J₄ are, independently of each other, CA₁ or N;
ALK is (C₁-C₁₂)alkylene;
A₁, A₂, A₃, A₄, A₅, and A₆ are, independently of each other, hydrogen or (C₁-C₆)alkyl, wherein L1 is bonded directly to —NR₁₁—, —O—, or —S— of Y;
(AA)w is a sequence of w substituted (AAₛ) or non-substituted amino acids (AAₙₛ) connected together via peptide bonds;
w is 1, 2, 3, 4, 5, 6, 7, 8, 9, 10, 11, or 12;
wherein:
if (AA)w contains at least one substituted amino acid (AAₛ), then L2 is a single bond, a (C₁-C₆)alkyl group, a (C₁-C₆)alkyl-(OCH₂CH₂)ᵢ group, a (C₁-C₆)alkyl-(OCH₂CH₂)ᵢ—O(C₁-C₆)alkyl group, a (CH₂CH₂O)ᵢ(C₁-C₆)alkyl group, a CH(SO₃H)—(C₁-C₆)alkyl group, a (C₁-C₆)alkyl-CH(SO₃H) group, a (C₁-C₆)alkyl-cyclohexyl group, a C(O)—(C₁-C₆)alkyl group, a C(O)—(C₁-C₆)alkyl-(OCH₂CH₂)ᵢ group, a C(O)—(C₁-C₆)alkyl-(OCH₂CH₂)ᵢ—O(C₁-C₆)alkyl group, a C(O)—(CH₂CH₂O)ᵢ(C₁-C₆)alkyl group, a C(O)—CH(SO₃H)—(C₁-C₆)alkyl group, a C(O)—(C₁-C₆)alkyl-CH(SO₃H) group, a C(O)—(C₁-C₆)alkyl-cyclohexyl group, a NA₈-(C₁-C₆)alkyl group, a NA₈-(C₁-C₆)alkyl-(OCH₂CH₂)ᵢ group, a NA₈-(C₁-C₆)alkyl-(OCH₂CH₂)ᵢ—O(C₁-C₆)alkyl group, a NA₈-(CH₂CH₂O)ᵢ(C₁-C₆)alkyl group, a NA₈-(C₁-C₆)alkyl-CH(SO₃H) group, a C(O)—NA₈-(C₁-C₆)alkyl group, a C(O)—NA₈-(C₁-C₆)alkyl-(OCH₂CH₂)ᵢ group, a C(O)—NA₈-(C₁-C₆)alkyl-(OCH₂CH₂)ᵢ—O(C₁-C₆)alkyl group, a C(O)—NA₈-(CH₂CH₂O)ᵢ(C₁-C₆)alkyl group, a C(O)—NA₈-(C₁-C₆)alkyl-CH(SO₃H) group, a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(O)—(C₁-C₆)alkyl group, a (C₁-C₆)alkyl-NA₇C(O)—(C₁-C₆)alkyl-(OCH₂CH₂)ᵢ group, a (C₁-C₆)alkyl-C(O)NA₇-(C₁-C₆)alkyl group, a (C₁-C₆)alkyl-C(O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(O)—NA₇-(C₁-C₆)alkyl group, a C(O)—(C₁-C₆)alkyl-NA₇ group, a C(O)—NA₇-(CH₂CH₂O)ᵢ(C₁-C₆)alkyl group, a C(O)—NA₇-aryl group, a C(O)—NA₇-heteroaryl group, a C(O)—(C₁-C₆)alkyl-NA₇C(O)—(C₁-C₆)alkyl group, a C(O)—(C₁-C₆)alkyl-NA₇C(O)—(C₁-C₆)alkyl-(OCH₂CH₂)ᵢ group, a C(O)—(C₁-C₆)alkyl-C(O)NA₇-(C₁-C₆)alkyl group, a C(O)—(C₁-C₆)alkyl-C(O)NA₇-(C₁-C₆)alkyl-(OCH₂CH₂)ᵢ group, a C(O)—(C₁-C₆)alkyl-NA₇-(C₁-C₆)alkyl group, a C(O)—(C₁-C₆)alkyl-NA₇-(C₁-C₆)alkyl-(OCH₂CH₂)ᵢ group, a C(O)—(C₁-C₆)alkyl-(OCH₂CH₂)ᵢ—NA₇-(C₁-C₆)alkyl group, a NA₈-(C₁-C₆)alkyl-NA₇ group, a NA₈-(C₁-C₆)alkyl-NA₇C(O)—(C₁-C₆)alkyl group, a NA₈-(C₁-C₆)alkyl-NA₇C(O)—(C₁-C₆)alkyl-(OCH₂CH₂)ᵢ group, a NA₈-(C₁-C₆)alkyl-C(O)NA₇-(C₁-C₆)alkyl group, a NA₈-(C₁-C₆)alkyl-C(O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ—NA₇-(C₁-C₆)alkyl group, a C(O)—NA₈-(C₁-C₆)alkyl-NA₇ group, a C(O)—NA₈-(C₁-C₆)alkyl-NA₇C(O)—(C₁-C₆)alkyl group, a C(O)—NA₈-(C₁-C₆)alkyl-NA₇C(O)—(C₁-C₆)alkyl-(OCH₂CH₂)ᵢ group, a C(O)—NA₈-(C₁-C₆)alkyl-C(O)NA₇-(C₁-C₆)alkyl group, a C(O)—NA₈-(C₁-C₆)alkyl-C(O)NA₇-(C₁-C₆)alkyl-(OCH₂CH₂)ᵢ group, a C(O)—NA₈-(C₁-C₆)alkyl-NA₇-(C₁-C₆)alkyl group, a C(O)—NA₈-(C₁-C₆)alkyl-NA₇-(C₁-C₆)alkyl-(OCH₂CH₂)ᵢ group, or a C(O)—NA₈-(C₁-C₆)alkyl-(OCH₂CH₂)ᵢ—NA₇-(C₁-C₆)alkyl group;
if (AA)w represents a sequence of w non-substituted amino acids (AAₙₛ), then L2 is a NA₇-(C₁-C₆)alkyl group, a (C₁-C₆)alkyl-NA₇ group, a NA₇-(CH₂CH₂O)ᵢ(C₁-C₆)alkyl group, a NA₇-aryl group, a NA₇-heteroaryl group, a (C₁-C₆)alkyl-NA₇C(O)—(C₁-C₆)alkyl group, a (C₁-C₆)alkyl-NA₇C(O)—(C₁-C₆)alkyl-(OCH₂CH₂)ᵢ group, a (C₁-C₆)alkyl-C(O)NA₇-(C₁-C₆)alkyl group, a (C₁-C₆)alkyl-C(O)NA₇-(C₁-C₆)alkyl-(OCH₂CH₂)ᵢ group, a (C₁-C₆)alkyl-NA₇-(C₁-C₆)alkyl group, a (C₁-C₆)alkyl-NA₇-(C₁-C₆)alkyl-(OCH₂CH₂)ᵢ group, a (C₁-C₆)alkyl-(OCH₂CH₂)ᵢ—NA₇-(C₁-C₆)alkyl group, a C(O)—NA₇-(C₁-C₆)alkyl group, a C(O)—(C₁-C₆)alkyl-NA₇ group, a C(O)—NA₇-(CH₂CH₂O)ᵢ(C₁-C₆)alkyl group, a C(O)—NA₇-aryl group, a C(O)—NA₇-heteroaryl group, a C(O)—(C₁-C₆)alkyl-NA₇C(O)—(C₁-C₆)alkyl group, a C(O)—(C₁-C₆)alkyl-NA₇C(O)—(C₁-C₆)alkyl-(OCH₂CH₂)ᵢ group, a C(O)—(C₁-C₆)alkyl-C(O)NA₇-(C₁-C₆)alkyl group, a C(O)—(C₁-C₆)alkyl-C(O)NA₇-(C₁-C₆)alkyl-(OCH₂CH₂)ᵢ group, a C(O)—(C₁-C₆)alkyl-NA₇-(C₁-C₆)alkyl group, a C(O)—(C₁-C₆)alkyl-NA₇-(C₁-C₆)alkyl-(OCH₂CH₂)ᵢ group, a C(O)—(C₁-C₆)alkyl-(OCH₂CH₂)ᵢ—NA₇-(C₁-C₆)alkyl group, a NA₈-(C₁-C₆)alkyl-NA₇ group, a NA₈-(C₁-C₆)alkyl-NA₇C(O)—(C₁-C₆)alkyl group, a NA₈-(C₁-C₆)alkyl-NA₇C(O)—(C₁-C₆)alkyl-(OCH₂CH₂)ᵢ group, a NA₈-(C₁-C₆)alkyl-C(O)NA₇-(C₁-C₆)alkyl group, a NA₈-(C₁-C₆)alkyl-C(O)NA₇-(C₁-C₆)alkyl-(OCH₂CH₂)ᵢ group, a NA₈-(C₁-C₆)alkyl-NA₇-(C₁-C₆)alkyl group, a NA₈-(C₁-C₆)alkyl-NA₇-(C₁-C₆)alkyl-(OCH₂CH₂)ᵢ group, a NA₈-(C₁-C₆)alkyl-(OCH₂CH₂)ᵢ—NA₇-(C₁-C₆)alkyl group, a C(O)—NA₈-(C₁-C₆)alkyl-NA₇ group, a C(O)—NA₈-(C₁-C₆)alkyl-NA₇C(O)—(C₁-C₆)alkyl group, a C(O)—NA₈-(C₁-C₆)alkyl-NA₇C(O)—(C₁-C₆)alkyl-(OCH₂CH₂)ᵢ group, a C(O)—NA₈-(C₁-C₆)alkyl-C(O)NA₇-(C₁-C₆)alkyl group, a C(O)—NA₈-(C₁-C₆)alkyl-C(O)NA₇-(C₁-C₆)alkyl-(OCH₂CH₂)ᵢ group, a C(O)—NA₈-(C₁-C₆)alkyl-NA₇-(C₁-C₆)alkyl group, a C(O)—NA₈-(C₁-C₆)alkyl-NA₇-(C₁-C₆)alkyl-(OCH₂CH₂)ᵢ group, or a C(O)—NA₈-(C₁-C₆)alkyl-(OCH₂CH₂)ᵢ—NA₇-(C₁-C₆)alkyl group;
A₇ is a straight, branched, saturated, or unsaturated C₁-C₁₆₀ hydrocarbon; wherein one or more —CH₂— units of the C₁-C₁₆₀ hydrocarbon are optionally and independently replaced by one or more atoms or groups independently selected from the group consisting of —CH(OH)—, —CH(SO₃H)—, —CH(SO₃⁻M⁺)-, —C(O)—, —C(O)NH—, —C(O)N(alkyl)-, —C(O)O—, —NHC(O)—, —N(alkyl)C(O)—, —NHS(O)₂—, —N(alkyl)S(O)₂—, —P(O)(OH)—, —P(O)(OH)O—, —O—, —OC(O)—, —OC(O)O—, —OP(O)(OH)—, —OP(O)(OH)O—, —S(O)—, —S(O)₂—, —S(O)₂NH—, —S(O)₂N(alkyl)-, and heterocycloalkyl; wherein each heterocycloalkyl is optionally and independently substituted by one or more substituents independently selected from the group consisting of halogen, alkyl, NH₂, NH(alkyl), N(alkyl)₂, OH, and O(alkyl); and wherein the C₁-C₁₆₀ hydrocarbon is optionally substituted by one or more substituents independently selected from the group consisting of halogen, alkyl, NH₂, NH(alkyl), N(alkyl)₂, OH, and O(alkyl);
A₈ is hydrogen or (C₁-C₆)alkyl;
M⁺ is an alkali metal;
i is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41,42, 43, 44, 45, 46, 47, 48, 49, or 50; and
RCG1 is a reactive chemical group present at the end of L of formula (II), wherein RCG1 is reactive towards a chemical group present on a polypeptide.

24. The compound of claim 23, having the following structure:

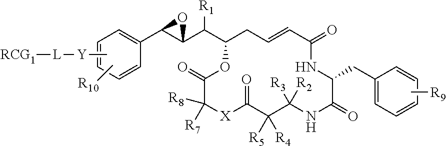

25. The compound of claim 23, wherein each (AAₙₛ) is independently selected from the group consisting of alanine (Ala), 7-aminobutyric acid (GABA), arginine (Arg), asparagine (Asn), aspartic acid (Asp), citrulline (Cit), cysteine (Cys), glutamine (Gln), glutamic acid (Glu), glycine (Gly), histidine (His), isoleucine (Ile), leucine (Leu), lysine (Lys), ε-(acetyl)-lysine ((ε-Ac)Lys), methionine (Met), phenylalanine (Phe), proline (Pro), serine (Ser), threonine (Thr), tryptophan (Trp), tyrosine (Tyr), and valine (Val).

26. The compound of claim 23, wherein each (AAₛ) is independently formula (VI): 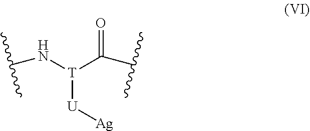
wherein:
T is a trivalent (C₁-C₈)alkyl;
U is -a single bond-, —C(O)—, —C(O)NH—, —C(O)N(alkyl)-, —C(O)O—, —NH—, —N(alkyl)-, —NHC(O)—, —N(alkyl)C(O)—, —NHC(NH)NH—, —NHC(O)NH—, —NHS(O)₂—, —N(alkyl)S(O)₂—, —O—, —OC(O)—, —OC(O)O—, —OP(O)—, —P(O)(OH)—, —P(O)(OH)O—, —OP(O)(OH)O—, —S—, —S(O)—, —S(O)₂—, —S(O)₂NH—, —S(O)₂N(alkyl)-, or —Se—;
A₉ is a straight, branched, saturated, or unsaturated C₁-C₁₆₀ hydrocarbon, wherein the C₁-C₁₆₀ hydrocarbon is optionally substituted by 1 or more substituents independently selected from the group consisting of halogen, alkyl, NH₂, NH(alkyl), N(alkyl)₂, OH, and O(alkyl), and further wherein 1 or more —CH₂—units of the C₁-C₁₆₀ hydrocarbon are optionally and independently replaced by 1 or more atoms or groups independently selected from the group consisting of —CHF—, —CF₂—, —CH(OH)—, —CH(Oalkyl)-, —CH(SO₃H)—, —CH(SO₃⁻M⁺)-, —C(O)—, —C(O)NH—, —C(O)N(alkyl)-, —C(O)O—, —NH—, —N⁺H(alkyl)-, —N(alkyl)-, —N⁺(alkyl)₂-, —NHC(O)—, —N(alkyl)C(O)—, —NHC(NH)NH—, —NHC(O)NH—, —NHS(O)₂—, —N(alkyl)S(O)₂—, —P(O)(OH)—, —P(O)(OH)O—, —O—, —OC(O)—, —OC(O)O—, —OP(O)—, —OP(O)(OH)—, —OP(O)(OH)O—, —S—, —S(O)—, —S(O)₂—, —S(O)₂NH—, —S(O)₂N(alkyl)-, —Se—, and heterocycloalkyl, wherein the heterocycloalkyl is optionally substituted by 1 or more substituents independently selected from the group consisting of halogen, alkyl, NH₂, NH(alkyl), N(alkyl)₂, OH, and O(alkyl); and
M⁺ is an alkali metal.

27. The compound of claim 26, wherein
T is 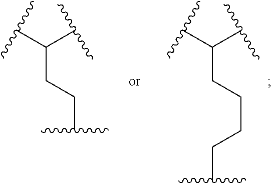 [SMILES: *CCC(*)* ; *CCCCC(*)*]
U is —C(O)NH— or —NHC(O)—;
A₉ is —[(CH₂)₂O]_{b}—CH₃; and
b is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

28. The compound of claim 23, wherein (AA)w represents a sequence of w non-substituted amino acids (AAₙₛ).

29. The compound of claim 28, wherein (AA)_{w} is Ala-Lys, Ala-Phe, Gly-Gly, Ile-Cit, Leu-Cit, Phe-Ala, Phe-Cit, Phe-Gly, Phe-Lys, Trp-Cit, Val-Ala, Val-Lys, Val-(ε-Ac)Lys, Val-Cit, Gly-Ala-Phe, Gly-Gly-Gly, Gly-Phe-Gly, Gly-Phe-Lys, Gly-Val-Cit, Phe-Phe-Lys, D-Phe-Phe-Lys, Ala-Leu-Ala-Leu, Gly-Phe-Leu-Cit, or Gly-Phe-Leu-Gly.

30. The compound of claim 28, wherein:
L2 is —(C₁-C₆)alkyl-NA₇-C(O)—(C₁-C₆)alkyl-;
A₇ is C(O)CH₂CH₂C(O)NHCH₂CH₂S(O)₂OH, C(O)CH₂CH₂C(O)NHCH₂CH₂S(O)₂OM⁺, C(O)CH₂CH₂C(O)NH—[CH₂CH₂O]ₐ—CH₂CH₂C(O)NHCH₂CH₂S(O)₂OH, C(O)CH₂CH₂C(O)NH—[CH₂CH₂O]ₐ—CH₂CH₂C(O)NHCH₂CH₂S(O)₂OM⁺, or C(O)—[CH₂CH₂O]ₐ—CH₃; and
a is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

31. The compound of claim 28, wherein A₇ is C(O)—[CH₂CH₂O]₄—CH₃, —C(O)—[CH₂CH₂O]₇—CH₃, C(O)—[CH₂CH₂O]₂₄—CH₃, C(O)CH₂CH₂C(O)NHCH₂CH₂S(O)₂OH, C(O)CH₂CH₂C(O)NHCH₂CH₂S(O)₂OM⁺, C(O)CH₂CH₂C(O)NH—[CH₂CH₂O]₄—CH₂CH₂C(O)NHCH₂CH₂S(O)₂OH, or C(O)CH₂CH₂C(O)NH—[CH₂CH₂O]₄—CH₂CH₂C(O)NHCH₂CH₂S(O)₂OM⁺.

32. The compound of claim 28, wherein L2 is selected from the group consisting of:

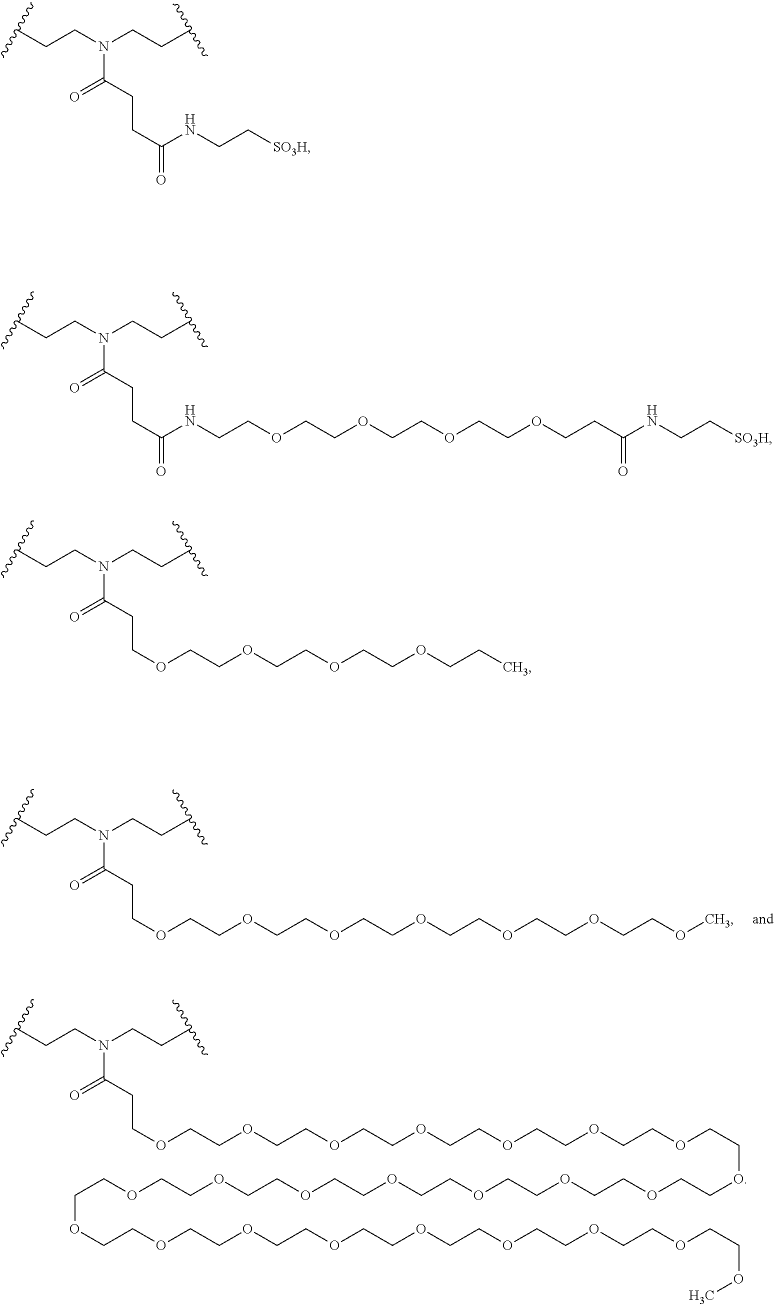 [SMILES: *CCN(CC*)C(=O)CCC(=O)NCCS(=O)(=O)O ; *CCN(CC*)C(=O)CCC(=O)NCCOCCOCCOCCOCCC(=O)NCCS(=O)(=O)O ; *CCN(CC*)C(=O)CCOCCOCCOCCOCCC ; *CCN(CC*)C(=O)CCOCCOCCOCCOCCOCCOCCOC ; *CCN(CC*)C(=O)CCOCCOCCOCCOCCOCCOCCOCCOCCOCCOCCOCCOCCOCCOCCOCCOCCOCCOCCOCCOCCOCCOCCOCCOC]

33. The compound of claim 28, wherein L is selected from the group consisting of:

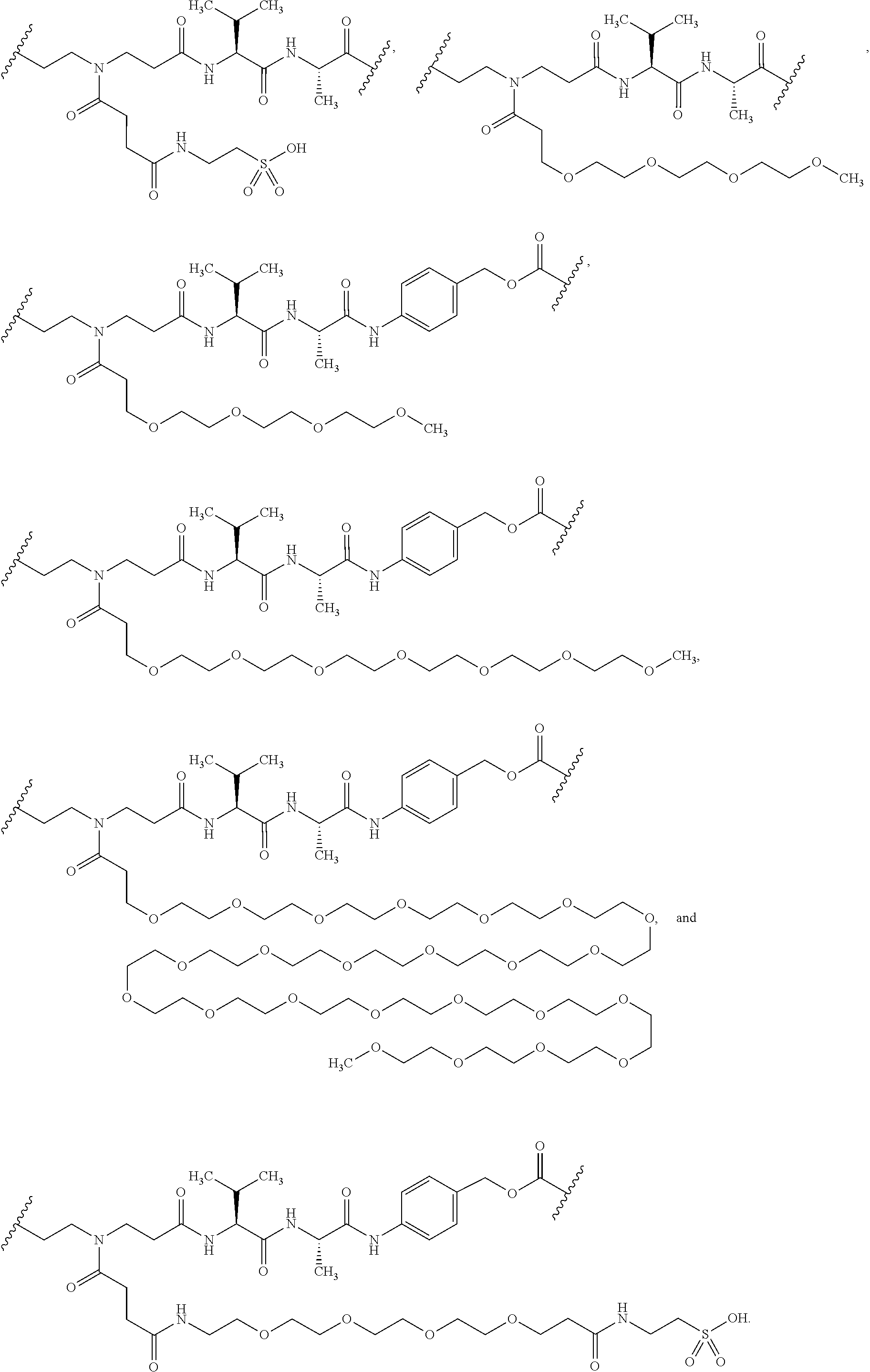

34. The compound of claim 23, wherein (AA)w contains at least one substituted amino acid (AAₛ).

35. The compound of claim 34, wherein:
L2 is —(C₁-C₆)alkyl-, —C(O)—(C₁-C₆)alkyl-, or —C(O)—(C₁-C₆)alkyl-NA₇-(C₁-C₆)alkyl-;
A₇ is —C(O)CH₂CH₂C(O)NHCH₂CH₂SO₃H or —C(O)CH₂CH₂C(O)NHCH₂CH₂SO₃-M⁺; and
M⁺ is an alkali metal.

36. The compound of claim 34, wherein (AA)w is selected from the group consisting of:

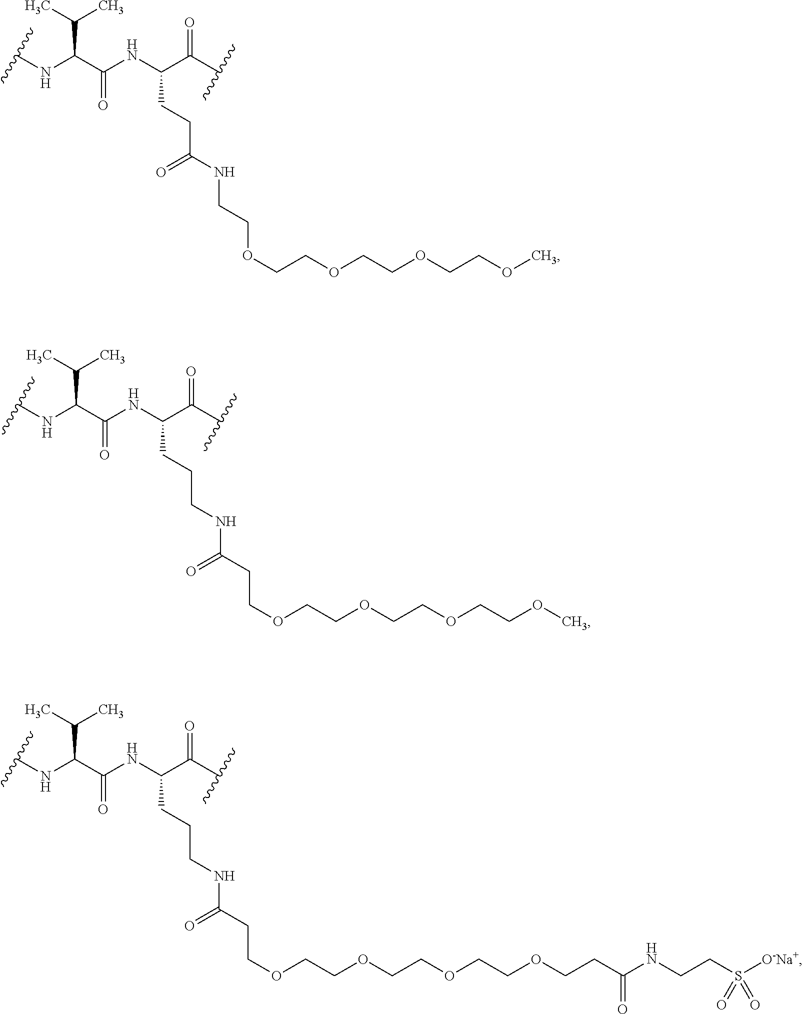

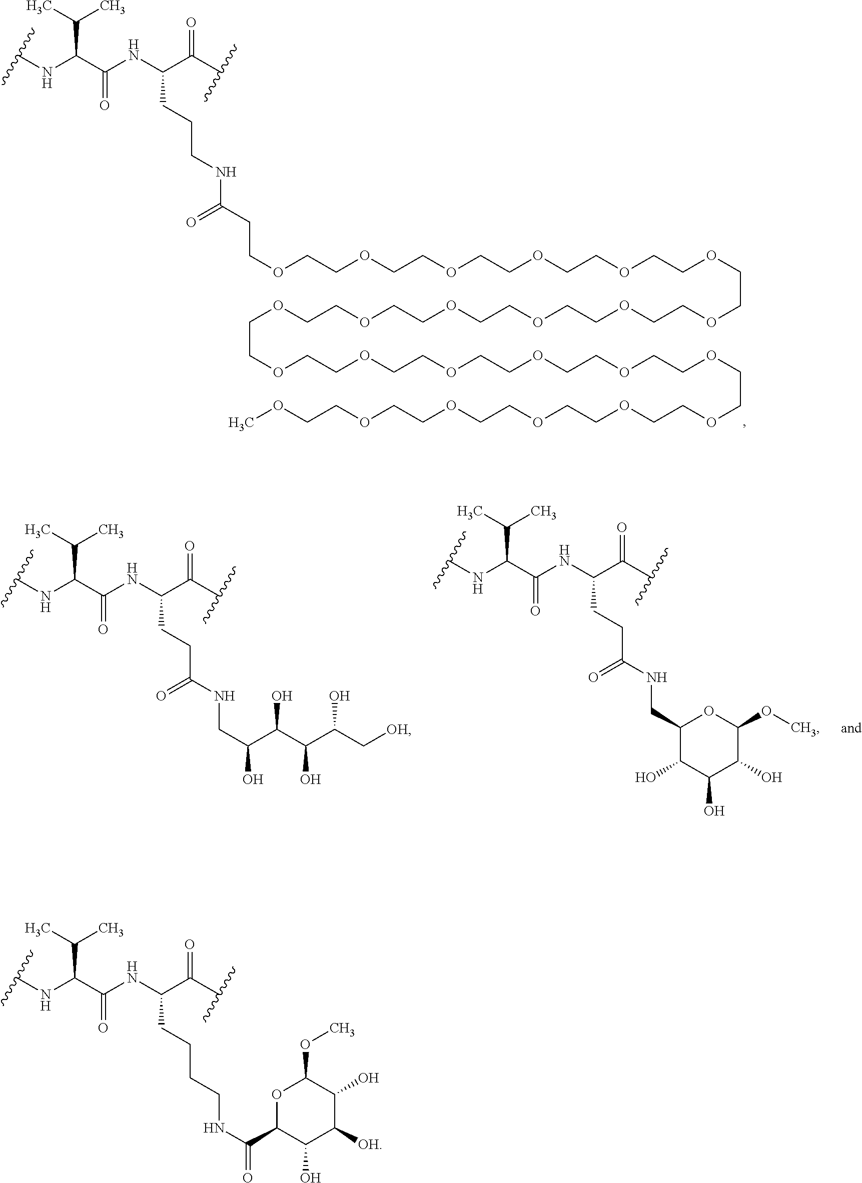 [SMILES: *N[C@H](C(=O)N[C@@H](CCCNC(=O)CCOCCOCCOCCOCCOCCOCCOCCOCCOCCOCCOCCOCCOCCOCCOCCOCCOCCOCCOCCOCCOCCOCCOCCOC)C(*)=O)C(C)C ; *N[C@H](C(=O)N[C@@H](CCC(=O)NC[C@H](O)[C@@H](O)[C@H](O)[C@H](O)CO)C(*)=O)C(C)C ; *N[C@H](C(=O)N[C@@H](CCC(=O)NC[C@H]1O[C@@H](OC)[C@H](O)[C@@H](O)[C@@H]1O)C(*)=O)C(C)C ; "and" ; *N[C@H](C(=O)N[C@@H](CCCCNC(=O)[C@H]1O[C@@H](OC)[C@H](O)[C@@H](O)[C@@H]1O)C(*)=O)C(C)C]

37. The compound of claim 34, wherein L is selected from the group consisting of:

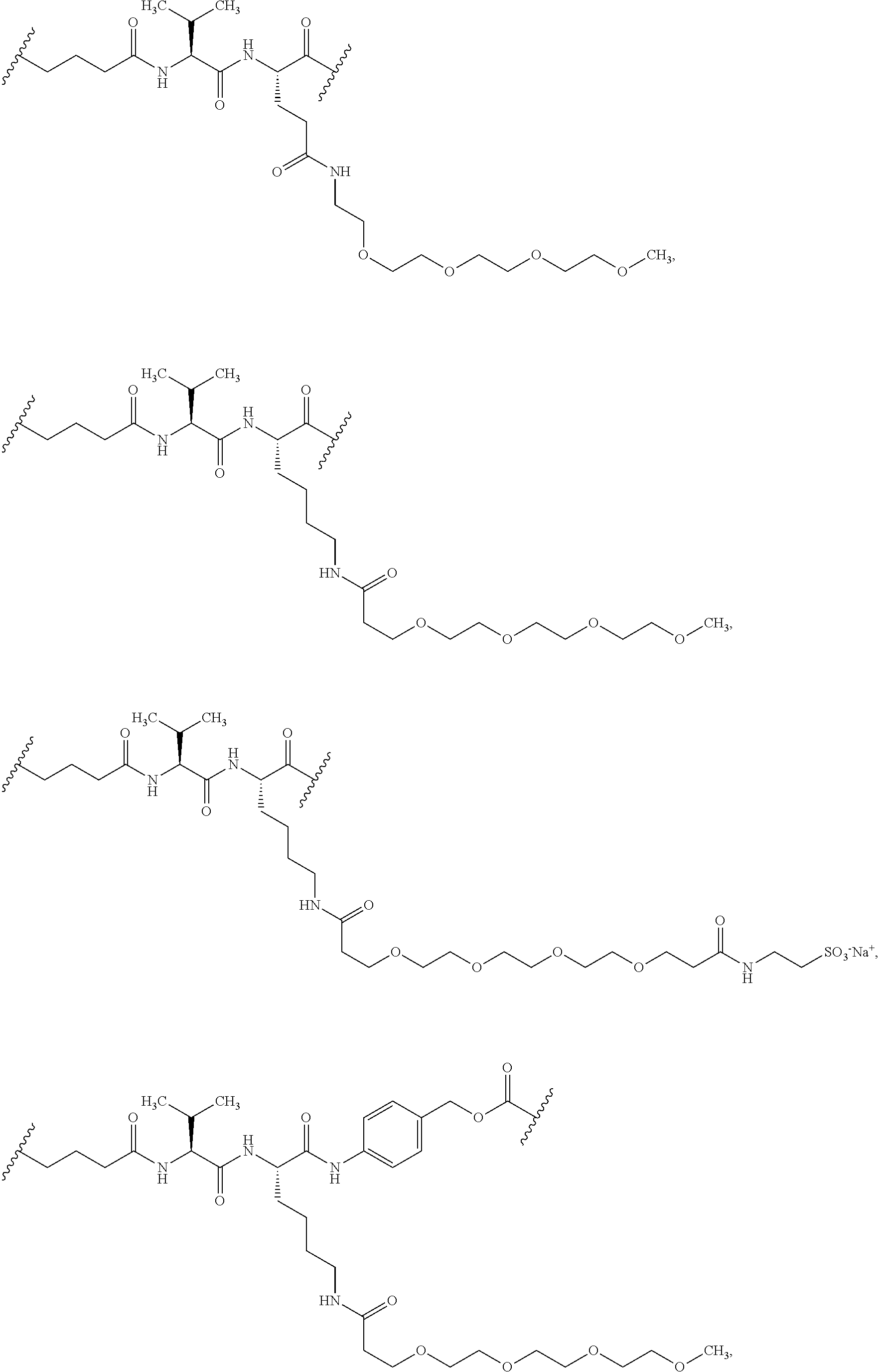

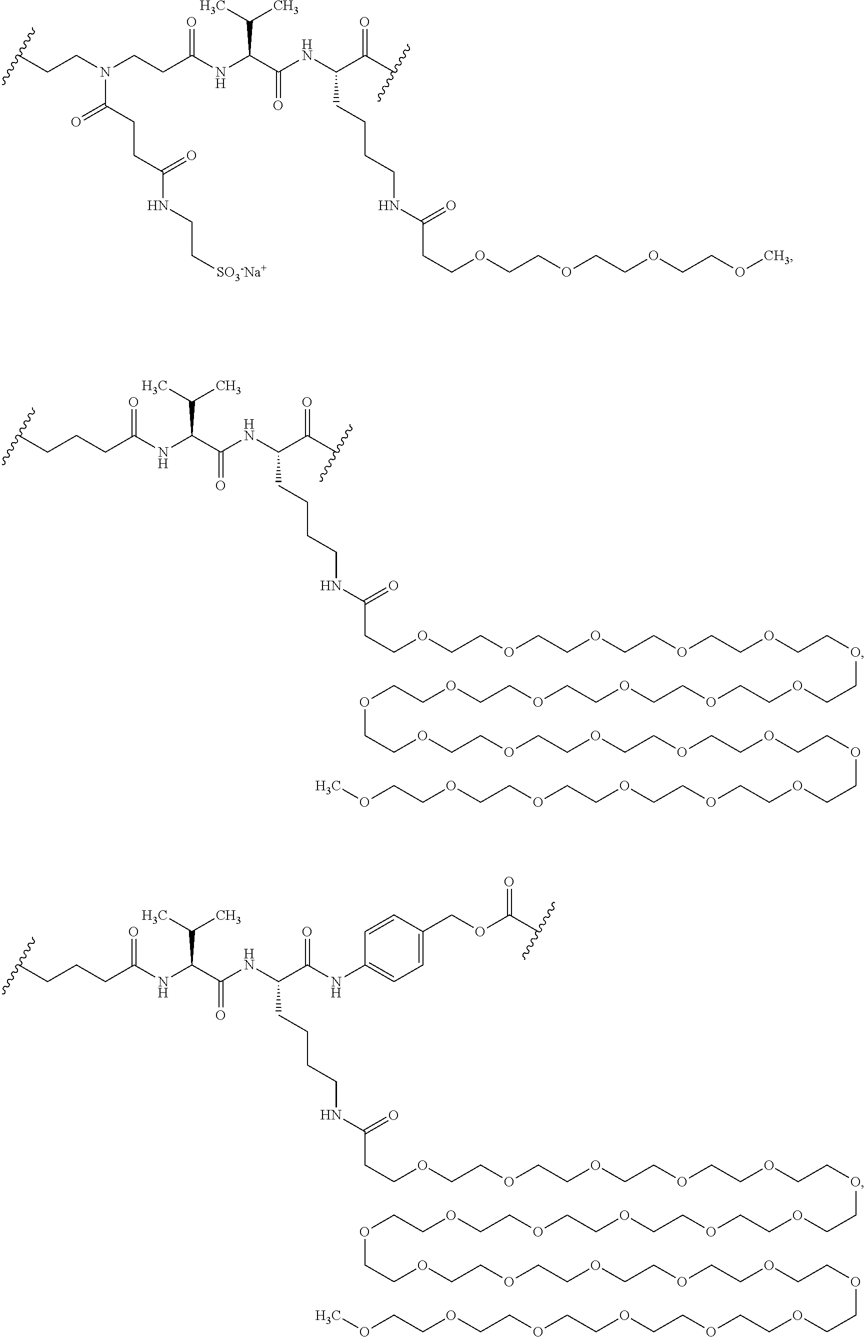

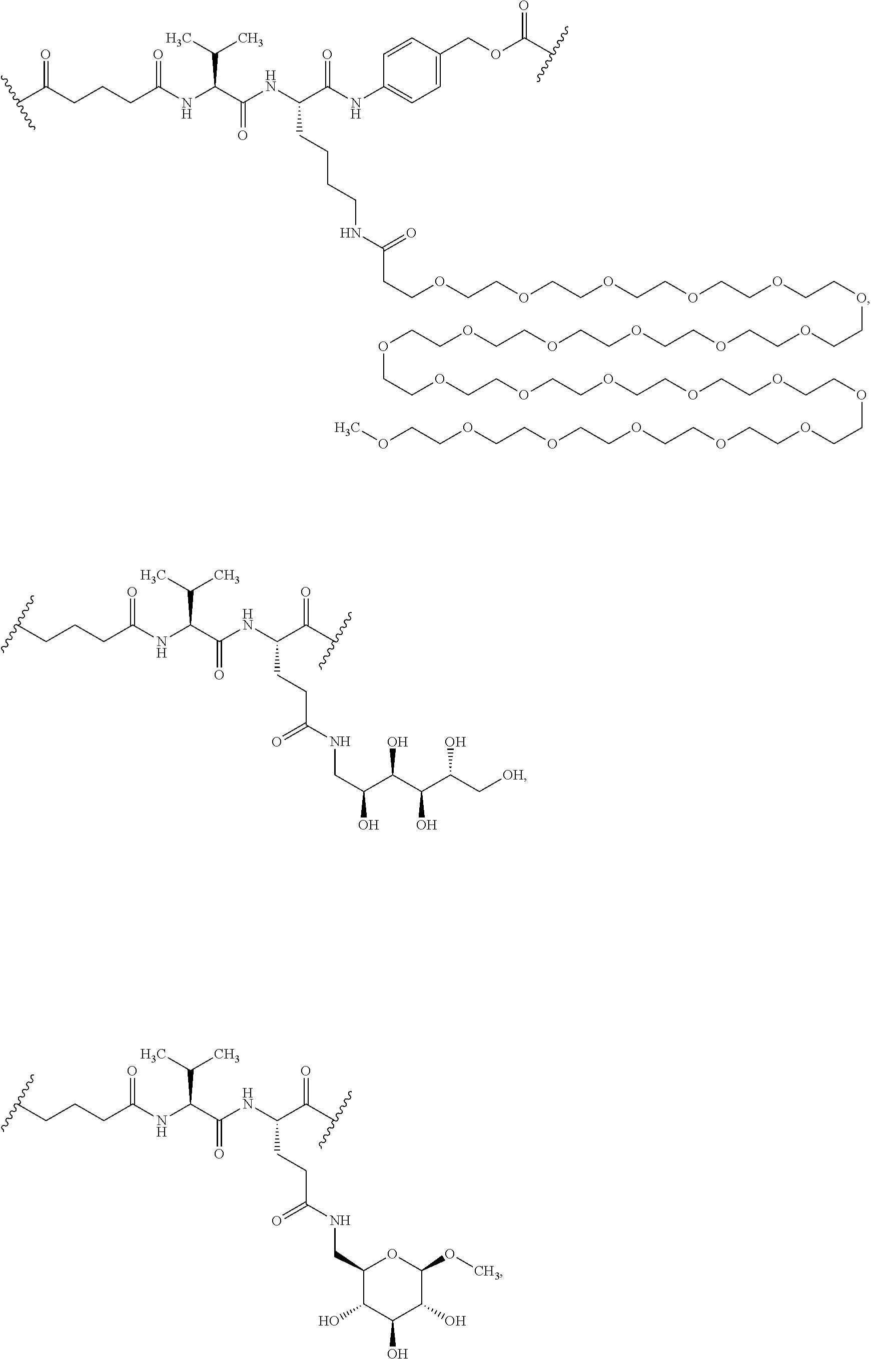 [SMILES: *C(=O)CCCC(=O)N[C@H](C(=O)N[C@@H](CCCCNC(=O)CCOCCOCCOCCOCCOCCOCCOCCOCCOCCOCCOCCOCCOCCOCCOCCOCCOCCOCCOCCOCCOCCOCCOCCOC)C(=O)Nc1ccc(COC(*)=O)cc1)C(C)C ; *CCCC(=O)N[C@H](C(=O)N[C@@H](CCC(=O)NC[C@H](O)[C@@H](O)[C@H](O)[C@H](O)CO)C(*)=O)C(C)C ; *CCCC(=O)N[C@H](C(=O)N[C@@H](CCC(=O)NC[C@H]1O[C@@H](OC)[C@H](O)[C@@H](O)[C@@H]1O)C(*)=O)C(C)C]

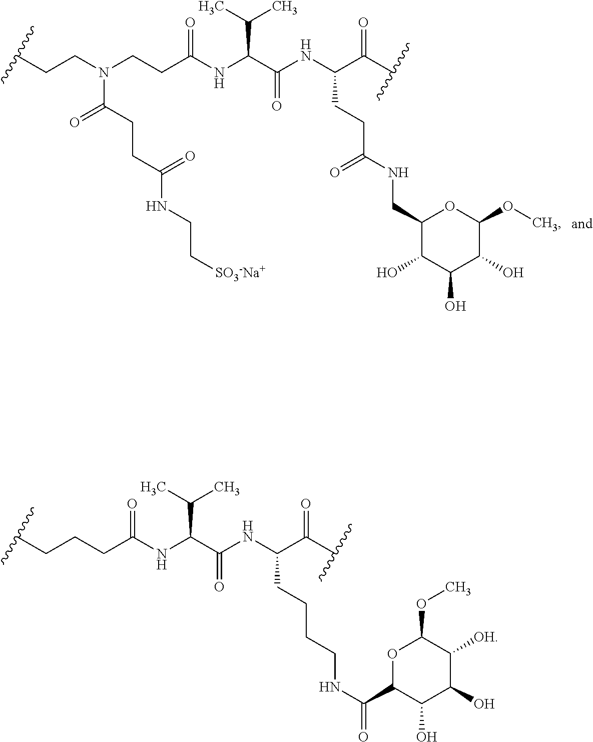

38. The compound of claim 23, wherein RCG1 is reactive towards a chemical group present on an antibody.

39. The compound of claim 23, wherein:
RCG1 is:
C₁, C≡CH, NH₂, N₃, OH, O(alkyl)-NHOH, SH, 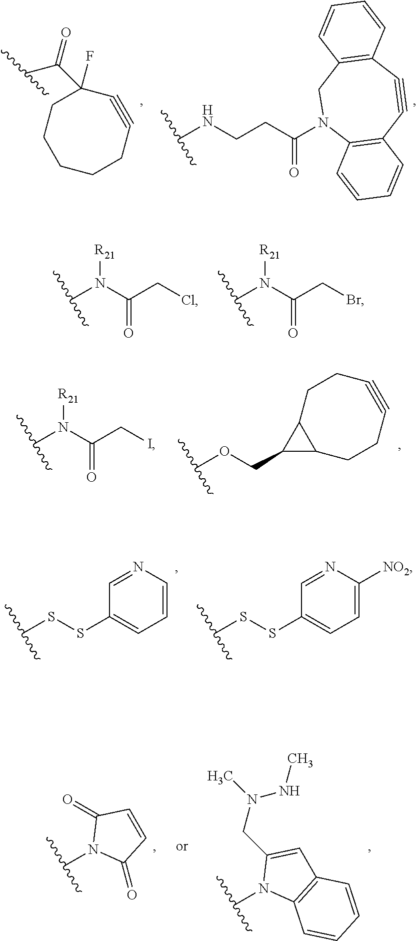
wherein:
R₂₁ is hydrogen or (C₁-C₆) alkyl; and
the —CO—, —NH—, —NR₂₁—, —N═, —O—, or S is bonded to L2; or
RCG1 is:
C(O)ZₐRₐ,
wherein:
Zₐ is a single bond, —NH—, or —O—; and
Rₐ is hydrogen, (C₁-C₆)alkyl, alkenyl, (C₃-C₇)cycloalkyl, (C₃-C₇)heterocycloalkyl, aryl, or heteroaryl, wherein the (C₃-C₇)heterocycloalkyl, aryl, or heteroaryl is optionally substituted by 1, 2, 3, 4, or 5 substituents independently selected from the group consisting of halogen, CN, NO₂, alkyl, OH, O(alkyl), and =0;
wherein —C(O)— is bonded to L2.

40. The compound of claim 23, selected from the group consisting of: 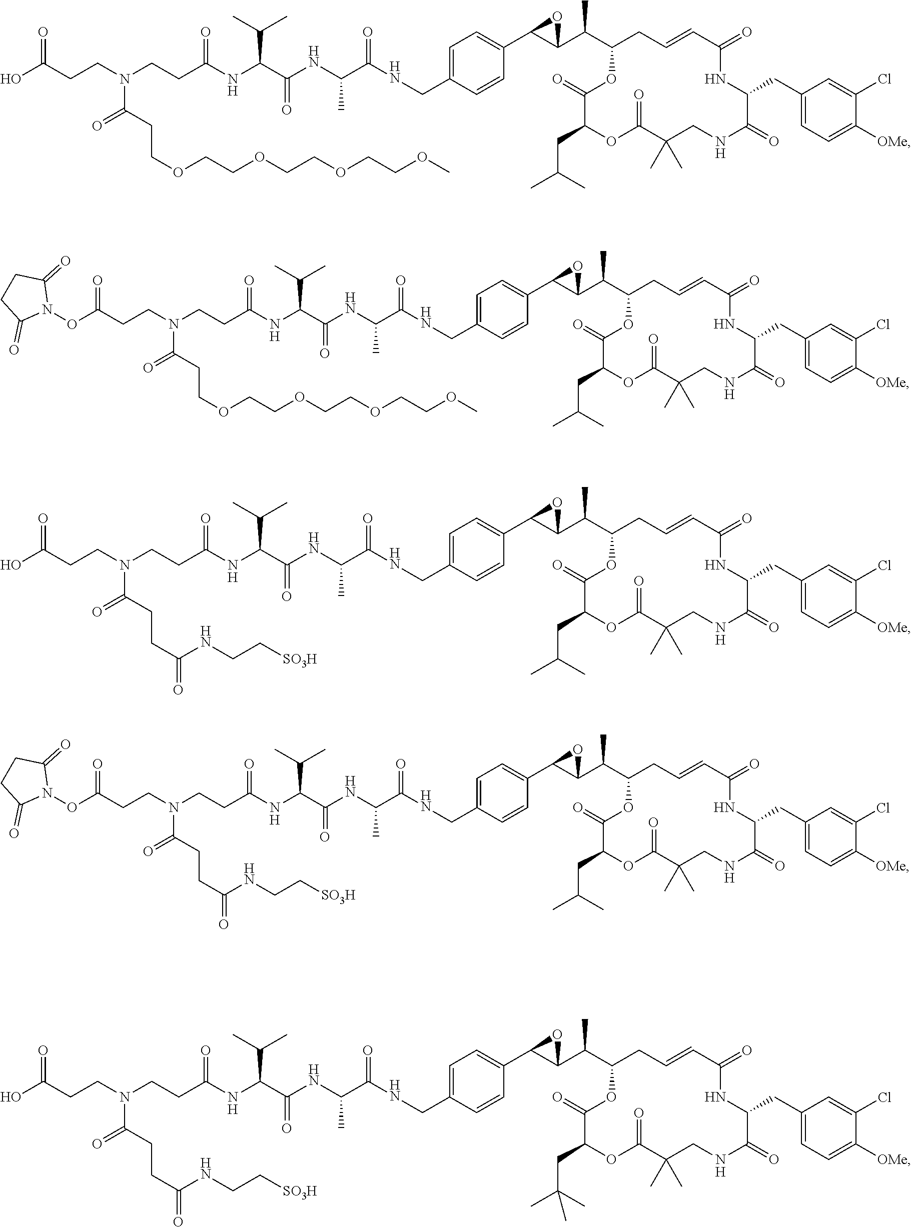 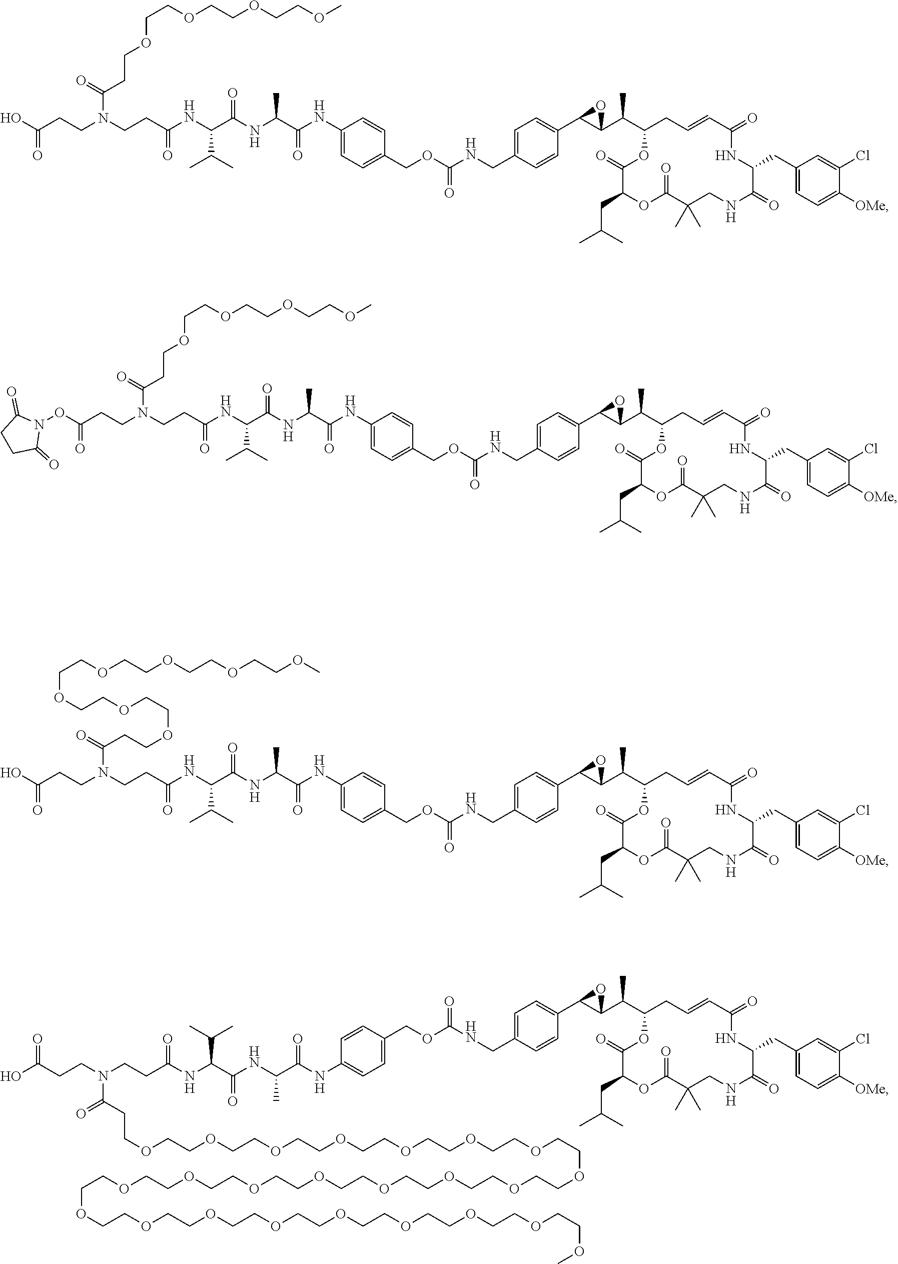 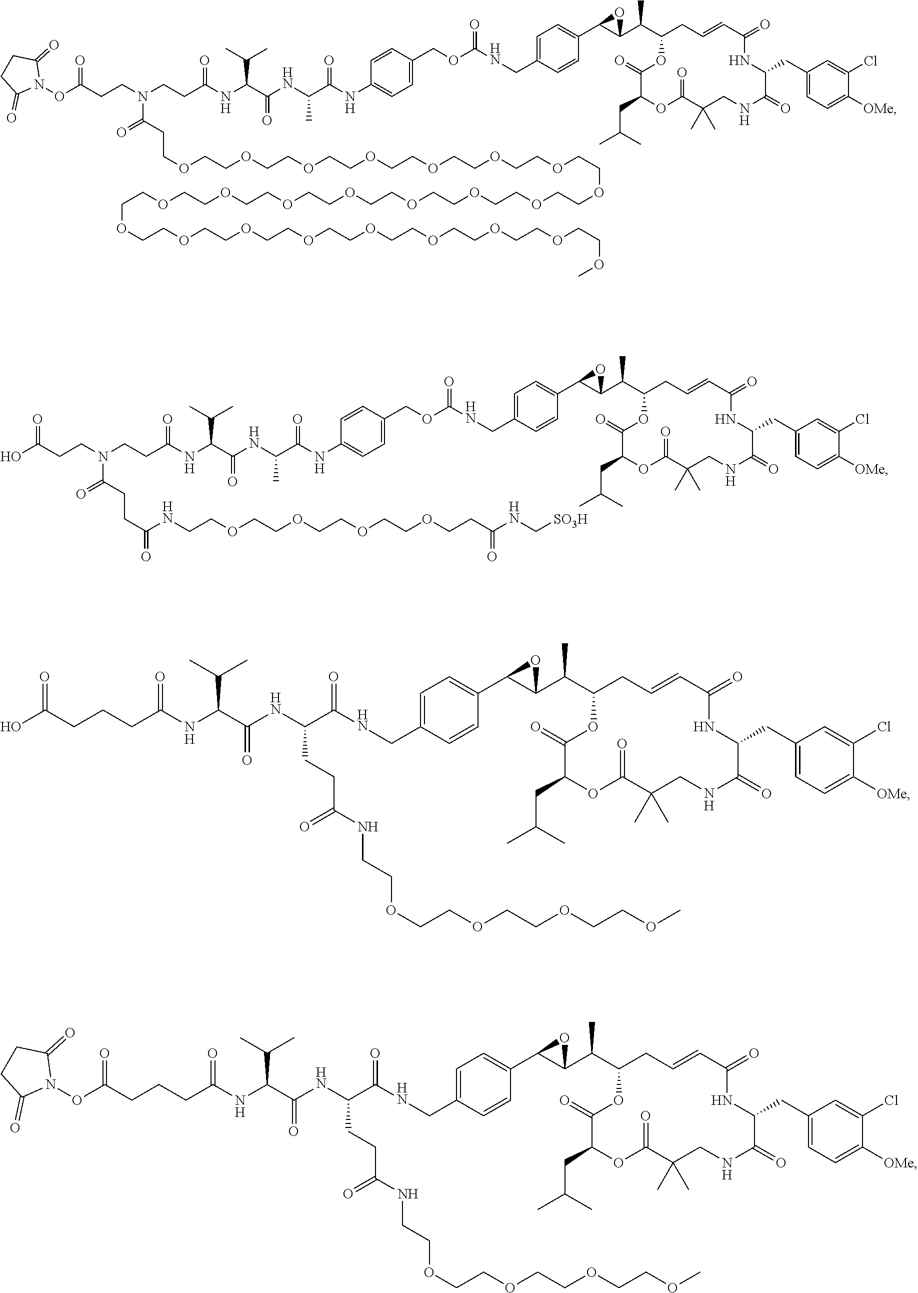 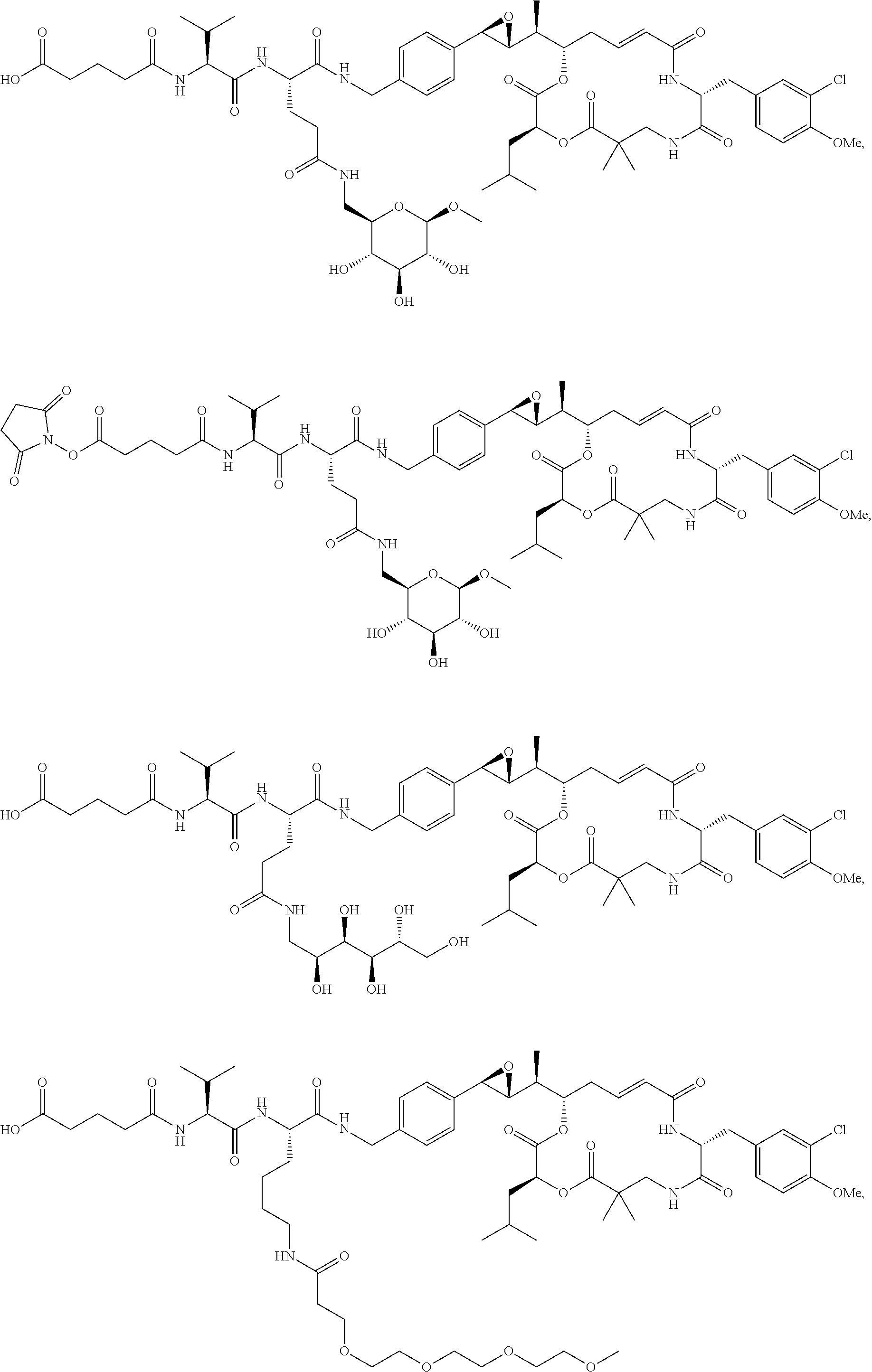 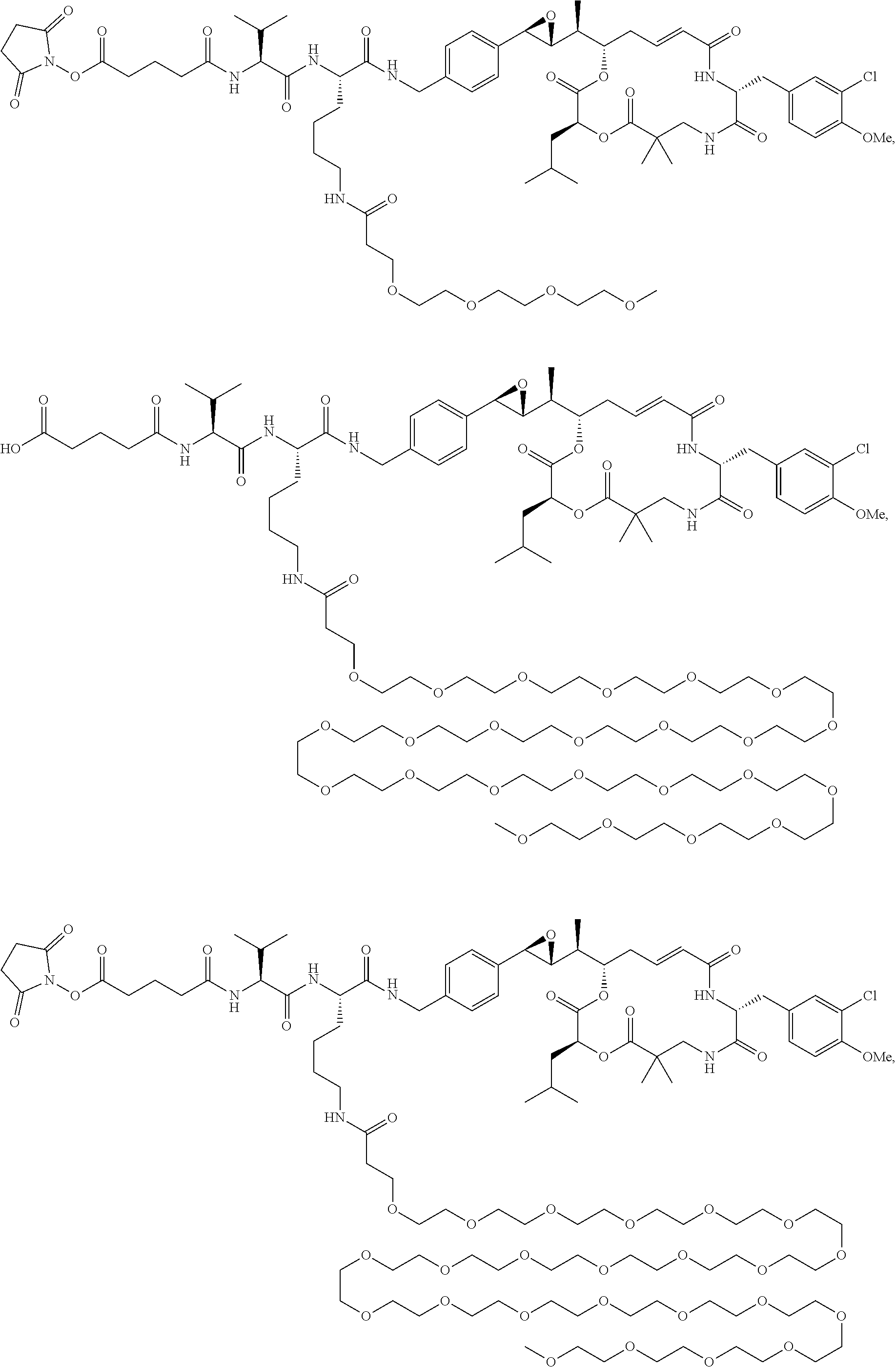 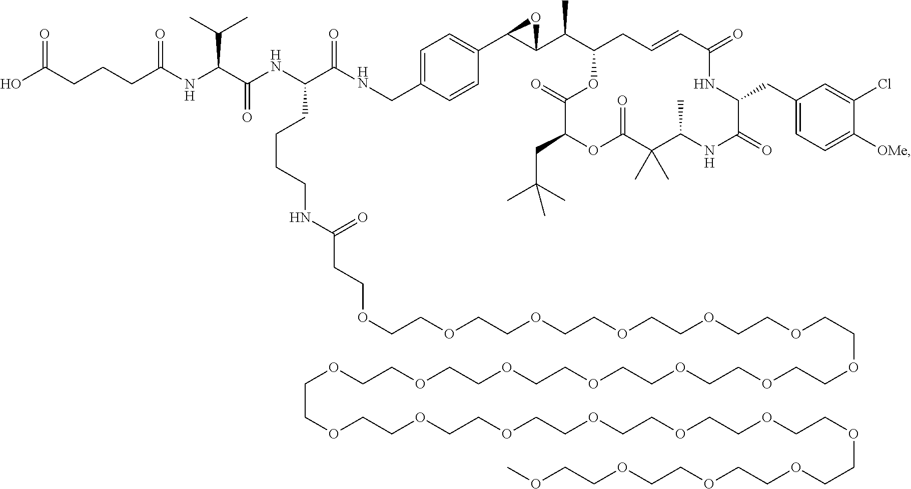 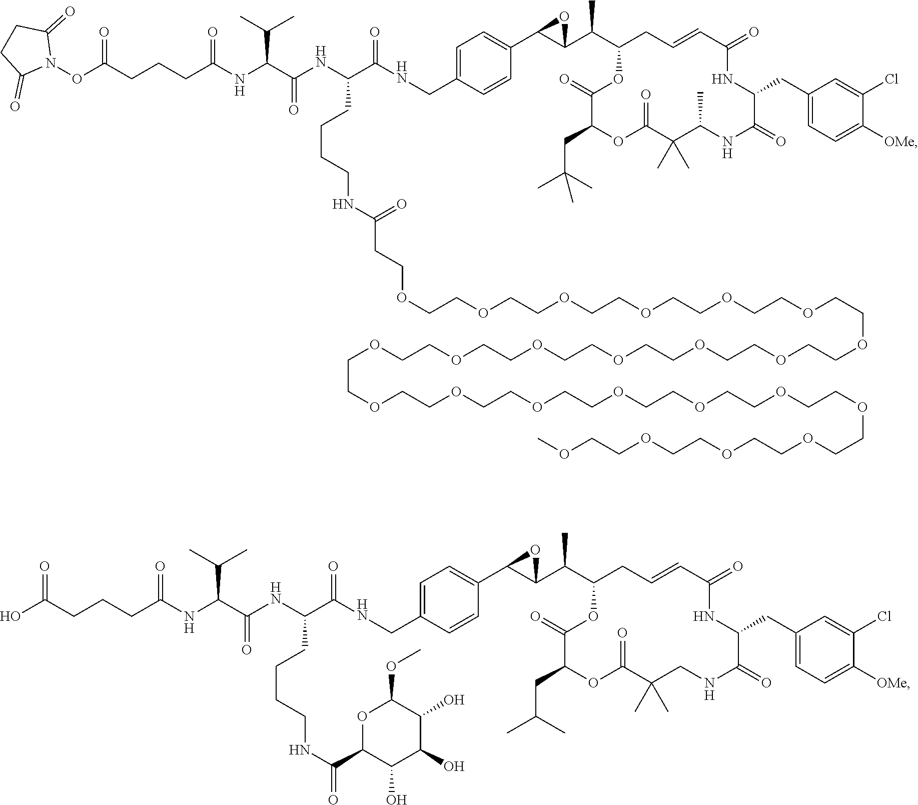 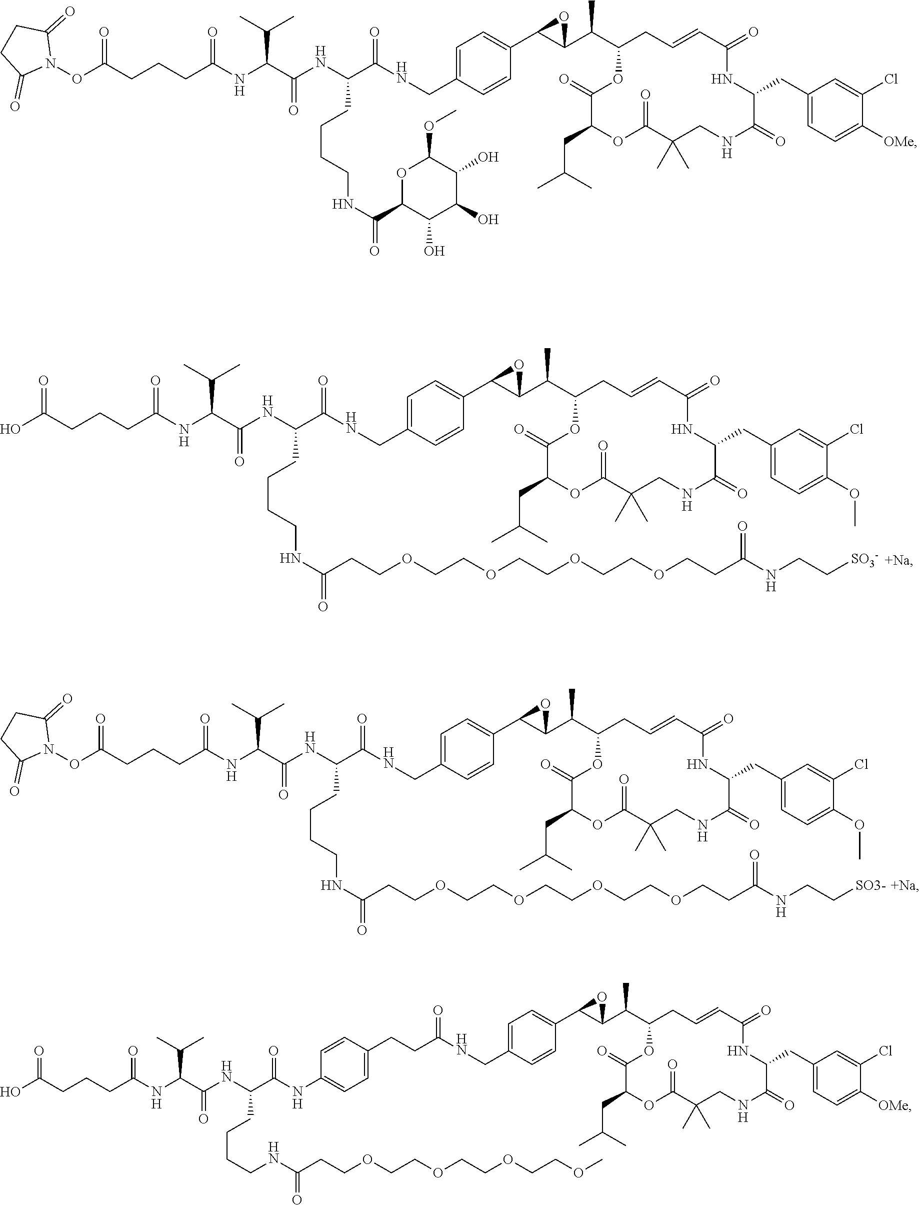 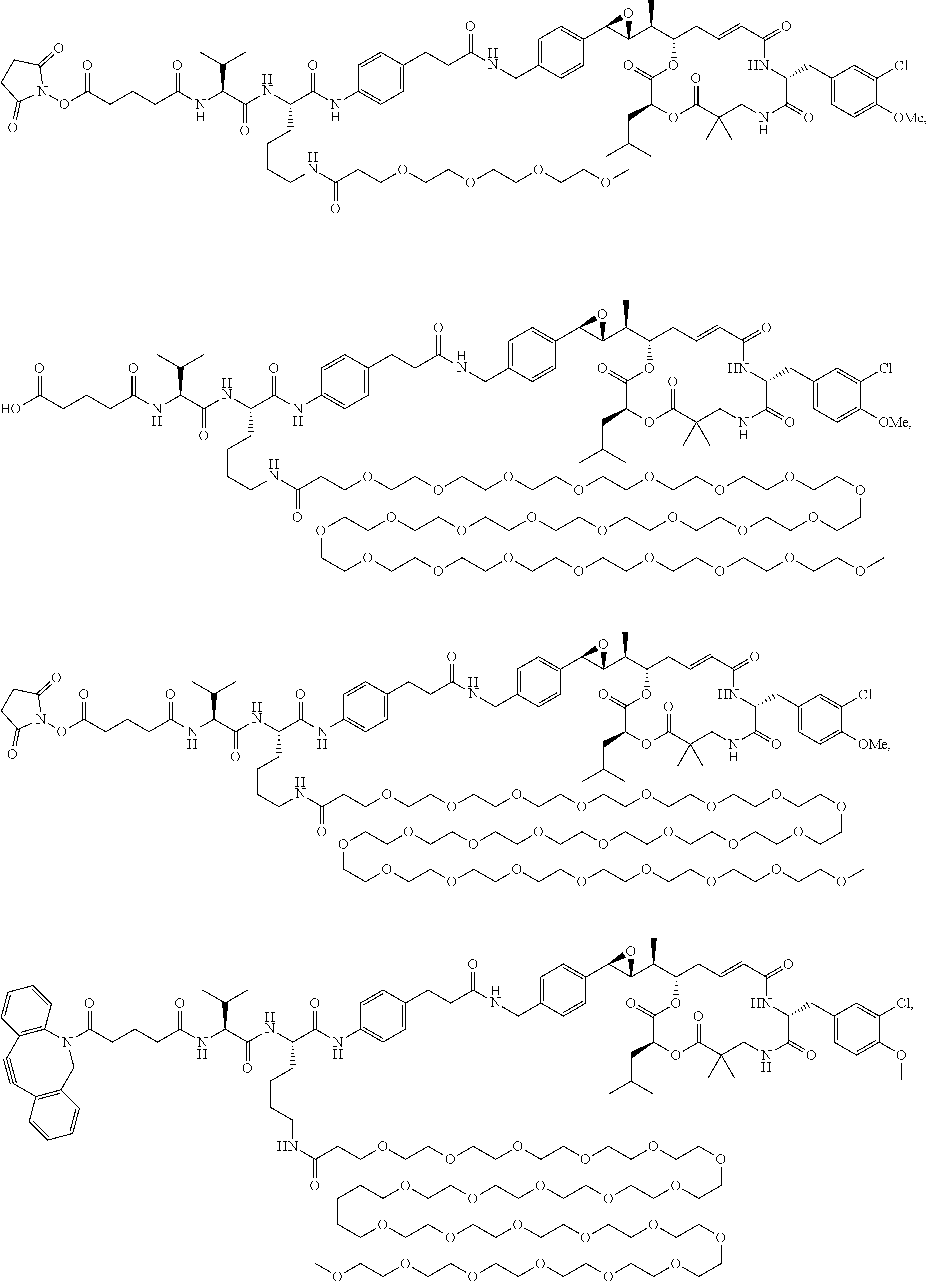 [chemical structure]
